(19) United States
(12) Patent Application Publication
(10) Pub. No.: US 2024/0238412 A1
(43) Pub. Date: Jul. 18, 2024

(54) PANCORONAVIRUS VACCINES

(71) Applicant: Gritstone bio, Inc., Emeryville, CA (US)

(72) Inventors: Leonid Gitlin, Foster City, CA (US); Karin Jooss, San Diego, CA (US); Sue-Jean Hong, Emeryville, CA (US); Ciaran Daniel Scallan, San Francisco, CA (US); Amy Rachel Rappaport, Daly City, CA (US); Christine Denise Palmer, Cambridge, MA (US); Minh Duc Cao, Belmont, MA (US); Joshua Klein, Brookline, MA (US)

(21) Appl. No.: 18/622,497

(22) Filed: Mar. 29, 2024

Related U.S. Application Data
(63) Continuation of application No. PCT/US2022/077488, filed on Oct. 3, 2022.
(60) Provisional application No. 63/374,664, filed on Sep. 6, 2022.
(60) Provisional application No. 63/251,441, filed on Oct. 1, 2021.

(51) Int. Cl. A61K 39/215; A61K 39/00; A61P 37/04; C12N 15/86

(52) U.S. Cl. CPC A61K 39/215; A61P 37/04; C12N 15/86; A61K 2039/5256; C12N 2770/20022; C12N 2770/20034; C12N 2770/36143; C12N 2830/50

(57) ABSTRACT

Disclosed herein are vaccine compositions that include Pancorona receptor binding domain (RBD) encoding cassettes and/or MHC epitope-encoding cassettes. Also disclosed are nucleotides, cells, and methods associated with the compositions including their use as vaccines.

### CROSS REFERENCE TO RELATED APPLICATIONS

This application is a Continuation of International Patent Application No. PCT/US2022/077488, filed Oct. 3, 2022, which claims the benefit of U.S. Provisional Application No. 63/251,441 filed Oct. 1, 2021 and U.S. Provisional Application No. 63/374,664 filed Sep. 6, 2022, each of which is hereby incorporated in their entirety by reference for all purposes.

### SEQUENCE LISTING

The instant application contains a Sequence Listing which has been submitted electronically in .XML format and is hereby incorporated by reference in its entirety. Said XML copy, created on 17 Feb. 2023, is named GSO-102WO and is 5,884,562 bytes in size.

### BACKGROUND

Severe acute respiratory syndrome corona virus 2 (SARS-COV-2) is the virus strain responsible for the Coronavirus Disease 2019 (Covid-19) pandemic. As of Dec. 21, 2021, the virus has infected over 275 million people and caused about 5.4 million deaths worldwide. A CD8+ T cell response may be important for COVID-19 for two reasons in a coronavirus context. First is the recurrent observation in pre-clinical models that SARS vaccines that only stimulate antibody responses are often associated with pulmonary inflammation, independent of viral clearance. This has been observed in both rodents and non-human primates (NHP), and the current consensus is that it is caused by an imbalanced immune response, and is likely to be solved by using vaccines that drive a balanced antibody and CD8+ T cell (Th1) response (Consensus considerations on the assessment of the risk of disease enhancement with COVID-19 vaccines: Outcome of a Coalition for Epidemic Preparedness Innovations (CEPI)/Brighton Collaboration (BC) scientific working meeting, Mar. 12-13, 2020). Secondly, coronaviruses are evidently mutating frequently and crossing from animal reservoirs into humans, with three epidemics/pandemics over the last 18 years (SARS in 2002, MERS in 2012, now COVID-19). Antibody responses are often against highly mutable proteins (such as the Spike protein of SARS-COV-2) which change significantly between strains and isolates, whereas T cell epitopes often derive from more evolutionarily conserved proteins. T cell memory is also generally more durable than B cell memory and thus CD8+T memory against SARS-COV-2 may provide longer, and better protection against future SARS variants. Many vaccines have demonstrated an ability to drive antibody responses in NHP and humans, but commonly used modalities such as protein/peptide and mRNA vaccines have not stimulated meaningful CD8+ T cell responses in these species.

An additional question for antigen vaccine design in infectious disease settings is which of the many proteins present generate the “best” therapeutic antigens, e.g., antigens that can stimulate immunity.

In addition to the challenges of current antigen prediction methods certain challenges also exist with the available vector systems that can be used for antigen delivery in humans, many of which are derived from humans. For example, many humans have pre-existing immunity to human viruses as a result of previous natural exposure, and this immunity can be a major obstacle to the use of recombinant human viruses for antigen delivery in vaccination strategies, such as in cancer treatment or vaccinations against infectious diseases.

While some progress has been made in vaccinations strategies addressing the above problems, improvements are still needed, particularly for clinical applications, such as improved vaccine potency and efficacy, such as the need for a pan-Coronavirus vaccine that stimulates balanced B and T cell immunity in humans against current and potential future Coronavirus.

### SUMMARY

Provided for herein is a composition for delivery of an antigen expression system, comprising: the antigen expression system, wherein the antigen expression system comprises at least two distinct coronavirus receptor binding domain (RBD) derived nucleic acid sequences encoding at least two distinct RBD domains, respectively, and wherein the at least two distinct RBD domains are collectively at least 70% identical by amino acid composition to RBD domains from at least two of a clade 1 Sarbecovirus, a clade 2 Sarbecovirus, or a clade 3 Sarbecovirus.

Also provided herein is a composition for delivery of an antigen expression system, comprising: the antigen expression system, wherein the antigen expression system comprises: (a) a vector comprising: a vector backbone, wherein the vector backbone comprises: (i) at least one promoter nucleotide sequence, and (ii) at least one polyadenylation (poly(A)) sequence; and (b) an antigen cassette, wherein the antigen cassette is inserted into the vector backbone such that the antigen cassette is operably linked to the at least one promoter nucleotide sequence, and wherein the antigen cassette comprises at least two distinct coronavirus receptor binding domain (RBD) derived nucleic acid sequences encoding at least two distinct RBD domains, respectively, and wherein the at least two distinct RBD domains are collectively at least 70% identical by amino acid composition to RBD domains from at least two of a clade 1 Sarbecovirus, a clade 2 Sarbecovirus, or a clade 3 Sarbecovirus.

In some aspects, the at least two distinct RBD domains are collectively at least 70% identical by amino acid composition to RBD domains from each of: (A) the clade 3 Sarbecovirus; and (B) the clade 1 Sarbecovirus and/or the clade 2 Sarbecovirus. In some aspects, the at least two distinct RBD domains are collectively at least 80% identical by amino acid composition to RBD domains from each of the clade 1 Sarbecovirus and the clade 2 Sarbecovirus. In some aspects, the at least two distinct RBD domains are collectively at least 85% identical by amino acid composition to RBD domains from each of the clade 1 Sarbecovirus and the clade 2 Sarbecovirus. In some aspects, the at least two distinct RBD domains are collectively at least 90% identical by amino acid composition to RBD domains from each of the clade 1 Sarbecovirus and the clade 2 Sarbecovirus.

In some aspects, the at least two distinct RBD domains are collectively at least 70% identical by amino acid composition to RBD domains from each of NC_045512.2, NC_004718.3, MT121216.1, DQ648857.1, GQ153542.1, DQ648856.1, AY278489.2, GQ153540.1, MN996532.2, KJ473811.1, NC_014470.1, KC881005.1, MK211377.1, KJ473816.1, MK211376.1, AY572034.1, KP886809.1, MT072864.1, KF569996.1, JX993987.1, MK211378.1, MK211374.1, KJ473815.1, JX993988.1, DQ071615.1, KT444582.1, MZ206298.1, and KJ473814.1.

In some aspects, the antigen expression system comprises at least three distinct coronavirus RBD derived nucleic acid sequences encoding at least three distinct RBD domain. In some aspects, the at least three distinct RBD domains are collectively at least 70% identical by amino acid composition to RBD domains from each of the clade 1 Sarbecovirus, the clade 2 Sarbecovirus, and the clade 3 Sarbecovirus. In some aspects, the at least three distinct RBD domains are collectively at least 70% identical by amino acid composition to RBD domains from each of NC_045512.2, NC_004718.3, MT121216.1, DQ648857.1, GQ153542.1, DQ648856.1, AY278489.2, GQ153540.1, MN996532.2, KJ473811.1, NC_014470.1, KC881005.1, MK211377.1, KJ473816.1, MK211376.1, AY572034.1, KP886809.1, MT072864.1, KF569996.1, JX993987.1, MK211378.1, MK211374.1, KJ473815.1, JX993988.1, DQ071615.1, KT444582.1, MZ206298.1, KJ473814.1, and SARS-COV-2.

In some aspects, the antigen expression system comprises at least four distinct coronavirus RBD derived nucleic acid sequences encoding at least four distinct RBD domain. In some aspects, the at least four distinct RBD domains are collectively at least 70% identical by amino acid composition to RBD domains from each of the clade 1 Sarbecovirus, the clade 2 Sarbecovirus, and the clade 3 Sarbecovirus. In some aspects, the at least four distinct RBD domains are collectively at least 70% identical by amino acid composition to RBD domains from each of NC_045512.2, NC_004718.3, MT121216.1, DQ648857.1, GQ153542.1, DQ648856.1, AY278489.2, GQ153540.1, MN996532.2, KJ473811.1, NC_014470.1, KC881005.1, MK211377.1, KJ473816.1, MK211376.1, AY572034.1, KP886809.1, MT072864.1, KF569996.1, JX993987.1, MK211378.1, MK211374.1, KJ473815.1, JX993988.1, DQ071615.1, KT444582.1, MZ206298.1, KJ473814.1, and SARS-COV2.

In some aspects, each of the sarbecovirus RBD derived nucleic acid sequences are independently derived from RBD nucleic acid sequences of sarbecovirus sequences selected from the group consisting of: KP886809, KJ473815, MK211376, DQ648856, GQ153542, NC_004718, JX993988, and SARS-COV2. In some aspects, each of the sarbecovirus RBD derived nucleic acid sequences are independently derived from RBD nucleic acid sequences of sarbecovirus sequences from each of KP886809, KJ473815, MK211376, and SARS-COV2. In some aspects, each of the sarbecovirus RBD derived nucleic acid sequences are independently derived from RBD nucleic acid sequences of sarbecovirus sequences from each of KJ473815, MK211376, DQ648856, and SARS-COV2. In some aspects, each of the sarbecovirus RBD derived nucleic acid sequences are independently derived from RBD nucleic acid sequences of sarbecovirus sequences from each of GQ153542, NC_004718, JX993988, and SARS-COV2.

In some aspects, the at least two distinct RBD domains encode full-length RBD domains that are collectively at least 70% identical by amino acid composition to full-length RBD domains from at least two of the clade 1 Sarbecovirus, the clade 2 Sarbecovirus, or the clade 3 Sarbecovirus. In some aspects, the at least two distinct RBD domains encode full-length RBD domains that are collectively at least 70% identical by amino acid composition to full-length RBD domains from at least two of the clade 1 Sarbecovirus, the clade 2 Sarbecovirus, or the clade 3 Sarbecovirus. In some aspects, the at least two distinct coronavirus RBD derived nucleic acid sequences comprise at least a betacoronavirus RBD derived nucleic acid sequence.

In some aspects, the at least two distinct coronavirus RBD derived nucleic acid sequences each comprise a betacoronavirus RBD derived nucleic acid sequences. In some aspects, the at least two distinct coronavirus RBD derived nucleic acid sequences are selected from the group consisting of: a betacoronavirus RBD derived nucleic acid sequence, an alphacoronavirus RBD derived nucleic acid sequence, and combinations thereof.

In some aspects, each of the distinct RBD domains comprises a distinct receptor-binding motif (RBM) domain, respectively, and wherein the distinct RBM domains are collectively at least 30% identical by amino acid composition to RBM domains from at least two of a clade 1 Sarbecovirus, a clade 2 Sarbecovirus, or a clade 3 Sarbecovirus.

In some aspects, the amino acid sequences of the at least two distinct RBD domains other than the amino acid sequences of their respective RBM domains are collectively at least 70% identical by amino acid composition to the amino acid sequences of RBD domains sequences other than the amino acid sequences of an RBM from at least two of a clade 1 Sarbecovirus, a clade 2 Sarbecovirus, or a clade 3 Sarbecovirus.

Also provided herein is a composition for delivery of an antigen expression system, comprising: the antigen expression system, wherein the antigen expression system comprises at least two distinct coronavirus receptor binding domain (RBD) derived nucleic acid sequences encoding at least two distinct RBD domains, respectively, and wherein the at least two distinct RBD domains are at least 70% identical by amino acid composition to RBD domains from at least two of a sarbecovirus RBD derived nucleic acid sequence, a merbecoviorus RBD derived nucleic acid sequence, an embecovirus RBD derived nucleic acid sequence, and combinations thereof.

In some aspects, the at least two distinct RBD domains comprises between 2-4, between 2-5, between 2-6, between 2-7, between 2-8, between 2-9, between 2-10, between 2-11, between 2-12, between 2-13, between 2-14, between 2-15, between 2-16, between 2-17, between 2-18, between 2-19, or between 2-20 distinct RBD domains. In some aspects, the at least two distinct RBD domains comprises between 3-8, between 4-8, between 3-8, between 4-8, between 3-9, between 4-9, between 3-10, or between 4-10 distinct RBD domains.

In some aspects, the at least two distinct coronavirus RBD derived nucleic acid sequences are encoded by a single polynucleotide sequence. In some aspects, the at least two distinct coronavirus RBD derived nucleic acid sequences are encoded by a single antigen cassette. In some aspects, the at least two distinct coronavirus RBD derived nucleic acid sequences are each encoded by separate polynucleotide sequences. In some aspects, the at least two distinct coronavirus RBD derived nucleic acid sequences are each encoded by separate antigen cassettes. In some aspects, the separate antigen cassettes are each encoded by separate vectors.

In some aspects, each of the at least two distinct coronavirus RBD derived nucleic acid sequences further comprises a distinct trimerization domain derived nucleic acid sequence. In some aspects, each of the at least two distinct coronavirus RBD derived nucleic acid sequences comprises the same RBD trimerization domain derived nucleic acid sequence. In some aspects, the RBD trimerization domain is selected from the group consisting of: a T4 trimerization domain, a MTQ trimerization domain; a GCN4 trimerization domain, and combinations thereof. In some aspects, each of the at least two distinct coronavirus RBD derived nucleic acid sequences comprises a T4 trimerization domain derived nucleic acid sequence. In some aspects, the at least two distinct coronavirus RBD derived nucleic acid sequences comprises each independently comprise a T4 trimerization domain, a MTQ trimerization domain; a GCN4 trimerization domain.

In some aspects, the coronavirus RBD derived nucleic acid sequence encodes a full-length RBD domain. In some aspects, the coronavirus RBD derived nucleic acid sequence encodes a RBD domain (a) lacking a receptor-binding motif (RBM) domain, (b) comprising an RBM domain that is not derived from the coronavirus the remainder of the RBD is derived from, or (c) comprising RBD sequences that are not derived from the coronavirus the RBM domain is derived from. In some aspects, the coronavirus RBD derived nucleic acid sequence encodes only an RBM domain of the corresponding RBD domain. In some aspects, the at least two distinct coronavirus RBD derived nucleic acid sequences are linked directly to one another. In some aspects, the at least two distinct coronavirus RBD derived nucleic acid sequences are concatenated such that the sequences are capable of being expressed as a single mRNA. In some aspects, the at least two distinct coronavirus RBD derived nucleic acid sequences are linked together with a peptide-linker encoding nucleic acid sequence. In some aspects, the peptide-linker encoding nucleic acid sequence encodes a 2A ribosome skipping sequence element, optionally selected from the group consisting of: a E2A ribosome skipping sequence element, a P2A ribosome skipping sequence element, a F2A ribosome skipping sequence element, a T2A sequence ribosome skipping sequence element, and combinations thereof. In some aspects, the peptide-linker encoding nucleic acid sequence encodes a cleavable peptide linker, optionally selected from: a TEV cleavage site, a furin cleavage site, and combinations thereof. In some aspects, the peptide-linker encoding nucleic acid sequence encodes a T2A sequence ribosome skipping sequence element and a furin cleavage site.

In some aspects, each of the at least two distinct coronavirus RBD derived nucleic acid sequences further comprises a signal-peptide encoding nucleic acid sequence. In some aspects, the signal-peptide comprises a coronavirus derived signal-peptide. In some aspects, the signal-peptide comprises a SARS-COV-2 derived signal-peptide.

In some aspects, one or more of the at least two distinct coronavirus RBD derived nucleic acid sequences are sequence optimized.

In some aspects, the at least two distinct coronavirus RBD derived nucleic acid sequences are operably linked to a promoter. In some aspects, the at least two distinct coronavirus RBD derived nucleic acid sequences are operably linked to a single promoter. In some aspects, each of the at least two distinct coronavirus RBD derived nucleic acid sequences are operably linked to a separate promoter. In some aspects, the promoter comprises a subgenomic promoter sequence, optionally wherein the subgenomic promoter sequence comprises an alphavirus derived subgenomic promoter sequence.

In some aspects, each of the at least two distinct coronavirus RBD derived nucleic acid sequences each separately encodes a peptide of the following format: signal peptide-RBD-trimerization domain. In some aspects, the at least two distinct coronavirus RBD derived nucleic acid sequences encodes a concatenated peptide of the following format: signal peptide-first RBD-first trimerization domain-signal peptide-second RBD-second trimerization domain-signal peptide-third RBD-third trimerization domain. In some aspects, the at least two distinct coronavirus RBD derived nucleic acid sequences encodes a concatenated peptide of the following format: signal peptide-first RBD-first trimerization domain-T2A-Furin-signal peptide-second RBD-second trimerization domain-T2A-Furin-signal peptide-third RBD-third trimerization domain.

In some aspects, at least one of the at least two distinct RBD domains comprises a SARS-COV-2 SPIKE protein. In some aspects, the SARS-COV-2 SPIKE protein is encoded by a SARS-COV-2 SPIKE derived nucleic acid sequence comprising: a) a SARS-COV-2 Spike protein comprising a Spike polypeptide sequence as set forth in SEQ ID NO:59 or an epitope-containing fragment thereof, optionally wherein the Spike polypeptide comprises a D614G mutation with reference to SEQ ID NO:59, and optionally wherein the Spike polypeptide is encoded by the nucleotide sequence shown in SEQ ID NO:79, SEQ ID NO:83, SEQ ID NO:85, or SEQ ID NO:87; or b) a SARS-COV-2 modified Spike protein comprising a mutation selected from the group consisting of: a Spike R682 mutation, a Spike R815 mutation, a Spike K986P mutation, a Spike V987P mutation, and combinations thereof with reference to the Spike polypeptide sequence as set forth in SEQ ID NO:59, and optionally wherein the modified Spike protein comprises a polypeptide sequence as set forth in SEQ ID NO:60 or SEQ ID NO:90 or an epitope-containing fragment thereof.

In some aspects, the antigen expression system comprises at least one coronavirus derived nucleic acid sequence encoding an immunogenic polypeptide distinct from the at least two distinct RBD domains. In some aspects, the at least one coronavirus derived nucleic acid sequence comprises a beta coronavirus derived nucleic acid sequence.

In some aspects, the antigen expression system comprises at least one SARS-COV-2 derived nucleic acid sequence encoding an immunogenic polypeptide. In some aspects, the antigen cassette comprises at least one SARS-COV-2 derived nucleic acid sequence encoding an immunogenic polypeptide. In some aspects, the at least one SARS-COV-2 derived nucleic acid sequence comprises a SARS-COV-2 SPIKE derived nucleic acid sequence. In some aspects, the SARS-COV-2 SPIKE derived nucleic acid sequence comprises: a) a SARS-COV-2 Spike protein comprising a Spike polypeptide sequence as set forth in SEQ ID NO:59 or an epitope-containing fragment thereof, optionally wherein the Spike polypeptide comprises a D614G mutation with reference to SEQ ID NO:59, and optionally wherein the Spike polypeptide is encoded by the nucleotide sequence shown in SEQ ID NO:79, SEQ ID NO:83, SEQ ID NO:85, or SEQ ID NO:87; or b) a SARS-COV-2 modified Spike protein comprising a mutation selected from the group consisting of: a Spike R682 mutation, a Spike R815 mutation, a Spike K986P mutation, a Spike V987P mutation, and combinations thereof with reference to the Spike polypeptide sequence as set forth in SEQ ID NO:59, and optionally wherein the modified Spike protein comprises a polypeptide sequence as set forth in SEQ ID NO:60 or SEQ ID NO:90 or an epitope-containing fragment thereof.

In some aspects, the at least one coronavirus derived nucleic acid sequence and/or SARS-COV-2 derived nucleic acid sequence comprises an MHC class I epitope encoding sequence. In some aspects, the at least one coronavirus derived nucleic acid sequence, SARS-CoV-2 derived nucleic acid sequence, and/or MHC class I epitope encoding sequence comprises:
at least one MHC class I epitope comprising a polypeptide sequence as set forth in Table A,
at least one MHC class II epitope comprising a polypeptide sequence as set forth in Table B,
at least one MHC class I epitope comprising a polypeptide sequence as set forth in Table C, optionally wherein the at least one MHC I epitope is present in a concatenated polypeptide sequence as set forth in SEQ ID NO:57 or SEQ ID NO:58,
at least one polypeptide sequence as set forth in Table 7, or an epitope-containing fragment thereof, optionally wherein the at least one polypeptide sequence is present in a concatenated polypeptide sequence as set forth in SEQ ID NO:92,
at least one polypeptide sequence as set forth in Table 9A, Table 9B, or Table 9C, or an epitope-containing fragment thereof, optionally wherein the at least one polypeptide sequence is present in a concatenated polypeptide comprising each of the sequences set forth in Table 9A, Table 9B, or Table 9C, optionally wherein the concatenated polypeptide comprises the order of sequences set forth in Table 9A, Table 9B, or Table 9C,
at least one MHC class I epitope comprising a polypeptide sequence as set forth in Table A and/or Table C or MHC class II epitope comprising a polypeptide sequence as set forth in Table B, wherein the encoded SARS-COV-2 immunogenic polypeptide is conserved between SARS-COV-2 and a Coronavirus species and/or sub-species other than SARS-COV-2, optionally wherein the Coronavirus species and/or sub-species other than SARS-COV-2 is Severe acute respiratory syndrome (SARS) and/or Middle East respiratory syndrome (MERS),
one or more validated epitopes and/or at least 4, 5, 6, or 7 predicted epitopes, wherein at least 85%, 90%, or 95% of a population carries at least one HLA validated to present at least one of the one or more validated epitopes and/or at least one HLA predicted to present each of the at least 4, 5, 6, or 7 predicted epitopes,
a SARS-COV-2 Spike protein comprising a Spike polypeptide sequence as set forth in SEQ ID NO:59 or an epitope-containing fragment thereof, optionally wherein the Spike polypeptide comprises a D614G mutation with reference to SEQ ID NO:59, and optionally wherein the Spike polypeptide is encoded by the nucleotide sequence shown in SEQ ID NO:79, SEQ ID NO:83, SEQ ID NO:85, or SEQ ID NO:87,
a SARS-COV-2 modified Spike protein comprising a mutation selected from the group consisting of: a Spike R682 mutation, a Spike R815 mutation, a Spike K986P mutation, a Spike V987P mutation, and combinations thereof with reference to the Spike polypeptide sequence as set forth in SEQ ID NO:59, and optionally wherein the modified Spike protein comprises a polypeptide sequence as set forth in SEQ ID NO:60 or SEQ ID NO:90 or an epitope-containing fragment thereof,
a SARS-COV-2 Membrane protein comprising a Membrane polypeptide sequence as set forth in SEQ ID NO:61 or an epitope-containing fragment thereof,
a SARS-COV-2 Nucleocapsid protein comprising a Nucleocapsid polypeptide sequence as set forth in SEQ ID NO:62 or an epitope-containing fragment thereof,
a SARS-COV-2 Envelope protein comprising an Envelope polypeptide sequence as set forth in SEQ ID NO:63 or an epitope-containing fragment thereof,
a variant of any of the above comprising a mutation found in 1% or greater of SARS-CoV-2 subtypes, optionally wherein the variant comprises a SARS-COV-2 variant shown in Table 1, and/or optionally wherein the variant comprises a SARS-COV-2 variant Spike protein comprising a Spike D614G mutation with reference to the Spike polypeptide sequence as set forth in SEQ ID NO:59, a SARS-COV-2 variant Spike protein corresponding to a B.1.351 SARS-COV-2 isolate optionally comprising the Spike polypeptide sequence as set forth in SEQ ID NO: 112, or a SARS-COV-2 variant Spike protein corresponding to a B.1.1.7 SARS-COV-2 isolate optionally comprising the Spike polypeptide sequence as set forth in SEQ ID NO:110,
at least one polypeptide sequence as set forth in Table 16A, Table 16B, Table 16C, or Table 16D, or an epitope-containing fragment thereof, optionally wherein the at least one polypeptide sequence is present in a concatenated polypeptide comprising each of the sequences set forth in Table 16A, Table 16B, Table 16C, or Table 16D, optionally wherein the concatenated polypeptide comprises the order of sequences set forth in Table 16A, Table 16B, Table 16C, or Table 16D,
or combinations thereof.

In some aspects, the MHC class I epitope encoding sequence comprises at least one polypeptide sequence as set forth in Table 16A, Table 16B, Table 16C, or Table 16D, or an epitope-containing fragment thereof, optionally wherein the at least one polypeptide sequence is present in a concatenated polypeptide comprising each of the sequences set forth in Table 16A, Table 16B, Table 16C, or Table 16D, optionally wherein the concatenated polypeptide comprises the order of sequences set forth in Table 16A, Table 16B, Table 16C, or Table 16D.

In some aspects, the at least one coronavirus derived nucleic acid sequence, SARS-CoV-2 derived nucleic acid sequence, and/or MHC class I epitope encoding sequence is selected from the group consisting of: a Spike protein, a Membrane protein, a Nucleocapsid protein, an Envelope protein, a replicase orf1a and orf1b protein, and combinations thereof.

In some aspects, the at least one coronavirus derived nucleic acid sequence is encoded on a separate vector distinct from the vector or vectors encoding the at least two distinct coronavirus RBD derived nucleic acid sequences, optionally wherein the encoded immunogenic polypeptide distinct from the at least two distinct coronavirus RBD comprises a MHC class I epitope encoding sequence comprising at least one polypeptide sequence as set forth in Table 16A, Table 16B, Table 16C, or Table 16D, or an epitope-containing fragment thereof, optionally wherein the at least one polypeptide sequence is present in a concatenated polypeptide comprising each of the sequences set forth in Table 16A, Table 16B, Table 16C, or Table 16D, optionally wherein the concatenated polypeptide comprises the order of sequences set forth in Table 16A, Table 16B, Table 16C, or Table 16D

In some aspects, following administration to a subject, the composition is capable of stimulating an immune response against multiple coronaviruses. In some aspects, following administration to a subject, the composition is capable of stimulating an immune response against at least each of the coronaviruses from which the at least two distinct coronavirus RBD derived nucleic acid sequences were derived. In some aspects, following administration to a subject, the composition is capable of stimulating an immune response against a coronavirus distinct from the coronaviruses from which the at least two distinct coronavirus RBD derived nucleic acid sequences were derived. In some aspects, the composition is capable of stimulating neutralizing antibody production. In some aspects, the composition wherein stimulating neutralizing antibody production comprises a neutralizing antibody titer having an NT50 value calculated as a minimum dilution of sera from the immunized subject that neutralizes a coronavirus by 50%. In some aspects, stimulating neutralizing antibody production comprises a minimum neutralizing antibody titer for complete neutralization of the coronavirus. In some aspects, the composition is capable of stimulating antibody production, wherein the antibodies produced are capable of antibody-mediated viral clearance, optionally wherein the antibody-mediated viral clearance comprises Fc-mediated viral clearance.

In some aspects, the antigen expression system comprises one or more vectors, the one or more vectors comprising: (a) a vector backbone, wherein the vector backbone comprises a chimpanzee adenovirus vector, optionally wherein the chimpanzee adenovirus vector is a ChAdV68 vector, or an alphavirus vector, optionally wherein the alphavirus vector is a Venezuelan equine encephalitis virus vector; and (b) a cassette encoding the at least two distinct RBD domains, optionally wherein the cassette is integrated between a native promoter nucleotide sequence native to the vector backbone and a poly(A) sequence, optionally wherein the poly(A) sequence is native to the vector backbone.

In some aspects, the antigen expression system comprises one or more vectors, the one or more vectors comprising a vector backbone derived from a Venezuelan equine encephalitis virus.

Also provided for herein is a composition for delivery of an antigen expression system, comprising: the antigen expression system, wherein the antigen expression system comprises: (a) optionally, one or more vectors, the one or more vectors comprising: a vector backbone, wherein the backbone comprises: (i) at least one promoter nucleotide sequence, and (ii) at least one polyadenylation (poly(A)) sequence; and (b) an antigen cassette, optionally wherein the antigen cassette is inserted into the vector backbone when present, and wherein the antigen cassette comprises at least one SARS-COV-2 derived nucleic acid sequence encoding an immunogenic polypeptide, wherein the immunogenic polypeptide comprises at least one polypeptide sequence as set forth in Table 16A, Table 16B, Table 16C, or Table 16D, or an epitope-containing fragment thereof, optionally wherein the at least one polypeptide sequence is present in a concatenated polypeptide comprising each of the sequences set forth in Table 16A, Table 16B, Table 16C, or Table 16D, optionally wherein the concatenated polypeptide comprises the order of sequences set forth in Table 16A, Table 16B, Table 16C, or Table 16D.

In some aspects, the MHC class I epitope encoding sequence comprises at least one polypeptide sequence as set forth in Table 16A, Table 16B, Table 16C, or Table 16D, or an epitope-containing fragment thereof, optionally wherein the at least one polypeptide sequence is present in a concatenated polypeptide comprising each of the sequences set forth in Table 16A, Table 16B, Table 16C, or Table 16D, optionally wherein the concatenated polypeptide comprises the order of sequences set forth in Table 16A, Table 16B, Table 16C, or Table 16D.

Also provided for is a method for treating a coronavirus infection or preventing a coronavirus infection in a subject, the method comprising administering to the subject any one of the compositions described herein.

Also provided for herein is a method for inducing an immune response in a subject, the method comprising administering to the subject any one of the compositions described herein.

In some aspects, the method comprises a homologous prime/boost strategy. In some aspects, the method comprises a heterologous prime/boost strategy, optionally wherein the heterologous prime/boost strategy comprises (a) an identical antigen cassette encoded by different vaccine platforms, (b) different antigen cassettes encoded by the same vaccine platform, and/or (c) different antigen cassettes encoded by different vaccine platforms.

In some aspects, the method comprises administering a vector or vectors encoding the at least two distinct coronavirus RBD derived nucleic acid sequences and administering a vector or vectors encoding at least one coronavirus derived nucleic acid sequence encoding an immunogenic polypeptide distinct from the at least two distinct RBD domains.

In some aspects, the vector or vectors encoding the at least two distinct coronavirus RBD derived nucleic acid sequences and the vector or vectors encoding at least one coronavirus derived nucleic acid sequence are co-formulated. In some aspects, the vector or vectors encoding the at least two distinct coronavirus RBD derived nucleic acid sequences and the vector or vectors encoding at least one coronavirus derived nucleic acid sequence are administered separately. In some aspects, the vector or vectors encoding the at least two distinct coronavirus RBD derived nucleic acid sequences and the vector or vectors encoding at least one coronavirus derived nucleic acid sequence are administered concurrently. In some aspects, the at least one coronavirus derived nucleic acid sequence comprises:
at least one MHC class I epitope comprising a polypeptide sequence as set forth in Table A,
at least one MHC class II epitope comprising a polypeptide sequence as set forth in Table B,
at least one MHC class I epitope comprising a polypeptide sequence as set forth in Table C, optionally wherein the at least one MHC I epitope is present in a concatenated polypeptide sequence as set forth in SEQ ID NO:57 or SEQ ID NO:58,
at least one polypeptide sequence as set forth in Table 7, or an epitope-containing fragment thereof, optionally wherein the at least one polypeptide sequence is present in a concatenated polypeptide sequence as set forth in SEQ ID NO:92,
at least one polypeptide sequence as set forth in Table 9A, Table 9B, or Table 9C, or an epitope-containing fragment thereof, optionally wherein the at least one polypeptide sequence is present in a concatenated polypeptide comprising each of the sequences set forth in Table 9A, Table 9B, or Table 9C, optionally wherein the concatenated polypeptide comprises the order of sequences set forth in Table 9A, Table 9B, or Table 9C,
at least one MHC class I epitope comprising a polypeptide sequence as set forth in Table A and/or Table C or MHC class II epitope comprising a polypeptide sequence as set forth in Table B, wherein the encoded SARS-COV-2 immunogenic polypeptide is conserved between SARS-CoV-2 and a Coronavirus species and/or sub-species other than SARS-COV-2, optionally wherein the Coronavirus species and/or sub-species other than SARS-COV-2 is Severe acute respiratory syndrome (SARS) and/or Middle East respiratory syndrome (MERS),
one or more validated epitopes and/or at least 4, 5, 6, or 7 predicted epitopes, wherein at least 85%, 90%, or 95% of a population carries at least one HLA validated to present at least one of the one or more validated epitopes and/or at least one HLA predicted to present each of the at least 4, 5, 6, or 7 predicted epitopes,
a SARS-COV-2 Spike protein comprising a Spike polypeptide sequence as set forth in SEQ ID NO:59 or an epitope-containing fragment thereof, optionally wherein the Spike polypeptide comprises a D614G mutation with reference to SEQ ID NO:59, and optionally wherein the Spike polypeptide is encoded by the nucleotide sequence shown in SEQ ID NO:79, SEQ ID NO:83, SEQ ID NO:85, or SEQ ID NO:87,
a SARS-COV-2 modified Spike protein comprising a mutation selected from the group consisting of: a Spike R682 mutation, a Spike R815 mutation, a Spike K986P mutation, a Spike V987P mutation, and combinations thereof with reference to the Spike polypeptide sequence as set forth in SEQ ID NO:59, and optionally wherein the modified Spike protein comprises a polypeptide sequence as set forth in SEQ ID NO:60 or SEQ ID NO:90 or an epitope-containing fragment thereof,
a SARS-COV-2 Membrane protein comprising a Membrane polypeptide sequence as set forth in SEQ ID NO:61 or an epitope-containing fragment thereof,
a SARS-COV-2 Nucleocapsid protein comprising a Nucleocapsid polypeptide sequence as set forth in SEQ ID NO:62 or an epitope-containing fragment thereof,
a SARS-COV-2 Envelope protein comprising an Envelope polypeptide sequence as set forth in SEQ ID NO:63 or an epitope-containing fragment thereof,
a variant of any of the above comprising a mutation found in 1% or greater of SARS-COV-2 subtypes, optionally wherein the variant comprises a SARS-COV-2 variant shown in Table 1, and/or optionally wherein the variant comprises a SARS-COV-2 variant Spike protein comprising a Spike D614G mutation with reference to the Spike polypeptide sequence as set forth in SEQ ID NO:59, a SARS-COV-2 variant Spike protein corresponding to a B.1.351 SARS-COV-2 isolate optionally comprising the Spike polypeptide sequence as set forth in SEQ ID NO:112, or a SARS-COV-2 variant Spike protein corresponding to a B.1.1.7 SARS-COV-2 isolate optionally comprising the Spike polypeptide sequence as set forth in SEQ ID NO:110,
at least one polypeptide sequence as set forth in Table 16A, Table 16B, Table 16C, or Table 16D, or an epitope-containing fragment thereof, optionally wherein the at least one polypeptide sequence is present in a concatenated polypeptide comprising each of the sequences set forth in Table 16A, Table 16B, Table 16C, or Table 16D, optionally wherein the concatenated polypeptide comprises the order of sequences set forth in Table 16A, Table 16B, Table 16C, or Table 16D,
or combinations thereof.

In some aspects, the at least one coronavirus derived nucleic acid sequence comprises a MHC class I epitope encoding sequence comprising at least one polypeptide sequence as set forth in Table 16A, Table 16B, Table 16C, or Table 16D, or an epitope-containing fragment thereof, optionally wherein the at least one polypeptide sequence is present in a concatenated polypeptide comprising each of the sequences set forth in Table 16A, Table 16B, Table 16C, or Table 16D, optionally wherein the concatenated polypeptide comprises the order of sequences set forth in Table 16A, Table 16B, Table 16C, or Table 16D.

In some aspects, the composition further comprises a nanoparticulate delivery vehicle. In some aspects, the nanoparticulate delivery vehicle is a lipid nanoparticle (LNP). In some aspects, the LNP comprises ionizable amino lipids. In some aspects, the ionizable amino lipids comprise MC3-like (dilinoleylmethyl-4-dimethylaminobutyrate) molecules. In some aspects, the nanoparticulate delivery vehicle encapsulates the antigen expression system.

In some aspects, the one or more vectors comprise one or more+−stranded RNA In some aspects, the one or more+−stranded RNA vectors comprise a 5′ 7-methylguanosine (m7g) cap. In some aspects, the one or more+−stranded RNA vectors are produced by in vitro transcription. In some aspects, the one or more vectors are self-replicating within a mammalian cell.

In some aspects, the backbone comprises at least one nucleotide sequence of an Aura virus, a Fort Morgan virus, a Venezuelan equine encephalitis virus, a Ross River virus, a Semliki Forest virus, a Sindbis virus, or a Mayaro virus. In some aspects, the backbone comprises at least one nucleotide sequence of a Venezuelan equine encephalitis virus. In some aspects, the backbone comprises at least sequences for nonstructural protein-mediated amplification, a 26S promoter sequence, a poly(A) sequence, a nonstructural protein 1 (nsP1) gene, a nsP2 gene, a nsP3 gene, and a nsP4 gene encoded by the nucleotide sequence of the Aura virus, the Fort Morgan virus, the Venezuelan equine encephalitis virus, the Ross River virus, the Semliki Forest virus, the Sindbis virus, or the Mayaro virus. In some aspects, the backbone comprises at least sequences for nonstructural protein-mediated amplification, a 26S promoter sequence, and a poly(A) sequence encoded by the nucleotide sequence of the Aura virus, the Fort Morgan virus, the Venezuelan equine encephalitis virus, the Ross River virus, the Semliki Forest virus, the Sindbis virus, or the Mayaro virus. In some aspects, sequences for nonstructural protein-mediated amplification are selected from the group consisting of: an alphavirus 5′ UTR, a 51-nt CSE, a 24-nt CSE, a 26S subgenomic promoter sequence, a 19-nt CSE, an alphavirus 3′ UTR, or combinations thereof. In some aspects, the backbone does not encode structural virion proteins capsid, E2 and E1. In some aspects, the antigen cassette is inserted in place of structural virion proteins within the nucleotide sequence of the Aura virus, the Fort Morgan virus, the Venezuelan equine encephalitis virus, the Ross River virus, the Semliki Forest virus, the Sindbis virus, or the Mayaro virus. In some aspects, the Venezuelan equine encephalitis virus comprises the sequence of SEQ ID NO:3 or SEQ ID NO:5. In some aspects, the Venezuelan equine encephalitis virus comprises the sequence of SEQ ID NO:3 or SEQ ID NO:5 further comprising a deletion between base pair 7544 and 11175. In some aspects, the backbone comprises the sequence set forth in SEQ ID NO:6 or SEQ ID NO:7. In some aspects, the antigen cassette is inserted at position 7544 to replace the deletion between base pairs 7544 and 11175 as set forth in the sequence of SEQ ID NO:3 or SEQ ID NO:5. In some aspects, the insertion of the antigen cassette provides for transcription of a polycistronic RNA comprising the nsP1-4 genes and the at least one coronavirus derived nucleic acid sequence, wherein the nsP1-4 genes and the at least one coronavirus derived nucleic acid sequence are in separate open reading frames. In some aspects, the at least one promoter nucleotide sequence is the native 26S promoter nucleotide sequence encoded by the backbone.

In some aspects, the backbone comprises at least one nucleotide sequence of a chimpanzee adenovirus vector, optionally wherein the chimpanzee adenovirus vector is a ChAdV68 vector. In some aspects, the ChAdV68 vector backbone comprises the sequence set forth in SEQ ID NO:1. In some aspects, the ChAdV68 vector backbone comprises the sequence set forth in SEQ ID NO:1, except that the sequence is fully deleted or functionally deleted in at least one gene selected from the group consisting of the chimpanzee adenovirus E1A, E1B, E2A, E2B, E3, E4, L1, L2, L3, L4, and L5 genes of the sequence set forth in SEQ ID NO:1, optionally wherein the sequence is fully deleted or functionally deleted in: (1) E1A and E1B; (2) E1A, E1B, and E3; or (3) E1A, E1B, E3, and E4 of the sequence set forth in SEQ ID NO: 1. In some aspects, the ChAdV68 vector backbone comprises a gene or regulatory sequence obtained from the sequence of SEQ ID NO:1, optionally wherein the gene is selected from the group consisting of the chimpanzee adenovirus inverted terminal repeat (ITR), E1A, E1B, E2A, E2B, E3, E4, L1, L2, L3, L4, and L5 genes of the sequence set forth in SEQ ID NO: 1. In some aspects, the ChAdV68 vector backbone comprises a partially deleted E4 gene comprising a deleted or partially-deleted E4orf2 region and a deleted or partially-deleted E4orf3 region, and optionally a deleted or partially-deleted E4orf4 region. In some aspects, the ChAdV68 vector backbone comprises at least nucleotides 2 to 36,518 of the sequence set forth in SEQ ID NO:1 and further comprising: (1) an E1 deletion of at least nucleotides 577 to 3403 of the sequence shown in SEQ ID NO: 1, (2) an E3 deletion of at least nucleotides 27,125 to 31,825 of the sequence shown in SEQ ID NO:1, and (3) an E4 deletion of at least nucleotides 34,916 to 35,642 of the sequence shown in SEQ ID NO:1; optionally wherein the antigen cassette is inserted within the E1 deletion. In some aspects, the ChAdV68 vector backbone comprises the sequence set forth in SEQ ID NO:75, optionally wherein the antigen cassette is inserted within the E1 deletion. In some aspects, the ChAdV68 vector backbone comprises one or more deletions between base pair number 577 and 3403 or between base pair 456 and 3014, and optionally wherein the vector further comprises one or more deletions between base pair 27,125 and 31,825 or between base pair 27,816 and 31,333 of the sequence set forth in SEQ ID NO:1. In some aspects, the ChAdV68 vector backbone comprises one or more deletions between base pair number 3957 and 10346, base pair number 21787 and 23370, and base pair number 33486 and 36193 of the sequence set forth in SEQ ID NO: 1. In some aspects, the wherein the cassette is inserted in the ChAdV backbone at the E1 region, E3 region, and/or any deleted AdV region that allows incorporation of the cassette. In some aspects, the ChAdV backbone is generated from one of a first generation, a second generation, or a helper-dependent adenoviral vector

In some aspects, the at least one promoter nucleotide sequence is selected from the group consisting of: a CMV, a SV40, an EF-1, a RSV, a PGK, a HSA, a MCK, and a EBV promoter sequence. In some aspects, the at least one promoter nucleotide sequence is a CMV promoter sequence. In some aspects, the at least one promoter nucleotide sequence is an exogenous RNA promoter. In some aspects, the second promoter nucleotide sequence is a 26S promoter nucleotide sequence or a CMV promoter nucleotide sequence. In some aspects, the second promoter nucleotide sequence comprises multiple 26S promoter nucleotide sequences or multiple CMV promoter nucleotide sequences, wherein each 26S promoter nucleotide sequence or CMV promoter nucleotide sequence provides for transcription of one or more of the separate open reading frames.

In some aspects, a MHC class I or MHC class II epitope-encoding coronavirus derived nucleic acid sequence is selected by performing the steps of: (a) obtaining at least one of exome, transcriptome, or whole genome coronavirus nucleotide sequencing data from a coronavirus virus or coronavirus infected cell, wherein the coronavirus nucleotide sequencing data is used to obtain data representing peptide sequences of each of a set of antigens; (b) inputting the peptide sequence of each antigen into a presentation model to generate a set of numerical likelihoods that each of the antigens is presented by one or more of the MHC alleles on a coronavirus infected cell surface, the set of numerical likelihoods having been identified at least based on received mass spectrometry data; and (c) selecting a subset of the set of antigens based on the set of numerical likelihoods to generate a set of selected antigens which are used to generate the MHC class I or MHC class II epitope-encoding coronavirus derived nucleic acid sequence.

In some aspects, each MHC class I or MHC class II epitope-encoding coronavirus derived nucleic acid sequences is selected by performing the steps of: (a) obtaining at least one of exome, transcriptome, or whole genome coronavirus nucleotide sequencing data from a coronavirus virus or coronavirus infected cell, wherein the coronavirus nucleotide sequencing data is used to obtain data representing peptide sequences of each of a set of antigens; (b) inputting the peptide sequence of each antigen into a presentation model to generate a set of numerical likelihoods that each of the antigens is presented by one or more of the MHC alleles on a coronavirus infected cell surface, the set of numerical likelihoods having been identified at least based on received mass spectrometry data; and (c) selecting a subset of the set of antigens based on the set of numerical likelihoods to generate a set of selected antigens which are used to generate the at least 18 coronavirus derived nucleic acid sequences. In some aspects, a number of the set of selected antigens is 2-20. In some aspects, the presentation model represents dependence between: (a) presence of a pair of a particular one of the MHC alleles and a particular amino acid at a particular position of a peptide sequence; and (b) likelihood of presentation on a coronavirus infected cell surface, by the particular one of the MHC alleles of the pair, of such a peptide sequence comprising the particular amino acid at the particular position. In some aspects, selecting the set of selected antigens comprises selecting antigens that have an increased likelihood of being presented on a coronavirus infected cell surface relative to unselected antigens based on the presentation model, optionally wherein the selected antigens have been validated as being presented by one or more specific HLA alleles. In some aspects, selecting the set of selected antigens comprises selecting antigens that have an increased likelihood of being capable of inducing a coronavirus specific immune response in the subject relative to unselected antigens based on the presentation model. In some aspects, selecting the set of selected antigens comprises selecting antigens that have an increased likelihood of being capable of being presented to naïve T cells by professional antigen presenting cells (APCs) relative to unselected antigens based on the presentation model, optionally wherein the APC is a dendritic cell (DC). In some aspects, selecting the set of selected antigens comprises selecting antigens that have a decreased likelihood of being subject to inhibition via central or peripheral tolerance relative to unselected antigens based on the presentation model. In some aspects, selecting the set of selected antigens comprises selecting antigens that have a decreased likelihood of being capable of inducing an autoimmune response to normal tissue in the subject relative to unselected antigens based on the presentation model. In some aspects, exome or transcriptome coronavirus nucleotide sequencing data is obtained by performing sequencing on a coronavirus virus or coronavirus infected tissue or cell. In some aspects, the sequencing is next generation sequencing (NGS) or any massively parallel sequencing approach.

In some aspects, the antigen cassette comprises junctional epitope sequences formed by adjacent sequences in the antigen cassette. In some aspects, at least one or each junctional epitope sequence has an affinity of greater than 500 nM for MHC. In some aspects, each junctional epitope sequence is non-self.

In some aspects, each of the MHC class I and/or MHC class II epitopes is predicted or validated to be capable of presentation by at least one HLA allele present in at least 5% of a population. In some aspects, each of the MHC class I and/or MHC class II epitopes is predicted or validated to be capable of presentation by at least one HLA allele, wherein each antigen/HLA pair has an antigen/HLA prevalence of at least 0.01% in a population. In some aspects, each of the MHC class I and/or MHC class II epitopes is predicted or validated to be capable of presentation by at least one HLA allele, wherein each antigen/HLA pair has an antigen/HLA prevalence of at least 0.1% in a population.

In some aspects, the antigen cassette does not encode a non-therapeutic MHC class I or class II epitope nucleic acid sequence comprising a translated, wild-type nucleic acid sequence, wherein the non-therapeutic epitope is predicted to be displayed on an MHC allele of the subject. In some aspects, the non-therapeutic predicted MHC class I or class II epitope sequence is a junctional epitope sequence formed by adjacent sequences in the antigen cassette.

In some aspects, the prediction is based on presentation likelihoods generated by inputting sequences of the non-therapeutic epitopes into a presentation model. In some aspects, an order of the at least one coronavirus derived nucleic acid sequences in the antigen cassette is determined by a series of steps comprising: (a) generating a set of candidate antigen cassette sequences corresponding to different orders of the at least one coronavirus derived nucleic acid sequences; (b) determining, for each candidate antigen cassette sequence, a presentation score based on presentation of non-therapeutic epitopes in the candidate antigen cassette sequence; and (c) selecting a candidate cassette sequence associated with a presentation score below a predetermined threshold as the antigen cassette sequence for an antigen vaccine.

Also provided for herein is a pharmaceutical composition any of the compositions provided herein and a pharmaceutically acceptable carrier. In some aspects, the composition further comprises an adjuvant. In some aspects, the composition further comprises an immune modulator. In some aspects, the immune modulator is an anti-CTLA4 antibody or an antigen-binding fragment thereof, an anti-PD-1 antibody or an antigen-binding fragment thereof, an anti-PD-L1 antibody or an antigen-binding fragment thereof, an anti-4-1BB antibody or an antigen-binding fragment thereof, or an anti-OX-40 antibody or an antigen-binding fragment thereof.

Also provided herein is a vector or set of vectors comprising any of the isolated nucleotide sequences or set of isolated nucleotide sequences provided herein.

Also provided herein is an isolated cell comprising any of the isolated nucleotide sequences or set of isolated nucleotide sequences provided herein, optionally wherein the cell is a BHK-21, CHO, HEK293 or variants thereof, 911, HeLa, A549, LP-293, PER.C6, or AE1-2a cell.

Also provided herein is a kit comprising any of the compositions provided herein and instructions for use.

Also provided herein is a method for treating a coronavirus infection or preventing a coronavirus infection in a subject, the method comprising administering to the subject any of the compositions or pharmaceutical compositions provided herein. In some aspects, the coronavirus derived nucleic acid sequence encodes at least one immunogenic polypeptide corresponding to a polypeptide encoded by a coronavirus subtype the subject is infected with or at risk for infection by.

In some aspects, any of the methods described herein comprises a homologous prime/boost strategy. In some aspects, any of the methods described herein comprises a heterologous prime/boost strategy. In some aspects, the heterologous prime/boost strategy comprises an identical antigen cassette encoded by different vaccine platforms. In some aspects, the heterologous prime/boost strategy comprises different antigen cassettes encoded by the same vaccine platform. In some aspects, the heterologous prime/boost strategy comprises different antigen cassettes encoded by different vaccine platforms. In some aspects, the different antigen cassettes comprise a Spike-encoding cassette and a separate T cell epitope encoding cassette. In some aspects, the different antigen cassettes comprise cassettes encoding distinct epitopes and/or antigens derived from different isolates of coronavirus.

Also provided herein is a method for inducing an immune response in a subject, the method comprising administering to the subject any of the compositions or pharmaceutical compositions provided herein. In some aspects, the subject expresses at least one HLA allele predicted or known to present a MHC class I or MHC class II epitope encoded by the at least one coronavirus derived nucleic acid sequence. In some aspects, the subject expresses at least one HLA allele predicted or known to present a MHC class I epitope encoded by the at least one coronavirus derived nucleic acid sequence, and wherein the MHC class I epitope comprises at least one MHC class I epitope comprising a polypeptide sequence as set forth in Table A. In some aspects, the subject express at least one HLA allele predicted or known to present a MHC class II epitope encoded by the at least one coronavirus derived nucleic acid sequence, and wherein the MHC class II epitope comprises at least one MHC class II epitope comprising a polypeptide sequence as set forth in Table B. In some aspects, the composition is administered intramuscularly (IM), intradermally (ID), subcutaneously (SC), or intravenously (IV). In some aspects, the composition is administered intramuscularly

In some aspects, the method further comprises administering to the subject a second vaccine composition. In some aspects, the second vaccine composition is administered prior to the administration of the first composition or pharmaceutical composition. In some aspects, the second vaccine composition is administered subsequent to the administration of any of the compositions or pharmaceutical compositions provided herein. In some aspects, the second vaccine composition is the same as the first composition or pharmaceutical composition administered. In some aspects, the second vaccine composition is different from the first composition or pharmaceutical composition administered. In some aspects, the second vaccine composition comprises a chimpanzee adenovirus vector encoding at least one coronavirus derived nucleic acid sequence. In some aspects, the at least one coronavirus derived nucleic acid sequence encoded by the chimpanzee adenovirus vector is the same as the at least one coronavirus derived nucleic acid sequence of any of the compositions provided herein.

Also provided herein is a method of manufacturing the one or more vectors of any of the above composition claims, the method comprising: (a) obtaining a linearized DNA sequence comprising the backbone and the antigen cassette; (b) in vitro transcribing the linearized DNA sequence by addition of the linearized DNA sequence to an in vitro transcription reaction containing all the necessary components to transcribe the linearized DNA sequence into RNA, optionally further comprising in vitro addition of the m7g cap to the resulting RNA; and (c) isolating the one or more vectors from the in vitro transcription reaction. In some aspects, the linearized DNA sequence is generated by linearizing a DNA plasmid sequence or by amplification using PCR. In some aspects, the DNA plasmid sequence is generated using one of bacterial recombination or full genome DNA synthesis or full genome DNA synthesis with amplification of synthesized DNA in bacterial cells. In some aspects, isolating the one or more vectors from the in vitro transcription reaction involves one or more of phenol chloroform extraction, silica column based purification, or similar RNA purification methods.

Also provided herein is a method of manufacturing the composition of any of the above composition claims for delivery of the antigen expression system, the method comprising: (a) providing components for the nanoparticulate delivery vehicle; (b) providing the antigen expression system; and (c) providing conditions sufficient for the nanoparticulate delivery vehicle and the antigen expression system to produce the composition for delivery of the antigen expression system. In some aspects, the conditions are provided by microfluidic mixing.

Also provided herein is a method of manufacturing an adenovirus vector disclosed herein, the method comprising: obtaining a plasmid sequence comprising the at least one promoter sequence and the antigen cassette; transfecting the plasmid sequence into one or more host cells; and isolating the adenovirus vector from the one or more host cells.

In some aspects, isolating comprises: lysing the host cell to obtain a cell lysate comprising the adenovirus vector; and purifying the adenovirus vector from the cell lysate.

In some aspects, the plasmid sequence is generated using one of bacterial recombination or full genome DNA synthesis or full genome DNA synthesis with amplification of synthesized DNA in bacterial cells. In some aspects, the one or more host cells are at least one of CHO, HEK293 or variants thereof, 911, HeLa, A549, LP-293, PER.C6, and AE1-2a cells. In some aspects, purifying the adenovirus vector from the cell lysate involves one or more of chromatographic separation, centrifugation, virus precipitation, and filtration.

In some aspects, any of the above compositions further comprise a nanoparticulate delivery vehicle. The nanoparticulate delivery vehicle, in some aspects, may be a lipid nanoparticle (LNP). In some aspects, the LNP comprises ionizable amino lipids. In some aspects, the ionizable amino lipids comprise MC3-like (dilinoleylmethyl-4-dimethylaminobutyrate) molecules. In some aspects, the nanoparticulate delivery vehicle encapsulates the antigen expression system.

In some aspects, any of the above compositions further comprise a plurality of LNPs, wherein the LNPs comprise: the antigen expression system; a cationic lipid; a non-cationic lipid; and a conjugated lipid that inhibits aggregation of the LNPs, wherein at least about 95% of the LNPs in the plurality of LNPs either: have a non-lamellar morphology; or are electron-dense.

In some aspects, the non-cationic lipid is a mixture of (1) a phospholipid and (2) cholesterol or a cholesterol derivative.

In some aspects, the conjugated lipid that inhibits aggregation of the LNPs is a polyethyleneglycol (PEG)-lipid conjugate. In some aspects, the PEG-lipid conjugate is selected from the group consisting of: a PEG-diacylglycerol (PEG-DAG) conjugate, a PEG dialkyloxypropyl (PEG-DAA) conjugate, a PEG-phospholipid conjugate, a PEG-ceramide (PEG-Cer) conjugate, and a mixture thereof. In some aspects the PEG-DAA conjugate is a member selected from the group consisting of: a PEG-didecyloxypropyl (C10) conjugate, a PEG-dilauryloxypropyl (C12) conjugate, a PEG-dimyristyloxypropyl (C14) conjugate, a PEG-dipalmityloxypropyl (C16) conjugate, a PEG-distearyloxypropyl (C18) conjugate, and a mixture thereof.

In some aspects, the antigen expression system is fully encapsulated in the LNPs.

In some aspects, the non-lamellar morphology of the LNPs comprises an inverse hexagonal (Hu) or cubic phase structure.

In some aspects, the cationic lipid comprises from about 10 mol % to about 50 mol % of the total lipid present in the LNPs. In some aspects, the cationic lipid comprises from about 20 mol % to about 50 mol % of the total lipid present in the LNPs. In some aspects, the cationic lipid comprises from about 20 mol % to about 40 mol % of the total lipid present in the LNPs.

In some aspects, the non-cationic lipid comprises from about 10 mol % to about 60 mol % of the total lipid present in the LNPs. In some aspects, the non-cationic lipid comprises from about 20 mol % to about 55 mol % of the total lipid present in the LNPs. In some aspects, the non-cationic lipid comprises from about 25 mol % to about 50 mol % of the total lipid present in the LNPs.

In some aspects, the conjugated lipid comprises from about 0.5 mol % to about 20 mol % of the total lipid present in the LNPs. In some aspects, the conjugated lipid comprises from about 2 mol % to about 20 mol % of the total lipid present in the LNPs. In some aspects, the conjugated lipid comprises from about 1.5 mol % to about 18 mol % of the total lipid present in the LNPs.

In some aspects, greater than 95% of the LNPs have a non-lamellar morphology. In some aspects, greater than 95% of the LNPs are electron dense.

In some aspects, any of the above compositions further comprise a plurality of LNPs, wherein the LNPs comprise: a cationic lipid comprising from 50 mol % to 65 mol % of the total lipid present in the LNPs; a conjugated lipid that inhibits aggregation of LNPs comprising from 0.5 mol % to 2 mol % of the total lipid present in the LNPs; and a non-cationic lipid comprising either: a mixture of a phospholipid and cholesterol or a derivative thereof, wherein the phospholipid comprises from 4 mol % to 10 mol % of the total lipid present in the LNPs and the cholesterol or derivative thereof comprises from 30 mol % to 40 mol % of the total lipid present in the LNPs; a mixture of a phospholipid and cholesterol or a derivative thereof, wherein the phospholipid comprises from 3 mol % to 15 mol % of the total lipid present in the LNPs and the cholesterol or derivative thereof comprises from 30 mol % to 40 mol % of the total lipid present in the LNPs; or up to 49.5 mol % of the total lipid present in the LNPs and comprising a mixture of a phospholipid and cholesterol or a derivative thereof, wherein the cholesterol or derivative thereof comprises from 30 mol % to 40 mol % of the total lipid present in the LNPs.

In some aspects, any of the above compositions further comprise a plurality of LNPs, wherein the LNPs comprise: a cationic lipid comprising from 50 mol % to 85 mol % of the total lipid present in the LNPs; a conjugated lipid that inhibits aggregation of LNPs comprising from 0.5 mol % to 2 mol % of the total lipid present in the LNPs; and a non-cationic lipid comprising from 13 mol % to 49.5 mol % of the total lipid present in the LNPs.

In some aspects, the phospholipid comprises dipalmitoylphosphatidylcholine (DPPC), distearoylphosphatidylcholine (DSPC), or a mixture thereof.

In some aspects, the conjugated lipid comprises a polyethyleneglycol (PEG)-lipid conjugate. In some aspects, the PEG-lipid conjugate comprises a PEG-diacylglycerol (PEG-DAG) conjugate, a PEG-dialkyloxypropyl (PEG-DAA) conjugate, or a mixture thereof. In some aspects, the PEG-DAA conjugate comprises a PEG-dimyristyloxypropyl (PEG-DMA) conjugate, a PEG-distearyloxypropyl (PEG-DSA) conjugate, or a mixture thereof. In some aspects, the PEG portion of the conjugate has an average molecular weight of about 2,000 daltons.

In some aspects, the conjugated lipid comprises from 1 mol % to 2 mol % of the total lipid present in the LNPs.

In some aspects, the LNP comprises a compound having a structure of Formula I:

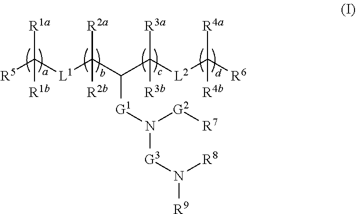

or a pharmaceutically acceptable salt, tautomer, prodrug or stereoisomer thereof, wherein: L¹ and L² are each independently -0(C=0)-, —(C=0)0-, —C(=0)-, -0-, —S(0)ₓ—, —S—S—, —C(=0)S—, —SC(=0)-, —R^{a}C(=0)-, —C(=0)R^{a}—, —R^{a}C(=0)R^{a}—, —OC(=0)R^{a}—, —R^{a}C(=0)0- or a direct bond; G¹ is Ci-C₂ alkylene, —(C=0)-, -0(C=0)-, —SC(=0)-, —R^{a}C(=0)- or a direct bond: —C(=0)-, —(C=0)0-, —C(=0)S—, —C(=0)R^{a}— or a direct bond; G is Ci-C₆ alkylene; R^{a} is H or C1-C12 alkyl; R^{1a} and R^{1b} are, at each occurrence, independently either: (a) H or C₁-C₁₂ alkyl; or (b) R^{1a} is H or C₁-C₁₂ alkyl, and R^{1b} together with the carbon atom to which it is bound is taken together with an adjacent Rib and the carbon atom to which it is bound to form a carbon-carbon double bond; R^{2a} and R^{2b} are, at each occurrence, independently either: (a) H or C₁-C₁₂ alkyl; or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond; R^{3a} and R^{3b} are, at each occurrence, independently either (a): H or C₁-C₁₂ alkyl; or (b) R^{3a} is H or C₁-C₁₂ alkyl, and R^{3b} together with the carbon atom to which it is bound is taken together with an adjacent R and the carbon atom to which it is bound to form a carbon-carbon double bond; R^{4a} and R^{4b} are, at each occurrence, independently either: (a) H or C1-C12 alkyl; or (b) R^{4a} is H or C1-C12 alkyl, and R^{4b} together with the carbon atom to which it is bound is taken together with an adjacent R^{4b} and the carbon atom to which it is bound to form a carbon-carbon double bond; R⁵ and R⁶ are each independently H or methyl; R⁷ is C4-C20 alkyl; R⁸ and R⁹ are each independently C1-C12 alkyl; or R8 and R9, together with the nitrogen atom to which they are attached, form a 5, 6 or 7-membered heterocyclic ring; a, b, c and d are each independently an integer from 1 to 24; and x is 0, 1 or 2.

In some aspects, the LNP comprises a compound having a structure of Formula II:

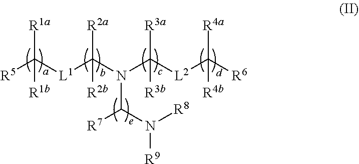

or a pharmaceutically acceptable salt, tautomer, prodrug or stereoisomer thereof, wherein: L¹ and L² are each independently -0(C=0)-, —(C=0)0- or a carbon-carbon double bond; R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond; R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond; R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond; R^{4a} and R^{4b} are, at each occurrence, independently either (a) H or C₁-C₁₂ alkyl, or (b) R^{4a} is H or Ci-C₁₂ alkyl, and R^{4b} together with the carbon atom to which it is bound is taken together with an adjacent R^{4b} and the carbon atom to which it is bound to form a carbon-carbon double bond; R⁵ and R⁶ are each independently methyl or cycloalkyl; R⁷ is, at each occurrence, independently H or C₁-C₁₂ alkyl; R⁸ and R⁹ are each independently unsubstituted C1-C12 alkyl; or R⁸ and R⁹, together with the nitrogen atom to which they are attached, form a 5, 6 or 7-membered heterocyclic ring comprising one nitrogen atom; a and d are each independently an integer from 0 to 24; b and c are each independently an integer from 1 to 24; and e is 1 or 2, provided that: at least one of R^{1a}, R^{2a}, R^{3a} or R^{4a} is C1-C12 alkyl, or at least one of L¹ or L² is -0(C=0)- or —(C=0)0-; and R^{1a} and R^{1b} are not isopropyl when a is 6 or n-butyl when a is 8.

In some aspects, any of the above compositions further comprise one or more excipients comprising a neutral lipid, a steroid, and a polymer conjugated lipid. In some aspects, the neutral lipid comprises at least one of 1,2-Distearoyl-sn-glycero-3-phosphocholine (DSPC), 1,2-Dipalmitoyl-sn-glycero-3-phosphocholine (DPPC), 1,2-Dimyristoyl-sn-glycero-3-phosphocholine (DMPC), 1-Palmitoyl-2-olcoyl-sn-glycero-3-phosphocholine (POPC), 1,2-dioleoyl-sn-glycero-3-phosphocholine (DOPC), and 1,2-Dioleoyl-sn-glycero-3-phosphoethanolamine (DOPE). In some aspects, the neutral lipid is DSPC.

In some aspects, the molar ratio of the compound to the neutral lipid ranges from about 2:1 to about 8:1.

In some aspects, the steroid is cholesterol. In some aspects, the molar ratio of the compound to cholesterol ranges from about 2:1 to 1:1.

In some aspects, the polymer conjugated lipid is a pegylated lipid. In some aspects, the molar ratio of the compound to the pegylated lipid ranges from about 100:1 to about 25:1. In some aspects, the pegylated lipid is PEG-DAG, a PEG polyethylene (PEG-PE), a PEG-succinoyl-diacylglycerol (PEG-S-DAG), PEG-cer or a PEG dialkyoxypropylcarbamate. In some aspects, the pegylated lipid has the following structure III:

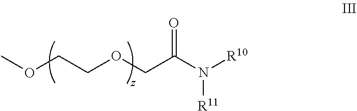

or a pharmaceutically acceptable salt, tautomer or stereoisomer thereof, wherein: R¹⁰ and R¹¹ are each independently a straight or branched, saturated or unsaturated alkyl chain containing from 10 to 30 carbon atoms, wherein the alkyl chain is optionally interrupted by one or more ester bonds; and z has a mean value ranging from 30 to 60. In some aspects, R¹⁰ and R¹¹ are each independently straight, saturated alkyl chains having 12 to 16 carbon atoms. In some aspects, the average z is about 45.

### Start Here

In some aspects, the LNP self-assembles into non-bilayer structures when mixed with polyanionic nucleic acid. In some aspects, the non-bilayer structures have a diameter between 60 nm and 120 nm. In some aspects, the non-bilayer structures have a diameter of about 70 nm, about 80 nm, about 90 nm, or about 100 nm. In some aspects, wherein the nanoparticulate delivery vehicle has a diameter of about 100 nm.

Also provided for herein is a vector or set of vectors comprising any of the nucleotide sequence described herein. Also disclosed herein is a vector comprising an isolated nucleotide sequence disclosed herein.

Also provided for herein is an isolated cell comprising any of the nucleotide sequences or set of isolated nucleotide sequences described herein, optionally wherein the cell is a BHK-21, CHO, HEK293 or variants thereof, 911, HeLa, A549, LP-293, PER.C6, or AE1-2a cell.

Also provided for herein is a kit comprising any of the compositions described herein and instructions for use. Also disclosed herein is a kit comprising a vector or a composition disclosed herein and instructions for use.

Also provided for herein is a method for treating a subject suffering from Covid-19, the method comprising administering to the subject any of the compositions or any of the pharmaceutical compositions described herein.

Also provided for herein is a method for treating a subject infected with or at risk for infection by coronavirus, the method comprising administering to the subject any of the compositions or any of the pharmaceutical compositions described herein.

Also provided for herein is a method for stimulating an immune response in a subject, the method comprising administering to the subject any of the compositions or any of the pharmaceutical compositions described herein.

Also disclosed herein is a method for treating a subject, the method comprising administering to the subject a vector disclosed herein or a pharmaceutical composition disclosed herein.

Also disclosed herein is a method of manufacturing the one or more vectors of any of the above compositions.

Also disclosed herein is a method of manufacturing any of the compositions disclosed herein.

### BRIEF DESCRIPTION OF THE SEVERAL VIEWS OF THE DRAWINGS

These and other features, aspects, and advantages of the present invention will become better understood with regard to the following description, and accompanying drawings, where:

FIG. 1 presents a schematic of the SARS-COV-2 genome structure depicting the at least 14 open reading frames (ORF) identified in. Figure adapted from Zhou et al. (2020) [A pneumonia outbreak associated with a new coronavirus of probable bat origin. Nature, 579(January)].

FIG. 2 depicts the 16 cleavage products of the replicase ORF1ab and related information. Figure adapted from Wu et al. (2020). [A new coronavirus associated with human respiratory disease in China. Nature, 579(January)]

FIG. 3 depicts the general vaccination approach of producing a balanced immune response inducing both neutralizing antibodies (from B cells) as well as effector and memory CD8+ T cell responses for maximum efficacy. SARS-COV-2 genome structure adapted from Zhou et al. (2020) [A pneumonia outbreak associated with a new coronavirus of probable bat origin. Nature, 579(January)].

FIG. 4 demonstrates the known prevalence of the wildtype and D614G variant SARS-Cov-2 Spike protein over time across various geographic locations.

FIG. 5 demonstrates coverage of cassettes encoding only Spike or encoding Spike and the additional predicted concatenated T cell epitopes over the four populations shown. The first column demonstrates the number of SARS-COV-2 epitopes predicted to be presented and the second column demonstrates the expected number of presented epitopes, based on a 0.2 PPV. Each row shows the protection coverage of each population if a certain number of epitopes is used.

FIG. 6A illustrates the number of predicted epitopes presented by each MHC class II allele separately for the Spike protein or the additional predicted concatenated T cell epitopes.

FIG. 6B illustrates the number of the number of SARS-COV-2 epitopes predicted to be presented over the four populations shown from cassettes encoding only Spike (top panel) or encoding Spike and the additional predicted concatenated T cell epitopes (bottom panel).

FIG. 7A presents the number of training samples containing Class I alleles (with at least 10 samples).

FIG. 7B presents a histogram depicting the number of training samples per Class I allele versus the number of alleles.

FIG. 8A shows a Western blot using an anti-Spike S2 antibody for Spike expression in vectors encoding various Spike variations.

FIG. 8B shows a Western blot using an anti-Spike SI antibody for Spike expression in vectors encoding various Spike variations.

FIG. 8C shows a Western blot using an anti-Spike SI antibody for Spike expression in vectors encoding full-length Spike, Spike SI alone, or Spike S2 alone.

FIG. 8D shows a Western blot using an anti-Spike S2 antibody for Spike expression in vectors encoding full-length Spike, Spike SI alone, or Spike S2 alone.

FIG. 9 shows a Western blot using an anti-Spike S2 antibody for Spike expression in vectors encoding various sequence-optimized Spike variations.

FIG. 10A depicts a schematic of PCR-based assay to assess RNA splicing of SARS-CoV-2 transcripts.

FIG. 10B shows PCR amplicons for encoded Spike proteins. Left panel depicts amplicons from cDNA templates from infected 293 cells (“ChAd-Spike (IDT) cDNA”) or from the plasmid encoding the SARS-COV-2 Spike cassette (“Spike Plasmid”). Right panel depicts amplicons from the cDNA of 293 cells infected with a vector encoding Spike SI alone (“SpikeS1”) or full-length Spike (“Spike”).

FIG. 11 shows PCR amplicons for encoded Spike proteins from the cDNA of 293 cells infected with vector encoding various Spike variations.

FIG. 12 presents estimated coverages for the percentage of the indicated ancestry populations having at least one HLA estimated to receive at least one immunogenic epitope encoded by TCE5, where receipt of the immunogenic peptide presentation is considered to occur when an individual's HLA is either (1) known to present an encoded epitope (“validated epitope”), or (2) predicted to present at least 4 (Col. 1), 5 (Col. 2), 6 (Col. 3), or 7 (Col. 4) encoded epitopes (“predicted epitope”; EDGE score >0.01). FA=African American, API=Asian or Pacific Islander, EUR=European, HIS=Hispanic

FIG. 13A presents T cell responses (left panel), Spike-specific IgG antibodies (middle panel) and neutralizing antibodies (right panel) following administration of ChAdV-platforms with Spike-encoding cassettes featuring different sequence optimizations “IDTSpike_{g}” (shown as “Spike V1” or “v1”) or “CTSpike_{g}” (shown as “Spike V2” or “v2”). Balb/c mice immunized with 1×10¹¹ VP ChAdV-based vaccine platform.

FIG. 13B presents T cell responses (left panel), Spike-specific IgG antibodies (middle panel) and neutralizing antibodies (right panel) following administration of SAM-platforms with Spike-encoding cassettes featuring different sequence optimizations “IDTSpike_{g}” (shown as “Spike V1” or “v1”) or “CTSpike_{g}” (shown as “Spike V2” or “v2”). Balb/c mice immunized with 10 μg SAM-based vaccine platform.

FIG. 14 presents Spike-specific IgG antibody production following administration of cither ChAdV-platform (left panel) or SAM-platform (right panel) with unmodified or modified (“CTSpikeF2P_{g}” shown as “SpikeF2P”) Spike-encoding cassettes (all vectors utilize Spike sequence v2). Balb/c mice immunized with 1×10¹¹ VP ChAdV-based vaccine platform or 10 μg SAM-based vaccine platform, as indicated.

FIG. 15A presents T cell responses to Spike (left panel) and T cell responses to the encoded T cell epitopes (right panel) following administration of ChAdV-platforms with a modified Spike-encoding only cassette (“CTSpikeF2P_{g}” shown as “Spike”) and modified Spike together with additional non-Spike T cell epitopes encoded TCE5 (shown as “Spike TCE”). Balb/c mice immunized with 1×10¹¹ VP ChAdV-based vaccine platform. Shown is IFNγ ELISpot, 2 weeks post immunization. T cell response to overlapping peptide pools spanning cither Spike, Nucleocapsid, or Orf3a.

FIG. 15B presents T cell responses to Spike (left panel) and T cell responses to the encoded T cell epitopes (right panel) following administration of SAM-platforms with a modified Spike-encoding only cassette (“CTSpikeF2P_{g}” shown as “Spike”) and modified Spike together with additional non-Spike T cell epitopes encoded TCE5 (shown as “TCE Spike”). Balb/c mice immunized with 10 μg SAM-based vaccine platform. Shown is IFNγ ELISpot, 2 weeks post immunization. T cell response to overlapping peptide pools spanning either Spike, Nucleocapsid, or Orf3a.

FIG. 16A presents T cell responses to Spike (top panel; IFNg ELISpot. Sum of response to 8 overlapping peptide pools spanning Spike antigen), T cell responses to the encoded T cell epitopes (middle panel; IFNg ELISpot. Sum of response to 3 overlapping peptide pools spanning NCap, Membrane, and Orf3a), and Spike-specific IgG antibodies (bottom panel; S1 IgG binding measured by MSD ELISA. Interpolated endpoint titer. Geomean, geometric SD) following immunization with SAM constructs including “IDTSpike_{g}” alone (left columns), IDTSpike_{g} expressed from a first subgenomic promoter followed by TCE5 expressed from a second subgenomic promoter (middle columns), or TCE5 expressed from a first subgenomic promoter followed by IDTSpike_{g} expressed from a second subgenomic promoter (right columns). For T cell responses, Balb/c mice were immunized with 10 μg of each vaccine, n=6/group. Splenocyte isolation at 2-weeks post immunization. For IgG response, Balb/c mice immunized with 10 μg of each vaccine, n=4/group. Serum collected and analyzed at 4-weeks post immunization.

FIG. 16B presents T cell responses to Spike (top panel; IFNg ELISpot. Sum of response to 8 overlapping peptide pools spanning Spike antigen), T cell responses to the encoded T cell epitopes (middle panel; IFNg ELISpot. Sum of response to 3 overlapping peptide pools spanning NCap, Membrane, and Orf3a), and Spike-specific IgG antibodies (bottom panel; S1 IgG binding measured by MSD ELISA. Interpolated endpoint titer. Geomean, geometric SD) following immunization with SAM constructs including “IDTSpike_{g}” alone (first column), IDTSpike_{g} expressed from a first subgenomic promoter followed by TCE6 or TCE7 expressed from a second subgenomic promoter (columns 2 and 4, respectively), or TCE6 or TCE7 expressed from a first subgenomic promoter followed by IDTSpike_{g} expressed from a second subgenomic promoter (columns 3 and 5, respectively). For T cell responses, Balb/c mice were immunized with 10 μg of each vaccine, n=6/group. Splenocyte isolation at 2-weeks post immunization. For IgG response, Balb/c mice immunized with 10 μg of each vaccine, n=4/group. Serum collected and analyzed at 4-weeks post immunization.

FIG. 16C presents T cell responses to Spike (top panel; IFNg ELISpot. Sum of response to 2 overlapping peptide pools spanning Spike antigen), T cell responses to the encoded T cell epitopes (middle panel; IFNg ELISpot. Sum of response to 2 overlapping peptide pools spanning NCap and Orf3a), and Spike-specific IgG antibodies (bottom panel; SI IgG binding measured by MSD ELISA. Interpolated endpoint titer. Geomean, geometric SD) following immunization with SAM constructs including “CTSpike_{g}” alone (first column), CTSpike_{g} expressed from a first subgenomic promoter followed by TCE5 or TCE8 expressed from a second subgenomic promoter (columns 2 and 4, respectively), or TCE5 or TCE8 expressed from a first subgenomic promoter followed by CTSpike_{g} expressed from a second subgenomic promoter (columns 3 and 5, respectively). For T cell responses, Balb/c mice were immunized with 10 μg of each vaccine, n=6/group. Splenocyte isolation at 2-weeks post immunization. For IgG response, Balb/c mice immunized with 10 μg of each vaccine, n=4/group. Scrum collected and analyzed at 4-weeks post immunization.

FIG. 17A presents a map of sequences included in TCE10 for Nucleocapsid, including frames with flanking sequences, validated epitopes, predicted epitopes, mutations, and overlap between frames and mutations.

FIG. 17B presents a map of sequences included in TCE10 for ORF3a, including frames with flanking sequences, validated epitopes, predicted epitopes, mutations, and overlap between frames and mutations.

FIG. 17C presents a map of sequences included in TCE10 for nsp3, including frames with flanking sequences, validated epitopes, predicted epitopes, mutations, and overlap between frames and mutations.

FIG. 17D presents a map of sequences included in TCE10 for Membrane, including frames with flanking sequences, validated epitopes, predicted epitopes, mutations, and overlap between frames and mutations.

FIG. 17E presents a map of sequences included in TCE10 for nsp4, including frames with flanking sequences, validated epitopes, predicted epitopes, mutations, and overlap between frames and mutations.

FIG. 17F presents a map of sequences included in TCE10 for nsp12, including frames with flanking sequences, validated epitopes, predicted epitopes, mutations, and overlap between frames and mutations.

FIG. 18A presents a map of sequences included in TCE9 for nsp12, including frames with flanking sequences, validated epitopes, predicted epitopes, mutations, and overlap between frames and mutations.

FIG. 18B presents a map of sequences included in TCE9 for nsp4, including frames with flanking sequences, validated epitopes, predicted epitopes, mutations, and overlap between frames and mutations.

FIG. 18C presents a map of sequences included in TCE9 for Membrane, including frames with flanking sequences, validated epitopes, predicted epitopes, mutations, and overlap between frames and mutations.

FIG. 18D presents a map of sequences included in TCE9 for nsp3, including frames with flanking sequences, validated epitopes, predicted epitopes, mutations, and overlap between frames and mutations.

FIG. 18E presents a map of sequences included in TCE9 for ORF3a, including frames with flanking sequences, validated epitopes, predicted epitopes, mutations, and overlap between frames and mutations.

FIG. 18F presents a map of sequences included in TCE9 for Nucleocapsid, including frames with flanking sequences, validated epitopes, predicted epitopes, mutations, and overlap between frames and mutations.

FIG. 18G presents a map of sequences included in TCE9 for nsp6, including frames with flanking sequences, validated epitopes, predicted epitopes, mutations, and overlap between frames and mutations.

FIG. 19A presents a map of sequences included in TCE11 for nsp12, including frames with flanking sequences, validated epitopes, predicted epitopes, mutations, and overlap between frames and mutations.

FIG. 19B presents a map of sequences included in TCE11 for Membrane, including frames with flanking sequences, validated epitopes, predicted epitopes, mutations, and overlap between frames and mutations.

FIG. 19C presents a map of sequences included in TCE11 for nsp4, including frames with flanking sequences, validated epitopes, predicted epitopes, mutations, and overlap between frames and mutations.

FIG. 19D presents a map of sequences included in TCE11 for nsp3, including frames with flanking sequences, validated epitopes, predicted epitopes, mutations, and overlap between frames and mutations.

FIG. 20 presents the percentages of shared candidate 9-mer epitope distribution between SARS-COV-2 and SARS-COV (left panel) and between SARS-COV-2 and MERS (right panel).

FIG. 21 presents a schematic outline for selection of RBD sequences for inclusion in a vaccine. BLOSUM62 is a function over (AA, AA)→R which is an expression of homology which is a proxy for functional similarity, where larger means more similar. The average BLOSUM62 similarity over a pair of aligned sequences, a and b is the sum of the BLOSUM62 (aᵢ, bᵢ) divided by the length of a. The average BLOSUM62 distance over two aligned sequences is the negation of the negation of the average BLOSUM62 similarity.

FIG. 22A shows a phylogenetic tree and the viral isolates selected (highlighted) for inclusion of their respective RBD in a vaccine based on an analysis of full-length RBD sequences.

FIG. 22B shows a phylogenetic tree and the viral isolates selected (highlighted) for inclusion of their respective RBD in a vaccine based on an analysis of RBDΔRBM sequences.

FIG. 22C shows a phylogenetic tree and the viral isolates selected (highlighted) for inclusion of their respective RBD in a vaccine based on an analysis of RBM sequences alone.

FIG. 23 shows sarbecovirus clade designations of representative analyzed isolates, including those with the selected RBDs.

FIG. 24 shows RBD amino acid similarity for representative analyzed isolates, including those with the selected RBDs

FIG. 25 shows a schematic of “blended” (top panel) and single-vector (bottom panel) pancorona vaccine strategies.

FIG. 26 shows a Western blot using an anti-SARS-COV-2 polyclonal antibody run under the indicated conditions.

FIG. 27A shows anti-RBD antibody responses to vaccine matched (grey or black bars) and unmatched RBDs (color bars) measured at 4 wk post prime for Group 1 vaccines (MK/KP/KJ).

FIG. 27B shows anti-RBD antibody responses to vaccine matched (grey or black bars) and unmatched RBDs (color bars) measured at 4 wk post prime for Group 2 vaccines (DQ/KJ/MK).

FIG. 27C shows anti-RBD antibody responses to vaccine matched (grey or black bars) and unmatched RBDs (color bars) measured at 4 wk post prime for Group 3 vaccines (GQ/JX/NC).

FIG. 28 shows a pseudovirus neutralization assay (PNA) for at 4 weeks post prime (top panel) and at 4 weeks post boost (bottom panel) for the indicated “blended” samRNA vectors in naïve mice.

FIG. 29 shows a pseudovirus neutralization assay (PNA) for the indicated “blended” samRNA vectors in non-naïve mice.

FIG. 30A shows anti-RBD antibody responses for samRNA vectors expressing a full-length Spike and three RBDs on the same SAM backbone in a homologous prime/boost strategy.

FIG. 30B shows neutralizing antibody titers assessed by PNA for samRNA vectors expressing a full-length Spike and three RBDs on the same SAM backbone in a homologous prime/boost strategy.

FIG. 31 shows neutralizing antibody titers assessed by PNA post boost (bottom panel) and post prime (top panel).

FIG. 32 shows neutralizing antibody titers assessed by PNA for vectors expressing a full-length Spike and three RBDs on the same backbone in a heterologous prime/boost strategy.

FIG. 33 shows relative TCE RNA expression as determined by RT-qPCR and normalized to another non-coronavirus TCE vector.

FIG. 34 shows T-cell responses induced by various TCE-encoding vaccine vectors as measured by ELISpot.

FIG. 35 shows T-cell responses induced by various TCE12 encoding samRNA and ChAdV vaccine vectors as measured by ELISpot.

FIG. 36 illustrates homologous and heterologous prime/boost regimens in Indian rhesus macaques assessing ChAdV and SAM vaccine platforms encoding different isolates of the SARS-COV-2 Spike protein.

FIG. 37A presents T cell responses across multiple Spike T cell epitope pools (top panel; Mean+−SE for each pool), T cell responses for individual NHPs directed to a single large Spike T cell epitope pool over time (middle panel), and Spike-specific IgG antibody titers over time (bottom panel) for Group 1. n=5 NHPs

FIG. 37B presents T cell responses across multiple Spike T cell epitope pools (top panel; Mean+−SE for each pool), T cell responses for individual NHPs directed to a single large Spike T cell epitope pool over time (middle panel), and Spike-specific IgG antibody titers over time (bottom panel) for Group 2. n=5 NHPs

FIG. 37C presents T cell responses across multiple Spike T cell epitope pools (top panel; Mean+−SE for each pool), T cell responses for individual NHPs directed to a single large Spike T cell epitope pool over time (middle panel), and Spike-specific IgG antibody titers over time (bottom panel) for Group 5. n=5 NHPs

FIG. 37D presents T cell responses across multiple Spike T cell epitope pools (top panel; Mean+−SE for each pool), T cell responses for individual NHPs directed to a single large Spike T cell epitope pool over time (middle panel), and Spike-specific IgG antibody titers over time (bottom panel) for Group 6. n=5 NHPs

FIG. 38 presents summaries of T cell responses for individual NHPs directed to a single large Spike T cell epitope pool over time (top panel), T cell responses to the TCE5-encoded epitopes (middle panel), and Spike-specific IgG antibody titers over time (bottom panel) for Group 1. n=5 NHPs

FIG. 39 presents neutralizing antibody production to both the D614G pseudovirus (left panels) and B.1.351 pseudovirus (right panels) following Boost 1 (left columns) and Boost 2 (right columns) for each of the NHP Groups.

FIG. 40 presents neutralizing antibody production comparing the relative Nab titer levels against each of the pseudoviruses following Boost 1 (top panels) and following Boost 2 (bottom panels).

FIG. 41 shows a dosing regimen for Rhesus macaques immunized twice with SAM encoding SARS-COV-2 Spike antigen at specified dose of either 30 μg or 300 μg. Shown are PBMCs assessed by ex-vivo IFNγ ELISpot following overnight stimulation with Spike-specific overlapping peptide pool (left panel) and neutralizing antibodies measured in serum at study week 8 by pseudovirus neutralization assay (right panel).

FIG. 42 shows anti-RBD IgG titers against various RBD domains as assessed by MSD ELISA for single SAM vectors expressing Group 1 (“V1”), Group 2 (“V2”), or Group 3 (“V3”), antigens (SARS-COV2 Spike plus 3 sarbecovirus RBDs) compared to a SAM-SARS-CoV2 vaccine only (“CoV-2 Spike”). Arrows indicate vaccine components. Anti-RBD or Spike data at 4 weeks post vaccination. Each vaccine was administered at a dose of 5 μg in Balb/c mice.

FIG. 43 shows anti-RBD IgG titers against various RBD domains as assessed by MSD ELISA for single SAM vectors expressing Group 2 (“V2”) antigens (SARS-COV2 Spike plus 3 sarbecovirus RBDs) compared to a SAM-SARS-COV2 vaccine only (“CoV-2 Spike”). Arrows indicate vaccine components. Shown is a homologous prime/boost strategy with a boost at 4 weeks post prime. Anti-RBD or Spike data at 4 weeks post boost vaccination (week 8). Each vaccine was administered at a dose of 5 μg in Balb/c mice.

FIG. 44 shows neutralizing antibody titers assessed by PNA for single SAM vectors expressing Group 2 (“V2”) antigens (SARS-COV2 Spike plus 3 sarbecovirus RBDs) compared to a SAM-SARS-COV2 vaccine only (“CoV-2 Spike”). Arrows indicate vaccine components. Shown is a homologous prime/boost strategy with a boost at 4 weeks post prime. Neutralizing titers assessed at 4 weeks post boost vaccination (week 8). Each vaccine was administered at a dose of 5 μg in Balb/c mice.

FIG. 45 shows anti-RBD IgG titers against various RBD domains as assessed by MSD ELISA for single SAM vectors expressing Group 2 (“V2”) antigens (SARS-COV2 Spike plus 3 sarbecovirus RBDs), a combination of a vector expressing Group 2 antigens a vector expressing TCE12 epitope cassette (“BLENDED”), and compared to a SAM-SARS-COV2 vaccine only (“CoV-2 Spike”). Arrows indicate vaccine components. Shown is a homologous prime/boost strategy with a boost at 4 weeks post prime. Anti-RBD or Spike data at 8 weeks post vaccination. Each vaccine was administered at a dose of 5 μg in Balb/c mice, including 5 μg of each separate vector in the blended composition and administered as a single dose.

FIG. 46 shows neutralizing antibody titers assessed by PNA for single SAM vectors expressing Group 2 (“V2”) antigens (SARS-COV2 Spike plus 3 sarbecovirus RBDs), a combination of a vector expressing Group 2 antigens a vector expressing TCE12 epitope cassette (“BLENDED”), and compared to a SAM-SARS-COV2 vaccine only (“CoV-2 Spike”). Arrows indicate vaccine components. Shown is a homologous prime/boost strategy with a boost at 4 weeks post prime. Neutralizing titers assessed at 8 weeks post vaccination. Each vaccine was administered at a dose of 5 μg in Balb/c mice, including 5 μg of each separate vector in the blended composition and administered as a single dose.

FIG. 47 shows T-cell responses induced by TCE12-encoding vaccine vectors against Nsp13, as measured by ELISpot. Shown are responses following of single SAM vectors expressing Group 2 (“3×RBD”) antigens (SARS-COV2 Spike plus 3 sarbecovirus RBDs), a combination of a vector expressing Group 2 antigens and a vector expressing a TCE12 epitope cassette in a blended composition and administered as a single dose (“3×RBD+TCE12”), a combination of a vector expressing Group 2 antigens and a vector expressing a TCE12 epitope cassette administered as separate doses in the right and left leg (“3×RBD+TCE12 (R+L)”), and a vector expressing a TCE12 epitope cassette (“TCE12”), and compared to a SAM-SARS-COV2 vaccine only (“Spike-Delta”). Splenocytes harvested 14 days post vaccination and T-cell responses were measured by ELISpot assay using NSP13 specific overlapping peptide pools. Each vaccine was administered at a dose of 3 μg in Balb/c mice, including 3 μg of each separate vector when in combination.

### DETAILED DESCRIPTION

### I. Definitions

In general, terms used in the claims and the specification are intended to be construed as having the plain meaning understood by a person of ordinary skill in the art. Certain terms are defined below to provide additional clarity. In case of conflict between the plain meaning and the provided definitions, the provided definitions are to be used.

As used herein the term “antigen” is a substance that stimulates an immune response. An antigen can be a neoantigen. An antigen can be a “shared antigen” that is an antigen found among a specific population, e.g., a specific population of SARS-COV-2 patients with or at risk of infection for an infectious disease.

As used herein the term “antigen-based vaccine” is a vaccine composition based on one or more antigens, e.g., a plurality of antigens. The vaccines can be nucleotide-based (e.g., virally based, RNA based, or DNA based), protein-based (e.g., peptide based), or a combination thereof.

As used herein the term “candidate antigen” is a mutation or other aberration giving rise to a sequence that may represent an antigen.

As used herein the term “coding region” is the portion(s) of a gene that encode protein.

As used herein the term “coding mutation” is a mutation occurring in a coding region.

As used herein the term “ORF” means open reading frame.

As used herein the term “missense mutation” is a mutation causing a substitution from one amino acid to another.

As used herein the term “nonsense mutation” is a mutation causing a substitution from an amino acid to a stop codon or causing removal of a canonical start codon.

As used herein the term “frameshift mutation” is a mutation causing a change in the frame of the protein.

As used herein the term “indel” is an insertion or deletion of one or more nucleic acids.

As used herein, the term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Alternatively, sequence similarity or dissimilarity can be established by the combined presence or absence of particular nucleotides, or, for translated sequences, amino acids at selected sequence positions (e.g., sequence motifs).

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information.

As used herein the term “non-stop or read-through” is a mutation causing the removal of the natural stop codon.

As used herein the term “epitope” is the specific portion of an antigen typically bound by an antibody or T cell receptor.

As used herein the term “immunogenic” is the ability to stimulate an immune response, e.g., via T cells, B cells, or both.

As used herein the term “HLA binding affinity” “MHC binding affinity” means affinity of binding between a specific antigen and a specific MHC allele.

As used herein the term “bait” is a nucleic acid probe used to enrich a specific sequence of DNA or RNA from a sample.

As used herein the term “variant” is a difference between a subject's nucleic acids and the reference human genome used as a control.

As used herein the term “variant call” is an algorithmic determination of the presence of a variant, typically from sequencing.

As used herein the term “polymorphism” is a germline variant, i.e., a variant found in all DNA-bearing cells of an individual.

As used herein the term “somatic variant” is a variant arising in non-germline cells of an individual.

As used herein the term “allele” is a version of a gene or a version of a genetic sequence or a version of a protein.

As used herein the term “HLA type” is the complement of HLA gene alleles.

As used herein the term “nonsense-mediated decay” or “NMD” is a degradation of an mRNA by a cell due to a premature stop codon.

As used herein the term “exome” is a subset of the genome that codes for proteins. An exome can be the collective exons of a genome.

As used herein the term “logistic regression” is a regression model for binary data from statistics where the logit of the probability that the dependent variable is equal to one is modeled as a linear function of the dependent variables.

As used herein the term “neural network” is a machine learning model for classification or regression consisting of multiple layers of linear transformations followed by element-wise nonlinearities typically trained via stochastic gradient descent and back -propagation.

As used herein the term “proteome” is the set of all proteins expressed and/or translated by a cell, group of cells, or individual.

As used herein the term “peptidome” is the set of all peptides presented by MHC-I or MHC-II on the cell surface. The peptidome may refer to a property of a cell or a collection of cells (e.g., the infectious disease peptidome, meaning the union of the peptidomes of all cells that are infected by the infectious disease).

As used herein the term “ELISPOT” means Enzyme-linked immunosorbent spot assay—which is a common method for monitoring immune responses in humans and animals.

As used herein the term “dextramers” is a dextran-based peptide-MHC multimers used for antigen-specific T-cell staining in flow cytometry.

As used herein the term “tolerance or immune tolerance” is a state of immune non-responsiveness to one or more antigens, e.g. self-antigens.

As used herein the term “central tolerance” is a tolerance affected in the thymus, either by deleting self-reactive T-cell clones or by promoting self-reactive T-cell clones to differentiate into immunosuppressive regulatory T-cells (Tregs).

As used herein the term “peripheral tolerance” is a tolerance affected in the periphery by downregulating or anergizing self-reactive T-cells that survive central tolerance or promoting these T cells to differentiate into Tregs.

The term “sample” can include a single cell or multiple cells or fragments of cells or an aliquot of body fluid, taken from a subject, by means including venipuncture, excretion, ejaculation, massage, biopsy, needle aspirate, lavage sample, scraping, surgical incision, or intervention or other means known in the art.

The term “subject” encompasses a cell, tissue, or organism, human or non-human, whether in vivo, ex vivo, or in vitro, male or female. The term subject is inclusive of mammals including humans.

The term “mammal” encompasses both humans and non-humans and includes but is not limited to humans, non-human primates, canines, felines, murines, bovines, equines, and porcines.

The term “clinical factor” refers to a measure of a condition of a subject, e.g., disease activity or severity. “Clinical factor” encompasses all markers of a subject's health status, including non-sample markers, and/or other characteristics of a subject, such as, without limitation, age and gender. A clinical factor can be a score, a value, or a set of values that can be obtained from evaluation of a sample (or population of samples) from a subject or a subject under a determined condition. A clinical factor can also be predicted by markers and/or other parameters such as gene expression surrogates. Clinical factors can include infection type (e.g., Coronavirus species), infection sub-type (e.g., SARS-COV-2 variant), and medical history.

The term “antigen-encoding nucleic acid sequences derived from an infection” refers to nucleic acid sequences obtained from infected cells or an infectious disease organism, e.g. via RT-PCR; or sequence data obtained by sequencing the infected cell or infectious disease organism and then synthesizing the nucleic acid sequences using the sequencing data, e.g., via various synthetic or PCR-based methods known in the art. Derived sequences can include nucleic acid sequence variants, such as sequence-optimized nucleic acid sequence variants (e.g., codon-optimized and/or otherwise optimized for expression), that encode the same polypeptide sequence as the corresponding native infectious disease organism nucleic acid sequence. Derived sequences can include nucleic acid sequence variants that encode a modified infectious disease organism polypeptide sequence having one or more (e.g., 1, 2, 3, 4, or 5) mutations relative to a native infectious disease organism polypeptide sequence. For example, a modified polypeptide sequence can have one or more missense mutations relative to the native polypeptide sequence of an infectious disease organism protein.

The term “coronavirus nucleic acid sequence encoding an immunogenic polypeptide” refers to nucleic acid sequences obtained from a coronavirus virus, e.g. via RT-PCR; or sequence data obtained by sequencing a coronavirus virus or a coronavirus virus infected cell, and then synthesizing the nucleic acid sequences using the sequencing data, e.g., via various synthetic or PCR-based methods known in the art. Derived sequences can include nucleic acid sequence variants, such as sequence-optimized nucleic acid sequence variants (e.g., codon-optimized and/or otherwise optimized for expression), that encode the same polypeptide sequence as the corresponding native coronavirus nucleic acid sequence. Derived sequences can include nucleic acid sequence variants that encode a modified coronavirus polypeptide sequence having one or more (e.g., 1, 2, 3, 4, or 5) mutations relative to a native coronavirus polypeptide sequence. For example, a modified Spike polypeptide sequence can have one or more mutations such as one or more missense mutations of R682, R815, K986P, or V987P relative to the native spike polypeptide sequence of a SARS-COV-2 protein.

The term “alphavirus” refers to members of the family Togaviridae, and are positive-sense single-stranded RNA viruses. Alphaviruses are typically classified as either Old World, such as Sindbis, Ross River, Mayaro, Chikungunya, and Semliki Forest viruses, or New World, such as eastern equine encephalitis, Aura, Fort Morgan, or Venezuelan equine encephalitis and its derivative strain TC-83. Alphaviruses are typically self-replicating RNA viruses.

The term “alphavirus backbone” refers to minimal sequence(s) of an alphavirus that allow for self-replication of the viral genome. Minimal sequences can include conserved sequences for nonstructural protein-mediated amplification, a nonstructural protein 1 (nsP1) gene, a nsP2 gene, a nsP3 gene, a nsP4 gene, and a polyA sequence, as well as sequences for expression of subgenomic viral RNA including a subgenomic promoter (e.g., a 26S promoter element).

The term “sequences for nonstructural protein-mediated amplification” includes alphavirus conserved sequence elements (CSE) well known to those in the art. CSEs include, but are not limited to, an alphavirus 5′ UTR, a 51-nt CSE, a 24-nt CSE, a subgenomic promoter sequence (e.g., a 26S subgenomic promoter sequence), a 19-nt CSE, and an alphavirus 3′ UTR.

The term “RNA polymerase” includes polymerases that catalyze the production of RNA polynucleotides from a DNA template. RNA polymerases include, but are not limited to, bacteriophage derived polymerases including T3, T7, and SP6.

The term “lipid” includes hydrophobic and/or amphiphilic molecules. Lipids can be cationic, anionic, or neutral. Lipids can be synthetic or naturally derived, and in some instances biodegradable. Lipids can include cholesterol, phospholipids, lipid conjugates including, but not limited to, polyethyleneglycol (PEG) conjugates (PEGylated lipids), waxes, oils, glycerides, fats, and fat-soluble vitamins. Lipids can also include dilinoleylmethyl-4-dimethylaminobutyrate (MC3) and MC3-like molecules.

The term “lipid nanoparticle” or “LNP” includes vesicle like structures formed using a lipid containing membrane surrounding an aqueous interior, also referred to as liposomes. Lipid nanoparticles includes lipid-based compositions with a solid lipid core stabilized by a surfactant. The core lipids can be fatty acids, acylglycerols, waxes, and mixtures of these surfactants. Biological membrane lipids such as phospholipids, sphingomyelins, bile salts (sodium taurocholate), and sterols (cholesterol) can be utilized as stabilizers. Lipid nanoparticles can be formed using defined ratios of different lipid molecules, including, but not limited to, defined ratios of one or more cationic, anionic, or neutral lipids. Lipid nanoparticles can encapsulate molecules within an outer-membrane shell and subsequently can be contacted with target cells to deliver the encapsulated molecules to the host cell cytosol. Lipid nanoparticles can be modified or functionalized with non-lipid molecules, including on their surface. Lipid nanoparticles can be single-layered (unilamellar) or multi-layered (multilamellar). Lipid nanoparticles can be complexed with nucleic acid. Unilamellar lipid nanoparticles can be complexed with nucleic acid, wherein the nucleic acid is in the aqueous interior. Multilamellar lipid nanoparticles can be complexed with nucleic acid, wherein the nucleic acid is in the aqueous interior, or to form or sandwiched between

Abbreviations: MHC: major histocompatibility complex; HLA: human leukocyte antigen, or the human MHC gene locus; NGS: next-generation sequencing; PPV: positive predictive value; TSNA: tumor-specific neoantigen; FFPE: formalin-fixed, paraffin-embedded; NMD: nonsense-mediated decay; NSCLC: non-small-cell lung cancer; DC: dendritic cell.

It should be noted that, as used in the specification and the appended claims, the singular forms “a,” “an,” and “the” include plural referents unless the context clearly dictates otherwise.

Unless specifically stated or otherwise apparent from context, as used herein the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any terms not directly defined herein shall be understood to have the meanings commonly associated with them as understood within the art of the invention. Certain terms are discussed herein to provide additional guidance to the practitioner in describing the compositions, devices, methods and the like of aspects of the invention, and how to make or use them. It will be appreciated that the same thing may be said in more than one way. Consequently, alternative language and synonyms may be used for any one or more of the terms discussed herein. No significance is to be placed upon whether or not a term is elaborated or discussed herein. Some synonyms or substitutable methods, materials and the like are provided. Recital of one or a few synonyms or equivalents does not exclude use of other synonyms or equivalents, unless it is explicitly stated. Use of examples, including examples of terms, is for illustrative purposes only and does not limit the scope and meaning of the aspects of the invention herein.

All references, issued patents and patent applications cited within the body of the specification are hereby incorporated by reference in their entirety, for all purposes.

### II. Antigen Identification

Research methods for NGS analysis of tumor and normal exome and transcriptomes have been described and applied in the antigen identification space. 6.14.15 Certain optimizations for greater sensitivity and specificity for antigen identification in the clinical setting can be considered. These optimizations can be grouped into two areas, those related to laboratory processes and those related to the NGS data analysis. The research methods described can also be applied to identification of antigens in other settings, such as identification of identifying antigens from an infectious disease organism (e.g., coronavirus), an infection in a subject, or an infected cell of a subject. Examples of optimizations are known to those skilled in the art, for example the methods described in more detail in U.S. Pat. No. 10,055,540, US Application Pub. No. US20200010849A1, international patent application publications WO/2018/195357 and WO/2018/208856, U.S. application Ser. No. 16/606,577, and international patent application PCT/US2020/021508, each herein incorporated by reference, in their entirety, for all purposes.

Methods for identifying antigens (e.g., antigens derived from an infectious disease organism) include identifying antigens that are likely to be presented on a cell surface (e.g., presented by MHC on an infected cell or an immune cell, including professional antigen presenting cells such as dendritic cells), and/or are likely to be immunogenic. As an example, one such method may comprise the steps of: obtaining at least one of exome, transcriptome or whole genome nucleotide sequencing and/or expression data from an infected cell or an infectious disease organism (e.g., coronavirus), wherein the nucleotide sequencing data and/or expression data is used to obtain data representing peptide sequences of each of a set of antigens (e.g., antigens derived from the infectious disease organism); inputting the peptide sequence of each antigen into one or more presentation models to generate a set of numerical likelihoods that each of the antigens is presented by one or more MHC alleles on a cell surface, such as an infected cell of the subject, the set of numerical likelihoods having been identified at least based on received mass spectrometry data; and selecting a subset of the set of antigens based on the set of numerical likelihoods to generate a set of selected antigens.

### IV. Antigens

Antigens can include nucleotides or polypeptides. For example, an antigen can be an RNA sequence that encodes for a polypeptide sequence. Antigens useful in vaccines can therefore include nucleotide sequences or polypeptide sequences.

Disclosed herein are peptides and nucleic acid sequences encoding peptides derived from any polypeptide associated with coronaviruses, including combinations of peptides and nucleic acid sequences encoding peptides derived from any polypeptide associated with distinct coronaviruses. Coronaviruses can include, but are not limited to, a clade 1 Sarbecovirus, a clade 2 Sarbecovirus, or a clade 3 Sarbecovirus. Coronaviruses can include, but are not limited to, Coronaviruses associated with GenBank database accession numbers NC_045512.2, NC_004718.3, MT121216.1, DQ648857.1, GQ153542.1, DQ648856.1, AY278489.2, GQ153540.1, MN996532.2, KJ473811.1, NC_014470.1, KC881005.1, MK211377.1, KJ473816.1, MK211376.1, AY572034.1, KP886809.1, MT072864.1, KF569996.1, JX993987.1, MK211378.1, MK211374.1, KJ473815.1, JX993988.1, DQ071615.1, KT444582.1, MZ206298.1, or KJ473814.1. Coronaviruses can include a betacoronavirus or an alphacoronavirus. Coronaviruses can include a betacoronavirus. Coronaviruses can include an alphacoronavirus. Coronaviruses can include a merbecoviorus. Coronaviruses can include an embecovirus.

Disclosed herein are peptides and nucleic acid sequences encoding peptides derived from any polypeptide associated with coronavirus, a coronavirus infection in a subject, or a coronavirus infected cell of a subject. Antigens can be derived from nucleotide sequences or polypeptide sequences of a coronavirus virus. Polypeptide sequences of coronavirus include, but are not limited to, predicted MHC class I epitopes shown in Table A, predicted MHC class II epitopes shown in Table B, predicted MHC class I epitopes shown in Table C, coronavirus Spike peptides (e.g., peptides derived from SARS-COV-2, such as SEQ ID NO:59), coronavirus Membrane peptides (e.g., peptides derived from SARS-COV-2, such as SEQ ID NO:61), coronavirus Nucleocapsid peptides (e.g., peptides derived from SARS-COV-2, such as SEQ ID NO:62), coronavirus Envelope peptides (e.g., peptides derived from SARS-COV-2, such as SEQ ID NO:63), coronavirus replicase orf1a and orf1b peptides [such as one or more of non-structural proteins (nsp) 1-16], or any other peptide sequence encoded by a coronavirus virus. Peptides and nucleic acid sequences encoding peptides can be derived from the Wuhan-Hu-1 SARS-COV-2 isolate, sometimes referred to as the SARS-COV-2 reference sequence (SEQ ID NO:76; NC_045512.2, herein incorporated by reference for all purposes). Peptides and nucleic acid sequences encoding peptides can be derived from an isolate distinct from the Wuhan-Hu-1 SARS-COV-2 isolate, such as isolates having one or more mutations in proteins (also referred to as protein variants) with reference to the Wuhan-Hu-1 isolate. Vaccination strategies can include multiple vaccines with peptides and nucleic acid sequences encoding peptides derived from distinct isolates. For example, as an illustrative non-limiting example, a vaccine encoding a Spike protein from the Wuhan-Hu-1 SARS-COV-2 isolate can be administered, followed by subsequent administration of a vaccine encoding a Spike protein from the B.1.351 (“South African”) SARS-COV-2 isolate (e.g., SEQ ID NO: 112) or from the B.1.1.7 (“UK”) SARS-COV-2 isolate (e.g., SEQ ID NO:110). The one or more variants can include, but are not limited to, mutations in the coronavirus Spike protein, coronavirus Membrane protein, coronavirus Nucleocapsid protein, coronavirus Envelope protein, coronavirus replicase orf1a and orf1b protein [such as one or more of non-structural proteins (nsp) 1-16], or any other protein sequences encoded by a coronavirus. Variants can be selected based on prevalence of the mutation among coronavirus subtypes/isolates, such as mutations/variants that are present in 1% or greater, 2% or greater, 3% or greater, 4% or greater, 5% or greater, 6% or greater, 7% or greater, 8% or greater, 9% or greater, 10% or greater, 20% or greater, 30% or greater, 40% or greater, 50% or greater, 60% or greater, 70% or greater, 80% or greater, 90% or greater of coronavirus subtypes/isolates. Examples of mutations in greater than 1% of isolates are shown in Table 1. Variants can be selected based on prevalence of the mutation among coronavirus subtypes/isolates present in a specific population, such as a specific demographic or geographic population. An illustrative non-limiting example of a prevalent variant/mutation is the Spike D614G missense mutation found in 60.05% of genomes sequenced worldwide, and 70.46% and 58.49% of the sequences in Europe and North America, respectively. Accordingly, vaccines can be designed to encode at least one immunogenic polypeptide corresponding to a polypeptide encoded by a coronavirus subtype the subject is infected with or at risk for infection by, such as for use in prophylactic vaccines for a specific demographic or geographic population at risk for infection by the specific coronavirus subtype/isolate. Vaccines can be designed to encode at least one immunogenic polypeptide corresponding to a polypeptide encoded by coronavirus and at least one immunogenic polypeptide corresponding to a polypeptide encoded by a Coronavirus species and/or sub-species other than SARS-COV-2, e.g., the Severe acute respiratory syndrome (SARS) 2002-associated species (NC_004718.3, herein incorporated by reference for all purposes) and/or Middle East respiratory syndrome (MERS) 2012-associated species (NC_019843.3, herein incorporated by reference for all purposes). Vaccines can be designed to encode at least one immunogenic polypeptide corresponding to a polypeptide encoded by SARS-COV-2 that is conserved (e.g., 100% amino acid sequence conservation between epitopes) between SARS-COV-2 and a Coronavirus species and/or sub-species other than SARS-CoV-2, e.g., Severe acute respiratory syndrome (SARS) and/or Middle East respiratory syndrome (MERS) species. SARS-COV-2 epitopes that are conserved between SARS-COV-2 and a Coronavirus species and/or sub-species other than SARS-COV-2 can include epitopes derived from a Coronavirus Spike protein, a Coronavirus Membrane protein, a Coronavirus Nucleocapsid protein, a Coronavirus Envelope protein, a Coronavirus replicase orf1a and orf1b protein [such as one or more of non-structural proteins (nsp) 1-16], or any other protein sequences encoded by a Coronavirus.

Antigens can be selected as part of a “pancorona” vaccine platform that provide broad immunogenicity against multiple coronaviruses, such as multiple coronaviruses in the Sarbecovirus subgenus, which includes the human pathogens SARS-COV and SARS-COV-2. Pancorona vaccines can include encoding receptor binding domains (RBDs) derived from multiple coronaviruses. Pancorona vaccines can include at least two distinct coronavirus receptor binding domain (RBD) derived nucleic acid sequences encoding at least two distinct RBD domain.

Selection of distinct RBD domains for inclusion in a pancoronavirus vaccine can include analysis of RBD domain sequence similarity to maximize coverage of diverse coronaviruses (e.g., coverage of multiple Sarbecovirus clades). Analysis of sequence similarity can be based upon different sub-domains of an RBD. Without wishing to be bound by theory, selecting RBDs based on the RBM alone can result in selection based on the most varied domain, while choosing based on the RBD sequence outside of the RBM (RBDΔRBM) can result in a selection based on the conservative domain. Both selection criteria have potential advantages. More conserved regions may be better immunogens for antibodies that recognize multiple coronaviruses. Selection based on RBM may provide greater coverage to diverse RBMs. The whole RBD group can combine the advantages of both, but is potentially biased to the conserved domains.

Pancorona vaccines can include at least two distinct RBD domains are collectively at least 70% identical by amino acid composition to RBD domains from at least two of a clade 1 Sarbecovirus, a clade 2 Sarbecovirus, or a clade 3 Sarbecovirus. Pancorona vaccines can include

Pancorona vaccines can include at least two distinct RBD domains are collectively at least 70%, 75%, 80%, 85%, or 90% identical by amino acid composition to RBD domains from each of: (A) the clade 3 Sarbecovirus; and (B) the clade 1 Sarbecovirus and/or the clade 2 Sarbecovirus. Pancorona vaccines can include at least two distinct RBD domains that are collectively at least 70%, 75%, 80%, 85%, or 90% identical by amino acid composition to RBD domains from each of NC_045512.2, NC_004718.3, MT121216.1, DQ648857.1, GQ153542.1, DQ648856.1, AY278489.2, GQ153540.1, MN996532.2, KJ473811.1, NC_014470.1, KC881005.1, MK211377.1, KJ473816.1, MK211376.1, AY572034.1, KP886809.1, MT072864.1, KF569996.1, JX993987.1, MK211378.1, MK211374.1, KJ473815.1, JX993988.1, DQ071615.1, KT444582.1, MZ206298.1, and KJ473814.1.

Pancorona vaccines can include selecting RBD domains based on an analysis of sequence similarity between only respective RBM domains, such as including at least two distinct RBD domains that include RBM domains that are collectively at least 30%, 35%, 40%, 45%, 50%, 55%, 60%, 65%, 70%, 75%, 80%, 85%, or 90% identical by amino acid composition to RBM domains from at least two of a clade 1 Sarbecovirus, a clade 2 Sarbecovirus, or a clade 3 Sarbecovirus.

Pancorona vaccines can include selecting RBD domains based on an analysis of sequence similarity between the RBD sequence without the RBM domain sequence (RBDΔRBM), such as at least two distinct RBDs where amino acid sequences of the distinct RBD domains other than the amino acid sequences of their respective RBM domains including at least two distinct RBD domains includes RBM domains that are collectively at least 70%, 75%, 80%, 85%, or 90% identical by amino acid composition to RBM domains from at least two of a clade 1 Sarbecovirus, a clade 2 Sarbecovirus, or a clade 3 Sarbecovirus.

Pancorona vaccines can include at least three distinct coronavirus RBD derived nucleic acid sequences encoding at least three distinct RBD domain. Pancorona vaccines can include at least three distinct coronavirus RBD derived nucleic acid sequences encoding at least three distinct RBD domain.

Pancorona vaccines can include at least four distinct RBD domains are collectively at least 70% identical by amino acid composition to RBD domains from each of the clade 1 Sarbecovirus, the clade 2 Sarbecovirus, and the clade 3 Sarbecovirus.

Pancorona vaccines can include at least two, at least three, or at least four distinct coronavirus RBD derived nucleic acid sequences including at least one from a betacoronavirus RBD derived nucleic acid sequences. Pancorona vaccines can include at least two, at least three, or at least four distinct coronavirus RBD derived nucleic acid sequences including each from a betacoronavirus RBD derived nucleic acid sequences. Pancorona vaccines can include at least two, at least three, or at least four distinct coronavirus RBD derived nucleic acid sequences including at least one from a betacoronavirus RBD derived nucleic acid sequence, an alphacoronavirus RBD derived nucleic acid sequence, and combinations thereof.

Pancorona vaccines can include at least two, at least three, or at least four distinct coronavirus RBD derived nucleic acid sequences including at least one from a sarbecovirus RBD derived nucleic acid sequence, a merbecoviorus RBD derived nucleic acid sequence, a embecovirus RBD derived nucleic acid sequence, and combinations thereof.

Pancorona vaccines can include at least two distinct coronavirus RBD derived nucleic acid sequences that are encoded by a single polynucleotide sequence. Pancorona vaccines can include at least two distinct coronavirus RBD derived nucleic acid sequences that are encoded by a single antigen cassette (e.g., a multi-cistronic cassette). Pancorona vaccines can include at least two distinct coronavirus RBD derived nucleic acid sequences that are encoded by separate polynucleotide sequences (e.g., where each RBD is encoded on a separate viral backbone).

RBD domains can include a trimerization domain. Distinct RBD domains can include a distinct trimerization domain. Distinct RBD domains can include the same trimerization domain. RBD trimerization domains include, but are not limited to, a T4 trimerization domain, a MTQ trimerization domain, and a GCN4 trimerization domain. RBD domains can include a coronavirus derived signal-peptide, such as a SARS-COV-2 derived signal-peptide. RBD domains can include an influenza Hemagglutinin, tissue plasminogen activator, and/or Ag2/PRA derived signal-peptide. RBD derived nucleic acid sequences can be linked together with a peptide-linker encoding nucleic acid sequence. Peptide-linker encoding nucleic acid sequences can include a 2A ribosome skipping sequence element (e.g., a E2A ribosome skipping sequence element, a P2A ribosome skipping sequence element, a F2A ribosome skipping sequence element, or a T2A sequence ribosome skipping sequence element). Peptide-linker encoding nucleic acid sequences can include a cleavable peptide linker (e.g., a TEV cleavage site or a furin cleavage site).

Encoded RBD domains can encode only the RBD domain or can be encoded by a Spike protein encoding the RBD domain (e.g., full-length SARS-COV-2 Spike).

Antigens can be selected that are predicted to be presented on the cell surface of a cell, such as an infected cell or an immune cell, including professional antigen presenting cells such as dendritic cells. Antigens can be selected that are predicted to be immunogenic. Exemplary antigens predicted using the methods described herein to be presented on the cell surface by an MHC include predicted MHC class I epitopes shown in Table A, predicted MHC class II epitopes shown in Table B, and predicted MHC class I epitopes shown in Table C.

Antigens can be selected that have been validated to be presented by a specific HLA and/or stimulate an immune response, such as previously reported/validated in the literature (for example, as in Nelde et al. [Nature Immunology volume 22, pages 74-85 2021], Tarke et al. 2021, or Schelien et al. [bioRxiv 2020.08.13.249433]). The magnitude of stimulation of an immune response can be used to guide epitope/antigen selection, such as to select epitopes that stimulate as robust an immune response as possible, including when cassettes have a size constraint. As an illustrative non-limiting example of magnitude based-selection, the following can be used (1) An individual's magnitude is the sum of all epitope magnitudes across their respective diplotype alleles; (2) Each epitopes magnitude=(magnitude of response)×(Frequency of positive response/100), [e.g., using values found in Tarke et al. (Comprehensive analysis of T cell immunodominance and immunoprevalence of SARS-COV-2 epitopes in COVID-19 cases. Cell Rep Med. 2021 Feb. 16; 2(2):100204. doi: 10.1016/j.xcrm.2021.100204. Epub 2021 Jan. 26.), herein incorporated by reference for all purposes]; (3) exclusion of epitopes other than those from starting proteins that span mutations with >5% frequency, optionally with mutations allowed in flanking regions; and/or (4) cassette order determined to minimize unintended junction epitopes across adjacent frames, as well as minimize consecutive frames in the same protein to reduce chance of functional protein fragments, as described herein.

A cassette can be constructed to encode one or more validated epitopes and/or at least 4, 5, 6, or 7 predicted epitopes, wherein at least 85%, 90%, or 95% of a population carries at least one HLA validated to present at least one of the one or more validated epitopes and/or at least one HLA predicted to present each of the at least 4, 5, 6, or 7 predicted epitopes. A cassette can be constructed to encode one or more validated epitopes and at least 4, 5, 6, or 7 predicted epitopes, wherein at least 85%, 90%, or 95% of a population carries at least one HLA validated to present at least one of the one or more validated epitopes or at least one HLA predicted to present each of the at least 4, 5, 6, or 7 predicted epitopes.

One or more polypeptides encoded by an antigen nucleotide sequence can comprise at least one of: a binding affinity with MHC with an IC50 value of less than 1000 nM, for MHC Class I peptides a length of 8-15, 8, 9, 10, 11, 12, 13, 14, or 15 amino acids, presence of sequence motifs within or near the peptide promoting proteasome cleavage, and presence or sequence motifs promoting TAP transport. For MHC Class II peptides a length 6-30, 6, 7, 8, 9, 10, 11, 12, 13, 14, 15, 16, 17, 18, 19, 20, 21, 22, 23, 24, 25, 26, 27, 28, 29, or 30 amino acids, presence of sequence motifs within or near the peptide promoting cleavage by extracellular or lysosomal proteases (e.g., cathepsins) or HLA-DM catalyzed HLA binding.

One or more antigens can be presented on the surface of an infected cell (e.g., a coronavirus infected cell).

One or more antigens can be immunogenic in a subject having or suspected to have an infection (e.g., a coronavirus infection), e.g., capable of stimulating a T cell response and/or a B cell response in the subject. One or more antigens can be immunogenic in a subject at risk of an infection (e.g., a coronavirus infection), e.g., capable of stimulating a T cell response and/or a B cell response in the subject that provides immunological protection (i.e., immunity) against the infection, e.g., such as stimulating the production of memory T cells, memory B cells, or antibodies specific to the infection.

One or more antigens can be capable of stimulating a B cell response, such as the production of antibodies that recognize the one or more antigens (e.g., antibodies that recognize a coronavirus antigen and/or virus). Antibodies can recognize linear polypeptide sequences or recognize secondary and tertiary structures. Accordingly, B cell antigens can include linear polypeptide sequences or polypeptides having secondary and tertiary structures, including, but not limited to, full-length proteins, protein subunits, protein domains, or any polypeptide sequence known or predicted to have secondary and tertiary structures. Antigens capable of stimulating a B cell response to an infection can be antigens found on the surface of an infectious disease organism (e.g., coronavirus). Antigens capable of stimulating a B cell response to an infection can be an intracellular antigen expressed in an infectious disease organism. coronavirus antigens capable of stimulating a B cell response include, but are not limited to, coronavirus Spike peptides, coronavirus Membrane peptides, coronavirus Nucleocapsid peptides, and coronavirus Envelope peptides.

One or more antigens can include a combination of antigens capable of stimulating a T cell response (e.g., peptides including predicted T cell epitope sequences) and distinct antigens capable of stimulating a B cell response (e.g., full-length proteins, protein subunits, protein domains).

One or more antigens that stimulate an autoimmune response in a subject can be excluded from consideration in the context of vaccine generation for a subject.

The size of at least one antigenic peptide molecule (e.g., an epitope sequence) can comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120 or greater amino molecule residues, and any range derivable therein. In specific embodiments the antigenic peptide molecules are equal to or less than 50 amino acids.

Antigenic peptides and polypeptides can be: for MHC Class I 15 residues or less in length and usually consist of between about 8 and about 11 residues, particularly 9 or 10 residues; for MHC Class II, 6-30 residues, inclusive.

If desirable, a longer peptide can be designed in several ways. In one case, when presentation likelihoods of peptides on HLA alleles are predicted or known, a longer peptide could consist of either: (1) individual presented peptides with an extensions of 2-5 amino acids toward the N- and C-terminus of each corresponding gene product; (2) a concatenation of some or all of the presented peptides with extended sequences for each. In another case, when sequencing reveals a long (>10 residues) epitope sequence present, a longer peptide would consist of: (3) the entire stretch of novel infectious disease-specific amino acids—thus bypassing the need for computational or in vitro test-based selection of the strongest HLA-presented shorter peptide. In both cases, use of a longer peptide allows endogenous processing by patient cells and may lead to more effective antigen presentation leading to increased T cell responses. Longer peptides can also include a full-length protein, a protein subunit, a protein domain, and combinations thereof of a peptide, such as those expressed in an infectious disease organism. Longer peptides (e.g., full-length protein, protein subunit, or protein domain) and combinations thereof can be included to stimulate a B cell response.

Antigenic peptides and polypeptides can be presented on an HLA protein. In some aspects antigenic peptides and polypeptides are presented on an HLA protein with greater affinity than a wild-type peptide. In some aspects, an antigenic peptide or polypeptide can have an IC50 of at least less than 5000 nM, at least less than 1000 nM, at least less than 500 nM, at least less than 250 nM, at least less than 200 nM, at least less than 150 nM, at least less than 100 nM, at least less than 50 nM or less.

In some aspects, antigenic peptides and polypeptides do not stimulate an autoimmune response and/or invoke immunological tolerance when administered to a subject.

Also provided are compositions comprising at least two or more antigenic peptides. In some embodiments the composition contains at least two distinct peptides. At least two distinct peptides can be derived from the same polypeptide. By distinct polypeptides is meant that the peptide vary by length, amino acid sequence, or both. The peptides can be derived from any polypeptide known to or suspected to be associated with an infectious disease organism, or peptides derived from any polypeptide known to or have been found to have altered expression in an infected cell in comparison to a normal cell or tissue (e.g., an infectious disease polynucleotide or polypeptide, including infectious disease polynucleotides or polypeptides with expression restricted to a host cell).

Antigenic peptides and polypeptides having a desired activity or property can be modified to provide certain desired attributes, e.g., improved pharmacological characteristics, while increasing or at least retaining substantially all of the biological activity of the unmodified peptide to bind the desired MHC molecule and activate the appropriate T cell. For instance, antigenic peptide and polypeptides can be subject to various changes, such as substitutions, either conservative or non-conservative, where such changes might provide for certain advantages in their use, such as improved MHC binding, stability or presentation. By conservative substitutions is meant replacing an amino acid residue with another which is biologically and/or chemically similar, e.g., one hydrophobic residue for another, or one polar residue for another. The substitutions include combinations such as Gly, Ala; Val, Ile, Leu, Met; Asp, Glu; Asn, Gln; Ser, Thr; Lys, Arg; and Phe, Tyr. The effect of single amino acid substitutions may also be probed using D-amino acids. Such modifications can be made using well known peptide synthesis procedures, as described in e.g., Merrifield, Science 232:341-347 (1986), Barany & Merrifield, The Peptides, Gross & Meienhofer, cds. (N.Y., Academic Press), pp. 1-284 (1979); and Stewart & Young, Solid Phase Peptide Synthesis, (Rockford, Ill., Pierce), 2d Ed. (1984).

Modifications of peptides and polypeptides with various amino acid mimetics or unnatural amino acids can be particularly useful in increasing the stability of the peptide and polypeptide in vivo. Stability can be assayed in a number of ways. For instance, peptidases and various biological media, such as human plasma and serum, have been used to test stability. See, e.g., Verhoef et al., Eur. J. Drug Metab Pharmacokin. 11:291-302 (1986). Half-life of the peptides can be conveniently determined using a 25% human serum (v/v) assay. The protocol is generally as follows. Pooled human serum (Type AB, non-heat inactivated) is delipidated by centrifugation before use. The serum is then diluted to 25% with RPMI tissue culture media and used to test peptide stability. At predetermined time intervals a small amount of reaction solution is removed and added to either 6% aqueous trichloracetic acid or ethanol. The cloudy reaction sample is cooled (4 degrees C.) for 15 minutes and then spun to pellet the precipitated serum proteins. The presence of the peptides is then determined by reversed-phase HPLC using stability-specific chromatography conditions.

The peptides and polypeptides can be modified to provide desired attributes other than improved serum half-life. For instance, the ability of the peptides to stimulate CTL activity can be enhanced by linkage to a sequence which contains at least one epitope that is capable of inducing a T helper cell response. Immunogenic peptides/T helper conjugates can be linked by a spacer molecule. The spacer is typically comprised of relatively small, neutral molecules, such as amino acids or amino acid mimetics, which are substantially uncharged under physiological conditions. The spacers are typically selected from, e.g., Ala, Gly, or other neutral spacers of nonpolar amino acids or neutral polar amino acids. It will be understood that the optionally present spacer need not be comprised of the same residues and thus can be a hetero- or homo-oligomer. When present, the spacer will usually be at least one or two residues, more usually three to six residues. Alternatively, the peptide can be linked to the T helper peptide without a spacer.

Polypeptides encoding antigens can be modified to alter processing of the polypeptides, such as protease cleavage and/or other post-translation processing. Polypeptides encoding antigens can be modified such that the antigen favors a specific conformation. Polypeptides encoding antigens can be modified such that the mutations (e.g., one or more missense mutations) disrupt a specific conformation in the antigen, such as through the introduction of prolines that disrupt secondary and tertiary structures (e.g., alpha-helix or beta-sheet formation). Altering, reducing, or eliminating processing or conformation changes may, in some instances, bias the antigen to favor states favorable to neutralizing antibody production. In a series of illustrative examples, SARS-COV-2 Spike mutations at amino acids 682, 815, 987, and 988 are engineered to bias the Spike protein to remain in a predominantly prefusion state, a potentially preferable state for antibody-mediated neutralization. Specifically, without wishing to be bound by theory, mutations at R682 (e.g., R682V) disrupt the Furin cleavage site involved in processing Spike into S1 and S2; mutations at R815 (e.g., R815N) disrupt the cleavage site within S2; and mutations at K986 and V987, such as K986P and V987P introducing two prolines, that interfere with the secondary structure of Spike making it less likely to be processed from the pre to post fusion state. Accordingly, an antigen cassette can encode a modified Spike protein having at least one mutation selected from: a Spike R682V mutation, a Spike R815N mutation, a Spike K986P mutation, a Spike V987P mutation, and combinations thereof with reference the Wuhan-Hu-1 isolate (see SEQ ID NO:59 reference and SEQ ID NO:60/SEQ ID NO:90 containing mutations). Modified polypeptide sequences can be at least 60%, 70%, 80%, or 90% identical to a native coronavirus polypeptide sequence. Modified polypeptide sequences can be at least 91%, 92%, 93%, or 94% identical to a native coronavirus polypeptide sequence. Modified polypeptide sequences can be at least 95%, 96%, 97%, 98%, or 99% identical to a native coronavirus polypeptide sequence. Modified polypeptide sequences can be at least 99.1%, 99.2%, 99.3%, 99.4%, 99.5%, 99.6%, 99.7%, 99.8%, or 99.9% identical to a native coronavirus polypeptide sequence.

An antigenic peptide can be linked to the T helper peptide either directly or via a spacer either at the amino or carboxy terminus of the peptide. The amino terminus of either the antigenic peptide or the T helper peptide can be acylated. Exemplary T helper peptides include tetanus toxoid 830-843, influenza 307-319, malaria circumsporozoite 382-398 and 378-389.

Proteins or peptides can be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and can be found at computerized databases known to those of ordinary skill in the art. One such database is the National Center for Biotechnology Information's Genbank and GenPept databases located at the National Institutes of Health website. The coding regions for known genes can be amplified and/or expressed using the techniques disclosed herein or as would be known to those of ordinary skill in the art. Alternatively, various commercial preparations of proteins, polypeptides and peptides are known to those of skill in the art.

In a further aspect, an antigen includes a nucleic acid (e.g. polynucleotide) that encodes an antigenic peptide or portion thereof. The polynucleotide can be, e.g., DNA, cDNA, PNA, CNA, RNA (e.g., mRNA), either single- and/or double-stranded, or native or stabilized forms of polynucleotides, such as, e.g., polynucleotides with a phosphorothioate backbone, or combinations thereof and it may or may not contain introns. A polynucleotide sequence encoding an antigen can be sequence-optimized to improve expression, such as through improving transcription, translation, post-transcriptional processing, and/or RNA stability. For example, polynucleotide sequence encoding an antigen can be codon-optimized. “Codon-optimization” herein refers to replacing infrequently used codons, with respect to codon bias of a given organism, with frequently used synonymous codons. Polynucleotide sequences can be optimized to improve post-transcriptional processing, for example optimized to reduce unintended splicing, such as through removal of splicing motifs (e.g., canonical and/or cryptic/non-canonical splice donor, branch, and/or acceptor sequences) and/or introduction of exogenous splicing motifs (e.g., splice donor, branch, and/or acceptor sequences) to bias favored splicing events. Exogenous intron sequences include, but are not limited to, those derived from SV40 (e.g., an SV40 mini-intron [SEQ ID NO:88]) and derived from immunoglobulins (e.g., human β-globin gene). Exogenous intron sequences can be incorporated between a promoter/enhancer sequence and the antigen(s) sequence. Exogenous intron sequences for use in expression vectors are described in more detail in Callendret et al. (Virology. 2007 Jul. 5; 363(2): 288-302), herein incorporated by reference for all purposes. Polynucleotide sequences can be optimized to improve transcript stability, for example through removal of RNA instability motifs (e.g., AU-rich elements and 3′ UTR motifs) and/or repetitive nucleotide sequences. Polynucleotide sequences can be optimized to improve accurate transcription, for example through removal of cryptic transcriptional initiators and/or terminators. Polynucleotide sequences can be optimized to improve translation and translational accuracy, for example through removal of cryptic AUG start codons, premature polyA sequences, and/or secondary structure motifs. Polynucleotide sequences can be optimized to improve nuclear export of transcripts, such as through addition of a Constitutive Transport Element (CTE), RNA Transport Element (RTE), or Woodchuck Posttranscriptional Regulatory Element (WPRE). Nuclear export signals for use in expression vectors are described in more detail in Callendret et al. (Virology. 2007 Jul. 5; 363(2): 288-302), herein incorporated by reference for all purposes. Polynucleotide sequences can be optimized with respect to GC content, for example to reflect the average GC content of a given organism. Sequence optimization can balance one or more sequence properties, such as transcription, translation, post-transcriptional processing, and/or RNA stability. Sequence optimization can generate an optimal sequence balancing each of transcription, translation, post-transcriptional processing, and RNA stability. Sequence optimization algorithms are known to those of skill in the art, such as GeneArt (Thermo Fisher), Codon Optimization Tool (IDT), Cool Tool, SGI-DNA (La Jolla California). One or more regions of an antigen-encoding protein can be sequence-optimized separately. As a non-limiting illustrative example, coronavirus Spike protein can be sequence-optimized (or unoptimized) in the SI region of the protein and the S2 region is separately optimized (e.g., optimized using a different algorithm and/or optimized for one or more sequence properties specific for the S2 region).

A method disclosed herein can also include identifying one or more T cells that are antigen-specific for at least one of the antigens in the subset. In some embodiments, the identification comprises co-culturing the one or more T cells with one or more of the antigens in the subset under conditions that expand the one or more antigen-specific T cells. In further embodiments, the identification comprises contacting the one or more T cells with a tetramer comprising one or more of the antigens in the subset under conditions that allow binding between the T cell and the tetramer. In even further embodiments, the method disclosed herein can also include identifying one or more T cell receptors (TCR) of the one or more identified T cells. In certain embodiments, identifying the one or more T cell receptors comprises sequencing the T cell receptor sequences of the one or more identified T cells. The method disclosed herein can further comprise genetically engineering a plurality of T cells to express at least one of the one or more identified T cell receptors; culturing the plurality of T cells under conditions that expand the plurality of T cells; and infusing the expanded T cells into the subject. In some embodiments, genetically engineering the plurality of T cells to express at least one of the one or more identified T cell receptors comprises cloning the T cell receptor sequences of the one or more identified T cells into an expression vector; and transfecting each of the plurality of T cells with the expression vector. In some embodiments, the method disclosed herein further comprises culturing the one or more identified T cells under conditions that expand the one or more identified T cells; and infusing the expanded T cells into the subject.

Also disclosed herein is an isolated T cell that is antigen-specific for at least one selected antigen in the subset.

A still further aspect provides an expression vector capable of expressing a polypeptide or portion thereof. Expression vectors for different cell types are well known in the art and can be selected without undue experimentation. Generally, DNA is inserted into an expression vector, such as a plasmid, in proper orientation and correct reading frame for expression. If necessary, DNA can be linked to the appropriate transcriptional and translational regulatory control nucleotide sequences recognized by the desired host, although such controls are generally available in the expression vector. The vector is then introduced into the host through standard techniques. Guidance can be found e.g. in Sambrook et al. (1989) Molecular Cloning, A Laboratory Manual, Cold Spring Harbor Laboratory, Cold Spring Harbor, N.Y.

### V. Vaccine Compositions

Also disclosed herein is an immunogenic composition, e.g., a vaccine composition, capable of raising a specific immune response, e.g., an infectious disease organism-specific immune response. Vaccine compositions typically comprise one or a plurality of antigens, e.g., selected using a method described herein. Vaccine compositions can also be referred to as vaccines.

A vaccine can contain between 1 and 30 peptides, 2, 3, 4, 5, 6, 7, 8, 9, 10, 11, 12, 13, 14, 15, 16, 17, 18, 19, 20, 21, 22, 23, 24, 25, 26, 27, 28, 29, or 30 different peptides, 6, 7, 8, 9, 10 11, 12, 13, or 14 different peptides, or 12, 13 or 14 different peptides. Peptides can include post-translational modifications. A vaccine can contain between 1 and 100 or more nucleotide sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different nucleotide sequences, 6, 7, 8, 9, 10 11, 12, 13, or 14 different nucleotide sequences, or 12, 13 or 14 different nucleotide sequences. A vaccine can contain between 1 and 30 antigen sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different antigen sequences, 6, 7, 8, 9, 10 11, 12, 13, or 14 different antigen sequences, or 12, 13 or 14 different antigen sequences.

A vaccine can contain between 1 and 30 antigen-encoding nucleic acid sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more different antigen-encoding nucleic acid sequences, 6, 7, 8, 9, 10 11, 12, 13, or 14 different antigen-encoding nucleic acid sequences, or 12, 13 or 14 different antigen-encoding nucleic acid sequences. Antigen-encoding nucleic acid sequences can refer to the antigen encoding portion of an “antigen cassette.” Features of an antigen cassette are described in greater detail herein. An antigen-encoding nucleic acid sequence can contain one or more epitope-encoding nucleic acid sequences (e.g., an antigen-encoding nucleic acid sequence encoding concatenated T cell epitopes).

A vaccine can contain between 1 and 30 distinct epitope-encoding nucleic acid sequences, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more distinct epitope-encoding nucleic acid sequences, 6, 7, 8, 9, 10 11, 12, 13, or 14 distinct epitope-encoding nucleic acid sequences, or 12, 13 or 14 distinct epitope-encoding nucleic acid sequences. Epitope-encoding nucleic acid sequences can refer to sequences for individual epitope sequences, such as each of the T cell epitopes in an antigen-encoding nucleic acid sequence encoding concatenated T cell epitopes.

A vaccine can contain at least two repeats of an epitope-encoding nucleic acid sequence. A used herein, a “repeat” refers to two or more iterations of an identical nucleic acid epitope-encoding nucleic acid sequence (inclusive of the optional 5′ linker sequence and/or the optional 3′ linker sequences described herein) within an antigen-encoding nucleic acid sequence. In one example, the antigen-encoding nucleic acid sequence portion of a cassette encodes at least two repeats of an epitope-encoding nucleic acid sequence. In further non-limiting examples, the antigen-encoding nucleic acid sequence portion of a cassette encodes more than one distinct epitope, and at least one of the distinct epitopes is encoded by at least two repeats of the nucleic acid sequence encoding the distinct epitope (i.e., at least two distinct epitope-encoding nucleic acid sequences). In illustrative non-limiting examples, an antigen-encoding nucleic acid sequence encodes epitopes A, B, and C encoded by epitope-encoding nucleic acid sequences epitope-encoding sequence A (E_{A}), epitope-encoding sequence B (E_{B}), and epitope-encoding sequence C (E_{C}), and examplary antigen-encoding nucleic acid sequences having repeats of at least one of the distinct epitopes are illustrated by, but is not limited to, the formulas below:
Repeat of one distinct epitope (repeat of epitope A):

E_{A}-E_{B}-E_{C}-E_{A}; or

E_{A}-E_{A}-E_{E}_{B}-E_{C}
Repeat of multiple distinct epitopes (repeats of epitopes A, B, and C):

E_{A}-E_{B}-E_{C}-E_{A}-E_{B}-E_{C}; or

E_{A}-E_{A}-E_{B}-E_{B}-E_{C}-E_{C}

Multiple repeats of multiple distinct epitopes (repeats of epitopes A, B, and C):

E_{A}-E_{B}-E_{C}-E_{A}-E_{B}-E_{C}-E_{A}-E_{B}-E_{C}; or

E_{A}-E_{A}-E_{A}-E_{B}-E_{B}-E_{B}-E_{C}-E_{C}-E_{C}

The above examples are not limiting and the antigen-encoding nucleic acid sequences having repeats of at least one of the distinct epitopes can encode each of the distinct epitopes in any order or frequency. For example, the order and frequency can be a random arrangement of the distinct epitopes, e.g., in an example with epitopes A, B, and C, by the formula E_{A}-E_{B}-E_{C}-E_{C}-E_{A}-E_{B}-E_{A}-E_{C}-E_{A}-E_{C}-E_{C}-E_{B}.

Also provided for herein is an antigen-encoding cassette, the antigen-encoding cassette having at least one antigen-encoding nucleic acid sequence described, from 5′ to 3′, by the formula:

(Eₓ-(E^{N}ₙ)_{y})_{z}
where E represents a nucleotide sequence comprising at least one of the at least one distinct epitope-encoding nucleic acid sequences,
n represents the number of separate distinct epitope-encoding nucleic acid sequences and is any integer including 0,
E^{N} represents a nucleotide sequence comprising the separate distinct epitope-encoding nucleic acid sequence for each corresponding n,
for each iteration of z: x=0 or 1, y=0 or 1 for each n, and at least one of x or y=1, and z=2 or greater, wherein the antigen-encoding nucleic acid sequence comprises at least two iterations of E, a given E^{N}, or a combination thereof.

Each E or E^{N} can independently comprise any epitope-encoding nucleic acid sequence described herein (e.g., a nucleotide sequence encoding a polypeptide sequence as set forth in Table A, Table B, and/or Table C). For example, Each E or E^{N} can independently comprises a nucleotide sequence described, from 5′ to 3′, by the formula (L5b-Ne-L3d), where N comprises the distinct epitope-encoding nucleic acid sequence associated with each E or E^{N}, where c=1, L5 comprises a 5′ linker sequence, where b=0 or 1, and L3 comprises a 3′ linker sequence, where d=0 or 1. Epitopes and linkers that can be used are further described herein..

Repeats of an epitope-encoding nucleic acid sequences (inclusive of optional 5′ linker sequence and/or the optional 3′ linker sequences) can be linearly linked directly to one another (e.g., E_{A}-E_{A}- . . . as illustrated above). Repeats of an epitope-encoding nucleic acid sequences can be separated by one or more additional nucleotides sequences. In general, repeats of an epitope-encoding nucleic acid sequences can be separated by any size nucleotide sequence applicable for the compositions described herein. In one example, repeats of an epitope-encoding nucleic acid sequences can be separated by a separate distinct epitope-encoding nucleic acid sequence (e.g., E_{A}-E_{B}-E_{C}-E_{A} . . . , as illustrated above). In examples where repeats are separated by a single separate distinct epitope-encoding nucleic acid sequence, and each epitope-encoding nucleic acid sequences (inclusive of optional 5′ linker sequence and/or the optional 3′ linker sequences) encodes a peptide 25 amino acids in length, the repeats can be separated by 75 nucleotides, such as in antigen-encoding nucleic acid represented by E_{A}-E_{B}-E_{A} . . . , E_{A} is separated by 75 nucleotides. In an illustrative example, an antigen-encoding nucleic acid having the sequence VTNTEMFVTAPDNLGYMYEVQWPGQTQPQIANCSVYDFFVWLHYYSVRDTVTNTEMF VTAPDNLGYMYEVQWPGQTQPQIANCSVYDFFVWLHYYSVRDT (SEQ ID NO: 115) encoding repeats of 25mer antigens Trp1 (VTNTEMFVTAPDNLGYMYEVQWPGQ; SEQ ID NO: 116) and Trp2 (TQPQIANCSVYDFFVWLHYYSVRDT; SEQ ID NO: 117), the repeats of Trp1 are separated by the 25mer Trp2 and thus the repeats of the Trp1 epitope-encoding nucleic acid sequences are separated the 75 nucleotide Trp2 epitope-encoding nucleic acid sequence. In examples where repeats are separated by 2, 3, 4, 5, 6, 7, 8, or 9 separate distinct epitope-encoding nucleic acid sequence, and each epitope-encoding nucleic acid sequences (inclusive of optional 5′ linker sequence and/or the optional 3′ linker sequences) encodes a peptide 25 amino acids in length, the repeats can be separated by 150, 225, 300, 375, 450, 525, 600, or 675 nucleotides, respectively.

In one embodiment, different peptides and/or polypeptides or nucleotide sequences encoding them are selected so that the peptides and/or polypeptides capable of associating with different MHC molecules, such as different MHC class I molecules and/or different MHC class II molecules. In some aspects, one vaccine composition comprises coding sequence for peptides and/or polypeptides capable of associating with the most frequently occurring MHC class I molecules and/or different MHC class II molecules. Hence, vaccine compositions can comprise different fragments capable of associating with at least 2 preferred, at least 3 preferred, or at least 4 preferred MHC class I molecules and/or different MHC class II molecules.

The vaccine composition can stimulate a specific cytotoxic T-cell response and a specific helper T-cell response.

The vaccine composition can stimulate a specific B-cell response (e.g., an antibody response).

The vaccine composition can stimulate a specific cytotoxic T-cell response, a specific helper T-cell response, and/or a specific B-cell response. The vaccine composition can stimulate a specific cytotoxic T-cell response and a specific B-cell response. The vaccine composition can stimulate a specific helper T-cell response and a specific B-cell response. The vaccine composition can stimulate a specific cytotoxic T-cell response, a specific helper T-cell response, and a specific B-cell response.

A combination of vaccine compositions can stimulate a specific cytotoxic T-cell response, a specific helper T-cell response, and/or a specific B-cell response. Vaccine compositions can be homologous and stimulate a specific cytotoxic T-cell response, a specific helper T-cell response, and/or a specific B-cell response in combination. Vaccine compositions can be homologous and stimulate a specific cytotoxic T-cell response, a specific helper T-cell response, and a specific B-cell response in combination. Vaccine compositions can be heterologous and stimulate a specific cytotoxic T-cell response, a specific helper T-cell response, and/or a specific B-cell response in combination. Vaccine compositions can be heterologous and stimulate a specific cytotoxic T-cell response, a specific helper T-cell response, and a specific B-cell response in combination. Heterologous vaccines include an identical antigen cassette encoded by different vaccine platforms, e.g., a viral vaccine (e.g., a ChAdV-based platform) and a mRNA vaccine (e.g., a SAM-based platform). Heterologous vaccines include different antigen cassettes (e.g., a Spike cassette and a separate T cell epitope encoding cassette, or epitopes/antigens derived from different subtype isolates of SARS-COV-2, such as Spike protein variants from a Wuhan-Hu-1 subtype isolate and a B.1.351 subtype isolate) encoded by the same vaccine platform, e.g., either a viral vaccine (e.g., a ChAdV-based platform) or a mRNA vaccine (e.g., a SAM-based platform). Heterologous vaccines include different antigen cassettes (e.g., a Spike cassette and a separate T cell epitope encoding cassette or epitopes/antigens derived from different isolate/subtype of coronavirus, such as Spike protein variants from a Wuhan-Hu-1 subtype isolate and a B.1.351 subtype isolate, and/or difference sarbecovirus isolates) encoded by different vaccine platforms, e.g., a viral vaccine (e.g., a ChAdV-based platform) and a mRNA vaccine (e.g., a SAM-based platform). For example, as an illustrative non-limiting example, a viral vaccine (e.g., a ChAdV-based platform) can in particular stimulate a robust cytotoxic T-cell response and a mRNA vaccine (e.g., a SAM-based platform) can in particular stimulate a robust B-cell response.

A vaccine composition can further comprise an adjuvant and/or a carrier. Examples of useful adjuvants and carriers are given herein below. A composition can be associated with a carrier such as e.g. a protein or an antigen-presenting cell such as, e.g., a dendritic cell (DC) capable of presenting the peptide to a T-cell.

Adjuvants are any substance whose admixture into a vaccine composition increases or otherwise modifies the immune response to an antigen. Carriers can be scaffold structures, for example a polypeptide or a polysaccharide, to which an antigen, is capable of being associated. Optionally, adjuvants are conjugated covalently or non-covalently.

The ability of an adjuvant to increase an immune response to an antigen is typically manifested by a significant or substantial increase in an immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th response into a primarily cellular, or Th response.

Suitable adjuvants include, but are not limited to 1018 ISS, alum, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, Lipo Vac, MF59, monophosphoryl lipid A, Montanide IMS 1312, Montanide ISA 206, Montanide ISA 50V, Montanide ISA-51, OK-432, OM-174, OM-197-MP-E_{C}, ONTAK, PepTel vector system, PLG microparticles, resiquimod, SRL172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants such as incomplete Freund's or GM-CSF are useful. Several immunological adjuvants (e.g., MF59) specific for dendritic cells and their preparation have been described previously (Dupuis M, et al., Cell Immunol. 1998; 186(1): 18-27; Allison A C; Dev Biol Stand. 1998; 92:3-11). Also cytokines can be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, specifically incorporated herein by reference in its entirety) and acting as immunoadjuvants (e.g., IL-12) (Gabrilovich D I, et al., J Immunother Emphasis Tumor Immunol. 1996 (6):414-418).

CpG immunostimulatory oligonucleotides have also been reported to enhance the effects of adjuvants in a vaccine setting. Other TLR binding molecules such as RNA binding TLR 7, TLR 8 and/or TLR 9 may also be used.

Other examples of useful adjuvants include, but are not limited to, chemically modified CpGs (e.g. CpR, Idera), Poly(I:C)(e.g. polyi:CI2U), non-CpG bacterial DNA or RNA as well as immunoactive small molecules and antibodies such as cyclophosphamide, sunitinib, bevacizumab, celebrex, NCX-4016, sildenafil, tadalafil, vardenafil, sorafinib, XL-999, CP-547632, pazopanib, ZD2171, AZD2171, ipilimumab, tremelimumab, and SC58175, which may act therapeutically and/or as an adjuvant. The amounts and concentrations of adjuvants and additives can readily be determined by the skilled artisan without undue experimentation. Additional adjuvants include colony-stimulating factors, such as Granulocyte Macrophage Colony Stimulating Factor (GM-CSF, sargramostim).

A vaccine composition can comprise more than one different adjuvant. Furthermore, a therapeutic composition can comprise any adjuvant substance including any of the above or combinations thereof. It is also contemplated that a vaccine and an adjuvant can be administered together or separately in any appropriate sequence.

A carrier (or excipient) can be present independently of an adjuvant. The function of a carrier can for example be to increase the molecular weight of in particular mutant to increase activity or immunogenicity, to confer stability, to increase the biological activity, or to increase serum half-life. Furthermore, a carrier can aid presenting peptides to T-cells. A carrier can be any suitable carrier known to the person skilled in the art, for example a protein or an antigen presenting cell. A carrier protein could be but is not limited to keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier is generally a physiologically acceptable carrier acceptable to humans and safe. However, tetanus toxoid and/or diphtheria toxoid are suitable carriers. Alternatively, the carrier can be dextrans for example Sepharose.

Cytotoxic T-cells (CTLs) recognize an antigen in the form of a peptide bound to an MHC molecule rather than the intact foreign antigen itself. The MHC molecule itself is located at the cell surface of an antigen presenting cell. Thus, an activation of CTLs is possible if a trimeric complex of peptide antigen, MHC molecule, and APC is present. Correspondingly, it may enhance the immune response if not only the peptide is used for activation of CTLs, but if additionally APCs with the respective MHC molecule are added. Therefore, in some embodiments a vaccine composition additionally contains at least one antigen presenting cell.

Antigens can also be included in viral vector-based vaccine platforms, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In Vivo Gene Delivery, J. Virol. (1998) 72 (12): 9873-9880). Dependent on the packaging capacity of the above mentioned viral vector-based vaccine platforms, this approach can deliver one or more nucleotide sequences that encode one or more antigen peptides. The sequences may be flanked by non-mutated sequences, may be separated by linkers or may be preceded with one or more sequences targeting a subcellular compartment (See, e.g., Gros et al., Prospective identification of neoantigen-specific lymphocytes in the peripheral blood of melanoma patients, Nat Med. (2016) 22 (4):433-8, Stronen et al., Targeting of cancer neoantigens with donor-derived T cell receptor repertoires, Science. (2016) 352 (6291): 1337-41, Lu et al., Efficient identification of mutated cancer antigens recognized by T cells associated with durable tumor regressions, Clin Cancer Res. (2014) 20(13):3401-10). Upon introduction into a host, infected cells express the antigens, and thereby stimulate a host immune (e.g., CTL) response against the peptide(s). Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vaccine vectors useful for therapeutic administration or immunization of antigens, e.g., Salmonella typhi vectors, and the like will be apparent to those skilled in the art from the description herein.

### V.A. Antigen Cassette

The methods employed for the selection of one or more antigens, the cloning and construction of a “cassette” and its insertion into a viral vector are within the skill in the art given the teachings provided herein. By “antigen cassette” or “cassette” is meant the combination of a selected antigen or plurality of antigens (e.g., antigen-encoding nucleic acid sequences) and the other regulatory elements necessary to transcribe the antigen(s) and express the transcribed product. The selected antigen or plurality of antigens can refer to distinct epitope sequences, e.g., an antigen-encoding nucleic acid sequence in the cassette can encode an epitope-encoding nucleic acid sequence (or plurality of epitope-encoding nucleic acid sequences) such that the epitopes are transcribed and expressed. An antigen or plurality of antigens can be operatively linked to regulatory components in a manner which permits transcription. Such components include conventional regulatory elements that can drive expression of the antigen(s) in a cell transfected with the viral vector. Thus the antigen cassette can also contain a selected promoter which is linked to the antigen(s) and located, with other, optional regulatory elements, within the selected viral sequences of the recombinant vector. A cassette can have one or more antigen-encoding nucleic acid sequences, such as a cassette containing multiple antigen-encoding nucleic acid sequences each independently operably linked to separate promoters and/or linked together using other multicistonic systems, such as 2A ribosome skipping sequence elements (e.g., E2A, P2A, F2A, or T2A sequences) or Internal Ribosome Entry Site (IRES) sequence elements. A linker can also have a cleavage site, such as a TEV or furin cleavage site. Linkers with cleavage sites can be used in combination with other elements, such as those in a multicistronic system. In a non-limiting illustrative example, a furin protease cleavage site can be used in conjuction with a 2A ribosome skipping sequence element such that the furin protease cleavage site is configured to facilitate removal of the 2A sequence following translation. In a cassette containing more than one antigen-encoding nucleic acid sequences, each antigen-encoding nucleic acid sequence can contain one or more epitope-encoding nucleic acid sequences (e.g., an antigen-encoding nucleic acid sequence encoding concatenated T cell epitopes). In illustrative examples of multicistronic formats, cassettes encoding coronavirus antigens are configured as follows: (1) endogenous 26S promoter-Spike protein-T2A-Membrane protein, or (2) endogenous 26S promoter-Spike protein-26S promoter-concatenated T cell epitopes.

Useful promoters can be constitutive promoters or regulated (inducible) promoters, which will enable control of the amount of antigen(s) to be expressed. For example, a desirable promoter is that of the cytomegalovirus immediate early promoter/enhancer [see, e.g., Boshart et al, Cell, 41:521-530 (1985)]. Another desirable promoter includes the Rous sarcoma virus LTR promoter/enhancer. Still another promoter/enhancer sequence is the chicken cytoplasmic beta-actin promoter [T. A. Kost et al, Nucl. Acids Res., 11(23):8287 (1983)]. Other suitable or desirable promoters can be selected by one of skill in the art.

The antigen cassette can also include nucleic acid sequences heterologous to the viral vector sequences including sequences providing signals for efficient polyadenylation of the transcript (poly(A), poly-A or pA) and introns with functional splice donor and acceptor sites. A common poly-A sequence which is employed in the exemplary vectors of this invention is that derived from the papovavirus SV-40. The poly-A sequence generally can be inserted in the cassette following the antigen-based sequences and before the viral vector sequences. A common intron sequence can also be derived from SV-40, and is referred to as the SV-40 T intron sequence. An antigen cassette can also contain such an intron, located between the promoter/enhancer sequence and the antigen(s). Selection of these and other common vector elements are conventional [see, e.g., Sambrook et al, “Molecular Cloning. A Laboratory Manual.”, 2d edit., Cold Spring Harbor Laboratory, New York (1989) and references cited therein] and many such sequences are available from commercial and industrial sources as well as from Genbank.

An antigen cassette can have one or more antigens. For example, a given cassette can include 1-10, 1-20, 1-30, 10-20, 15-25, 15-20, 1, 2, 3, 4, 5, 6, 7, 8, 9, 10, 11, 12, 13, 14, 15, 16, 17, 18, 19, 20, or more antigens. Antigens can be linked directly to one another. Antigens can also be linked to one another with linkers. Antigens can be in any orientation relative to one another including N to C or C to N.

As described elsewhere, the antigen cassette can be located in the site of any selected deletion in the viral vector backbone, such as the site of the E1 gene region deletion or E3 gene region deletion of a ChAd-based vector or the deleted structural proteins of a VEE backbone, among others which may be selected.

The antigen encoding sequence (e.g., cassette or one or more of the nucleic acid sequences encoding an immunogenic polypeptide in the cassette) can be described using the following formula to describe the ordered sequence of each element, from 5′ to 3′:

Pₐ-(L5_{b}-N_{c}-L3_{d})_{X}-(G5ₑ-U_{f})_{Y}-G3_{g}

wherein P comprises the second promoter nucleotide sequence, where a=0 or 1, where c=1, N comprises one of the coronavirus derived nucleic acid sequences described herein, optionally wherein each N encodes a polypeptide sequence as set forth in Table A, Table B, and/or Table C,L5 comprises the 5′ linker sequence, where b=0 or 1, L3 comprises the 3′ linker sequence, where d=0 or 1, G5 comprises one of the at least one nucleic acid sequences encoding a GPGPG amino acid linker (SEQ ID NO: 56), where e=0 or 1, G3 comprises one of the at least one nucleic acid sequences encoding a GPGPG amino acid linker (SEQ ID NO: 56), where g=0 or 1, U comprises one of the at least one MHC class II epitope-encoding nucleic acid sequence, where f=1, X=1 to 400, where for each X the corresponding Ne is a coronavirus derived nucleic acid sequence, and Y=0, 1, or 2, where for each Y the corresponding Uf is a (1) universal MHC class II epitope-encoding nucleic acid sequence, optionally wherein the at least one universal sequence comprises at least one of Tetanus toxoid and PADRE, or (2) a MHC class II coronavirus derived epitope-encoding nucleic acid sequence. In some aspects, for each X the corresponding Ne is a distinct coronavirus derived nucleic acid sequence. In some aspects, for each Y the corresponding Ur is a distinct universal MHC class II epitope-encoding nucleic acid sequence or a distinct MHC class II coronavirus derived epitope-encoding nucleic acid sequence. The above antigen encoding sequence formula in some instances only describes the portion of an antigen cassette encoding concatenated epitope sequences, such as concatenated T cell epitopes. For example, in cassettes encoding concatenated T cell epitopes and one or more full-length coronavirus proteins, the above antigen encoding sequence formula describes the concatenated T cell epitopes and separately the cassette encodes one or more full-length coronavirus proteins that are linked optionally using a multicistonic system, such as 2A ribosome skipping sequence elements (e.g., E2A, P2A, F2A, or T2A sequences), a Internal Ribosome Entry Site (IRES) sequence elements, and/or independently operably linked to a separate promoter.

In one example, elements present include where b=1, d=1, c=1, g=1, h=1, X=18, Y=2, and the vector backbone comprises a ChAdV68 vector, a=1, P is a CMV promoter, the at least one second poly(A) sequence is present, wherein the second poly(A) sequence is an exogenous poly(A) sequence to the vector backbone, and optionally wherein the exogenous poly(A) sequence comprises an SV40 poly(A) signal sequence or a BGH poly(A) signal sequence, and each N encodes a MHC class I epitope 7-15 amino acids in length, a MHC class II epitope, an epitope capable of stimulating a B cell response, or combinations thereof, L5 is a native 5′ linker sequence that encodes a native N-terminal amino acid sequence of the epitope, and wherein the 5′ linker sequence encodes a peptide that is at least 3 amino acids in length, L3 is a native 3′ linker sequence that encodes a native C-terminal amino acid sequence of the epitope, and wherein the 3′ linker sequence encodes a peptide that is at least 3 amino acids in length, and U is each of a PADRE class II sequence and a Tetanus toxoid MHC class II sequence. The above antigen encoding sequence formula in some instances only describes the portion of an antigen cassette encoding concatenated epitope sequences, such as concatenated T cell epitopes.

In one example, elements present include where b=1, d=1, c=1, g=1, h=1, X=18, Y=2, and the vector backbone comprises a Venezuelan equine encephalitis virus vector, a=0, and the antigen cassette is operably linked to an endogenous 26S promoter, and the at least one polyadenylation poly(A) sequence is a poly(A) sequence of at least 80 consecutive A nucleotides (SEQ ID NO: 27940) provided by the backbone, and each N encodes a MHC class I epitope 7-15 amino acids in length, a MHC class II epitope, an epitope capable of stimulating a B cell response, or combinations thereof, L5 is a native 5′ linker sequence that encodes a native N-terminal amino acid sequence of the epitope, and wherein the 5′ linker sequence encodes a peptide that is at least 3 amino acids in length, L3 is a native 3′ linker sequence that encodes a native C-terminal amino acid sequence of the epitope, and wherein the 3′ linker sequence encodes a peptide that is at least 3 amino acids in length, and U is each of a PADRE class II sequence and a Tetanus toxoid MHC class II sequence.

The antigen encoding sequence can be described using the following formula to describe the ordered sequence of each element, from 5′ to 3′:

(Pₐ-(L5_{b}-N_{c}-L3_{d})_{X})_{Z}-(P2ₕ-(G5ₑ-U_{f})_{Y}_{W}-G3_{g}

wherein P and P2 comprise promoter nucleotide sequences, N comprises one of the coronavirus derived nucleic acid sequences described herein (e.g., N encodes a polypeptide sequence as set forth in Table A, Table B, Table C, and/or Table 7), L5 comprises a 5′ linker sequence, L3 comprises a 3′ linker sequence, G5 comprises a nucleic acid sequences encoding an amino acid linker, G3 comprises one of the at least one nucleic acid sequences encoding an amino acid linker, U comprises an MHC class II epitope-encoding nucleic acid sequence, where for each X the corresponding Nc is a coronavirus derived nucleic acid sequence, where for each Y the corresponding U_{f} is a (1) universal MHC class II epitope-encoding nucleic acid sequence, optionally wherein the at least one universal sequence comprises at least one of Tetanus toxoid and PADRE, or (2) a MHC class II coronavirus derived epitope-encoding nucleic acid sequence. The composition and ordered sequence can be further defined by selecting the number of elements present, for example where a=0 or 1, where b=0 or 1, where c=1, where d=0 or 1, where e=0 or 1, where f=1, where g=0 or 1, where h=0 or 1, X=1 to 400, Y=0, 1, 2, 3, 4 or 5, Z=1 to 400, and W=0, 1, 2, 3, 4 or 5.

In one example, elements present include where a=0, b=1, d=1, c=1, g=1, h=0, X=10, Y=2, Z=1, and W=1, describing where no additional promoter is present (e.g. only the promoter nucleotide sequence provided by the vector backbone, such as an RNA alphavirus backbone, is present), 10 epitopes are present, a 5′ linker is present for each N, a 3′ linker is present for each N, 2 MHC class II epitopes are present, a linker is present linking the two MHC class II epitopes, a linker is present linking the 5′ end of the two MHC class II epitopes to the 3′ linker of the final MHC class I epitope, and a linker is present linking the 3′ end of the two MHC class II epitopes to the to the vector backbone. Examples of linking the 3′ end of the antigen cassette to the vector backbone include linking directly to the 3′ UTR elements provided by the vector backbone, such as a 3′ 19-nt CSE. Examples of linking the 5′ end of the antigen cassette to the vector backbone include linking directly to a promoter or 5′ UTR element of the vector backbone, such as a 26S promoter sequence, an alphavirus 5′ UTR, a 51-nt CSE, or a 24-nt CSE of an alphavirus vector backbone.

Other examples include: where a=1 describing where a promoter other than the promoter nucleotide sequence provided by the vector backbone is present; where a=1 and Z is greater than 1 where multiple promoters other than the promoter nucleotide sequence provided by the vector backbone are present each driving expression of 1 or more distinct MHC class I epitope encoding nucleic acid sequences; where h=1 describing where a separate promoter is present to drive expression of the MHC class II epitope-encoding nucleic acid sequences; and where g=0 describing the MHC class II epitope-encoding nucleic acid sequence, if present, is directly linked to the vector backbone.

Other examples include where each MHC class I epitope that is present can have a 5′ linker, a 3′ linker, neither, or both. In examples where more than one MHC class I epitope is present in the same antigen cassette, some MHC class I epitopes may have both a 5′ linker and a 3′ linker, while other MHC class I epitopes may have either a 5′ linker, a 3′ linker, or neither. In other examples where more than one MHC class I epitope is present in the same antigen cassette, some MHC class I epitopes may have either a 5′ linker or a 3′ linker, while other MHC class I epitopes may have either a 5′ linker, a 3′ linker, or neither.

In examples where more than one MHC class II epitope is present in the same antigen cassette, some MHC class II epitopes may have both a 5′ linker and a 3′ linker, while other MHC class II epitopes may have either a 5′ linker, a 3′ linker, or neither. In other examples where more than one MHC class II epitope is present in the same antigen cassette, some MHC class II epitopes may have either a 5′ linker or a 3′ linker, while other MHC class II epitopes may have either a 5′ linker, a 3′ linker, or neither.

Other examples include where each antigen that is present can have a 5′ linker, a 3′ linker, neither, or both. In examples where more than one antigen is present in the same antigen cassette, some antigens may have both a 5′ linker and a 3′ linker, while other antigens may have either a 5′ linker, a 3′ linker, or neither. In other examples where more than one antigen is present in the same antigen cassette, some antigens may have either a 5′ linker or a 3′ linker, while other antigens may have either a 5′ linker, a 3′ linker, or neither.

The promoter nucleotide sequences P and/or P2 can be the same as a promoter nucleotide sequence provided by the vector backbone, such as a RNA alphavirus backbone. For example, the promoter sequence provided by the vector backbone, Pn and P2, can each comprise a 26S subgenomic promoter or a CMV promoter. The promoter nucleotide sequences P and/or P2 can be different from the promoter nucleotide sequence provided by the vector backbone, as well as can be different from each other.

The 5′ linker L5 can be a native sequence or a non-natural sequence. Non-natural sequence include, but are not limited to, AAY, RR, and DPP. The 3′ linker L3 can also be a native sequence or a non-natural sequence. Additionally, L5 and L3 can both be native sequences, both be non-natural sequences, or one can be native and the other non-natural. For each X, the amino acid linkers can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. For each X, the amino acid linkers can be also be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length.

The amino acid linker G5, for each Y, can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. For each Y, the amino acid linkers can be also be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length.

The amino acid linker G3 can be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids in length. G3 can be also be at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length.

For each X, each N can encode a MHC class I epitope, a MHC class II epitope, an epitope capable of stimulating a B cell response, or a combination thereof. For each X, N can encode a combination of a MHC class I epitope, a MHC class II epitope, and an epitope capable of stimulating a B cell response. For each X, N can encode a combination of a MHC class I epitope and a MHC class II epitope. For each X, N can encode a combination of a MHC class I epitope and an epitope capable of stimulating a B cell response. For each X, N can encode a combination of a MHC class II epitope and an epitope capable of stimulating a B cell response. For each X, each N can encode a MHC class I epitope 7-15 amino acids in length. For each X, each N can also encodes a MHC class I epitope 5, 6, 7, 8, 9, 10, 11, 12, 13, 14, 15, 16, 17, 18, 19, 20, 21, 22, 23, 24, 25, 26, 27, 28, 29, or 30 amino acids in length. For each X, each N can also encodes a MHC class I epitope at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 amino acids in length. For each X, each N can encode a MHC class II epitope. For each X, each N can encode an epitope capable of stimulating a B cell response.

The cassette encoding the one or more antigens can be 700 nucleotides or less. The cassette encoding the one or more antigens can be 700 nucleotides or less and encode 2 distinct epitope-encoding nucleic acid sequences (e.g., encode 2 distinct coronavirus derived nucleic acid sequence encoding an immunogenic polypeptide). The cassette encoding the one or more antigens can be 700 nucleotides or less and encode at least 2 distinct epitope-encoding nucleic acid sequences. The cassette encoding the one or more antigens can be 700 nucleotides or less and encode 3 distinct epitope-encoding nucleic acid sequences. The cassette encoding the one or more antigens can be 700 nucleotides or less and encode at least 3 distinct epitope-encoding nucleic acid sequences. The cassette encoding the one or more antigens can be 700 nucleotides or less and include 1-10, 1-5, 1, 2, 3, 4, 5, 6, 7, 8, 9, 10, or more antigens.

The cassette encoding the one or more antigens can be between 375-700 nucleotides in length. The cassette encoding the one or more antigens can be between 375-700 nucleotides in length and encode 2 distinct epitope-encoding nucleic acid sequences. The cassette encoding the one or more antigens can be between 375-700 nucleotides in length and encode at least 2 distinct epitope-encoding nucleic acid sequences. The cassette encoding the one or more antigens can be between 375-700 nucleotides in length and encode 3 distinct epitope-encoding nucleic acid sequences. The cassette encoding the one or more antigens be between 375-700 nucleotides in length and encode at least 3 distinct epitope-encoding nucleic acid sequences. The cassette encoding the one or more antigens can be between 375-700 nucleotides in length and include 1-10, 1-5, 1, 2, 3, 4, 5, 6, 7, 8, 9, 10, or more antigens.

The cassette encoding the one or more antigens can be 600, 500, 400, 300, 200, or 100 nucleotides in length or less. The cassette encoding the one or more antigens can be 600, 500, 400, 300, 200, or 100 nucleotides in length or less and encode 2 distinct epitope-encoding nucleic acid sequences. The cassette encoding the one or more antigens can be 600, 500, 400, 300, 200, or 100 nucleotides in length or less and encode at least 2 distinct epitope-encoding nucleic acid sequences. The cassette encoding the one or more antigens can be 600, 500, 400, 300, 200, or 100 nucleotides in length or less and encode 3 distinct epitope-encoding nucleic acid sequences. The cassette encoding the one or more antigens can be 600, 500, 400, 300, 200, or 100 nucleotides in length or less and encode at least 3 distinct epitope-encoding nucleic acid sequences. The cassette encoding the one or more antigens can be 600, 500, 400, 300, 200, or 100 nucleotides in length or less and include 1-10, 1-5, 1, 2, 3, 4, 5, 6, 7, 8, 9, 10, or more antigens.

The cassette encoding the one or more antigens can be between 375-600, between 375-500, or between 375-400 nucleotides in length. The cassette encoding the one or more antigens can be between 375-600, between 375-500, or between 375-400 nucleotides in length and encode 2 distinct epitope-encoding nucleic acid sequences. The cassette encoding the one or more antigens can be between 375-600, between 375-500, or between 375-400 nucleotides in length and encode at least 2 distinct epitope-encoding nucleic acid sequences. The cassette encoding the one or more antigens can be between 375-600, between 375-500, or between 375-400 nucleotides in length and encode 3 distinct epitope-encoding nucleic acid sequences. The cassette encoding the one or more antigens can be between 375-600, between 375-500, or between 375-400 nucleotides in length and encode at least 3 distinct epitope-encoding nucleic acid sequences. The cassette encoding the one or more antigens can be between 375-600, between 375-500, or between 375-400 nucleotides in length and include 1-10, 1-5, 1, 2, 3, 4, 5, 6, 7, 8, 9, 10, or more antigens.

### V.B. Additional Considerations for Vaccine Design and Manufacture

After all of the above antigen filters are applied, more candidate antigens may still be available for vaccine inclusion than the vaccine technology can support. Additionally, uncertainty about various aspects of the antigen analysis may remain and tradeoffs may exist between different properties of candidate vaccine antigens. Thus, in place of predetermined filters at each step of the selection process, an integrated multi-dimensional model can be considered that places candidate antigens in a space with at least the following axes and optimizes selection using an integrative approach.
1. Risk of auto-immunity or tolerance (risk of germline) (lower risk of auto-immunity is typically preferred)
2. Probability of sequencing artifact (lower probability of artifact is typically preferred)
3. Probability of immunogenicity (higher probability of immunogenicity is typically preferred)
4. Probability of presentation (higher probability of presentation is typically preferred)
5. Gene expression (higher expression is typically preferred)
6. Coverage of HLA genes (larger number of HLA molecules involved in the presentation of a set of antigens may lower the probability that an infected cell will escape immune attack via downregulation or mutation of HLA molecules)
7. Coverage of HLA classes (covering both HLA-I and HLA-II may increase the probability of therapeutic response and decrease the probability of infectious disease escape)

Additionally, optionally, antigens can be deprioritized (e.g., excluded) from the vaccination if they are predicted to be presented by HLA alleles lost or inactivated in either all or part of the patient's infected cell. HLA allele loss can occur by either somatic mutation, loss of heterozygosity, or homozygous deletion of the locus. Methods for detection of HLA allele somatic mutation are well known in the art, e.g. (Shukla et al., 2015). Methods for detection of somatic LOH and homozygous deletion (including for HLA locus) are likewise well described. (Carter et al., 2012; McGranahan et al., 2017; Van Loo et al., 2010). Antigens can also be deprioritized if mass-spectrometry data indicates a predicted antigen is not presented by a predicted HLA allele.

### V.C. Self-Amplifying RNA Vectors

In general, all self-amplifying RNA (SAM) vectors contain a self-amplifying backbone derived from a self-replicating virus. The term “self-amplifying backbone” refers to minimal sequence(s) of a self-replicating virus that allows for self-replication of the viral genome. For example, minimal sequences that allow for self-replication of an alphavirus can include conserved sequences for nonstructural protein-mediated amplification (e.g., a nonstructural protein 1 (nsP1) gene, a nsP2 gene, a nsP3 gene, a nsP4 gene, and/or a polyA sequence). A self-amplifying backbone can also include sequences for expression of subgenomic viral RNA (e.g., a 26S promoter element for an alphavirus). SAM vectors can be positive-sense RNA polynucleotides or negative-sense RNA polynucleotides, such as vectors with backbones derived from positive-sense or negative-sense self-replicating viruses. Self-replicating viruses include, but are not limited to, alphaviruses, flaviviruses (e.g., Kunjin virus), measles viruses, and rhabdoviruses (e.g., rabies virus and vesicular stomatitis virus). Examples of SAM vector systems derived from self-replicating viruses are described in greater detail in Lundstrom (Molecules. 2018 Dec. 13; 23(12). pii: E3310. doi: 10.3390/molecules23123310), herein incorporated by reference for all purposes.

### V.C.1. Alphavirus Biology

Alphaviruses are members of the family Togaviridae, and are positive-sense single stranded RNA viruses. Members are typically classified as either Old World, such as Sindbis, Ross River, Mayaro, Chikungunya, and Semliki Forest viruses, or New World, such as eastern equine encephalitis, Aura, Fort Morgan, or Venezuelan equine encephalitis virus and its derivative strain TC-83 (Strauss Microbrial Review 1994). A natural alphavirus genome is typically around 12 kb in length, the first two-thirds of which contain genes encoding non-structural proteins (nsPs) that form RNA replication complexes for self-replication of the viral genome, and the last third of which contains a subgenomic expression cassette encoding structural proteins for virion production (Frolov RNA 2001).

A model lifecycle of an alphavirus involves several distinct steps (Strauss Microbrial Review 1994, Jose Future Microbiol 2009). Following virus attachment to a host cell, the virion fuses with membranes within endocytic compartments resulting in the eventual release of genomic RNA into the cytosol. The genomic RNA, which is in a plus-strand orientation and comprises a 5′ methylguanylate cap and 3′ polyA tail, is translated to produce non-structural proteins nsP1-4 that form the replication complex. Early in infection, the plus-strand is then replicated by the complex into a minus-stand template. In the current model, the replication complex is further processed as infection progresses, with the resulting processed complex switching to transcription of the minus-strand into both full-length positive-strand genomic RNA, as well as the 26S subgenomic positive-strand RNA containing the structural genes. Several conserved sequence elements (CSEs) of alphavirus have been identified to potentially play a role in the various RNA replication steps including; a complement of the 5′ UTR in the replication of plus-strand RNAs from a minus-strand template, a 51-nt CSE in the replication of minus-strand synthesis from the genomic template, a 24-nt CSE in the junction region between the nsPs and the 26S RNA in the transcription of the subgenomic RNA from the minus-strand, and a 3′ 19-nt CSE in minus-strand synthesis from the plus-strand template.

Following the replication of the various RNA species, virus particles are then typically assembled in the natural lifecycle of the virus. The 26S RNA is translated and the resulting proteins further processed to produce the structural proteins including capsid protein, glycoproteins E1 and E2, and two small polypeptides E3 and 6K (Strauss 1994). Encapsidation of viral RNA occurs, with capsid proteins normally specific for only genomic RNA being packaged, followed by virion assembly and budding at the membrane surface.

### V.C.2. Alphavirus as a Delivery Vector

Alphaviruses (including alphavirus sequences, features, and other elements) can be used to generate alphavirus-based delivery vectors (also be referred to as alphavirus vectors, alphavirus viral vectors, alphavirus vaccine vectors, self-replicating RNA (srRNA) vectors, or self-amplifying RNA (samRNA) vectors). Alphaviruses have previously been engineered for use as expression vector systems (Pushko 1997, Rheme 2004). Alphaviruses offer several advantages, particularly in a vaccine setting where heterologous antigen expression can be desired. Due to its ability to self-replicate in the host cytosol, alphavirus vectors are generally able to produce high copy numbers of the expression cassette within a cell resulting in a high level of heterologous antigen production. Additionally, the vectors are generally transient, resulting in improved biosafety as well as reduced induction of immunological tolerance to the vector. The public, in general, also lacks pre-existing immunity to alphavirus vectors as compared to other standard viral vectors, such as human adenovirus. Alphavirus based vectors also generally result in cytotoxic responses to infected cells. Cytotoxicity, to a certain degree, can be important in a vaccine setting to properly illicit an immune response to the heterologous antigen expressed. However, the degree of desired cytotoxicity can be a balancing act, and thus several attenuated alphaviruses have been developed, including the TC-83 strain of VEE. Thus, an example of an antigen expression vector described herein can utilize an alphavirus backbone that allows for a high level of antigen expression, stimulates a robust immune response to antigen, does not stimulate an immune response to the vector itself, and can be used in a safe manner. Furthermore, the antigen expression cassette can be designed to stimulate different levels of an immune response through optimization of which alphavirus sequences the vector uses, including, but not limited to, sequences derived from VEE or its attenuated derivative TC-83.

Several expression vector design strategies have been engineered using alphavirus sequences (Pushko 1997). In one strategy, a alphavirus vector design includes inserting a second copy of the 26S promoter sequence elements downstream of the structural protein genes, followed by a heterologous gene (Frolov 1993). Thus, in addition to the natural non-structural and structural proteins, an additional subgenomic RNA is produced that expresses the heterologous protein. In this system, all the elements for production of infectious virions are present and, therefore, repeated rounds of infection of the expression vector in non-infected cells can occur.

Another expression vector design makes use of helper virus systems (Pushko 1997). In this strategy, the structural proteins are replaced by a heterologous gene. Thus, following self-replication of viral RNA mediated by still intact non-structural genes, the 26S subgenomic RNA provides for expression of the heterologous protein. Traditionally, additional vectors that expresses the structural proteins are then supplied in trans, such as by co-transfection of a cell line, to produce infectious virus. A system is described in detail in U.S. Pat. No. 8,093,021, which is herein incorporated by reference in its entirety, for all purposes. The helper vector system provides the benefit of limiting the possibility of forming infectious particles and, therefore, improves biosafety. In addition, the helper vector system reduces the total vector length, potentially improving the replication and expression efficiency. Thus, an example of an antigen expression vector described herein can utilize an alphavirus backbone wherein the structural proteins are replaced by an antigen cassette, the resulting vector both reducing biosafety concerns, while at the same time promoting efficient expression due to the reduction in overall expression vector size.

### V.C.3. Self-Amplifying Virus Production In Vitro

A convenient technique well-known in the art for RNA production is in vitro transcription (IVT). In this technique, a DNA template of the desired vector is first produced by techniques well-known to those in the art, including standard molecular biology techniques such as cloning, restriction digestion, ligation, gene synthesis, and polymerase chain reaction (PCR).

The DNA template contains a RNA polymerase promoter at the 5′ end of the sequence desired to be transcribed into RNA (e.g., SAM). Promoters include, but are not limited to, bacteriophage polymerase promoters such as T3, T7, K11, or SP6. Depending on the specific RNA polymerase promoter sequence chosen, additional 5′ nucleotides can transcribed in addition to the desired sequence. For example, the canonical T7 promoter can be referred to by the sequence TAATACGACTCACTATAGG (SEQ ID NO: 118), in which an IVT reaction using the DNA template TAATACGACTCACTATAGGN (SEQ ID NO: 119) for the production of desired sequence N will result in the mRNA sequence GG-N. In general, and without wishing to be bound by theory, T7 polymerase more efficiently transcribes RNA transcripts beginning with guanosine. In instances where additional 5′ nucleotides are not desired (e.g., no additional GG), the RNA polymerase promoter contained in the DNA template can be a sequence the results in transcripts containing only the 5′ nucleotides of the desired sequence, e.g., a SAM having the native 5′ sequence of the self-replicating virus from which the SAM vector is derived. For example, a minimal T7 promoter can be referred to by the sequence TAATACGACTCACTATA (SEQ ID NO: 120), in which an IVT reaction using the DNA template TAATACGACTCACTATAN (SEQ ID NO: 121) for the production of desired sequence N will result in the mRNA sequence N. Likewise, a minimal SP6 promoter referred to by the sequence ATTTAGGTGACACTATA (SEQ ID NO: 122) can be used to generate transcripts without additional 5′ nucleotides. In a typical IVT reaction, the DNA template is incubated with the appropriate RNA polymerase enzyme, buffer agents, and nucleotides (NTPs).

The resulting RNA polynucleotide can optionally be further modified including, but limited to, addition of a 5′ cap structure such as 7-methylguanosine or a related structure, and optionally modifying the 3′ end to include a polyadenylate (polyA) tail. In a modified IVT reaction, RNA is capped with a 5′ cap structure co-transcriptionally through the addition of cap analogues during IVT. Cap analogues can include dinucleotide (m⁷G-ppp-N) cap analogues or trinucleotide (m⁷G-ppp-N—N) cap analogues, where N represents a nucleotide or modified nucleotide (e.g., ribonucleosides including, but not limited to, adenosine, guanosine, cytidine, and uradine). Exemplary cap analogues and their use in IVT reactions are also described in greater detail in U.S. Pat. No. 10,519,189, herein incorporated by reference for all purposes. As discussed, T7 polymerase more efficiently transcribes RNA transcripts beginning with guanosine. To improve transcription efficiency in templates that do not begin with guanosine, a trinucleotide cap analogue (m⁷G-ppp-N—N) can be used. The trinucleotide cap analogue can increase transcription efficiency 2, 3, 4, 5, 6, 7, 8, 9, 10, 11, 12, 13, 14, 15, 16, 17, 18, 19, 20-fold or more relative to an IVT reaction using a dinucleotide cap analogue (m7G-ppp-N).

A 5′ cap structure can also be added following transcription, such as using a vaccinia capping system (e.g., NEB Cat. No. M2080) containing mRNA 2′-O-methyltransferase and S-Adenosyl methionine.

The resulting RNA polynucleotide can optionally be further modified separately from or in addition to the capping techniques described including, but limited to, modifying the 3′ end to include a polyadenylate (polyA) tail.

The RNA can then be purified using techniques well-known in the field, such as phenol-chloroform extraction.

### V.C.4. Delivery Via Lipid Nanoparticle

An important aspect to consider in vaccine vector design is immunity against the vector itself (Riley 2017). This may be in the form of preexisting immunity to the vector itself, such as with certain human adenovirus systems, or in the form of developing immunity to the vector following administration of the vaccine. The latter is an important consideration if multiple administrations of the same vaccine are performed, such as separate priming and boosting doses, or if the same vaccine vector system is to be used to deliver different antigen cassettes.

In the case of alphavirus vectors, the standard delivery method is the previously discussed helper virus system that provides capsid, E1, and E2 proteins in trans to produce infectious viral particles. However, it is important to note that the E1 and E2 proteins are often major targets of neutralizing antibodies (Strauss 1994). Thus, the efficacy of using alphavirus vectors to deliver antigens of interest to target cells may be reduced if infectious particles are targeted by neutralizing antibodies.

An alternative to viral particle mediated gene delivery is the use of nanomaterials to deliver expression vectors (Riley 2017). Nanomaterial vehicles, importantly, can be made of non-immunogenic materials and generally avoid stimulating immunity to the delivery vector itself. These materials can include, but are not limited to, lipids, inorganic nanomaterials, and other polymeric materials. Lipids can be cationic, anionic, or neutral. The materials can be synthetic or naturally derived, and in some instances biodegradable. Lipids can include fats, cholesterol, phospholipids, lipid conjugates including, but not limited to, polyethyleneglycol (PEG) conjugates (PEGylated lipids), waxes, oils, glycerides, and fat soluble vitamins.

Lipid nanoparticles (LNPs) are an attractive delivery system due to the amphiphilic nature of lipids enabling formation of membranes and vesicle like structures (Riley 2017). In general, these vesicles deliver the expression vector by absorbing into the membrane of target cells and releasing nucleic acid into the cytosol. In addition, LNPs can be further modified or functionalized to facilitate targeting of specific cell types. Another consideration in LNP design is the balance between targeting efficiency and cytotoxicity. Lipid compositions generally include defined mixtures of cationic, neutral, anionic, and amphipathic lipids. In some instances, specific lipids are included to prevent LNP aggregation, prevent lipid oxidation, or provide functional chemical groups that facilitate attachment of additional moieties. Lipid composition can influence overall LNP size and stability. In an example, the lipid composition comprises dilinoleylmethyl-4-dimethylaminobutyrate (MC3) or MC3-like molecules. MC3 and MC3-like lipid compositions can be formulated to include one or more other lipids, such as a PEG or PEG-conjugated lipid, a sterol, or neutral lipids.

Nucleic-acid vectors, such as expression vectors, exposed directly to serum can have several undesirable consequences, including degradation of the nucleic acid by serum nucleases or off-target stimulation of the immune system by the free nucleic acids. Therefore, encapsulation of the alphavirus vector can be used to avoid degradation, while also avoiding potential off-target effects. In certain examples, an alphavirus vector is fully encapsulated within the delivery vehicle, such as within the aqueous interior of an LNP. Encapsulation of the alphavirus vector within an LNP can be carried out by techniques well-known to those skilled in the art, such as microfluidic mixing and droplet generation carried out on a microfluidic droplet generating device. Such devices include, but are not limited to, standard T-junction devices or flow-focusing devices. In an example, the desired lipid formulation, such as MC3 or MC3-like containing compositions, is provided to the droplet generating device in parallel with the alphavirus delivery vector and other desired agents, such that the delivery vector and desired agents are fully encapsulated within the interior of the MC3 or MC3-like based LNP. In an example, the droplet generating device can control the size range and size distribution of the LNPs produced. For example, the LNP can have a size ranging from 1 to 1000 nanometers in diameter, e.g., 1, 10, 50, 100, 500, or 1000 nanometers. Following droplet generation, the delivery vehicles encapsulating the expression vectors can be further treated or modified to prepare them for administration.

### V.D. Chimpanzee Adenovirus (ChAd)

V.D.1. Viral Delivery with Chimpanzee Adenovirus

Vaccine compositions for delivery of one or more antigens (e.g., via an antigen cassette) can be created by providing adenovirus nucleotide sequences of chimpanzee origin, a variety of novel vectors, and cell lines expressing chimpanzee adenovirus genes. A nucleotide sequence of a chimpanzee C68 adenovirus (also referred to herein as ChAdV68) can be used in a vaccine composition for antigen delivery (See SEQ ID NO: 1). Use of C68 adenovirus derived vectors is described in further detail in U.S. Pat. No. 6,083,716, which is herein incorporated by reference in its entirety, for all purposes.

In a further aspect, provided herein is a recombinant adenovirus comprising the DNA sequence of a chimpanzee adenovirus such as C68 and an antigen cassette operatively linked to regulatory sequences directing its expression. The recombinant virus is capable of infecting a mammalian, preferably a human, cell and capable of expressing the antigen cassette product in the cell. In this vector, the native chimpanzee E1 gene, and/or E3 gene, and/or E4 gene can be deleted. An antigen cassette can be inserted into any of these sites of gene deletion. The antigen cassette can include an antigen against which a primed immune response is desired.

In another aspect, provided herein is a mammalian cell infected with a chimpanzee adenovirus such as C68.

In still a further aspect, a novel mammalian cell line is provided which expresses a chimpanzee adenovirus gene (e.g., from C68) or functional fragment thereof.

In still a further aspect, provided herein is a method for delivering an antigen cassette into a mammalian cell comprising the step of introducing into the cell an effective amount of a chimpanzee adenovirus, such as C68, that has been engineered to express the antigen cassette.

Still another aspect provides a method for stimulating an immune response in a mammalian host. The method can comprise the step of administering to the host an effective amount of a recombinant chimpanzee adenovirus, such as C68, comprising an antigen cassette that encodes one or more antigens from the infection against which the immune response is targeted.

Still another aspect provides a method for stimulating an immune response in a mammalian host to treat or prevent a disease in a subject, such as an infectious disease. The method can comprise the step of administering to the host an effective amount of a recombinant chimpanzee adenovirus, such as C68, comprising an antigen cassette that encodes one or more antigens, such as from the infectious disease against which the immune response is targeted.

Also disclosed is a non-simian mammalian cell that expresses a chimpanzee adenovirus gene obtained from the sequence of SEQ ID NO: 1. The gene can be selected from the group consisting of the adenovirus E1A, E1B, E2A, E2B, E3, E4, L1, L2, L3, L4 and L5 of SEQ ID NO: 1.

Also disclosed is a nucleic acid molecule comprising a chimpanzee adenovirus DNA sequence comprising a gene obtained from the sequence of SEQ ID NO: 1. The gene can be selected from the group consisting of said chimpanzee adenovirus E1A, E1B, E2A, E2B, E3, E4, L1, L2, L3, L4 and L5 genes of SEQ ID NO: 1. In some aspects the nucleic acid molecule comprises SEQ ID NO: 1. In some aspects the nucleic acid molecule comprises the sequence of SEQ ID NO: 1, lacking at least one gene selected from the group consisting of E1A, E1B, E2A, E2B, E3, E4, L1, L2, L3, L4 and L5 genes of SEQ ID NO: 1.

Also disclosed is a vector comprising a chimpanzee adenovirus DNA sequence obtained from SEQ ID NO: 1 and an antigen cassette operatively linked to one or more regulatory sequences which direct expression of the cassette in a heterologous host cell, optionally wherein the chimpanzee adenovirus DNA sequence comprises at least the cis-elements necessary for replication and virion encapsidation, the cis-elements flanking the antigen cassette and regulatory sequences. In some aspects, the chimpanzee adenovirus DNA sequence comprises a gene selected from the group consisting of E1A, E1B, E2A, E2B, E3, E4, L1, L2, L3, L4 and L5 gene sequences of SEQ ID NO: 1. In some aspects the vector can lack the E1A and/or E1B gene.

Also disclosed herein is a adenovirus vector comprising: a partially deleted E4 gene comprising a deleted or partially-deleted E4orf2 region and a deleted or partially-deleted E4orf3 region, and optionally a deleted or partially-deleted E4orf4 region. The partially deleted E4 can comprise an E4 deletion of at least nucleotides 34,916 to 35,642 of the sequence shown in SEQ ID NO:1, and wherein the vector comprises at least nucleotides 2 to 36,518 of the sequence set forth in SEQ ID NO:1. The partially deleted E4 can comprise an E4 deletion of at least a partial deletion of nucleotides 34,916 to 34,942 of the sequence shown in SEQ ID NO: 1, at least a partial deletion of nucleotides 34,952 to 35,305 of the sequence shown in SEQ ID NO:1, and at least a partial deletion of nucleotides 35,302 to 35,642 of the sequence shown in SEQ ID NO:1, and wherein the vector comprises at least nucleotides 2 to 36,518 of the sequence set forth in SEQ ID NO:1 The partially deleted E4 can comprise an E4 deletion of at least nucleotides 34,980 to 36,516 of the sequence shown in SEQ ID NO:1, and wherein the vector comprises at least nucleotides 2 to 36,518 of the sequence set forth in SEQ ID NO:1. The partially deleted E4 can comprise an E4 deletion of at least nucleotides 34,979 to 35,642 of the sequence shown in

SEQ ID NO: 1, and wherein the vector comprises at least nucleotides 2 to 36,518 of the sequence set forth in SEQ ID NO:1. The partially deleted E4 can comprise an E4 deletion of at least a partial deletion of E4Orf2, a fully deleted E4Orf3, and at least a partial deletion of E4Orf4. The partially deleted E4 can comprise an E4 deletion of at least a partial deletion of E4Orf2, at least a partial deletion of E4Orf3, and at least a partial deletion of E4Orf4. The partially deleted E4 can comprise an E4 deletion of at least a partial deletion of E4Orf1, a fully deleted E4Orf2, and at least a partial deletion of E4Orf3. The partially deleted E4 can comprise an E4 deletion of at least a partial deletion of E4Orf2 and at least a partial deletion of E4Orf3. The partially deleted E4 can comprise an E4 deletion between the start site of E4Orf1 to the start site of E4Orf5. The partially deleted E4 can be an E4 deletion adjacent to the start site of E4Orf1. The partially deleted E4 can be an E4 deletion adjacent to the start site of E4Orf2. The partially deleted E4 can be an E4 deletion adjacent to the start site of E4Orf3. The partially deleted E4 can be an E4 deletion adjacent to the start site of E4Orf4. The E4 deletion can be at least 50, at least 100, at least 200, at least 300, at least 400, at least 500, at least 600, at least 700, at least 800, at least 900, at least 1000, at least 1100, at least 1200, at least 1300, at least 1400, at least 1500, at least 1600, at least 1700, at least 1800, at least 1900, or at least 2000 nucleotides. The E4 deletion can be at least 700 nucleotides. The E4 deletion can be at least 1500 nucleotides. The E4 deletion can be 50 or less, 100 or less, 200 or less, 300 or less, 400 or less, 500 or less, 600 or less, 700 or less, 800 or less, 900 or less, 1000 or less, 1100 or less, 1200 or less, 1300 or less, 1400 or less, 1500 or less, 1600 or less, 1700 or less, 1800 or less, 1900 or less, or 2000 or less nucleotides. The E4 deletion can be 750 nucleotides or less. The E4 deletion can be at least 1550 nucleotides or less.

Also disclosed herein is a host cell transfected with a vector disclosed herein such as a C68 vector engineered to expression an antigen cassette. Also disclosed herein is a human cell that expresses a selected gene introduced therein through introduction of a vector disclosed herein into the cell.

Also disclosed herein is a method for delivering an antigen cassette to a mammalian cell comprising introducing into said cell an effective amount of a vector disclosed herein such as a C68 vector engineered to expression the antigen cassette.

Also disclosed herein is a method for producing an antigen comprising introducing a vector disclosed herein into a mammalian cell, culturing the cell under suitable conditions and producing the antigen.

### V.D.2. E1-Expressing Complementation Cell Lines

To generate recombinant chimpanzee adenoviruses (Ad) deleted in any of the genes described herein, the function of the deleted gene region, if essential to the replication and infectivity of the virus, can be supplied to the recombinant virus by a helper virus or cell line, i.e., a complementation or packaging cell line. For example, to generate a replication-defective chimpanzee adenovirus vector, a cell line can be used which expresses the E1 gene products of the human or chimpanzee adenovirus; such a cell line can include HEK293 or variants thereof. The protocol for the generation of the cell lines expressing the chimpanzee E1 gene products (Examples 3 and 4 of U.S. Pat. No. 6,083,716) can be followed to generate a cell line which expresses any selected chimpanzee adenovirus gene.

An AAV augmentation assay can be used to identify a chimpanzee adenovirus E1-expressing cell line. This assay is useful to identify E1 function in cell lines made by using the E1 genes of other uncharacterized adenoviruses, e.g., from other species. That assay is described in Example 4B of U.S. Pat. No. 6,083,716.

A selected chimpanzee adenovirus gene, e.g., E1, can be under the transcriptional control of a promoter for expression in a selected parent cell line. Inducible or constitutive promoters can be employed for this purpose. Among inducible promoters are included the sheep metallothionine promoter, inducible by zinc, or the mouse mammary tumor virus (MMTV) promoter, inducible by a glucocorticoid, particularly, dexamethasone. Other inducible promoters, such as those identified in International patent application WO95/13392, incorporated by reference herein can also be used in the production of packaging cell lines. Constitutive promoters in control of the expression of the chimpanzee adenovirus gene can be employed also.

A parent cell can be selected for the generation of a novel cell line expressing any desired C68 gene. Without limitation, such a parent cell line can be HeLa [ATCC Accession No. CCL 2], A549 [ATCC Accession No. CCL 185], KB [CCL 17], Detroit [e.g., Detroit 510, CCL 72] and WI-38 [CCL 75] cells. Other suitable parent cell lines can be obtained from other sources. Parent cell lines can include CHO, HEK293 or variants thereof, 911, HeLa, A549, LP-293, PER.C6, or AE1-2a.

An E1-expressing cell line can be useful in the generation of recombinant chimpanzee adenovirus E1 deleted vectors. Cell lines constructed using essentially the same procedures that express one or more other chimpanzee adenoviral gene products are useful in the generation of recombinant chimpanzee adenovirus vectors deleted in the genes that encode those products. Further, cell lines which express other human Ad E1 gene products are also useful in generating chimpanzee recombinant Ads.

### V.D.3. Recombinant Viral Particles as Vectors

The compositions disclosed herein can comprise viral vectors, that deliver at least one antigen to cells. Such vectors comprise a chimpanzee adenovirus DNA sequence such as C68 and an antigen cassette operatively linked to regulatory sequences which direct expression of the cassette. The C68 vector is capable of expressing the cassette in an infected mammalian cell. The C68 vector can be functionally deleted in one or more viral genes. An antigen cassette comprises at least one antigen under the control of one or more regulatory sequences such as a promoter. Optional helper viruses and/or packaging cell lines can supply to the chimpanzee viral vector any necessary products of deleted adenoviral genes.

The term “functionally deleted” means that a sufficient amount of the gene region is removed or otherwise altered, e.g., by mutation or modification, so that the gene region is no longer capable of producing one or more functional products of gene expression. Mutations or modifications that can result in functional deletions include, but are not limited to, nonsense mutations such as introduction of premature stop codons and removal of canonical and non-canonical start codons, mutations that alter mRNA splicing or other transcriptional processing, or combinations thereof. If desired, the entire gene region can be removed.

Modifications of the nucleic acid sequences forming the vectors disclosed herein, including sequence deletions, insertions, and other mutations may be generated using standard molecular biological techniques and are within the scope of this invention.

### V.D.4. Construction of the Viral Plasmid Vector

The chimpanzee adenovirus C68 vectors useful in this invention include recombinant, defective adenoviruses, that is, chimpanzee adenovirus sequences functionally deleted in the E1a or E1b genes, and optionally bearing other mutations, e.g., temperature-sensitive mutations or deletions in other genes. It is anticipated that these chimpanzee sequences are also useful in forming hybrid vectors from other adenovirus and/or adeno-associated virus sequences. Homologous adenovirus vectors prepared from human adenoviruses are described in the published literature [see, for example, Kozarsky I and II, cited above, and references cited therein, U.S. Pat. No. 5,240,846].

In the construction of useful chimpanzee adenovirus C68 vectors for delivery of an antigen cassette to a human (or other mammalian) cell, a range of adenovirus nucleic acid sequences can be employed in the vectors. A vector comprising minimal chimpanzee C68 adenovirus sequences can be used in conjunction with a helper virus to produce an infectious recombinant virus particle. The helper virus provides essential gene products required for viral infectivity and propagation of the minimal chimpanzee adenoviral vector. When only one or more selected deletions of chimpanzee adenovirus genes are made in an otherwise functional viral vector, the deleted gene products can be supplied in the viral vector production process by propagating the virus in a selected packaging cell line that provides the deleted gene functions in trans.

### V.D.5. Recombinant Minimal Adenovirus

A minimal chimpanzee Ad C68 virus is a viral particle containing just the adenovirus cis-elements necessary for replication and virion encapsidation. That is, the vector contains the cis-acting 5′ and 3′ inverted terminal repeat (ITR) sequences of the adenoviruses (which function as origins of replication) and the native 5′ packaging/enhancer domains (that contain sequences necessary for packaging linear Ad genomes and enhancer elements for the E1 promoter). See, for example, the techniques described for preparation of a “minimal” human Ad vector in International Patent Application WO96/13597 and incorporated herein by reference.

### V.D.6. Other Defective Adenoviruses

Recombinant, replication-deficient adenoviruses can also contain more than the minimal chimpanzee adenovirus sequences. These other Ad vectors can be characterized by deletions of various portions of gene regions of the virus, and infectious virus particles formed by the optional use of helper viruses and/or packaging cell lines.

As one example, suitable vectors may be formed by deleting all or a sufficient portion of the C68 adenoviral immediate early gene E1a and delayed early gene E1b, so as to eliminate their normal biological functions. Replication-defective E1-deleted viruses are capable of replicating and producing infectious virus when grown on a chimpanzee adenovirus-transformed, complementation cell line containing functional adenovirus E1a and E1b genes which provide the corresponding gene products in trans. Based on the homologies to known adenovirus sequences, it is anticipated that, as is true for the human recombinant E1-deleted adenoviruses of the art, the resulting recombinant chimpanzee adenovirus is capable of infecting many cell types and can express antigen(s), but cannot replicate in most cells that do not carry the chimpanzee E1 region DNA unless the cell is infected at a very high multiplicity of infection.

As another example, all or a portion of the C68 adenovirus delayed early gene E3 can be eliminated from the chimpanzee adenovirus sequence which forms a part of the recombinant virus.

Chimpanzee adenovirus C68 vectors can also be constructed having a deletion of the E4 gene. Still another vector can contain a deletion in the delayed early gene E2a.

Deletions can also be made in any of the late genes L1 through L5 of the chimpanzee C68 adenovirus genome. Similarly, deletions in the intermediate genes IX and IVa2 can be useful for some purposes. Other deletions may be made in the other structural or non-structural adenovirus genes.

The above discussed deletions can be used individually, i.e., an adenovirus sequence can contain deletions of E1 only. Alternatively, deletions of entire genes or portions thereof effective to destroy or reduce their biological activity can be used in any combination. For example, in one exemplary vector, the adenovirus C68 sequence can have deletions of the E1 genes and the E4 gene, or of the E1, E2a and E3 genes, or of the E1 and E3 genes, or of E1, E2a and E4 genes, with or without deletion of E3, and so on. As discussed above, such deletions can be used in combination with other mutations, such as temperature-sensitive mutations, to achieve a desired result.

The cassette comprising antigen(s) be inserted optionally into any deleted region of the chimpanzee C68 Ad virus. Alternatively, the cassette can be inserted into an existing gene region to disrupt the function of that region, if desired.

### V.D.7. Helper Viruses

Depending upon the chimpanzee adenovirus gene content of the viral vectors employed to carry the antigen cassette, a helper adenovirus or non-replicating virus fragment can be used to provide sufficient chimpanzee adenovirus gene sequences to produce an infective recombinant viral particle containing the cassette.

Useful helper viruses contain selected adenovirus gene sequences not present in the adenovirus vector construct and/or not expressed by the packaging cell line in which the vector is transfected. A helper virus can be replication-defective and contain a variety of adenovirus genes in addition to the sequences described above. The helper virus can be used in combination with the E1-expressing cell lines described herein.

For C68, the “helper” virus can be a fragment formed by clipping the C terminal end of the C68 genome with SspI, which removes about 1300 bp from the left end of the virus. This clipped virus is then co-transfected into an E1-expressing cell line with the plasmid DNA, thereby forming the recombinant virus by homologous recombination with the C68 sequences in the plasmid.

Helper viruses can also be formed into poly-cation conjugates as described in Wu et al, J. Biol. Chem., 264: 16985-16987 (1989); K. J. Fisher and J. M. Wilson, Biochem. J., 299:49 (Apr. 1, 1994). Helper virus can optionally contain a reporter gene. A number of such reporter genes are known to the art. The presence of a reporter gene on the helper virus which is different from the antigen cassette on the adenovirus vector allows both the Ad vector and the helper virus to be independently monitored. This second reporter is used to enable separation between the resulting recombinant virus and the helper virus upon purification.

### V.D.8. Assembly of Viral Particle and Infection of a Cell Line

Assembly of the selected DNA sequences of the adenovirus, the antigen cassette, and other vector elements into various intermediate plasmids and shuttle vectors, and the use of the plasmids and vectors to produce a recombinant viral particle can all be achieved using conventional techniques. Such techniques include conventional cloning techniques of cDNA, in vitro recombination techniques (e.g., Gibson assembly), use of overlapping oligonucleotide sequences of the adenovirus genomes, polymerase chain reaction, and any suitable method which provides the desired nucleotide sequence. Standard transfection and co-transfection techniques are employed, e.g., CaPO4 precipitation techniques or liposome-mediated transfection methods such as lipofectamine. Other conventional methods employed include homologous recombination of the viral genomes, plaguing of viruses in agar overlay, methods of measuring signal generation, and the like.

For example, following the construction and assembly of the desired antigen cassette-containing viral vector, the vector can be transfected in vitro in the presence of a helper virus into the packaging cell line. Homologous recombination occurs between the helper and the vector sequences, which permits the adenovirus-antigen sequences in the vector to be replicated and packaged into virion capsids, resulting in the recombinant viral vector particles.

The resulting recombinant chimpanzee C68 adenoviruses are useful in transferring an antigen cassette to a selected cell. In in vivo experiments with the recombinant virus grown in the packaging cell lines, the E1-deleted recombinant chimpanzee adenovirus demonstrates utility in transferring a cassette to a non-chimpanzee, preferably a human, cell.

### V.D.9. Use of the Recombinant Virus Vectors

The resulting recombinant chimpanzee C68 adenovirus containing the antigen cassette (produced by cooperation of the adenovirus vector and helper virus or adenoviral vector and packaging cell line, as described above) thus provides an efficient gene transfer vehicle which can deliver antigen(s) to a subject in vivo or ex vivo.

The above-described recombinant vectors are administered to humans according to published methods for gene therapy. A chimpanzee viral vector bearing an antigen cassette can be administered to a patient, preferably suspended in a biologically compatible solution or pharmaceutically acceptable delivery vehicle. A suitable vehicle includes sterile saline. Other aqueous and non-aqueous isotonic sterile injection solutions and aqueous and non-aqueous sterile suspensions known to be pharmaceutically acceptable carriers and well known to those of skill in the art may be employed for this purpose.

The chimpanzee adenoviral vectors are administered in sufficient amounts to transduce the human cells and to provide sufficient levels of antigen transfer and expression to provide a therapeutic benefit without undue adverse or with medically acceptable physiological effects, which can be determined by those skilled in the medical arts. Conventional and pharmaceutically acceptable routes of administration include, but are not limited to, direct delivery to the liver, intranasal, intravenous, intramuscular, subcutaneous, intradermal, oral and other parental routes of administration. Routes of administration may be combined, if desired.

Dosages of the viral vector will depend primarily on factors such as the condition being treated, the age, weight and health of the patient, and may thus vary among patients. The dosage will be adjusted to balance the therapeutic benefit against any side effects and such dosages may vary depending upon the therapeutic application for which the recombinant vector is employed. The levels of expression of antigen(s) can be monitored to determine the frequency of dosage administration.

Recombinant, replication defective adenoviruses can be administered in a “pharmaceutically effective amount”, that is, an amount of recombinant adenovirus that is effective in a route of administration to transfect the desired cells and provide sufficient levels of expression of the selected gene to provide a vaccinal benefit, i.e., some measurable level of protective immunity. C68 vectors comprising an antigen cassette can be co-administered with adjuvant. Adjuvant can be separate from the vector (e.g., alum) or encoded within the vector, in particular if the adjuvant is a protein. Adjuvants are well known in the art.

Conventional and pharmaceutically acceptable routes of administration include, but are not limited to, intranasal, intramuscular, intratracheal, subcutaneous, intradermal, rectal, oral and other parental routes of administration. Routes of administration may be combined, if desired, or adjusted depending upon the immunogen or the disease. For example, in prophylaxis of rabies, the subcutaneous, intratracheal and intranasal routes are preferred. The route of administration primarily will depend on the nature of the disease being treated.

The levels of immunity to antigen(s) can be monitored to determine the need, if any, for boosters. Following an assessment of antibody titers in the serum, for example, optional booster immunizations may be desired

### VI. Therapeutic and Manufacturing Methods

Also provided is a method of inducing an infectious disease organism-specific (e.g. a coronavirus specific) immune response in a subject, vaccinating against an infectious disease organism, treating and/or alleviating a symptom of an infection associated with an infectious disease organism in a subject by administering to the subject one or more antigens such as a plurality of antigens identified using methods disclosed herein.

In some aspects, a subject has been diagnosed with an infection or is at risk of an infection (e.g. Covid-19 due to a coronavirus infection), such as age, geographical/travel, and/or work-related increased risk of or predisposition to an infection, or at risk to a seasonal and/or novel disease infection.

In some aspects, a subject is immunocompromised, such as diagnosed with and/or suspected of having cancer. A subject can include those treated with a therapy resulting in immunosuppression. For example, a subject can include those diagnosed with a hematopoietic malignancy and treated with a hematopoietic cell targeting therapy, such as a B cell malignancy treated with an anti-CD20 therapy (e.g., rituximab). In another example, a subject can include those diagnosed with multiple sclerosis [e.g., Relapsing-remitting multiple sclerosis (RRMS), Secondary-progressive multiple sclerosis (SPMS), or Primary-progressive multiple sclerosis (PPMS)] and treated with an anti-CD20 therapy.

An antigen can be administered in an amount sufficient to stimulate a CTL response. An antigen can be administered in an amount sufficient to stimulate a T cell response. An antigen can be administered in an amount sufficient to stimulate a B cell response.

An antigen can be administered alone or in combination with other therapeutic agents. Therapeutic agents can include those that target an infectious disease organism, such as an anti-viral or antibiotic agent.

The optimum amount of each antigen to be included in a vaccine composition and the optimum dosing regimen can be determined. For example, an antigen or its variant can be prepared for intravenous (i.v.) injection, sub-cutaneous (s.c.) injection, intradermal (i.d.) injection, intraperitoneal (i.p.) injection, intramuscular (i.m.) injection. Methods of injection include s.c., i.d., i.p., i.m., and i.v. Methods of DNA or RNA injection include i.d., i.m., s.c., i.p. and i.v. Other methods of administration of the vaccine composition are known to those skilled in the art.

A vaccine can be compiled so that the selection, number and/or amount of antigens present in the composition is/are tissue, infectious disease, and/or patient-specific. For instance, the exact selection of peptides can be guided by expression patterns of the parent proteins in a given tissue or guided by mutation or disease status of a patient. The selection can be dependent on the specific infectious disease (e.g. the specific coronavirus isolate the subject is infected with or at risk for infection by), the status of the disease, the goal of the vaccination (e.g., preventative or targeting an ongoing disease), earlier treatment regimens, the immune status of the patient, and, of course, the HLA-haplotype of the patient. Furthermore, a vaccine can contain individualized components, according to personal needs of the particular patient. Examples include varying the selection of antigens according to the expression of the antigen in the particular patient or adjustments for secondary treatments following a first round or scheme of treatment.

A patient can be identified for administration of an antigen vaccine through the use of various diagnostic methods, e.g., patient selection methods described further below. Patient selection can involve identifying mutations in, or expression patterns of, one or more genes. Patient selection can involve identifying the infectious disease of an ongoing infection (e.g. the presence of a coronavirus infection and/or the specific coronavirus isolate). Patient selection can involve identifying risk of an infection by an infectious disease. In some cases, patient selection involves identifying the haplotype of the patient. The various patient selection methods can be performed in parallel, e.g., a sequencing diagnostic can identify both the mutations and the haplotype of a patient. The various patient selection methods can be performed sequentially, e.g., one diagnostic test identifies the mutations and separate diagnostic test identifies the haplotype of a patient, and where each test can be the same (e.g., both high-throughput sequencing) or different (e.g., one high-throughput sequencing and the other Sanger sequencing) diagnostic methods.

For a composition to be used as a vaccine for an infectious disease, antigens with similar normal self-peptides that are expressed in high amounts in normal tissues can be avoided or be present in low amounts in a composition described herein. On the other hand, if it is known that the infected cell of a patient expresses high amounts of a certain antigen, the respective pharmaceutical composition for treatment of this infection can be present in high amounts and/or more than one antigen specific for this particularly antigen or pathway of this antigen can be included.

Compositions comprising an antigen can be administered to an individual already suffering from an infection. In therapeutic applications, compositions are administered to a patient in an amount sufficient to stimulate an effective CTL response to the infectious disease organism antigen and to cure or at least partially arrest symptoms and/or complications. An amount adequate to accomplish this is defined as “therapeutically effective dose.” Amounts effective for this use will depend on, e.g., the composition, the manner of administration, the stage and severity of the disease being treated, the weight and general state of health of the patient, and the judgment of the prescribing physician. It should be kept in mind that compositions can generally be employed in serious disease states, that is, life-threatening or potentially life threatening situations, especially when the infectious disease organism has induced organ damage and/or other immune pathology. In such cases, in view of the minimization of extraneous substances and the relative nontoxic nature of an antigen, it is possible and can be felt desirable by the treating physician to administer substantial excesses of these compositions.

For therapeutic use, administration can begin at the detection or treatment of an infection. This can be followed by boosting doses until at least symptoms are substantially abated and for a period thereafter.

The pharmaceutical compositions (e.g., vaccine compositions) for therapeutic treatment are intended for parenteral, topical, nasal, oral or local administration. A pharmaceutical compositions can be administered parenterally, e.g., intravenously, subcutaneously, intradermally, or intramuscularly. The compositions can be administered to target specific infected tissues and/or cells of a subject. Disclosed herein are compositions for parenteral administration which comprise a solution of the antigen and vaccine compositions are dissolved or suspended in an acceptable carrier, e.g., an aqueous carrier. A variety of aqueous carriers can be used, e.g., water, buffered water, 0.9% saline, 0.3% glycine, hyaluronic acid and the like. These compositions can be sterilized by conventional, well known sterilization techniques, or can be sterile filtered. The resulting aqueous solutions can be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc.

Antigens can also be administered via liposomes, which target them to a particular cells tissue, such as lymphoid tissue. Liposomes are also useful in increasing half-life. Liposomes include emulsions, foams, micelles, insoluble monolayers, liquid crystals, phospholipid dispersions, lamellar layers and the like. In these preparations the antigen to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired antigen can be directed to the site of lymphoid cells, where the liposomes then deliver the selected therapeutic/immunogenic compositions. Liposomes can be formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al., Ann. Rev. Biophys. Bioeng. 9; 467 (1980), U.S. Pat. Nos. 4,235,871, 4,501,728, 4,501,728, 4,837,028, and 5,019,369.

For targeting to the immune cells, a ligand to be incorporated into the liposome can include, e.g., antibodies or fragments thereof specific for cell surface determinants of the desired immune system cells. A liposome suspension can be administered intravenously, locally, topically, etc. in a dose which varies according to, inter alia, the manner of administration, the peptide being delivered, and the stage of the disease being treated.

For therapeutic or immunization purposes, nucleic acids encoding a peptide and optionally one or more of the peptides described herein can also be administered to the patient. A number of methods are conveniently used to deliver the nucleic acids to the patient. For instance, the nucleic acid can be delivered directly, as “naked DNA”. This approach is described, for instance, in Wolff et al., Science 247: 1465-1468 (1990) as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles. Approaches for delivering nucleic acid sequences can include viral vectors, mRNA vectors, and DNA vectors with or without electroporation.

The nucleic acids can also be delivered complexed to cationic compounds, such as cationic lipids. Lipid-mediated gene delivery methods are described, for instance, in 9618372WOAWO 96/18372; 9324640WOAWO 93/24640; Mannino & Gould-Fogerite, BioTechniques 6(7): 682-691 (1988); U.S. Pat. No. 5,279,833 Rose U.S. Pat. Nos. 5,279,833; 9,106,309WOAWO 91/06309; and Felgner et al., Proc. Natl. Acad. Sci. USA 84: 7413-7414 (1987).

Antigens can also be included in viral vector-based vaccine platforms, such as vaccinia, fowlpox, self-replicating alphavirus, marabavirus, adenovirus (See, e.g., Tatsis et al., Adenoviruses, Molecular Therapy (2004) 10, 616-629), or lentivirus, including but not limited to second, third or hybrid second/third generation lentivirus and recombinant lentivirus of any generation designed to target specific cell types or receptors (See, e.g., Hu et al., Immunization Delivered by Lentiviral Vectors for Cancer and Infectious Diseases, Immunol Rev. (2011) 239(1): 45-61, Sakuma et al., Lentiviral vectors: basic to translational, Biochem J. (2012) 443(3):603-18, Cooper et al., Rescue of splicing-mediated intron loss maximizes expression in lentiviral vectors containing the human ubiquitin C promoter, Nucl. Acids Res. (2015) 43 (1): 682-690, Zufferey et al., Self-Inactivating Lentivirus Vector for Safe and Efficient In Vivo Gene Delivery, J. Virol. (1998) 72 (12): 9873-9880). Dependent on the packaging capacity of the above mentioned viral vector-based vaccine platforms, this approach can deliver one or more nucleotide sequences that encode one or more antigen peptides. The sequences may be flanked by non-mutated sequences, may be separated by linkers or may be preceded with one or more sequences targeting a subcellular compartment (See, e.g., Gros et al., Prospective identification of neoantigen-specific lymphocytes in the peripheral blood of melanoma patients, Nat Med. (2016) 22 (4):433-8, Stronen et al., Targeting of cancer neoantigens with donor-derived T cell receptor repertoires, Science. (2016) 352 (6291): 1337-41, Lu et al., Efficient identification of mutated cancer antigens recognized by T cells associated with durable tumor regressions, Clin Cancer Res. (2014) 20(13):3401-10). Upon introduction into a host, infected cells express the antigens, and thereby stimulate a host immune (e.g., CTL) response against the peptide(s). Vaccinia vectors and methods useful in immunization protocols are described in, e.g., U.S. Pat. No. 4,722,848. Another vector is BCG (Bacille Calmette Guerin). BCG vectors are described in Stover et al. (Nature 351:456-460 (1991)). A wide variety of other vaccine vectors useful for therapeutic administration or immunization of antigens, e.g., Salmonella typhi vectors, and the like will be apparent to those skilled in the art from the description herein.

A means of administering nucleic acids uses minigene constructs encoding one or multiple epitopes. To create a DNA sequence encoding the selected CTL epitopes (minigene) for expression in human cells, the amino acid sequences of the epitopes are reverse translated. A human codon usage table is used to guide the codon choice for each amino acid. These epitope-encoding DNA sequences are directly adjoined, creating a continuous polypeptide sequence. To optimize expression and/or immunogenicity, additional elements can be incorporated into the minigene design. Examples of amino acid sequence that could be reverse translated and included in the minigene sequence include: helper T lymphocyte, epitopes, a leader (signal) sequence, and an endoplasmic reticulum retention signal. In addition, MHC presentation of CTL epitopes can be improved by including synthetic (e.g. poly-alanine) or naturally-occurring flanking sequences adjacent to the CTL epitopes. The minigene sequence is converted to DNA by assembling oligonucleotides that encode the plus and minus strands of the minigene. Overlapping oligonucleotides (30-100 bases long) are synthesized, phosphorylated, purified and annealed under appropriate conditions using well known techniques. The ends of the oligonucleotides are joined using T4 DNA ligase. This synthetic minigene, encoding the CTL epitope polypeptide, can then cloned into a desired expression vector.

Purified plasmid DNA can be prepared for injection using a variety of formulations. The simplest of these is reconstitution of lyophilized DNA in sterile phosphate-buffer saline (PBS). A variety of methods have been described, and new techniques can become available. As noted above, nucleic acids are conveniently formulated with cationic lipids. In addition, glycolipids, fusogenic liposomes, peptides and compounds referred to collectively as protective, interactive, non-condensing (PINC) could also be complexed to purified plasmid DNA to influence variables such as stability, intramuscular dispersion, or trafficking to specific organs or cell types.

Also disclosed is a method of manufacturing a vaccine, comprising performing the steps of a method disclosed herein; and producing a vaccine comprising a plurality of antigens or a subset of the plurality of antigens.

Antigens disclosed herein can be manufactured using methods known in the art. For example, a method of producing an antigen or a vector (e.g., a vector including at least one sequence encoding one or more antigens) disclosed herein can include culturing a host cell under conditions suitable for expressing the antigen or vector wherein the host cell comprises at least one polynucleotide encoding the antigen or vector, and purifying the antigen or vector. Standard purification methods include chromatographic techniques, electrophoretic, immunological, precipitation, dialysis, filtration, concentration, and chromatofocusing techniques.

Host cells can include a Chinese Hamster Ovary (CHO) cell, NS0 cell, yeast, or a HEK293 cell. Host cells can be transformed with one or more polynucleotides comprising at least one nucleic acid sequence that encodes an antigen or vector disclosed herein, optionally wherein the isolated polynucleotide further comprises a promoter sequence operably linked to the at least one nucleic acid sequence that encodes the antigen or vector. In certain embodiments the isolated polynucleotide can be cDNA.

### VII. Antigen Use and Administration

A vaccination protocol can be used to dose a subject with one or more antigens and/or epitopes. A priming vaccine and a boosting vaccine can be used to dose the subject.

The priming vaccine can be based on C68 (e.g., the sequences shown in SEQ ID NO:1 or 2) or srRNA (e.g., the sequences shown in SEQ ID NO:3 or 4) and the boosting vaccine can be based on C68 (e.g., the sequences shown in SEQ ID NO: 1 or 2) or srRNA (e.g., the sequences shown in SEQ ID NO:3 or 4). Each vector typically includes a cassette that includes antigens and/or epitopes. Cassettes can include about 20 epitopes (or antigens from which the epitopes are derived), separated by spacers such as the natural sequence that normally surrounds each epitope or other non-natural spacer sequences such as AAY. Cassettes can also include MHCII antigens/epitopes such a tetanus toxoid antigen and PADRE antigen, which can be considered universal class II antigens. Cassettes can also include a targeting sequence such as a ubiquitin targeting sequence.

A priming vaccine can be injected (e.g., intramuscularly) in a subject. Bilateral injections per dose can be used. For example, one or more injections of ChAdV68 (C68) can be used (e.g., total dose 1×10¹² viral particles); one or more injections of self-amplifying RNA (samRNA or SAM), such as a dose of 3 μg, 10 μg, 30 μg, 100 μg, or 300 μg RNA can be used. A SAM priming dose of 30 μg or less can be used. A SAM priming dose of 10 μg or less can be used. A SAM priming dose of 3 μg or less can be used. For ChAdV68 priming, 1×10¹² or less of viral particles can be administered. For ChAdV68 priming, 3×10¹¹ or less of the viral particles can be administered. For ChAdV68 priming, at least 1×10¹¹ of the viral particles can be administered. For ChAdV68 priming, between 1×10¹¹ and 1×10¹², between 3×10¹¹ and 1×10¹², or between 1×10¹¹ and 3×10¹¹ of the viral particles can be administered. For ChAdV68 priming, 1×10¹¹, 3×10¹¹, or 1×10¹² of the viral particles can be administered. For ChAdV68 priming, the viral particles can be at a concentration of at 5×10¹¹ vp/mL.

A vaccine boost (boosting vaccine) can be injected (e.g., intramuscularly) after prime vaccination. A boosting vaccine can be administered about every 1, 2, 3, 4, 5, 6, 7, 8, 9, or 10 weeks, e.g., every 4 weeks and/or 8 weeks after the prime. Bilateral injections per dose can be used. For example, one or more injections of ChAdV68 (C68) can be used (e.g., total dose 1×10¹² viral particles); one or more injections of self-amplifying RNA (samRNA or SAM), such as a dose of 3 μg, 10 μg, 30 μg, 100 μg, or 300 μg RNA can be used. A SAM boosting dose of 30 μg or less can be used. A SAM boosting dose of 10 μg or less can be used. A SAM boosting dose of 3 μg or less can be used. One or more injections of samRNA at a dose of 30 μg or less can be used. A dose of 30 μg or less can represent the total content of RNA/samRNA administered. A dose of 30 μg or less can represent the total content of RNA/samRNA administered and include only a single distinct samRNA construct. A SAM boost of between 10-30 μg, 10-100 μg, 10-300 μg, 30-100 μg, 30-300 μg, or 100-300 μg RNA can be administered. A SAM boost of between 10-500 μg, 10-1000 μg, 30-500 μg, 30-1000 μg, or 500-1000 μg RNA can be administered. A SAM boost of at least 400 μg, at least 500 μg, at least 600 μg, at least 700 μg, at least 800 μg, at least 900 μg, at least 1000 μg RNA can be administered. A SAM boost of 10 μg, 30 μg, 100 μg, or 300 μg RNA can be administered. A SAM boost of 300 μg RNA can be administered. A SAM boost of 100 μg RNA can be administered. A SAM boost of 30 μg RNA can be administered. A SAM boost of 10 μg RNA can be administered. A SAM boost of 3 μg RNA can be administered. A SAM boost of at least 300 μg RNA can be administered. A SAM boost of at least 100 μg RNA can be administered. A SAM boost of at least 30 μg RNA can be administered. A SAM boost of at least 10 μg RNA can be administered. A SAM boost of at least 3 μg RNA can be administered. A SAM boost of less than or equal to 300 μg RNA can be administered. A SAM boost of less than or equal to 100 μg RNA can be administered.

A dose of can represent the total content of RNA/samRNA administered. A dose of can represent the total content of RNA/samRNA administered and include only a single distinct samRNA construct.

Immune monitoring can be performed before, during, and/or after vaccine administration. Such monitoring can inform safety and efficacy, among other parameters.

To perform immune monitoring, PBMCs are commonly used. PBMCs can be isolated before prime vaccination, and after prime vaccination (e.g. 4 weeks and 8 weeks). PBMCs can be harvested just prior to boost vaccinations and after each boost vaccination (e.g. 4 weeks and 8 weeks).

T cell responses can be assessed as part of an immune monitoring protocol. For example, the ability of a vaccine composition described herein to stimulate an immune response can be monitored and/or assessed. As used herein, “stimulate an immune response” refers to any increase in a immune response, such as initiating an immune response (e.g., a priming vaccine stimulating the initiation of an immune response in a naïve subject) or enhancement of an immune response (e.g., a boosting vaccine stimulating the enhancement of an immune response in a subject having a pre-existing immune response to an antigen, such as a pre-existing immune response initiated by a priming vaccine). Enhancing an immune response can include stimulating an immune response in a convalescent subject (e.g., a boosting vaccine stimulating the enhancement of an immune response in a convalescent Covid-19 subject). A subject can include an HIV positive subject. T cell responses can be measured using one or more methods known in the art such as ELISpot, intracellular cytokine staining, cytokine secretion and cell surface capture, T cell proliferation, MHC multimer staining, or by cytotoxicity assay. T cell responses to epitopes encoded in vaccines can be monitored from PBMCs by measuring induction of cytokines, such as IFN-gamma, using an ELISpot assay. Specific CD4 or CD8 T cell responses to epitopes encoded in vaccines can be monitored from PBMCs by measuring induction of cytokines captured intracellularly or extracellularly, such as IFN-gamma, using flow cytometry. Specific CD4 or CD8 T cell responses to epitopes encoded in the vaccines can be monitored from PBMCs by measuring T cell populations expressing T cell receptors specific for epitope/MHC class I complexes using MHC multimer staining. Specific CD4 or CD8 T cell responses to epitopes encoded in the vaccines can be monitored from PBMCs by measuring the ex vivo expansion of T cell populations following 3H-thymidine, bromodeoxyuridine and carboxyfluoresceine-diacetate-succinimidylester (CFSE) incorporation. The antigen recognition capacity and lytic activity of PBMC-derived T cells that are specific for epitopes encoded in vaccines can be assessed functionally by chromium release assay or alternative colorimetric cytotoxicity assays.

B cell responses can be measured using one or more methods known in the art such as assays used to determine B cell differentiation (e.g., differentiation into plasma cells), B cell or plasma cell proliferation, B cell or plasma cell activation (e.g., upregulation of costimulatory markers such as CD80 or CD86), antibody class switching, and/or antibody production (e.g., an ELISA).

Pancorona vaccination methods can include administering RBD derived nucleic acid sequences that are encoded by a single antigen cassette (e.g., a multi-cistronic cassette). Pancorona vaccination methods can include administering RBD derived nucleic acid sequences that are encoded by separate polynucleotide sequences (e.g., where each RBD is encoded on a separate viral backbone), such as a “blended” vaccine strategy of administering multiple distinct vaccines each encoding a separate distinct RBD alone (e.g., separate administrations of each) or in combination (a single administration of a combination of distinct RBD-encoding delivery vectors). Pancorona vaccination methods can include administering RBD derived nucleic acid sequences, either encoded by a single antigen cassette (e.g., a multi-cistronic cassette) or as multiple distinct vaccines each encoding a separate distinct RBD alone, together with additional antigen-encoding nucleic acid sequences, such as encode a MHC class I epitope, a MHC class II epitope, an epitope capable of stimulating a B cell response, or a combination thereof. In an illustrative non-limiting example, pancorona vaccination methods can include administering a vaccine with RBD derived nucleic acid sequences encoded in a single antigen cassette together with a vaccine with a cassette encoding concatenated T cell epitopes (e.g., see Table 16A-D), either as separate administrations of each or co-formulated together as a combined single administration.

### VIII. Isolation and Detection of HLA Peptides

Isolation of HLA-peptide molecules was performed using classic immunoprecipitation (IP) methods after lysis and solubilization of the tissue sample (55-58). Examples and methods are described in more detail in international patent application publication WO/2018/208856, herein incorporated by reference, in its entirety, for all purposes.

### IX. Presentation Model

Presentation models can be used to identify likelihoods of peptide presentation in patients. Various presentation models are known to those skilled in the art, for example the presentation models described in more detail in U.S. Pat. No. 10,055,540, US Application Pub. No. US20200010849A1 and US20110293637, and international patent application publications WO/2018/195357, WO/2018/208856, and WO2016187508, each herein incorporated by reference, in their entirety, for all purposes.

### X. Training Module

Training modules can be used to construct one or more presentation models based on training data sets that generate likelihoods of whether peptide sequences will be presented by MHC alleles associated with the peptide sequences. Various training modules are known to those skilled in the art, for example the presentation models described in more detail in U.S. Pat. No. 10,055,540, US Application Pub. No. US20200010849A1, and international patent application publications WO/2018/195357 and WO/2018/208856, each herein incorporated by reference, in their entirety, for all purposes. A training module can construct a presentation model to predict presentation likelihoods of peptides on a per-allele basis. A training module can also construct a presentation model to predict presentation likelihoods of peptides in a multiple-allele setting where two or more MHC alleles are present.

### XI. Prediction Module

A prediction module can be used to receive sequence data and select candidate antigens in the sequence data using a presentation model. Specifically, the sequence data may be DNA sequences, RNA sequences, and/or protein sequences extracted from infected cells patients or infectious disease organisms themselves (e.g., coronavirus). A prediction module may identify candidate antigens that are pathogen-derived peptides (e.g., coronavirus derived), such as by comparing sequence data extracted from normal tissue cells of a patient with the sequence data extracted from infected cells of the patient to identify portions containing one or more infectious disease organism associated antigens. A prediction module may identify candidate antigens that are expressed in an infected cell or infected tissue in comparison to a normal cell or tissue by comparing sequence data extracted from normal tissue cells of a patient with the sequence data extracted from infected tissue cells of the patient to identify expressed candidate antigens (e.g., identifying expressed polynucleotides and/or polypeptides specific to an infectious disease).

A presentation module can apply one or more presentation model to processed peptide sequences to estimate presentation likelihoods of the peptide sequences. Specifically, the prediction module may select one or more candidate antigen peptide sequences that are likely to be presented on infected cell HLA molecules by applying presentation models to the candidate antigens. In one implementation, the presentation module selects candidate antigen sequences that have estimated presentation likelihoods above a predetermined threshold. In another implementation, the presentation model selects the N candidate antigen sequences that have the highest estimated presentation likelihoods (where N is generally the maximum number of epitopes that can be delivered in a vaccine). A vaccine including the selected candidate antigens for a given patient can be injected into the patient to stimulate immune responses.

### XI.B.Cassette Design Module

### XI.B.1 Overview

A cassette design module can be used to generate a vaccine cassette sequence based on selected candidate peptides for injection into a patient. For example, a cassette design module can be used to generate a sequence encoding concatenated epitope sequences, such as concatenated T cell epitopes. Various cassette design modules are known to those skilled in the art, for example the cassette design modules described in more detail in U.S. Pat. No. 10,055,540, US Application Pub. No. US20200010849A1, and international patent application publications WO/2018/195357 and WO/2018/208856, each herein incorporated by reference, in their entirety, for all purposes.

A set of therapeutic epitopes may be generated based on the selected peptides determined by a prediction module associated with presentation likelihoods above a predetermined threshold, where the presentation likelihoods are determined by the presentation models. However it is appreciated that in other embodiments, the set of therapeutic epitopes may be generated based on any one or more of a number of methods (alone or in combination), for example, based on binding affinity or predicted binding affinity to HLA class I or class II alleles of the patient, binding stability or predicted binding stability to HLA class I or class II alleles of the patient, random sampling, and the like.

Therapeutic epitopes may correspond to selected peptides themselves. Therapeutic epitopes may also include C- and/or N-terminal flanking sequences in addition to the selected peptides. N- and C-terminal flanking sequences can be the native N- and C-terminal flanking sequences of the therapeutic vaccine epitope in the context of its source protein. Therapeutic epitopes can represent a fixed-length epitope Therapeutic epitopes can represent a variable-length epitope, in which the length of the epitope can be varied depending on, for example, the length of the C- or N-flanking sequence. For example, the C-terminal flanking sequence and the N-terminal flanking sequence can each have varying lengths of 2-5 residues, resulting in 16 possible choices for the epitope.

A cassette design module can also generate cassette sequences by taking into account presentation of junction epitopes that span the junction between a pair of therapeutic epitopes in the cassette. Junction epitopes are novel non-self but irrelevant epitope sequences that arise in the cassette due to the process of concatenating therapeutic epitopes and linker sequences in the cassette. The novel sequences of junction epitopes are different from the therapeutic epitopes of the cassette themselves.

A cassette design module can generate a cassette sequence that reduces the likelihood that junction epitopes are presented in the patient. Specifically, when the cassette is injected into the patient, junction epitopes have the potential to be presented by HLA class I or HLA class II alleles of the patient, and stimulate a CD8 or CD4 T-cell response, respectively. Such reactions are often times undesirable because T-cells reactive to the junction epitopes have no therapeutic benefit, and may diminish the immune response to the selected therapeutic epitopes in the cassette by antigenic competition.⁷⁶

A cassette design module can iterate through one or more candidate cassettes, and determine a cassette sequence for which a presentation score of junction epitopes associated with that cassette sequence is below a numerical threshold. The junction epitope presentation score is a quantity associated with presentation likelihoods of the junction epitopes in the cassette, and a higher value of the junction epitope presentation score indicates a higher likelihood that junction epitopes of the cassette will be presented by HLA class I or HLA class II or both.

In one embodiment, a cassette design module may determine a cassette sequence associated with the lowest junction epitope presentation score among the candidate cassette sequences.

A cassette design module may iterate through one or more candidate cassette sequences, determine the junction epitope presentation score for the candidate cassettes, and identify an optimal cassette sequence associated with a junction epitope presentation score below the threshold.

A cassette design module may further check the one or more candidate cassette sequences to identify if any of the junction epitopes in the candidate cassette sequences are self-epitopes for a given patient for whom the vaccine is being designed. To accomplish this, the cassette design module checks the junction epitopes against a known database such as BLAST. In one embodiment, the cassette design module may be configured to design cassettes that avoid junction self-epitopes.

A cassette design module can perform a brute force approach and iterate through all or most possible candidate cassette sequences to select the sequence with the smallest junction epitope presentation score. However, the number of such candidate cassettes can be prohibitively large as the capacity of the vaccine increases. For example, for a vaccine capacity of 20 epitopes, the cassette design module has to iterate through ˜10¹⁸ possible candidate cassettes to determine the cassette with the lowest junction epitope presentation score. This determination may be computationally burdensome (in terms of computational processing resources required), and sometimes intractable, for the cassette design module to complete within a reasonable amount of time to generate the vaccine for the patient. Moreover, accounting for the possible junction epitopes for each candidate cassette can be even more burdensome. Thus, a cassette design module may select a cassette sequence based on ways of iterating through a number of candidate cassette sequences that are significantly smaller than the number of candidate cassette sequences for the brute force approach.

A cassette design module can generate a subset of randomly or at least pseudo-randomly generated candidate cassettes, and selects the candidate cassette associated with a junction epitope presentation score below a predetermined threshold as the cassette sequence. Additionally, the cassette design module may select the candidate cassette from the subset with the lowest junction epitope presentation score as the cassette sequence. For example, the cassette design module may generate a subset of ˜1 million candidate cassettes for a set of 20 selected epitopes, and select the candidate cassette with the smallest junction epitope presentation score. Although generating a subset of random cassette sequences and selecting a cassette sequence with a low junction epitope presentation score out of the subset may be sub-optimal relative to the brute force approach, it requires significantly less computational resources thereby making its implementation technically feasible. Further, performing the brute force method as opposed to this more efficient technique may only result in a minor or even negligible improvement in junction epitope presentation score, thus making it not worthwhile from a resource allocation perspective. A cassette design module can determine an improved cassette configuration by formulating the epitope sequence for the cassette as an asymmetric traveling salesman problem (TSP). Given a list of nodes and distances between each pair of nodes, the TSP determines a sequence of nodes associated with the shortest total distance to visit each node exactly once and return to the original node. For example, given cities A, B, and C with known distances between each other, the solution of the TSP generates a closed sequence of cities, for which the total distance traveled to visit each city exactly once is the smallest among possible routes. The asymmetric version of the TSP determines the optimal sequence of nodes when the distance between a pair of nodes are asymmetric. For example, the “distance” for traveling from node A to node B may be different from the “distance” for traveling from node B to node A. By solving for an improved optimal cassette using an asymmetric TSP, the cassette design module can find a cassette sequence that results in a reduced presentation score across the junctions between epitopes of the cassette. The solution of the asymmetric TSP indicates a sequence of therapeutic epitopes that correspond to the order in which the epitopes should be concatenated in a cassette to minimize the junction epitope presentation score across the junctions of the cassette. A cassette sequence determined through this approach can result in a sequence with significantly less presentation of junction epitopes while potentially requiring significantly less computational resources than the random sampling approach, especially when the number of generated candidate cassette sequences is large. Illustrative examples of different computational approaches and comparisons for optimizing cassette design are described in more detail in U.S. Pat. No. 10,055,540, US Application Pub. No. US20200010849A1, and international patent application publications WO/2018/195357 and WO/2018/208856, each herein incorporated by reference, in their entirety, for all purposes.

A cassette design module can also generate cassette sequences by taking into account additional protein sequences encoded in the vaccine. For example, a cassette design module used to generate a sequence encoding concatenated T cell epitopes can take into account T cell epitopes already encoded by additional protein sequences present in the vaccine (e.g., full-length protein sequences), such as by removing T cell epitopes already encoded by the additional protein sequences from the list of candidate sequences.

A cassette design module can also generate cassette sequences by taking into account the size of the sequences. Without wishing to be bound by theory, in general, increased cassette size can negatively impact vaccine aspects, such as vaccine production and/or vaccine efficacy. In one example, the cassette design module can take into account overlapping sequences, such as overlapping T cell epitope sequences. In general, a single sequence containing overlapping T cell epitope sequences (also referred to as a “frame”) is more efficient than separately linking individual T cell epitope sequences as it reduces the sequence size needed to encode the multiple peptides. Accordingly, in an illustrative example, a cassette design module used to generate a sequence encoding concatenated T cell epitopes can take into account the cost/benefit of extending a candidate T cell epitope to encode one or more additional T cell epitopes, such as determining the benefit gained in additional population coverage for an MHC presenting the additional T cell epitope versus the cost of increasing the size of the sequence.

A cassette design module can also generate cassette sequences by taking into account the magnitude of stimulation of an immune response generated by validated epitopes.

A cassette design module can also generate cassette sequences by taking into account presentation of encoded epitopes across a population, for example that at least one immunogenic epitope is presented by at least one HLA across a proportion of a population, for example by at least 85%, 90%, or 95% of a population (e.g., HLA-A, HLA-B and HLA-C genes over four major ethnic groups, namely European (EUR), African American (AFA), Asian and Pacific Islander (APA) and Hispanic (HIS)). As an illustrative non-limiting example, a cassette design module can also generate cassette sequences such that at least one HLA is present at least across 85%, 90%, or 95% of a population that presents at least one validated epitope or presents at least 4, 5, 6, or 7 predicted epitopes.

A cassette design module can also generate cassette sequences by taking into account other aspects that improve potential safety, such as limiting encoding or the potential to encode a functional protein, functional protein domain, functional protein subunit, or functional protein fragment potentially presenting a safety risk. In some cases, a cassette design module can limit sequence size of encoded peptides such that are less than 50%, less than 49%, less than 48%, less than 47%, less than 46%, less than 45%, less than 45%, less than 43%, less than 42%, less than 41%, less than 40%, less than 39%, less than 38%, less than 37%, less than 36%, less than 35%, less than 34%, or less than 33% of the translated, corresponding full-length protein. In some cases, a cassette design module can limit sequence size of encoded peptides such that a single contiguous sequence is less than 50% of the translated, corresponding full-length protein, but more than one sequence may be derived from the same translated, corresponding full-length protein and together encode more than 50%. In an illustrative example, if a single sequence containing overlapping T cell epitope sequences (“frame”) is larger than 50% of the translated, corresponding full-length protein, the frame can be split into multiple frames (e.g., f1, f2 etc.) such that each frame is less than 50% of the translated, corresponding full-length protein. A cassette design module can also limit sequence size of encoded peptides such that a single contiguous sequence is less than 49%, less than 48%, less than 47%, less than 46%, less than 45%, less than 45%, less than 43%, less than 42%, less than 41%, less than 40%, less than 39%, less than 38%, less than 37%, less than 36%, less than 35%, less than 34%, or less than 33% of the translated, corresponding full-length protein. Where multiple frames from the same gene are encoded, the multiple frames can have overlapping sequences with each other, in other words each separately encode the same sequence. Where multiple frames from the same gene are encoded, the two or more nucleic acid sequences derived from the same gene can be ordered such that a first nucleic acid sequence cannot be immediately followed by or linked to a second nucleic acid sequence if the second nucleic acid sequence follows, immediately or not, the first nucleic acid sequence in the corresponding gene. For example, if there are 3 frames within the same gene (f1,f2,f3 in increasing order of amino acid position):
The following cassette orderings are not allowed:
f1 immediately followed by f2
f2 immediately followed by f3
f1 immediately followed by f3
The following cassette orderings are allowed:
f3 immediately followed by f2
f2 immediately followed by f1

### XIII. Example Computer

A computer can be used for any of the computational methods described herein. One skilled in the art will recognize a computer can have different architectures. Examples of computers are known to those skilled in the art, for example the computers described in more detail in U.S. Pat. No. 10,055,540, US Application Pub. No. US20200010849A1, and international patent application publications WO/2018/195357 and WO/2018/208856, each herein incorporated by reference, in their entirety, for all purposes.

### XIV. Examples

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B(1992).

### XIV.A. SARS-COV-2 MHC Epitope Prediction and Vaccine Cassette Construction

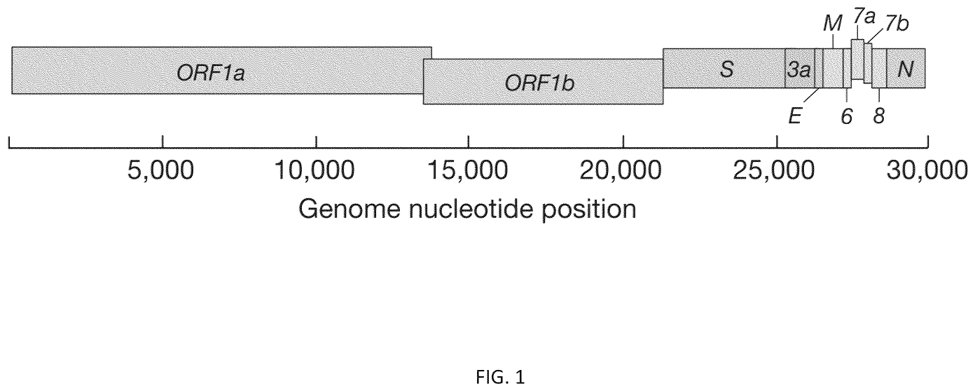

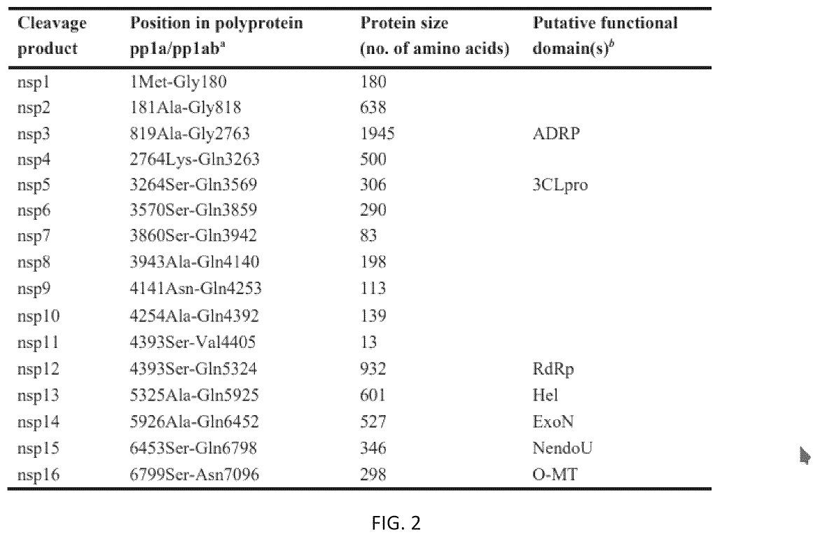

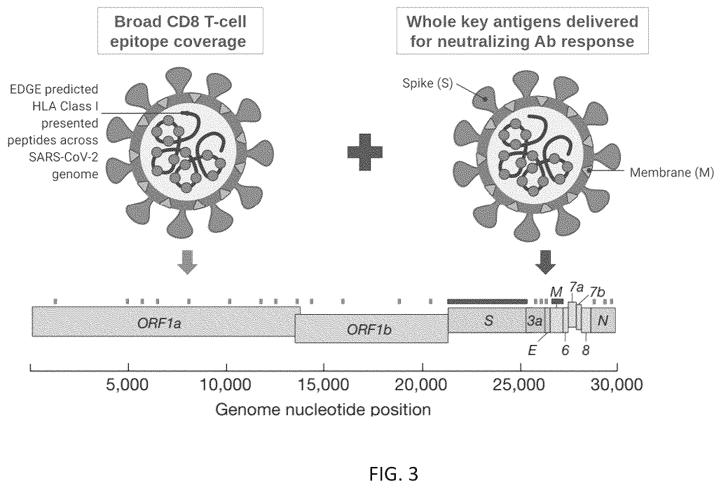

The SARS-COV-2 belongs to the coronavirus family and its reference genome is a single-stranded RNA sequence of 29,903 base pairs. The genome contains at least 14 open reading frames (ORF) as shown in FIG. 1. Among the encoded genes, the essential genes are replicase ORF1ab, spike (S), envelope (E), membrane (M) and nucleocapsid (N). The replicase ORF1ab (position 266-21555) encode two proteins namely orf1a and orf1b, the latter is translated by a ribosomal frameshift by -1 at position 13468. The two proteins together contain 16 non-structure proteins (nsp1-nsp16), as depicted in FIG. 2, that is, the ORF1a and ORF1b are cleaved into 16 nsps. The spike protein is thought to bind to the ACE2 receptor of the human cell, allowing the virus to enter the human cell to use its replication machinery to produce and disseminate more copies of the virus.

Because RNA viruses are known to have high mutation rates, a large number of SARS-COV-2 genomes were analyzed to identify regions in the proteome that are variable. Over 8000 SARS-COV-2 complete genomes deposited into the GISAID database [www.gisaid.org] as of Apr. 19, 2020 were obtained. Pairwise global alignment of each of the genomes to the SARS-COV-2 reference genome (Genbank Accession number NC_045512; SEQ ID NO:76) was performed. Sequences on these genomes that are aligned to coding regions of the reference genome were specifically located the. These sequences were then translated to obtain the protein sequences of these SARS-COV-2. These protein sequences wee then aligned to the respective reference protein sequences to identify variants.

The analysis identified 20 sites on the protein sequences that have a variant rate greater than 1%. These sites are shown in Table 1. In selecting T-cell epitopes, candidate epitopes that cross these variable sites were excluded.

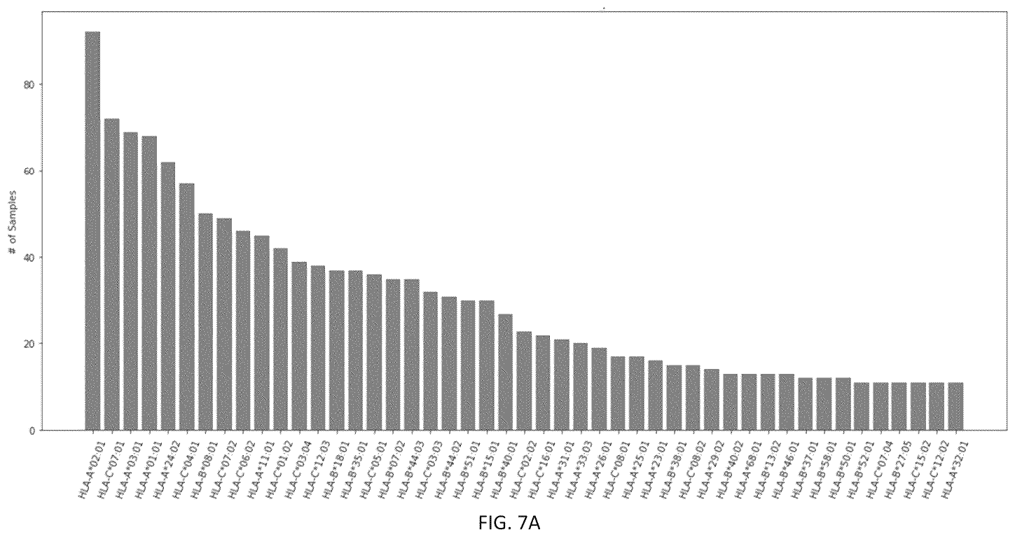

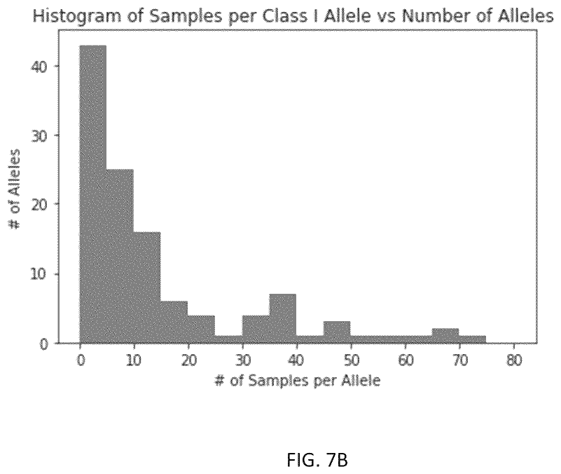

CD8+epitopes were predicted using our machine learning EDGE platform (see U.S. Pat. No. 10,055,540, herein incorporated by reference for all purposes), which was shown to be best-in-class [Bulik-Sullivan et al. (2018). Deep learning using tumor HLA peptide mass spectrometry datasets improves neoantigen identification. Nature Biotechnology 2018, 37(1), herein incorporated by reference for all purposes]. The model for predicting class I epitopes was recently trained on 507,502 peptides presented in Mass Spectrometry across 398 samples and covers 116 identified alleles, of which 112 alleles (Table 2, FIG. 7) are represented in the haplotype distribution dataset described below.

In order to generate a list of candidate CD8+ T-cell epitopes, the orf1ab protein was split at the cleavage sites shown in FIG. 2. Studies show that the spike protein harbors a furin-like cleavage motif at position 681-684, where the cleavage event occurs following position 684 [Wrapp et al. (2020). Cryo-EM structure of the 2019-nCOV spike in the prefusion conformation. Science, 367(6483), 1260-1263, Ou et al. (2020). Characterization of spike glycoprotein of SARS-COV-2 on virus entry and its immune cross-reactivity with SARS-COV. Nature Communications, 11(1), 1620]. The cleavage of the spike protein into SI and S2 is thought to facilitate the cell entry contributing to the transmissibility of the virus. Accordingly, the Spike protein was split at the furin cleavage site for generating candidate CD8+ T-cell epitopes. All 8-11mer peptides were then generated from the cleaved proteins and the other proteins, flanked by their native N-terminal and C-terminal 5-mers.

The EDGE machine learning model was run on these candidate epitopes for each HLA class I allele. That is, the presentation score of a candidate epitope is given an EDGE score for each HLA allele. Generally, the probability of a peptide being presented is influenced by the family of the protein containing the peptide, and the expression level of the protein. The EDGE model was also trained on human peptidome datasets. Given there is no equivalent protein family for SARS-COV-2, for predicting the presentation of a given Sar-COV-2 peptide, a random protein family was assigned to all peptides. Assigning the same protein family, albeit random, will have the same effect on all SARS-COV-2 peptides. A high level of expression was also used (tpm=10). A list of candidate epitopes with the EDGE score of 0.001 and above for an HLA allele are shown in Table A, as well as cognate HLA alleles with a predicted EDGE score greater than 0.001, with each cognate pairing ranked as H (EDGE score >0.1), M (EDGE score between 0.01 and 0.1), and L (EDGE score <0.01).

In order to account for the different levels of expression of SARS-COV-2 genes, the ratio of reported T-cell responses among genes from SARS-COV-2 genome [Li et al. (2008) T Cell Responses to Whole SARS Coronavirus in Humans. The Journal of Immunology, 181(8), 5490-5500] was used as a proxy for the ratio of gene expression levels. The score of all epitopes from a SARS-COV-2 gene was then scaled so that the ratio between the 99^{th} percentile of the epitopes in the selected gene and the 99^{th} percentile of the epitopes in the Spike gene followed the ratio reported in [Li et al. (2008). T Cell Responses to Whole SARS Coronavirus in Humans. The Journal of Immunology, 181(8), 5490-5500].

A set of candidate CD8+epitopes was then selected by choosing those with the scaled EDGE score greater than or equal to a threshold of t=0.01. The threshold was selected from analysis of an HIV LANL dataset (data not shown) so that PPV for T-cell epitopes estimated to be 0.2 and recall is 0.5. The set sequences that are >=90% homologous to known SARs-COV T-cell epitopes reported in IEDB [Vita et al. (2019). The Immune Epitope Database (IEDB): 2018 update. Nucleic Acids Research, 47(D1), D339-D343.] was also included similar to the approach described in Grifoni et al. [(2020). A Sequence Homology and Bioinformatic Approach Can Predict Candidate Targets for Immune Responses to SARS-COV-2. Cell Host & Microbe, 27(4), 671-680.c2].

The set of candidate epitopes excluded those sequences that contained at least one of the sites that have a variable rate greater than 0.01, as mentioned above and shown in Table 1.

In order to maximize the coverage of the vaccine over the world population, allele frequencies of HLA-A, HLA-B and HLA-C genes over four major ethnic groups, namely European (EUR), African American (AFA), Asian and Pacific Islander (APA) and Hispanic (HIS) were obtained from the publicly available National Marrow Donor Program dataset [bioinformatics.bethematchclinical.org/hla-resources/haplotype-frequencies/high-resolution-hla-alleles-and-haplotypes-in-the-us-population]. Simulations were then performed to estimate the frequencies of the haplotypes made up by combination of these HLA alleles.

Cassette optimization proceeded as follows:

### Epitope Selection Definitions

Candidate epitope set E: set of candidate CD8+epitopes with scaled EDGE score greater than or equal to a threshold of t=0.01
Population coverage criteria P: For each of the four ethnicity groups as described above (EUR/AFA/APA/HIS), 95% of the simulated people in that ethnic group have at least 30 candidate epitopes presented in their diplotype
Solution frame set F—all amino acid ranges in the current solution encapsulating the added candidate epitopes
   For each epitope added to the solution, the epitope and 5 flanking native amino acids on each end must be fully contained in a frame of F
   Each frame spans only protein region (including individual NSPs in orf1ab)

### Epitope Selection Method

Population coverage criteria P starts as calculated with all epitopes in the whole gene(s) initially added
If the population coverage criteria P is not satisfied, continually choose the amino acid frame f across SARS-COV-2 proteome that most efficiently maximizes progress to P
   Defined as the highest ratio of additional population coverage C/additional amino acid bases added AA. (C/AA).
   All candidate frames are either a new 25aa frame (AA=25), or can overlap with frames in the existing solution F—in which case it can add any amount less than 25aa (AA<25)
   Additional population coverage C is the increase in epitope count from E for haplotypes with <20 covered epitopes, weighted(multiplied) by the haplotype's population frequency summed across all four ethnic groups
   20 epitopes per haplotype is determined (experimentally chosen) to be an efficient proxy towards reaching the overall coverage criteria of 30 candidate epitopes per diplotype
   Add f to solution frame set F. Remove from E, candidate epitopes within f.
After frames are chosen, create the final set F:
   First merge overlapping frames, yielding contiguous sequences (i.e. epitope “hotspots”)
   Next ensure all frames are less than 50% of that frame's overall gene size. If frames are larger than this size, divide them into smaller (potentially overlapping) frames that are each smaller than this requirement. Additional more stringent size limitations can be tested
To illustrates the marginal value of larger cassette sizes, frame selection can continue past when P is satisfied—but does not affect the composition of the chosen cassette for the criteria P.

### Cassette Ordering

The frames in solution frame set F are ordered to minimize the EDGE score of junction epitopes (unintended epitopes not part of the solution, created by adjacent frames). Successive frames within a gene are also forbidden to immediately follow each other in the cassette (intra-gene restriction). In other words, intra-gene restriction requires that if there are two or more SARS-COV-2 derived nucleic acid sequences encoding epitopes derived from the same SARS-COV-2 gene, the two sequences are ordered such that a first nucleic acid sequence cannot be immediately followed by or linked to a second nucleic acid sequence if the second nucleic acid sequence follows first nucleic acid sequence in the corresponding SARS-COV-2 gene. For example, if there are 3 frames within the same gene (f1,f2,f3 in increasing order of amino acid position)
The following cassette orderings are impossible:
   f1 immediately followed by f2
   f2 immediately followed by f3
   f1 immediately followed by f3
The following cassette orderings are possible:
   f3 immediately followed by f2
   f2 immediately followed by f1

### Cassette Ordering Method

Google optimization routing tools [developers.google.com/optimization/routing] are used to perform a traveling salesman optimization route, where the distance between each pair of frames in F is:
Infinite, if the frames are not allowed to follow each other in this order, according to the above intra-gene restriction
Otherwise the sum of junction epitope EDGE scores across all alleles, weighted by allele frequency in population

Route finding of the minimal path distance yields the optimal ordering of the frames in the cassette to minimize junctional epitopes and avoid successive frames within a gene.

### Results

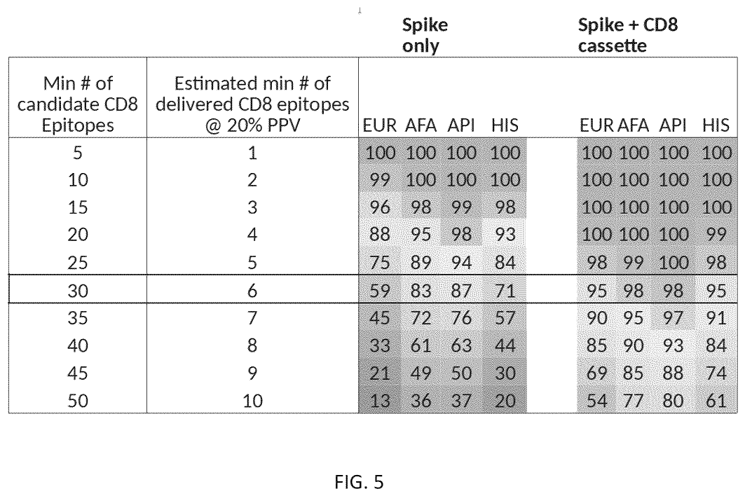

The population coverage criteria P was calculated with all initial epitopes provided by the SARS-COV-2 Spike protein (SEQ ID NO:59) split into SI and S2. Applying the optimization algorithms above yielded a 594 amino acid cassette sequence having 18 epitope-encoding frames, as shown in Table 3A. Table C presents each of the additional epitopes contained in the cassette (not including the epitopes derived from the Spike protein). Empirically, the optimal frame set F was produced when the size threshold for all frames was set to less than 42% of that frame's overall gene size. The coverage of the designed cassette over four populations is shown in FIG. 5, with the first column providing the number of SARS-COV-2 epitopes predicted to be presented and the second column providing the expected number of presented epitopes, based on a 0.2 PPV. Each row shows the protection coverage of each population if a certain number of epitopes is used.

Potential HLA-DRB, HLA-DQ, and HLA-DP MHC class II epitopes from the SARS-COV-2 proteome were also predicted. The method described for generating candidate CD8/MHC class I epitopes was used to generate peptides with sizes between 9 and 20 amino acids. EDGE model was run for class II to compute EDGE score of each of these peptides against each identifiable allele (see, e.g., U.S. application Ser. No. 16/606,577 and international patent application PCT/US2020/021508, each herein incorporated by reference in their entirety for all purposes). The list of CD4 epitopes with EDGE score greater than 0.001 are presented in Table B, as well as cognate HLA alleles with a predicted EDGE score greater than 0.001, with each cognate pairing ranked as H (EDGE score >0.1), M (EDGE score between 0.01 and 0.1), and L (EDGE score <0.01). HLA-DQ and HLA-DP are referred to by their alpha and beta chains used in the analysis, while HLA-DR is referred to by its beta chain as the alpha chain is generally invariable in the human population, with HLA-DR peptide contact regions particularly invariant.

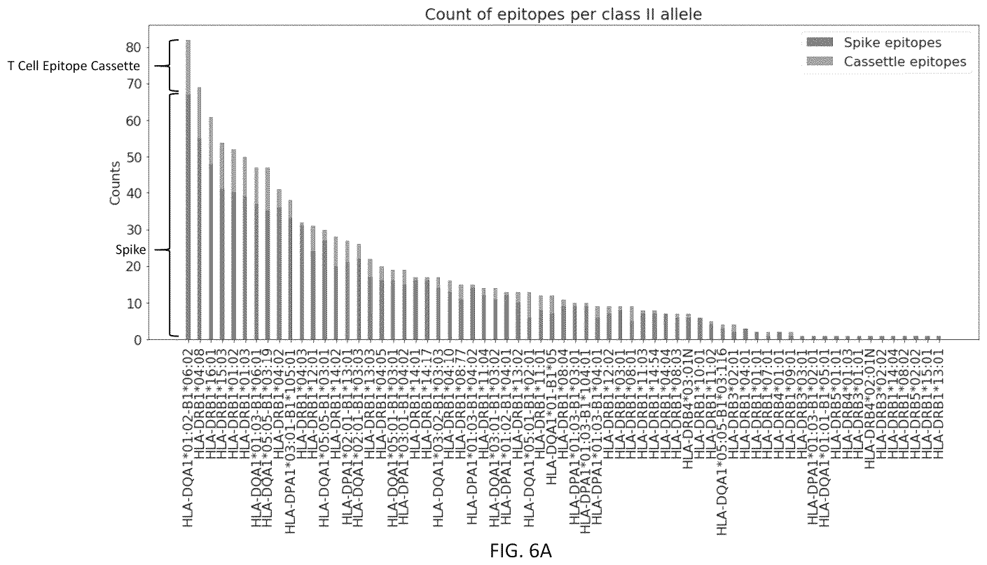

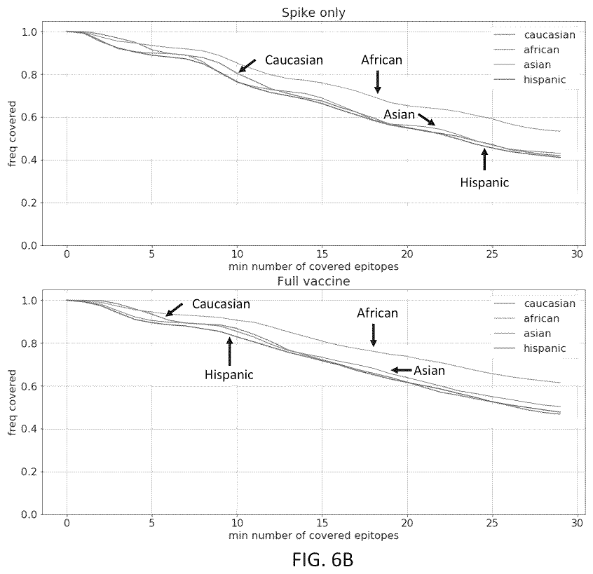

The peptides receiving a score of >0.02 contained in the optimized MHC I cassette frames determined above were then identified. The threshold of 0.02 was chosen because the model prediction has the PPV of 0.2 in predicting Mass Spectrometry data with prevalence ratio positive vs negatives of 1:100. FIG. 6A illustrates the number of predicted epitopes presented by each MHC class II allele examined. FIG. 6B shows the population coverage of MHC class II at the diploid level.

Additional cassettes are designed using the epitope prediction and frame ordering algorithms described above where the initial population coverage criteria P is calculated with all initial epitopes provided by SARS-COV-2 Membrane (SEQ ID NO:61), SARS-COV-2 Nucleocapsid (SEQ ID NO:62). SARS-COV-2 Envelope (SEQ ID NO:63), or combinations (including combinations with SARS-COV-2 spike) or sequence variants thereof.

TABLE 1
Identified SARS-CoV-2 Mutations (>1%)

| | | Reference | Alternate | Variable |
|---|---|---|---|---|
| Gene | Position | Amino Acid | Amino Acid | Rate |
| orf1ab | 265 | T | I | 0.12474 |
| orf1ab | 378 | V | I | 0.022349 |
| orf1ab | 392 | G | D | 0.015073 |
| orf1ab | 448 | D | deletion | 0.030146 |
| orf1ab | 739 | I | V | 0.011435 |
| orf1ab | 765 | P | S | 0.012474 |
| orf1ab | 876 | A | T | 0.015073 |
| orf1ab | 3353 | K | R | 0.023909 |
| orf1ab | 3606 | L | F | 0.130977 |
| orf1ab | 4715 | P | L | 0.448025 |
| orf1ab | 5828 | P | L | 0.193867 |
| orf1ab | 5865 | Y | C | 0.199584 |
| S | 614 | D | G | 0.439815 |
| ORF3a | 57 | Q | H | 0.150943 |
| ORF3a | 251 | G | V | 0.086003 |
| M | 3 | D | G | 0.013508 |
| M | 175 | T | M | 0.051416 |
| ORF8 | 84 | L | S | 0.267563 |
| N | 203 | R | K | 0.124507 |
| N | 204 | G | R | 0.124068 |

TABLE 2
List of Identifiable Class I alleles

HLA-A*01:01
HLA-A*02:01
HLA-A*02:02
HLA-A*02:03
HLA-A*02:04
HLA-A*02:05
HLA-A*02:06
HLA-A*02:07
HLA-A*02:11
HLA-A*03:01
HLA-A*03:02
HLA-A*11:01
HLA-A*11:02
HLA-A*23:01
HLA-A*24:02
HLA-A*24:07
HLA-A*25:01
HLA-A*26:01
HLA-A*26:02
HLA-A*26:03
HLA-A*29:01
HLA-A*29:02
HLA-A*30:01
HLA-A*30:02
HLA-A*31:01
HLA-A*32:01
HLA-A*33:01
HLA-A*33:03
HLA-A*34:01
HLA-A*34:02
HLA-A*36:01
HLA-A*66:01
HLA-A*68:01
HLA-A*68:02
HLA-A*74:01
HLA-B*07:02
HLA-B*07:04
HLA-B*07:05
HLA-B*08:01
HLA-B*13:01
HLA-B*13:02
HLA-B*14:01
HLA-B*14:02
HLA-B*15:01
HLA-B*15:02
HLA-B*15:03
HLA-B*15:10
HLA-B*15:11
HLA-B*15:13
HLA-B*18:01
HLA-B*27:02
HLA-B*27:05
HLA-B*35:01
HLA-B*35:02
HLA-B*35:03
HLA-B*35:08
HLA-B*35:12
HLA-B*37:01
HLA-B*38:01
HLA-B*38:02
HLA-B*39:01
HLA-B*39:06
HLA-B*40:01
HLA-B*40:02
HLA-B*40:06
HLA-B*41:02
HLA-B*42:01
HLA-B*44:02
HLA-B*44:03
HLA-B*44:05
HLA-B*45:01
HLA-B*46:01
HLA-B*48:01
HLA-B*49:01
HLA-B*50:01
HLA-B*51:01
HLA-B*52:01
HLA-B*53:01
HLA-B*54:01
HLA-B*55:01
HLA-B*55:02
HLA-B*56:01
HLA-B*57:01
HLA-B*57:03
HLA-B*58:01
HLA-B*58:02
HLA-C*01:02
HLA-C*02:02
HLA-C*03:02
HLA-C*03:03
HLA-C*03:04
HLA-C*04:01
HLA-C*04:03
HLA-C*05:01
HLA-C*06:02
HLA-C*07:01
HLA-C*07:02
HLA-C*07:04
HLA-C*07:27
HLA-C*08:01
HLA-C*08:02
HLA-C*08:03
HLA-C*12:02
HLA-C*12:03
HLA-C*12:05
HLA-C*14:02
HLA-C*14:03
HLA-C*15:02
HLA-C*15:05
HLA-C*16:01
HLA-C*16:04
HLA-C*17:01

TABLE 3A
Cassette Epitope Frames in Conjunction with Spike Protein

| | | | | Amino Acid |
|---|---|---|---|---|
| Frame | Gene | Gene Start | Gene End | Length |
| 1 | M | 172 | 204 | 33 |
| 2 | orf1ab | 4154 | 4178 | 25 |
| 3 | N | 301 | 345 | 45 |
| 4 | N | 151 | 175 | 25 |
| 5 | N | 71 | 95 | 25 |
| 6 | ORF3a | 106 | 130 | 25 |
| 7 | orf1ab | 4419 | 4443 | 25 |
| 8 | N | 259 | 283 | 25 |
| 9 | orf1ab | 5371 | 5395 | 25 |
| 10 | M | 85 | 140 | 56 |
| 11 | N | 352 | 393 | 42 |
| 12 | orf1ab | 2580 | 2604 | 25 |
| 13 | ORF3a | 1 | 47 | 47 |
| 14 | M | 34 | 60 | 27 |
| 15 | ORF3a | 53 | 86 | 34 |
| 16 | orf1ab | 2794 | 2818 | 25 |
| 17 | ORF3a | 199 | 255 | 57 |
| 18 | E | 44 | 71 | 28 |

### XIV.B. EDGE-Directed Vaccine Design

A series of vaccines against SARS-COV-2 were designed to produce a balanced immune response inducing both neutralizing antibodies (from B cells) as well as effector and memory CD8+ T cell responses for maximum efficacy. In general, neutralizing antibodies to viral surface proteins can serve to prevent viral entry into cells and virus epitope-specific CD8+ T cells kill virally-infected cells. In addition, vaccines against SARS-COV-2 were designed to maximize the coverage of the vaccine across the world population, i.e., target most individuals (e.g., >95%) receive a large number (e.g., >=30) of candidate CD8+epitopes across all major ancestry groups while minimizing our cassette sequence footprint.

### Antigens and Cassettes

Vaccines are constructed encoding the MHC epitope-encoding cassettes designed using the epitope prediction and frame ordering algorithms described above. An exemplary cassette (herein referred to as the Concatenated EDGE predicted SARS-COV-2 MHC Class I Epitope Cassette or EDGE Predicted Epitopes (EPE)) was generated where the initial population coverage criteria P was calculated with all initial epitopes provided by SARS-COV-2 Spike, as described above.

Vaccines are also designed encoding various full-length proteins, either alone or in combination, generally for the purposes of stimulating a B cell response. Full-length proteins include SARS-COV-2 Spike (SEQ ID NO:59), SARS-COV-2 Membrane (SEQ ID NO:61), SARS-COV-2 Nucleocapsid (SEQ ID NO:62), and SARS-COV-2 Envelope (SEQ ID NO:63), sequences of which are shown in Table 3B.

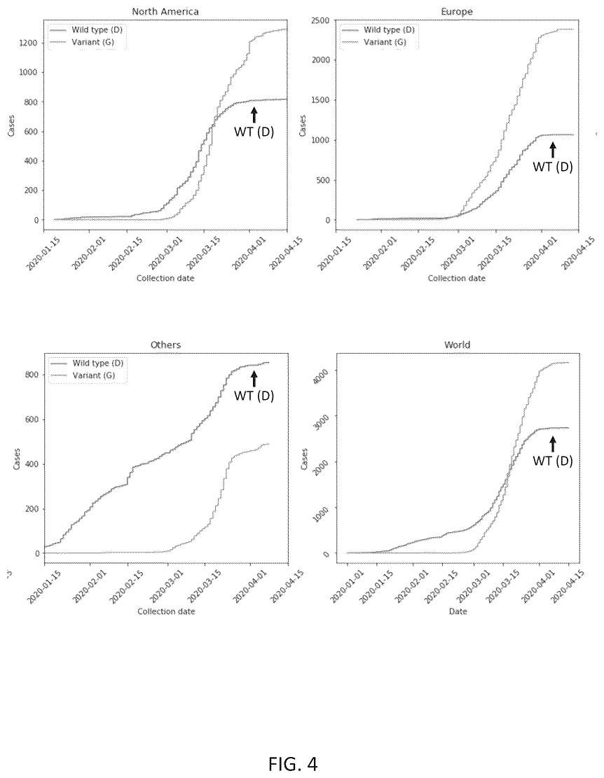

With respect to the Spike protein, initial analysis of prevalent SARS-COV-2 variants (as described above, see Table 1) identified a Spike protein variant present in almost 44% of genomes. Subsequent analysis of the over 8000 SARS-COV-2 complete genomes identified a dominant variant at position 614 where the wildtype amino acid aspartic (D) is mutated to glycine (G). The mutation, denoted as D614G, is found on 60.05% of genomes sequenced worldwide, and 70.46% and 58.49% of the sequences in Europe and North America, respectively (FIG. 4). Accordingly, Spike proteins are used that contain the prevalent D614G Spike variant, with reference to the reference Spike protein (SEQ ID NO:59). In addition, a modified Spike protein was engineered to bias the Spike protein to remain in a predominantly prefusion state, as the prefusion Spike state may be a better target for antibody-mediated neutralization of the virus. The following mutations were selected: R682V to disrupt the Furin cleavage site; R815N to disrupt cleavage site within S2, and K986P and V987P to interfere with the secondary structure of Spike. Accordingly, “modified” Spike proteins are used that contain one or more of the following mutations, with reference to the reference Spike protein (SEQ ID NO:59): a D614G mutation, a R682V mutation, a R815N mutation, a K986P mutation, or a V987P mutation. For reference, a modified Spike proving having all of the Spike mutations is shown in SEQ ID NO:60.

Various vaccine designs and their respective cassette nucleotide sequences are presented in more detail in Table 4. For SAM based vaccines, promoter and/or poly(A) signal sequences can be removed given cassettes are generally operably linked to the endogenous 26S promoter and poly(A) sequence provided by the vector backbone. Depending on the cassette features and configuration, translated proteins (e.g., those in Table 3B) may also include an additional sequence(s) related to the particular expression strategy, such as a 2A ribosome skipping sequence elements (or fragments thereof following translation) and additional 26S promoter sequences.

TABLE 3B
SARS-CoV-2 Proteins

| | | SEQ ID |
|---|---|---|
| Peptide | Amino Acid Sequence | NO: |
| Concatenated | TSRTLSYYKLGASQRVAGDSGFAAYSRYRIGNYCAAGTTQTACTDDN | 57 |
| EDGE predicted | ALAYYNTTKGGWPQIAQFAPSASAFFGMSRIGMEVTPSGTWLTYTGA | |
| SARS-CoV-2 | IKLDDKDPNPANNAAIVLQLPQGTTLPKGFYAEGGVPINTNSSPDDQI | |
| MHC Class I | GYYRRATRRIRLYLYALVYFLQSINFVRIIMRLWLCSTDVVYRAFDIY | |
| Epitope Cassette | NDKVAGFAKFLKTRQKRTATKAYNVTQAFGRRGPEQTQPYVCNAPG | |
| | CDVTDVTQLYLGGMSYYACLVGLMWLSYFIASFRLFARTRSMWSEN | |
| | PETNILLNVPLHGTILTRPLLESELVILLNKHIDAYKTFPPTEPKKDKKK | |
| | KADETQALPQRQKKQQTVTVGDSAEVAVKMFDAYVNTFSSTFNVM | |
| | DLFMRIFTIGTVTLKQGEIKDATPSDFVRATATIPIQASLPFGWLILLQF | |
| | AYANRNRFLYIIKLIFLWLLWPVLAVFQSASKIITLKKRWQLALSKGV | |
| | HFVCNLLLLHVMSKHTDFSSEIIGYKAIDGGVTRDCVVLHSYFTSDYY | |
| | QLYSTQLSTDTGVEHVTFFIYNKIVDEPEEHVQIHTIDGSSGVCNIVNV | |
| | SLVKPSFYVYSRVKNLNSSRVP | |
| Concatenated | MAGTSRTLSYYKLGASQRVAGDSGFAAYSRYRIGNYCAAGTTQTAC | 58 |
| EDGE predicted | TDDNALAYYNTTKGGWPQIAQFAPSASAFFGMSRIGMEVTPSGTWLT | |
| SARS-CoV-2 | YTGAIKLDDKDPNPANNAAIVLQLPQGTTLPKGFYAEGGVPINTNSSP | |
| MHC Class I | DDQIGYYRRATRRIRLYLYALVYFLQSINFVRIIMRLWLCSTDVVYRA | |
| Epitope Cassette | FDIYNDKVAGFAKFLKTRQKRTATKAYNVTQAFGRRGPEQTQPYVC | |
| with N-term | NAPGCDVTDVTQLYLGGMSYYACLVGLMWLSYFIASFRLFARTRSM | |
| leader (bold) and | WSFNPETNILLNVPLHGTILTRPLLESELVILLNKHIDAYKTFPPTEPKK | |
| C-term | DKKKKADETQALPQRQKKQQTVTVGDSAEVAVKMFDAYVNTFSSTF | |
| Universal MHC | NVMDLFMRIFTIGTVTLKQGEIKDATPSDFVRATATIPIQASLPFGWLI | |
| Class II with | LLQFAYANRNRFLYIIKLIFLWLLWPVLAVFQSASKIITLKKRWQLALS | |
| GPGPG linkers | KGVHFVCNLLLLHVMSKHTDFSSEIIGYKAIDGGVTRDCVVLHSYFTS | |
| (SEQ ID NO: | DYYQLYSTQLSTDTGVEHVTFFIYNKIVDEPEEHVQIHTIDGSSGVCNI | |
| 56) (bold italic) | VNVSLVKPSFYVYSRVKNLNSSRVPGPGPGAKFVAAWTLKAAAGPGP | |
| SARS-CoV-2 | GQYIKANSKFIGITELGPGPG | |
| Spike | MFVFLVLLPLVSSQCVNLTTRTQLPPAYTNSFTRGVYYPDKVFRSSVL | 59 |
| | HSTQDLFLPFFSNVTWFHAIHVSGTNGTKRFDNPVLPFNDGVYFASTE | |
| | KSNIIRGWIFGTTLDSKTQSLLIVNNATNVVIKVCEFQFCNDPFLGVYY | |
| | HKNNKSWMESEFRVYSSANNCTFEYVSQPFLMDLEGKQGNFKNLRE | |
| | FVFKNIDGYFKIYSKHTPINLVRDLPQGFSALEPLVDLPIGINITRFQTLL | |
| | ALHRSYLTPGDSSSGWTAGAAAYYVGYLQPRTFLLKYNENGTITDAV | |
| | DCALDPLSETKCTLKSFTVEKGIYQTSNFRVQPTESIVRFPNITNLCPFG | |
| | EVFNATRFASVYAWNRKRISNCVADYSVLYNSASFSTFKCYGVSPTK | |
| | LNDLCFTNVYADSFVIRGDEVRQIAPGQTGKIADYNYKLPDDFTGCVI | |
| | AWNSNNLDSKVGGNYNYLYRLFRKSNLKPFERDISTEIYQAGSTPCN | |
| | GVEGFNCYFPLQSYGFQPTNGVGYQPYRVVVLSFELLHAPATVCGPK | |
| | KSTNLVKNKCVNFNFNGLTGTGVLTESNKKFLPFQQFGRDIADTTDA | |
| | VRDPQTLEILDITPCSFGGVSVITPGTNTSNQVAVLYQDVNCTEVPVAI | |
| | HADQLTPTWRVYSTGSNVFQTRAGCLIGAEHVNNSYECDIPIGAGICA | |
| | SYQTQTNSPRRARSVASQSIIAYTMSLGAENSVAYSNNSIAIPTNFTISV | |
| | TTEILPVSMTKTSVDCTMYICGDSTECSNLLLQYGSFCTQLNRALTGIA | |
| | VEQDKNTQEVFAQVKQIYKTPPIKDFGGFNFSQILPDPSKPSKRSFIED | |
| | LLFNKVTLADAGFIKQYGDCLGDIAARDLICAQKENGLTVLPPLLTDE | |
| | MIAQYTSALLAGTITSGWTFGAGAALQIPFAMQMAYRENGIGVTQNV | |
| | LYENQKLIANQFNSAIGKIQDSLSSTASALGKLQDVVNQNAQALNTL | |
| | VKQLSSNFGAISSVLNDILSRLDKVEAEVQIDRLITGRLQSLQTYVTQQ | |
| | LIRAAEIRASANLAATKMSECVLGQSKRVDFCGKGYHLMSFPQSAPH | |
| | GVVFLHVTYVPAQEKNFTTAPAICHDGKAHFPREGVFVSNGTHWFVT | |
| | QRNFYEPQIITTDNTFVSGNCDVVIGIVNNTVYDPLQPELDSFKEELDK | |
| | YFKNHTSPDVDLGDISGINASVVNIQKEIDRLNEVAKNLNESLIDLQEL | |
| | GKYEQYIKWPWYIWLGFIAGLIAIVMVTIMLCCMTSCCSCLKGCCSC | |
| | GSCCKFDEDDSEPVLKGVKLHYT | |
| SARS-CoV-2 | MFVFLVLLPLVSSQCVNLTTRTQLPPAYTNSFTRGVYYPDKVFRSSVL | 60 |
| Modified Spike | HSTQDLFLPFFSNVTWFHAIHVSGTNGTKRFDNPVLPFNDGVYFASTE | |
| (Potential | KSNIIRGWIFGTTLDSKTQSLLIVNNATNVVIKVCEFQFCNDPFLGVYY | |
| mutations in | HKNNKSWMESEFRVYSSANNCTFEYVSQPFLMDLEGKQGNFKNLRE | |
| Bold italic | FVFKNIDGYFKIYSKHTPINLVRDLPQGFSALEPLVDLPIGINITRFQTLL | |
| lowercase. | ALHRSYLTPGDSSSGWTAGAAAYYVGYLQPRTFLLKYNENGTITDAV | |
| modifications | DCALDPLSETKCTLKSFTVEKGIYQTSNFRVQPTESIVRFPNITNLCPFG | |
| may be in | EVFNATRFASVYAWNRKRISNCVADYSVLYNSASFSTFKCYGVSPTK | |
| separate or | LNDLCFTNVYADSFVIRGDEVRQIAPGQTGKIADYNYKLPDDFTGCVI | |
| combined | AWNSNNLDSKVGGNYNYLYRLFRKSNLKPFERDISTEIYQAGSTPCN | |
| constructs) | GVEGFNCYFPLQSYGFQPTNGVGYQPYRVVVLSFELLHAPATVCGPK | |
| | KSTNLVKNKCVNFNFNGLTGTGVLTESNKKFLPFQQFGRDIADTTDA | |
| | VRDPQTLEILDITPCSFGGVSVITPGTNTSNQVAVLYQgVNCTEVPVAI | |
| | HADQLTPTWRVYSTGSNVFQTRAGCLIGAEHVNNSYECDIPIGAGICA | |
| | SYQTQTNSPvRARSVASQSIIAYTMSLGAENSVAYSNNSIAIPTNFTISV | |
| | TTEILPVSMTKTSVDCTMYICGDSTECSNLLLQYGSFCTQLNRALTGIA | |
| | VEQDKNTQEVFAQVKQIYKTPPIKDFGGFNFSQILPDPSKPSKnSFIEDL | |
| | LFNKVTLADAGFIKQYGDCLGDIAARDLICAQKFNGLTVLPPLLTDEM | |
| | IAQYTSALLAGTITSGWTFGAGAALQIPFAMQMAYRENGIGVTQNVL | |
| | YENQKLIANQFNSAIGKIQDSLSSTASALGKLQDVVNQNAQALNTLV | |
| | KQLSSNFGAISSVLNDILSRLDppEAEVQIDRLITGRLQSLQTYVTQQLI | |
| | RAAEIRASANLAATKMSECVLGQSKRVDFCGKGYHLMSFPQSAPHG | |
| | VVFLHVTYVPAQEKNFTTAPAICHDGKAHFPREGVFVSNGTHWFVTQ | |
| | RNFYEPQIITTDNTFVSGNCDVVIGIVNNTVYDPLQPELDSFKEELDKY | |
| | FKNHTSPDVDLGDISGINASVVNIQKEIDRLNEVAKNLNESLIDLQELG | |
| | KYEQYIKWPWYIWLGFIAGLIAIVMVTIMLCCMTSCCSCLKGCCSCGS | |
| | CCKFDEDDSEPVLKGVKLHYT | |
| SARS-CoV-2 | MADSNGTITVEELKKLLEQWNLVIGFLFLTWICLLQFAYANRNRFLYI | 61 |
| Membrane | IKLIFLWLLWPVTLACFVLAAVYRINWITGGIAIAMACLVGLMWLSYF | |
| | IASFRLFARTRSMWSFNPETNILLNVPLHGTILTRPLLESELVIGAVILR | |
| | GHLRIAGHHLGRCDIKDLPKEITVATSRTLSYYKLGASQRVAGDSGFA | |
| | AYSRYRIGNYKLNTDHSSSSDNIALLVQ | |
| SARS-CoV-2 | MSDNGPQNQRNAPRITFGGPSDSTGSNQNGERSGARSKQRRPQGLPN | 62 |
| Nucleocapsid | NTASWFTALTQHGKEDLKFPRGQGVPINTNSSPDDQIGYYRRATRRIR | |
| | GGDGKMKDLSPRWYFYYLGTGPEAGLPYGANKDGIIWVATEGALNT | |
| | PKDHIGTRNPANNAAIVLQLPQGTTLPKGFYAEGSRGGSQASSRSSSR | |
| | SRNSSRNSTPGSSRGTSPARMAGNGGDAALALLLLDRLNQLESKMSG | |
| | KGQQQQGQTVTKKSAAEASKKPRQKRTATKAYNVTQAFGRRGPEQT | |
| | QGNFGDQELIRQGTDYKHWPQIAQFAPSASAFFGMSRIGMEVTPSGT | |
| | WLTYTGAIKLDDKDPNFKDQVILLNKHIDAYKTFPPTEPKKDKKKKA | |
| | DETQALPQRQKKQQTVTLLPAADLDDFSKOLQQSMSSADSTQA | |
| SARS-CoV-2 | MYSFVSEETGTLIVNSVLLFLAFVVFLLVTLAILTALRLCAYCCNIVNV | 63 |
| Envelope | SLVKPSFYVYSRVKNLNSSRVPDLLV | |

TABLE 4
SARS-CoV-2 Vaccine Designs

| | SEQ |
|---|---|
| Vector Insert | ID NO: |
| EDGE Predicted Epitopes (EPE): Concatenated EDGE predicted | 64 |
| SARS-CoV-2 MHC Class I Epitope Cassette with 5′ expression | |
| leader (bold) and 3′ Universal MHC Class II with GPGPG | |
| linkers (SEQ ID NO: 56) (bold italic) | |
| CMV-EPE-BGH | 65 |
| CMV-Spike (IDT)-T2A-membrane protein-SV40 | 66 |
| CMV-Spike (IDT)-T2A-membrane protein-BGH | 67 |
| CMV-Spike (IDT)-SV40-CMV-EPE-BGH | 68 |
| CMV-Spike (IDT)-SV40 | 69 |
| CMV-Modified Spike (IDT)-SV40 | 70 |
| CMV-Spike (IDT)-T2A-Envelope-SV40-CMV-nucleocapsid- | 71 |
| T2A-membrane protein-SV40 | |
| CMV-Spike (IDT)-SV40 -CMV-nucleocapsid-T2A-membrane | 72 |
| protein-BGH | |
| CMV-Spike (IDT)-T2A-envelope protein-T2A-membrane | 73 |
| protein-SV40 | |
| CMV-Spike (IDT)-T2A-envelope protein-T2A-membrane | 74 |
| protein-SV40-CMV-EPE-BGH | |

### SAM Vectors

A RNA alphavirus backbone for the antigen expression system was generated from a self-replicating Venezuelan Equine Encephalitis (VEE) virus (Kinney, 1986. Virology 152: 400-413) by deleting the structural proteins of VEE located 3′ of the 26S sub-genomic promoter (VEE sequences 7544 to 11,175 deleted; numbering based on Kinney et al 1986; SEQ ID NO:6). To generate the self-amplifying mRNA (“SAM”) vector, the deleted sequences are replaced by antigen sequences. A representative SAM vector containing 20 model antigens is “VEE-MAG25mer” (SEQ ID NO:4). The vectors featuring the antigen cassettes described having the MAG25mer cassette can be replaced by the SARS-COV-2 cassettes and/or full-length proteins described herein.

### In Vitro Transcription to Generate SAM

For in vivo studies: SAM vectors were generated as “AU-SAM” vectors. A modified T7 RNA polymerase promoter (TAATACGACTCACTATA; SEQ ID NO: 120), which lacks the canonical 3′ dinucleotide GG, was added to the 5′ end of the SAM vector to generate the in vitro transcription template DNA (SEQ ID NO:77; 7544 to 11,175 deleted without an inserted antigen cassette). Reaction conditions are described below:
1× transcription buffer (40 mM Tris-HCL [pH7.9], 10 mM dithiothreitol, 2 mM spermidine, 0.002% Triton X-100, and 27 mM magnesium chloride) using final concentrations of 1×T7 RNA polymerase mix (E2040S); 0.025 mg/mL DNA transcription template (linearized by restriction digest); 8 mM CleanCap Reagent AU (Cat. No. N-7114) and 10 mM each of ATP, cytidine triphosphate (CTP), GTP, and uridine triphosphate (UTP)
Transcription reactions were incubated at 37° C. for 2 hr and treated with final 2 U DNase I (AM2239)/0.001 mg DNA transcription template in DNase I buffer for 1 hr at 37° C.
SAM was purified by RNeasy Maxi (QIAGEN, 75162)

Alternatively to co-transcriptional addition of a 5′ cap structure, a 7-methylguanosine or a related 5′ cap structure can be enzymatically added following transcription using a vaccinia capping system (NEB Cat. No. M2080) containing mRNA 2′-O-methyltransferase and S-Adenosyl methionine.

### Adenoviral Vectors

A modified ChAdV68 vector (“chAd68-Empty-E4deleted” SEQ ID NO:75) for the antigen expression system was generated based on AC_000011.1 with E1 (nt 577 to 3403), E3 (nt 27,125-31,825), and E4 region (nt 34,916 to 35,642) sequences deleted and the corresponding ATCC VR-594 (Independently sequenced Full-Length VR-594 C68 SEQ ID NO:10) nucleotides substituted at five positions. The full-length ChAdV68 AC_000011.1 sequence with corresponding ATCC VR-594 nucleotides substituted at five positions is referred to as “ChAdV68.5WTnt” (SEQ ID NO:1). Antigen cassettes under the control of the CMV promoter/enhancer are inserted in place of deleted E1 sequences.

### Adenoviral Production in 293F Cells

ChAdV68 virus production are performed in 293F cells grown in 293 FreeStyle™ (ThermoFisher) media in an incubator at 8% C02. On the day of infection cells are diluted to 10⁶ cells per mL, with 98% viability and 400 mL are used per production run in IL Shake flasks (Corning). 4 mL of the tertiary viral stock with a target MOI of >3.3 is used per infection. The cells are incubated for 48-72h until the viability was <70% as measured by Trypan blue. The infected cells are then harvested by centrifugation, full speed bench top centrifuge and washed in 1×PBS, re-centrifuged and then re-suspended in 20 mL of 10 mM Tris pH7.4. The cell pellet is lysed by freeze thawing 3× and clarified by centrifugation at 4,300×g for 5 minutes.

### Adenoviral Purification by CsCl Centrifugation

Viral DNA is purified by CsCl centrifugation. Two discontinuous gradient runs are performed. The first to purify virus from cellular components and the second to further refine separation from cellular components and separate defective from infectious particles.

10 mL of 1.2 (26.8g CsCl dissolved in 92 mL of 10 mM Tris pH 8.0) CsCl is added to polyallomer tubes. Then 8 mL of 1.4 CsCl (53g CsCl dissolved in 87 mL of 10 mM Tris pH 8.0) is carefully added using a pipette delivering to the bottom of the tube. The clarified virus is carefully layered on top of the 1.2 layer. If needed more 10 mM Tris is added to balance the tubes. The tubes are then placed in a SW-32Ti rotor and centrifuged for 2h 30 min at 10ºC. The tube are then removed to a laminar flow cabinet and the virus band pulled using an 18 gauge needle and a 10 ml syringe. Care is taken not to remove contaminating host cell DNA and protein. The band is then diluted at least 2× with 10 mM Tris pH 8.0 and layered as before on a discontinuous gradient as described above. The run is performed as described before except that this time the run is performed overnight. The next day the band is pulled with care to avoid pulling any of the defective particle band. The virus is then dialyzed using a Slide-a-Lyzer™ Cassette (Pierce) against ARM buffer (20 mM Tris pH 8.0, 25 mM NaCl, 2.5% Glycerol). This is performed 3×, 1 h per buffer exchange. The virus is then aliquoted for storage at −80° C.

### Adenoviral Viral Assays

VP concentration is performed by using an OD 260 assay based on the extinction coefficient of 1.1×10¹² viral particles (VP) is equivalent to an Absorbance value of 1 at OD260 nm. Two dilutions (1:5 and 1:10) of adenovirus are made in a viral lysis buffer (0.1% SDS, 10 mM Tris pH 7.4, 1 mM EDTA). OD is measured in duplicate at both dilutions and the VP concentration/mL is measured by multiplying the OD260 value X dilution factor X 1.1×10¹²VP.

An infectious unit (IU) titer is calculated by a limiting dilution assay of the viral stock. The virus is initially diluted 100× in DMEM/5% NS/1×PS and then subsequently diluted using 10-fold dilutions down to 1×10⁻⁷. 100 μL of these dilutions are then added to 293A cells that were seeded at least an hour before at 3e5 cells/well of a 24 well plate. This is performed in duplicate. Plates are incubated for 48h in a CO2 (5%) incubator at 37° C. The cells are then washed with 1×PBS and are then fixed with 100% cold methanol (−20° C.). The plates are then incubated at −20° C. for a minimum of 20 minutes. The wells are washed with 1×PBS then blocked in 1×PBS/0.1% BSA for 1 h at room temperature. A rabbit anti-Ad antibody (Abcam, Cambridge, MA) is added at 1:8,000 dilution in blocking buffer (0.25 ml per well) and incubated for 1 h at room temperature. The wells are washed 4× with 0.5 mL PBS per well. A HRP conjugated Goat anti-Rabbit antibody (Bethyl Labs, Montgomery Texas) diluted 1000× is added per well and incubated for 1 h prior to a final round of washing. 5 PBS washes are performed and the plates were developed using DAB (Diaminobenzidine tetrahydrochloride) substrate in Tris buffered saline (0.67 mg/mL DAB in 50 mM Tris pH 7.5, 150 mM NaCl) with 0.01% H₂O₂. Wells are developed for 5 min prior to counting. Cells are counted under a 10× objective using a dilution that gave between 4-40 stained cells per field of view. The field of view that is used was a 0.32 mm2 grid of which there are equivalent to 625 per field of view on a 24 well plate. The number of infectious viruses/mL can be determined by the number of stained cells per grid multiplied by the number of grids per field of view multiplied by a dilution factor 10. Similarly, when working with GFP expressing cells florescent can be used rather than capsid staining to determine the number of GFP expressing virions per mL.

### XIV.C. Spike Protein Sequence Optimization

Various sequence-optimized nucleotide sequences encoding the Spike protein were evaluated in ChAdV68 vaccine vectors.

### Sequence-Optimization of Spike Sequence

The Spike nucleotide sequence from Wuhan Hu/1 (SEQ ID NO:78) was sequence-optimized by substituting synonymous codons such that the amino acid sequence was unaffected. An IDT algorithm was used for enhanced expression in humans and for reduced complexity to aid synthesis (see, e.g., SEQ ID NOs:66-74). The Spike sequence was additionally sequence-optimized using two additional algorithms; (1) a single sequence (SEQ ID NO:87) generated using SGI DNA (La Jolla, CA); (2) 6 sequences designated CT1, CT20, CT56, CT83, CT131, and CT 199 (SEQ ID NOs:79-84) generated using COOL (COOL algorithm generates multiple sequences and 6 were selected). The sequences of each are presented in Table 5.

Splicing events were identified in cDNA from 293A cells infected with ChAdV68 viruses or transfected with ChAdV68 genomic DNA. Specifically, total RNA from 10e5-10e6 cells was purified using Qiagen's RNeasy columns. Residual DNA was removed by DNAse treatment, and cDNA was produced using SuperScriptIV reverse transcriptase (Thermo). Subsequently, primers specific for the 5′ UTR and 3′ UTR of the Gritstone ChAdV68 cassette were used to generate PCR products, analyzed by agarose gel electrophoresis, gel-purified, and Sanger-sequenced to identify regions deleted by splicing.

Splice donor sites were removed by site-directed mutagenesis disrupting the nucleotide sequence motif while not disturbing the amino acid sequence. Mutagenesis was accomplished by incorporating above mutations into PCR primers, amplifying several fragments in parallel, and running a Gibson assembly on the fragments (overlapping by 30-60 nt). Optimized clone CT1-2C (SEQ ID NO:85) had Sanger sequence-identified splice donor motifs at NT385 and NT539 mutated, and clone IDT-4C (SEQ ID NO:86) had Sanger sequence-identified splice donor motifs at NT385, NT539, and predicted donor motifs at NT2003, and NT2473 mutated. Additionally, a possible polyadenylation site AATAAA at nt 445 was mutated to AAcAAA in IDT-4C clone.

The sequences described are presented in Table 5.

TABLE 5
Sequence-optimized Spike Sequences

| | | SEQ |
|---|---|---|
| Spike | | ID |
| Sequence | Nucleic Acid Sequence | NO: |
| Spike Native; | atgtttgtttttcttgttttattgccactagtctctagtcagtgtgttaatcttacaaccagaactcaattaccccctgc | 78 |
| NC_045512.2 | atacactaattctttcacacgtggtgtttattaccctgacaaagttttcagatcctcagttttacattcaactcaggactt | |
| Severe acute | gttcttacctttcttttccaatgttacttggttccatgctatacatgtctctgggaccaatggtactaagaggtttgata | |
| respiratory | accctgtcctaccatttaatgatggtgtttattttgcttccactgagaagtctaacataataagaggctggatttttgg | |
| syndrome | tactactttagattcgaagacccagtccctacttattgttaataacgctactaatgttgttattaaagtctgtgaatttc | |
| coronavirus | aattttgtaatgatccatttttgggtgtttattaccacaaaaacaacaaaagttggatggaaagtgagttcagagttt | |
| 2 isolate | attctagtgcgaataattgcacttttgaatatgtctctcagccttttcttatggaccttgaaggaaaacagggtaattt | |
| Wuhan-Hu-1 | caaaaatcttagggaatttgtgtttaagaatattgatggttattttaaaatatattctaagcacacgcctattaatttag | |
| | tgcgtgatctccctcagggtttttcggctttagaaccattggtagatttgccaataggtattaacatcactaggtttca | |
| | aactttacttgctttacatagaagttatttgactcctggtgattcttcttcaggttggacagctggtgctgcagcttatt | |
| | atgtgggttatcttcaacctaggacttttctattaaaatataatgaaaatggaaccattacagatgctgtagactgtg | |
| | cacttgaccctctctcagaaacaaagtgtacgttgaaatccttcactgtagaaaaaggaatctatcaaacttctaa | |
| | ctttagagtccaaccaacagaatctattgttagatttcctaatattacaaacttgtgcccttttggtgaagtttttaacg | |
| | ccaccagatttgcatctgtttatgcttggaacaggaagagaatcagcaactgtgttgctgattattctgtcctatata | |
| | attccgcatcattttccacttttaagtgttatggagtgtctcctactaaattaaatgatctctgctttactaatgtctat | |
| | gcagattcatttgtaattagaggtgatgaagtcagacaaatcgctccagggcaaactggaaagattgctgattata | |
| | attataaattaccagatgattttacaggctgcgttatagcttggaattctaacaatcttgattctaaggttggtggtaa | |
| | ttataattacctgtatagattgtttaggaagtctaatctcaaaccttttgagagagatatttcaactgaaatctatcag | |
| | gccggtagcacaccttgtaatggtgttgaaggttttaattgttactttcctttacaatcatatggtttccaacccacta | |
| | atggtgttggttaccaaccatacagagtagtagtactttcttttgaacttctacatgcaccagcaactgtttgtggac | |
| | ctaaaaagtctactaatttggttaaaaacaaatgtgtcaatttcaacttcaatggtttaacaggcacaggtgttctta | |
| | ctgagtctaacaaaaagtttctgcctttccaacaatttggcagagacattgctgacactactgatgctgtccgtgat | |
| | ccacagacacttgagattcttgacattacaccatgttcttttggtggtgtcagtgttataacaccaggaacaaatac | |
| | ttctaaccaggttgctgttctttatcaggatgttaactgcacagaagtccctgttgctattcatgcagatcaacttact | |
| | cctacttggcgtgtttattctacaggttctaatgtttttcaaacacgtgcaggctgtttaataggggctgaacatgtc | |
| | aacaactcatatgagtgtgacatacccattggtgcaggtatatgcgctagttatcagactcagactaattctcctc | |
| | ggcgggcacgtagtgtagctagtcaatccatcattgcctacactatgtcacttggtgcagaaaattcagttgctta | |
| | ctctaataactctattgccatacccacaaattttactattagtgttaccacagaaattctaccagtgtctatgaccaa | |
| | gacatcagtagattgtacaatgtacatttgtggtgattcaactgaatgcagcaatcttttgttgcaatatggcagtttt | |
| | tgtacacaattaaaccgtgctttaactggaatagctgttgaacaagacaaaaacacccaagaagtttttgcacaa | |
| | gtcaaacaaatttacaaaacaccaccaattaaagattttggtggttttaatttttcacaaatattaccagatccatcaa | |
| | aaccaagcaagaggtcatttattgaagatctacttttcaacaaagtgacacttgcagatgctggcttcatcaaaca | |
| | atatggtgattgccttggtgatattgctgctagagacctcatttgtgcacaaaagtttaacggccttactgttttgcc | |
| | acctttgctcacagatgaaatgattgctcaatacacttctgcactgttagcgggtacaatcacttctggttggacctt | |
| | tggtgcaggtgctgcattacaaataccatttgctatgcaaatggcttataggtttaatggtattggagttacacaga | |
| | atgttctctatgagaaccaaaaattgattgccaaccaatttaatagtgctattggcaaaattcaagactcactttcttc | |
| | cacagcaagtgcacttggaaaacttcaagatgtggtcaaccaaaatgcacaagctttaaacacgcttgttaaac | |
| | aacttagctccaattttggtgcaatttcaagtgttttaaatgatatcctttcacgtcttgacaaagttgaggctgaagt | |
| | gcaaattgataggttgatcacaggcagacttcaaagtttgcagacatatgtgactcaacaattaattagagctgca | |
| | gaaatcagagcttctgctaatcttgctgctactaaaatgtcagagtgtgtacttggacaatcaaaaagagttgattt | |
| | ttgtggaaagggctatcatcttatgtccttccctcagtcagcacctcatggtgtagtcttcttgcatgtgacttatgtc | |
| | cctgcacaagaaaagaacttcacaactgctcctgccatttgtcatgatggaaaagcacactttcctcgtgaaggt | |
| | gtctttgtttcaaatggcacacactggtttgtaacacaaaggaatttttatgaaccacaaatcattactacagacaa | |
| | cacatttgtgtctggtaactgtgatgttgtaataggaattgtcaacaacacagtttatgatcctttgcaacctgaatta | |
| | gactcattcaaggaggagttagataaatattttaagaatcatacatcaccagatgttgatttaggtgacatctctgg | |
| | cattaatgcttcagttgtaaacattcaaaaagaaattgaccgcctcaatgaggttgccaagaatttaaatgaatctc | |
| | tcatcgatctccaagaacttggaaagtatgagcagtatataaaatggccatggtacatttggctaggttttatagct | |
| | ggcttgattgccatagtaatggtgacaattatgctttgctgtatgaccagttgctgtagttgtctcaagggctgttgtt | |
| | cttgtggatcctgctgcaaatttgatgaagacgactctgagccagtgctcaaaggagtcaaattacattacacata | |
| | a | |
| Spike CT1 | ATGTTTGTCTTCCTGGTCTTGCTGCCGCTGGTGAGCAGCCAGTGCG | 79 |
| Optimized | TGAATCTCACCACCCGCACCCAGCTTCCACCTGCCTACACTAACAG | |
| | CTTCACCCGAGGGGTGTATTACCCTGACAAGGTATTCCGGTCCTCC | |
| | GTCCTCCATAGCACGCAGGACCTTTTTCTGCCCTTCTTCTCAAATGT | |
| | GACATGGTTCCATGCCATTCACGTGAGCGGCACGAATGGAACGAA | |
| | GCGCTTTGATAACCCCGTGCTGCCTTTCAATGACGGCGTCTACTTC | |
| | GCCTCCACTGAAAAGTCAAACATCATCCGGGGCTGGATCTTTGGC | |
| | ACCACTCTTGATTCAAAGACCCAGTCACTGCTGATTGTGAACAATG | |
| | CTACAAACGTGGTTATCAAGGTGTGTGAGTTTCAGTTCTGTAACGA | |
| | TCCATTTTTGGGAGTGTACTACCACAAGAACAACAAGTCCTGGATG | |
| | GAGTCTGAGTTCAGAGTGTATAGCTCTGCTAACAACTGCACCTTCG | |
| | AGTACGTGTCCCAGCCTTTCCTTATGGACCTGGAAGGCAAACAGG | |
| | GCAATTTCAAAAACCTGAGAGAGTTCGTGTTTAAGAACATTGACG | |
| | GATACTTCAAAATTTATTCTAAGCACACACCAATTAACTTAGTGCG | |
| | GGACCTACCCCAAGGCTTTAGCGCCCTAGAGCCCCTGGTTGACCTG | |
| | CCCATTGGGATCAATATAACAAGGTTCCAAACTCTACTGGCTCTGC | |
| | ATAGAAGTTATCTGACCCCAGGAGACAGCTCTAGTGGTTGGACCG | |
| | CCGGCGCAGCAGCCTACTATGTCGGGTACTTACAGCCACGCACGTT | |
| | CCTTCTGAAGTACAATGAGAACGGGACAATCACTGACGCAGTAGA | |
| | CTGTGCACTGGACCCGCTAAGCGAGACTAAGTGCACACTTAAATC | |
| | CTTCACGGTGGAGAAAGGCATTTATCAGACCTCTAACTTCAGGGTG | |
| | CAGCCAACAGAAAGCATTGTGCGATTCCCAAATATTACTAATCTTT | |
| | GCCCTTTCGGGGAGGTCTTTAATGCAACTAGATTCGCATCAGTCTA | |
| | TGCGTGGAACCGCAAACGCATTTCCAATTGTGTCGCAGACTACTCA | |
| | GTGCTGTACAACTCTGCCTCTTTCAGTACGTTCAAGTGTTACGGAG | |
| | TGTCACCCACTAAACTGAACGACCTGTGCTTTACAAATGTCTACGC | |
| | TGACTCCTTCGTGATTAGGGGAGACGAGGTGAGACAAATTGCCCC | |
| | CGGACAGACTGGGAAGATTGCCGACTACAATTATAAGCTTCCTGA | |
| | TGATTTCACTGGCTGTGTTATTGCCTGGAATAGTAACAATCTGGAT | |
| | AGCAAGGTGGGAGGCAACTATAACTACTTATATCGACTGTTTAGG | |
| | AAGAGTAATCTGAAACCATTTGAGCGGGATATTTCCACAGAAATT | |
| | TACCAGGCCGGGAGCACACCATGTAATGGGGTGGAGGGATTTAAT | |
| | TGTTACTTCCCACTCCAGAGCTATGGTTTCCAACCCACCAATGGAG | |
| | TGGGTTACCAGCCCTATAGAGTCGTGGTGCTTAGTTTTGAGCTGCT | |
| | TCACGCCCCAGCAACCGTCTGCGGTCCCAAAAAGTCGACCAATCT | |
| | CGTGAAAAACAAATGCGTAAACTTCAACTTTAACGGCTTAACAGG | |
| | AACCGGCGTGCTCACCGAAAGCAACAAGAAATTCCTTCCATTTCA | |
| | GCAATTCGGAAGGGACATCGCCGACACAACAGACGCGGTGAGGG | |
| | ACCCACAGACTCTGGAGATACTGGACATCACTCCTTGTTCGTTTGG | |
| | GGGCGTCTCGGTCATCACACCCGGGACTAATACTAGTAATCAGGT | |
| | AGCAGTTTTATATCAAGGCGTCAACTGTACCGAAGTACCTGTGGCC | |
| | ATACACGCTGATCAGCTAACGCCAACATGGCGAGTCTATTCCACC | |
| | GGCTCTAACGTTTTTCAGACCAGGGCTGGGTGCCTGATAGGGGCA | |
| | GAGCACGTCAATAATTCCTATGAGTGTGATATCCCCATAGGTGCGG | |
| | GGATCTGTGCCAGCTATCAAACCCAAACCAATTCACCAAGGCGAG | |
| | CACGGTCTGTGGCTTCTCAGAGCATAATTGCATATACAATGTCACT | |
| | GGGCGCTGAGAATAGCGTTGCATACTCTAATAACAGCATAGCCAT | |
| | TCCCACGAACTTTACTATCAGTGTGACAACCGAAATATTGCCAGTT | |
| | TCGATGACCAAAACTAGCGTGGATTGCACGATGTACATCTGTGGA | |
| | GACTCTACCGAATGCAGCAATCTGCTATTACAATATGGCAGCTTCT | |
| | GTACACAGTTAAATCGAGCCTTGACAGGCATCGCAGTGGAACAGG | |
| | ACAAAAATACTCAAGAGGTGTTTGCACAGGTGAAGCAAATCTACA | |
| | AAACGCCCCCCATTAAAGATTTTGGCGGGTTCAATTTTTCACAAAT | |
| | TCTCCCCGACCCGTCTAAGCCGAGTAAGCGGTCCTTCATCGAAGAT | |
| | CTGCTCTTTAACAAAGTAACCCTCGCCGATGCCGGCTTTATTAAGC | |
| | AGTATGGCGACTGCCTGGGGGATATAGCCGCTCGTGACCTGATTTG | |
| | CGCCCAGAAGTTCAATGGTCTGACCGTGTTGCCTCCTTTATTGACC | |
| | GATGAAATGATTGCCCAGTACACTAGTGCCCTGCTGGCCGGCACT | |
| | ATCACGTCTGGGTGGACCTTCGGAGCTGGTGCCGCCTTGCAGATAC | |
| | CTTTTGCAATGCAGATGGCCTATAGGTTTAATGGTATCGGAGTGAC | |
| | TCAGAACGTACTGTACGAGAACCAGAAGCTCATCGCTAATCAATT | |
| | TAACTCCGCTATCGGAAAAATCCAGGACAGCCTCTCTTCTACAGCT | |
| | AGCGCTCTGGGCAAACTGCAGGATGTCGTTAATCAGAATGCCCAG | |
| | GCCCTGAACACCTTGGTTAAACAACTATCTTCCAACTTCGGGGCCA | |
| | TATCCAGTGTGTTGAATGATATTCTCTCCCGCTTGGATAAGGTGGA | |
| | AGCTGAGGTGCAGATCGATCGCTTGATCACCGGCAGACTGCAGTC | |
| | CCTCCAGACATATGTAACTCAGCAGCTGATTAGAGCCGCCGAGAT | |
| | AAGGGCAAGTGCGAATCTGGCTGCCACCAAGATGAGCGAATGTGT | |
| | ATTGGGCCAGAGCAAACGAGTTGATTTTTGCGGTAAGGGGTATCA | |
| | TTTAATGTCTTTCCCTCAATCCGCACCTCATGGCGTAGTTTTCCTGC | |
| | ATGTGACTTATGTCCCGGCTCAGGAGAAGAATTTTACCACAGCCCC | |
| | CGCGATCTGCCATGACGGAAAGGCCCACTTCCCCCGGGAAGGCGT | |
| | GTTTGTATCCAATGGGACTCACTGGTTTGTCACTCAGCGAAATTTT | |
| | TATGAACCACAGATCATCACCACTGACAACACATTTGTTAGTGGA | |
| | AACTGCGATGTGGTCATCGGCATCGTGAATAACACTGTCTATGATC | |
| | CACTGCAACCTGAACTGGATTCTTTTAAAGAGGAACTCGACAAGT | |
| | ATTTTAAAAACCACACTAGCCCTGACGTGGATCTCGGTGACATTTC | |
| | TGGCATCAACGCTAGCGTAGTGAACATTCAGAAAGAGATAGATAG | |
| | ACTTAATGAGGTGGCCAAGAACCTCAACGAAAGTCTGATCGACCT | |
| | CCAGGAACTGGGGAAATACGAGCAGTACATTAAATGGCCTTGGTA | |
| | CATATGGCTGGGGTTCATTGCTGGGCTGATCGCAATAGTGATGGTG | |
| | ACCATAATGCTCTGTTGCATGACTAGCTGCTGCAGCTGCCTGAAGG | |
| | GCTGCTGTAGTTGTGGGTCATGTTGTAAGTTTGACGAAGATGATAG | |
| | CGAGCCTGTCCTTAAAGGAGTGAAGCTCCACTACACCTAG | |
| Spike CT20 | See Sequence Listing | 80 |
| Optimized | | |
| Spike CT56 | See Sequence Listing | 81 |
| Optimized | | |
| Spike CT83 | See Sequence Listing | 82 |
| Optimized | | |
| Spike CT131 | See Sequence Listing | 83 |
| Optimized | | |
| Spike CT199 | See Sequence Listing | 84 |
| Optimized | | |
| Spike CT1-2C | ATGTTTGTCTTCCTGGTCTTGCTGCCGCTcGTGtctAGCCAGTGCGTG | 85 |
| | AATCTCACCACCCGCACCCAGCTTCCACCTGCCTACACTAACAGCT | |
| | TCACCCGAGGGGTGTATTACCCTGACAAGGTATTCCGGTCCTCCGT | |
| | CCTCCATAGCACGCAGGACCTTTTTCTGCCCTTCTTCTCAAATGTG | |
| | ACATGGTTCCATGCCATTCACGTGAGCGGCACGAATGGAACGAAG | |
| | CGCTTTGATAACCCCGTGCTGCCTTTCAATGACGGCGTCTACTTCG | |
| | CCTCCACTGAAAAGTCAAACATCATCCGGGGCTGGATCTTTGGCAC | |
| | CACTCTTGATTCAAAGACCCAGTCACTGCTGATTGTGAACAATGCT | |
| | ACAAACGTGGTTATCAAaGTcTGcGAGTTTCAGTTCTGTAACGATCC | |
| | ATTTTTGGGAGTGTACTACCACAAGAACAACAAGTCCTGGATGGA | |
| | GTCTGAGTTCAGAGTGTATAGCTCTGCTAACAACTGCACCTTCGAG | |
| | TACGTGTCCCAGCCTTTCCTTATGGACCTGGAAGGCAAACAGGGC | |
| | AATTTCAAAAACCTGAGAGAGTTCGTGTTTAAGAACATTGACGGA | |
| | TACTTCAAAATTTATTCTAAGCACACACCAATTAACTTAGTGCGGG | |
| | ACCTACCCCAAGGCTTTAGCGCCCTAGAGCCCCTGGTTGACCTGCC | |
| | CATTGGGATCAATATAACAAGGTTCCAAACTCTACTGGCTCTGCAT | |
| | AGAAGTTATCTGACCCCAGGAGACAGCTCTAGTGGTTGGACCGCC | |
| | GGCGCAGCAGCCTACTATGTCGGGTACTTACAGCCACGCACGTTCC | |
| | TTCTGAAGTACAATGAGAACGGGACAATCACTGACGCAGTAGACT | |
| | GTGCACTGGACCCGCTAAGCGAGACTAAGTGCACACTTAAATCCT | |
| | TCACGGTGGAGAAAGGCATTTATCAGACCTCTAACTTCAGGGTGC | |
| | AGCCAACAGAAAGCATTGTGCGATTCCCAAATATTACTAATCTTTG | |
| | CCCTTTCGGGGAGGTCTTTAATGCAACTAGATTCGCATCAGTCTAT | |
| | GCGTGGAACCGCAAACGCATTTCCAATTGTGTCGCAGACTACTCA | |
| | GTGCTGTACAACTCTGCCTCTTTCAGTACGTTCAAGTGTTACGGAG | |
| | TGTCACCCACTAAACTGAACGACCTGTGCTTTACAAATGTCTACGC | |
| | TGACTCCTTCGTGATTAGGGGAGACGAGGTGAGACAAATTGCCCC | |
| | CGGACAGACTGGGAAGATTGCCGACTACAATTATAAGCTTCCTGA | |
| | TGATTTCACTGGCTGTGTTATTGCCTGGAATAGTAACAATCTGGAT | |
| | AGCAAGGTGGGAGGCAACTATAACTACTTATATCGACTGTTTAGG | |
| | AAGAGTAATCTGAAACCATTTGAGCGGGATATTTCCACAGAAATT | |
| | TACCAGGCCGGGAGCACACCATGTAATGGGGTGGAGGGATTTAAT | |
| | TGTTACTTCCCACTCCAGAGCTATGGTTTCCAACCCACCAATGGAG | |
| | TGGGTTACCAGCCCTATAGAGTCGTGGTGCTTAGTTTTGAGCTGCT | |
| | TCACGCCCCAGCAACCGTCTGCGGTCCCAAAAAGTCGACCAATCT | |
| | CGTGAAAAACAAATGCGTAAACTTCAACTTTAACGGCTTAACAGG | |
| | AACCGGCGTGCTCACCGAAAGCAACAAGAAATTCCTTCCATTTCA | |
| | GCAATTCGGAAGGGACATCGCCGACACAACAGACGCcGTcAGGGA | |
| | CCCACAGACTCTGGAGATACTGGACATCACTCCTTGTTCGTTTGGG | |
| | GGCGTCTCGGTCATCACACCCGGGACTAATACTAGTAATCAGGTA | |
| | GCAGTTTTATATCAAGGCGTCAACTGTACCGAAGTACCTGTGGCCA | |
| | TACACGCTGATCAGCTAACGCCAACATGGCGAGTCTATTCCACCG | |
| | GCTCTAACGTTTTTCAGACCAGGGCTGGGTGCCTGATAGGGGCAG | |
| | AGCACGTCAATAATTCCTATGAGTGTGATATCCCCATAGGTGCGGG | |
| | GATCTGTGCCAGCTATCAAACCCAAACCAATTCACCAAGGCGAGC | |
| | ACGGTCTGTGGCTTCTCAGAGCATAATTGCATATACAATGTCACTG | |
| | GGCGCTGAGAATAGCGTTGCATACTCTAATAACAGCATAGCCATT | |
| | CCCACGAACTTTACTATCAGTGTGACAACCGAAATATTGCCAGTTT | |
| | CGATGACCAAAACTAGCGTGGATTGCACGATGTACATCTGTGGAG | |
| | ACTCTACCGAATGCAGCAATCTGCTATTACAATATGGCAGCTTCTG | |
| | TACACAGTTAAATCGAGCCTTGACAGGCATCGCAGTGGAACAGGA | |
| | CAAAAATACTCAAGAGGTGTTTGCACAGGTGAAGCAAATCTACAA | |
| | AACGCCCCCCATTAAAGATTTTGGCGGGTTCAATTTTTCACAAATT | |
| | CTCCCCGACCCGTCTAAGCCGAGTAAGCGGTCCTTCATCGAAGATC | |
| | TGCTCTTTAACAAAGTAACCCTCGCCGATGCCGGCTTTATTAAGCA | |
| | GTATGGCGACTGCCTGGGGGATATAGCCGCTCGTGACCTGATTTGC | |
| | GCCCAGAAGTTCAATGGTCTGACCGTGTTGCCTCCTTTATTGACCG | |
| | ATGAAATGATTGCCCAGTACACTAGTGCCCTGCTGGCCGGCACTAT | |
| | CACGTCTGGGTGGACCTTCGGAGCTGGTGCCGCCTTGCAGATACCT | |
| | TTTGCAATGCAGATGGCCTATAGGTTTAATGGTATCGGAGTGACTC | |
| | AGAACGTACTGTACGAGAACCAGAAGCTCATCGCTAATCAATTTA | |
| | ACTCCGCTATCGGAAAAATCCAGGACAGCCTCTCTTCTACAGCTAG | |
| | CGCTCTGGGCAAACTGCAGGATGTCGTTAATCAGAATGCCCAGGC | |
| | CCTGAACACCTTGGTTAAACAACTATCTTCCAACTTCGGGGCCATA | |
| | TCCAGTGTGTTGAATGATATTCTCTCCCGCTTGGATAAGGTGGAAG | |
| | CTGAGGTGCAGATCGATCGCTTGATCACCGGCAGACTGCAGTCCCT | |
| | CCAGACATATGTAACTCAGCAGCTGATTAGAGCCGCCGAGATAAG | |
| | GGCAAGTGCGAATCTGGCTGCCACCAAGATGAGCGAATGTGTATT | |
| | GGGCCAGAGCAAACGAGTTGATTTTTGCGGTAAGGGGTATCATTT | |
| | AATGTCTTTCCCTCAATCCGCACCTCATGGCGTAGTTTTCCTGCATG | |
| | TGACTTATGTCCCGGCTCAGGAGAAGAATTTTACCACAGCCCCCGC | |
| | GATCTGCCATGACGGAAAGGCCCACTTCCCCCGGGAAGGCGTGTT | |
| | TGTATCCAATGGGACTCACTGGTTTGTCACTCAGCGAAATTTTTAT | |
| | GAACCACAGATCATCACCACTGACAACACATTTGTTAGTGGAAAC | |
| | TGCGATGTGGTCATCGGCATCGTGAATAACACTGTCTATGATCCAC | |
| | TGCAACCTGAACTGGATTCTTTTAAAGAGGAACTCGACAAGTATTT | |
| | TAAAAACCACACTAGCCCTGACGTGGATCTCGGTGACATTTCTGGC | |
| | ATCAACGCTAGCGTAGTGAACATTCAGAAAGAGATAGATAGACTT | |
| | AATGAGGTGGCCAAGAACCTCAACGAAAGTCTGATCGACCTCCAG | |
| | GAACTGGGGAAATACGAGCAGTACATTAAATGGCCTTGGTACATA | |
| | TGGCTGGGGTTCATTGCTGGGCTGATCGCAATAGTGATGGTGACCA | |
| | TAATGCTCTGTTGCATGACTAGCTGCTGCAGCTGCCTGAAGGGCTG | |
| | CTGTAGTTGTGGGTCATGTTGTAAGTTTGACGAAGATGATAGCGAG | |
| | CCTGTCCTTAAAGGAGTGAAGCTCCACTACACCTAG | |
| Spike IDT-4C | See Sequence Listing | 86 |
| Spike-SGI | See Sequence Listing | 87 |

### Cloning of Sequence-Optimized Spike Sequences

Each sequence-optimized Spike sequence was ordered as a set of 3 gBlocks from IDT with each gblock between 1300-1500 bp and overlapping with each other by approximately 100 nucleotides. The gBlocks comprising the 5′ and 3′ ends of the Spike sequence overlapped with the plasmid backbone by 100 nucleotides. The gblocks were assembled by a combination of PCR and Gibson assembly into a linearized pA68-E4d AsisI/PmeI backbone to generate pA68-E4-sequence-optimized Spike clones. Clones were screened by PCR and clones of the correct size were then grown for plasmid production and sequencing by either NGS or Sanger sequencing. Once a correct clone was sequence confirmed, large scale plasmid production and purification was performed for transfection.

### Vector Production

pA68-E4-Spike plasmid DNA was digested with PacI and 2 μg DNA was transfected into 293F cells using TransIt Lenti transfection reagent. Five days post transfection, cells and media were harvested and a lysate generated by freeze-thawing at ˜80C and at 37 C. A fraction of the lysate was used to re-infect 30 mL of 293F cells and incubated for 48-72h before harvesting. Lysate was generated by freeze-thawing at ˜80C and at 37 C and a fraction of the lysate was used to infect 400 mL of 293F cells seeded at 1e6 cells/mL. Next, 48-72h later cells were harvested, lysed in 10 mM tris pH 8.0/0.1% Triton X-100 and freeze thawed 1× at 37 and ˜80C. The lysate was then clarified by centrifugation at 4300×g for 10 min prior to loading on a 1.2/1.4 CsCl gradient. The gradient was run for a minimum of 2h before the bands were harvested diluted 2-4× in Tris and then rerun on a 1.35 CsCl gradient for at least 2 h. The viral bands were harvested and then dialyzed 3× in 1× ARM buffer. The virus infectious titer was determined by an immunostaining titer assay and the viral particle measured by Absorbance at A260 nm.

### Western Analysis

Samples for Spike expression analysis were either harvested at designated times post transfection or in the case of purified virus by setting up a controlled infection experiment with a known virus MOI and harvested at a specific time post infection, typically 24 to 48h. 1e6 cells were typically harvested in 0.5 mL of SDS-PAGE loading buffer with 10% Beta-mercaptoethanol. Samples were boiled and run on 4-20% polyacrylamide gels under denaturing and reducing conditions. The gels were then blotted onto a PVDF membrane using a BioRad Rapid transfer device. The membrane was blocked for 2 h at room temperature in 5% Skim milk in TBST. The membrane was then probed with an anti-Spike SI polyclonal (Sino Biologicals) or anti-Spike monoclonal antibody 1A9 (GeneTex; Cat. No. GTX632604) and incubated for 2 h. The membrane was then washed in PBST (5×) and the probed with a HRP-linked anti-mouse antibody (Bethyl labs) for 1 h. The membrane was washed as described above and then incubated with a chemiluminescent substrate ECL plus (ThermoFisher). The image was then captured using a Chemidoc (BioRad device).

### Results

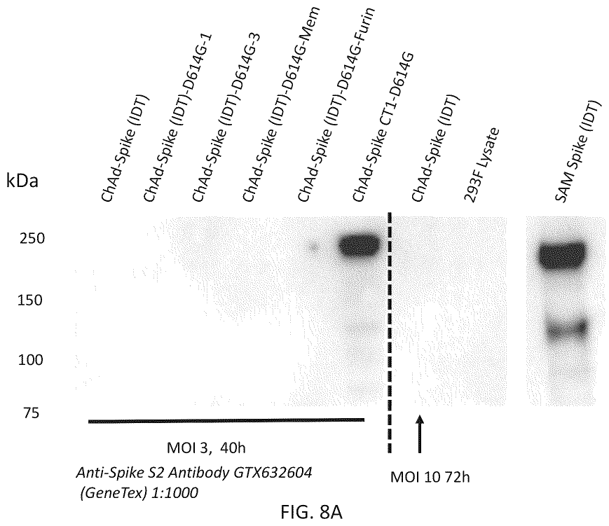

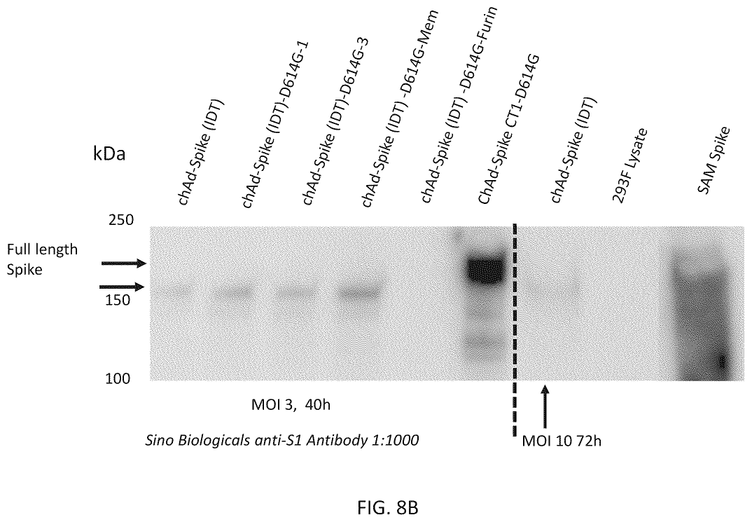

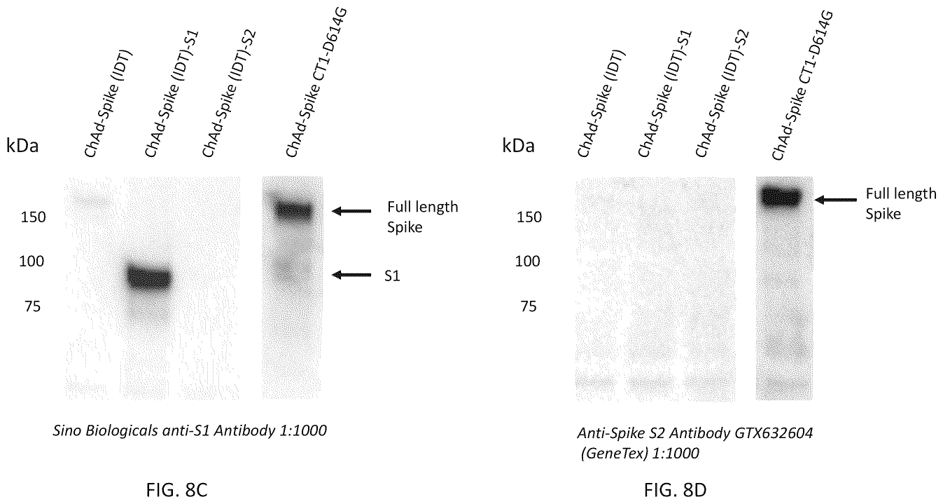

Expression of Spike S2 protein was assessed during viral production in 293F cells with various Spike-encoding vectors. As shown in FIG. 8A, using vectors encoding IDT sequence-optimized Spike cassettes, Spike S2 protein was detected by Western blot using an anti-Spike S2 antibody (GeneTex) when expressed in a SAM vector (FIG. 8A, last lane) but not when expressed in a ChAdV68 vector (“CMV-Spike (IDT)”; SEQ ID NO:69) at two different MOIs and timepoints (FIG. 8A, lanes 1 and 7). Two clones engineered to express Spike variant D614G (“CMV-Spike (IDT)-D614G” SEQ ID NO:70) also did not express detectable levels of Spike protein by Western using the S2 antibody (FIG. 8A, lanes 2 and 3). Clones engineered to co-express the SARS-COV-2 Membrane protein together with Spike (“CMV-Spike (IDT)-D614G-Mem” SEQ ID NO:66) or including a R682V mutations to disrupt the Furin cleavage site did not rescue the expression phenotype (FIG. 8A, lanes 4 and 5). In contrast, as shown in FIG. 8B, Spike SI protein was detected for all IDT constructs, albeit at low levels, with the exception of the Furin R682V mutation in which no Spike SI protein was detected.

To deconvolute if the expressions issues with the IDT sequence-optimized clones were specific to the SI or S2 domains, vectors expressing only the SI or S2 domains were also evaluated. As shown in FIG. 8C, a ChAdV68 vector encoding the IDT sequence-optimized Spike S1 protein alone demonstrated strong protein expression, in contrast to the lower expression observed with the full-length Spike vector (FIG. 8C, lanes 1 vs 2). As expected, no signal was observed for SI with the vector encoding the S2 domain alone. In contrast, as shown in FIG. 8D, a ChAdV68 vector encoding the IDT sequence-optimized Spike S2 protein alone did not demonstrate observable protein expression, comparable to the absence of signal observed with the full-length Spike vector (FIG. 8D, lanes 1 and 3). Thus, the data indicate the IDT sequence-optimized Spike S2 exhibited poor expression, including impacting expression of the full-length Spike sequence.

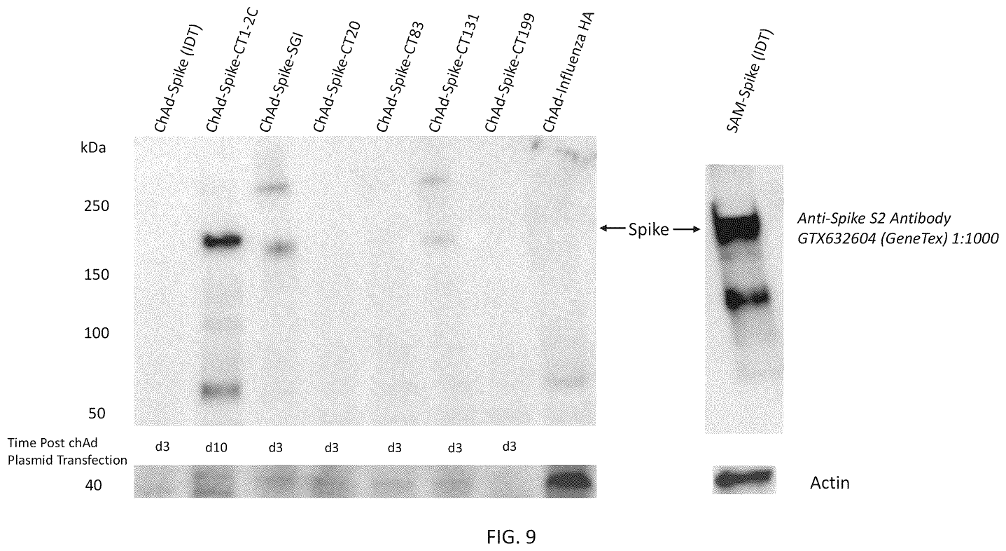

To address protein expression, the SARS-COV-2 Spike-encoding nucleotide sequence was sequence-optimized using additional sequence-optimization algorithms; (1) a single sequence (SEQ ID NO:87) generated using SGI DNA (La Jolla, CA); (2) 6 sequences designated CT1, CT20, CT56, CT83, CT131, and CT 199 (SEQ ID NOs:79-84) generated using COOL (COOL algorithm generates multiple sequences and 6 were selected). As shown in FIG. 8A and FIG. 8B, sequence—optimization with the COOL algorithm generated a sequence—CT1 (SEQ ID NO:79)—that demonstrated detectable expression using a ChAdV68 vector as assessed by Western using both an anti-S2 and anti-S1 antibody (FIG. 8A and FIG. 8B, each respective lane 6 “ChAd-Spike CT1-D614G”). The additional sequences generated using the COOL algorithm and the SGI algorithm were also assessed by Western. As shown in FIG. 9, the SGI clone and COOL sequence CT131 also demonstrated detectable levels of Spike protein by Western using an anti-S2 antibody (FIG. 9, lanes 3 and 6), while other COOL generated sequences did not generate detectable signals other than the control CT1 derived sequence (lane 2). Thus, the data indicate that specific sequence-optimizations improved expression of full-length SARS-COV-2 Spike protein in ChAdV68 vectors.

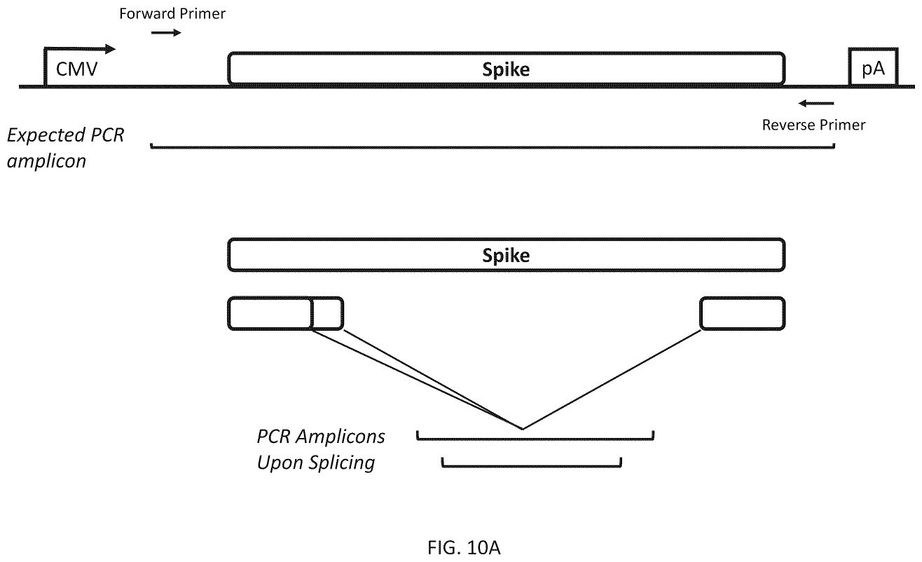

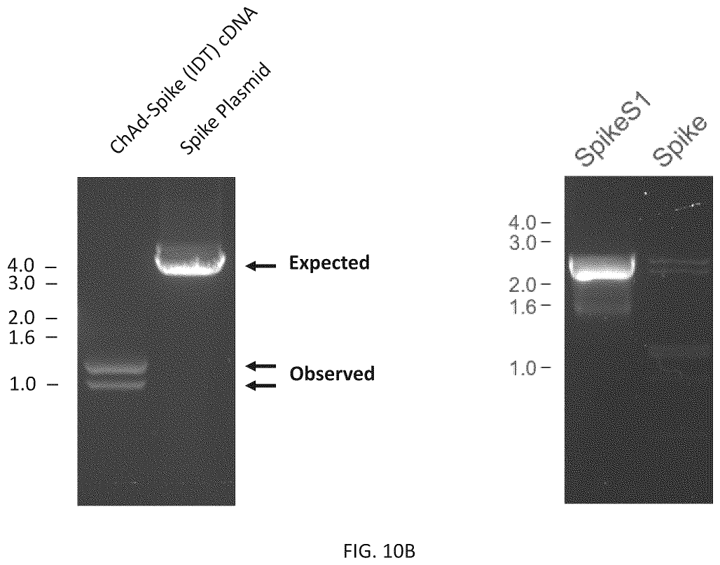

SARS-COV-2 is a cytoplasm-replicating positive-sense RNA virus encoding its own replication machinery, and as such SARS-COV-2 mRNA are not naturally processed by splicing and nuclear-export machineries. As illustrated in FIG. 10A, to assess the role of splicing in SARS-COV-2 Spike-encoding mRNA expressed from a ChAdV68 vector, primers were designed to amplify the Spike coding region. In the presence of mRNA splicing, amplicon sizes would be smaller than the expected full-length coding region. As shown in FIG. 10B, while PCR of the plasmid encoding the SARS-COV-2 Spike cassette demonstrated the expected amplicon size (“Spike Plasmid” left panel, right column), PCR amplification of cDNA from infected 293 cells demonstrated two smaller amplicons indicating splicing of the mRNA transcript (“ChAd-Spike (IDT) cDNA” left panel, left column). In addition, the Spike coding sequence was split into SI and S2 encoding sequences. As also shown in FIG. 10B, PCR amplification of SI cDNA from infected 293 cells demonstrated the expected amplicon size (“SpikeS1” right panel, left column) indicating SI was likely not undergoing undesired splicing while sequences in the S2 region may be influencing splicing.

The smaller amplicon sequences were analyzed and two splice donor sites were identified by Sanger sequencing. Three additional potential donor sites were predicted by further sequence analysis. The position and identity of the splice motif sequences are presented below (the nt triplets correspond to codons, numbering starts with reference to Spike ATG):

NT 385-:
AAG GTG TGT → AAa GTc TGc (identified by sequencing)
NT 539-:
AA GGT AAG C → Ag GGc AAa C (identified by sequencing)
NT 2003-:
CA GGT ATC T → Ct GGa ATC T (predicted)
NT 2473-:
AAG GTG ACC → AAa GTc ACC (predicted)
NT 3417-:
C CCC CTT CAG CCT GAA CTT GAT TCC (SEQ ID NO: 123) → T CCa CTg
CAa CCT GAA CTT GAT agt (SEQ ID NO: 124)

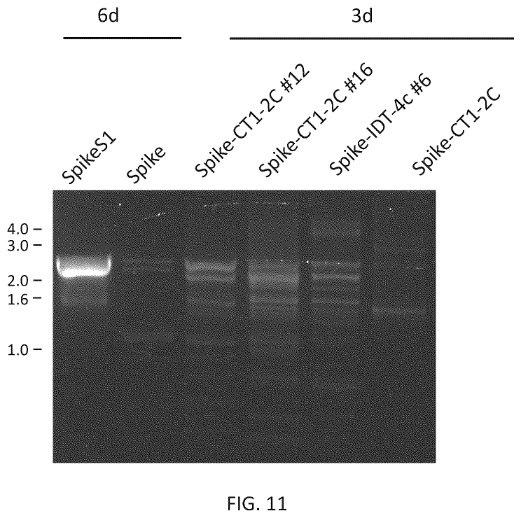

Selected splice donor sites were removed by site-directed mutagenesis disrupting the nucleotide sequence motif while not disturbing the amino acid sequence. COOL sequence-optimized clone CT1 was used as the reference sequence for clone CT1-2C (SEQ ID NO:85) having the sequence-identified splice donor motifs at NT385 and NT539 mutated. IDT sequence-optimized clone was used as the reference sequence for clone IDT-4C (SEQ ID NO:86) and had both sequence-identified and predicted splice donor motifs at NT385, NT539, NT2003, and NT2473 mutated, as well as a possible polyadenylation site AATAAA at NT445 mutated to AAcAAA. As shown in FIG. 9, Spike protein expression was detected by Western in the clone including the sequence-identified splice donor motifs (“CT1-2C” lane 2). Splicing was further assessed in the constructs by PCR analysis. As shown in FIG. 11, mutating the splice donor motifs and/or a potential polyA site alone did not prevent splicing indicating splicing potentially occurred from sub-dominant splice sites.

Given the identification of splicing events in the full-length Spike mRNA expressed from ChAdV68 vectors, additional constructs are generated and assessed for improved protein expression. Additional optimizations include constructs featuring exogenous nuclear export signals (e.g., Constitutive Transport Element (CTE), RNA Transport Element (RTE), or Woodchuck Posttranscriptional Regulatory Element (WPRE)) or the addition of an artificial intron through introduction of exogenous splice donor/branch/acceptor motif sequences to bias splicing, such as introducing a SV40 mini-intron (SEQ ID NO:88) between the CMV promoter and the Kozak sequence immediately upstream of the Spike gene. The identified and predicted splice donor motifs are also further evaluated in combination with additional sequence optimizations.

### XIV.D. SARS-COV-2 Vaccine Efficacy Evaluation

Various SARS-COV-2 vaccine designs, constructs, and dosing regimens were evaluated. The vaccines encoded various optimized versions of the Spike protein, selected predicted T cell epitopes (TCE), or a combination of Spike and TCE cassettes.

### Mouse Immunizations

All mouse studies were conducted at Murigenics under IACUC approved protocols. Balb/c mice (Envigo), 6-8 weeks old were used for all studies. Vaccines were stored at −80° C., thawed at room temperature on the day of immunization, and then diluted to 0.1 μg/mL with PBS and filtered through a 0.2 micron filter. Filtered formulations were stored at 4° C. and injected within 4 hours of preparation. All immunizations were bilateral intramuscular to the tibialis anterior, 2 injections of 50 μL each, 100 μL total.

### Non-Human Primate Immunizations

For SAM vaccines in Mamu-A*01 Indian rhesus macaques, SAM was administered as bilateral intramuscular injections into the quadriceps muscle at the indicated doses.

For ChAdV68 vaccines in Mamu-A*01 Indian rhesus macaques, ChAdV68 was administered bilaterally at the indicated doses (5×10¹¹ viral particles per injection).

### Immune Monitoring in Rhesus

For immune monitoring, 10-20 mL of blood was collected into vacutainer tubes containing heparin and maintained at room temperature until isolation. PBMCs were isolated by density gradient centrifugation using lymphocyte separation medium (LSM) and Leucosep separator tubes. PBMCs were stained with propidium iodide and viable cells counted using the Cytoflex LX (Beckman Coulter). Samples were then resuspended at 4×10⁶ cells/mL in RPMI complete (10% FBS).

### Splenocyte Isolation

For the evaluation of T-cell response, mouse spleens were extracted at various timepoints following immunization. Note that in some studies immunizations were staggered to enable spleens to be collected at the same time and compared. Spleens were collected and analyzed by IFNγ ELISpot and ICS. Spleens were suspended in RPMI complete (RPMI+10% FBS) and dissociated using the gentleMACS Dissociator (Miltenyi Biotec). Dissociated cells were filtered using a 40 μm strainer and red blood cells were lysed with ACK lysing buffer (150 mM NH₄Cl, 10 mM KHCO₃, 0.1 mM EDTA). Following lysis, cells were filtered with a 30 μm strainer and resuspended in RPMI complete.

### Serum Collection in Mice

At various timepoints post immunization 200 μL of blood was drawn. Blood was centrifuged at 1000 g for 10 minutes at room temperature. Serum was collected and frozen at -80° C.

### S1 IgG MSD/ELISA

96-well QuickPlex plates (Meso Scale Discovery, Rockville, MD) were coated with 50 μL of 1 μg/mL SARS-COV-2 SI (ACROBiosystems, Newark, DE), diluted in DPBS (Corning, Corning, NY), and incubated at 4° C. overnight. Wells were washed three times with agitation using 250 μL of PBS+0.05% Tween-20 (Teknova, Hollister, CA) and plates blocked with 150 μL Superblock PBS (Thermo Fisher Scientific, Waltham, MA) for 1 hour at room temperature on an orbital shaker. Test sera was diluted at appropriate series in 10% species-matched serum (Innovative Research, Novi, MI) and tested in single wells on each plate. Starting dilution 1:100, 3-fold dilutions, 11 dilutions per sample. Wells were washed and 50 μL of the diluted samples were added to wells and incubated for 1 hour at room temperature on an orbital shaker. Wells were washed and incubated with 25 μL of 1 μg/mL SULFO-TAG labeled anti-mouse antibody (MSD), diluted in DPBS+1% BSA (Sigma-Aldrich, St. Louis, MO), for 1 hour at room temperature on an orbital shaker. Wells were washed and 150 μL tripropylamine containing read buffer (MSD) added. Plates were run immediately using the QPlex SQ 120 (MSD) ECL plate reader. Endpoint titer is defined as the reciprocal dilution for each sample at which the signal is twice the background value, and is interpolated by fitting a line between the final two values that are greater than twice the background value. The background values is the average value (calculated for each plate) of the control wells containing 10% species-matched scrum only.

### Antibody Titers

For antibody response monitoring, antibody titers, including neutralizing antibody titers, in the sera were determined as described in J. Yu et al. (Science 10.1126/science. Abc6284, 2020), herein incorporated by reference for all purposes.

### IFNγ ELISpot Analysis

IFNγ ELISpot assays were performed using pre-coated 96-well plates (MAbtech, Mouse IFNγ ELISpot PLUS, ALP) following manufacturer's protocol. Samples were stimulated overnight with various overlapping peptide pools (15 amino acids in length, 11 amino acid overlap), at a final concentration of 1 μg/mL per peptide. For Spike—eight different overlapping peptide pools spanning the SARS-COV-2 Spike antigen (Genscript, 36-40 peptides per pool). Splenocytes were plated in duplicate at 1×10⁵ cells per well for each Spike pool, and 2.5×10⁴ cells per well (mixed with 7.5×10+naïve cells) for Spike pools 2, 4, and 7. To measure response to the TCE cassette—one pool spanning Nucleocapsid protein (JPT, NCap-1, 102 peptides), one spanning Membrane protein (JPT, VME-1, 53 peptides), and one spanning the Orf3a regions encoded in the cassette (Genscript, 38 peptides). For TCE peptide pools, splenocytes were plated in duplicate at 2×10⁵ cells per well for each pool. Sequences for peptide pools are presented in Table D (SEQ ID NOS. 27180-27495), Table E (SEQ ID NOS. 27496-27603), and Table F (SEQ ID NOS. 27604-27939). A DMSO only control was plated for each sample and cell number. Following overnight incubation at 37° C., plates were washed with PBS and incubated with anti-monkey IFNγ mAb biotin (MAbtech) for two hours, followed by an additional wash and incubation with Streptavidin-ALP (MAbtech) for one hour. After final wash, plates were incubated for ten minutes with BCIP/NBT (MAbtech) to develop the immunospots. Spots were imaged and enumerated using an AID reader (Autoimmun Diagnostika). For data processing and analysis, samples with replicate well variability (Variability=Variance/(median+1)) greater than 10 and median greater than 10 were excluded. Spot values were adjusted based on the well saturation according to the formula:

AdjustedSpots=RawSpots+2*(RawSpots*Saturation/(100−Saturation)

Each sample was background corrected by subtracting the average value of the negative control peptide wells. Data is presented as spot forming colonies (SFC) per 1×10⁶ splenocytes. Wells with well saturation values greater than 35% were labeled as “too numerous to count” (TNTC) and excluded. For samples and peptides that were TNTC, the value measured with 2.5×10⁴ cells/well was used.

### Vaccine Constructs

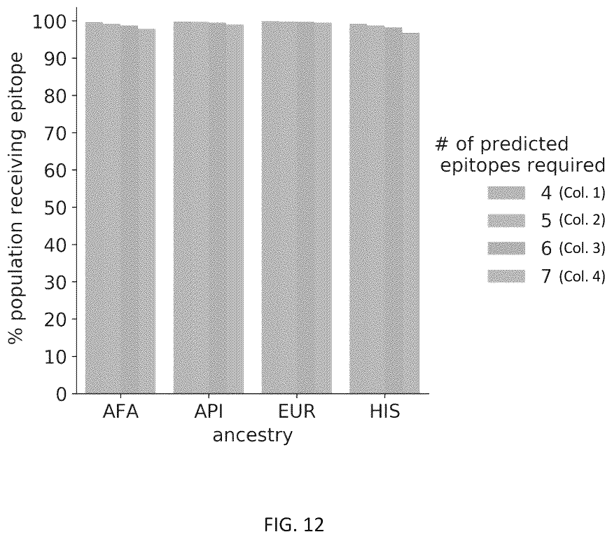

The various sequences evaluated are as follows:
“IDTSpike_{g}”: SARS-COV-2 Spike protein encoded by IDT optimized sequence (see SEQ ID NO:69) and including a D614G mutation with reference to SEQ ID NO:59 (see corresponding nucleotide mutation in SEQ ID NO:70); also referred to as “Spike V1”
“CTSpike_{g}”: SARS-COV-2 Spike protein encoded by Cool Tool optimized sequence version 1 (SEQ ID NO:79) including a D614G mutation with reference to SEQ ID NO:59 (see corresponding nucleotide mutation in SEQ ID NO:70); also referred to as “Spike V2.” In versions referred to as “CTSpike_{D}” D614 is not altered.
“CTSpikeF2P_{g}”: SARS-COV-2 Spike protein encoded by Cool Tool optimized sequence version 1 (SEQ ID NO:79) including a R682V to disrupt the Furin cleavage site (682-685 RRAR [SEQ ID NO: 125] to GSAS [SEQ ID NO: 126]); and K986P and V987P to interfere with the secondary structure of Spike with reference to the reference Spike protein (SEQ ID NO:59). The nucleotide sequence is shown in SEQ ID NO:89 and protein sequence shown in SEQ ID NO:90
“TCE5”: Selected CD8+epitopes predicted by the EDGE platform to be presented on MHC molecules for SARS-COV-2 proteins other than Spike. The 15 selected epitopes are presented along with their order in the cassette in Table 7. The nucleotide sequence is shown in SEQ ID NO:91 and protein sequence shown in SEQ ID NO:92. FIG. 12 shows the estimated protection across the four indicated populations for TCE5. All populations are estimated to have coverage above 95% up to at least the threshold of 7 epitopes (last column).
The SAM vector SAM-SGP1-TCE5-SGP2-CTSpikeGF2P is shown in SEQ ID NO:93
The ChAd vector ChAd-CMV-CTSpikeGF2P-CMV-TCE5 (EPE) is shown in SEQ ID NO:114
Additional vectors and Spike variants were designed for evaluation as shown in SEQ ID NOs: 109-113

TABLE 6
Encoded Spike Variants

CTSpikeF2Pg nucleotide (SEQ ID NO: 89); Bold Italic Furin Mutation 682-685 RRAR (SEQ ID
NO: 125) to GSAS (SEQ ID NO: 126), Bold Lower Case K986P and V987P
ATGTTTGTCTTCCTGGTCTTGCTGCCGCTGGTGAGCAGCCAGTGCGTGAATCTCACCACCCGCACCC
AGCTTCCACCTGCCTACACTAACAGCTTCACCCGAGGGGTGTATTACCCTGACAAGGTATTCCGGTC
CTCCGTCCTCCATAGCACGCAGGACCTTTTTCTGCCCTTCTTCTCAAATGTGACATGGTTCCATGCCA
TTCACGTGAGCGGCACGAATGGAACGAAGCGCTTTGATAACCCCGTGCTGCCTTTCAATGACGGCG
TCTACTTCGCCTCCACTGAAAAGTCAAACATCATCCGGGGCTGGATCTTTGGCACCACTCTTGATTC
AAAGACCCAGTCACTGCTGATTGTGAACAATGCTACAAACGTGGTTATCAAGGTGTGTGAGTTTCA
GTTCTGTAACGATCCATTTTTGGGAGTGTACTACCACAAGAACAACAAGTCCTGGATGGAGTCTGA
GTTCAGAGTGTATAGCTCTGCTAACAACTGCACCTTCGAGTACGTGTCCCAGCCTTTCCTTATGGAC
CTGGAAGGCAAACAGGGCAATTTCAAAAACCTGAGAGAGTTCGTGTTTAAGAACATTGACGGATA
CTTCAAAATTTATTCTAAGCACACACCAATTAACTTAGTGCGGGACCTACCCCAAGGCTTTAGCGCC
CTAGAGCCCCTGGTTGACCTGCCCATTGGGATCAATATAACAAGGTTCCAAACTCTACTGGCTCTGC
ATAGAAGTTATCTGACCCCAGGAGACAGCTCTAGTGGTTGGACCGCCGGCGCAGCAGCCTACTATG
TCGGGTACTTACAGCCACGCACGTTCCTTCTGAAGTACAATGAGAACGGGACAATCACTGACGCAG
TAGACTGTGCACTGGACCCGCTAAGCGAGACTAAGTGCACACTTAAATCCTTCACGGTGGAGAAAG
GCATTTATCAGACCTCTAACTTCAGGGTGCAGCCAACAGAAAGCATTGTGCGATTCCCAAATATTA
CTAATCTTTGCCCTTTCGGGGAGGTCTTTAATGCAACTAGATTCGCATCAGTCTATGCGTGGAACCG
CAAACGCATTTCCAATTGTGTCGCAGACTACTCAGTGCTGTACAACTCTGCCTCTTTCAGTACGTTC
AAGTGTTACGGAGTGTCACCCACTAAACTGAACGACCTGTGCTTTACAAATGTCTACGCTGACTCCT
TCGTGATTAGGGGAGACGAGGTGAGACAAATTGCCCCCGGACAGACTGGGAAGATTGCCGACTAC
AATTATAAGCTTCCTGATGATTTCACTGGCTGTGTTATTGCCTGGAATAGTAACAATCTGGATAGCA
AGGTGGGAGGCAACTATAACTACTTATATCGACTGTTTAGGAAGAGTAATCTGAAACCATTTGAGC
GGGATATTTCCACAGAAATTTACCAGGCCGGGAGCACACCATGTAATGGGGTGGAGGGATTTAATT
GTTACTTCCCACTCCAGAGCTATGGTTTCCAACCCACCAATGGAGTGGGTTACCAGCCCTATAGAGT
CGTGGTGCTTAGTTTTGAGCTGCTTCACGCCCCAGCAACCGTCTGCGGTCCCAAAAAGTCGACCAAT
CTCGTGAAAAACAAATGCGTAAACTTCAACTTTAACGGCTTAACAGGAACCGGCGTGCTCACCGAA
AGCAACAAGAAATTCCTTCCATTTCAGCAATTCGGAAGGGACATCGCCGACACAACAGACGCGGTG
AGGGACCCACAGACTCTGGAGATACTGGACATCACTCCTTGTTCGTTTGGGGGCGTCTCGGTCATC
ACACCCGGGACTAATACTAGTAATCAGGTAGCAGTTTTATATCAAGGCGTCAACTGTACCGAAGTA
CCTGTGGCCATACACGCTGATCAGCTAACGCCAACATGGCGAGTCTATTCCACCGGCTCTAACGTTT
TTCAGACCAGGGCTGGGTGCCTGATAGGGGCAGAGCACGTCAATAATTCCTATGAGTGTGATATCC
CCATAGGTGCGGGGATCTGTGCCAGCTATCAAACCCAAACCAATTCACCAgGGaGcGCAaGcTCTGT
GGCTTCTCAGAGCATAATTGCATATACAATGTCACTGGGCGCTGAGAATAGCGTTGCATACTCTAA
TAACAGCATAGCCATTCCCACGAACTTTACTATCAGTGTGACAACCGAAATATTGCCAGTTTCGATG
ACCAAAACTAGCGTGGATTGCACGATGTACATCTGTGGAGACTCTACCGAATGCAGCAATCTGCTA
TTACAATATGGCAGCTTCTGTACACAGTTAAATCGAGCCTTGACAGGCATCGCAGTGGAACAGGAC
AAAAATACTCAAGAGGTGTTTGCACAGGTGAAGCAAATCTACAAAACGCCCCCCATTAAAGATTTT
GGCGGGTTCAATTTTTCACAAATTCTCCCCGACCCGTCTAAGCCGAGTAAGCGGTCCTTCATCGAAG
ATCTGCTCTTTAACAAAGTAACCCTCGCCGATGCCGGCTTTATTAAGCAGTATGGCGACTGCCTGGG
GGATATAGCCGCTCGTGACCTGATTTGCGCCCAGAAGTTCAATGGTCTGACCGTGTTGCCTCCTTTA
TTGACCGATGAAATGATTGCCCAGTACACTAGTGCCCTGCTGGCCGGCACTATCACGTCTGGGTGG
ACCTTCGGAGCTGGTGCCGCCTTGCAGATACCTTTTGCAATGCAGATGGCCTATAGGTTTAATGGTA
TCGGAGTGACTCAGAACGTACTGTACGAGAACCAGAAGCTCATCGCTAATCAATTTAACTCCGCTA
TCGGAAAAATCCAGGACAGCCTCTCTTCTACAGCTAGCGCTCTGGGCAAACTGCAGGATGTCGTTA
ATCAGAATGCCCAGGCCCTGAACACCTTGGTTAAACAACTATCTTCCAACTTCGGGGCCATATCCA
GTGTGTTGAATGATATTCTCTCCCGCTTGGATccacctGAAGCTGAGGTGCAGATCGATCGCTTGATCA
CCGGCAGACTGCAGTCCCTCCAGACATATGTAACTCAGCAGCTGATTAGAGCCGCCGAGATAAGGG
CAAGTGCGAATCTGGCTGCCACCAAGATGAGCGAATGTGTATTGGGCCAGAGCAAACGAGTTGATT
TTTGCGGTAAGGGGTATCATTTAATGTCTTTCCCTCAATCCGCACCTCATGGCGTAGTTTTCCTGCAT
GTGACTTATGTCCCGGCTCAGGAGAAGAATTTTACCACAGCCCCCGCGATCTGCCATGACGGAAAG
GCCCACTTCCCCCGGGAAGGCGTGTTTGTATCCAATGGGACTCACTGGTTTGTCACTCAGCGAAATT
TTTATGAACCACAGATCATCACCACTGACAACACATTTGTTAGTGGAAACTGCGATGTGGTCATCG
GCATCGTGAATAACACTGTCTATGATCCACTGCAACCTGAACTGGATTCTTTTAAAGAGGAACTCG
ACAAGTATTTTAAAAACCACACTAGCCCTGACGTGGATCTCGGTGACATTTCTGGCATCAACGCTA
GCGTAGTGAACATTCAGAAAGAGATAGATAGACTTAATGAGGTGGCCAAGAACCTCAACGAAAGT
CTGATCGACCTCCAGGAACTGGGGAAATACGAGCAGTACATTAAATGGCCTTGGTACATATGGCTG
GGGTTCATTGCTGGGCTGATCGCAATAGTGATGGTGACCATAATGCTCTGTTGCATGACTAGCTGCT
GCAGCTGCCTGAAGGGCTGCTGTAGTTGTGGGTCATGTTGTAAGTTTGACGAAGATGATAGCGAGC
CTGTCCTTAAAGGAGTGAAGCTCCACTACACCTAG
CTSpikeF2Pg amino acid (SEQ ID NO: 90); Bold Italic Furin Mutation 682-685 RRAR (SEQ ID
NO: 125) to GSAS (SEQ ID NO: 126), Bold Lower Case K986P and V987P
MFVFLVLLPLVSSQCVNLTTRTQLPPAYTNSFTRGVYYPDKVFRSSVLHSTQDLFLPFFSNVTWFHAIHV
SGTNGTKRFDNPVLPFNDGVYFASTEKSNIIRGWIFGTTLDSKTQSLLIVNNATNVVIKVCEFQFCNDPFL
GVYYHKNNKSWMESEFRVYSSANNCTFEYVSQPFLMDLEGKQGNFKNLREFVFKNIDGYFKIYSKHTPI
NLVRDLPQGFSALEPLVDLPIGINITRFQTLLALHRSYLTPGDSSSGWTAGAAAYYVGYLQPRTFLLKYN
ENGTITDAVDCALDPLSETKCTLKSFTVEKGIYQTSNFRVQPTESIVRFPNITNLCPFGEVENATRFASVY
AWNRKRISNCVADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGSTPCNGVEGENCY
FPLQSYGFQPTNGVGYQPYRVVVLSFELLHAPATVCGPKKSTNLVKNKCVNFNFNGLTGTGVLTESNK
KFLPFQQFGRDIADTTDAVRDPQTLEILDITPCSFGGVSVITPGTNTSNQVAVLYQGVNCTEVPVAIHAD
QLTPTWRVYSTGSNVFQTRAGCLIGAEHVNNSYECDIPIGAGICASYQTQTNSPGSASSVASQSIIAYTMS
LGAENSVAYSNNSIAIPTNFTISVTTEILPVSMTKTSVDCTMYICGDSTECSNLLLQYGSFCTQLNRALTGI
AVEQDKNTQEVFAQVKQIYKTPPIKDFGGFNFSQILPDPSKPSKRSFIEDLLFNKVTLADAGFIKQYGDCL
GDIAARDLICAQKFNGLTVLPPLLTDEMIAQYTSALLAGTITSGWTFGAGAALQIPFAMQMAYRENGIG
VTQNVLYENQKLIANQFNSAIGKIQDSLSSTASALGKLQDVVNQNAQALNTLVKQLSSNFGAISSVLNDI
LSRLDppEAEVQIDRLITGRLQSLQTYVTQQLIRAAEIRASANLAATKMSECVLGQSKRVDFCGKGYHL
MSFPQSAPHGVVFLHVTYVPAQEKNFTTAPAICHDGKAHFPREGVFVSNGTHWFVTQRNFYEPQIITTD
NTFVSGNCDVVIGIVNNTVYDPLQPELDSFKEELDKYFKNHTSPDVDLGDISGINASVVNIQKEIDRLNE
VAKNLNESLIDLQELGKYEQYIKWPWYIWLGFIAGLIAIVMVTIMLCCMTSCCSCLKGCCSCGSCCKED
EDDSEPVLKGVKLHYT*

TABLE 7
TCE5 Cassette (Order of Frames as Shown)

| | | Start | End | SEQ | |
|---|---|---|---|---|---|
| Frame | Gene | aa | aa | ID NO | Amino Acid Sequence |
| 1 | ORF3a | 102 | 152 | 94 | EAPFLYLYALVYFLQSINFVRIIMRLWLCWKCRSKNPLL |
| | | | | | YDANYFLCWHTN |
| 2 | ORF3a | 53 | 85 | 95 | LAVFQSASKIITLKKRWQLALSKGVHFVCNLLL |
| 3 | ORF3a | 13 | 55 | 96 | VTLKQGEIKDATPSDFVRATATIPIQASLPFGWLIVGVAL |
| | | | | | LAV |
| 4 | N | 40 | 94 | 97 | RRPQGLPNNTASWFTALTQHGKEDLKFPRGQGVPINTN |
| | | | | | SSPDDQIGYYRRATRRI |
| 5 | M | 84 | 102 | 98 | MACLVGLMWLSYFIASFRL |
| 6 | N | 369 | 408 | 99 | KKDKKKKADETQALPQRQKKQQTVTLLPAADLDDESK |
| | | | | | QLQ |
| 7 | N | 284 | 338 | 100 | GNFGDQELIRQGTDYKHWPQIAQFAPSASAFFGMSRIG |
| | | | | | MEVTPSGTWLTYTGAIK |
| 8 | ORF3a | 169 | 198 | 101 | ITSGDGTTSPISEHDYQIGGYTEKWESGVK |
| 9 | N | 233 | 282 | 102 | KMSGKGQQQQGQTVTKKSAAEASKKPRQKRTATKAY |
| | | | | | NVTQAFGRRGPEQT |
| 10 | M | 56 | 75 | 103 | LLWPVTLACFVLAAVYRINW |
| 11 | N | 346 | 375 | 104 | FKDQVILLNKHIDAYKTFPPTEPKKDKKKK |
| 12 | N | 205 | 239 | 105 | TSPARMAGNGGDAALALLLLDRLNQLESKMSGKGQ |
| 13 | N | 100 | 118 | 106 | KMKDLSPRWYFYYLGTGPE |
| 14 | ORF3a | 199 | 254 | 107 | DCVVLHSYFTSDYYQLYSTQLSTDTGVEHVTFFIYNKIV |
| | | | | | DEPEEHVQIHTIDGSSG |
| 15 | N | 129 | 174 | 108 | GIIWVATEGALNTPKDHIGTRNPANNAAIVLQLPQGTTL |
| | | | | | PKGFYAE |

TCE5 Nucleotide Sequence (SEQ ID NO: 91):
ATGGCTGGCGAGGCCCCCTTCCTTTACCTGTACGCCCTTGTGTATTTCCTGCAGAGCATCAAT
TTTGTGAGAATCATCATGAGGCTGTGGCTTTGCTGGAAATGTAGGAGCAAGAACCCCCTGTT
GTATGACGCCAACTACTTTCTGTGTTGGCACACCAATCTCGCCGTGTTCCAGAGTGCCTCTA
AGATCATTACACTGAAAAAGCGGTGGCAGCTTGCACTTTCTAAGGGAGTGCATTTCGTTTGC
AACCTGCTCCTGGTGACACTCAAGCAGGGGGAAATCAAAGACGCCACCCCTAGCGACTTCG
TTAGAGCCACTGCCACAATCCCAATCCAGGCTTCCCTGCCTTTCGGCTGGCTTATCGTGGGT
GTGGCACTGTTGGCTGTGCGGAGACCACAGGGACTGCCTAATAATACAGCTAGCTGGTTTAC
CGCTCTGACACAGCATGGCAAAGAAGACCTCAAGTTCCCTCGCGGTCAGGGGGTGCCTATT
AACACTAATAGCTCTCCAGACGACCAAATTGGGTATTACAGGCGCGCCACAAGACGGATCA
TGGCCTGCTTAGTGGGGCTGATGTGGCTATCCTATTTTATTGCTAGCTTTCGCCTGAAGAAG
GACAAGAAGAAGAAAGCTGATGAGACCCAGGCACTGCCCCAGCGCCAAAAGAAGCAGCAG
ACAGTCACACTGCTCCCTGCTGCAGACCTGGATGACTTCAGCAAGCAGCTGCAGGGGAACT
TTGGCGACCAGGAGCTGATTAGACAGGGGACTGACTATAAGCATTGGCCTCAGATTGCTCA
GTTCGCCCCAAGTGCATCCGCCTTCTTCGGGATGTCACGAATAGGAATGGAAGTGACCCCTT
CTGGGACATGGTTGACATACACCGGAGCAATCAAGATTACCTCCGGGGACGGTACCACGTC
TCCTATTAGCGAACACGATTATCAGATAGGGGGATATACTGAGAAGTGGGAGTCCGGCGTC
AAAAAGATGAGTGGGAAAGGCCAGCAGCAACAAGGCCAAACAGTTACTAAAAAGTCTGCC
GCAGAAGCTAGTAAAAAGCCTCGCCAGAAGCGGACAGCCACCAAAGCTTACAATGTGACTC
AGGCCTTCGGCCGCCGGGGGCCTGAACAGACCCTCTTGTGGCCCGTTACCCTCGCATGTTTC
GTGCTTGCAGCTGTGTACAGGATCAATTGGTTTAAGGATCAGGTTATCCTGTTGAACAAACA
TATAGATGCCTATAAGACATTCCCACCCACCGAGCCAAAGAAAGATAAGAAAAAGAAAACT
AGTCCTGCAAGGATGGCCGGCAATGGAGGAGACGCAGCCTTAGCCCTGCTCTTACTCGACA
GGCTGAACCAACTTGAGTCTAAAATGAGCGGTAAAGGGCAGAAGATGAAGGATCTGTCCCC
AAGGTGGTATTTCTACTATCTGGGCACCGGCCCTGAGGATTGTGTCGTCCTCCACTCATACTT
CACTAGCGATTATTACCAGCTGTATAGTACACAATTATCTACCGACACAGGCGTCGAGCACG
TGACCTTCTTTATATACAATAAGATCGTGGATGAACCAGAGGAGCATGTGCAGATCCACACT
ATTGATGGCTCTAGCGGGGGCATCATCTGGGTGGCAACAGAAGGAGCCCTCAACACCCCAA
AGGACCATATCGGCACCAGGAATCCAGCCAACAATGCCGCCATTGTTCTGCAGCTCCCTCAG
GGCACTACTCTCCCTAAAGGCTTCTATGCTGAGGGACCCGGACCAGGCGCCAAATTTGTTGC
TGCTTGGACACTGAAAGCTGCTGCTGGGCCCGGACCAGGCCAGTACATCAAGGCCAACTCT
AAGTTTATCGGCATCACCGAATTGGGACCTGGACCCGGCTAG
TCE5 Amino Acid Sequence (SEQ ID NO: 92):
MAGEAPFLYLYALVYFLQSINFVRIIMRLWLCWKCRSKNPLLYDANYFLCWHTNLAVFQSASKI
ITLKKRWQLALSKGVHFVCNLLLVTLKQGEIKDATPSDFVRATATIPIQASLPFGWLIVGVALLA
VRRPQGLPNNTASWFTALTQHGKEDLKFPRGQGVPINTNSSPDDQIGYYRRATRRIMACLVGLM
WLSYFIASFRLKKDKKKKADETQALPQRQKKQQTVTLLPAADLDDFSKQLQGNFGDQELIRQG
TDYKHWPQIAQFAPSASAFFGMSRIGMEVTPSGTWLTYTGAIKITSGDGTTSPISEHDYQIGGYTE
KWESGVKKMSGKGQQQQGQTVTKKSAAEASKKPRQKRTATKAYNVTQAFGRRGPEQTLLWP
VTLACFVLAAVYRINWFKDQVILLNKHIDAYKTFPPTEPKKDKKKKTSPARMAGNGGDAALAL
LLLDRLNQLESKMSGKGQKMKDLSPRWYFYYLGTGPEDCVVLHSYFTSDYYQLYSTQLSTDTG
VEHVTFFIYNKIVDEPEEHVQIHTIDGSSGGIIWVATEGALNTPKDHIGTRNPANNAAIVLQLPQG
TTLPKGFYAEGPGPGAKFVAAWTLKAAAGPGPGQYIKANSKFIGITELGPGPG-
SAM-SGP1-TCE5-SGP2-CTSpike_{G}F2P Nucleotide Sequence (SEQ ID NO: 93): (NT 1-17:
T7 promoter; NT 62-7543: VEEV non-structural protein coding region; NT 7518-7560: SGP1; NT
7582-7587 and 9541-9546: Kozak sequence; NT 7588-9474: TCE5 cassette; NT 9480-9540: SGP2; NT
9547-13368: CTSpike Furin-2P); See Sequence Listing.
ChAd-CMV-CTSpike_{G}F2P-CMV-TCE5 (EPE) Nucleotide Sequence (SEQ ID NO: 114);
See Sequence Listing.

### XIV.E. SARS-COV-2 Vaccine Produces Responses to Various Spike Constructs

ChAd and SAM vaccine platforms encoding various versions of the SARS-COV-2 Spike protein were assessed.

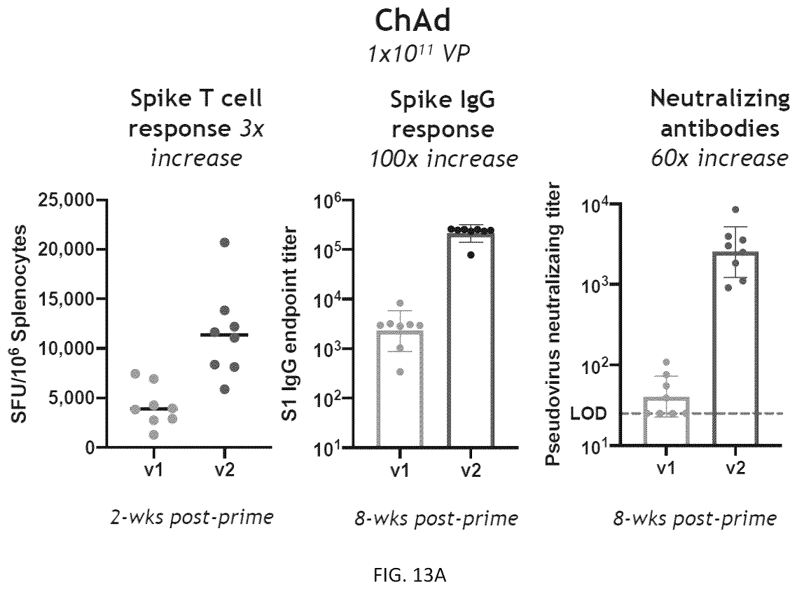

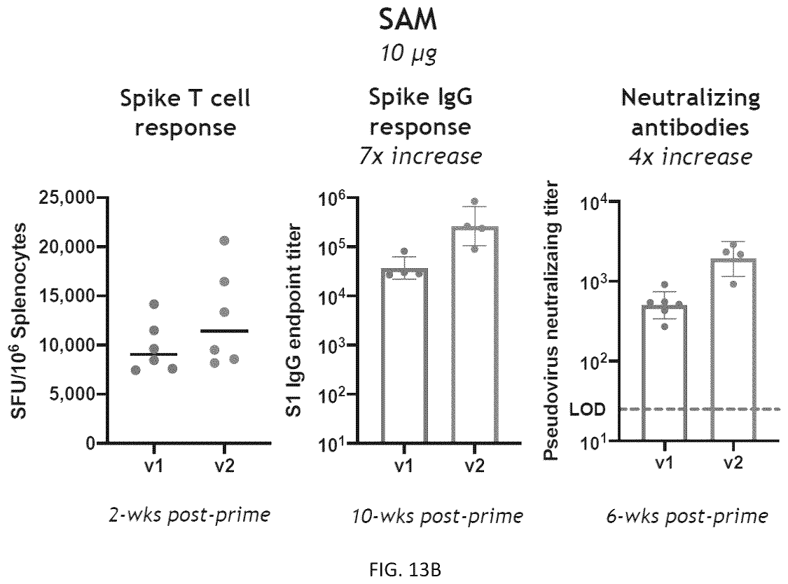

Versions of a Spike-encoding cassette featuring different sequence optimizations were assessed: “IDTSpike_{g}” (SEQ ID NO:69, also referred to as “Spike V1” or “v1”); “CTSpike_{g}”: (SEQ ID NO:79, also referred to as “Spike V2” or “v2”). As shown in FIG. 13, both ChAd (FIG. 13A) and SAM (FIG. 13B) vaccines produced detectable T cell responses (left panel), Spike-specific IgG antibodies (middle panel) and neutralizing antibodies (right panel). Notably, a ChAd vaccine encoding the CTSpike_{g} sequence version produced a 3-fold increased T cell response, 100-fold increased IgG production, and 60-fold increase in neutralizing antibody titer. Similarly, a SAM vaccine encoding the CTSpike_{g} sequence version produced an increased T cell response, 7-fold increase in IgG production, and 4-fold increase in neutralizing antibody titer. Accordingly, the data demonstrate sequence optimization of the Spike cassette produced an increased immune response across the multiple parameters assessed for each vaccine platform examined.

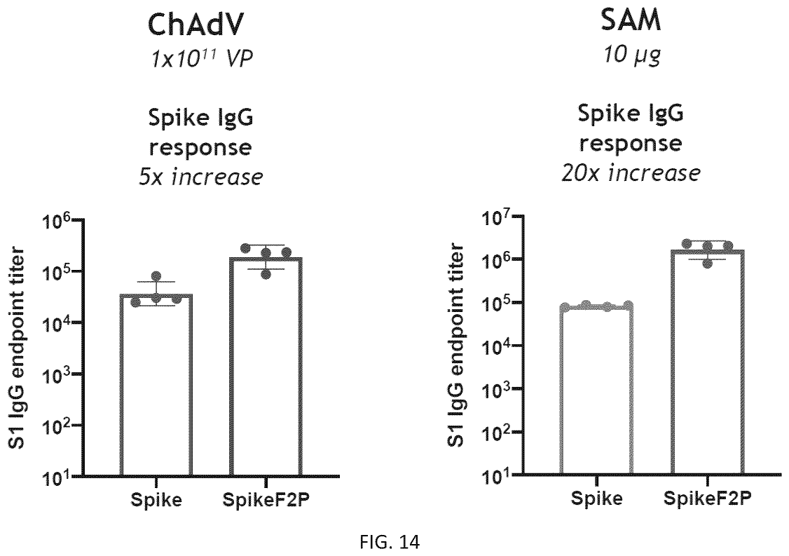

A version of a Spike-encoding cassette featuring modified Spike that includes removal of a furin site and addition of prolines in S2 domain was assessed: “CTSpikeF2P_{g}” (SEQ ID NO:89 and SEQ ID NO:90). As shown in FIG. 14, both ChAd (left panel) and SAM (right panel) vaccines encoding the F2P-modified Spike produced 5-fold and 20-fold Spike-specific IgG antibodies, respectively, relative to a corresponding “CTSpike_{g}” cassette that does not have the referenced modifications. Accordingly, the data demonstrate modification of the Spike cassette produced an increased antibody response for each vaccine platform examined.

### XIV.F. SARS-COV-2 Vaccine Produces Responses to T Cell Epitopes

ChAd and SAM vaccine platforms encoding various a modified SARS-COV-2 Spike protein and a T cell epitope (TCE) cassette encoding EDGE predicted epitopes (EPE) were assessed.

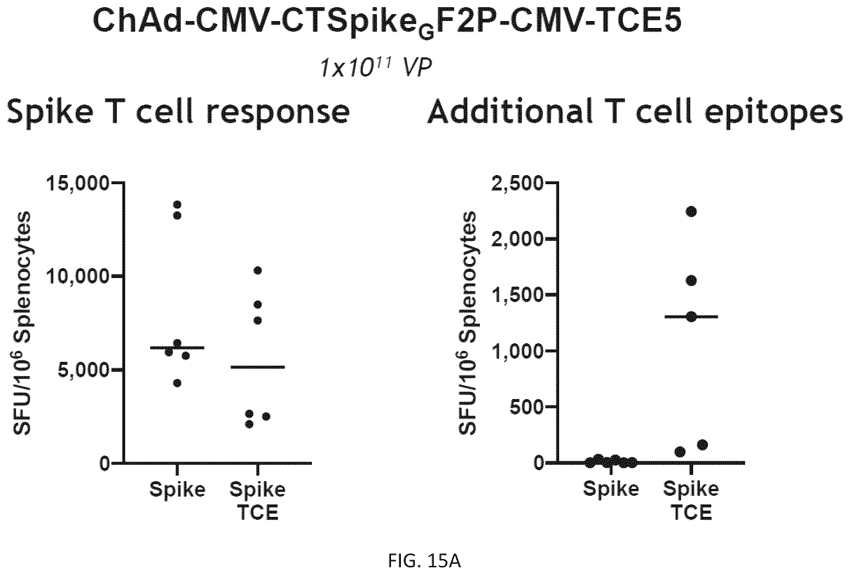

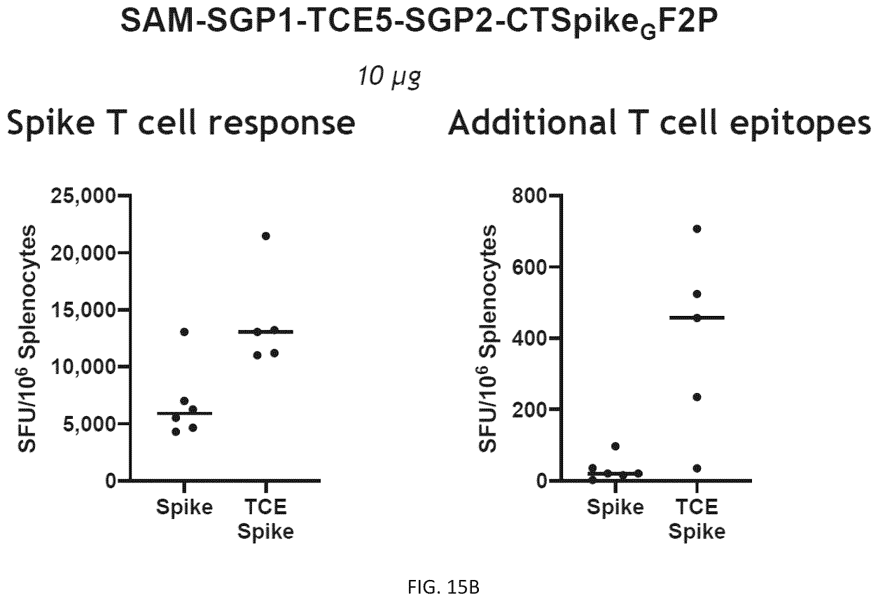

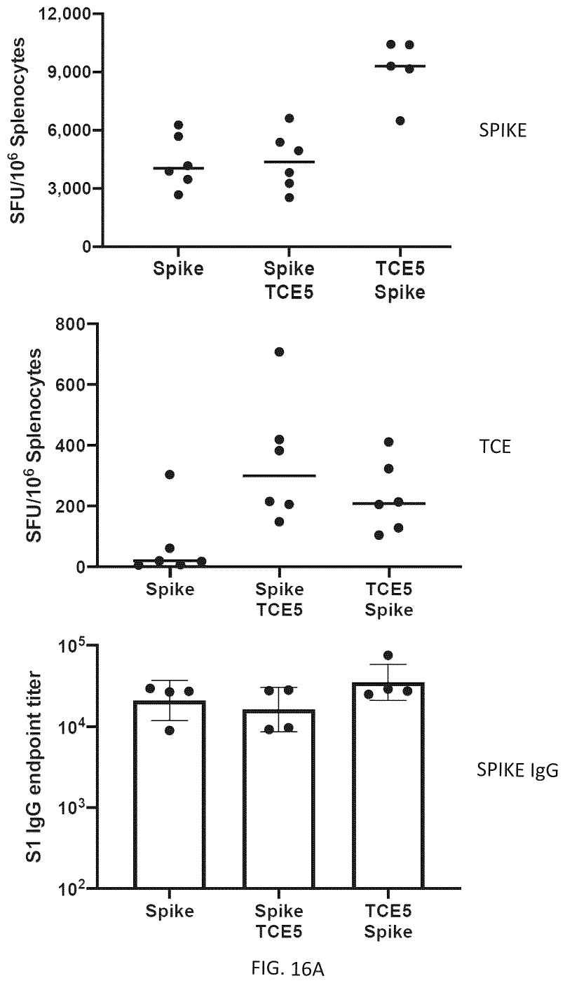

Both a modified Spike-encoding only cassette (“CTSpikeF2P_{g}” (SEQ ID NO:89) and modified Spike together with additional non-Spike T cell epitopes (ChAd SEQ ID NO:114; SAM SEQ ID NO:93; see Table 7 for “TCE5”), and immune responses assessed, as described above. As shown in FIG. 15, both ChAd (FIG. 15A) and SAM (FIG. 15B) each vaccine assessed produced detectable T cell responses to Spike (left panel), while vaccines including the TCE5 cassette also generally produced detectable T cell responses to the encoded T cell epitopes (right panel). Accordingly, the data demonstrate addition of a T cell epitope cassette led to broad T cell responses across the SARS-COV-2 Genome for each vaccine platform examined.

### XIV.G. Order of Cassettes in SARS-COV-2 Vaccine Influences Immune Response

SAM vaccine platforms encoding various orders of a modified SARS-COV-2 Spike protein and a T cell epitope (TCE) cassette encoding EDGE predicted epitopes (EPE) were assessed.

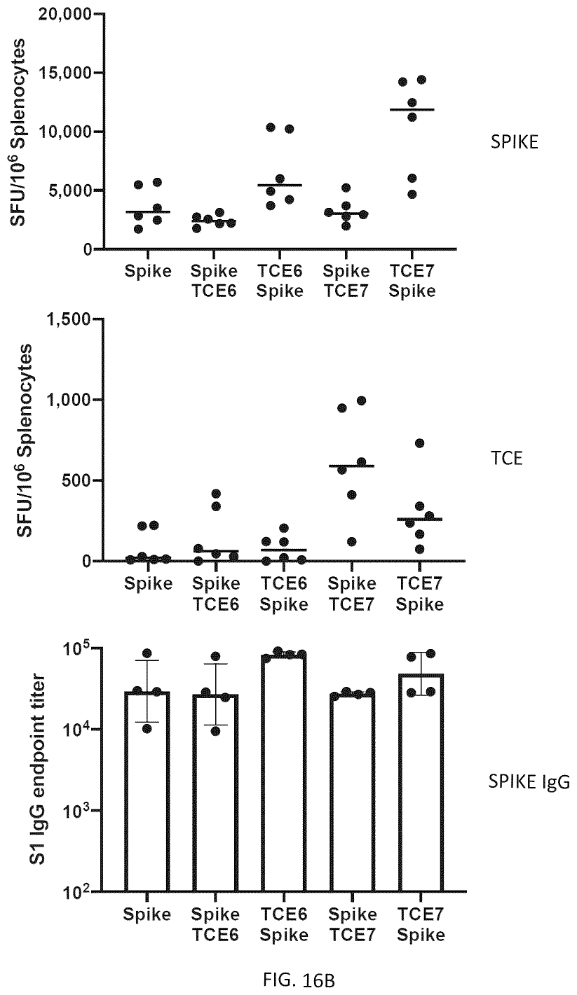

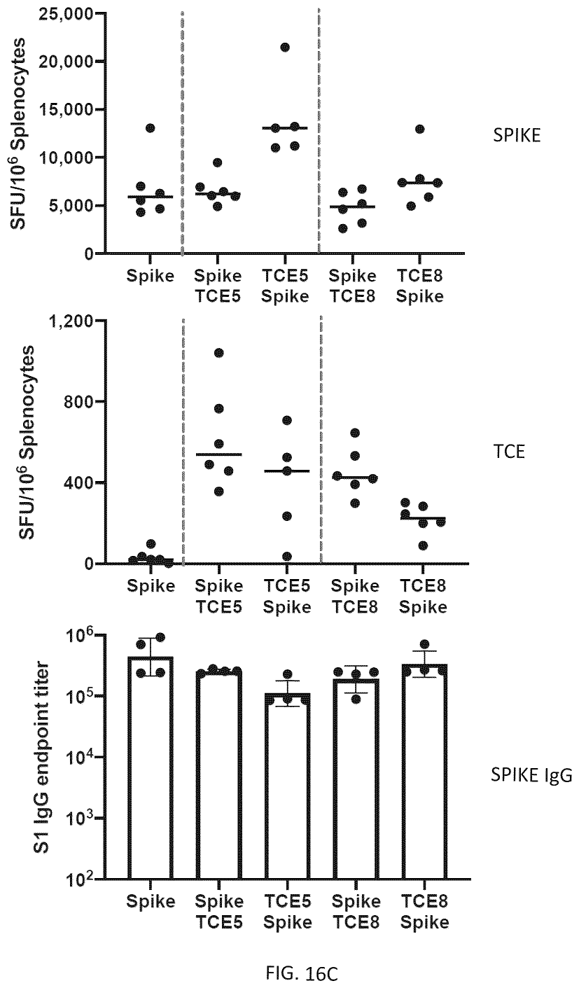

As shown in FIG. 16, T cell responses to Spike (top panel), T cell responses to the encoded T cell epitopes (middle panel), and Spike-specific IgG antibodies (bottom panel) were produced when vaccinated with various constructs. In FIG. 16A, SAM constructs included “IDTSpike_{g}” (SEQ ID NO:69) alone (left columns), IDTSpike_{g} expressed from a first subgenomic promoter followed by TCE5 expressed from a second subgenomic promoter (middle columns), or TCE5 expressed from a first subgenomic promoter followed by IDTSpike_{g} expressed from a second subgenomic promoter (right columns), with immune responses assessed, as described above. In FIG. 16B, SAM constructs included “IDTSpike_{g}” (SEQ ID NO:69) alone (first column), IDTSpike_{g} expressed from a first subgenomic promoter followed by TCE6 or TCE7 expressed from a second subgenomic promoter (columns 2 and 4, respectively), or TCE6 or TCE7 expressed from a first subgenomic promoter followed by IDTSpike_{g} expressed from a second subgenomic promoter (columns 3 and 5, respectively), with immune responses assessed, as described above. In FIG. 16C, SAM constructs included “CTSpike_{g}” (SEQ ID NO:79) alone (first column), CTSpike_{g} expressed from a first subgenomic promoter followed by TCE5 or TCE8 expressed from a second subgenomic promoter (columns 2 and 4, respectively), or TCE5 or TCE8 expressed from a first subgenomic promoter followed by CTSpike_{g} expressed from a second subgenomic promoter (columns 3 and 5, respectively), with immune responses assessed, as described above. Generally, and in particular for Spike, T cell responses were increased when the respective epitopes were expressed from the second subgenomic promoter, including increased Spike-directed T cell responses relative to Spike alone. A similar trend was also observed generally observed with increased Spike-specific IgG titers when the Spike antigen was expressed from the second subgenomic promoter except, for potentially the CTSpike_{g} constructs. Accordingly, the data demonstrate sequence order of antigen cassettes in vaccine platforms influenced immune responses.

### XIV.H. Additional SARS-COV-2 Vaccine Constructions

Vaccines were constructed to maximize the percentage of people getting predicted to achieve a total magnitude of greater than 1000 from validated epitopes. Briefly, magnitude was calculated across all validated epitopes in the starting proteins (e.g., Spike), as well as any added in the TCE cassettes according to the following with an expected approximate upper size limit of 600 amino acids beyond that of Spike: (1) An individual's magnitude is the sum of all epitope magnitudes across their respective diplotype alleles; (2) Each epitopes magnitude=(magnitude of response)×(Frequency of positive response/100), with values found in Tarke et al. (Comprehensive analysis of T cell immunodominance and immunoprevalence of SARS-COV-2 epitopes in COVID-19 cases. Cell Rep Med. 2021 Feb. 16; 2(2):100204. doi: 10.1016/j.xcrm.2021.100204. Epub 2021 Jan. 26.), herein incorporated by reference for all purposes; (3) epitopes other than those from starting proteins that span mutations with >5% frequency were excluded (see Table 8 for all mutations >1% frequency either overall or in specific strains), though mutations are allowed in flanking regions; and (4) cassette order was chosen to minimize unintended junction epitopes across adjacent frames, as well as minimize consecutive frames in the same protein to reduce chance of functional protein fragments, as described above.

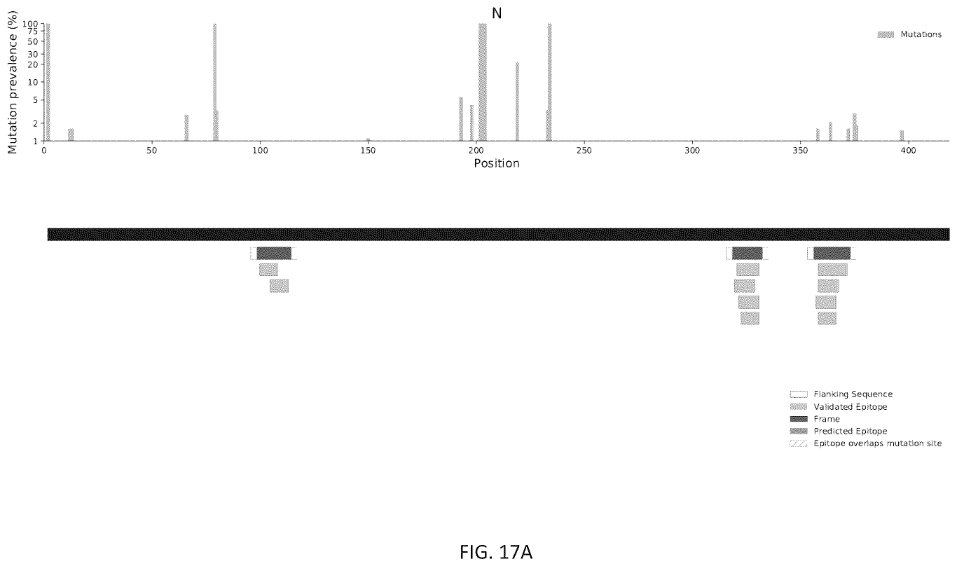

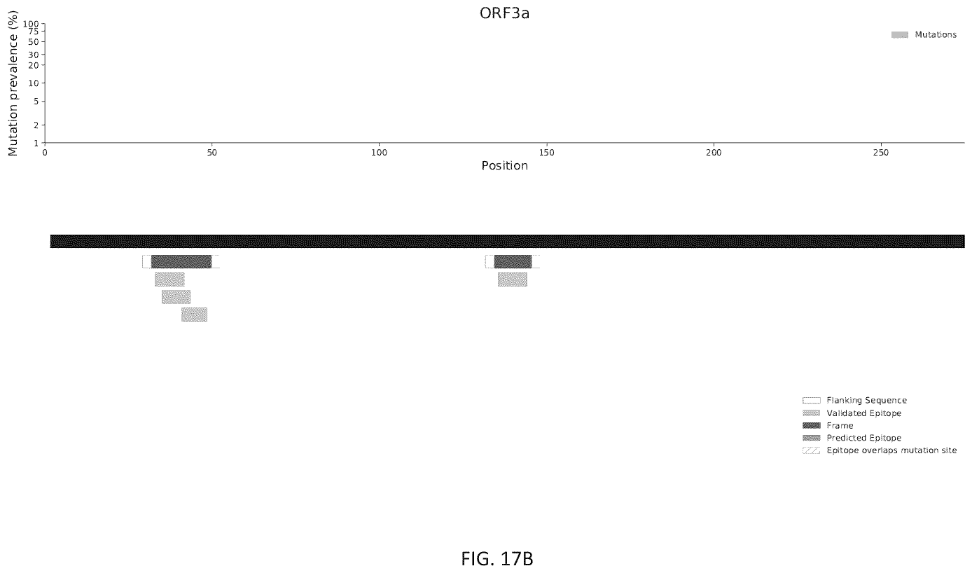

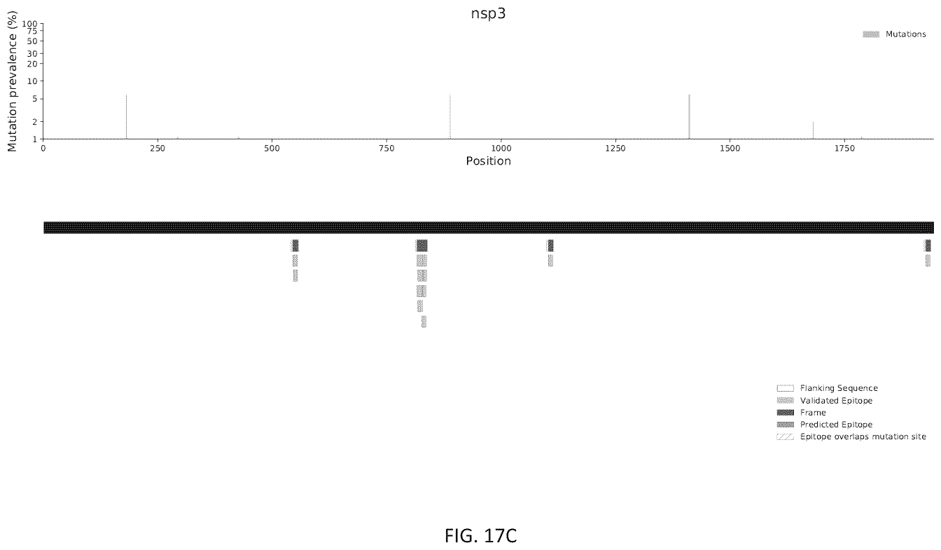

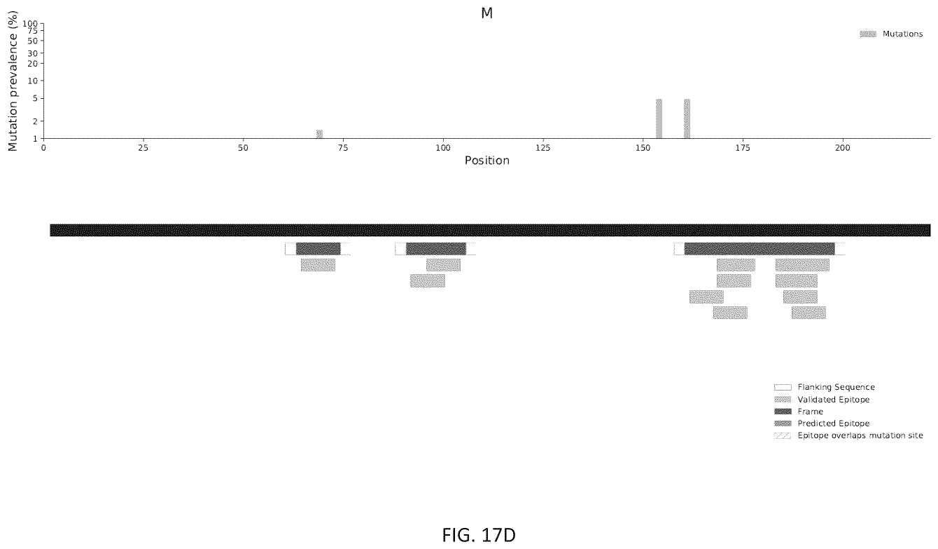

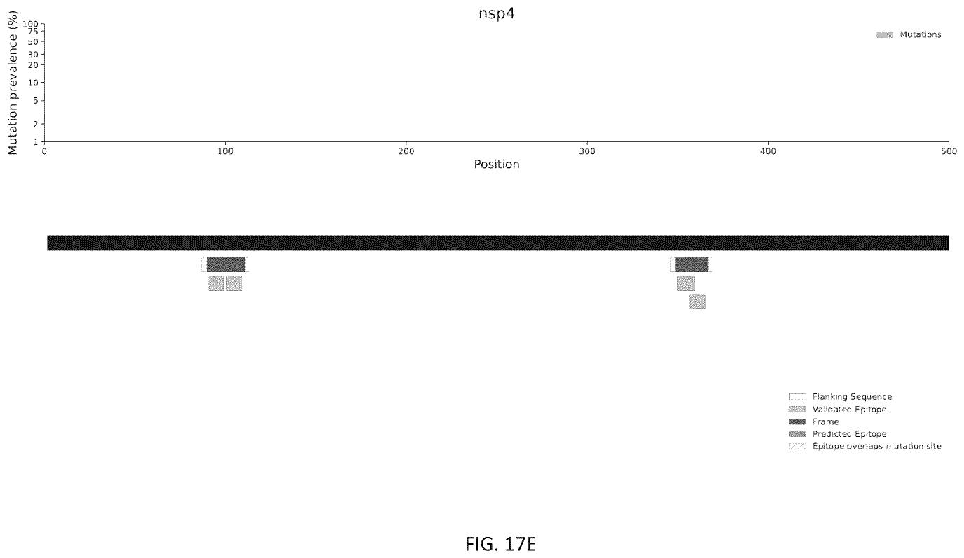

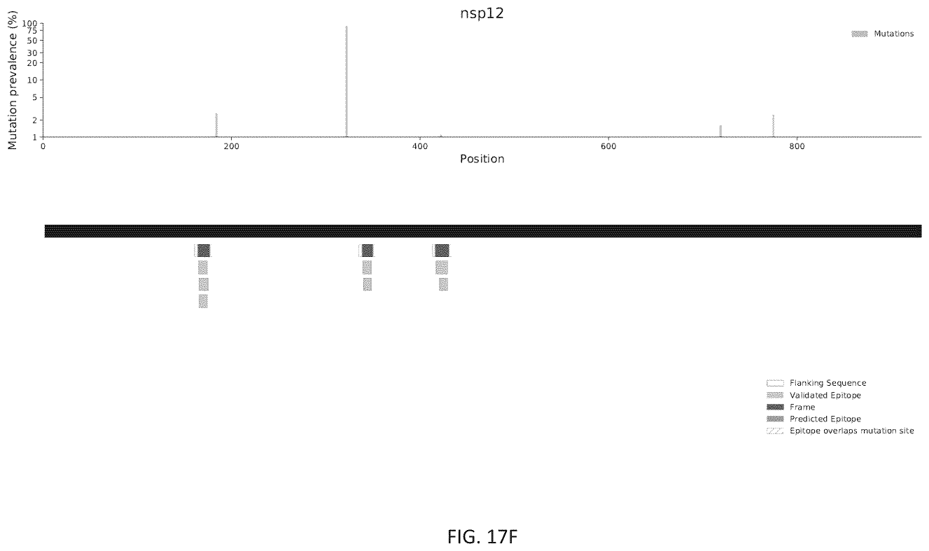

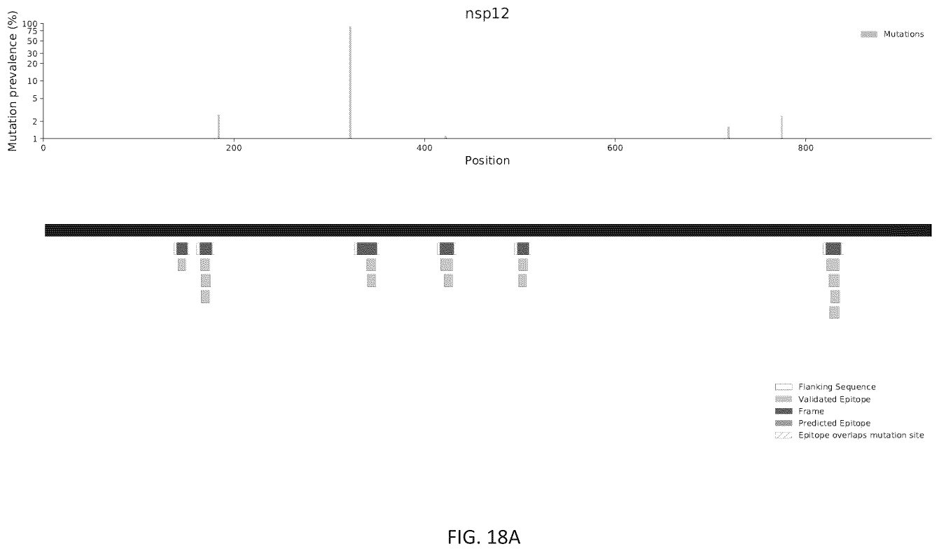

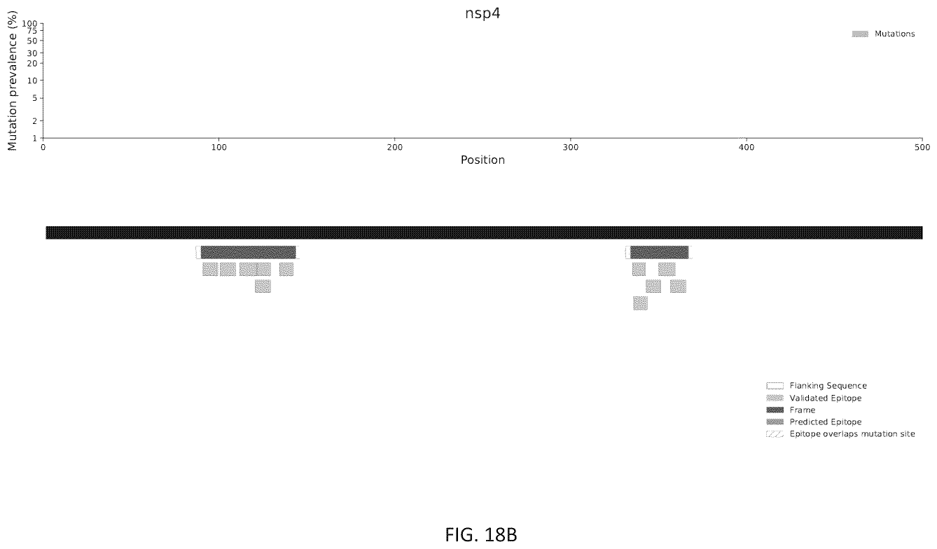

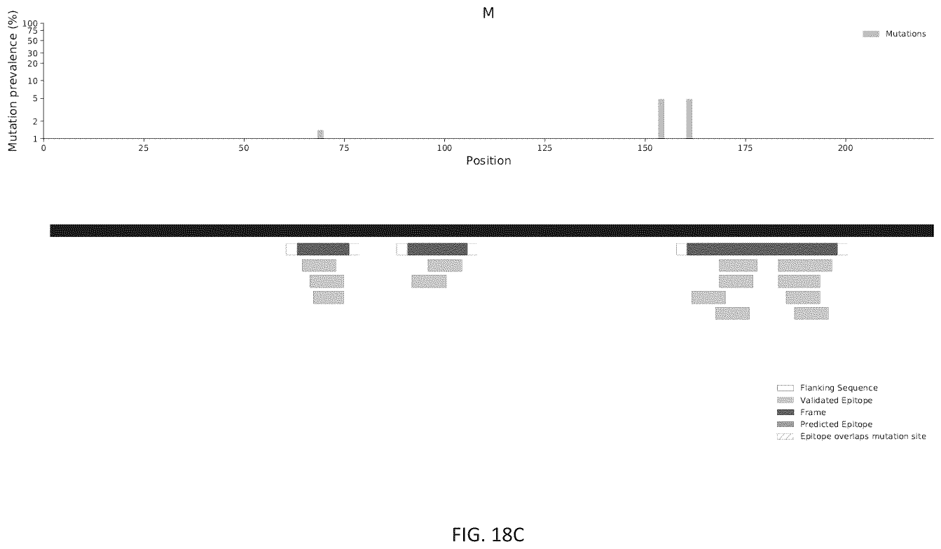

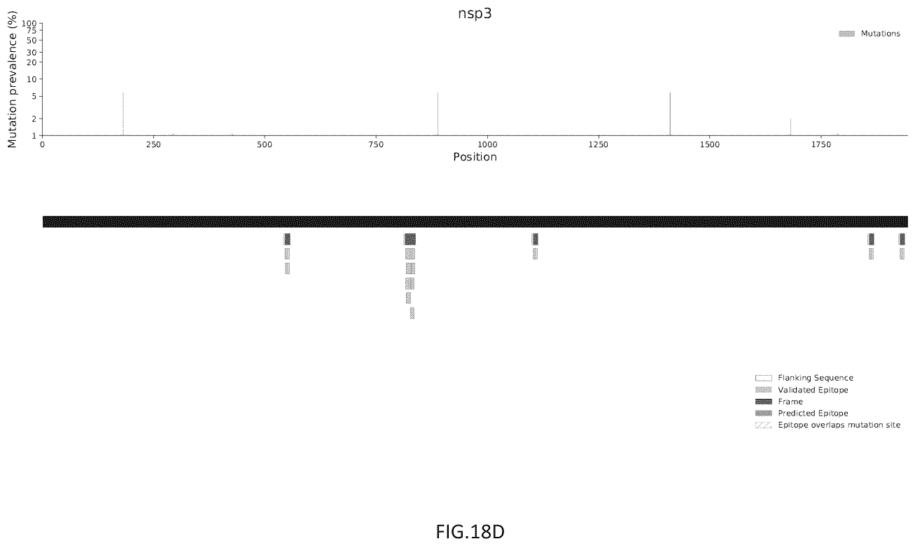

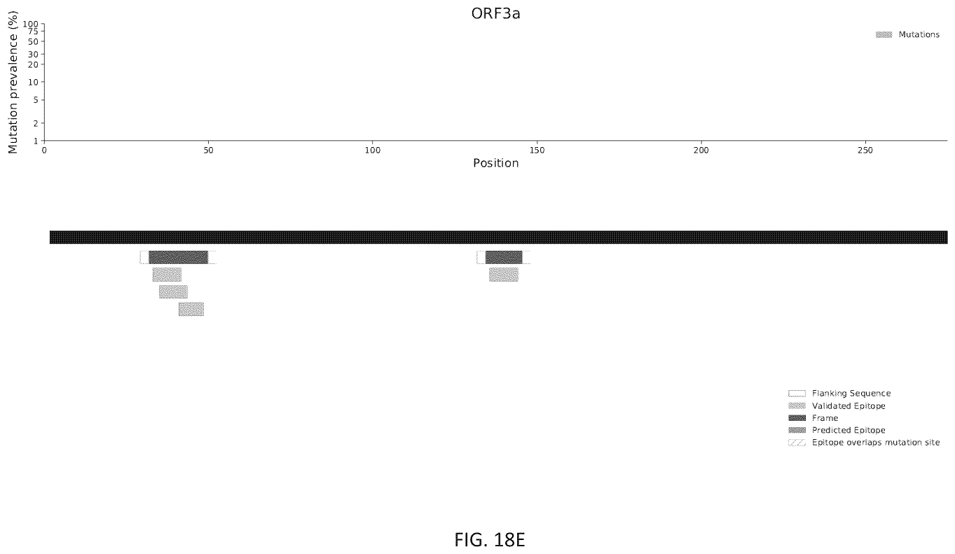

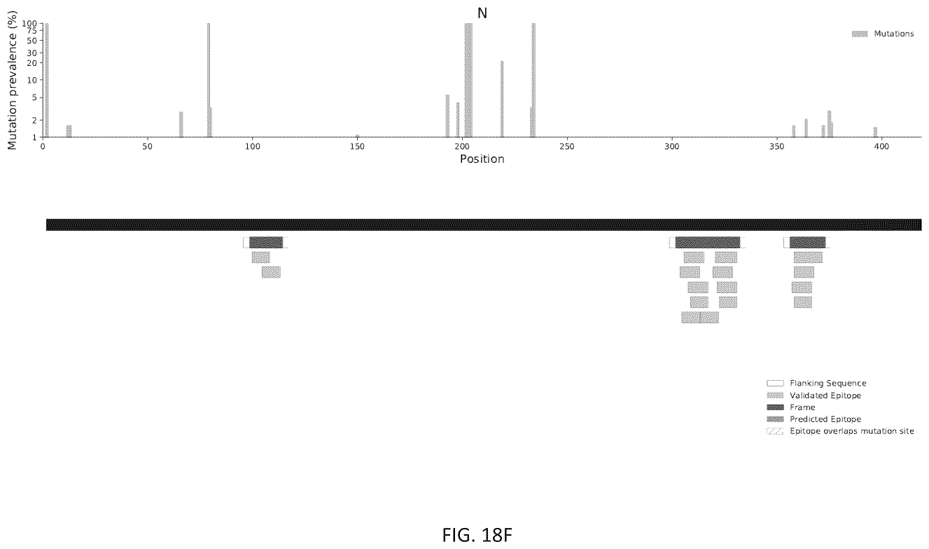

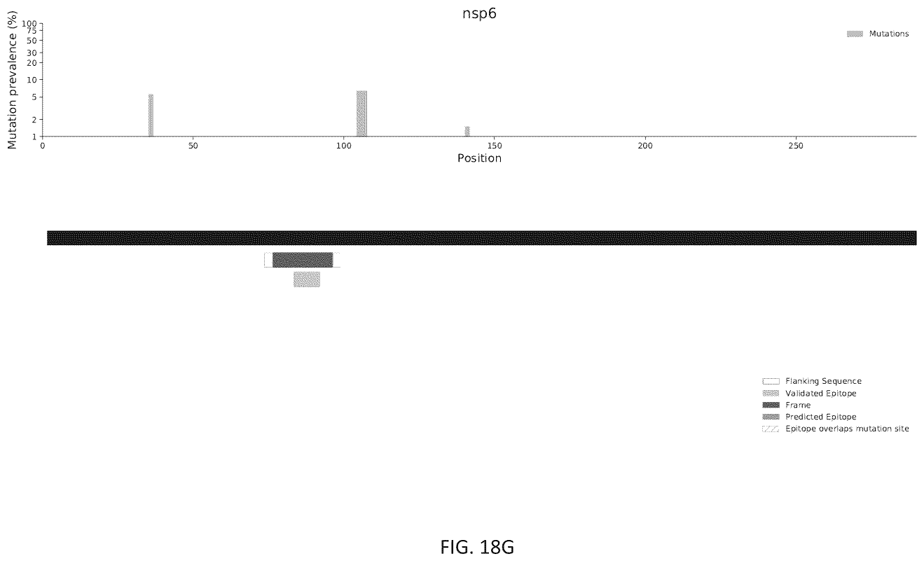

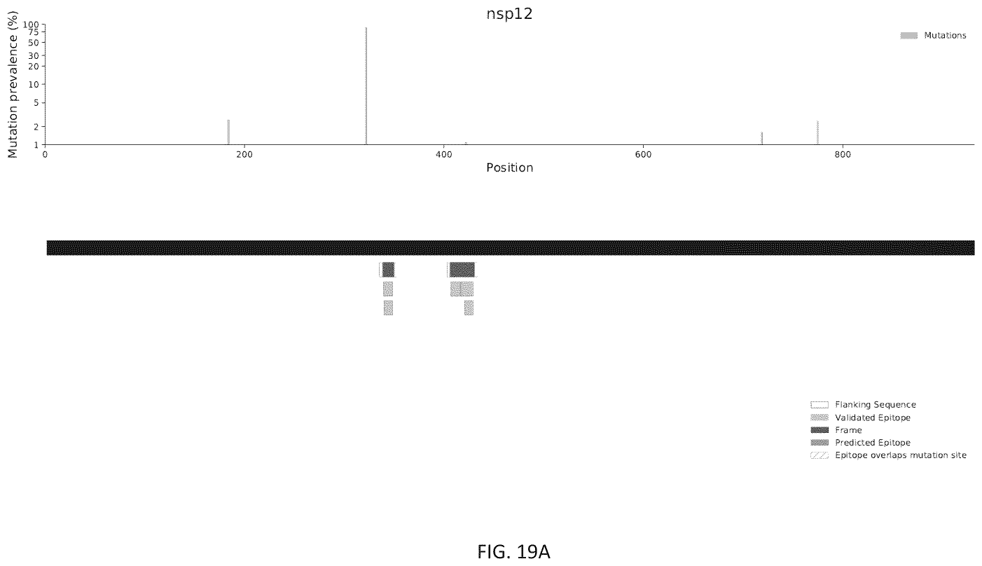

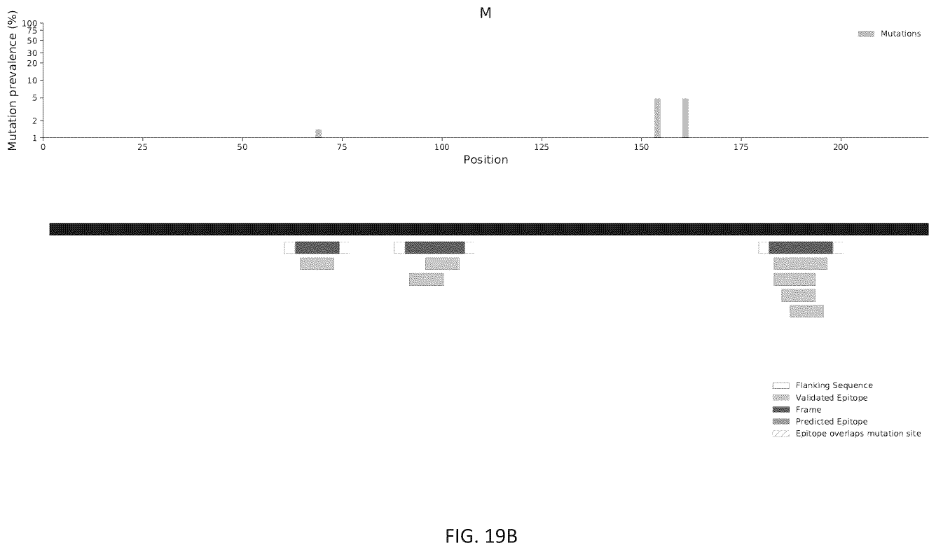

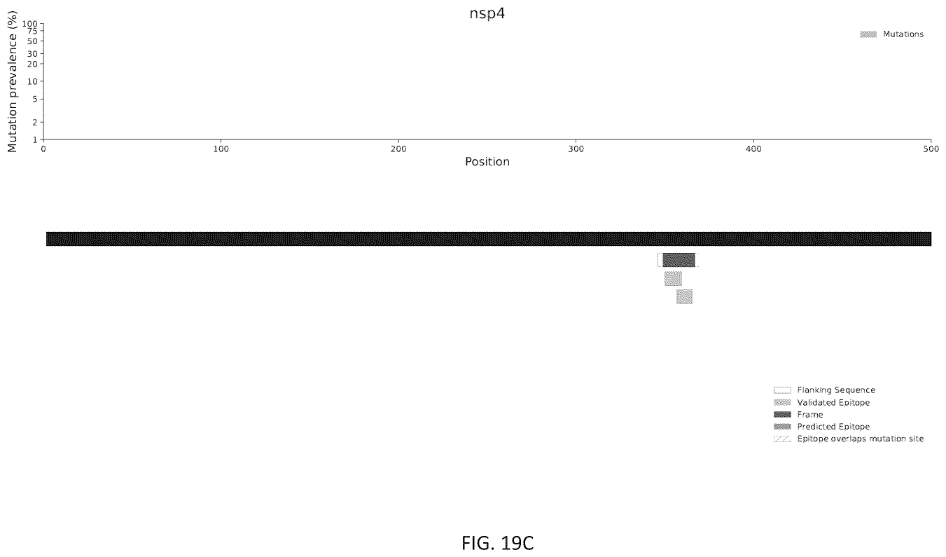

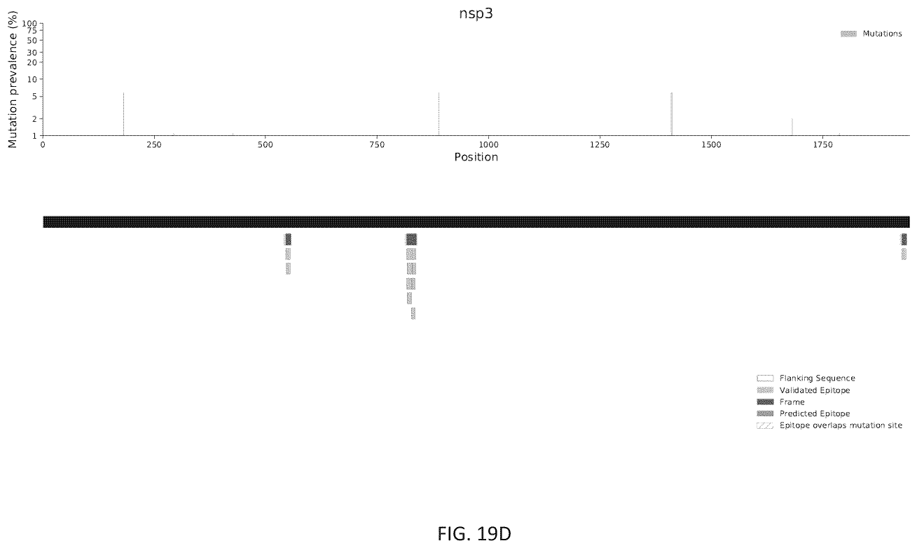

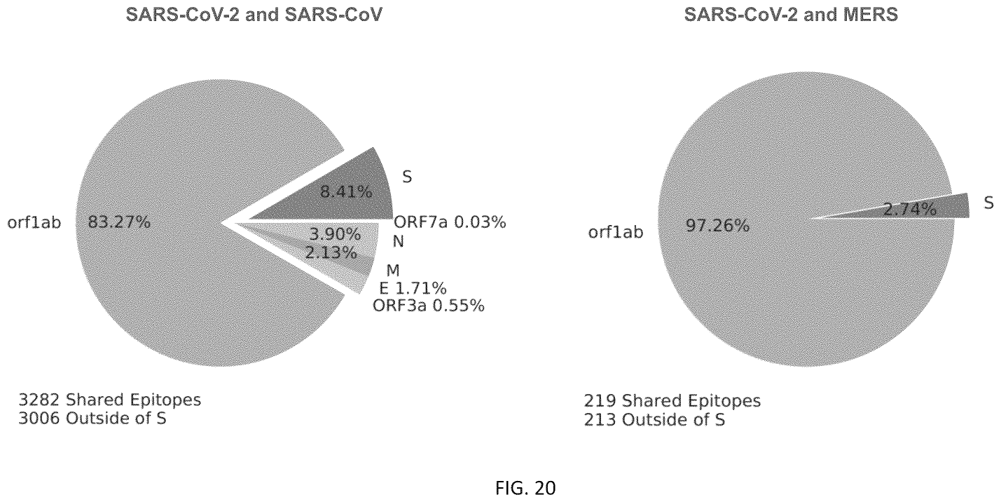

The following constructions were produced: (A) “TCE10” starting with full Spike protein as the starting point and adding validated epitopes according to the above for a total size of 378 amino acids in addition to Spike (Table 9A, maps of epitopes covered in FIG. 17A-17F); (B) “TCE9” extended TCE10 and adding validated epitopes according to the above only if fully conserved between SARS and SARS-2 (e.g., as a pan-coronavirus vaccine), with certain frames extended (21 additional amino acids across all frames) to include additional predicted epitopes for alleles (i.e., not validated epitopes), for a total size of 556 amino acids in addition to Spike (Table 9B, maps of epitopes covered in FIG. 18A-18G); (C) “TCE11” starting with full Spike and Nucleocapsid proteins as the starting point and adding validated epitopes according to the above for a total size of 616 amino acids (197aa+full N) in addition to Spike (Table 9C, maps of epitopes covered in FIG. 19A-19D). Table 10A and Table 10B presents the magnitude coverage across various populations for each of TCE5, TCE9, TCE10, and TCE11 for SARS-CoV-2 and SARS/SARS-COV-2 conserved epitopes, respectively. Notably, each of the vaccine constructs cover greater than 89% of each of the indicated populations with a validated response magnitude greater than 1000 and greater than 95% with a validated response magnitude greater than 100, while TCE9 covers greater than 74% of each of the indicated populations with a validated response magnitude greater than 1000 for epitopes conserved between SARS and SARS-2. FIG. 20 presents the percentages of shared candidate 9-mer epitope distribution between SARS-COV-2 and SARS-COV (left panel) and between SARS-COV-2 and MERS (right panel), highlighting the significant number of conserved sequences outside of the Spike protein demonstrating the value of evaluating and including epitopes beyond those simply encoded by Spike, particularly with a goal of constructing a pan-coronavirus vaccine.

TABLE 8
Mutations >1% frequency either overall or in specific strains

| Gene | Position | Strain | Frequency |
|---|---|---|---|
| M | 70 | b117 | 0.014 |
| M | 155 | N501Y_early | 0.048 |
| M | 162 | N501Y_early | 0.048 |
| N | 3 | b117 | 0.999 |
| N | 13 | N501Y_early | 0.016 |
| N | 14 | N501Y_early | 0.016 |
| N | 67 | | 0.028 |
| N | 80 | P1 | 1 |
| N | 81 | N501Y_early | 0.033 |
| N | 151 | | 0.011 |
| N | 194 | | 0.056 |
| N | 199 | | 0.041 |
| N | 203 | b117 | 1 |
| N | 204 | b117 | 1 |
| N | 205 | N501Y_early | 1 |
| N | 220 | | 0.218 |
| N | 234 | | 0.033 |
| N | 235 | b117 | 0.999 |
| N | 359 | N501Y_early | 0.016 |
| N | 365 | | 0.021 |
| N | 373 | N501Y_early | 0.016 |
| N | 376 | | 0.029 |
| N | 377 | | 0.018 |
| N | 398 | | 0.015 |
| ORF3a | 15 | b117 | 0.089 |
| ORF3a | 38 | | 0.011 |
| ORF3a | 57 | N501Y_early | 0.982 |
| ORF3a | 90 | b117 | 0.02 |
| ORF3a | 131 | N501Y_early | 0.036 |
| ORF3a | 171 | N501Y_early | 0.589 |
| ORF3a | 172 | | 0.039 |
| ORF3a | 202 | | 0.012 |
| ORF3a | 223 | | 0.016 |
| ORF3a | 251 | | 0.017 |
| ORF3a | 259 | N501Y_early | 0.018 |
| S | 5 | | 0.012 |
| S | 18 | N501Y_early | 0.381 |
| S | 54 | N501Y_early | 0.016 |
| S | 69 | b117 | 1 |
| S | 70 | b117 | 1 |
| S | 80 | N501Y_early | 1 |
| S | 98 | | 0.012 |
| S | 144 | b117 | 1 |
| S | 215 | N501Y_early | 1 |
| S | 222 | | 0.198 |
| S | 241 | N501Y_early | 1 |
| S | 242 | N501Y_early | 1 |
| S | 243 | N501Y_early | 1 |
| S | 246 | N501Y_early | 0.016 |
| S | 262 | | 0.011 |
| S | 316 | N501Y_early | 0.016 |
| S | 384 | N501Y_early | 0.016 |
| S | 417 | N501Y_early | 0.984 |
| S | 439 | | 0.017 |
| S | 477 | | 0.048 |
| S | 484 | N501Y_early | 1 |
| S | 501 | b117 | 1 |
| S | 570 | b117 | 1 |
| S | 614 | b117 | 1 |
| S | 681 | b117 | 1 |
| S | 682 | cleavage | 1 |
| S | 683 | cleavage | 1 |
| S | 685 | cleavage | 1 |
| S | 688 | N501Y_early | 0.016 |
| S | 701 | N501Y_early | 1 |
| S | 706 | b117 | 0.018 |
| S | 716 | b117 | 1 |
| S | 982 | b117 | 1 |
| S | 1078 | N501Y_early | 0.016 |
| S | 1117 | N501Y_early | 0.016 |
| S | 1118 | b117 | 1 |
| S | 1150 | N501Y_early | 0.016 |
| nsp12 | 4577 | | 0.026 |
| nsp12 | 4646 | | 0.01 |
| nsp12 | 4715 | | 0.885 |
| nsp12 | 4815 | | 0.011 |
| nsp12 | 5112 | | 0.016 |
| nsp12 | 5168 | | 0.025 |
| nsp13 | 5542 | | 0.025 |
| nsp13 | 5585 | | 0.026 |
| nsp13 | 5614 | | 0.022 |
| nsp13 | 5784 | | 0.027 |
| nsp13 | 5922 | | 0.017 |
| nsp14 | 6054 | | 0.024 |
| nsp14 | 6426 | | 0.011 |
| nsp15 | 6485 | | 0.015 |
| nsp16 | 7014 | | 0.028 |
| nsp2 | 265 | | 0.132 |
| nsp2 | 300 | | 0.042 |
| nsp3 | 1001 | | 0.059 |
| nsp3 | 1113 | | 0.011 |
| nsp3 | 1246 | | 0.011 |
| nsp3 | 1361 | | 0.01 |
| nsp3 | 1708 | | 0.058 |
| nsp3 | 2230 | | 0.058 |
| nsp3 | 2501 | | 0.02 |
| nsp3 | 2606 | | 0.011 |
| nsp4 | 3087 | | 0.025 |
| nsp5 | 3278 | | 0.016 |
| nsp5 | 3352 | | 0.034 |
| nsp5 | 3353 | | 0.013 |
| nsp5 | 3371 | | 0.013 |
| nsp6 | 3606 | | 0.056 |
| nsp6 | 3675 | | 0.064 |
| nsp6 | 3676 | | 0.064 |
| nsp6 | 3677 | | 0.064 |
| nsp6 | 3711 | | 0.015 |
| nsp7 | 3884 | | 0.01 |
| nsp9 | 4241 | | 0.016 |

TABLE 9A
TCE10 Cassette (Order of Frames as Shown)

| | Frame Start | Frame End | | SEQ |
|---|---|---|---|---|
| Gene | in Gene | in Gene | Frame sequence | ID NO: |
| N | 356 | 378 | HIDAYKTFPPTEPKKDKKKKADE | 27944 |
| ORF3a | 133 | 149 | CRSKNPLLYDANYFLCW | 27945 |
| nsp3 | 1632 | 1660 | FEYYHTTDPSFLGRYMSALNHTKKWKYPQ | 27946 |
| nsp3 | 1360 | 1377 | ISNEKQEILGTVSWNLRE | 27947 |
| M | 160 | 203 | DIKDLPKEITVATSRTLSYYKLGASQRVAGDSGFA | 27948 |
| | | | AYSRYRIGN | |
| ORF3a | 29 | 52 | VRATATIPIQASLPFGWLIVGVAL | 27949 |
| nsp4 | 3111 | 3134 | YLTFYLTNDVSFLAHIQWMVMFTP | 27950 |
| M | 89 | 109 | GLMWLSYFIASFRLFARTRSM | 27951 |
| nsp12 | 4553 | 4571 | DWYDFVENPDILRVYANLG | 27952 |
| nsp3 | 1919 | 1935 | PYPNASFDNFKFVCDNI | 27953 |
| nsp12 | 4806 | 4826 | NFNKDFYDFAVSKGFFKEGSS | 27954 |
| nsp12 | 4728 | 4745 | DGVPFVVSTGYHFRELGV | 27955 |
| nsp4 | 2850 | 2876 | ACPLIAAVITREVGFVVPGLPGTILRT | 27956 |
| nsp3 | 2745 | 2761 | ATTRQVVNVVTTKIALK | 27957 |
| N | 318 | 337 | SRIGMEVTPSGTWLTYTGAI | 27958 |
| M | 61 | 77 | TLACFVLAAVYRINWIT | 27959 |
| N | 96 | 117 | GGDGKMKDLSPRWYFYYLGTGP | 27960 |

TABLE 9B
TCE9 Cassette (Order of Frames as Shown)

| | Frame Start | Frame End | | SEQ |
|---|---|---|---|---|
| Gene | in Gene | in Gene | Frame sequence | ID NO: |
| nsp12 | 4719 | 4745 | GPLVRKIFVDGVPFVVSTGYHFRELGV | 27961 |
| nsp4 | 3096 | 3134 | PVYSFLPGVYSVIYLYLTFYLTNDVSFLAHIQWMV | 27962 |
| | | | MFTP | |
| M | 89 | 109 | GLMWLSYFIASFRLFARTRSM | 27951 |
| nsp12 | 4888 | 4905 | NNLDKSAGFPFNKWGKAR | 27963 |
| nsp3 | 2745 | 2761 | ATTRQVVNVVTTKIALK | 27957 |
| M | 61 | 79 | TLACFVLAAVYRINWITGG | 27964 |
| nsp3 | 1919 | 1935 | PYPNASFDNFKFVCDNI | 27953 |
| nsp3 | 2676 | 2692 | TYNKVENMTPRDLGACI | 27965 |
| ORF3a | 29 | 52 | VRATATIPIQASLPFGWLIVGVAL | 27949 |
| nsp12 | 4806 | 4826 | NFNKDFYDFAVSKGFFKEGSS | 27954 |
| N | 96 | 117 | GGDGKMKDLSPRWYFYYLGTGP | 27960 |
| nsp12 | 5213 | 5234 | KQGDDYVYLPYPDPSRILGAGC | 27966 |
| N | 356 | 378 | HIDAYKTFPPTEPKKDKKKKADE | 27944 |
| ORF3a | 133 | 149 | CRSKNPLLYDANYFLCW | 27945 |
| nsp6 | 3643 | 3668 | SLATVAYFNMVYMPASWVMRIMTWLD | 27967 |
| nsp12 | 4529 | 4545 | GNCDTLKEILVTYNCCD | 27968 |
| nsp3 | 1630 | 1660 | EAFEYYHTTDPSFLGRYMSALNHTKKWKYPQ | 27969 |
| nsp3 | 1360 | 1377 | ISNEKQEILGTVSWNLRE | 27947 |
| M | 160 | 203 | DIKDLPKEITVATSRTLSYYKLGASQRVAGDSGFA | 27948 |
| | | | AYSRYRIGN | |
| N | 301 | 337 | WPQIAQFAPSASAFFGMSRIGMEVTPSGTWLTYTG | 27970 |
| | | | AI | |
| nsp12 | 4553 | 4571 | DWYDFVENPDILRVYANLG | 27952 |
| nsp4 | 2850 | 2909 | ACPLIAAVITREVGFVVPGLPGTILRTTNGDFLHELP | 27971 |
| | | | RVFSAVGNICYTPSKLIEYTDFA | |

TABLE 9C
TCE11 Cassette (Order of Frames as Shown)

| | Frame Start | Frame End | | |
|---|---|---|---|---|
| Gene | in Gene | in Gene | Frame sequence | SEQ ID NO: |
| nsp12 | 4896 | 4826 | NVAFQTVKPGNFNKDFYDFAVSKGFFKEGSS | 27972 |
| M | 182 | 203 | GASQRVAGDSGFAAYSRYRIGN | 27973 |
| nsp12 | 4728 | 4745 | DGVPFVVSTGYHFRELGV | 27955 |
| nsp4 | 3111 | 3134 | YLTFYLTNDVSFLAHIQWMVMFTP | 27950 |
| M | 89 | 109 | GLMWLSYFIASFRLFARTRSM | 27951 |
| nsp3 | 1632 | 1660 | FEYYHTTDPSFLGRYMSALNHTKKWKYPQ | 27946 |
| nsp3 | 1360 | 1377 | ISNEKQEILGTVSWNLRE | 27947 |
| nsp3 | 2745 | 2761 | ATTRQVVNVVTTKIALK | 27957 |
| M | 61 | 77 | TLACFVLAAVYRINWIT | 27959 |

TABLE 10A
Population Coverages for SARS-CoV-2 Validated
Epitopes (Excluding mutations >5%)

| Cassette | Magnitude | Starting Protein(s) | AFA | API | EUR | HIS | Mean | Min |
|---|---|---|---|---|---|---|---|---|
| Spike | 1 | S | 0.95 | 0.89 | 1 | 0.98 | 0.95 | 0.89 |
| S + N + TCE11 | 1 | S, N | 0.97 | 0.95 | 1 | 0.99 | 0.98 | 0.95 |
| S + TCE10 | 1 | S | 0.97 | 0.95 | 1 | 0.99 | 0.98 | 0.95 |
| S + TCE9 | 1 | S | 0.97 | 0.95 | 1 | 0.99 | 0.98 | 0.95 |
| S + TCE5 | 1 | S | 0.95 | 0.95 | 1 | 0.98 | 0.97 | 0.95 |
| Spike | 100 | S | 0.92 | 0.86 | 1 | 0.96 | 0.93 | 0.86 |
| S + N + TCE11 | 100 | S, N | 0.95 | 0.95 | 1 | 0.99 | 0.97 | 0.95 |
| S + TCE10 | 100 | S | 0.95 | 0.95 | 1 | 0.99 | 0.97 | 0.95 |
| S + TCE9 | 100 | S | 0.95 | 0.95 | 1 | 0.99 | 0.97 | 0.95 |
| S + TCE5 | 100 | S | 0.92 | 0.94 | 1 | 0.97 | 0.96 | 0.92 |
| Spike | 1000 | S | 0.6 | 0.62 | 0.91 | 0.75 | 0.72 | 0.6 |
| S + N + TCE11 | 1000 | S, N | 0.89 | 0.92 | 1 | 0.96 | 0.94 | 0.89 |
| S + TCE10 | 1000 | S | 0.9 | 0.92 | 1 | 0.97 | 0.95 | 0.9 |
| S + TCE9 | 1000 | S | 0.9 | 0.92 | 1 | 0.97 | 0.95 | 0.9 |
| S + TCE5 | 1000 | S | 0.68 | 0.7 | 0.93 | 0.8 | 0.78 | 0.68 |
| Spike | 2000 | S | 0.39 | 0.49 | 0.73 | 0.56 | 0.54 | 0.39 |
| S + N + TCE11 | 2000 | S, N | 0.66 | 0.72 | 0.96 | 0.82 | 0.79 | 0.66 |
| S + TCE10 | 2000 | S | 0.72 | 0.73 | 0.96 | 0.85 | 0.81 | 0.72 |
| S + TCE9 | 2000 | S | 0.76 | 0.77 | 0.98 | 0.9 | 0.85 | 0.76 |
| S + TCE5 | 2000 | S | 0.49 | 0.54 | 0.81 | 0.66 | 0.62 | 0.49 |

TABLE 10B
Population Coverages for Validated Conserved Epitopes
between SARS and SARS-CoV-2 (Excluding mutations >5%)

| Cassette | Magnitude | Starting Protein(s) | AFA | API | EUR | HIS | Mean | Min |
|---|---|---|---|---|---|---|---|---|
| Spike | 1 | S | 0.74 | 0.82 | 0.98 | 0.91 | 0.87 | 0.74 |
| S + N + TCE11 | 1 | S, N | 0.89 | 0.93 | 1 | 0.97 | 0.95 | 0.89 |
| S + TCE10 | 1 | S | 0.94 | 0.93 | 1 | 0.97 | 0.96 | 0.93 |
| S + TCE9 | 1 | S | 0.94 | 0.93 | 0.99 | 0.97 | 0.96 | 0.93 |
| S + TCE5 | 1 | S | 0.87 | 0.91 | 0.99 | 0.95 | 0.93 | 0.87 |
| Spike | 100 | S | 0.74 | 0.78 | 0.98 | 0.89 | 0.85 | 0.74 |
| S + N + TCE11 | 100 | S, N | 0.89 | 0.91 | 0.99 | 0.95 | 0.94 | 0.89 |
| S + TCE10 | 100 | S | 0.94 | 0.93 | 0.99 | 0.97 | 0.96 | 0.93 |
| S + TCE9 | 100 | S | 0.94 | 0.92 | 0.99 | 0.97 | 0.96 | 0.92 |
| S + TCE5 | 100 | S | 0.86 | 0.88 | 0.98 | 0.93 | 0.91 | 0.86 |
| Spike | 1000 | S | 0.18 | 0.2 | 0.46 | 0.27 | 0.28 | 0.18 |
| S + N + TCE11 | 1000 | S, N | 0.49 | 0.67 | 0.76 | 0.62 | 0.63 | 0.49 |
| S + TCE10 | 1000 | S | 0.52 | 0.56 | 0.71 | 0.55 | 0.59 | 0.52 |
| S + TCE9 | 1000 | S | 0.74 | 0.81 | 0.92 | 0.84 | 0.83 | 0.74 |
| S + TCE5 | 1000 | S | 0.33 | 0.42 | 0.62 | 0.49 | 0.47 | 0.33 |
| Spike | 2000 | S | 0.01 | 0.03 | 0.08 | 0.03 | 0.04 | 0.01 |
| S + N + TCE11 | 2000 | S, N | 0.24 | 0.2 | 0.41 | 0.28 | 0.28 | 0.2 |
| S + TCE10 | 2000 | S | 0.2 | 0.15 | 0.34 | 0.17 | 0.21 | 0.15 |
| S + TCE9 | 2000 | S | 0.4 | 0.37 | 0.7 | 0.51 | 0.49 | 0.37 |
| S + TCE5 | 2000 | S | 0.21 | 0.11 | 0.34 | 0.24 | 0.22 | 0.11 |

XIV.I. SARS-COV-2 Prime-Boost Regimens Featuring Spike Proteins from Different SARS-COV-2 Subtype Isolates Produces Responses to Spike in Non-Human Primates

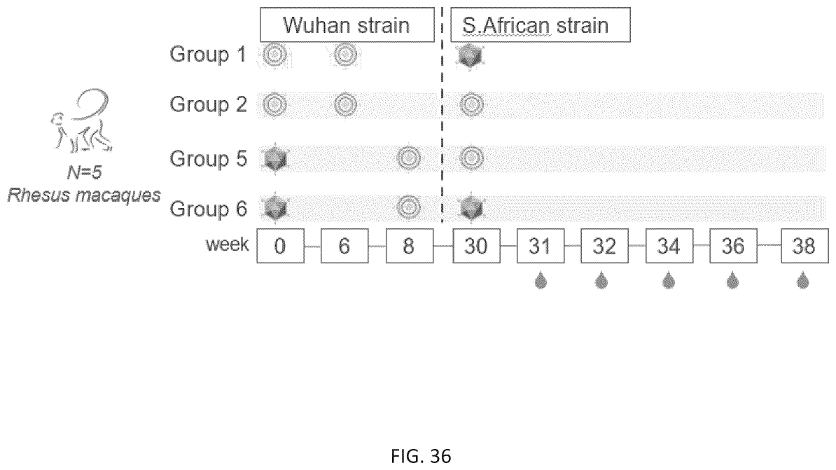

ChAd and SAM vaccine platforms encoding different subtype isolates of the SARS-CoV-2 Spike protein were assessed in Indian rhesus macaques as part of homologous or heterologous prime/boost regimens, as shown in FIG. 36 and presented in Table 11.

TABLE 11
NHP Study Design for SARS-CoV-2 Isolate Vaccine Regimens

| Group # | Prime (week 0) | Boost 1 (week 6 or 8) | Boost 2 (week 30) |
|---|---|---|---|
| 1 | SAM-S_{D614G}; 300 ug | SAM-S_{D614G}; 300 ug | ChAd-S_{B1351}-TCE5; 5e11 vp |
| 2 | SAM-S_{D614G}; 30 ug | SAM-S_{D614G}; 30 ug | SAM-TCE5-S_{B1351}; 30 ug |
| 5 | ChAd-S_{D614G}; 1e12 vp | SAM-S_{D614G}; 100 ug | SAM-TCE5-S_{B1351}; 30 ug |
| 6 | ChAd.S_{D614G}; 5e10 vp | SAM-S_{D614G}; 100 ug | ChAd-S_{B1351}-TCE5; 5e11 vp |

Prime: ChAdV - “CTSpike_{g}” (SEQ ID NO: 79); SAM - “IDTSpike_{g}” (SEQ ID NO: 69)
Boost 1: SAM - “IDTSpike_{g}” (SEQ ID NO: 69)
Boost 2: all CT-F2P versions of Spike variant B.1.351; TCE5 see Table 7

NHPs were first immunized with a priming dose of either a ChAd platform including a Spike-encoding cassette featuring “ChAd-S_{D614G}; CT” (SEQ ID NO:79) or a SAM platform including a Spike-encoding cassette featuring “SAM-S_{D614G}; IDT” (SEQ ID NO:69) at the indicated doses. NHPs were then administered a first boost at weeks 6 or 8 with the SAM platform including a Spike-encoding cassette featuring “SAM-S_{D614G}; IDT” at the indicated doses. NHPs were then administered a second boost at week 30 with either a ChAd platform including a B.1.351 Spike variant-encoding cassette featuring Cool Tool sequence optimization (“CT”) and the F2P modification described herein (“F2P”) [SEQ ID NO:112] or a SAM platform including the same B.1.351 Spike variant (each platform also included the TCE5 T cell epitope cassette, see Table 7, in the orientation shown). The ChAdV antigen cassette is shown in SEQ ID NO:113. NHPs were monitored over time, as described herein.

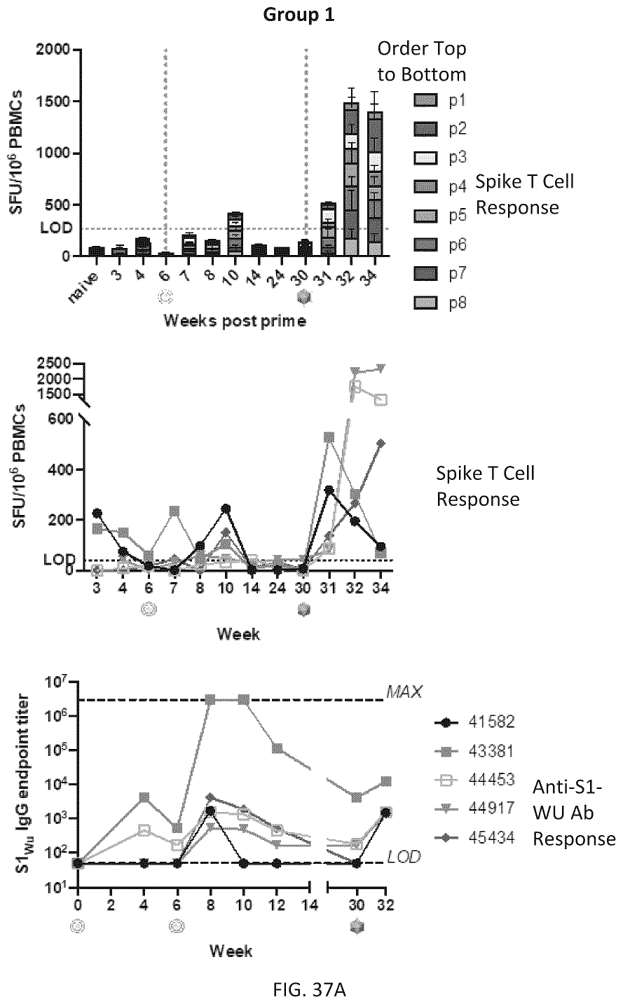

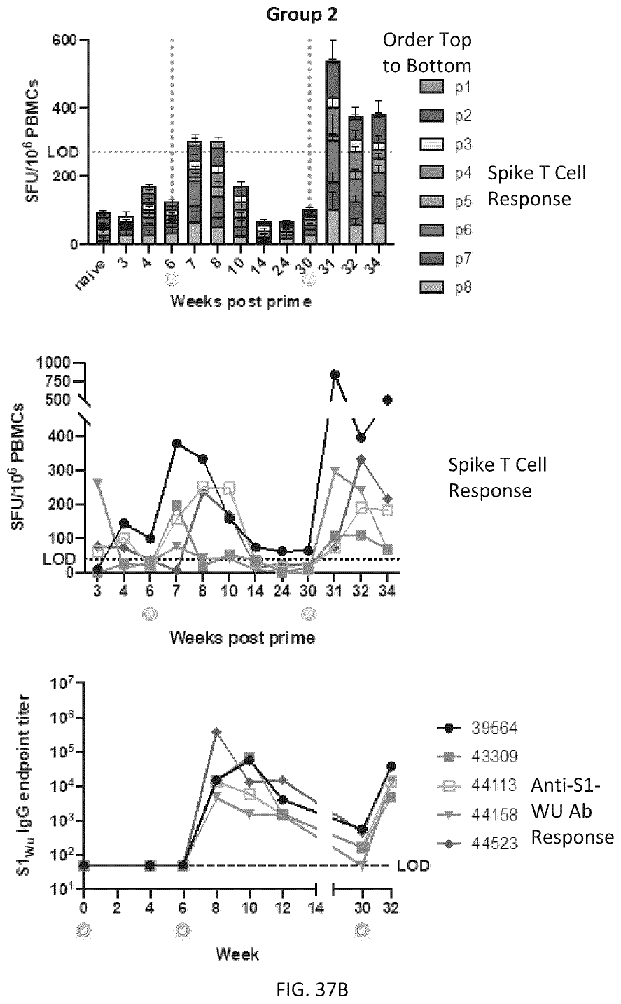

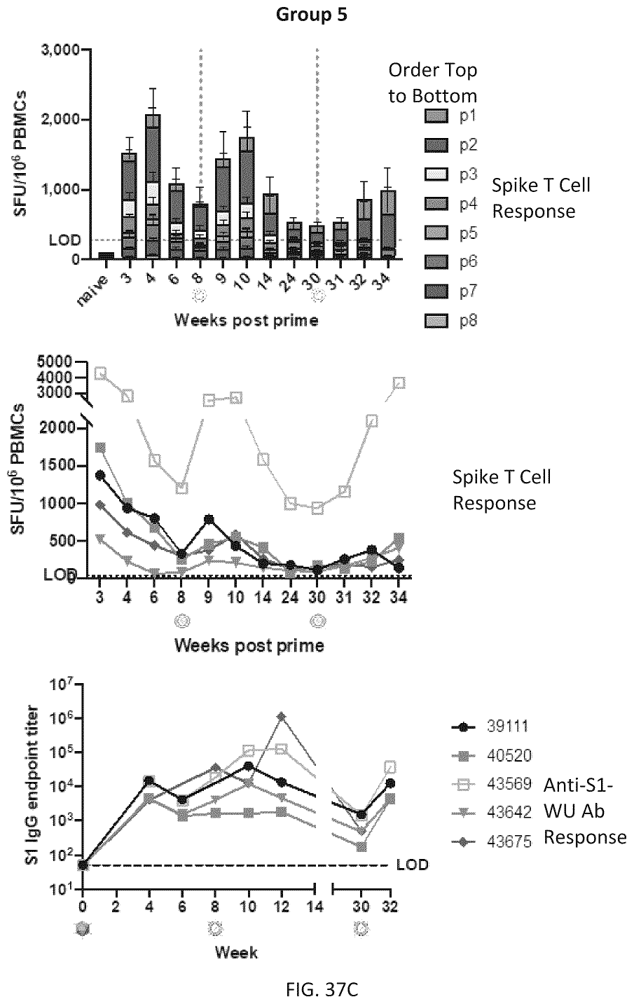

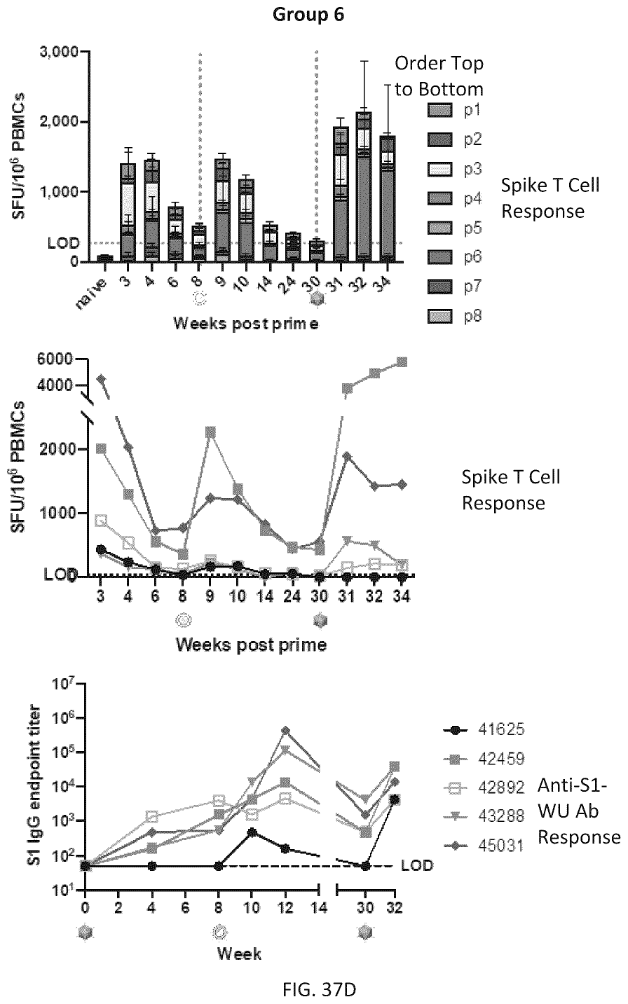

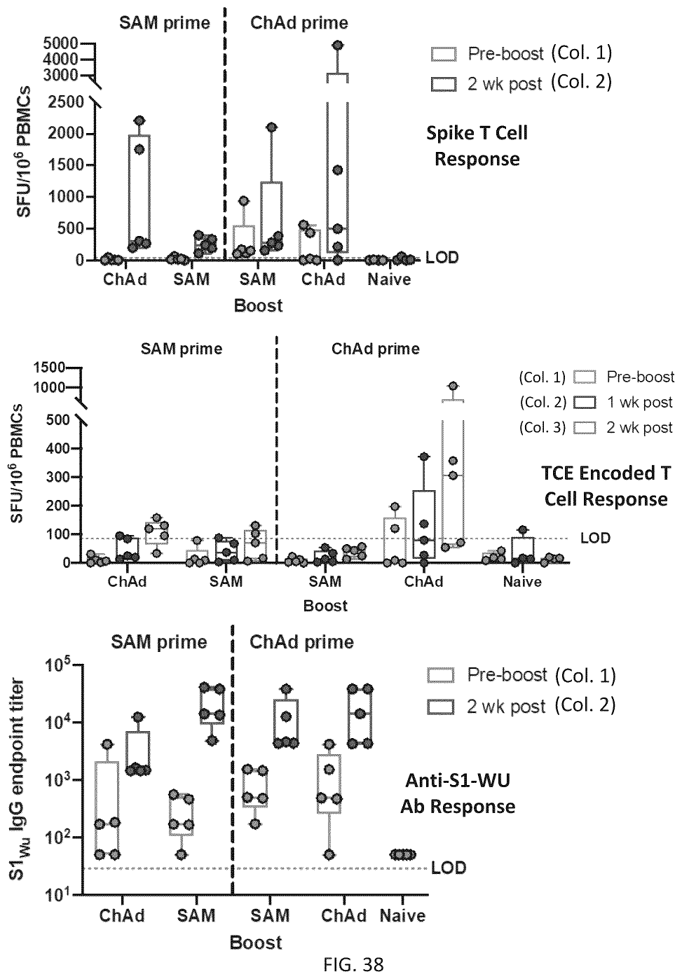

As shown in FIGS. 37A, 37B, 37C, and 37D, the various vaccine regimens (Groups 1, 2, 5, and 6, respectively) produced T cell responses across multiple Spike T cell epitope pools (top panels). T cell responses for individual NHPs directed to a single large Spike T cell epitope pool was heterogenous (middle panels and summarized in FIG. 38 top panel), with each boost generally producing an increased T cell response, including production of a robust response in some (e.g., two NHPs in Group 1 following Boost 2). Spike-specific IgG antibody titers were detected and increased following each boost (bottom panels and summarized in FIG. 38 bottom panel) in all five NHP animals assessed. T cell responses to the TCE5-encoded epitopes, though generally small, trended upwards following Boost 2 (the first administration of a vaccine including TCE5), with generally stronger responses with administration of the ChAdV platform vaccine (FIG. 38 middle panel). Accordingly, the data demonstrate a vaccine regimen including a boost with a Spike variant encoding vaccine produced T cell and antibody responses.

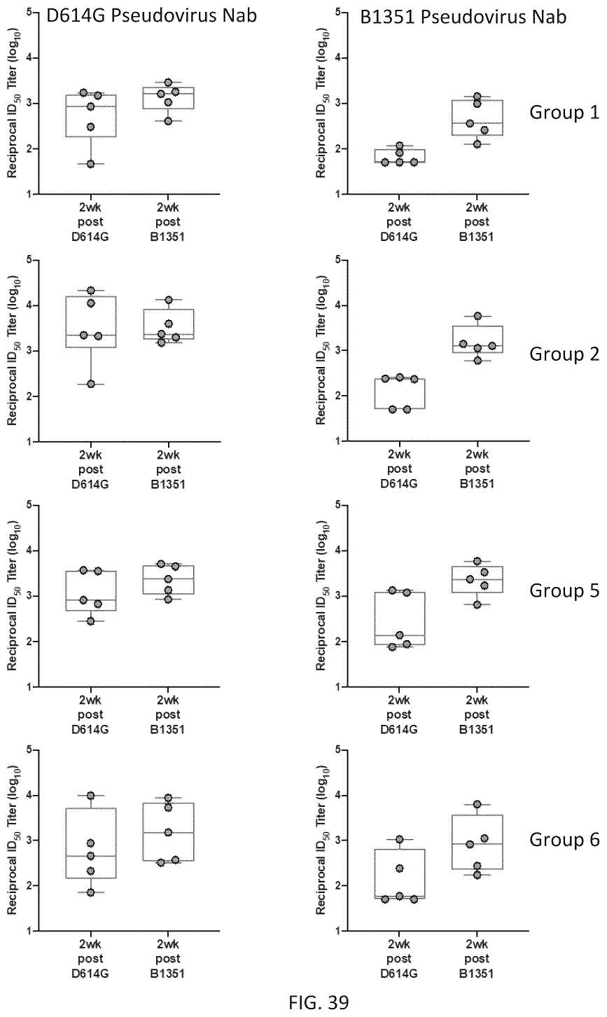

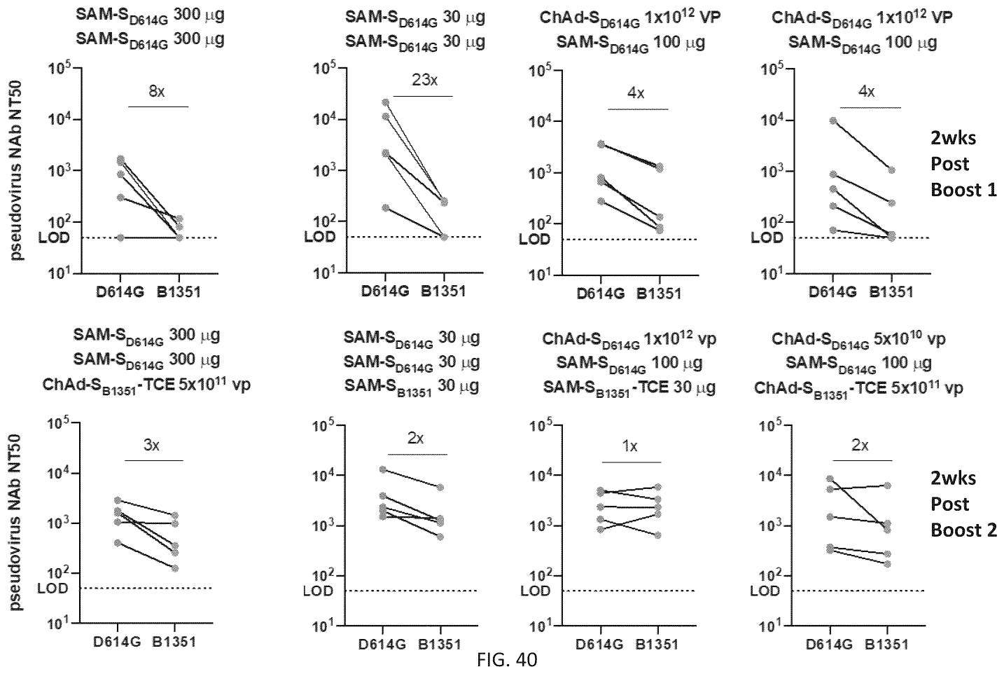

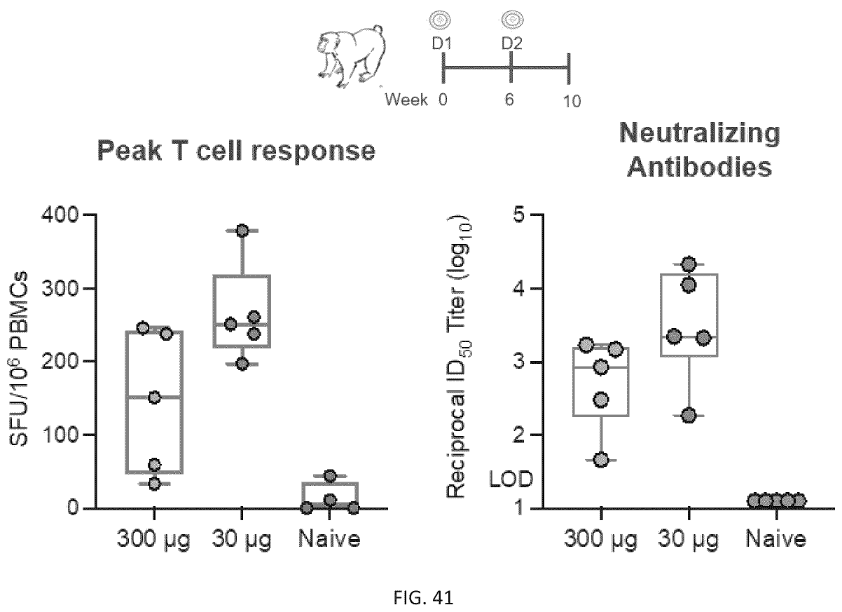

Antibody responses were further assessed for neutralizing antibody production to both the D614G pseudovirus and B.1.351 pseudovirus. As shown in FIG. 39, neutralizing antibody (Nab) titers against the D614G pseudovirus were detected following Boost 1 across the four groups, with Nab titers generally the same following Boost 2 (left panels). Following Boost 1, cross-neutralizing antibody titers against the B.1.351 pseudovirus, while detected, were distinctly lower than the Nab titer against the D614G pseudovirus (right panel, column 1). However, following administration of Boost 2 encoding the B.1.351 spike variant, Nab titers against the B.1.351 pseudovirus were noticeably increased (right panels, column 2), notably with similar Nab titer levels to that against the D614G pseudovirus following Boost 1. These results are further demonstrated in FIG. 40, comparing the relative Nab titer levels against each of the pseudoviruses illustrating the reduced cross-neutralizing capacity against the B. 1.351 pseudovirus following Boost 1 (top panels) and rescuing of the same following Boost 2 (bottom panels) across each of the vaccine regimens assessed. FIG. 41 shows T cell responses (left panel) and Nab titer levels (right panel) for Rhesus macaques immunized twice with SAM encoding SARS-COV-2 Spike antigen at a specified dose of either 30 μg or 300 μg, with 30 μg doses producing a more robust response.

The data demonstrate the various vaccine regimens produced both T cell and antibody responses against the encoded antigens in NHPs, notably demonstrating subsequent immunization with Spike variant encoding vaccines noticeably improved Nab titers against the respective variant pseudovirus.

### XIV.J. Multivalent Pancorona Immunogen (Full Length Spike, RBDs & TCEs) Design Based on Sequence Analysis & T-Cell Epitope Prediction Overview

Vaccine candidates were developed to provide broad immunogenicity against viruses in the Sarbecovirus subgenus, which includes the human pathogens SARS-COV and SARS-CoV-2. Vaccine candidates were constructed to induce both broadly neutralizing antibodies and CD8+ T-cell responses to increase protection against diverse Sarbecoviruses. For that purpose, two classes of vaccine cassettes were developed: the first to elicit cross-neutralizing anti-Spike and anti-RBD antibodies capable of preventing infection, and the second to generate broad CD8+ T-cell responses to conserved T-cell epitopes, capable of quickly destroying infected cells thereby limiting infection severity.

A summary of the relevant findings from the results below are as follows:
Sarbecovirus-RBDs were expressed from SAM vectors with different Trimerization domains and assembled into Trimers in vitro, which mirrors the likely natural trimerization conformation of full length spike proteins, presenting the immunogens in a desired format for immunogenicity purposes
Vaccination with single RBD and blended multi-RBDs SAM vaccines generated antibody and neutralizing immunity to targeted RBD proteins, and also to proteins not directly targeted by the vaccines
The 3 vaccine groups results suggest induction of comparable immunity
Neutralizing immunity significantly increased by homologous boosting of blended SAMs, even to target bat species not originally included in the vaccine
Pre-existing SARS-COV-2 or SARS-1 immunity led to greater breath of immune response with a single vaccination with the pooled SAM-vectors
A single SAM vector expressing a SARS-COV2 spike and 3×RBDs from groups 1, 2 and 3 induced immunity to multiple RBDS and neutralizing titers beyond those targeted by the vaccine
A single ChAd vector expressing a SARS-COV2 spike and 3×RBDs from group 2 induced broad neutralizing immunity to multiple viruses that is improved by SAM boosting
TCE demonstrated expression by RT-QPCR with TCE12 having the best expression of the TCEs evaluated
TCE12 and TCE13 induced potent T-cell immunity in mice to multiple conserved Nsp proteins and SAM-TCE12 induced a comparable immune response to ChAd-TCE12

### XIV.K. Multivalent Pancorona Immunogen Assessment Methods

### Cloning of Single RBD Constructs

RBDs were cloned by Gibson assembly into SAM and ChAd vaccine vectors for in vitro and in vivo testing, and into pCDNA3 expression vectors to generate reagents for immunodetection. RBD domains from Arg319 to Phe541 (Wuhan-SARS-COV2 numbering or equivalent amino acids) were selected for cloning which contains the core RBD domain between 333-527 amino acids (Wuhan SARS-COV-2 numbering) (Lan et al. 220 Nature 581:215-22).

RBD sequences were codon optimized using the COOL algorithm and were synthesized as gBlocks by IDT. Trimerization domains were introduced initially on the gBlocks but additional trimerization domains for each RBD were introduced by overlapping PCR extension. RBDs were then assembled into PacI/BstBI linearized pUC02-ATG-VEE linearized backbones by Gibson assembly. A subset of RBDs were moved from SAM to ChAd using primers to introduce overlapping regions needed for constructs assembly into AsiSI/PmeI restriction sites.

For pCNA3 cloning the RBD was first amplified by PCR to introduce overlapping domains for assembly and to introduce a C-terminus 6-Histidine residue domain for protein purification and then cloned into the BsRGI/HindIII site. Protein was expressed from the pCDNA3 plasmids using a 293Expi expression system and RBDs were purified using a Nickel column by FPLC.

### In Vitro Expression of RBDs and Westerns

One μg of SAM RNA was transfected for each construct under evaluation. HEK293A cells were seeded at 2.5e5 cells/well of a 24 well plate and incubated overnight. The next day, 1 μg of RNA was transfected using Lipofectamine Messenger Max (Thermofisher) according to the manufacturer's instructions. Media and pellets were harvested and Laemmli buffer was added to 1X. For the reduced samples Laemmli, 1 mM DTT and 4M Urea were added to the media. Reduced samples were also heated to 95 C for 5 minutes while the non-reduced samples were analyzed unheated. Samples were separated on a 4-20% SDS-PAGE gel and transferred to a PVDF membrane. Membranes were blocked in 5% Skim milk TBST and then probed with a rabbit anti-RBD polyclonal antibody (SinoBiologicals) or an anti-His Tag monoclonal antibody (Invitrogen). Antibody binding was detected with either a HRP-labeled anti-rabbit or anti-mouse secondary antibody (Bethyl Labs). Signal was detected using a Chemiluminescent substrate (Thermoscientific).

### In Vitro Evaluation of TCE Expression by RT-qPCR

For each construct, 1e6 293F cells were infected with the corresponding ChAdV-TCE virus at an MOI of 1. After 48 hours, the cells were harvested for RNA extraction and purification. The purified RNA from each construct was used to generate cDNA via reverse transcription. 1 ng of cDNA for each construct was used as a template for two separate qPCR reactions (done in triplicate); a FAM coupled primer set targeting the PADRE sequence and a FAM coupled primer set targeting b-Actin as a normalizing control.

To determine the expression of PADRE for each construct relative to the expression of actin, the CT value of the b-Actin reaction was subtracted from the average value of the PADRE reaction to provide the deltaCT (dCT) value for each construct. The dCT value for the untransduced control was subtracted from the dCT values for each construct to provide the double deltaCT (ddCT) value for each construct. To determine the relative level of PADRE expression above the baseline seen in the untransduced control, the ddCT values were converted to expression levels above baseline via the following formula: Relative PADRE expression level=2^{-(ddCT)}. The relative PADRE expression levels shown are shown as a proportion of the highest relative PADRE expression level, seen in the GRT-C903 positive control.

### Pseudovirus Neutralization Assay (PNA)

Mouse sera samples were assessed using a pseudovirus assay. Five sarbecovirus (Human SARS-COV2, Human SARS-COV1, bat MK211376, bat WIV-1, bat RSSHC014, bat Rs/TG13, and Pangolin GX-P2V) pseudotyped virus particles were made using a genetically modified Vesicular Stomatitis Virus from which the glycoprotein G was removed (VSVAG). The VSVAG virus was transduced in HEK293T cells previously transfected with the spike glycoprotein of the SARS-COV-2 coronavirus (Wuhan strain) for which the last 19 amino acids of the cytoplasmic tail were removed (ΔCT). The generated pseudovirus particles (VSVΔG-Spike ΔCT) contained a luciferase reporter which could be quantified in relative luminescence units (RLU). Heat-inactivated serum samples were serially diluted (7-serial 2-fold dilution) in a 96-well plate and a pre-determined amount of pseudotyped virus (corresponding to approximately 150,000 RLU/well) was applied to the plate and incubated with scrum/plasma to allow binding of the neutralization antibodies to the pseudotyped virus.

After the incubation of the serum/plasma-pseudotyped virus complex, the scrum/plasma-pseudotyped virus complex was transferred to the plate containing Vero E6 cells (ATCC). Test plates were incubated at 37° C. with 5% CO2 overnight. Luciferase substrate was added to the plates which were then read using a plate reader detecting luminescence. The intensity of the light being emitted is inversely proportional to the amount of anti-SARS-COV-2 Pre-Spike antibodies bound to the VSVΔG-Spike ΔCT particles.

Each microplate was read using a luminescence microplate reader (SpectraMax). The dilution of serum required to achieve 50% neutralization (NT50) when compared to a non-neutralized pseudoparticle control was calculated for each sample dilution and the NT50 is interpolated from a linear regression using the two dilutions flanking the 50% neutralization.

### Anti-RBD MSD ELISA

96-well QuickPlex plates (Meso Scale Discovery, Rockville, MD) were coated with 50 μL of 1 μg/mL SARS-COV-2 S1 (ACROBiosystems, Newark, DE) or 15 Sarbecovirus RBDs (produced by expression of 6-His tagged RBD proteins using a pCDNA3 plasmid in 293Expi expression system), diluted in DPBS (Corning, Corning, NY), and incubated at 4° C. overnight. Wells were washed three times with agitation using 250 μL of PBS+0.05% Tween-20 (Teknova, Hollister, CA) and plates blocked with 150 μL Superblock PBS (Thermo Fisher Scientific, Waltham, MA) for 1 hour at room temperature on an orbital shaker. Test sera was diluted at appropriate series in 10% species-matched serum (Innovative Research, Novi, MI) and tested in single wells on each plate. Wells were washed and 50 μL of the diluted samples were added to wells and incubated for 1 hour at room temperature on an orbital shaker. Wells were washed and incubated with 25 μL of 1 μg/mL SULFO-TAG labeled anti-mouse antibody (MSD), diluted in DPBS+1% BSA (Sigma-Aldrich, St. Louis, MO), for 1 hour at room temperature on an orbital shaker. Wells were washed and 150 μL tripropylamine containing read buffer (MSD) added. Plates were run immediately using the QPlex SQ 120 (MSD) ECL plate reader.

### TCE ELISpot

IFNγ ELISpot assays were performed using pre-coated 96-well plates (MAbtech, Mouse IFNγ ELISpot PLUS, ALP) following manufacturer's protocol. Samples were stimulated overnight with different overlapping 15mer peptide pools spanning the entirety of various NSP epitope frames used in the vaccine cassettes (8, 12, 13, 14) and NC, 15 amino acid peptides per pool, with 11 amino acid overlap (JPT Peptide Technologies and GenScript). All peptide pools were used at a final concentration of 1 mg/mL per peptide. Splenocytes were plated at 1×10⁵ cells per well for TCE pools in the initial ChAd experiment with the exception that in the SAM versus ChAd-TCE12 experiment NSP12 pulsed cells were plated in duplicate at 2.5×10⁴ cells per well (mixed with 7.5×10⁴ naïve cells). DMSO only control was plated for each sample and cell number. Following overnight incubation at 37° C., plates were washed with PBS and incubated with anti-mouse IFNγ mAb biotin (MAbtech) for two hours, followed by an additional wash and incubation with Streptavidin-ALP (MAbtech) for one hour. After final wash, plates were incubated for ten minutes with BCIP/NBT (MAbtech) to develop the immunospots. Spots were imaged and enumerated using an AID reader (Autoimmun Diagnostika). For data processing and analysis, samples with replicate well variability (Variability=Variance/(median+1)) greater than 10 and median greater than 10 were excluded. Spot values were adjusted based on the well saturation according to the formula: AdjustedSpots=RawSpots+2*(RawSpots*Saturation/(100−Saturation). Wells with well saturation values greater than 39% were labeled as “too numerous to count” (TNTC) and set to the maximum measured value (8,000 SFU/10⁶ for samples plated at 1×10⁵ cells/well and 27,000 SFU/10⁶ for samples plated at 25,000 cells/well). Each sample was background corrected by subtracting the average value of the negative control peptide wells. Data processing performed with R programming language and graphed with GraphPad Prism 9. Data is presented as spot forming units (SFU) per 1×10⁶ splenocytes.

### XIV.L. SARS-COV-2 Spike and Multi-RBD Cassette Design

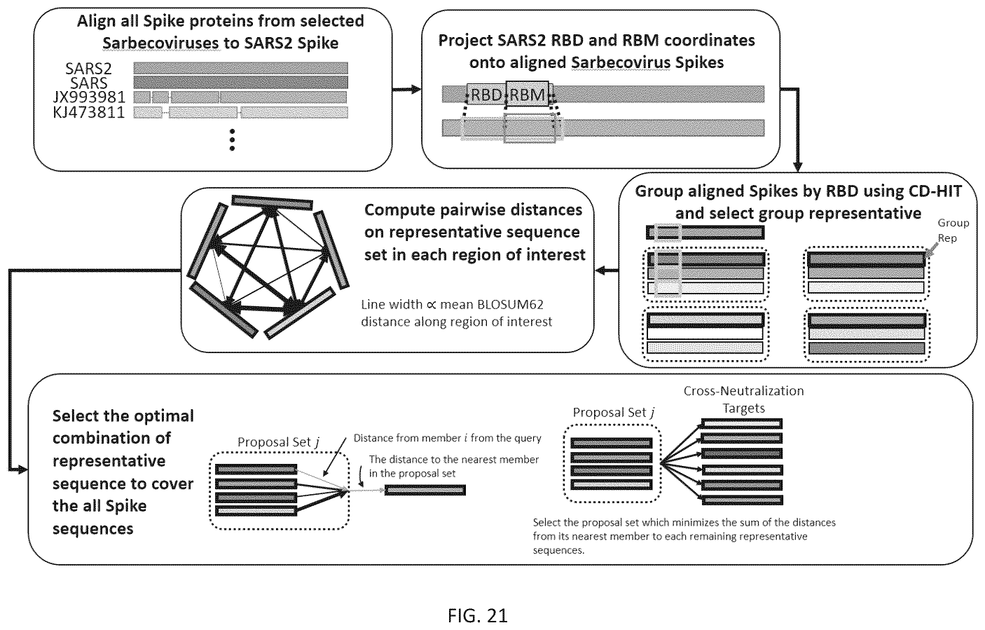

The design for these target RBDs was based on multiple alignment analysis of 75 high quality Sarbecovirus Spike sequences from NCBI. The genetic distances between any two viruses were inferred from the alignment. As outlined in FIG. 21, a statistical model selected three RBD sequences from the collection with the objective that the three RBDs together with the SARS-COV-2 RBD sequence provide the most diverged group of sequences, such that the average distance between any virus to the most similar virus in the selected RBD group was smallest. This optimization strategy considers that any newly emerging Sarbecovirus is likely to be sufficiently similar to any of the RBD sequences in the cassette such that they can be neutralized by the breadth of the antibodies elicited by a vaccine encoding the selected RBDs. Different RBD domains were used for selection based on having different properties. The RBM is the most variable region of the RBD while the area outside of the RBM is more conserved. Without wishing to be bound by theory, selecting RBDs based on the RBM alone can result in selection based on the most varied domain, while choosing based on the RBD sequence outside of the RBM (RBDΔRBM) can result in a selection based on the conservative domain. Both selection criteria have potential advantages. More conserved regions may be better immunogens for antibodies that recognize multiple coronaviruses. Selection based on RBM may provide greater coverage to diverse RBMs. The whole RBD group can combine the advantages of both, but is potentially biased to the conserved domains.

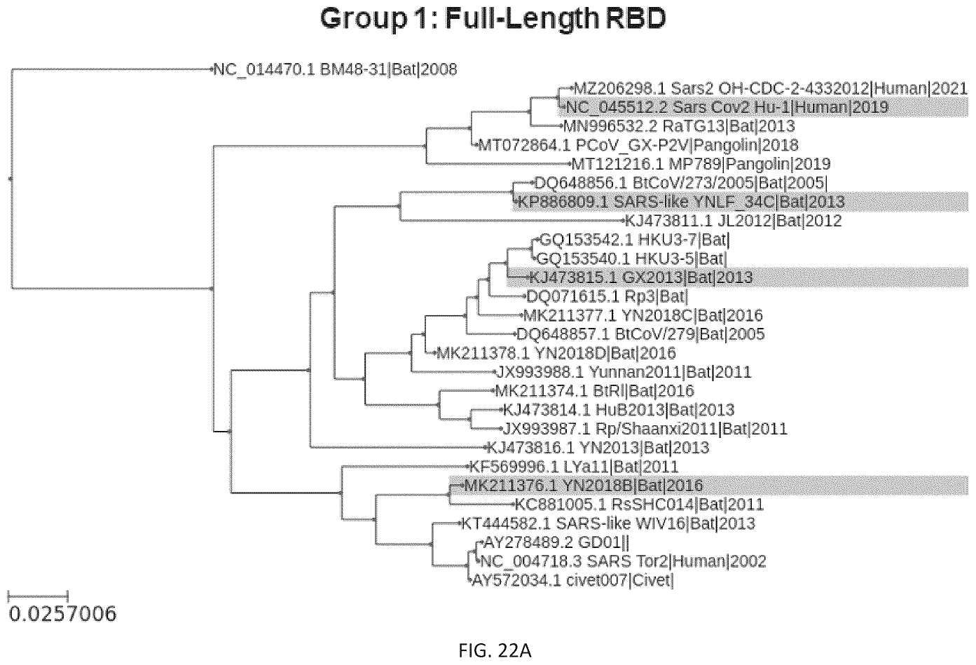

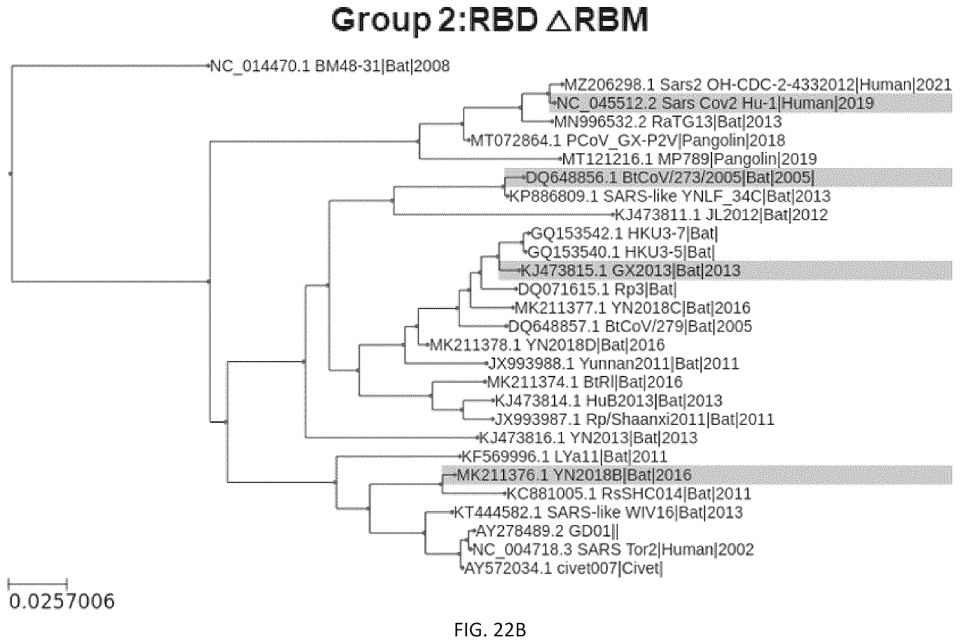

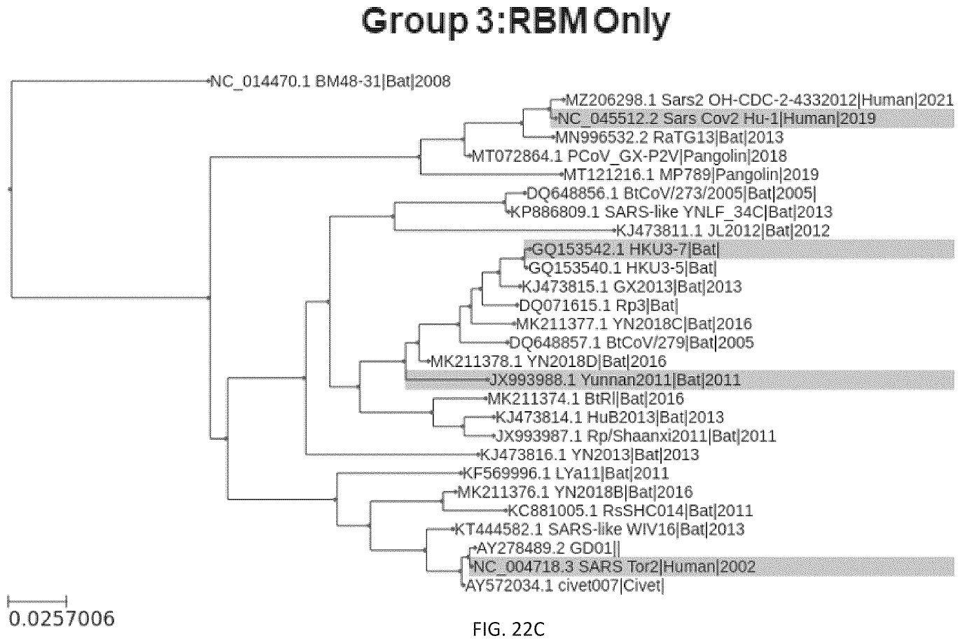

Using this distance model, three unique combinations of RBD candidates were selected based on the following sequences: 1) whole RBD sequence (FIG. 22A), 2) RBD sequence with the RBM sequence excluded (RBDΔRBM; FIG. 22B), and 3) RBM sequence alone (FIG. 22C). The selected RBDs are described in Table 12.

TABLE 12
Selected RBD Components

| Group 1: Whole RBD | Group 2: RBDΔRBM | Group 3: RBM Alone |
|---|---|---|
| SARS-like | BtCoV/273/2005/ | HKU3-7 (Bat) |
| YNLF_34C/Bat/2013 | BAT2005 | GenBank# GQ153542 |
| GenBank# KP886809 | GenBank# DQ648856 | |
| GX2013/BAT/2013 | GX2013/BAT/2013 | Yunnan2011/Bat/2011 |
| GenBank# KJ473815 | GenBank# KJ473815 | GenBank# JX993988.1 |
| YN2018B/Bat/2016 | YN2018B/Bat/2016 | SARS Tor2/Human/2002 |
| GenBank# MK211376 | GenBank# MK211376 | GenBank# NC_004718 |

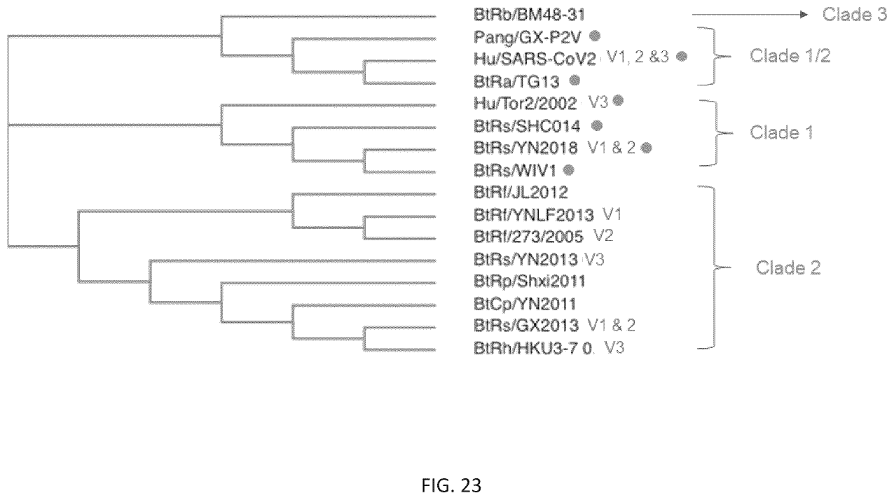

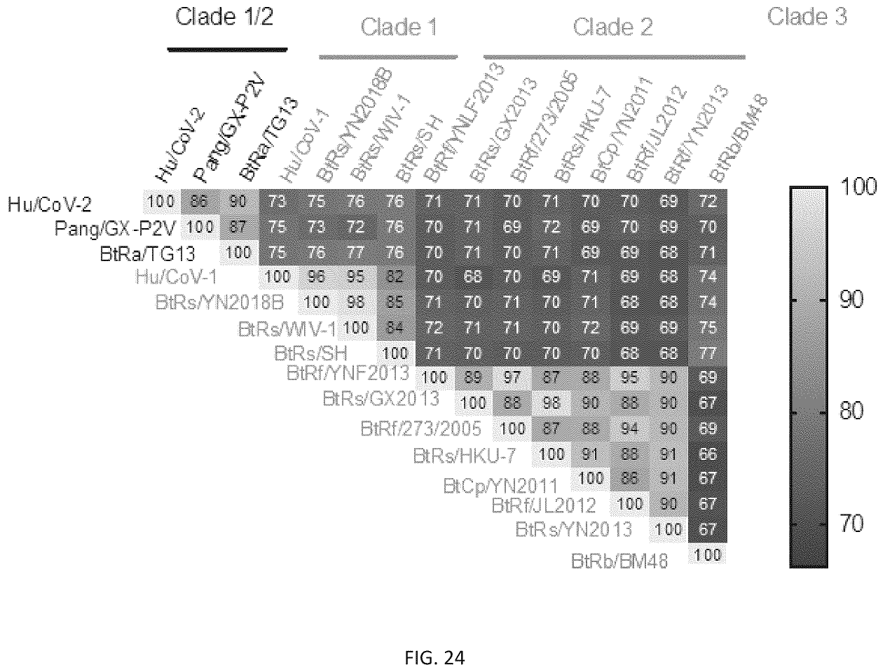

The sarbecovirus clade designations of representative analyzed isolates, including those with the selected RBDs, is shown in FIG. 23, with “V1”, “V2”, and “V3” referring to vaccine Groups 1-3, respectively, and circles indicating viruses for which PNA exists (see below). Clade designation was according to Letko et al. 2020 (Nature Microbiology volume 5, pages 562-569 (2020)). FIG. 24 shows RBD amino acid similarity for representative analyzed isolates, including those with the selected RBDs. Table 13 below presents the sequence identity of the best matching component (distinct RBD domain) of each vaccine set for particular viruses of interest. Tables 14A to 14C presents the pairwise sequence identity across the shown isolates for each of: the full-length RBD (Tables 14A); the RBDΔRBM (Tables 14B); the RBM alone (Tables 14C).

TABLE 13
Percent Identity to Viruses of Interest

| Group 1: Whole RBD | Group 2: RBDΔRBM | Group 3: RBM Alone |
|---|---|---|
| 74% identity to clade 3 | 74% identity to clade 3 | 74% identity to clade 3 |
| virus, BM48 | virus, BM48 | virus, BM48 |
| 98% identity to clade 1 | 98% identity to clade 1 | 95% identity to clade 1 |
| virus, WIV1 | virus, WIV1 | virus, WIV1 |
| 85% identity to clade 1 | 85% identity to clade 1 | 82% identity to clade 1 |
| virus, RsSH | virus, RsSH | virus, RsSH |
| 96% identity to clade 1 | 96% identity to clade 1 | 100% identity to clade 1 |
| virus SARS-1 | virus SARS-1 | virus SARS-1 |
| 90% identity to clade 2 | 90% identity to clade 2 | 91% identity to clade 2 |
| virus, YN2013 | virus, YN2013 | virus, YN2013 |

TABLE 14A
Percent Identity of RBD Domains

| | NC_045512.2 | NC_004718.3 | MT121216.1 | DQ648857.1 | GQ153542.1 | DQ648856.1 | AY278489.2 | GQ153540.1 |
|---|---|---|---|---|---|---|---|---|
| NC_045512.2 | 1 | 0.730942 | 0.96861 | 0.64574 | 0.650224 | 0.636771 | 0.730942 | 0.650224 |
| NC_004718.3 | 0.730942 | 1 | 0.73991 | 0.636771 | 0.636771 | 0.641256 | 0.986547 | 0.636771 |
| MT121216.1 | 0.96861 | 0.73991 | 1 | 0.641256 | 0.64574 | 0.632287 | 0.73991 | 0.64574 |
| DQ648857.1 | 0.64574 | 0.636771 | 0.641256 | 1 | 0.928251 | 0.874439 | 0.627803 | 0.928251 |
| GQ153542.1 | 0.650224 | 0.636771 | 0.64574 | 0.928251 | 1 | 0.878924 | 0.627803 | 0.973094 |
| DQ648856.1 | 0.636771 | 0.641256 | 0.632287 | 0.874439 | 0.878924 | 1 | 0.632287 | 0.883408 |
| AY278489.2 | 0.730942 | 0.986547 | 0.73991 | 0.627803 | 0.627803 | 0.632287 | 1 | 0.627803 |
| GQ153540.1 | 0.650224 | 0.636771 | 0.64574 | 0.928251 | 0.973094 | 0.883408 | 0.627803 | 1 |
| MN996532.2 | 0.901345 | 0.748879 | 0.896861 | 0.650224 | 0.650224 | 0.627803 | 0.748879 | 0.650224 |
| KJ473811.1 | 0.641256 | 0.636771 | 0.636771 | 0.878924 | 0.883408 | 0.941704 | 0.627803 | 0.887892 |
| NC_014470.1 | 0.704036 | 0.726457 | 0.713004 | 0.641256 | 0.632287 | 0.654709 | 0.726457 | 0.636771 |
| KC881005.1 | 0.757848 | 0.820628 | 0.7713 | 0.641256 | 0.64574 | 0.641256 | 0.820628 | 0.64574 |
| MK211377.1 | 0.654709 | 0.654709 | 0.650224 | 0.950673 | 0.928251 | 0.896861 | 0.64574 | 0.932735 |
| KJ473816.1 | 0.632287 | 0.632287 | 0.627803 | 0.901345 | 0.914798 | 0.910314 | 0.627803 | 0.910314 |
| MK211376.1 | 0.748879 | 0.955157 | 0.762332 | 0.641256 | 0.64574 | 0.650224 | 0.955157 | 0.64574 |
| AY572034.1 | 0.73991 | 0.96861 | 0.748879 | 0.636771 | 0.636771 | 0.641256 | 0.964126 | 0.636771 |
| KP886809.1 | 0.64574 | 0.641256 | 0.641256 | 0.878924 | 0.878924 | 0.973094 | 0.632287 | 0.887892 |
| MT072864.1 | 0.860987 | 0.744395 | 0.874439 | 0.654709 | 0.659193 | 0.636771 | 0.748879 | 0.64574 |
| KF569996.1 | 0.735426 | 0.941704 | 0.748879 | 0.641256 | 0.64574 | 0.650224 | 0.941704 | 0.64574 |
| JX993987.1 | 0.654709 | 0.650224 | 0.650224 | 0.959641 | 0.928251 | 0.892377 | 0.641256 | 0.932735 |
| MK211378.1 | 0.632287 | 0.632287 | 0.627803 | 0.946188 | 0.932735 | 0.887892 | 0.623318 | 0.93722 |
| MK211374.1 | 0.650224 | 0.636771 | 0.64574 | 0.93722 | 0.977578 | 0.892377 | 0.627803 | 0.96861 |
| KJ473815.1 | 0.654709 | 0.641256 | 0.650224 | 0.941704 | 0.977578 | 0.892377 | 0.632287 | 0.996347 |
| JX993988.1 | 0.636771 | 0.650224 | 0.632287 | 0.919283 | 0.914798 | 0.887892 | 0.641256 | 0.901345 |
| DQ071615.1 | 0.64574 | 0.636771 | 0.641256 | 0.959641 | 0.950673 | 0.892377 | 0.627803 | 0.959641 |
| KT444582.1 | 0.753363 | 0.946188 | 0.766816 | 0.650224 | 0.650224 | 0.654709 | 0.946188 | 0.650224 |
| MZ206298.1 | 0.986547 | 0.730942 | 0.959641 | 0.64574 | 0.650224 | 0.636771 | 0.730942 | 0.650224 |
| KJ473814.1 | 0.650224 | 0.636771 | 0.64574 | 0.93722 | 0.977578 | 0.883408 | 0.627803 | 0.96861 |

| | MN996532.2 | KJ473811.1 | NC_014470.1 | KC881005.1 | MK211377.1 | KJ473816.1 |
|---|---|---|---|---|---|---|
| NC_045512.2 | 0.901345 | 0.641256 | 0.704036 | 0.757848 | 0.654709 | 0.632287 |
| NC_004718.3 | 0.748879 | 0.636771 | 0.726457 | 0.820628 | 0.654709 | 0.632287 |
| MT121216.1 | 0.896861 | 0.636771 | 0.713004 | 0.7713 | 0.650224 | 0.627803 |
| DQ648857.1 | 0.650224 | 0.878924 | 0.641256 | 0.641256 | 0.950673 | 0.901345 |
| GQ153542.1 | 0.650224 | 0.883408 | 0.632287 | 0.64574 | 0.928251 | 0.914798 |
| DQ648856.1 | 0.627803 | 0.941704 | 0.654709 | 0.641256 | 0.896861 | 0.910314 |
| AY278489.2 | 0.748879 | 0.627803 | 0.726457 | 0.820628 | 0.64574 | 0.627803 |
| GQ153540.1 | 0.650224 | 0.887892 | 0.636771 | 0.64574 | 0.932733 | 0.910314 |
| MN996532.2 | 1 | 0.632287 | 0.690583 | 0.757848 | 0.654709 | 0.623318 |
| KJ473811.1 | 0.632287 | 1 | 0.636771 | 0.632287 | 0.892377 | 0.90583 |
| NC_014470.1 | 0.690583 | 0.636771 | 1 | 0.753363 | 0.64574 | 0.632287 |
| KC881005.1 | 0.757848 | 0.632287 | 0.753363 | 1 | 0.650224 | 0.627803 |
| MK211377.1 | 0.654709 | 0.892377 | 0.64574 | 0.650224 | 1 | 0.919283 |
| KJ473816.1 | 0.623318 | 0.90583 | 0.632287 | 0.627803 | 0.919283 | 1 |
| MK211376.1 | 0.753363 | 0.636771 | 0.735426 | 0.852018 | 0.659193 | 0.636771 |
| AY572034.1 | 0.753363 | 0.636771 | 0.730942 | 0.834081 | 0.654709 | 0.627803 |
| KP886809.1 | 0.636771 | 0.950673 | 0.654709 | 0.650224 | 0.896861 | 0.910314 |
| MT072864.1 | 0.874439 | 0.641256 | 0.681614 | 0.757848 | 0.650224 | 0.632287 |
| KF569996.1 | 0.753363 | 0.632287 | 0.726457 | 0.834081 | 0.663677 | 0.636771 |
| JX993987.1 | 0.654709 | 0.892377 | 0.64574 | 0.64874 | 0.964126 | 0.923767 |
| MK211378.1 | 0.632287 | 0.887892 | 0.627803 | 0.627803 | 0.964126 | 0.914798 |
| MK211374.1 | 0.650224 | 0.892377 | 0.636771 | 0.64574 | 0.93722 | 0.923767 |
| KJ473815.1 | 0.654709 | 0.892377 | 0.636771 | 0.650224 | 0.941704 | 0.914798 |
| JX993988.1 | 0.632287 | 0.874439 | 0.632287 | 0.64574 | 0.93722 | 0.914798 |
| DQ071615.1 | 0.64574 | 0.892377 | 0.636771 | 0.641256 | 0.959641 | 0.914798 |
| KT444582.1 | 0.762332 | 0.636771 | 0.73991 | 0.843049 | 0.668161 | 0.641256 |
| MZ206298.1 | 0.896861 | 0.641256 | 0.699552 | 0.737848 | 0.654709 | 0.632287 |
| KJ473814.1 | 0.650224 | 0.887892 | 0.64574 | 0.64574 | 0.932735 | 0.923767 |

| | MK211376.1 | AY572034.1 | KP886809.1 | MT072864.1 | KF569996.1 | JX993987.1 | MK211378.1 | MK211374.1 |
|---|---|---|---|---|---|---|---|---|
| NC_045512.2 | 0.748879 | 0.73991 | 0.64574 | 0.860987 | 0.735426 | 0.654709 | 0.632287 | 0.650224 |
| NC_004718.3 | 0.955157 | 0.96861 | 0.641256 | 0.744395 | 0.941704 | 0.650224 | 0.632287 | 0.636771 |
| MT121216.1 | 0.762332 | 0.748879 | 0.641256 | 0.874439 | 0.748879 | 0.650224 | 0.627803 | 0.64574 |
| DQ648857.1 | 0.641256 | 0.636771 | 0.878924 | 0.654709 | 0.641256 | 0.959641 | 0.946188 | 0.93722 |
| GQ153542.1 | 0.64574 | 0.636771 | 0.878924 | 0.659193 | 0.64574 | 0.928251 | 0.932735 | 0.977578 |
| DQ648856.1 | 0.650224 | 0.641256 | 0.973094 | 0.636771 | 0.650224 | 0.892377 | 0.887892 | 0.892377 |
| AY278489.2 | 0.955157 | 0.964126 | 0.632287 | 0.748879 | 0.941704 | 0.641256 | 0.623318 | 0.627803 |
| GQ153540.1 | 0.64574 | 0.636771 | 0.887892 | 0.64574 | 0.64574 | 0.932735 | 0.93722 | 0.96861 |
| MN996532.2 | 0.753363 | 0.753363 | 0.636771 | 0.874439 | 0.753363 | 0.654709 | 0.632287 | 0.650224 |
| KJ473811.1 | 0.636771 | 0.636771 | 0.950673 | 0.641256 | 0.632287 | 0.892377 | 0.887892 | 0.892377 |
| NC_014470.1 | 0.735426 | 0.730942 | 0.654709 | 0.681614 | 0.726457 | 0.64574 | 0.627803 | 0.636771 |
| KC881005.1 | 0.852018 | 0.834081 | 0.650224 | 0.757848 | 0.834081 | 0.64574 | 0.627803 | 0.64574 |
| MK211377.1 | 0.659193 | 0.654709 | 0.896861 | 0.650224 | 0.663677 | 0.964126 | 0.964126 | 0.93722 |
| KJ473816.1 | 0.636771 | 0.627803 | 0.910314 | 0.632287 | 0.636771 | 0.923767 | 0.914798 | 0.923767 |
| MK211376.1 | 1 | 0.950673 | 0.654709 | 0.73991 | 0.973094 | 0.654709 | 0.636771 | 0.64574 |
| AY572034.1 | 0.950673 | 1 | 0.641256 | 0.744395 | 0.93722 | 0.650224 | 0.632287 | 0.636771 |
| KP886809.1 | 0.654709 | 0.641256 | 1 | 0.641256 | 0.64574 | 0.892377 | 0.887892 | 0.892377 |
| MT072864.1 | 0.73991 | 0.744395 | 0.641256 | 1 | 0.726457 | 0.654709 | 0.641256 | 0.663677 |
| KF569996.1 | 0.973094 | 0.93722 | 0.64574 | 0.726457 | 1 | 0.659193 | 0.641256 | 0.64574 |
| JX993987.1 | 0.654709 | 0.650224 | 0.892377 | 0.654709 | 0.659193 | 1 | 0.946188 | 0.93722 |
| MK211378.1 | 0.636771 | 0.632287 | 0.887892 | 0.641256 | 0.641256 | 0.946188 | 1 | 0.932735 |
| MK211374.1 | 0.64574 | 0.636771 | 0.892377 | 0.663677 | 0.64574 | 0.93722 | 0.932735 | 1 |
| KJ473815.1 | 0.650224 | 0.641256 | 0.892377 | 0.654709 | 0.650224 | 0.941704 | 0.93722 | 0.982063 |
| JX993988.1 | 0.654709 | 0.650224 | 0.887892 | 0.636771 | 0.659193 | 0.93722 | 0.93722 | 0.90583 |
| DQ071615.1 | 0.641256 | 0.636771 | 0.892377 | 0.64574 | 0.64574 | 0.959641 | 0.964126 | 0.950673 |
| KT444582.1 | 0.982063 | 0.941704 | 0.659193 | 0.744395 | 0.964126 | 0.663677 | 0.64574 | 0.650224 |
| MZ206298.1 | 0.744395 | 0.73991 | 0.64574 | 0.860987 | 0.730942 | 0.654709 | 0.632287 | 0.650224 |
| KJ473814.1 | 0.64574 | 0.636771 | 0.887892 | 0.663677 | 0.641256 | 0.932735 | 0.928251 | 0.977578 |

| | KJ473815.1 | JX993988.1 | DQ071615.1 | KT444582.1 | MZ206298.1 | KJ473814.1 |
|---|---|---|---|---|---|---|
| NC_045512.2 | 0.654709 | 0.636771 | 0.64574 | 0.753363 | 0.986547 | 0.650224 |
| NC_004718.3 | 0.641256 | 0.650224 | 0.636771 | 0.946188 | 0.730942 | 0.636771 |
| MT121216.1 | 0.650224 | 0.632287 | 0.641256 | 0.766816 | 0.959641 | 0.64574 |
| DQ648857.1 | 0.941704 | 0.919283 | 0.959641 | 0.650224 | 0.64574 | 0.93722 |
| GQ153542.1 | 0.977578 | 0.914798 | 0.950673 | 0.650224 | 0.650224 | 0.977578 |
| DQ648856.1 | 0.892377 | 0.887892 | 0.892377 | 0.654709 | 0.636771 | 0.883408 |
| AY278489.2 | 0.632287 | 0.641256 | 0.627803 | 0.946188 | 0.730942 | 0.627803 |
| GQ153540.1 | 0.986547 | 0.901345 | 0.959641 | 0.650224 | 0.650224 | 0.96861 |
| MN996532.2 | 0.654709 | 0.632287 | 0.64574 | 0.762332 | 0.896861 | 0.650224 |
| KJ473811.1 | 0.892377 | 0.874439 | 0.892377 | 0.636771 | 0.641256 | 0.887892 |
| NC_014470.1 | 0.636771 | 0.632287 | 0.636771 | 0.73991 | 0.699552 | 0.64574 |
| KC881005.1 | 0.650224 | 0.64574 | 0.641256 | 0.843049 | 0.757848 | 0.64574 |
| MK211377.1 | 0.941704 | 0.93722 | 0.959641 | 0.668161 | 0.654709 | 0.932735 |
| KJ473816.1 | 0.914798 | 0.914798 | 0.914798 | 0.641256 | 0.632287 | 0.923767 |
| MK211376.1 | 0.650224 | 0.654709 | 0.641256 | 0.982063 | 0.744395 | 0.64574 |
| AY572034.1 | 0.641256 | 0.650224 | 0.636771 | 0.941704 | 0.73991 | 0.636771 |
| KP886809.1 | 0.892377 | 0.887892 | 0.892377 | 0.659193 | 0.64574 | 0.887892 |
| MT072864.1 | 0.654709 | 0.636771 | 0.64574 | 0.744395 | 0.860987 | 0.663677 |
| KF569996.1 | 0.650224 | 0.659193 | 0.64574 | 0.964126 | 0.730942 | 0.641256 |
| JX993987.1 | 0.941704 | 0.93722 | 0.959641 | 0.663677 | 0.654709 | 0.932735 |
| MK211378.1 | 0.93722 | 0.93722 | 0.964126 | 0.64574 | 0.632287 | 0.928251 |
| MK211374.1 | 0.982063 | 0.90583 | 0.950673 | 0.650224 | 0.650224 | 0.977578 |
| KJ473815.1 | 1 | 0.901345 | 0.964126 | 0.654709 | 0.654709 | 0.973094 |
| JX993988.1 | 0.901345 | 1 | 0.932735 | 0.663677 | 0.636771 | 0.910314 |
| DQ071615.1 | 0.964126 | 0.932735 | 1 | 0.650224 | 0.64574 | 0.941704 |
| KT444582.1 | 0.654709 | 0.663677 | 0.650224 | 1 | 0.748879 | 0.650224 |
| MZ206298.1 | 0.654709 | 0.636771 | 0.64574 | 0.748879 | 1 | 0.650224 |
| KJ473814.1 | 0.973094 | 0.910314 | 0.941704 | 0.650224 | 0.650224 | 1 |

TABLE 14B
Percent Identity of RBDΔRBM Domains

| | NC_045512.2 | NC_004718.3 | MT121216.1 | DQ648857.1 | GQ153542.1 | DQ648856.1 | AY278489.2 | GQ153540.1 |
|---|---|---|---|---|---|---|---|---|
| NC_045512.2 | 1 | 0.842105 | 0.960526 | 0.802632 | 0.802632 | 0.809211 | 0.842105 | 0.802632 |
| NC_004718.3 | 0.842105 | 1 | 0.855263 | 0.789474 | 0.789474 | 0.802632 | 0.980263 | 0.789474 |
| MT121216.1 | 0.960526 | 0.85$263 | 1 | 0.796053 | 0.796033 | 0.802632 | 0.855263 | 0.796053 |
| DQ648857.1 | 0.802632 | 0.789474 | 0.796053 | 1 | 0.967105 | 0.927632 | 0.776316 | 0.973684 |
| GQ153542.1 | 0.802632 | 0.789474 | 0.796053 | 0.967105 | 1 | 0.921053 | 0.776316 | 0.967105 |
| DQ648856.1 | 0.809211 | 0.802632 | 0.802632 | 0.927632 | 0.921053 | 1 | 0.789474 | 0.934211 |
| AY278489.2 | 0.842105 | 0.980263 | 0.855263 | 0.776316 | 0.776316 | 0.789474 | 1 | 0.776316 |
| GQ153540.1 | 0.802632 | 0.789474 | 0.796053 | 0.973684 | 0.967105 | 0.934211 | 0.776316 | 1 |
| MN996532.2 | 0.967105 | 0.855263 | 0.960526 | 0.802632 | 0.802632 | 0.809211 | 0.855263 | 0.802632 |
| KJ473811.1 | 0.802632 | 0.796053 | 0.796053 | 0.914474 | 0.907895 | 0.947368 | 0.782895 | 0.921053 |
| NC_014470.1 | 0.835526 | 0.842105 | 0.848684 | 0.789474 | 0.782895 | 0.802632 | 0.842105 | 0.789474 |
| KC881005.1 | 0.855263 | 0.953947 | 0.875 | 0.796053 | 0.796053 | 0.809211 | 0.953947 | 0.796053 |
| MK211377.1 | 0.815789 | 0.802632 | 0.809211 | 0.973684 | 0.967105 | 0.947368 | 0.789474 | 0.980263 |
| KJ473816.1 | 0.789474 | 0.782895 | 0.782895 | 0.940789 | 0.940789 | 0.921053 | 0.776316 | 0.940789 |
| MK211376.1 | 0.855263 | 0.960526 | 0.875 | 0.796053 | 0.796053 | 0.809211 | 0.960526 | 0.796053 |
| AY572034.1 | 0.855263 | 0.986842 | 0.868421 | 0.789474 | 0.789474 | 0.802632 | 0.980263 | 0.789474 |
| KP886809.1 | 0.809211 | 0.802632 | 0.802632 | 0.927632 | 0.914474 | 0.993421 | 0.789474 | 0.927632 |
| MT072864.1 | 0.914474 | 0.861842 | 0.927632 | 0.796053 | 0.796053 | 0.789474 | 0.868421 | 0.782895 |
| KF569996.1 | 0.842105 | 0.947368 | 0.861842 | 0.789474 | 0.796053 | 0.809211 | 0.947368 | 0.796053 |
| JX993987.1 | 0.815789 | 0.802632 | 0.809211 | 0.973684 | 0.967105 | 0.947368 | 0.789474 | 0.980263 |
| MK211378.1 | 0.796053 | 0.782895 | 0.789474 | 0.973684 | 0.967105 | 0.907632 | 0.769737 | 0.980263 |
| MK211374.1 | 0.809211 | 0.796053 | 0.802632 | 0.980263 | 0.973684 | 0.934211 | 0.782895 | 0.967105 |
| KJ473815.1 | 0.809211 | 0.796053 | 0.802632 | 0.986842 | 0.967105 | 0.940789 | 0.782895 | 0.986842 |
| JX993988.1 | 0.789474 | 0.802632 | 0.782895 | 0.927632 | 0.947368 | 0.927632 | 0.789474 | 0.934211 |
| DQ071615.1 | 0.802632 | 0.789474 | 0.796053 | 0.980263 | 0.973684 | 0.934211 | 0.776316 | 0.993421 |
| KT444582.1 | 0.861842 | 0.953947 | 0.881579 | 0.802632 | 0.802632 | 0.815789 | 0.953947 | 0.802632 |
| MZ206298.1 | 0.993421 | 0.842105 | 0.960526 | 0.802632 | 0.802632 | 0.809211 | 0.842105 | 0.802632 |
| KJ473814.1 | 0.809211 | 0.796053 | 0.802632 | 0.980263 | 0.973684 | 0.927632 | 0.782895 | 0.967105 |

| | MN996532.2 | KJ473811.1 | NC_014470.1 | KC881005.1 | MK211377.1 | KJ473816.1 |
|---|---|---|---|---|---|---|
| NC_045512.2 | 0.967105 | 0.802632 | 0.835526 | 0.855263 | 0.815789 | 0.789474 |
| NC_004718.3 | 0.855263 | 0.796053 | 0.842105 | 0.953947 | 0.802632 | 0.782895 |
| MT121216.1 | 0.960526 | 0.796053 | 0.848684 | 0.875 | 0.809211 | 0.782895 |
| DQ648857.1 | 0.802632 | 0.914474 | 0.789474 | 0.796053 | 0.973684 | 0.940759 |
| GQ153542.1 | 0.802632 | 0.907895 | 0.782895 | 0.796053 | 0.967105 | 0.940789 |
| DQ648356.1 | 0.809211 | 0.947368 | 0.802632 | 0.809211 | 0.947368 | 0.921053 |
| AY278489.2 | 0.855263 | 0.782895 | 0.842105 | 0.953947 | 0.759474 | 0.776316 |
| GQ153540.1 | 0.802632 | 0.921053 | 0.789474 | 0.796053 | 0.980263 | 0.940789 |
| MN996532.2 | 1 | 0.802632 | 0.842105 | 0.875 | 0.815789 | 0.789474 |
| KJ473811.1 | 0.802632 | 1 | 0.776316 | 0.789474 | 0.927632 | 0.901316 |
| NC_014470.1 | 0.842105 | 0.776316 | 1 | 0.861842 | 0.796053 | 0.776316 |
| KC881005.1 | 0.875 | 0.789474 | 0.861842 | 1 | 0.809211 | 0.782895 |
| MK211377.1 | 0.815739 | 0.927632 | 0.796053 | 0.809211 | 1 | 0.947368 |
| KJ473816.1 | 0.789474 | 0.901316 | 0.776316 | 0.782895 | 0.947368 | 1 |
| MK211376.1 | 0.875 | 0.789474 | 0.861842 | 0.993421 | 0.809211 | 0.782895 |
| AY572034.1 | 0.868421 | 0.796053 | 0.855263 | 0.967105 | 0.802632 | 0.776316 |
| KP886809.1 | 0.809211 | 0.947368 | 0.802632 | 0.815789 | 0.940789 | 0.914474 |
| MT072864.1 | 0.927632 | 0.782895 | 0.835526 | 0.868421 | 0.796053 | 0.782895 |
| KF569996.1 | 0.861842 | 0.782895 | 0.848684 | 0.973684 | 0.809211 | 0.782895 |
| JX993987.1 | 0.815789 | 0.927632 | 0.796053 | 0.809211 | 0.993421 | 0.947368 |
| MK211378.1 | 0.796053 | 0.914474 | 0.776316 | 0.789474 | 0.973684 | 0.940759 |
| MK211374.1 | 0.809211 | 0.914474 | 0.789474 | 0.802632 | 0.980263 | 0.947368 |
| KJ473815.1 | 0.809211 | 0.921053 | 0.789474 | 0.802632 | 0.986842 | 0.940789 |
| JX993988.1 | 0.789474 | 0.894737 | 0.776316 | 0.809211 | 0.934211 | 0.927632 |
| DQ071615.1 | 0.802632 | 0.921053 | 0.782893 | 0.796053 | 0.980263 | 0.934211 |
| KT444582.1 | 0.881579 | 0.789474 | 0.868423 | 0.993421 | 0.815789 | 0.789474 |
| MZ206298.1 | 0.960526 | 0.802632 | 0.835526 | 0.855263 | 0.815789 | 0.789474 |
| KJ473814.1 | 0.809211 | 0.914474 | 0.802632 | 0.802632 | 0.973684 | 0.953947 |

| | MK211376.1 | AY572034.1 | KP886809.1 | MT072864.1 | KF569996.1 | JX993987.1 | MK211378.1 | MK211374.1 |
|---|---|---|---|---|---|---|---|---|
| NC_045512.2 | 0.833263 | 0.855263 | 0.809211 | 0.914474 | 0.842105 | 0.813789 | 0.796033 | 0.809211 |
| NC_004718.3 | 0.960526 | 0.986842 | 0.802632 | 0.861842 | 0.947368 | 0.802632 | 0.782895 | 0.796053 |
| MT121216.1 | 0.875 | 0.868421 | 0.802632 | 0.927632 | 0.861842 | 0.809211 | 0.789474 | 0.802632 |
| DQ648857.1 | 0.796053 | 0.789474 | 0.927632 | 0.796053 | 0.789474 | 0.973684 | 0.973684 | 0.980263 |
| GQ153542.1 | 0.796053 | 0.789474 | 0.914474 | 0.796053 | 0.796033 | 0.967105 | 0.967105 | 0.973684 |
| DQ648856.1 | 0.809211 | 0.802632 | 0.993421 | 0.789474 | 0.809211 | 0.947368 | 0.927632 | 0.934211 |
| AY278489.2 | 0.960526 | 0.980263 | 0.789474 | 0.868421 | 0.947368 | 0.789474 | 0.769737 | 0.782893 |
| GQ133340.1 | 0.796053 | 0.789474 | 0.927632 | 0.782895 | 0.796053 | 0.980263 | 0.980263 | 0.967103 |
| MN996532.2 | 0.875 | 0.868421 | 0.809211 | 0.927632 | 0.861842 | 0.815789 | 0.796053 | 0.809211 |
| KJ473811.1 | 0.789474 | 0.796033 | 0.947368 | 0.782895 | 0.782895 | 0.927632 | 0.914474 | 0.914474 |
| NC_014470.1 | 0.861842 | 0.833263 | 0.802632 | 0.835526 | 0.848684 | 0.796053 | 0.776316 | 0.789474 |
| KC881005.1 | 0.993421 | 0.967105 | 0.815789 | 0.868421 | 0.973684 | 0.809211 | 0.789474 | 0.802632 |
| MK211377.1 | 0.809211 | 0.802632 | 0.940789 | 0.796053 | 0.809211 | 0.993421 | 0.973684 | 0.980263 |
| KJ473816.1 | 0.782895 | 0.776316 | 0.914474 | 0.782895 | 0.782895 | 0.947368 | 0.940789 | 0.947368 |
| MK211376.1 | 1 | 0.973684 | 0.815789 | 0.866421 | 0.980263 | 0.809211 | 0.789474 | 0.802632 |
| AY572034.1 | 0.973684 | 1 | 0.802632 | 0.875 | 0.960526 | 0.802632 | 0.782895 | 0.796053 |
| KP886809.1 | 0.815789 | 0.802632 | 1 | 0.789474 | 0.802632 | 0.940789 | 0.921053 | 0.927632 |
| MT072864.1 | 0.868421 | 0.875 | 0.789474 | 1 | 0.856263 | 0.802632 | 0.789474 | 0.802632 |
| KF569996.1 | 0.980263 | 0.960526 | 0.802632 | 0.855263 | 1 | 0.809211 | 0.789474 | 0.802632 |
| JX993987.1 | 0.809211 | 0.802632 | 0.940789 | 0.802632 | 0.809211 | 1 | 0.967105 | 0.980263 |
| MK211378.1 | 0.789474 | 0.782895 | 0.921053 | 0.789474 | 0.789474 | 0.967105 | 1 | 0.967105 |
| MK211374.1 | 0.802632 | 0.796053 | 0.927632 | 0.802632 | 0.802632 | 0.980263 | 0.967105 | 1 |
| KJ473815.1 | 0.802632 | 0.796053 | 0.934211 | 0.789474 | 0.802632 | 0.986842 | 0.973684 | 0.980263 |
| JX993988.1 | 0.809211 | 0.802632 | 0.921053 | 0.782893 | 0.809211 | 0.934211 | 0.940789 | 0.934211 |
| DQ071615.1 | 0.796053 | 0.789474 | 0.927632 | 0.782895 | 0.796053 | 0.980263 | 0.980263 | 0.973684 |
| KT444582.1 | 0.993421 | 0.967105 | 0.822368 | 0.375 | 0.973684 | 0.815789 | 0.796053 | 0.809211 |
| MZ206298.1 | 0.855063 | 0.833263 | 0.809211 | 0.914474 | 0.842105 | 0.815789 | 0.796053 | 0.809211 |
| KJ473814.1 | 0.802632 | 0.796053 | 0.927632 | 0.802632 | 0.796053 | 0.973684 | 0.960526 | 0.973684 |

| | KJ473815.1 | JX993988.1 | DQ071615.1 | KT444582.1 | MZ206298.1 | KJ473814.1 |
|---|---|---|---|---|---|---|
| NC_045512.2 | 0.809211 | 0.789474 | 0.802632 | 0.861842 | 0.993421 | 0.809211 |
| NC_004718.3 | 0.796053 | 0.802632 | 0.789474 | 0.953947 | 0.842103 | 0.796053 |
| MT121216.1 | 0.802632 | 0.782895 | 0.796053 | 0.881579 | 0.960526 | 0.802632 |
| DQ648857.1 | 0.986842 | 0.927632 | 0.980263 | 0.802632 | 0.802632 | 0.980263 |
| GQ153542.1 | 0.967105 | 0.947368 | 0.973684 | 0.802632 | 0.802632 | 0.973684 |
| DQ648856.1 | 0.940789 | 0.927632 | 0.934211 | 0.815789 | 0.809211 | 0.927632 |
| AY278489.2 | 0.782895 | 0.789474 | 0.776316 | 0.953947 | 0.842105 | 0.782895 |
| GQ153540.1 | 0.986842 | 0.934211 | 0.993421 | 0.802632 | 0.802632 | 0.967105 |
| MN996532.2 | 0.809211 | 0.789474 | 0.802632 | 0.881579 | 0.960526 | 0.809211 |
| KJ473811.1 | 0.921053 | 0.894737 | 0.921053 | 0.789474 | 0.802632 | 0.914474 |
| NC_014470.1 | 0.789474 | 0.776316 | 0.782895 | 0.868421 | 0.835526 | 0.802632 |
| KC881005.1 | 0.802632 | 0.809211 | 0.796053 | 0.993421 | 0.855263 | 0.802632 |
| MK211377.1 | 0.986842 | 0.934211 | 0.980263 | 0.815789 | 0.815789 | 0.973684 |
| KJ473816.1 | 0.940789 | 0.927632 | 0.934211 | 0.789474 | 0.789474 | 0.953947 |
| MK211376.1 | 0.802632 | 0.809211 | 0.796053 | 0.993421 | 0.855263 | 0.802632 |
| AY572034.1 | 0.796053 | 0.802632 | 0.789474 | 0.967105 | 0.855263 | 0.796063 |
| KP886809.1 | 0.934211 | 0.921053 | 0.927632 | 0.822368 | 0.809211 | 0.927632 |
| MT072864.1 | 0.789474 | 0.782895 | 0.782895 | 0.875 | 0.914474 | 0.802632 |
| KF569996.1 | 0.802632 | 0.809211 | 0.796053 | 0.973684 | 0.842105 | 0.796053 |
| JX993987.1 | 0.986842 | 0.934211 | 0.980263 | 0.815789 | 0.815789 | 0.973684 |
| MK211378.1 | 0.973684 | 0.940789 | 0.980263 | 0.796053 | 0.796053 | 0.960326 |
| MK211374.1 | 0.980263 | 0.934211 | 0.973684 | 0.809211 | 0.809211 | 0.973684 |
| KJ473815.1 | 1 | 0.927632 | 0.993421 | 0.809211 | 0.809211 | 0.967105 |
| JX993988.1 | 0.927632 | 1 | 0.934211 | 0.815789 | 0.789474 | 0.940789 |
| DQ071615.1 | 0.993421 | 0.934211 | 1 | 0.802632 | 0.802632 | 0.960526 |
| KT444582.1 | 0.809211 | 0.815789 | 0.802632 | 1 | 0.861842 | 0.809211 |
| MZ206298.1 | 0.809211 | 0.789474 | 0.802632 | 0.861842 | 1 | 0.809211 |
| KJ473814.1 | 0.967105 | 0.940789 | 0.960526 | 0.809211 | 0.809211 | 1 |

TABLE 14C
Percent Identity of RBM Domains

| | NC_045512.2 | NC_004718.3 | MT121216.1 | DQ648857.1 | GQ153542.1 | DQ648856.1 | AY278489.2 |
|---|---|---|---|---|---|---|---|
| NC_045512.2 | 1 | 0.492958 | 0.985915 | 0.309859 | 0.323944 | 0.267606 | 0.492958 |
| NC_004718.3 | 0.492958 | 1 | 0.492958 | 0.309859 | 0.309859 | 0.295775 | 1 |
| MT121216.1 | 0.985915 | 0.492958 | 1 | 0.309859 | 0.323944 | 0.267606 | 0.492958 |
| DQ648857.1 | 0.309859 | 0.309859 | 0.309839 | 1 | 0.84507 | 0.760563 | 0.309859 |
| GQ153542.1 | 0.323944 | 0.309859 | 0.323944 | 0.84507 | 1 | 0.788732 | 0.309859 |
| DQ648856.1 | 0.267606 | 0.295775 | 0.267606 | 0.760563 | 0.788732 | 1 | 0.295775 |
| AY278489.2 | 0.492938 | 1 | 0.492958 | 0.309859 | 0.309859 | 0.295775 | 1 |
| GQ153540.1 | 0.323944 | 0.309859 | 0.323944 | 0.830986 | 0.985915 | 0.774648 | 0.309859 |
| MN996532.2 | 0.760563 | 0.521127 | 0.760563 | 0.323944 | 0.323944 | 0.239437 | 0.521127 |
| KJ473811.1 | 0.295775 | 0.295775 | 0.295775 | 0.802817 | 0.830986 | 0.929577 | 0.295775 |
| NC_014470.1 | 0.422535 | 0.478873 | 0.422535 | 0.323944 | 0.309859 | 0.338028 | 0.478873 |
| KC881005.1 | 0.549296 | 0.535211 | 0.549296 | 0.309859 | 0.323944 | 0.28169 | 0.535211 |
| MK211377.1 | 0.309859 | 0.338028 | 0.309859 | 0.901408 | 0.84507 | 0.788732 | 0.338028 |
| KJ473816.1 | 0.295775 | 0.309859 | 0.295775 | 0.816901 | 0.839155 | 0.887324 | 0.309859 |
| MK211376.1 | 0.521127 | 0.943662 | 0.521127 | 0.309859 | 0.323944 | 0.309859 | 0.943662 |
| AY572034.1 | 0.492958 | 0.929577 | 0.492958 | 0.309859 | 0.309859 | 0.295775 | 0.929577 |
| KP886809.1 | 0.295775 | 0.295775 | 0.295775 | 0.774648 | 0.802817 | 0.929577 | 0.295775 |
| MT072864.1 | 0.746479 | 0.492958 | 0.760563 | 0.352113 | 0.366197 | 0.309859 | 0.492958 |
| KF569996.1 | 0.507042 | 0.929577 | 0.507042 | 0.323944 | 0.323944 | 0.309859 | 0.929577 |
| JX993987.1 | 0.309859 | 0.323944 | 0.309859 | 0.929577 | 0.84507 | 0.774648 | 0.323944 |
| MK211378.1 | 0.28169 | 0.309859 | 0.28169 | 0.887324 | 0.859155 | 0.802817 | 0.309859 |
| MK211374.1 | 0.309859 | 0.295775 | 0.309859 | 0.84507 | 0.985915 | 0.802817 | 0.295775 |
| KJ473815.1 | 0.323944 | 0.309859 | 0.323944 | 0.84507 | 1 | 0.788732 | 0.309859 |
| JX993988.1 | 0.309859 | 0.323944 | 0.309859 | 0.901408 | 0.84507 | 0.802817 | 0.323944 |
| DQ071615.1 | 0.309859 | 0.309859 | 0.309859 | 0.915493 | 0.901408 | 0.802817 | 0.309859 |
| KT444582.1 | 0.521127 | 0.929577 | 0.521127 | 0.323944 | 0.323944 | 0.309859 | 0.929577 |
| MZ206298.1 | 0.971831 | 0.492958 | 0.957746 | 0.309859 | 0.323944 | 0.267606 | 0.492958 |
| KJ473814.1 | 0.309859 | 0.295775 | 0.309859 | 0.84507 | 0.985915 | 0.788732 | 0.295775 |

| | GQ153540.1 | MN996532.2 | KJ473811.1 | NC_014470.1 | KC881005.1 | MK211377.1 | KJ473816.1 |
|---|---|---|---|---|---|---|---|
| NC_045512.2 | 0.323944 | 0.760563 | 0.295775 | 0.422535 | 0.549296 | 0.309859 | 0.295775 |
| NC_004718.3 | 0.309859 | 0.521127 | 0.295775 | 0.478873 | 0.535211 | 0.338028 | 0.309859 |
| MT121216.1 | 0.3239444 | 0.760563 | 0.295775 | 0.422535 | 0.549296 | 0.309859 | 0.295775 |
| DQ648857.1 | 0.830986 | 0.323944 | 0.802817 | 0.323944 | 0.309859 | 0.901408 | 0.816901 |
| GQ153542.1 | 0.985915 | 0.323944 | 0.830986 | 0.309859 | 0.323944 | 0.84507 | 0.859155 |
| DQ648856.1 | 0.774648 | 0.239437 | 0.929577 | 0.338028 | 0.28169 | 0.788732 | 0.887324 |
| AY278489.2 | 0.309859 | 0.521127 | 0.295775 | 0.478873 | 0.535211 | 0.338028 | 0.309859 |
| GQ153540.1 | 1 | 0.323944 | 0.816901 | 0.309859 | 0.323944 | 0.830986 | 0.84507 |
| MN996532.2 | 0.323944 | 1 | 0.267606 | 0.366197 | 0.507042 | 0.309859 | 0.267606 |
| KJ473811.1 | 0.816901 | 0.267606 | 1 | 0.338028 | 0.295775 | 0.816901 | 0.915493 |
| NC_014470.1 | 0.309859 | 0.366197 | 0.338028 | 1 | 0.521127 | 0.323944 | 0.323944 |
| KC881005.1 | 0.323944 | 0.307042 | 0.295775 | 0.521127 | 1 | 0.309859 | 0.295775 |
| MK211377.1 | 0.830986 | 0.309859 | 0.816901 | 0.323944 | 0.309859 | 1 | 0.859155 |
| KJ473816.1 | 0.84507 | 0.267606 | 0.915493 | 0.323944 | 0.295775 | 0.859155 | 1 |
| MK211376.1 | 0.323944 | 0.492958 | 0.309859 | 0.464789 | 0.549296 | 0.338028 | 0.323944 |
| AY572034.1 | 0.309859 | 0.507042 | 0.295775 | 0.464789 | 0.549296 | 0.338028 | 0.309859 |
| KP886809.1 | 0.802817 | 0.267606 | 0.937746 | 0.338028 | 0.295775 | 0.802817 | 0.901408 |
| MT072864.1 | 0.352113 | 0.760563 | 0.338028 | 0.352113 | 0.521127 | 0.338028 | 0.309859 |
| KF569996.1 | 0.323944 | 0.521127 | 0.309859 | 0.464789 | 0.535211 | 0.352113 | 0.323944 |
| JX993987.1 | 0.830986 | 0.309859 | 0.816901 | 0.323944 | 0.295775 | 0.901408 | 0.873239 |
| MK211378.1 | 0.84507 | 0.28169 | 0.830986 | 0.309859 | 0.28169 | 0.943662 | 0.859155 |
| MK211374.1 | 0.971831 | 0.309859 | 0.84507 | 0.309859 | 0.309859 | 0.84507 | 0.873239 |
| KJ473815.1 | 0.985915 | 0.323944 | 0.830986 | 0.309859 | 0.323944 | 0.84507 | 0.859155 |
| JX993988.1 | 0.830986 | 0.295775 | 0.830986 | 0.323944 | 0.295775 | 0.943662 | 0.887324 |
| DQ071615.1 | 0.887324 | 0.309859 | 0.830986 | 0.323944 | 0.309859 | 0.915493 | 0.873239 |
| KT444582.1 | 0.323944 | 0.507042 | 0.309859 | 0.464789 | 0.521127 | 0.352113 | 0.323944 |
| MZ206298.1 | 0.323944 | 0.760563 | 0.295775 | 0.408451 | 0.549296 | 0.309859 | 0.295775 |
| KJ473814.1 | 0.971831 | 0.309859 | 0.830986 | 0.309859 | 0.309859 | 0.84507 | 0.859155 |

| | MK211376.1 | AY572034.1 | KP886809.1 | MT072864.1 | KF569996.1 | JX993987.1 | MK211378.1 |
|---|---|---|---|---|---|---|---|
| NC_045512.2 | 0.521127 | 0.492958 | 0.295775 | 0.746479 | 0.507042 | 0.309859 | 0.28169 |
| NC_004718.3 | 0.943662 | 0.929377 | 0.295775 | 0.492958 | 0.929577 | 0.323944 | 0.309859 |
| MT121216.1 | 0.521127 | 0.492958 | 0.295775 | 0.760563 | 0.507042 | 0.309859 | 0.28169 |
| DQ648857.1 | 0.309859 | 0.309859 | 0.774648 | 0.352113 | 0.323944 | 0.929577 | 0.887324 |
| GQ153542.1 | 0.323944 | 0.309S39 | 0.802817 | 0.366197 | 0.323944 | 0.84507 | 0.859155 |
| DQ648856.1 | 0.309859 | 0.295775 | 0.929577 | 0.309859 | 0.309839 | 0.774648 | 0.802817 |
| AY278489.2 | 0.943662 | 0.929577 | 0.295775 | 0.492958 | 0.929577 | 0.323944 | 0.309859 |
| GQ153540.1 | 0.323944 | 0.309859 | 0.802817 | 0.352113 | 0.323944 | 0.830986 | 0.84507 |
| MN996532.2 | 0.492958 | 0.507042 | 0.267606 | 0.760563 | 0.521127 | 0.309859 | 0.28169 |
| KJ473811.1 | 0.309859 | 0.295775 | 0.957746 | 0.338028 | 0.309859 | 0.816901 | 0.830986 |
| NC_014470.1 | 0.464789 | 0.464789 | 0.338028 | 0.352113 | 0.464789 | 0.323944 | 0.309859 |
| KC881005.1 | 0.349296 | 0.549296 | 0.295775 | 0.521127 | 0.535211 | 0.295775 | 0.28169 |
| MK211377.1 | 0.338028 | 0.338028 | 0.802817 | 0.338028 | 0.352113 | 0.901408 | 0.943662 |
| KJ473816.1 | 0.323944 | 0.309859 | 0.901408 | 0.309859 | 0.323944 | 0.873239 | 0.859155 |
| MK211376.1 | 1 | 0.901408 | 0.309859 | 0.464789 | 0.957746 | 0.323944 | 0.309859 |
| AY572034.1 | 0.901408 | 1 | 0.295775 | 0.464789 | 0.887324 | 0.323944 | 0.309859 |
| KP886809.1 | 0.309859 | 0.295775 | 1 | 0.323944 | 0.309859 | 0.788732 | 0.816901 |
| MT072864.1 | 0.464789 | 0.464789 | 0.323944 | 1 | 0.450704 | 0.338028 | 0.323944 |
| KF569996.1 | 0.957746 | 0.887324 | 0.309859 | 0.450704 | 1 | 0.338028 | 0.323944 |
| JX993987.1 | 0.323944 | 0.323944 | 0.788732 | 0.338028 | 0.338028 | 1 | 0.901408 |
| MK211378.1 | 0.309859 | 0.309859 | 0.816901 | 0.323944 | 0.323944 | 0.901408 | 1 |
| MK211374.1 | 0.309839 | 0.295775 | 0.816901 | 0.366197 | 0.309859 | 0.84507 | 0.859155 |
| KJ473815.1 | 0.323944 | 0.309859 | 0.802817 | 0.366197 | 0.323944 | 0.84507 | 0.859155 |
| JX993988.1 | 0.323944 | 0.323944 | 0.816901 | 0.323944 | 0.338028 | 0.943662 | 0.929577 |
| DQ071615.1 | 0.309839 | 0.309859 | 0.816901 | 0.332113 | 0.323944 | 0.915493 | 0.929577 |
| KT444582.1 | 0.957746 | 0.887324 | 0.309859 | 0.464789 | 0.943662 | 0.338028 | 0.323944 |
| MZ206298.1 | 0.507042 | 0.492958 | 0.295775 | 0.746479 | 0.492958 | 0.309859 | 0.28169 |
| KJ473814.1 | 0.309859 | 0.295775 | 0.802817 | 0.366197 | 0.309859 | 0.84507 | 0.859155 |

| | MK211374.1 | KJ473815.1 | JX993988.1 | DQ071615.1 | KT444582.1 | MZ206298.1 | KJ473814.1 |
|---|---|---|---|---|---|---|---|
| NC_045512.2 | 0.309859 | 0.323944 | 0.309859 | 0.309859 | 0.521127 | 0.971831 | 0.309859 |
| NC_004718.3 | 0.295775 | 0.309859 | 0.323944 | 0.309859 | 0.929577 | 0.492958 | 0.295775 |
| MT121216.1 | 0.309859 | 0.323944 | 0.309859 | 0.309859 | 0.521127 | 0.957746 | 0.309859 |
| DQ648857.1 | 0.84507 | 0.84507 | 0.901408 | 0.915493 | 0.323944 | 0.309859 | 0.84507 |
| GQ153542.1 | 0.985915 | 1 | 0.84507 | 0.901408 | 0.323944 | 0.323944 | 0.985915 |
| DQ648856.1 | 0.802817 | 0.788732 | 0.802817 | 0.802817 | 0.309839 | 0.267606 | 0.788732 |
| AY278489.2 | 0.295775 | 0.309859 | 0.323944 | 0.309859 | 0.929577 | 0.492958 | 0.295775 |
| GQ153540.1 | 0.971831 | 0.985915 | 0.830986 | 0.887324 | 0.323944 | 0.323944 | 0.971831 |
| MN996532.2 | 0.309859 | 0.323944 | 0.295775 | 0.309859 | 0.507042 | 0.760563 | 0.309859 |
| KJ473811.1 | 0.84507 | 0.830986 | 0.830986 | 0.830986 | 0.309859 | 0.295775 | 0.830986 |
| NC_014470.1 | 0.309859 | 0.309859 | 0.323944 | 0.323944 | 0.464789 | 0.408451 | 0.309859 |
| KC881005.1 | 0.309859 | 0.323944 | 0.295775 | 0.309859 | 0.521127 | 0.549296 | 0.309859 |
| MK211377.1 | 0.84507 | 0.84507 | 0.943662 | 0.915493 | 0.352113 | 0.309859 | 0.84507 |
| KJ473816.1 | 0.873239 | 0.859155 | 0.887324 | 0.873239 | 0.323944 | 0.295775 | 0.859155 |
| MK211376.1 | 0.309859 | 0.323944 | 0.323944 | 0.309859 | 0.957746 | 0.507042 | 0.309859 |
| AY572034.1 | 0.295775 | 0.309859 | 0.323944 | 0.309859 | 0.887324 | 0.492958 | 0.295775 |
| KP886809.1 | 0.816901 | 0.802817 | 0.816901 | 0.816901 | 0.309859 | 0.295775 | 0.802817 |
| MT072864.1 | 0.366197 | 0.366197 | 0.323944 | 0.352113 | 0.464789 | 0.746479 | 0.366197 |
| KF569996.1 | 0.309859 | 0.323944 | 0.338028 | 0.323944 | 0.943662 | 0.492958 | 0.309859 |
| JX993987.1 | 0.84507 | 0.84507 | 0.943662 | 0.915493 | 0.338028 | 0.309859 | 0.84507 |
| MK211378.1 | 0.859155 | 0.839155 | 0.929577 | 0.929577 | 0.323944 | 0.28169 | 0.859155 |
| MK211374.1 | 1 | 0.985915 | 0.84507 | 0.901408 | 0.309859 | 0.309859 | 0.985913 |
| KJ473815.1 | 0.985915 | 1 | 0.84507 | 0.901408 | 0.323944 | 0.323944 | 0.988913 |
| JX993988.1 | 0.84507 | 0.84507 | 1 | 0.929577 | 0.338028 | 0.309859 | 0.84507 |
| DQ071615.1 | 0.901408 | 0.901408 | 0.929577 | 1 | 0.323944 | 0.309859 | 0.901408 |
| KT444582.1 | 0.309859 | 0.323944 | 0.338028 | 0.323944 | 1 | 0.507042 | 0.309859 |
| MZ206298.1 | 0.309859 | 0.323944 | 0.309859 | 0.309859 | 0.507042 | 1 | 0.309859 |
| KJ473814.1 | 0.985913 | 0.985915 | 0.84507 | 0.901408 | 0.309859 | 0.309859 | 1 |

### TCE Cassette Design

Four unique T-cell epitope (TCE) cassette candidates were generated that contained conserved CD8+ T-cell epitopes derived from multiple SARS-COV-2 proteins other than Spike. The selected epitopes were 100% conserved across 75 Sarbecovirus strains and were sourced from either validated epitopes [Saini et al, 2021 (Sci Immunol. 2021 Apr. 14; 6(58): eabf7550); Tarke et al, 2021 (Cell Rep Med. 2021 Feb. 16; 2(2):100204.)] or predicted using Gritstone's EDGE™ epitope prediction platform [Bulik-Sullivan et al, 2018 (Nat Biotechnol. 2018 Dec. 17)] further modified to predict viral epitopes. The validated T-cell epitopes made up a 271 AA cassette sequence. To maximize the coverage of the vaccine across populations, the T-cell cassette design aimed to have most (>95%) individuals across 4 major ancestry groups (European, Asian, Hispanic and African descent) receive at least 1 validated CD8+ T-cell epitope or 8 predicted epitopes within a desired cassette sequence footprint size. The epitope frames in the resulting candidate antigen cassettes were arranged to reduce the likelihood of immune responses against junctional epitopes. Finally, all the cassettes were codon optimized to enhance expression in mammalian cells. A summary of the TCE cassettes designed, referred to as TCE12 to 15, is shown below in Table 15. The epitopes and order encoded by each of cassettes TCE12 to 15 are shown below in Tables 16A to D, respectively.

TABLE 15
Design of TCE Cassettes

| | TCE12 | TCE13 | TCE14 | TCE15 |
|---|---|---|---|---|
| Size | 990 AA | 781 AA | 598 AA | 385 AA |
| Source | Literature & | Literature & | Literature & | Literature & |
| | EDGE | EDGE | EDGE | EDGE |
| Protein | NSP12, NSP13, | NSP12, NSP13, | NSP12, NSP13, | NSP12, NSP13, |
| targets | NSP8, NSP14, N, | NSP8, NSP14, N, | NSP8, NSP14, N, | NSP14, N, NSP3 |
| | NSP5, NSP3, | NSP5, NSP3, M | NSP5, NSP3 & M | & M |
| | NSP9, M, NSP7 | | | |
| | & NSP4 | | | |
| % WT | 39%, 29%, 35%, | 34%, 19%, 35%, | 31%, 13%, 13%, | 27%, 5%, 3%, |
| Full | 26%, 10%, 22%, | 26%, 10%, 12%, | 12%, 10%, 14%, | 10%, 1%, 15% |
| Length | 2%, 20%, 15%, | 2%, 15% | 1%, 15% | |
| Protein | 22%, 5% | | | |

TABLE 16A
TCE12 Cassette

| | | Start | End | SEQ ID | |
|---|---|---|---|---|---|
| Frame | Gene | aa | aa | NO | Amino Acid Sequence |
| 1 | Nsp12 | 4414 | 4431 | | GTGTSTDVVYRAFDIYN (SEQ ID NO: 27974) |
| 2 | Nsp12 | 4657 | 4676 | | IKWDLLKYDFTEERLKLFD (SEQ ID NO: 27975) |
| 3 | Nsp12 | 4705 | 4745 | | NVLFSTVFPPTSFGPLVRKIFVDGVPFVVSTGYHFRELGV (SEQ |
| | | | | | ID NO: 27976) |
| 4 | Nsp12 | 4751 | 4785 | | NLHSSRLSFKELLVYAADPAMHAASGNLLLDKRT (SEQ ID |
| | | | | | NO: 27977) |
| 5 | Nsp12 | 4814 | 4871 | | AVSKGFFKEGSSVELKHFFFAQDGNAAISDYDYYRYNLPTMC |
| | | | | | DIRQLLFVVEVVDKY (SEQ ID NO: 27978) |
| 6 | Nsp12 | 4878 | 4908 | | CINANQVIVNNLDKSAGFPFNKWGKARLYY (SEQ ID |
| | | | | | NO: 27979) |
| 7 | Nsp12 | 4948 | 4994 | | VAGVSICSTMTNRQFHQKLLKSIAATRGATVVIGTSKFYGGW |
| | | | | | HNML (SEQ ID NO: 27980) |
| 8 | Nsp12 | 5132 | 5154 | | FVNEFYAYLRKHFSMMILSDDA (SEQ ID NO: 27981) |
| 9 | Nsp12 | 5208 | 5235 | | TMLVKQGDDYVYLPYPDPSRILGAGCF (SEQ ID NO: 27982) |
| 10 | Nsp12 | 5241 | 5296 | | TDGTLMIERFVSLAIDAYPLTKHPNQEYADVFHLYLQYIRKL |
| | | | | | HDELTGHMLDMYS (SEQ ID NO: 27983) |
| 11 | Nsp12 | 5303 | 5320 | | TSRYWEPEFYEAMYTPH (SEQ ID NO: 27984) |
| 12 | Nsp13 | 5528 | 5554 | | YGDAVVYRGTTTYKLNVGDYFVLTSH (SEQ ID NO: 27985) |
| 13 | Nsp13 | 5607 | 5646 | | PGTGKSHFAIGLALYYPSARIVYTACSHAAVDALCEKAL (SEQ |
| | | | | | ID NO: 27986) |
| 14 | Nsp13 | 5699 | 5718 | | ISMATNYDLSVVNARLRAK (SEQ ID NO: 27987) |
| 15 | Nsp13 | 5731 | 5743 | | PRTLLTKGTLEPEYFNSVCRLM (SEQ ID NO: 27988) |
| 16 | Nsp13 | 5763 | 5785 | | TCRRCPAEIVDTVSALVYDNKL (SEQ ID NO: 27989) |
| 17 | Nsp13 | 5797 | 5819 | | MFYKGVITHDVSSAINRPQIGV (SEQ ID NO: 27990) |
| 18 | Nsp13 | 5869 | 5895 | | FTQTTETAHSCNVNRENVAITRAKVG (SEQ ID NO: 27991) |
| 19 | nsp8 | 3992 | 4021 | | RDAAMQRKLEKMADQAMTQMYKQARSEDK (SEQ ID |
| | | | | | NO: 27992) |
| 20 | nsp8 | 4085 | 4108 | | GTTFTYASALWEIQQVVDADSKI (SEQ ID NO: 27993) |
| 21 | nsp8 | 4118 | 4135 | | SPNLAWPLIVTALRANS (SEQ ID NO: 27994) |
| 22 | nsp14 | 5972 | 5993 | | DMTYRRLISMMGFKMNYQVNG (SEQ ID NO: 27995) |
| 23 | nsp14 | 6097 | 6130 | | TLKNLSDRVVFVLWAHGFELTSMKYFVKIGPER (SEQ ID |
| | | | | | NO: 27996) |
| 24 | nsp14 | 6183 | 6207 | | LYCQVHGNAHVASCDAIMTRCLAV (SEQ ID NO: 27997) |
| 25 | nsp14 | 6248 | 6267 | | DKFPVLHDIGNPKAIKCVP (SEQ ID NO: 27998) |
| 26 | nsp14 | 6338 | 6355 | | CDGGSLYVNKHAFHTPA (SEQ ID NO: 27999) |
| 27 | nsp14 | 6432 | 6449 | | LWVYKQFDTYNLWNTFT (SEQ ID NO: 28000) |
| 28 | N | 100 | 117 | | MKDLSPRWYFYYLGTGP (SEQ ID NO: 28001) |
| 29 | N | 300 | 323 | | WPQIAQFAPSASAFFGMSRIGME (SEQ ID NO: 28002) |
| 30 | nsp5 | 3355 | 3379 | | TANPKTPKYKFVRIQPGQTFSVLA (SEQ ID NO: 28003) |
| 31 | nsp5 | 3418 | 3440 | | CVSFCYMHHMELPTGVHAGTDL (SEQ ID NO: 28004) |
| 32 | nsp5 | 3547 | 3567 | | ALLEDEFTPFDVVRQCSGVT (SEQ ID NO: 28005) |
| 33 | nsp3 | 1718 | 1736 | | NKTVGELGDVRETMSYLF (SEQ ID NO: 28006) |
| 34 | nsp3 | 2670 | 2694 | | NNYMLTYNKVENMTPRDLGACIDC (SEQ ID NO: 28007) |
| 35 | nsp9 | 4224 | 4247 | | VKYLYFIKGLNNLNRGMVLGSLA (SEQ ID NO: 28008) |
| 36 | M | 131 | 148 | | PLLESELVIGAVILRGH (SEQ ID NO: 28009) |
| 37 | M | 193 | 210 | | AAYSRYRIGNYKLNTDH (SEQ ID NO: 28010) |
| 38 | nsp7 | 3887 | 3905 | | WAQCVQLHNDILLAKDTT (SEQ ID NO: 28011) |
| 39 | nsp4 | 3168 | 3195 | | GVSFSTFEEAALCTFLLNKEMYLKLRS (SEQ ID NO: 28012) |

TABLE 16B
TCE13 Cassette

| | | Start | End | SEQ | |
|---|---|---|---|---|---|
| Frame | Gene | aa | aa | ID NO | Amino Acid Sequence |
| 1 | nsp13 | 5528 | 5554 | | YGDAVVYRGTTTYKLNVGDYFVLTSH (SEQ ID NO: 27985) |
| 2 | nsp12 | 4829 | 4854 | | KHFFFAQDGNAAISDYDYYRYNLPT (SEQ ID NO: 28013) |
| 3 | N | 300 | 323 | | WPQIAQFAPSASAFFGMSRIGME (SEQ ID NO: 28002) |
| 4 | nsp12 | 4878 | 4908 | | CINANQVIVNNLDKSAGFPFNKWGKARLYY (SEQ ID |
| | | | | | NO: 27979) |
| 5 | nsp12 | 5303 | 5320 | | TSRYWEPEFYEAMYTPH (SEQ ID NO: 27984) |
| 6 | nsp8 | 3992 | 4021 | | RDAAMQRKLEKMADQAMTQMYKQARSEDK (SEQ ID |
| | | | | | NO: 27992) |
| 7 | nsp13 | 5607 | 5646 | | PGTGKSHFAIGLALYYPSARIVYTACSHAAVDALCEKAL (SEQ |
| | | | | | ID NO: 27986) |
| 8 | nsp12 | 5208 | 5235 | | TMLVKQGDDYVYLPYPDPSRILGAGCF (SEQ ID NO: 27982) |
| 9 | nsp8 | 4085 | 4108 | | GTTFTYASALWEIQQVVDADSKI (SEQ ID NO: 27993) |
| 10 | nsp13 | 5869 | 5895 | | FTQTTETAHSCNVNRENVAITRAKVG (SEQ ID NO: 27991) |
| 11 | N | 100 | 117 | | MKDLSPRWYFYYLGTGP (SEQ ID NO: 28001) |
| 12 | nsp12 | 5241 | 5296 | | TDGTLMIERFVSLAIDAYPLTKHPNQEYADVFHLYLQYIRKLH |
| | | | | | DELTGHMLDMYS (SEQ ID NO: 27983) |
| 13 | nsp12 | 4705 | 4745 | | NVLFSTVFPPTSFGPLVRKIFVDGVPFVVSTGYHFRELGV (SEQ |
| | | | | | ID NO: 27976) |
| 14 | M | 131 | 148 | | PLLESELVIGAVILRGH (SEQ ID NO: 28009) |
| 15 | nsp3 | 2670 | 2694 | | NNYMLTYNKVENMTPRDLGACIDC (SEQ ID NO: 28007) |
| 16 | nsp14 | 6102 | 6130 | | SDRVVFVLWAHGFELTSMKYFVKIGPER (SEQ ID NO: 28014) |
| 17 | nsp12 | 5132 | 5154 | | FVNEFYAYLRKHFSMMILSDDA (SEQ ID NO: 27981) |
| 18 | nsp13 | 5763 | 5785 | | TCRRCPAEIVDTVSALVYDNKL (SEQ ID NO: 27989) |
| 19 | nsp8 | 4118 | 4135 | | SPNLAWPLIVTALRANS (SEQ ID NO: 27994) |
| 20 | nsp3 | 1718 | 1736 | | NKTVGELGDVRETMSYLF (SEQ ID NO: 28006) |
| 21 | nsp14 | 6432 | 6449 | | LWVYKQFDTYNLWNTFT (SEQ ID NO: 28000) |
| 22 | nsp5 | 3547 | 3567 | | ALLEDEFTPFDVVRQCSGVT (SEQ ID NO: 28005) |
| 23 | nsp12 | 4948 | 4994 | | VAGVSICSTMTNRQFHQKLLKSIAATRGATVVIGTSKFYGGW |
| | | | | | HNML (SEQ ID NO: 27980) |
| 24 | M | 193 | 210 | | AAYSRYRIGNYKLNTDH (SEQ ID NO: 28010) |
| 25 | nsp12 | 4751 | 4785 | | NLHSSRLSFKELLVYAADPAMHAASGNLLLDKRT (SEQ ID |
| | | | | | NO: 27977) |
| 26 | nsp14 | 6183 | 6207 | | LYCQVHGNAHVASCDAIMTRCLAV (SEQ ID NO: 27997) |
| 27 | nsp14 | 5972 | 5993 | | DMTYRRLISMMGFKMNYQVNG (SEQ ID NO: 27995) |
| 28 | nsp14 | 6248 | 6267 | | DKFPVLHDIGNPKAIKCVP (SEQ ID NO: 27998) |
| 29 | nsp5 | 3355 | 3379 | | TANPKTPKYKFVRIQPGQTFSVLA (SEQ ID NO: 28003) |
| 30 | nsp12 | 4414 | 4431 | | GTGTSTDVVYRAFDIYN (SEQ ID NO: 27974) |
| 31 | nsp14 | 6338 | 6355 | | CDGGSLYVNKHAFHTPA (SEQ ID NO: 27999) |

TABLE 16C
TCE14 Cassette

| | | Start | End | SEQ | |
|---|---|---|---|---|---|
| Frame | Gene | aa | aa | ID NO | Amino Acid Sequence |
| 1 | nsp13 | 5528 | 5554 | | YGDAVVYRGTTTYKLNVGDYFVLTSH (SEQ ID NO: 27985) |
| 2 | N | 100 | 117 | | MKDLSPRWYFYYLGTGP (SEQ ID NO: 28001) |
| 3 | nsp12 | 5241 | 5296 | | TDGTLMIERFVSLAIDAYPLTKHPNQEYADVFHLYLQYIRKLHD |
| | | | | | ELTGHMLDMYS (SEQ ID NO: 27983) |
| 4 | nsp12 | 5136 | 5153 | | FYAYLRKHFSMMILSDD (SEQ ID NO: 28015) |
| 5 | nsp14 | 6432 | 6449 | | LWVYKQFDTYNLWNTFT (SEQ ID NO: 28000) |
| 6 | nsp8 | 3996 | 4021 | | MQRKLEKMADQAMTQMYKQARSEDK (SEQ ID NO: 28016) |
| 7 | nsp13 | 5607 | 5636 | | PGTGKSHFAIGLALYYPSARIVYTACSHA (SEQ ID NO: 28017) |
| 8 | nsp12 | 5303 | 5320 | | TSRYWEPEFYEAMYTPH (SEQ ID NO: 27984) |
| 9 | nsp12 | 4878 | 4908 | | CINANQVIVNNLDKSAGFPFNKWGKARLYY (SEQ ID NO: 27979) |
| 10 | nsp5 | 3547 | 3567 | | ALLEDEFTPFDVVRQCSGVT (SEQ ID NO: 28005) |
| 11 | nsp12 | 4948 | 4994 | | VAGVSICSTMTNRQFHQKLLKSIAATRGATVVIGTSKFYGGWH |
| | | | | | NML (SEQ ID NO: 27980) |
| 12 | nsp12 | 5208 | 5235 | | TMLVKQGDDYVYLPYPDPSRILGAGCF (SEQ ID NO: 27982) |
| 13 | M | 193 | 210 | | AAYSRYRIGNYKLNTDH (SEQ ID NO: 28010) |
| 14 | nsp12 | 4751 | 4785 | | NLHSSRLSFKELLVYAADPAMHAASGNLLLDKRT (SEQ ID |
| | | | | | NO: 27977) |
| 15 | nsp12 | 4721 | 4745 | | VRKIFVDGVPFVVSTGYHFRELGV (SEQ ID NO: 28018) |
| 16 | M | 131 | 148 | | PLLESELVIGAVILRGH (SEQ ID NO: 28009) |
| 17 | nsp3 | 2670 | 2694 | | NNYMLTYNKVENMTPRDLGACIDC (SEQ ID NO: 28007) |
| 18 | nsp14 | 6102 | 6130 | | SDRVVFVLWAHGFELTSMKYFVKIGPER (SEQ ID NO: 28014) |
| 19 | nsp12 | 4829 | 4854 | | KHFFFAQDGNAAISDYDYYRYNLPT (SEQ ID NO: 28013) |
| 20 | N | 300 | 323 | | WPQIAQFAPSASAFFGMSRIGME (SEQ ID NO: 28002) |
| 21 | nsp13 | 5763 | 5785 | | TCRRCPAEIVDTVSALVYDNKL (SEQ ID NO: 27989) |
| 22 | nsp5 | 3355 | 3379 | | TANPKTPKYKFVRIQPGQTFSVLA (SEQ ID NO: 28003) |
| 23 | nsp12 | 4414 | 4431 | | GTGTSTDVVYRAFDIYN (SEQ ID NO: 27974) |
| 24 | nsp14 | 6338 | 6355 | | CDGGSLYVNKHAFHTPA (SEQ ID NO: 27999) |

TABLE 16D
TCE15 Cassette

| | | | | SEQ | |
|---|---|---|---|---|---|
| | | Start | End | ID | |
| Frame | Gene | aa | aa | NO | Amino Acid Sequence |
| 1 | N | 300 | 323 | | WPQIAQFAPSASAFFGMSRIGME (SEQ ID NO: 28002) |
| 2 | nsp12 | 5256 | 5296 | | DAYPLTKHPNQEYADVFHLYLQYIRKLHDELTGHMLDMYS (SEQ |
| | | | | | ID NO: 28019) |
| 3 | nsp12 | 5136 | 5153 | | FYAYLRKHFSMMILSDD (SEQ ID NO: 28015) |
| 4 | nsp13 | 5607 | 5636 | | PGTGKSHFAIGLALYYPSARIVYTACSHA (SEQ ID NO: 28017) |
| 5 | nsp12 | 5303 | 5320 | | TSRYWEPEFYEAMYTPH (SEQ ID NO: 27984) |
| 6 | nsp14 | 6338 | 6355 | | CDGGSLYVNKHAFHTPA (SEQ ID NO: 27999) |
| 7 | nsp12 | 4887 | 4905 | | NNLDKSAGFPFNKWGKAR (SEQ ID NO: 27963) |
| 8 | nsp3 | 2675 | 2692 | | TYNKVENMTPRDLGACI (SEQ ID NO: 27965) |
| 9 | nsp12 | 4948 | 4994 | | VAGVSICSTMTNRQFHQKLLKSIAATRGATVVIGTSKFYGGWH |
| | | | | | NML (SEQ ID NO: 27980) |
| 10 | nsp12 | 5208 | 5235 | | TMLVKQGDDYVYLPYPDPSRILGAGCF (SEQ ID NO: 27982) |
| 11 | M | 193 | 210 | | AAYSRYRIGNYKLNTDH (SEQ ID NO: 28010) |
| 12 | nsp12 | 4751 | 4785 | | NLHSSRLSFKELLVYAADPAMHAASGNLLLDKRT (SEQ ID |
| | | | | | NO: 27977) |
| 13 | nsp12 | 4721 | 4745 | | VRKIFVDGVPFVVSTGYHFRELGV (SEQ ID NO: 28018) |
| 14 | M | 131 | 148 | | PLLESELVIGAVILRGH (SEQ ID NO: 28009) |
| 15 | nsp12 | 4829 | 4854 | | KHFFFAQDGNAAISDYDYYRYNLPT (SEQ ID NO: 28013) |
| 16 | N | 100 | 117 | | MKDLSPRWYFYYLGTGP (SEQ ID NO: 28001) |

### Pancorona Sequences

Certain Pancorona vaccine sequences evaluated herein are presented below in Table 17.

TABLE 17
Pancorona Sequences

Spike-Delta ΔF-2P amino acid sequence: 1271 aa furin mutation in bold and double proline
substitution in bold and italics
MFVFLVLLPLVSSQCVNLRTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLDVYYHKNNKSWMESGVYSSANNCTFEYVSQPFLMDL
EGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSE
TKCTLKSFTVEKGIYQTSNFRVQPTESIVRFPNITNLCPFGEVENATRFASVYAWNRKRI
SNCVADYSVLYNSASFSTFKCYGVSPTKLNDLCFTNVYADSFVIRGDEVRQIAPGQTGK
IADYNYKLPDDFTGCVIAWNSNNLDSKVGGNYNYRYRLFRKSNLKPFERDISTEIYQA
GSKPCNGVEGENCYFPLQSYGFQPTNGVGYQPYRVVVLSFELLHAPATVCGPKKSTNL
VKNKCVNFNFNGLTGTGVLTESNKKFLPFQQFGRDIADTTDAVRDPQTLEILDITPCSF
GGVSVITPGTNTSNQVAVLYQGVNCTEVPVAIHADQLTPTWRVYSTGSNVFQTRAGCL
IGAEHVNNSYECDIPIGAGICASYQTQTNSRGSASSVASQSIIAYTMSLGAENSVAYSNN
SIAIPTNFTISVTTEILPVSMTKTSVDCTMYICGDSTECSNLLLQYGSFCTQLNRALTGIA
VEQDKNTQEVFAQVKQIYKTPPIKDFGGFNFSQILPDPSKPSKRSFIEDLLENKVTLADA
GFIKQYGDCLGDIAARDLICAQKFNGLTVLPPLLTDEMIAQYTSALLAGTITSGWTFGA
GAALQIPFAMQMAYRENGIGVTQNVLYENQKLIANQFNSAIGKIQDSLSSTASALGKLQ
NVVNQNAQALNTLVKQLSSNFGAISSVLNDILSRLDPPEAEVQIDRLITGRLQSLQTYV
TQQLIRAAEIRASANLAATKMSECVLGQSKRVDFCGKGYHLMSFPQSAPHGVVFLHVT
YVPAQEKNFTTAPAICHDGKAHFPREGVFVSNGTHWFVTQRNFYEPQIITTDNTFVSGN
CDVVIGIVNNTVYDPLQPELDSFKEELDKYFKNHTSPDVDLGDISGINASVVNIQKEIDR
LNEVAKNLNESLIDLQELGKYEQYIKWPWYIWLGFIAGLIAIVMVTIMLCCMTSCCSCL
KGCCSCGSCCKFDEDDSEPVLKGVKLHYT (SEQ ID NO: 28020)
KP886809 RBD: 204 aa with SARS-COV2 leader sequence in bold italic
MFVFLVLLPLVSSRVAPVTEVVRFPNITNLCPFDKVFNATRFPSVYAWERTKISDCVAD
YTVFYNSTSFSTENCYGVSPSKLIDLCFTSVYADTFLIRFSEVRQVAPGQTGVIADYNYK
LPDDFTGCVIAWNTAKYDVGSYFYRSHRSSKLKPFERDLSSEENGARTLSTYDFNQNV
PLEYQATRVVVLSFELLNAPATVCGPKLSTSLVKNQCVNF(SEQ ID NO: 28021)
KJ473815 RBD: 205 aa with SARS-COV2 leader sequence in bold italic
MFVFLVLLPLVSSRVSPTQEVVRFPNITNRCPFDKVFNATRFPNVYAWERTKISDCVAD
YTVLYNSTSFSTFKCYGVSPSKLIDLCFTSVYADTFLIRSSEVRQVAPGETGVIADYNYK
LPDDFTGCVIAWNTAKQDTGNYYYRSHRKTKLKPFERDLSSDDGNGVYTLSTYDFNP
NVPVAYQATRVVVLSFELLNAPATVCGPKLSTQLVKNQCVNF (SEQ ID NO: 28022)
MK211376 RBD 222 aa with SARS-COV2 leader sequence in bold italic
MFVFLVLLPLVSSRVAPSKEVVRFPNITNLCPFGEVENATTFPSVYAWERKRISNCVAD
YSVLYNSTSFSTFKCYGVSATKLNDLCFSNVYADSFVVKGDDVRQIAPGQTGVIADYN
YKLPDDFMGCVLAWNTRNIDATSTGNYNYKYRSLRHGKLRPFERDISNVPFSPDGKPC
TPPAFNCYWPLNDYGFFTTNGIGYQPYRVVVLSFELLNAPATVCGPKLSTDLIKNQCVN
F (SEQ ID NO: 28023)
DQ648856 RBD 204 aa with SARS-COV2 leader sequence in bold italic
MFVFLVLLPLVSSRVSPVTEVVRFPNITNLCPFDKVFNATRFPSVYAWERTKISDCVAD
YTVFYNSTSFSTENCYGVSPSKLIDLCFTSVYADTFLIRFSEVRQVAPGQTGVIADYNYK
LPDDFTGCVIAWNTAKQDVGSYFYRSHRSSKLKPFERDLSSVEENGRTLSTYDFNQNV
PLEYQATRVVVLSFELLNAPATVCGPKLSTSLVKNQCVNF (SEQ ID NO: 28024)
GQ153542 RBD 205 aa with SARS-COV2 leader sequence in bold italic
MFVFLVLLPLVSSRVSPTQEVIRFPNITNRCPFDRVFNASRFPSVYAWERTKISECVADY
TVLYNSTSFSTFKCYGVSPSKLIDLCFTSVYADTFLIRSSEVRQVAPGETGVIADYNYKL
PDDFTGCVIAWNTAKQDTGNYYYRSHRKTKLKPFERDLSSDDGNGVYTLSTYDFNPN
VPVAYQATRVVVLSFELLNAPATVCGPKLSTQLVKNQCVNF (SEQ ID NO: 28025)
JX993988.1RBD 204 aa with SARS-COV2 leader sequence in bold italic
MFVFLVLLPLVSSRVSPSTEVIRFPNITNRCPFDRVENASRFPSVYAWERTKISDCVADY
TVLYNSTSFSTFKCYGVSPSKLIDLCFTSVYADTFLIRFSEVRQIAPGETGVIADYNYKLP
DEFTGCVIAWNTANQDRGQYYYRSSRKTKLKPFERDLSSDENGVRTLSTYDFYPSVPL
EYQATRVVVLSFELLNAPATVCGPKLSTSLIKNQCVNF (SEQ ID NO: 28026)
NC_004718 RBD 204 aa with SARS-COV2 leader sequence in bold italic
MFVFLVLLPLVSSRVVPSGDVVRFPNITNLCPFGEVFNATKFPSVYAWERKKISNCVAD
YSVLYNSTFFSTFKCYGVSATKLNDLCFSNVYADSFVVKGDDVRQIAPGQTGVIADYN
YKLPDDFMGCVLAWNTRNIDATSTGNYNYKYRYLRHGKLRPFERDISNVPFSPDGKPC
TPPALNCYWPLNDYGFYTTTGIGYQPYRVVVLSFELLNAPATVCGPKLSTDLIKNQCV
NF (SEQ ID NO: 28027)
T4 Fibritin Trimerization Domain
GYIPEAPRDGQAYVRKDGEWVFLSTFLSPA (SEQ ID NO: 28028)
MTQ Trimerization Domain
GGSGGIKEEIAKIKEEQAKIKEKIAEIEKRIAEIEKRIAGGCC (SEQ ID NO: 28029)
GCN4 Trimerization Domain
RMKQIEDKIEEILSKIYHIENEIARIKKLIGER (SEQ ID NO: 28030)
Group 2 3X RBD Protein Sequence Example with Trimerization domains, linkers, T2A
and Furin sites 859 aa
MFVFLVLLPLVSSRVSPVTEVVRFPNITNLCPFDKVFNATRFPSVYAWERTKISDCVAD
YTVFYNSTSFSTENCYGVSPSKLIDLCFTSVYADTFLIRFSEVRQVAPGQTGVIADYNYK
LPDDFTGCVIAWNTAKQDVGSYFYRSHRSSKLKPFERDLSSVEENGRTLSTYDFNQNV
PLEYQATRVVVLSFELLNAPATVCGPKLSTSLVKNQCVNFGGSGGSGGYIPEAPRDGQ
AYVRKDGEWVFLSTFLSPAGGSGGVRAEGRGSLLTCGDVEENPGPRRRARMFVFLVL
LPLVSSRVSPTQEVVRFPNITNRCPFDKVFNATRFPNVYAWERTKISDCVADYTVLYNS
TSFSTFKCYGVSPSKLIDLCFTSVYADTFLIRSSEVRQVAPGETGVIADYNYKLPDDFTG
CVIAWNTAKQDTGNYYYRSHRKTKLKPFERDLSSDDGNGVYTLSTYDFNPNVPVAYQ
ATRVVVLSFELLNAPATVCGPKLSTQLVKNQCVNFGGSGGSGGGSGGIKEEIAKIKEEQ
AKIKEKIAEIEKRIAEIEKRIAGGCCGGSGGVRAEGRGSLLTCGDVEENPGPRRRARMFV
FLVLLPLVSSRVAPSKEVVRFPNITNLCPFGEVENATTFPSVYAWERKRISNCVADYSVL
YNSTSFSTFKCYGVSATKLNDLCFSNVYADSFVVKGDDVRQIAPGQTGVIADYNYKLP
DDFMGCVLAWNTRNIDATSTGNYNYKYRSLRHGKLRPFERDISNVPFSPDGKPCTPPA
FNCYWPLNDYGFFTTNGIGYQPYRVVVLSFELLNAPATVCGPKLSTDLIKNQCVNFGG
SGGSGRMKQIEDKIEEILSKIYHIENEIARIKKLIGER (SEQ ID NO: 28031)
Codon optimized SARS-COV-2 Delta Sequence, Kozak sequence in bold italic
GCCACCATGTTTGTCTTCCTGGTCTTGCTGCCGCTGGTGAGCAGCCAGTGCGTGAAT
CTCcgCACCCGCACCCAGCTTCCACCTGCCTACACTAACAGCTTCACCCGAGGGGTG
TATTACCCTGACAAGGTATTCCGGTCCTCCGTCCTCCATAGCACGCAGGACCTTTTT
CTGCCCTTCTTCTCAAATGTGACATGGTTCCATGCCATTCACGTGAGCGGCACGAAT
GGAACGAAGCGCTTTGATAACCCCGTGCTGCCTTTCAATGACGGCGTCTACTTCGC
CTCCACTGAAAAGTCAAACATCATCCGGGGCTGGATCTTTGGCACCACTCTTGATT
CAAAGACCCAGTCACTGCTGATTGTGAACAATGCTACAAACGTGGTTATCAAGGTG
TGTGAGTTTCAGTTCTGTAACGATCCATTTTTGGatGTGTACTACCACAAGAACAAC
AAGTCCTGGATGgAGTCTgGAGTGTATAGCTCTGCTAACAACTGCACCTTCGAGTAC
GTGTCCCAGCCTTTCCTTATGGACCTGGAAGGCAAACAGGGCAATTTCAAAAACCT
GAGAGAGTTCGTGTTTAAGAACATTGACGGATACTTCAAAATTTATTCTAAGCACA
CACCAATTAACTTAGTGCGGGACCTACCCCAAGGCTTTAGCGCCCTAGAGCCCCTG
GTTGACCTGCCCATTGGGATCAATATAACAAGGTTCCAAACTCTACTGGCTCTGCA
TAGAAGTTATCTGACCCCAGGAGACAGCTCTAGTGGTTGGACCGCCGGCGCAGCAG
CCTACTATGTCGGGTACTTACAGCCACGCACGTTCCTTCTGAAGTACAATGAGAAC
GGGACAATCACTGACGCAGTAGACTGTGCACTGGACCCGCTAAGCGAGACTAAGT
GCACACTTAAATCCTTCACGGTGGAGAAAGGCATTTATCAGACCTCTAACTTCAGG
GTGCAGCCAACAGAAAGCATTGTGCGATTCCCAAATATTACTAATCTTTGCCCTTTC
GGGGAGGTCTTTAATGCAACTAGATTCGCATCAGTCTATGCGTGGAACCGCAAACG
CATTTCCAATTGTGTCGCAGACTACTCAGTGCTGTACAACTCTGCCTCTTTCAGTAC
GTTCAAGTGTTACGGAGTGTCACCCACTAAACTGAACGACCTGTGCTTTACAAATG
TCTACGCTGACTCCTTCGTGATTAGGGGAGACGAGGTGAGACAAATTGCCCCCGGA
CAGACTGGGAAGATTGCCGACTACAATTATAAGCTTCCTGATGATTTCACTGGCTG
TGTTATTGCCTGGAATAGTAACAATCTGGATAGCAAGGTGGGAGGCAACTATAACT
ACagaTATCGACTGTTTAGGAAGAGTAATCTGAAACCATTTGAGCGGGATATTTCCA
CAGAAATTTACCAGGCCGGGAGCAaACCATGTAATGGGGTGGAGGGATTTAATTGT
TACTTCCCACTCCAGAGCTATGGTTTCCAACCCACCAATGGAGTGGGTTACCAGCC
CTATAGAGTCGTGGTGCTTAGTTTTGAGCTGCTTCACGCCCCAGCAACCGTCTGCGG
TCCCAAAAAGTCGACCAATCTCGTGAAAAACAAATGCGTAAACTTCAACTTTAACG
GCTTAACAGGAACCGGCGTGCTCACCGAAAGCAACAAGAAATTCCTTCCATTTCAG
CAATTCGGAAGGGACATCGCCGACACAACAGACGCGGTGAGGGACCCACAGACTC
TGGAGATACTGGACATCACTCCTTGTTCGTTTGGGGGCGTCTCGGTCATCACACCCG
GGACTAATACTAGTAATCAGGTAGCAGTTTTATATCAAGGCGTCAACTGTACCGAA
GTACCTGTGGCCATACACGCTGATCAGCTAACGCCAACATGGCGAGTCTATTCCAC
CGGCTCTAACGTTTTTCAGACCAGGGCTGGGTGCCTGATAGGGGCAGAGCACGTCA
ATAATTCCTATGAGTGTGATATCCCCATAGGTGCGGGGATCTGTGCCAGCTATCAA
ACCCAAACCAATTCACgAgGGaGcGCAaGcTCTGTGGCTTCTCAGAGCATAATTGCAT
ATACAATGTCACTGGGCGCTGAGAATAGCGTTGCATACTCTAATAACAGCATAGCC
ATTCCCACGAACTTTACTATCAGTGTGACAACCGAAATATTGCCAGTTTCGATGAC
CAAAACTAGCGTGGATTGCACGATGTACATCTGTGGAGACTCTACCGAATGCAGCA
ATCTGCTATTACAATATGGCAGCTTCTGTACACAGTTAAATCGAGCCTTGACAGGC
ATCGCAGTGGAACAGGACAAAAATACTCAAGAGGTGTTTGCACAGGTGAAGCAAA
TCTACAAAACGCCCCCCATTAAAGATTTTGGCGGGTTCAATTTTTCACAAATTCTCC
CCGACCCGTCTAAGCCGAGTAAGCGGTCCTTCATCGAAGATCTGCTCTTTAACAAA
GTAACCCTCGCCGATGCCGGCTTTATTAAGCAGTATGGCGACTGCCTGGGGGATAT
AGCCGCTCGTGACCTGATTTGCGCCCAGAAGTTCAATGGTCTGACCGTGTTGCCTCC
TTTATTGACCGATGAAATGATTGCCCAGTACACTAGTGCCCTGCTGGCCGGCACTA
TCACGTCTGGGTGGACCTTCGGAGCTGGTGCCGCCTTGCAGATACCTTTTGCAATGC
AGATGGCCTATAGGTTTAATGGTATCGGAGTGACTCAGAACGTACTGTACGAGAAC
CAGAAGCTCATCGCTAATCAATTTAACTCCGCTATCGGAAAAATCCAGGACAGCCT
CTCTTCTACAGCTAGCGCTCTGGGCAAACTGCAGaATGTCGTTAATCAGAATGCCCA
GGCCCTGAACACCTTGGTTAAACAACTATCTTCCAACTTCGGGGCCATATCCAGTG
TGTTGAATGATATTCTCTCCCGCTTGGATccacctGAAGCTGAGGTGCAGATCGATCGC
TTGATCACCGGCAGACTGCAGTCCCTCCAGACATATGTAACTCAGCAGCTGATTAG
AGCCGCCGAGATAAGGGCAAGTGCGAATCTGGCTGCCACCAAGATGAGCGAATGT
GTATTGGGCCAGAGCAAACGAGTTGATTTTTGCGGTAAGGGGTATCATTTAATGTC
TTTCCCTCAATCCGCACCTCATGGCGTAGTTTTCCTGCATGTGACTTATGTCCCGGC
TCAGGAGAAGAATTTTACCACAGCCCCCGCGATCTGCCATGACGGAAAGGCCCACT
TCCCCCGGGAAGGCGTGTTTGTATCCAATGGGACTCACTGGTTTGTCACTCAGCGA
AATTTTTATGAACCACAGATCATCACCACTGACAACACATTTGTTAGTGGAAACTG
CGATGTGGTCATCGGCATCGTGAATAACACTGTCTATGATCCACTGCAACCTGAAC
TGGATTCTTTTAAAGAGGAACTCGACAAGTATTTTAAAAACCACACTAGCCCTGAC
GTGGATCTCGGTGACATTTCTGGCATCAACGCTAGCGTAGTGAACATTCAGAAAGA
GATAGATAGACTTAATGAGGTGGCCAAGAACCTCAACGAAAGTCTGATCGACCTCC
AGGAACTGGGGAAATACGAGCAGTACATTAAATGGCCTTGGTACATATGGCTGGG
GTTCATTGCTGGGCTGATCGCAATAGTGATGGTGACCATAATGCTCTGTTGCATGA
CTAGCTGCTGCAGCTGCCTGAAGGGCTGCTGTAGTTGTGGGTCATGTTGTAAGTTTG
ACGAAGATGATAGCGAGCCTGTCCTTAAAGGAGTGAAGCTCCACTACACCTAGTAA
(SEQ ID NO: 28032)
Group 2 3X RBD Sequence Codon Optimized Sequence; Kozak sequence in bold italic
GCCACCATGTTTGTCTTCCTGGTCTTGCTGCCGCTGGTGAGCAGCCGCGTGTCTCCC
GTTACTGAAGTCGTCCGGTTCCCAAACATCACAAATCTCTGTCCTTTTGACAAAGTG
TTTAATGCTACGCGCTTTCCTTCTGTATACGCCTGGGAGCGGACCAAGATCTCAGAT
TGCGTGGCCGACTACACCGTGTTCTATAATTCCACCTCCTTCAGCACCTTCAACTGC
TACGGCGTGAGCCCATCTAAACTGATTGATTTGTGCTTTACGAGTGTGTACGCGGA
TACTTTCCTGATTAGGTTTTCGGAAGTTCGTCAAGTAGCCCCCGGCCAGACGGGAG
TGATAGCTGATTACAACTATAAACTGCCTGATGACTTCACCGGCTGTGTAATAGCG
TGGAATACGGCTAAGCAGGATGTAGGCTCCTACTTCTATCGTTCGCACCGGTCGTC
GAAATTAAAGCCGTTTGAGCGTGACCTCTCGTCAGTAGAGGAAAACGGTAGAACG
CTGAGCACCTATGACTTTAATCAAAACGTGCCCTTAGAGTATCAGGCCACTAGAGT
AGTAGTCCTCTCATTTGAACTGTTAAATGCACCCGCAACAGTTTGTGGGCCGAAGT
TGAGCACTAGCCTAGTTAAGAACCAGTGTGTGAACTTCGGAGGTAGCGGCGGTTCT
GGAGGCTACATTCCAGAAGCTCCACGCGATGGCCAGGCTTATGTGCGGAAAGATG
GCGAGTGGGTGTTTCTCAGCACCTTTCTGAGTCCAGCTGGCGGTAGCGGCGGGGTT
CGGGCAGAGGGCAGAGGCTCACTGCTCACGTGTGGTGATGTAGAAGAGAATCCAG
GTCCTAGGAGGCGAGCACGGATGTTCGTATTTCTGGTGCTGCTGCCCCTAGTGAGC
TCCAGGGTGTCGCCCACCCAGGAGGTAGTGAGGTTCCCTAACATAACTAATCGGTG
CCCCTTTGATAAAGTTTTTAACGCCACTAGATTCCCAAATGTGTATGCATGGGAGA
GAACTAAAATTTCAGACTGCGTCGCTGACTACACCGTGTTGTATAATAGTACCAGT
TTCAGTACATTCAAGTGTTACGGCGTGTCCCCTTCCAAACTCATCGACCTTTGCTTC
ACCAGCGTCTACGCGGATACTTTTCTTATTAGATCTTCGGAGGTCCGCCAGGTAGC
ACCAGGGGAAACAGGAGTAATAGCTGATTATAACTACAAACTACCAGACGATTTC
ACAGGTTGCGTGATCGCCTGGAACACAGCTAAGCAGGACACGGGAAATTACTACT
ATAGGAGCCATAGGAAGACCAAGCTCAAGCCTTTTGAGAGAGATCTGTCTTCTGAC
GACGGAAATGGCGTTTATACACTGTCAACCTACGACTTCAATCCTAATGTGCCCGT
TGCCTACCAGGCCACCAGAGTCGTGGTGCTGAGCTTCGAGCTGCTGAACGCCCCTG
CTACAGTCTGCGGCCCTAAGCTGAGCACACAGCTGGTGAAGAATCAGTGTGTGAAT
TTTGGAGGTAGCGGCGGTTCTGGAGGGGGGTCTGGAGGCATAAAGGAAGAGATTG
CCAAGATTAAGGAAGAACAGGCAAAGATAAAAGAAAAGATTGCAGAGATTGAAA
AAAGGATTGCTGAGATCGAGAAAAGGATAGCAGGAGGTTGTTGCGGCGGTAGCGG
CGGTGTCCGTGCTGAAGGACGCGGGTCCCTTCTCACCTGCGGCGACGTTGAGGAAA
ACCCTGGCCCTAGAAGGCGAGCACGGATGTTCGTGTTCCTGGTGCTTCTCCCTCTGG
TGTCTAGCCGTGTGGCCCCCAGCAAGGAAGTCGTGCGCTTCCCCAACATCACAAAT
CTCTGTCCTTTTGGAGAGGTCTTCAATGCAACCACATTCCCTTCCGTGTACGCCTGG
GAGAGAAAACGGATTTCCAACTGCGTGGCTGACTACAGCGTGCTTTATAATTCTAC
CAGTTTCTCTACCTTCAAATGCTACGGCGTGTCTGCCACAAAACTAAATGATCTGTG
CTTCAGCAATGTGTATGCCGACAGCTTTGTTGTGAAGGGAGATGATGTGCGGCAGA
TTGCACCCGGCCAGACCGGGGTTATCGCCGACTACAACTATAAGCTGCCCGATGAC
TTCATGGGCTGCGTGCTCGCTTGGAACACAAGAAACATTGATGCCACCTCAACCGG
AAACTACAATTACAAATATCGAAGCTTGCGGCACGGGAAACTGAGGCCATTTGAA
AGGGACATCAGCAACGTGCCCTTCTCACCGGATGGAAAGCCATGCACCCCCCCTGC
CTTTAATTGCTACTGGCCCCTGAACGACTATGGCTTCTTCACAACCAATGGCATTGG
ATATCAGCCCTACAGAGTGGTGGTGCTGAGTTTTGAGCTGCTGAACGCTCCCGCTA
CAGTGTGCGGACCCAAGCTCAGCACAGACCTGATCAAGAACCAGTGTGTGAACTTC
GGAGGTAGCGGCGGTTCTGGAAGAATGAAACAGATTGAGGATAAAATTGAAGAGA
TATTGTCAAAGATCTATCACATAGAGAACGAAATCGCCCGAATAAAGAAGCTGATT
GGGGAACGCTAGTGA (SEQ ID NO: 28033)
Group 1 3X RBD Sequence Codon Optimized Sequence; Kozak sequence in bold italic
GCCACCATGTTTGTCTTCCTGGTCTTGCTGCCGCTGGTGAGCAGCAGGGTAGCGCCC
GTGACTGAGGTAGTCCGCTTCCCCAACATCACCAACCTGTGCCCATTTGATAAAGT
GTTCAATGCCACAAGGTTCCCATCTGTATACGCATGGGAACGGACCAAAATTAGTG
ACTGTGTGGCCGATTACACCGTGTTTTACAACAGTACCTCTTTTTCCACATTCAACT
GCTATGGAGTTTCTCCTTCAAAGCTTATCGATCTATGTTTCACTTCTGTTTACGCTGA
CACTTTTCTGATCCGTTTCTCAGAAGTCCGACAGGTCGCCCCTGGTCAAACAGGGG
TGATAGCGGATTATAATTACAAGCTTCCGGATGACTTCACTGGCTGCGTTATTGCTT
GGAACACTGCCAAATATGACGTCGGCTCATACTTCTATCGCTCACACCGGTCGAGC
AAGCTCAAGCCTTTTGAAAGGGATCTCAGCAGCGAGGAGAATGGGGCCAGAACCC
TGAGCACCTATGACTTTAACCAGAATGTGCCACTGGAATACCAGGCTACGAGAGTG
GTGGTCCTGAGCTTCGAGCTGTTGAATGCACCCGCAACAGTTTGTGGCCCTAAACT
GTCCACAAGCCTGGTGAAGAATCAGTGTGTAAACTTTGGAGGTAGCGGCGGTTCTG
GAGGCTACATTCCAGAAGCTCCACGCGATGGCCAGGCTTATGTGCGGAAAGATGG
CGAGTGGGTGTTTCTCAGCACCTTTCTGAGTCCAGCTGGCGGTAGCGGCGGGGTTC
GGGCAGAGGGCAGAGGCTCACTGCTCACGTGTGGTGATGTAGAAGAGAATCCAGG
TCCTAGGAGGCGAGCACGGATGTTCGTATTTCTGGTGCTGCTGCCCCTAGTGAGCT
CCAGGGTGTCGCCCACCCAGGAGGTAGTGAGGTTCCCTAACATAACTAACCGGTGC
CCCTTTGATAAAGTTTTTAACGCCACTAGATTCCCAAATGTGTATGCATGGGAGAG
AACTAAAATTTCAGACTGCGTCGCTGACTACACCGTGTTGTATAATAGTACCAGTTT
CAGTACATTCAAGTGTTACGGCGTGTCCCCTTCCAAACTCATCGACCTTTGCTTCAC
CAGCGTCTACGCGGATACTTTTCTTATTAGATCTTCGGAGGTCCGCCAGGTAGCACC
AGGGGAAACAGGAGTAATAGCTGATTATAACTACAAACTACCAGACGATTTCACA
GGTTGCGTGATCGCCTGGAACACAGCTAAGCAGGACACGGGAAATTACTACTATA
GGAGCCATAGGAAGACCAAGCTCAAGCCTTTTGAGAGAGATCTGTCTTCTGACGAC
GGAAATGGCGTTTATACACTGTCAACCTACGACTTCAATCCTAATGTGCCCGTTGCC
TACCAGGCCACCAGAGTCGTGGTGCTGAGCTTCGAGCTGCTGAACGCCCCTGCTAC
AGTCTGCGGCCCTAAGCTGAGCACACAGCTGGTGAAGAATCAGTGTGTGAATTTTG
GAGGTAGCGGCGGTTCTGGAGGGGGGTCTGGAGGCATAAAGGAAGAGATTGCCAA
GATTAAGGAAGAACAGGCAAAGATAAAAGAAAAGATTGCAGAGATTGAAAAAAG
GATTGCTGAGATCGAGAAAAGGATAGCAGGAGGTTGTTGCGGCGGTAGCGGCGGT
GTCCGTGCTGAAGGACGCGGGTCCCTTCTCACCTGCGGCGACGTTGAGGAAAACCC
TGGCCCTAGAAGGCGAGCACGGATGTTCGTGTTCCTGGTGCTTCTCCCTCTGGTGTC
TAGCCGTGTGGCCCCCAGCAAGGAAGTCGTGCGCTTCCCCAACATCACAAATCTCT
GTCCTTTTGGAGAGGTCTTCAATGCAACCACATTCCCTTCCGTGTACGCCTGGGAGA
GAAAACGGATTTCCAACTGCGTGGCTGACTACAGCGTGCTTTATAATTCTACCAGT
TTCTCTACCTTCAAATGCTACGGCGTGTCTGCCACAAAACTAAATGATCTGTGCTTC
AGCAATGTGTATGCCGACAGCTTTGTTGTGAAGGGAGATGATGTGCGGCAGATTGC
ACCCGGCCAGACCGGGGTTATCGCCGACTACAACTATAAGCTGCCCGATGACTTCA
TGGGCTGCGTGCTCGCTTGGAACACAAGAAACATTGATGCCACCTCAACCGGAAAC
TACAATTACAAATATCGAAGCTTGCGGCACGGGAAACTGAGGCCATTTGAAAGGG
ACATCAGCAACGTGCCCTTCTCACCGGATGGAAAGCCATGCACCCCCCCTGCCTTT
AATTGCTACTGGCCCCTGAACGACTATGGCTTCTTCACAACCAATGGCATTGGATA
TCAGCCCTACAGAGTGGTGGTGCTGAGTTTTGAGCTGCTGAACGCTCCCGCTACAG
TGTGCGGACCCAAGCTCAGCACAGACCTGATCAAGAACCAGTGTGTGAACTTCGGA
GGTAGCGGCGGTTCTGGAAGAATGAAACAGATTGAGGATAAAATTGAAGAGATAT
TGTCAAAGATCTATCACATAGAGAACGAAATCGCCCGAATAAAGAAGCTGATTGG
GGAACGCTAGTGA (SEQ ID NO: 28034)
Group 3 3X RBD Sequence Codon Optimized Sequence; Kozak sequence in bold italic
GCCACCATGTTTGTCTTCCTGGTCTTGCTGCCGCTGGTGAGCAGCCGTGTGTCTCCC
ACCCAGGAAGTGATTAGATTTCCAAATATCACCAACCGCTGCCCTTTCGATAGGGT
GTTTAACGCCTCCAGGTTCCCAAGCGTCTACGCCTGGGAGAGAACCAAGATCAGCG
AATGTGTCGCCGACTACACAGTGCTGTATAATTCAACCTCTTTTTCCACATTCAAGT
GCTATGGAGTGTCACCATCTAAACTCATCGACTTATGTTTTACCAGCGTGTATGCAG
ACACCTTCTTGATCAGGTCCAGCGAGGTCAGACAGGTGGCACCTGGCGAAACCGGC
GTCATTGCTGACTACAATTACAAGCTGCCTGATGATTTTACCGGTTGCGTTATTGCC
TGGAACACAGCAAAACAGGATACCGGAAATTACTATTATCGTTCCCACCGGAAGA
CTAAGCTGAAACCCTTCGAGCGAGATCTTTCGTCAGACGACGGAAATGGGGTGTAC
ACACTTTCCACGTACGACTTCAATCCTAATGTGCCAGTGGCCTACCAGGCCACCAG
AGTCGTGGTGCTCTCATTTGAGCTGCTGAATGCCCCTGCTACAGTCTGTGGCCCCAA
ACTGTCCACTCAACTGGTGAAGAACCAGTGTGTGAACTTCGGAGGTAGCGGCGGTT
CTGGAGGCTACATTCCAGAAGCTCCACGCGATGGCCAGGCTTATGTGCGGAAAGAT
GGCGAGTGGGTGTTTCTCAGCACCTTTCTGAGTCCAGCTGGCGGTAGCGGCGGGGT
TCGGGCAGAGGGCAGAGGCTCACTGCTCACGTGTGGTGATGTAGAAGAGAATCCA
GGTCCTAGGAGGCGAGCACGGATGTTCGTATTTCTGGTGCTGCTGCCCCTAGTGAG
CTCCCGAGTTTCCCCCAGCACCGAGGTCATCAGGTTCCCTAACATCACCAACAGGT
GCCCTTTTGACAGAGTCTTCAACGCGTCTCGATTTCCGTCCGTGTACGCCTGGGAGC
GGACAAAGATTAGCGATTGTGTGGCCGACTACACTGTCCTCTACAATTCGACCAGC
TTTAGTACCTTCAAGTGCTATGGGGTCTCACCATCCAAACTAATTGATCTGTGTTTC
ACTTCTGTTTATGCGGATACTTTTCTCATAAGATTTTCGGAAGTACGACAGATCGCC
CCAGGCGAAACGGGAGTGATCGCTGACTACAACTATAAACTGCCCGACGAATTCA
CAGGATGCGTGATAGCTTGGAATACTGCCAACCAAGACAGGGGGCAGTATTACTA
CCGGTCCTCACGAAAAACGAAGTTGAAGCCCTTCGAGAGAGATTTGTCTAGCGACG
AAAATGGCGTTAGAACACTTTCAACCTATGACTTTTACCCTAGTGTGCCACTTGAGT
ATCAGGCTACCCGCGTGGTGGTCCTGAGCTTCGAGTTACTGAATGCACCTGCCACA
GTATGTGGTCCGAAGCTGTCTACCAGCCTCATTAAAAATCAGTGCGTGAATTTCGG
AGGTAGCGGCGGTTCTGGAGGGGGGTCTGGAGGCATAAAGGAAGAGATTGCCAAG
ATTAAGGAAGAACAGGCAAAGATAAAAGAAAAGATTGCAGAGATTGAAAAAAGG
ATTGCTGAGATCGAGAAAAGGATAGCAGGAGGTTGTTGCGGCGGTAGCGGCGGTG
TCCGTGCTGAAGGACGCGGGTCCCTTCTCACCTGCGGCGACGTTGAGGAAAACCCT
GGCCCTAGAAGGCGAGCACGGATGTTCGTGTTCCTGGTGCTTCTCCCTCTGGTGTCT
AGCAGGGTAGTGCCCAGTGGAGACGTGGTCCGGTTCCCTAACATCACCAACCTGTG
TCCCTTCGGGGAGGTGTTCAATGCCACCAAGTTCCCCAGTGTTTATGCATGGGAGA
GGAAGAAGATCTCCAACTGTGTGGCTGATTACTCTGTCCTGTACAACAGCACCTTC
TTCTCCACATTTAAATGTTATGGCGTTTCTGCTACCAAGCTGAATGACCTCTGCTTC
TCTAACGTGTATGCTGACAGTTTTGTGGTGAAGGGAGATGATGTTAGACAGATTGC
TCCTGGGCAGACCGGCGTCATTGCAGATTATAATTATAAACTCCCAGATGACTTCA
TGGGCTGCGTGCTCGCCTGGAATACCAGAAATATCGACGCTACTTCAACCGGAAAC
TACAATTACAAGTACCGGTACCTGAGACACGGGAAACTGAGACCATTCGAGCGGG
ACATTAGCAATGTGCCTTTCAGCCCCGATGGAAAGCCCTGCACTCCTCCTGCCCTG
AACTGCTACTGGCCCCTCAATGATTACGGCTTCTACACCACCACTGGAATTGGCTA
CCAGCCCTACAGAGTGGTTGTGCTGTCATTTGAGCTGCTGAACGCCCCAGCCACAG
TCTGTGGCCCCAAACTATCCACTGACCTGATTAAGAACCAGTGCGTGAACTTCGGA
GGTAGCGGCGGTTCTGGAAGAATGAAACAGATTGAGGATAAAATTGAAGAGATAT
TGTCAAAGATCTATCACATAGAGAACGAAATCGCCCGAATAAAGAAGCTGATTGG
GGAACGCTAGTGA (SEQ ID NO: 28035)
TCE12: TCE is shown in bold (990 amino acids); MAG is lowercase and class II helper
domain and linkers in italics
magYGDAVVYRGTTTYKLNVGDYFVLTSHIKWDLLKYDFTEERLKLFDLWVYKQ
FDTYNLWNTFTTSRYWEPEFYEAMYTPHRDAAMQRKLEKMADQAMTQMYKQA
RSEDKALLEDEFTPFDVVRQCSGVTVAGVSICSTMTNRQFHQKLLKSIAATRGAT
VVIGTSKFYGGWHNMLGVSFSTFEEAALCTFLLNKEMYLKLRSDKFPVLHDIGNP
KAIKCVPTANPKTPKYKFVRIQPGQTFSVLAGTGTSTDVVYRAFDIYNCVSFCYMH
HMELPTGVHAGTDLWPQIAQFAPSASAFFGMSRIGMECINANQVIVNNLDKSAGF
PFNKWGKARLYYMFYKGVITHDVSSAINRPQIGVFVNEFYAYLRKHFSMMILSDD
ATCRRCPAEIVDTVSALVYDNKLSPNLAWPLIVTALRANSNKTVGELGDVRETMS
YLFLYCQVHGNAHVASCDAIMTRCLAVDMTYRRLISMMGFKMNYQVNGFTQTT
ETAHSCNVNRENVAITRAKVGMKDLSPRWYFYYLGTGPTDGTLMIERFVSLAIDA
YPLTKHPNQEYADVFHLYLQYIRKLHDELTGHMLDMYSNVLFSTVFPPTSFGPLV
RKIFVDGVPFVVSTGYHFRELGVPLLESELVIGAVILRGHNNYMLTYNKVENMTP
RDLGACIDCGTTFTYASALWEIQQVVDADSKIWAQCVQLHNDILLAKDTTPGTGK
SHFAIGLALYYPSARIVYTACSHAAVDALCEKALTMLVKQGDDYVYLPYPDPSRIL
GAGCFAAYSRYRIGNYKLNTDHNLHSSRLSFKELLVYAADPAMHAASGNLLLDKR
TPRTLLTKGTLEPEYFNSVCRLMVKYLYFIKGLNNLNRGMVLGSLAISMATNYDL
SVVNARLRAKTLKNLSDRVVFVLWAHGFELTSMKYFVKIGPERAVSKGFFKEGSS
VELKHFFFAQDGNAAISDYDYYRYNLPTMCDIRQLLFVVEVVDKYCDGGSLYVNK
HAFHTPAGPGPGAKFVAAWTLKAAAGPGPGQYIKANSKFIGITELGPGPG (SEQ ID
NO: 28036)
TCE12 NUCLEOTIDE SEQUENCE (2970 bp)
TACGGGGACGCCGTGGTCTATAGGGGCACCACCACTTACAAACTGAATGTGGGAG
ACTATTTCGTGCTGACTAGCCACATCAAATGGGATCTGCTTAAGTACGACTTTACCG
AGGAGCGCCTGAAGCTGTTCGACCTCTGGGTGTATAAGCAGTTCGATACATACAAT
CTCTGGAATACATTCACTACATCTCGCTACTGGGAGCCTGAGTTCTACGAGGCTAT
GTACACCCCTCACAGAGACGCTGCTATGCAACGCAAGCTAGAGAAGATGGCCGAC
CAGGCAATGACCCAGATGTACAAGCAGGCTCGGAGCGAGGACAAGGCCCTGCTTG
AGGACGAGTTTACACCTTTTGATGTGGTGCGGCAGTGTAGCGGCGTGACAGTGGCA
GGCGTGTCAATTTGTAGCACCATGACCAACAGACAGTTCCACCAGAAGCTGCTGAA
AAGCATCGCAGCTACAAGGGGCGCCACCGTTGTTATTGGCACATCCAAATTCTACG
GAGGCTGGCACAATATGCTCGGCGTTAGCTTTTCAACCTTTGAGGAGGCCGCCTTG
TGTACTTTCCTTCTCAATAAGGAGATGTATTTGAAACTCAGGTCTGATAAATTCCCA
GTGCTGCACGACATAGGAAATCCTAAAGCTATCAAGTGCGTACCTACTGCTAACCC
CAAGACCCCTAAATATAAGTTCGTGAGGATCCAGCCTGGTCAGACATTTAGCGTCC
TCGCTGGTACAGGTACTAGCACCGATGTTGTCTACAGAGCCTTCGACATCTACAAT
TGCGTGAGCTTTTGTTATATGCACCACATGGAGCTCCCAACTGGAGTGCATGCCGG
CACCGACCTGTGGCCCCAGATCGCACAGTTTGCACCTTCAGCTTCCGCATTTTTCGG
AATGAGCCGCATCGGCATGGAATGTATTAATGCCAACCAGGTCATTGTCAATAACC
TCGATAAGTCTGCCGGATTCCCCTTCAATAAATGGGGAAAGGCCCGCCTGTATTAT
ATGTTCTATAAGGGGGTCATTACTCATGATGTCAGCTCCGCTATTAATAGGCCTCAG
ATCGGCGTGTTCGTCAACGAATTTTATGCTTACCTTAGGAAACACTTCAGTATGATG
ATCCTCAGCGACGATGCAACATGCCGCAGATGCCCCGCCGAAATTGTCGACACAGT
CAGCGCTCTCGTCTACGACAACAAGCTGTCCCCCAACCTCGCCTGGCCCCTCATCGT
GACCGCCCTGAGGGCCAACAGCAACAAGACTGTGGGGGAACTGGGCGACGTGAGA
GAGACCATGAGCTACTTATTTCTGTACTGTCAGGTGCACGGCAATGCCCACGTGGC
TAGCTGCGACGCCATTATGACACGGTGCCTGGCTGTGGACATGACTTACAGACGGC
TCATCTCTATGATGGGATTTAAGATGAACTACCAGGTGAATGGGTTCACACAAACA
ACAGAAACAGCCCATAGCTGTAATGTCAATCGGTTTAACGTTGCAATCACTCGCGC
CAAAGTGGGAATGAAAGATCTTTCCCCAAGGTGGTATTTCTATTACCTGGGAACCG
GCCCTACCGATGGAACGTTGATGATTGAAAGGTTTGTGTCCCTAGCCATTGATGCC
TATCCGCTGACCAAGCACCCAAATCAGGAATACGCTGACGTTTTCCACCTCTATCTC
CAGTACATCAGAAAATTGCACGATGAGCTCACCGGGCATATGCTTGACATGTATAG
CAACGTTCTGTTTAGTACAGTTTTTCCTCCCACCAGCTTCGGGCCCCTGGTGAGAAA
AATCTTTGTCGATGGCGTTCCTTTTGTGGTGAGCACGGGCTATCATTTCCGGGAATT
AGGCGTCCCTCTGCTGGAGTCCGAGCTGGTGATCGGAGCCGTGATCCTGAGAGGCC
ACAACAATTACATGCTGACCTACAACAAAGTTGAGAACATGACACCACGGGACCTT
GGAGCATGTATTGACTGCGGAACTACGTTTACTTATGCCTCTGCACTTTGGGAAATT
CAGCAGGTTGTGGATGCCGATTCTAAAATCTGGGCCCAGTGTGTGCAGCTGCACAA
TGATATTCTTCTGGCCAAGGATACAACTCCTGGGACTGGAAAGTCCCACTTTGCCA
TCGGGCTGGCCCTGTACTATCCATCAGCACGGATTGTGTATACTGCCTGCTCTCACG
CCGCAGTGGACGCCCTGTGTGAAAAGGCACTGACCATGCTCGTGAAACAGGGAGA
TGACTATGTGTATCTGCCATACCCAGACCCTTCTCGGATCTTGGGCGCTGGATGTTT
TGCAGCCTACAGTCGGTATAGAATTGGCAATTATAAGCTGAATACCGATCACAACC
TTCATAGCTCCAGGCTGTCATTTAAGGAACTGCTGGTCTACGCCGCCGACCCAGCA
ATGCACGCTGCCTCCGGCAACCTCCTCCTTGACAAGAGAACTCCTAGGACTCTGCT
CACCAAGGGCACCCTGGAGCCAGAGTACTTCAACTCTGTGTGTCGCCTTATGGTGA
AGTATCTCTACTTCATTAAGGGCCTGAACAATCTTAACCGCGGAATGGTGCTGGGC
TCCCTGGCCATTAGTATGGCCACCAATTATGATCTCTCAGTGGTGAACGCCAGACT
GAGAGCTAAGACCCTGAAGAACTTGAGCGATAGAGTGGTGTTTGTGCTGTGGGCTC
ATGGATTTGAATTGACCAGCATGAAATACTTCGTGAAGATCGGGCCAGAGAGGGC
CGTGTCTAAAGGCTTCTTCAAAGAGGGAAGCAGCGTAGAACTGAAACATTTCTTCT
TTGCTCAGGACGGAAACGCGGCTATTAGCGACTACGACTACTACAGATATAATCTG
CCCACAATGTGCGATATCCGGCAGCTCCTGTTTGTAGTGGAGGTGGTGGATAAATA
CTGTGACGGCGGATCCCTGTATGTGAATAAGCACGCCTTCCACACTCCAGCC (SEQ
ID NO: 28037)
TCE13: TCE is shown in bold (781 amino acids); MAG is lowercase and class II helper
domain and linkers in italics.
magYGDAVVYRGTTTYKLNVGDYFVLTSHKHFFFAQDGNAAISDYDYYRYNLPTW
PQIAQFAPSASAFFGMSRIGMECINANQVIVNNLDKSAGFPFNKWGKARLYYTSRY
WEPEFYEAMYTPHRDAAMQRKLEKMADQAMTQMYKQARSEDKPGTGKSHFAI
GLALYYPSARIVYTACSHAAVDALCEKALTMLVKQGDDYVYLPYPDPSRILGAGC
FGTTFTYASALWEIQQVVDADSKIFTQTTETAHSCNVNRFNVAITRAKVGMKDLS
PRWYFYYLGTGPTDGTLMIERFVSLAIDAYPLTKHPNQEYADVFHLYLQYIRKLH
DELTGHMLDMYSNVLFSTVFPPTSFGPLVRKIFVDGVPFVVSTGYHFRELGVPLLE
SELVIGAVILRGHNNYMLTYNKVENMTPRDLGACIDCSDRVVFVLWAHGFELTS
MKYFVKIGPERFVNEFYAYLRKHFSMMILSDDATCRRCPAEIVDTVSALVYDNKL
SPNLAWPLIVTALRANSNKTVGELGDVRETMSYLFLWVYKQFDTYNLWNTFTAL
LEDEFTPFDVVRQCSGVTVAGVSICSTMTNRQFHQKLLKSIAATRGATVVIGTSKF
YGGWHNMLAAYSRYRIGNYKLNTDHNLHSSRLSFKELLVYAADPAMHAASGNLL
LDKRTLYCQVHGNAHVASCDAIMTRCLAVDMTYRRLISMMGFKMNYQVNGDKF
PVLHDIGNPKAIKCVPTANPKTPKYKFVRIQPGQTFSVLAGTGTSTDVVYRAFDIY
NCDGGSLYVNKHAFHTPAGPGPGAKFVAAWTLKAAAGPGPGQYIKANSKFIGITELGPG
PG (SEQ ID NO: 28038)
TCE13 NUCLEOTIDE SEQUENCE (2343 bp)
TATGGAGATGCAGTTGTGTACAGAGGCACCACCACATACAAACTCAATGTGGGGG
ATTATTTCGTGCTGACCTCCCACAAGCACTTTTTCTTTGCACAGGACGGAAATGCCG
CTATCTCCGACTATGATTACTACCGGTACAATTTACCCACCTGGCCTCAGATTGCCC
AGTTTGCCCCAAGCGCCAGCGCCTTCTTCGGCATGAGCAGGATCGGGATGGAGTGT
ATTAATGCTAACCAAGTCATTGTGAACAATTTGGACAAGTCAGCCGGATTCCCATT
CAACAAATGGGGCAAAGCTCGGTTATATTACACATCTCGCTACTGGGAACCAGAGT
TCTATGAGGCAATGTATACTCCACATAGGGATGCTGCCATGCAGAGAAAGCTTGAG
AAGATGGCGGATCAGGCCATGACCCAGATGTATAAACAGGCCAGAAGCGAAGATA
AACCTGGCACTGGCAAGAGTCACTTCGCCATCGGACTGGCCCTCTACTACCCTAGC
GCTAGAATTGTTTATACCGCTTGTTCTCACGCAGCCGTGGATGCCCTGTGTGAGAA
GGCCCTGACTATGCTGGTGAAACAAGGGGACGATTACGTGTACCTGCCTTACCCCG
ACCCATCCAGGATCCTGGGAGCCGGCTGCTTCGGAACCACCTTCACCTATGCTTCC
GCCTTGTGGGAGATTCAGCAGGTGGTGGACGCCGACTCTAAGATTTTCACTCAGAC
CACTGAAACAGCTCACTCTTGCAACGTCAATCGCTTTAACGTTGCAATAACCAGGG
CCAAGGTAGGAATGAAGGATCTATCACCCCGGTGGTATTTTTACTATCTGGGGACA
GGCCCCACAGACGGAACACTGATGATAGAAAGATTCGTGTCCCTGGCCATTGACGC
CTACCCTCTGACTAAGCACCCTAATCAGGAATATGCTGATGTGTTCCATTTGTACCT
TCAATACATCCGGAAGCTGCACGACGAGCTGACTGGACACATGCTCGACATGTACA
GTAACGTTCTCTTCAGCACAGTCTTCCCACCCACATCTTTTGGACCTCTGGTGCGCA
AGATCTTTGTCGATGGGGTCCCTTTCGTGGTTAGCACTGGATACCACTTCCGGGAAT
TGGGGGTGCCACTGCTGGAGAGCGAGCTTGTTATCGGGGCCGTGATTCTGCGCGGA
CACAATAACTACATGCTGACTTACAATAAAGTGGAGAACATGACACCTCGGGATCT
GGGCGCATGTATCGACTGCTCAGATCGAGTGGTCTTTGTGTTATGGGCCCACGGCT
TTGAACTGACAAGCATGAAATACTTTGTGAAAATCGGGCCTGAACGGTTCGTGAAC
GAATTCTACGCATATCTGAGAAAACACTTCTCTATGATGATCCTGAGCGATGACGC
CACCTGCAGGCGGTGTCCAGCTGAGATTGTGGACACCGTGTCCGCACTGGTGTACG
ATAACAAGCTGAGTCCCAACCTAGCCTGGCCCCTGATTGTGACAGCCCTCAGGGCT
AATTCAAATAAAACTGTGGGCGAGCTCGGCGACGTGAGAGAGACCATGTCCTACCT
GTTCCTGTGGGTGTACAAGCAATTCGATACCTACAACCTGTGGAATACATTTACCG
CTCTGCTCGAGGATGAGTTCACTCCCTTTGATGTTGTGAGACAGTGTTCTGGCGTGA
CTGTGGCAGGAGTGAGCATCTGTAGCACAATGACCAATCGGCAGTTCCACCAGAA
GTTATTGAAGTCCATCGCCGCCACTAGGGGAGCCACCGTGGTGATCGGAACATCAA
AGTTTTATGGAGGCTGGCACAACATGCTTGCCGCCTACAGTAGATATCGTATTGGC
AACTATAAACTGAACACAGATCACAATCTGCACTCGAGCAGACTGTCTTTTAAGGA
GCTGCTCGTCTATGCCGCCGATCCCGCAATGCACGCCGCAAGTGGTAATCTGCTCC
TGGATAAAAGAACCCTGTACTGTCAGGTTCATGGCAACGCTCACGTGGCATCCTGT
GATGCCATCATGACTAGGTGTCTGGCCGTTGACATGACCTATAGACGCCTGATCAG
CATGATGGGATTTAAGATGAACTACCAGGTGAACGGCGACAAGTTCCCTGTCCTGC
ATGATATCGGTAACCCAAAGGCCATCAAATGTGTGCCTACCGCCAACCCCAAGACA
CCTAAGTATAAGTTTGTAAGGATCCAGCCTGGTCAAACTTTTAGTGTTTTAGCCGGG
ACCGGCACCAGTACTGATGTAGTGTATAGGGCTTTTGACATCTATAACTGTGACGG
AGGTAGCCTCTATGTGAATAAACATGCCTTTCATACACCAGCC (SEQ ID NO: 28039)
TCE14: TCE is shown in bold (598 amino acids); MAG is lowercase and class II helper
domain and linkers in italics.
magYGDAVVYRGTTTYKLNVGDYFVLTSHMKDLSPRWYFYYLGTGPTDGTLMIE
RFVSLAIDAYPLTKHPNQEYADVFHLYLQYIRKLHDELTGHMLDMYSFYAYLRK
HFSMMILSDDLWVYKQFDTYNLWNTFTMQRKLEKMADQAMTQMYKQARSEDK
PGTGKSHFAIGLALYYPSARIVYTACSHATSRYWEPEFYEAMYTPHCINANQVIVN
NLDKSAGFPFNKWGKARLYYALLEDEFTPFDVVRQCSGVTVAGVSICSTMTNRQF
HQKLLKSIAATRGATVVIGTSKFYGGWHNMLTMLVKQGDDYVYLPYPDPSRILG
AGCFAAYSRYRIGNYKLNTDHNLHSSRLSFKELLVYAADPAMHAASGNLLLDKRT
VRKIFVDGVPFVVSTGYHFRELGVPLLESELVIGAVILRGHNNYMLTYNKVENMT
PRDLGACIDCSDRVVFVLWAHGFELTSMKYFVKIGPERKHFFFAQDGNAAISDYD
YYRYNLPTWPQIAQFAPSASAFFGMSRIGMETCRRCPAEIVDTVSALVYDNKLTAN
PKTPKYKFVRIQPGQTFSVLAGTGTSTDVVYRAFDIYNCDGGSLYVNKHAFHTPA
GPGPGAKFVAAWTLKAAAGPGPGQYIKANSKFIGITELGPGPG (SEQ ID NO: 28040)
TCE14 NUCLEOTIDE SEQUENCE (1794 bp)
TACGGCGATGCCGTCGTGTATAGAGGAACAACCACCTATAAGCTTAATGTGGGAGA
TTATTTCGTGCTTACTAGTCATATGAAGGACCTGTCTCCTAGGTGGTACTTCTATTA
CCTGGGCACCGGCCCAACTGACGGCACCCTGATGATCGAGAGGTTTGTGAGCCTGG
CCATTGATGCTTATCCACTCACCAAGCACCCCAACCAGGAGTATGCCGATGTTTTCC
ACCTCTACCTGCAGTACATTAGAAAGCTCCATGATGAGTTAACAGGCCACATGCTT
GACATGTATAGCTTTTATGCCTATCTGAGAAAGCACTTTAGTATGATGATTCTGAGT
GATGACCTCTGGGTGTATAAACAGTTCGACACCTACAATCTGTGGAACACCTTCAC
TATGCAGAGAAAACTGGAGAAGATGGCCGATCAGGCTATGACTCAGATGTACAAG
CAGGCCCGGTCTGAGGACAAGCCAGGGACAGGCAAAAGCCACTTTGCAATAGGGT
TGGCCTTGTACTATCCTAGTGCCAGAATCGTGTACACCGCCTGCTCCCATGCTACCT
CAAGGTATTGGGAGCCCGAGTTCTACGAGGCAATGTATACCCCTCATTGTATCAAT
GCAAATCAGGTTATCGTAAACAATCTCGATAAGAGCGCAGGCTTCCCCTTTAACAA
ATGGGGGAAGGCTCGGCTGTACTACGCACTCCTGGAAGACGAGTTTACACCTTTTG
ACGTGGTGAGACAGTGCAGCGGGGTGACAGTTGCCGGCGTGTCTATCTGCAGCACC
ATGACTAATAGGCAGTTTCATCAGAAGCTTCTGAAATCTATCGCCGCCACCAGAGG
TGCCACTGTGGTGATCGGTACCTCTAAGTTTTACGGCGGCTGGCATAATATGCTTAC
AATGCTGGTGAAGCAGGGGGACGATTACGTGTATCTGCCTTACCCAGACCCTTCTC
GTATTCTGGGAGCTGGCTGCTTCGCTGCATATTCCAGATACCGCATTGGCAATTACA
AGCTGAACACAGATCATAATCTGCACTCTTCAAGATTGTCCTTCAAGGAACTTCTG
GTGTATGCTGCAGATCCCGCCATGCACGCTGCCTCCGGGAACCTGCTGCTGGACAA
GAGAACTGTGAGGAAGATTTTTGTCGATGGAGTGCCATTCGTGGTGTCCACTGGAT
ACCATTTCAGAGAGCTCGGCGTGCCTCTTCTGGAGTCCGAGCTGGTTATTGGCGCC
GTGATCCTTCGGGGCCACAATAACTACATGCTGACATACAATAAAGTGGAGAACAT
GACACCTCGGGACTTAGGCGCTTGTATTGACTGCAGCGACCGGGTGGTGTTTGTGC
TGTGGGCACACGGCTTCGAGCTGACCAGCATGAAATATTTTGTGAAGATCGGACCC
GAGAGGAAACATTTCTTCTTCGCCCAGGACGGCAATGCCGCCATCTCCGACTATGA
TTATTACCGGTACAACCTCCCCACCTGGCCTCAGATTGCCCAGTTCGCCCCTTCTGC
CAGCGCTTTCTTCGGCATGAGCCGGATTGGCATGGAGACATGCAGGAGATGTCCTG
CAGAAATCGTGGATACAGTGTCTGCCCTGGTCTATGATAACAAGCTCACTGCCAAC
CCAAAGACCCCCAAATACAAGTTCGTGAGGATCCAGCCCGGCCAGACATTTTCTGT
GCTGGCCGGAACTGGGACATCCACCGATGTGGTGTACCGCGCATTTGATATCTATA
ATTGTGACGGAGGCAGCCTGTATGTGAATAAGCACGCCTTCCACACACCTGCC (SEQ
ID NO: 28041)
TCE15: TCE is shown in bold (385 amino acids); MAG is lowecase and class II helper domain
and linkers in italics.
magWPQIAQFAPSASAFFGMSRIGMEDAYPLTKHPNQEYADVFHLYLQYIRKLHDE
LTGHMLDMYSFYAYLRKHFSMMILSDDPGTGKSHFAIGLALYYPSARIVYTACSH
ATSRYWEPEFYEAMYTPHCDGGSLYVNKHAFHTPANNLDKSAGFPFNKWGKART
YNKVENMTPRDLGACIVAGVSICSTMTNRQFHQKLLKSIAATRGATVVIGTSKFY
GGWHNMLTMLVKQGDDYVYLPYPDPSRILGAGCFAAYSRYRIGNYKLNTDHNLH
SSRLSFKELLVYAADPAMHAASGNLLLDKRTVRKIFVDGVPFVVSTGYHFRELGV
PLLESELVIGAVILRGHKHFFFAQDGNAAISDYDYYRYNLPTMKDLSPRWYFYYL
GTGPGPGPGAKFVAAWTLKAAAGPGPGQYIKANSKFIGITELGPGPG (SEQ ID NO: 28042)
TCE15 NUCLEOTIDE SEQUENCE (1155 bp)
TGGCCTCAGATCGCACAGTTTGCTCCTTCTGCCAGCGCCTTCTTTGGCATGTCCAGG
ATAGGAATGGAAGATGCCTATCCACTGACCAAGCACCCTAACCAGGAATATGCAG
ATGTCTTCCATCTCTACCTGCAGTACATCAGAAAACTGCATGACGAGCTGACAGGA
CACATGTTAGACATGTACTCATTTTACGCCTATCTCAGGAAACATTTTTCCATGATG
ATCCTGTCTGACGACCCCGGCACAGGAAAGAGCCATTTCGCTATTGGCCTGGCTCT
GTACTACCCCAGCGCTAGAATCGTGTACACAGCCTGCTCCCACGCCACTAGCCGGT
ATTGGGAGCCAGAGTTCTATGAGGCCATGTATACACCTCACTGCGATGGAGGAAGT
CTGTATGTTAACAAGCATGCCTTCCACACACCTGCCAATAATCTGGATAAGTCCGC
TGGATTTCCCTTTAATAAGTGGGGCAAAGCTAGAACTTACAATAAAGTGGAAAACA
TGACACCCAGGGACTTAGGCGCCTGCATCGTGGCCGGCGTGAGCATCTGCTCAACC
ATGACAAATAGACAGTTCCACCAGAAGTTGTTGAAATCTATCGCCGCAACCAGGGG
GGCCACCGTGGTGATCGGCACCTCTAAGTTTTATGGCGGCTGGCACAACATGCTGA
CCATGCTTGTGAAGCAGGGAGATGATTACGTGTACCTGCCCTACCCAGATCCTAGC
AGAATCCTGGGCGCCGGCTGCTTTGCCGCTTACTCTAGGTACAGAATCGGCAACTA
CAAGCTGAATACAGATCACAATCTCCACTCATCTAGACTGTCCTTTAAGGAGCTGC
TGGTGTACGCCGCTGATCCAGCCATGCACGCCGCCAGCGGGAATCTCCTGCTTGAT
AAAAGGACAGTGAGAAAGATCTTTGTGGATGGCGTGCCTTTCGTGGTGTCCACAGG
CTATCACTTTAGGGAGCTGGGAGTTCCTCTGCTGGAGAGCGAGCTGGTCATCGGCG
CCGTGATTCTGAGAGGCCACAAACACTTTTTCTTCGCCCAGGATGGAAATGCCGCC
ATCTCCGATTACGATTATTATAGATACAACCTGCCTACAATGAAGGACCTGAGCCC
CAGATGGTACTTCTACTACCTGGGCACCGGCCCT (SEQ ID NO: 28043)

### XIV.M. Multi-RBD Vaccine Construction

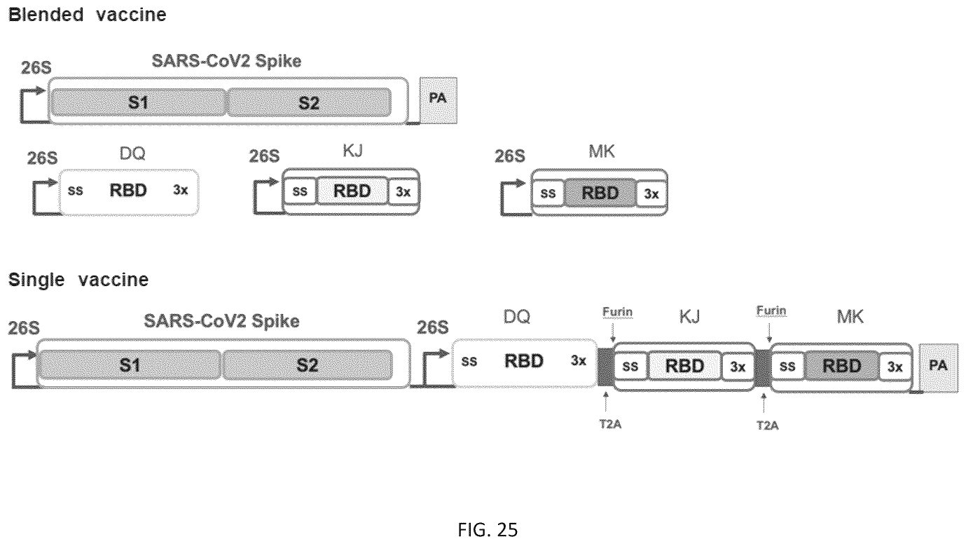

Vaccines using selected group-specific RBDs as components together with a SARS-CoV-2 Spike protein (SARS-COV2-Delta Spike-F2P) were constructed to assess immunogenicity. As shown in FIG. 25, vaccines were designed as either “blended” vaccines as a combination of four single SAM vectors, three expressing unique RBDs and the fourth expressing a whole SARS-COV2 Spike (FIG. 25; top), or as a single vector expressing a full-length SARS-COV2 Spike plus multiple RBDs (FIG. 25; bottom).

### XIV.N. Multi-RBD Expression Assessment

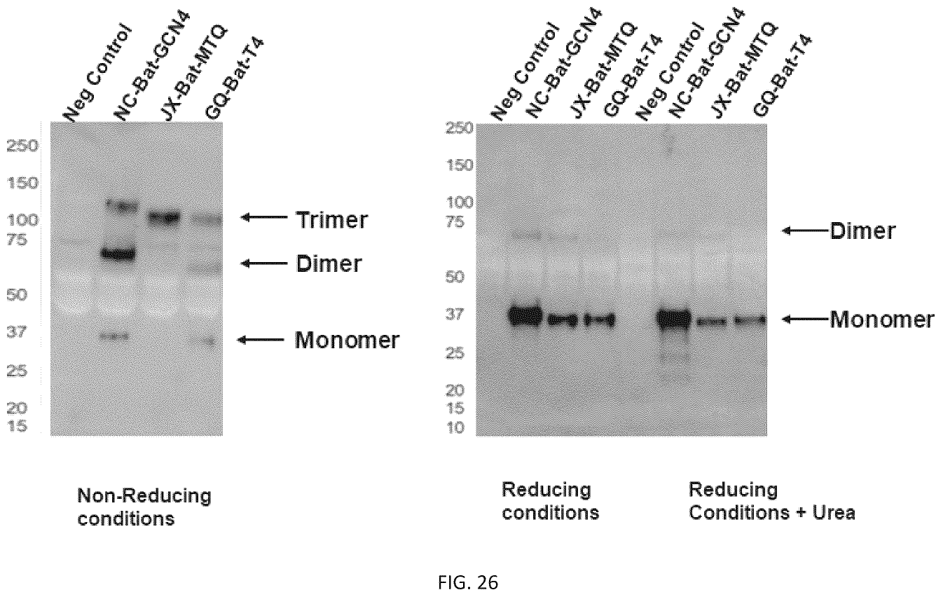

RBD expression was evaluated and confirmed by Western analysis using both anti-SARS-COV-2 polyclonal antibody (FIG. 26) or using an anti-His antibody (not shown). As a representative experiment, group 2 RBDs (RBDΔRBM) expressed 72h post transfection from individual SAMs is shown in FIG. 26. RBD sizes indicative of trimerized proteins (top bands) was observed when SAM transfected lysates were run under non-reducing conditions (FIG. 26; left panel) indicating that the RBDS were secreted and in the expected format consistent with trimerization (which may present the RBD in a more natural format for optimal immunogenicity, e.g., to elicit antibodies against non-linear/tertiary epitopes). RBD expression was confirmed for all RBDs and trimerization combinations.

### XIV.O. In Vivo Evaluation of SARS-COV-2 Spike and Multi-RBD Cassette Design in Naïve Mice (Blended Vaccines)

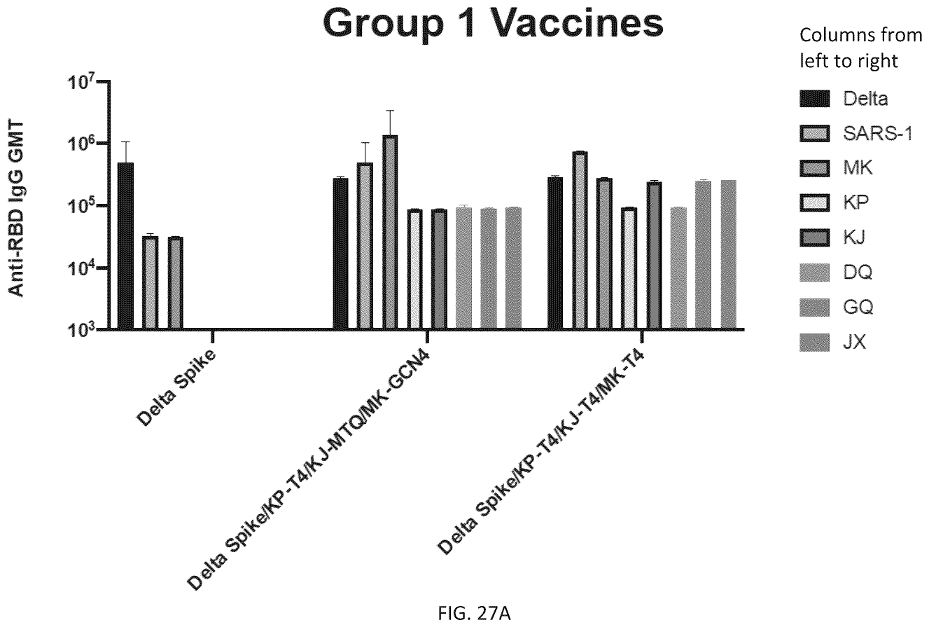

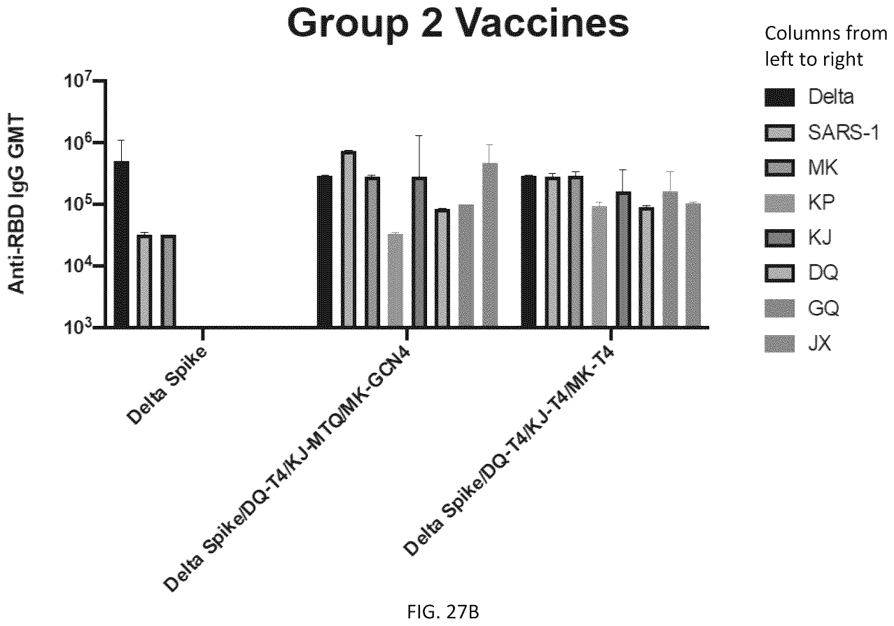

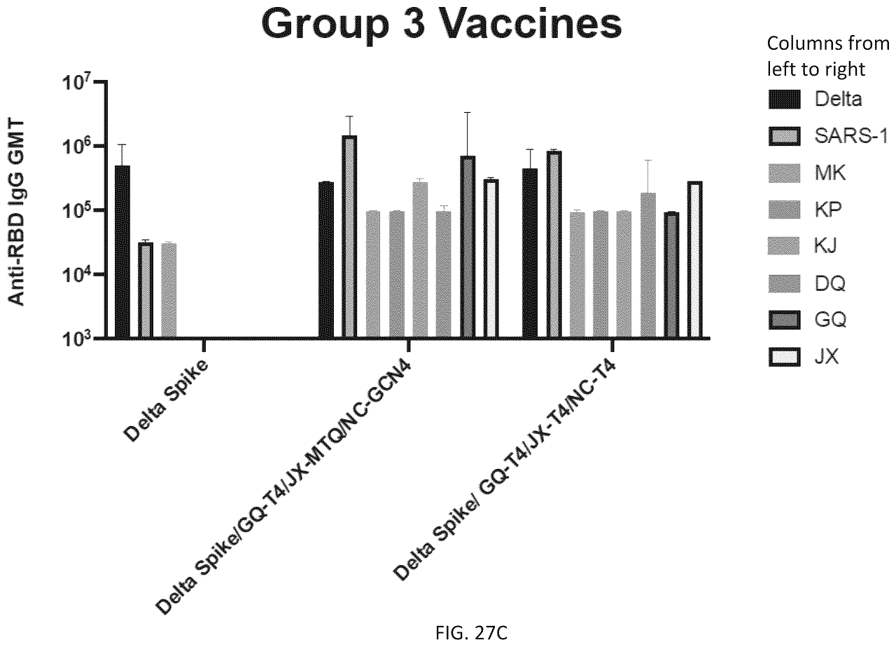

“Blended” samRNA vectors were tested at a dose of 3 μg/SAM, four SAMs per vaccine group each coding for 1 of 3 unique RBDs and the fourth SAM expressing a full-length Delta variant Spike. A delta Spike SAM alone (“Delta Spike”) and each RBD alone (data not shown) were used as control SAM vaccines were also assessed. Vaccine blends were tested where each RBD had the same T4 trimerization domain or a unique domain. Shown in FIG. 27 are anti-RBD antibody responses to vaccine matched (grey or black bars) and unmatched RBDs (color bars) measured at 4 wk post prime for Group 1 (FIG. 27A; MK/KP/KJ), Group 2 (FIG. 27B; DQ/KJ/MK), and Group 3 (FIG. 27C; GQ/JX/NC) vaccines. Vaccines with either the T4 Trimerization domain (right series) or unique domains (middle series) induced potent antibody responses to all RBDs tested, while a Delta Spike SAM only (left series) induced strong antibody responses to Delta Spike and lower cross-reactivity to SARS-1 and MK RBDs only.

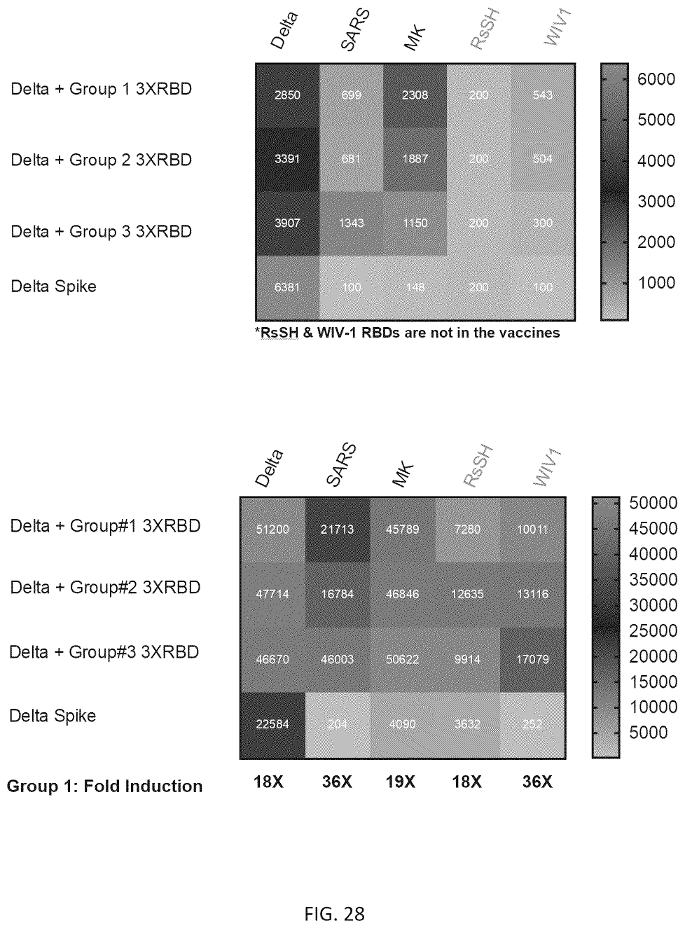

Neutralizing immunity was measured using a PNA assay at 4 weeks post prime (FIG. 28; top panel) and at 4 weeks post boost (FIG. 28; bottom panel). PNA titers indicated strong responses against pseudoviruses which are directly matched to the vaccines (Delta, SARS-1, Bat-MK) and lower cross-reactivity to WIV-1 that is more closely matched to SARS-1 (sharing 94.6% amino acid identity). The Bat species RsSHC014 is more distantly related to all the vaccine delivered RBDs and Spike protein with the closest RBD, SARS-1, having only 82% identity, and failed to show a neutralizing titer above 200 (PNA background for this virus).

Priming with the same groups of SAM vectors resulted in a potent boost in neutralizing titers with fold inductions being in the order of 18-36× for the group 1 vaccine blend. Notably, the boost increased not only the magnitude of the response but also the breadth of the immune response as following the boost RsSHC014 demonstrated PNA titers above background of 7280, 12,635 and 9914 for Groups 1, 2 and 3 blended vaccines, respectively.

### XIV.P. In Vivo Evaluation of SARS-COV-2 Spike and Multi-RBD Cassette Design in Non-Naïve Mice (Blended Vaccines)

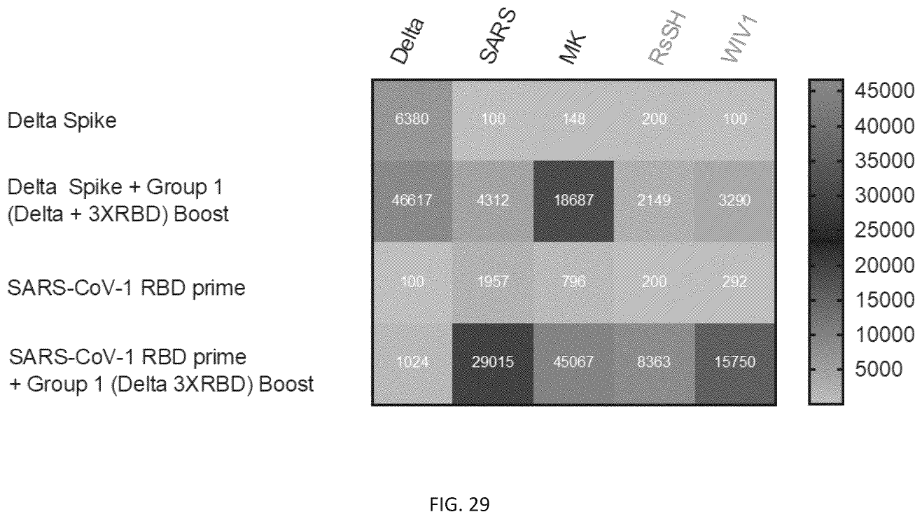

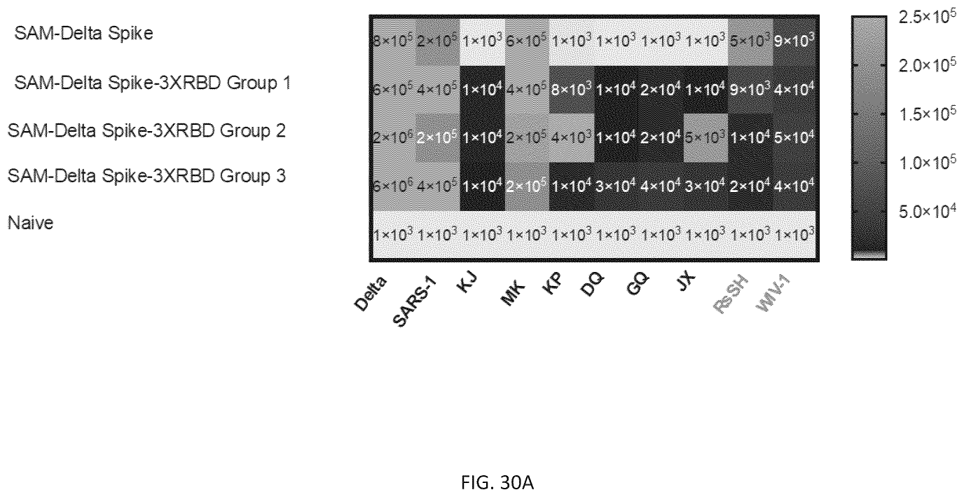

To investigate whether prior immunity to coronaviruses, either through natural infection or through vaccination, may boost the potency of a pan-sarbecovirus vaccine, blended vaccines were evaluated in mice vaccinated with either SARS-COV-2 Spike or a SARS-COV-1 (SARS-1) RBD. Groups of mice were first immunized with 3 μg of a SAM expressing cither the full-length Delta Spike or SARS-COV-1 RBD. The Delta and SARS-1 primed groups were then boosted with the group 1 vaccine blend and immunity was compared to the pre-boosted group. As shown in FIG. 29, boosting with the group 1 vaccine increased both the magnitude and breadth of the immune responses.

XIV.Q. In Vivo Evaluation of SARS-COV-2 Spike and Multi-RBD Cassette Design in Mice with Single Vaccines Homologous samRNA Prime/Boost

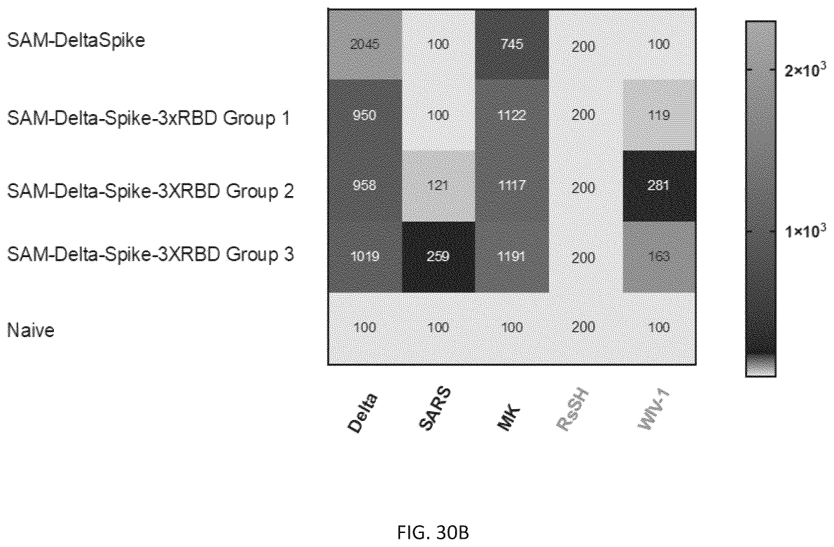

samRNA vectors expressing a full-length Spike and three RBDs on the same SAM backbone were evaluated and compared to a control SAM expressing full length Delta Spike. Shown in FIG. 30 is anti-Spike and RBD data at 4 weeks post prime as assessed by anti-RBD antibody titers (FIG. 30A) and a neutralizing antibody PNA assay (FIG. 30B). Each single SAM multi-Spike/RBD vaccine generated broad immunity to multiple RBDs, even those that were not matched to the respective vaccine. Antibody titers were also observed against RsSHC014 and WIV1 (FIG. 30A). PNA titers (FIG. 30B) were lower than those observed the blended vaccines and were mainly to Delta Spike and Bat MK RBD for all vaccine groups. The group 3 single vector also generated a noticeable response to SARS-1. Group 2 and 3 vectors also generated PNA titers against WIV-1. No groups generated PNA titers against RsSH.

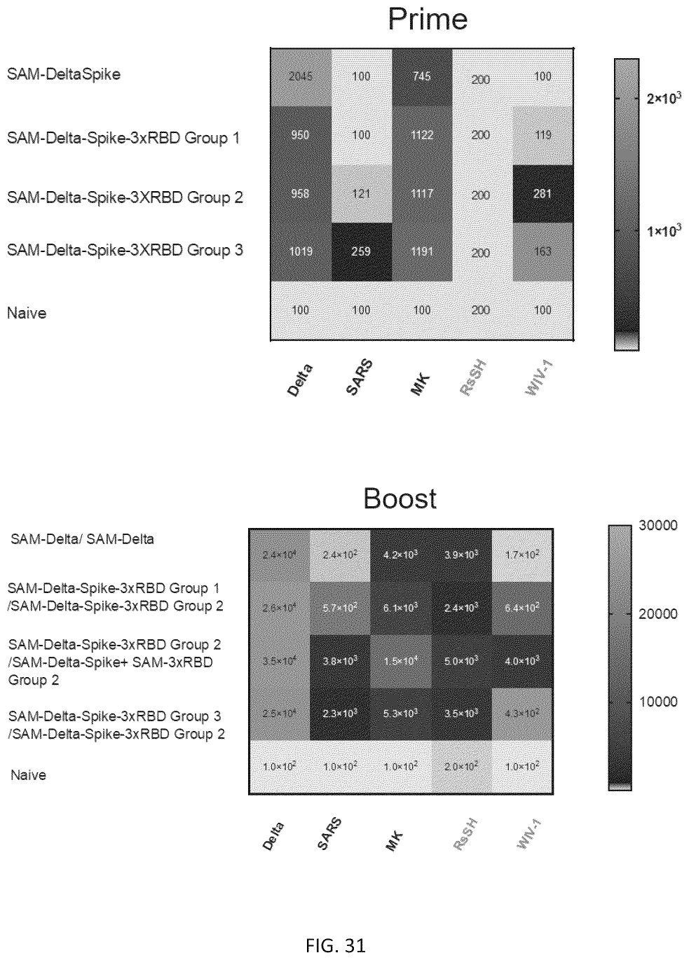

Groups 1-3 were then boosted with Group 2 RBD vaccines. As shown in FIG. 31, PNA titers noticeably increased post boost (bottom panel) compared to post prime (top panel).

XIV.R. In Vivo Evaluation of SARS-COV-2 Spike and Multi-RBD Cassette Design in Mice with Single Vaccines Heterologous Prime/Boost

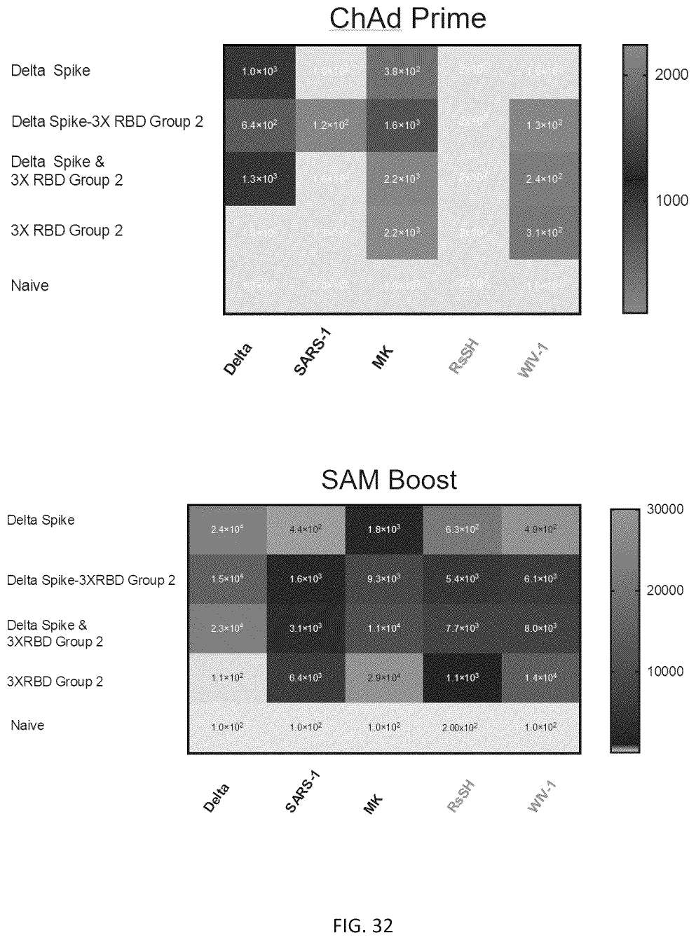

A ChAd vector expressing a full-length SARS-COV2 Spike and 3×-RBDs on the same backbone was compared to a blend of two SAM vectors: one expressing a SARS-COV-2 Spike and another expressing Group 2 3×-RBD. Vectors expressing Group 2 3×-RBDs or SARS-COV-2 were used as controls. As shown in FIG. 32 (top panel), a priming boost with the ChAd single vector expressing both the full-length Spike and the 3×-RBDs induced PNA titers against SARS-COV-2, SARS-1, Bat MK and Bat WIV-1, but not RsSH. As shown in FIG. 32 (bottom panel), boosting with the corresponding SAM vectors increased PNA titers and for the single vector titers were increased to between 1.6e3 to 1.5e4 for the different pseudoviruses assessed.

### XIV.S. In Vitro Evaluation of TCE Cassette Expression

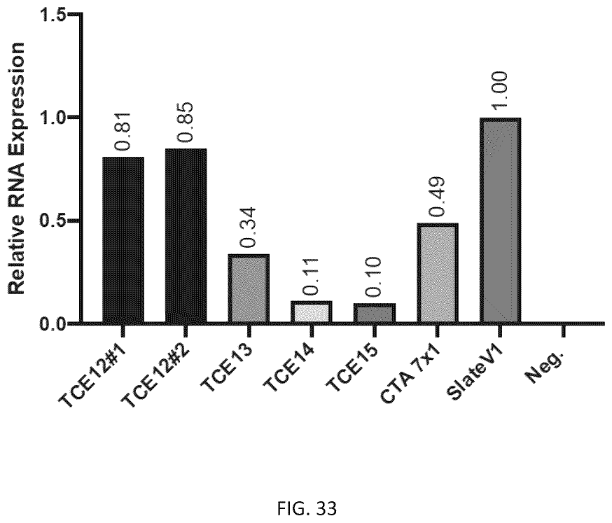

Expression of TCEs was performed using a RT-qPCR approach using primers and a probes specific for the class II PADRE domain. TCEs were evaluated initially using ChAd vectors. Four TCEs were cloned into ChAd68 and the virus for the different TCEs were made. The virus was used to infect 293F cells and RNA was harvested and used to make cDNA. The cDNA was then evaluated for PADRE expression and levels were measured relative to a Beta-actin housekeeping gene. Shown in FIG. 33 are the relative RNA values normalized to an oncology vector. TCE12 had 81-85% expression of Slate VI and was approximately 2.4× greater than TCE13 and 8× greater than TCE14 and 15.

XIV.O. In Vivo Evaluation of Vaccines with TCE Cassettes

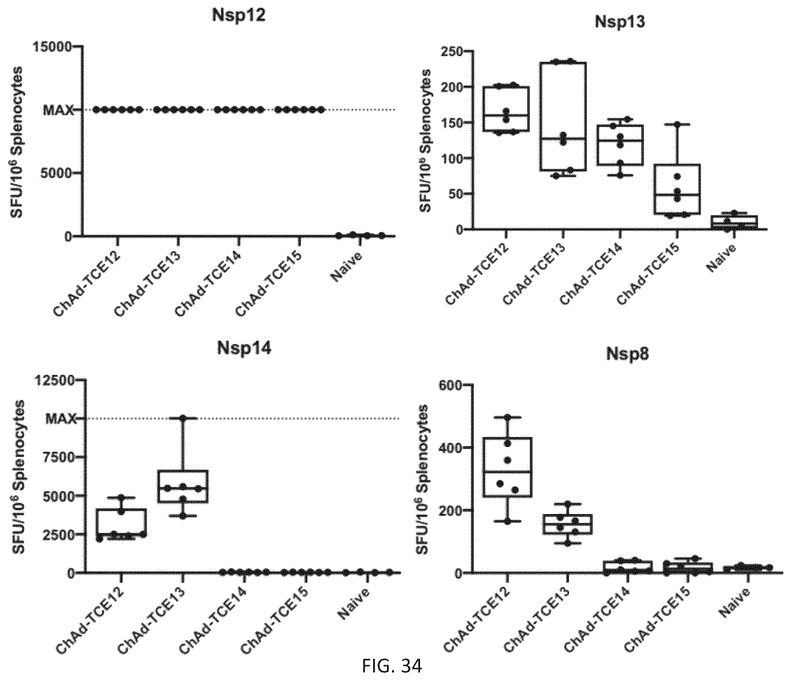

ChAd vectors expressing the various TCEs (TCE12, TCE13, TCE14, and TCE15) were evaluated in C57/Bl6 mice at 14 days post vaccination. Immune responses were measured against multiple SARS-COV-2 peptide pools. Overlapping (15mers over lapping by 11mers) peptide pools covering the various TCE antigen hot spots were made and evaluated by ELISpot. As shown in FIG. 34, strong T-cell responses were observed to Nsp 12 for all TCEs (top left) and to Nsp 14 for TCE12 and TCE13 only (bottom left). Lower but detectable responses were observed for Nsp13 and Nsp8 for all TCEs.

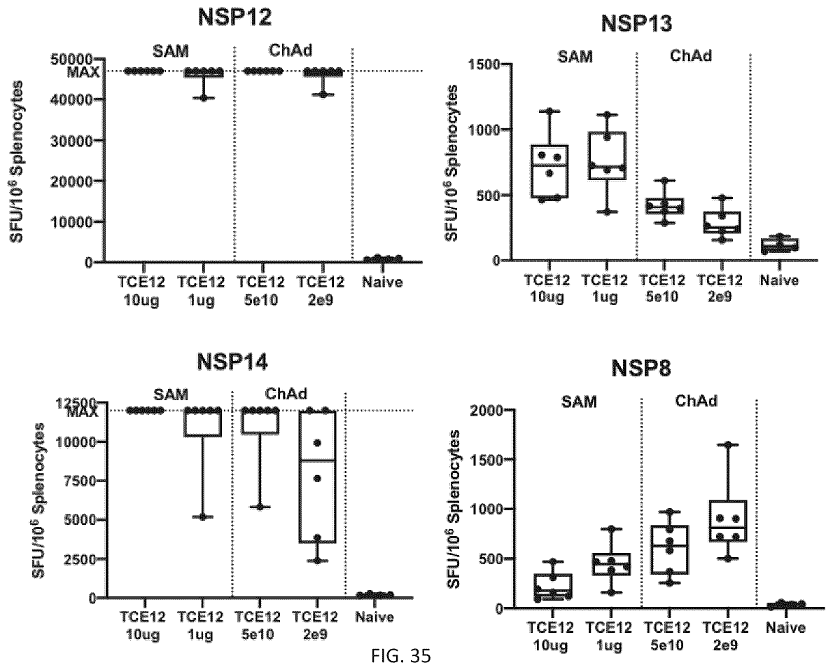

TCE12 was further assessed in a samRNA vector and compared to a ChAd-TCE12 vector. As shown in FIG. 35, strong immune responses against Nsp12 and 14 were observed for both SAM (left two series) and ChAd (right two series) vectors. Lower but detectable responses were also measured for both TCE vectors for Nsp13 and Nsp8.

### XIV.P. In Vivo Evaluation of RBD and TCE-Encoding SAM Vaccines

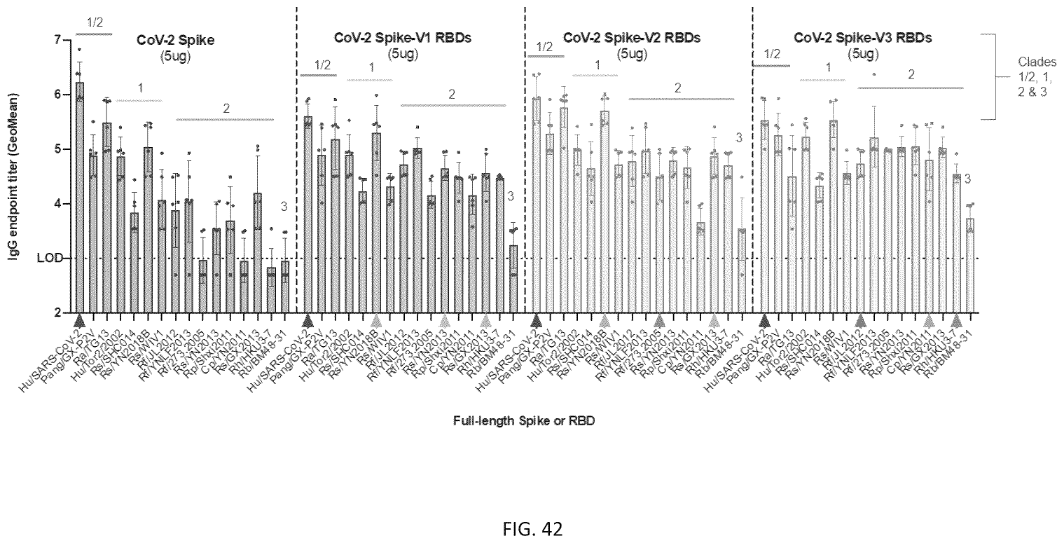

Single SAM vectors expressing Group 1 (“V1”), Group 2 (“V2”), or Group 3 (“V3”), antigens (SARS-COV2 Spike plus 3 sarbecovirus RBDs) were assessed following a single priming dose and compared to a SAM-SARS-COV2 vaccine only (“CoV-2 Spike”). As shown in FIG. 42 and quantified in Table 18, the vaccinations produced IgG immune responses to both vaccine component RBDs and diverse non-vaccine component RBDs. Groups 1, 2 and 3 vaccines showed comparable IgG immune responses. Notably, the breadth and overall magnitude to the non-vaccine component RBDs across multiple clades for the IgG immune response following administration of the Groups 1, 2 and 3 vaccines was increased relative the vaccine only encoding SARS-COV-2 Spike.

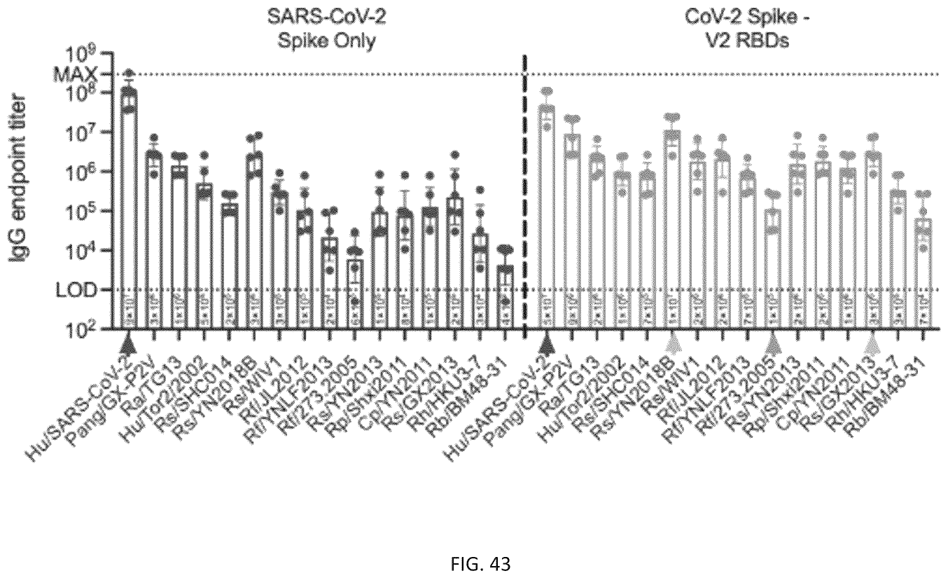

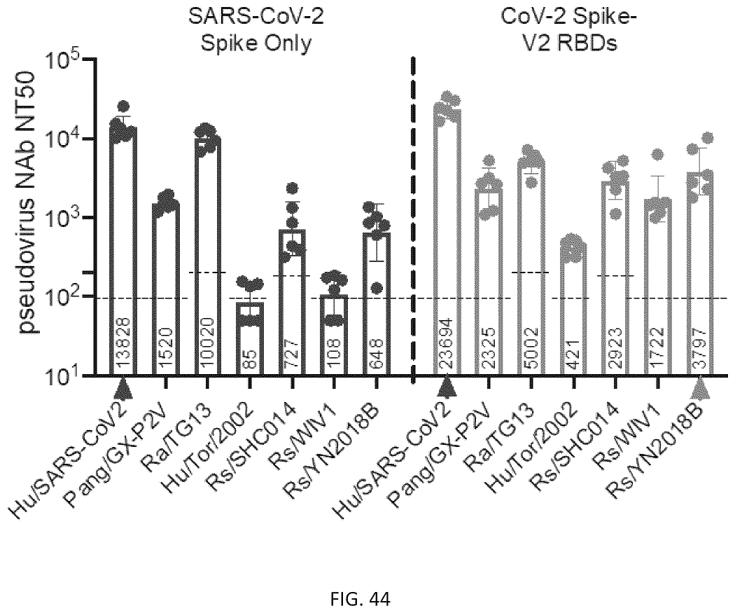

The Group 2 vaccine was further assessed following a homologous prime/boost strategy (boost at 4 weeks post prime dose and response assessed at week 8) and compared to a SAM-SARS-COV2 vaccine only. As shown in FIG. 43 and quantified in Table 19, the vaccinations produced IgG immune responses to both vaccine component RBDs and diverse non-vaccine component RBDs. As shown in FIG. 44 and quantified in Table 20, the vaccinations all produced neutralizing antibody titers to both vaccine component RBDs and diverse non-vaccine component RBDs. Again, the breadth and overall magnitude to the non-vaccine component RBDs across multiple clades for both the IgG immune response and neutralizing antibody titer following administration of the Group 2 vaccines was increased relative the vaccine only encoding SARS-COV-2 Spike.

These results demonstrate that vaccines encoding RBD domains were superior to the vaccine only encoding SARS-COV-2 Spike at generating immune responses against diverse non-vaccine component RBDs across multiple clades.

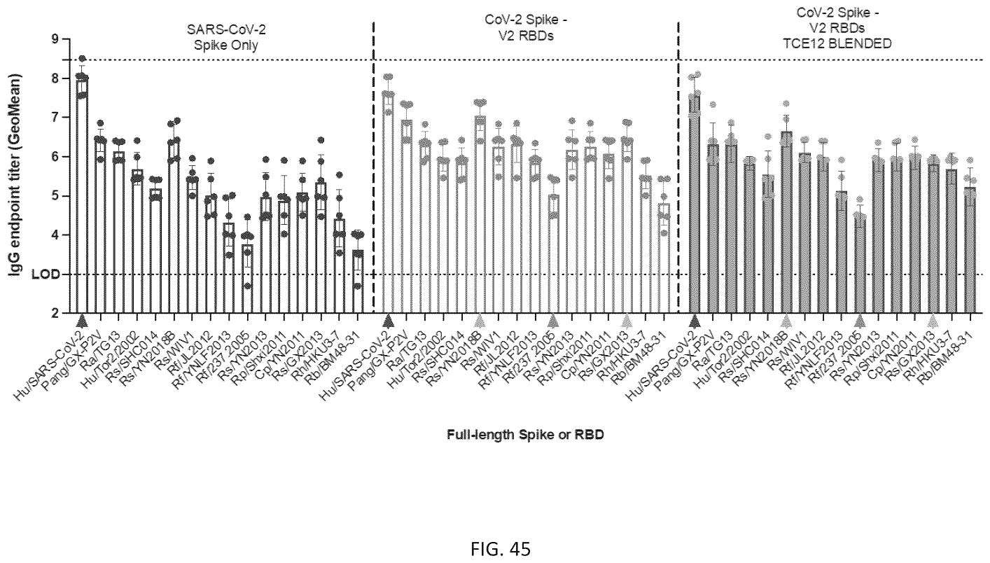

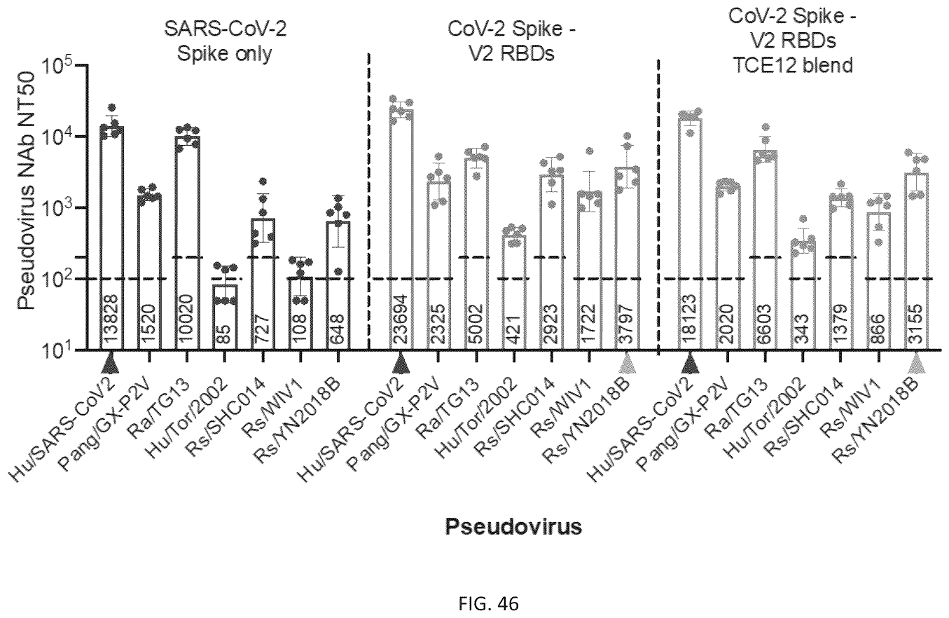

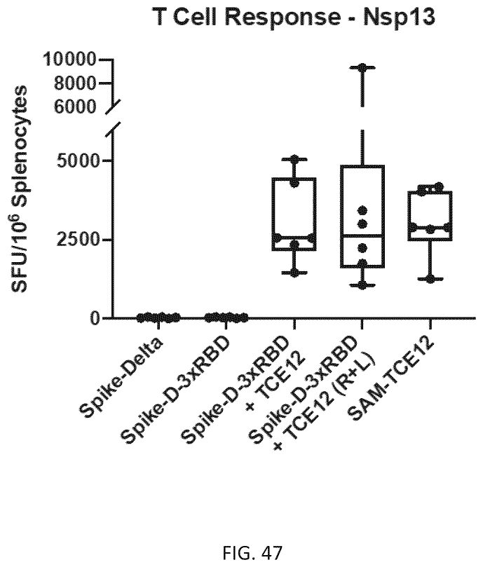

The Group 2 vaccine was additionally assessed administered in combination with a vector expressing the TCE12 epitope cassette (see Table 16A). The Group 2 and TCE12 vaccines were blended into a single composition and co-administered. As shown in FIG. 45 and quantified in Table 21, the vaccinations produced IgG immune responses to both vaccine component RBDs and diverse non-vaccine component RBDs. As shown in FIG. 46 and quantified in Table 22, the vaccinations all produced neutralizing antibody titers to both vaccine component RBDs and diverse non-vaccine component RBDs. Again, the breadth and overall magnitude to the non-vaccine component RBDs across multiple clades for both the IgG immune response and neutralizing antibody titer following administration of vaccines with Group 2 antigens, both alone or blended with TCE12, was increased relative the vaccine only encoding SARS-COV-2 Spike. Additionally, inclusion of TCE12 had minimal impact on the IgG response stimulated by the Group 2 vaccine. As shown in FIG. 47 and quantified in Table 23, the vaccinations with vectors encoding TCE12 all produced T cell responses to overlapping peptide pools for Nsp13. Similar results were observed for Nsp8, Nsp12, and Nsp14 (data not shown). Inclusion of RBD encoding vectors, administered either as a co-formulation or separately, had minimal impact T cell responses. These results demonstrate that combining vaccines encoding RBD domains and T cell epitopes stimulate their respective immune responses, while having minimal impact on each other.

TABLE 18
Anti-IgG Response to Spike/RBD

| | CoV-2 | GX-P2V | Ra/TG13 | Tor2 | RsSHC014 | YN2018B | WIV-1 | JL2012 | NLF2013 |
|---|---|---|---|---|---|---|---|---|---|
| SARS-CoV-2 | 1.7E+06 | 7.7E+04 | 3.1E+05 | 7.3E+04 | 7.0E+03 | 1.1E+05 | 1.2E+04 | 7.6E+03 | 1.1E+04 |
| SARS-CoV-2 | 4.0E+05 | 7.8E+04 | 1.6E+05 | 7.9E+04 | 1.7E+04 | 2.0E+05 | 2.1E+04 | 5.4E+04 | 1.1E+05 |
| Vaccine V1 | | | | | | | | | |
| SARS-CoV-2 | 8.4E+05 | 1.9E+05 | 5.9E+05 | 9.7E+04 | 4.4E+04 | 5.2E+05 | 5.4E+04 | 6.0E+04 | 9.4E+04 |
| Vaccine V2 | | | | | | | | | |
| SARS-CoV-2 | 3.4E+05 | 1.8E+05 | 3.3E+04 | 1.7E+05 | 2.2E+04 | 3.4E+05 | 3.7E+04 | 5.5E+04 | 1.7E+05 |
| Vaccine V3 | | | | | | | | | |

| | 273/2005 | YN2013 | Shx2011 | YN2011 | GX2013 | HKU3-7 | BM48-31 |
|---|---|---|---|---|---|---|---|
| SARS-CoV-2 | 9.3E+02 | 3.6E+03 | 5.1E+03 | 9.2E+02 | 1.6E+04 | 6.9E+02 | 9.1E+02 |
| SARS-CoV-2 | 1.5E+04 | 4.5E+04 | 3.0E+04 | 1.5E+04 | 3.7E+04 | 3.0E+04 | 1.8E+03 |
| Vaccine V1 | | | | | | | |
| SARS-CoV-2 | 3.1E+04 | 6.4E+04 | 4.6E+04 | 4.7E+03 | 7.3E+04 | 5.0E+04 | 3.4E+03 |
| Vaccine V2 | | | | | | | |
| SARS-CoV-2 | 9.6E+04 | 1.1E+05 | 1.1E+05 | 6.6E+04 | 1.1E+05 | 3.6E+04 | 5.5E+03 |
| Vaccine V3 | | | | | | | |

TABLE 19
8 week Anti-IgG Data

| | CoV-2 | GX-P2V | Ra/TG13 | Tor2 | RsSHC014 | YN2018B | WIV-1 | JL2012 | NF2013 |
|---|---|---|---|---|---|---|---|---|---|
| SARS-CoV-2 | 9.2E+07 | 2.6E+06 | 1.4E+06 | 5.0E+05 | 1.5E+05 | 2.5E+06 | 2.9E+05 | 1.1E+05 | 2.1E+04 |
| SARS-CoV-2 | 4.7E+07 | 8.9E+06 | 2.0E+06 | 9.7E+05 | 7.0E+05 | 1.1E+07 | 1.8E+06 | 2.1E+06 | 7.0E+05 |
| Vaccine V1 | | | | | | | | | |

| | 273/2005 | YN2013 | Shx2011 | YN2011 | GX2013 | HKU3-7 | BM48-31 |
|---|---|---|---|---|---|---|---|
| SARS-CoV-2 | 6.0E+03 | 9.6E+04 | 7.7E+04 | 1.3E+05 | 2.3E+05 | 2.7E+04 | 4.2E+03 |
| SARS-CoV-2 | 1.1E+05 | 1.5E+06 | 1.8E+06 | 1.2E+06 | 3.0E+06 | 3.4E+05 | 6.5E+04 |
| Vaccine V1 | | | | | | | |

TABLE 20
8 week PNA Data

| | CoV-2 | GX-P2V | Ra/TG13 | Tor2 | RsSHC014 | WIV-1 | YN2018B |
|---|---|---|---|---|---|---|---|
| SARS-CoV-2 | 13828 | 1520 | 10020 | 85 | 727 | 108 | 648 |
| SARS-CoV-2 | 23694 | 2335 | 5002 | 421 | 2923 | 1722 | 3797 |
| Vaccine V1 | | | | | | | |

TABLE 21
8 week IgG Data

| | CoV-2 | GX-P2V | Ra/TG13 | Tor2 | RsSHC014 | YN2018B | WIV-1 | JL2012 | NLF2013 |
|---|---|---|---|---|---|---|---|---|---|
| SARS-CoV-2 | 9.2E+07 | 2.6E+06 | 1.4E+06 | 5.0E+05 | 1.5E+03 | 2.3E+06 | 2.9E+05 | 1.1E+05 | 2.1E+04 |
| SARS-CoV-2 | 4.7E+07 | 8.9E+06 | 2.0E+06 | 9.7E+05 | 7.0E+05 | 1.1E+07 | 1.8E+06 | 2.1E+06 | 7.0E+05 |
| Vaccine V2 | | | | | | | | | |
| SARS-CoV-2 | 3.8E+07 | 2.1E+06 | 2.1E+06 | 6.8E+05 | 3.6E+05 | 4.5E+06 | 1.3E+06 | 1.0E+06 | 1.4E+05 |
| Vaccine V2 + | | | | | | | | | |
| TCE12 | | | | | | | | | |

| | 273/2005 | YN2013 | Shx2011 | YN2011 | GX2013 | HKU3-7 | BM48-31 |
|---|---|---|---|---|---|---|---|
| SARS-CoV-2 | 6.0E+03 | 9.6E+04 | 7.7E+04 | 1.3E+05 | 2.3E+05 | 2.7E+04 | 4.2E+03 |
| SARS-CoV-2 | 1.1E+05 | 1.5E+06 | 1.8E+06 | 1.2E+06 | 3.0E+06 | 3.4E+05 | 6.5E+04 |
| Vaccine V2 | | | | | | | |
| SARS-CoV-2 | 3.0E+04 | 8.0E+05 | 9.8E+05 | 9.2E+05 | 6.8E+05 | 4.9E+05 | 1.7E+05 |
| Vaccine V2 + | | | | | | | |
| TCE12 | | | | | | | |

TABLE 22
8 week PNA data

| | CoV-2 | GX-P2V | Ra/TG13 | Tor2 | RsSHC014 | WIV-1 | YN2018B |
|---|---|---|---|---|---|---|---|
| SARS-CoV-2 | 13828 | 1520 | 10020 | 85 | 727 | 108 | 648 |
| SARS-CoV-2 | 23694 | 2325 | 5002 | 421 | 2923 | 1722 | 3797 |
| V2 RBDs | | | | | | | |
| SARS-CoV-2 | 18123 | 2020 | 6603 | 343 | 1379 | 866 | 3155 |
| V2 RBDs + | | | | | | | |
| TCE12 | | | | | | | |

TABLE 23
ELISpot T-cell responses to Nsp13

| | | | Spike-D- | Spike-D- | |
|---|---|---|---|---|---|
| | Spike- | Spike-D- | 3xRBD + | 3xRBD + TCE12 | SAM- |
| | Delta | 3xRBD | TCE12 | (R + L) | TCE12 |
| | 27.2 | 28.0 | 4306.4 | 1066.9 | 2884.6 |
| | 51.0 | 33.1 | 5041.2 | 3004.6 | 1266.5 |
| | 0 | 28.2 | 2563.4 | 1750.7 | 2899.1 |
| | 21.5 | 56.6 | 1456.9 | 2243.6 | 4021.1 |
| | 60.0 | 7.6 | 2344.9 | 3428.7 | 2829.3 |
| | 38.4 | 52.2 | 2561.5 | 9300.0 | 4184.3 |
| Mean | 33.0 | 34.3 | 3045.7 | 3465.8 | 3014.2 |

### Additional Sequences

The sequences referred to below in Tables A-F refer to sequences found in International Application publication number WO2021236854A1 and U.S. Provisional Application No. 63/251,441, each of which is hereby incorporated by reference for all purposes.

### Table A

Refer to SEQ ID NOS. 130-8195 in the Sequence Listing found in International Application publication number WO2021236854A1 and U.S. Provisional Application No. 63/251,441, each of which is hereby incorporated by reference for all purposes. Presented is each candidate MHC Class I epitope encoded by SARS-COV-2 that was predicted to associate with a given HLA allele with an EDGE score >0.001. Each entry includes the candidate epitope sequence and cognate HLA alleles with a predicted EDGE score greater than 0.001, with each cognate pairing ranked as H (EDGE score >0.1), M (EDGE score between 0.01 and 0.1), and L (EDGE score <0.01). For example, the candidate epitope MESLVPGF (SEQ ID NO: 127) is predicted to pair with HLA-B*18:01, HLA-B*37:01, and HLA-B*07:05 with EDGE scores 0.019, 0.032, and 0.008, respectively. Accordingly, the entry for SEQ ID NO: 130 is “MESLVPGF: B18:01M; B37:01M; B07:05L.”

### Table B

Refer to SEQ ID NOS. 8196-26740 in the Sequence Listing found in International Application publication number WO2021236854A1 and U.S. Provisional Application No. 63/251,441, each of which is hereby incorporated by reference for all purposes. Presented is each candidate MHC Class II epitope encoded by SARS-COV-2 that was predicted to associate with a given HLA allele with an EDGE score >0.001. Each entry includes the candidate epitope sequence and cognate HLA alleles with a predicted EDGE score greater than 0.001, with each cognate pairing ranked as H (EDGE score >0.1), M (EDGE score between 0.01 and 0.1), and L (EDGE score <0.01). For example, the candidate epitope VELVAELEGI (SEQ ID NO: 128) is predicted to pair with HLA-DQA1*03:02-B1*03:03, HLA-DRB1*11:02, HLA-DQA1*05:05-B1*03:19, and HLA-DPA1*01:03-B1*104:01 with EDGE scores 0.003145, 0.00328, 0.041097, and 0.011613, respectively. Accordingly, the entry for SEQ ID NO: 8219 is “VELVAELEGI: DQA1*03:02-B1*03:03L; DRB1*11:02L; DQA1*05:05-B1*03:19M; DPA1*01:03-B1*104:01M.” Only HLA-DQ and HLA-DP are referred to by their alpha and beta chains. HLA-DR is referred to only by its beta chain as the alpha chain is generally invariable in the human population, with HLA-DR peptide contact regions particularly invariant.

### Table C

Refer to SEQ ID NOS. 26741-27179 in the Sequence Listing found in International Application publication number WO2021236854A1 and U.S. Provisional Application No. 63/251,441, each of which is hereby incorporated by reference for all purposes. Presented are additional MHC Class I epitopes, other than those from the Spike protein, encoded within the optimized cassette that were predicted to associate with a given HLA allele with an EDGE score >0.001. The additional epitopes were determined by calculating population coverage criteria P with all initial epitopes provided by the SARS-COV-2 Spike protein (SEQ ID NO:59) split into S1 and S2 and applying the optimization algorithms described herein.

### Table D

Refer to SEQ ID NOS. 27180-27495 in the Sequence Listing found in International Application publication number WO2021236854A1 and U.S. Provisional Application No. 63/251,441, each of which is hereby incorporated by reference for all purposes, for SARS-COV-2 Spike overlapping peptide pools. Each entry includes the stimulatory peptide, SARS-COV-2 protein source, peptide subpool information, and Table. For example, the stimulatory peptide MFVFLVLLPLVSSQC (SEQ ID NO: 27180) is derived from SARS-COV-2 Spike protein (Wuhan D614G variant), included in subpool S_Wu_1_2, and found in Table D. Accordingly, the entry for SEQ ID NO. 27180 is “MFVFLVLLPLVSSQC(SEQ ID NO:27180): Spike Wuhan D614G; S_Wu_1 2; Table D”.

### Table E

Refer to SEQ ID NOS. 27496-27603 in the Sequence Listing found in International Application publication number WO2021236854A1 and U.S. Provisional Application No. 63/251,441, each of which is hereby incorporated by reference for all purposes, for TCE5-encoded overlapping peptide pools. Each entry includes the stimulatory peptide, SARS-COV-2 protein source, peptide subpool information, and Table. For example, the stimulatory peptide LLWPVTLACFVLAAV (SEQ ID NO: 27496) is derived from SARS-COV-2 Membrane protein, included in subpool OLP_Mem, and found in Table E. Accordingly, the entry for SEQ ID NO. 27496 is “LLWPVTLACFVLAAV(SEQ ID NO:27496): Membrane; OLP_Mem; Table E”

### Table F

Refer to SEQ ID NOS. 27604-27939 in the Sequence Listing found in International Application publication number WO2021236854A1 and U.S. Provisional Application No. 63/251,441, each of which is hereby incorporated by reference for all purposes, for TCE5-encoded minimal epitope peptide pools. Each entry includes the stimulatory peptide, SARS-CoV-2 protein source, peptide subpool information, and Table. For example, the stimulatory peptide ALSKGVHFV (SEQ ID NO: 27604) is derived from SARS-COV-2 ORF3a protein (frame 52-85), included in subpool Min_validated, and found in Table F. Accordingly, the entry for SEQ ID NO. 27604 is “ALSKGVHFV(SEQ ID NO:27604): ORF3a 52-85; Min_validated; Table F”.

Certain additional sequences for vectors, cassettes, and antibodies referred to herein are described below and referred to by SEQ ID NO.

Tremelimumab VL (SEQ ID NO: 16)
Tremelimumab VH (SEQ ID NO: 17)
Tremelimumab VH CDR1 (SEQ ID NO: 18)
Tremelimumab VH CDR2 (SEQ ID NO: 19)
Tremelimumab VH CDR3 (SEQ ID NO: 20)
Tremelimumab VL CDR1 (SEQ ID NO: 21)
Tremelimumab VL CDR2 (SEQ ID NO: 22)
Tremelimumab VL CDR3 (SEQ ID NO: 23)
Durvalumab (MEDI4736) VL (SEQ ID NO: 24)
MEDI4736 VH (SEQ ID NO: 25)
MEDI4736 VH CDR1 (SEQ ID NO: 26)
MEDI4736 VH CDR2 (SEQ ID NO: 27)
MEDI4736 VH CDR3 (SEQ ID NO: 28)
MEDI4736 VL CDR1 (SEQ ID NO: 29)
MEDI4736 VL CDR2 (SEQ ID NO: 30)
MEDI4736 VL CDR3 (SEQ ID NO: 31)
UbA76-25merPDTT nucleotide (SEQ ID NO: 32)
UbA76-25merPDTT polypeptide (SEQ ID NO: 33)
MAG-25merPDTT nucleotide (SEQ ID NO: 34)
MAG-25merPDTT polypeptide (SEQ ID NO: 35)
Ub7625merPDTT_NoSFL nucleotide (SEQ ID NO: 36)
Ub7625merPDTT_NoSFL polypeptide (SEQ ID NO: 37)
ChAdV68.5WTnt.MAG25mer (SEQ ID NO: 2); AC_000011.1 with E1 (nt 577 to 3403) and E3 (nt 27,125-31,825)
sequences deleted; corresponding ATCC VR-594 nucleotides substituted at five positions; model neo-
antigen cassette under the control of the CMV promoter/enhancer inserted in place of deleted E1; SV40
polyA 3′ of cassette
Venezuelan equine encephalitis virus [VEE (SEQ ID NO: 3) GenBank: L01442.2
VEE-MAG25mer (SEQ ID NO: 4); contains MAG-25merPDTT nucleotide (bases 30-1755)
Venezuelan equine encephalitis virus strain TC-83 [TC-83(SEQ ID NO: 5) GenBank: L01443.1
ATAGGCGGCGCATGAGAGAAGCCCAGACCAATTACCTACCCAAAATGGAGAAAGTTCACGTTGAC
ATCGAGGAAGACAGCCCATTCCTCAGAGCTTTGCAGCGGAGCTTCCCGCAGTTTGAGGTAGAAGCC
AAGCAGGTCACTGATAATGACCATGCTAATGCCAGAGCGTTTTCGCATCTGGCTTCAAAACTGATC
GAAACGGAGGTGGACCCATCCGACACGATCCTTGACATTGGAAGTGCGCCCGCCCGCAGAATGTA
TTCTAAGCACAAGTATCATTGTATCTGTCCGATGAGATGTGCGGAAGATCCGGACAGATTGTATAA
GTATGCAACTAAGCTGAAGAAAAACTGTAAGGAAATAACTGATAAGGAATTGGACAAGAAAATG
AAGGAGCTCGCCGCCGTCATGAGCGACCCTGACCTGGAAACTGAGACTATGTGCCTCCACGACGA
CGAGTCGTGTCGCTACGAAGGGCAAGTCGCTGTTTACCAGGATGTATACGCGGTTGACGGACCGAC
AAGTCTCTATCACCAAGCCAATAAGGGAGTTAGAGTCGCCTACTGGATAGGCTTTGACACCACCCC
TTTTATGTTTAAGAACTTGGCTGGAGCATATCCATCATACTCTACCAACTGGGCCGACGAAACCGT
GTTAACGGCTCGTAACATAGGCCTATGCAGCTCTGACGTTATGGAGCGGTCACGTAGAGGGATGTC
CATTCTTAGAAAGAAGTATTTGAAACCATCCAACAATGTTCTATTCTCTGTTGGCTCGACCATCTAC
CACGAGAAGAGGGACTTACTGAGGAGCTGGCACCTGCCGTCTGTATTTCACTTACGTGGCAAGCAA
AATTACACATGTCGGTGTGAGACTATAGTTAGTTGCGACGGGTACGTCGTTAAAAGAATAGCTATC
AGTCCAGGCCTGTATGGGAAGCCTTCAGGCTATGCTGCTACGATGCACCGCGAGGGATTCTTGTGC
TGCAAAGTGACAGACACATTGAACGGGGAGAGGGTCTCTTTTCCCGTGTGCACGTATGTGCCAGCT
ACATTGTGTGACCAAATGACTGGCATACTGGCAACAGATGTCAGTGCGGACGACGCGCAAAAACT
GCTGGTTGGGCTCAACCAGCGTATAGTCGTCAACGGTCGCACCCAGAGAAACACCAATACCATGA
AAAATTACCTTTTGCCCGTAGTGGCCCAGGCATTTGCTAGGTGGGCAAAGGAATATAAGGAAGATC
AAGAAGATGAAAGGCCACTAGGACTACGAGATAGACAGTTAGTCATGGGGTGTTGTTGGGCTTTT
AGAAGGCACAAGATAACATCTATTTATAAGCGCCCGGATACCCAAACCATCATCAAAGTGAACAG
CGATTTCCACTCATTCGTGCTGCCCAGGATAGGCAGTAACACATTGGAGATCGGGCTGAGAACAAG
AATCAGGAAAATGTTAGAGGAGCACAAGGAGCCGTCACCTCTCATTACCGCCGAGGACGTACAAG
AAGCTAAGTGCGCAGCCGATGAGGCTAAGGAGGTGCGTGAAGCCGAGGAGTTGCGCGCAGCTCTA
CCACCTTTGGCAGCTGATGTTGAGGAGCCCACTCTGGAAGCCGATGTCGACTTGATGTTACAAGAG
GCTGGGGCCGGCTCAGTGGAGACACCTCGTGGCTTGATAAAGGTTACCAGCTACGATGGCGAGGA
CAAGATCGGCTCTTACGCTGTGCTTTCTCCGCAGGCTGTACTCAAGAGTGAAAAATTATCTTGCATC
CACCCTCTCGCTGAACAAGTCATAGTGATAACACACTCTGGCCGAAAAGGGCGTTATGCCGTGGAA
CCATACCATGGTAAAGTAGTGGTGCCAGAGGGACATGCAATACCCGTCCAGGACTTTCAAGCTCTG
AGTGAAAGTGCCACCATTGTGTACAACGAACGTGAGTTCGTAAACAGGTACCTGCACCATATTGCC
ACACATGGAGGAGCGCTGAACACTGATGAAGAATATTACAAAACTGTCAAGCCCAGCGAGCACGA
CGGCGAATACCTGTACGACATCGACAGGAAACAGTGCGTCAAGAAAGAACTAGTCACTGGGCTAG
GGCTCACAGGCGAGCTGGTGGATCCTCCCTTCCATGAATTCGCCTACGAGAGTCTGAGAACACGAC
CAGCCGCTCCTTACCAAGTACCAACCATAGGGGTGTATGGCGTGCCAGGATCAGGCAAGTCTGGC
ATCATTAAAAGCGCAGTCACCAAAAAAGATCTAGTGGTGAGCGCCAAGAAAGAAAACTGTGCAGA
AATTATAAGGGACGTCAAGAAAATGAAAGGGCTGGACGTCAATGCCAGAACTGTGGACTCAGTGC
TCTTGAATGGATGCAAACACCCCGTAGAGACCCTGTATATTGACGAAGCTTTTGCTTGTCATGCAG
GTACTCTCAGAGCGCTCATAGCCATTATAAGACCTAAAAAGGCAGTGCTCTGCGGGGATCCCAAAC
AGTGCGGTTTTTTTAACATGATGTGCCTGAAAGTGCATTTTAACCACGAGATTTGCACACAAGTCTT
CCACAAAAGCATCTCTCGCCGTTGCACTAAATCTGTGACTTCGGTCGTCTCAACCTTGTTTTACGAC
AAAAAAATGAGAACGACGAATCCGAAAGAGACTAAGATTGTGATTGACACTACCGGCAGTACCAA
ACCTAAGCAGGACGATCTCATTCTCACTTGTTTCAGAGGGTGGGTGAAGCAGTTGCAAATAGATTA
CAAAGGCAACGAAATAATGACGGCAGCTGCCTCTCAAGGGCTGACCCGTAAAGGTGTGTATGCCG
TTCGGTACAAGGTGAATGAAAATCCTCTGTACGCACCCACCTCAGAACATGTGAACGTCCTACTGA
CCCGCACGGAGGACCGCATCGTGTGGAAAACACTAGCCGGCGACCCATGGATAAAAACACTGACT
GCCAAGTACCCTGGGAATTTCACTGCCACGATAGAGGAGTGGCAAGCAGAGCATGATGCCATCAT
GAGGCACATCTTGGAGAGACCGGACCCTACCGACGTCTTCCAGAATAAGGCAAACGTGTGTTGGG
CCAAGGCTTTAGTGCCGGTGCTGAAGACCGCTGGCATAGACATGACCACTGAACAATGGAACACT
GTGGATTATTTTGAAACGGACAAAGCTCACTCAGCAGAGATAGTATTGAACCAACTATGCGTGAG
GTTCTTTGGACTCGATCTGGACTCCGGTCTATTTTCTGCACCCACTGTTCCGTTATCCATTAGGAAT
AATCACTGGGATAACTCCCCGTCGCCTAACATGTACGGGCTGAATAAAGAAGTGGTCCGTCAGCTC
TCTCGCAGGTACCCACAACTGCCTCGGGCAGTTGCCACTGGAAGAGTCTATGACATGAACACTGGT
ACACTGCGCAATTATGATCCGCGCATAAACCTAGTACCTGTAAACAGAAGACTGCCTCATGCTTTA
GTCCTCCACCATAATGAACACCCACAGAGTGACTTTTCTTCATTCGTCAGCAAATTGAAGGGCAGA
ACTGTCCTGGTGGTCGGGGAAAAGTTGTCCGTCCCAGGCAAAATGGTTGACTGGTTGTCAGACCGG
CCTGAGGCTACCTTCAGAGCTCGGCTGGATTTAGGCATCCCAGGTGATGTGCCCAAATATGACATA
ATATTTGTTAATGTGAGGACCCCATATAAATACCATCACTATCAGCAGTGTGAAGACCATGCCATT
AAGCTTAGCATGTTGACCAAGAAAGCTTGTCTGCATCTGAATCCCGGCGGAACCTGTGTCAGCATA
GGTTATGGTTACGCTGACAGGGCCAGCGAAAGCATCATTGGTGCTATAGCGCGGCAGTTCAAGTTT
TCCCGGGTATGCAAACCGAAATCCTCACTTGAAGAGACGGAAGTTCTGTTTGTATTCATTGGGTAC
GATCGCAAGGCCCGTACGCACAATCCTTACAAGCTTTCATCAACCTTGACCAACATTTATACAGGT
TCCAGACTCCACGAAGCCGGATGTGCACCCTCATATCATGTGGTGCGAGGGGATATTGCCACGGCC
ACCGAAGGAGTGATTATAAATGCTGCTAACAGCAAAGGACAACCTGGCGGAGGGGTGTGCGGAGC
GCTGTATAAGAAATTCCCGGAAAGCTTCGATTTACAGCCGATCGAAGTAGGAAAAGCGCGACTGG
TCAAAGGTGCAGCTAAACATATCATTCATGCCGTAGGACCAAACTTCAACAAAGTTTCGGAGGTTG
AAGGTGACAAACAGTTGGCAGAGGCTTATGAGTCCATCGCTAAGATTGTCAACGATAACAATTAC
AAGTCAGTAGCGATTCCACTGTTGTCCACCGGCATCTTTTCCGGGAACAAAGATCGACTAACCCAA
TCATTGAACCATTTGCTGACAGCTTTAGACACCACTGATGCAGATGTAGCCATATACTGCAGGGAC
AAGAAATGGGAAATGACTCTCAAGGAAGCAGTGGCTAGGAGAGAAGCAGTGGAGGAGATATGCA
TATCCGACGACTCTTCAGTGACAGAACCTGATGCAGAGCTGGTGAGGGTGCATCCGAAGAGTTCTT
TGGCTGGAAGGAAGGGCTACAGCACAAGCGATGGCAAAACTTTCTCATATTTGGAAGGGACCAAG
TTTCACCAGGCGGCCAAGGATATAGCAGAAATTAATGCCATGTGGCCCGTTGCAACGGAGGCCAA
TGAGCAGGTATGCATGTATATCCTCGGAGAAAGCATGAGCAGTATTAGGTCGAAATGCCCCGTCG
AAGAGTCGGAAGCCTCCACACCACCTAGCACGCTGCCTTGCTTGTGCATCCATGCCATGACTCCAG
AAAGAGTACAGCGCCTAAAAGCCTCACGTCCAGAACAAATTACTGTGTGCTCATCCTTTCCATTGC
CGAAGTATAGAATCACTGGTGTGCAGAAGATCCAATGCTCCCAGCCTATATTGTTCTCACCGAAAG
TGCCTGCGTATATTCATCCAAGGAAGTATCTCGTGGAAACACCACCGGTAGACGAGACTCCGGAGC
CATCGGCAGAGAACCAATCCACAGAGGGGACACCTGAACAACCACCACTTATAACCGAGGATGAG
ACCAGGACTAGAACGCCTGAGCCGATCATCATCGAAGAGGAAGAAGAGGATAGCATAAGTTTGCT
GTCAGATGGCCCGACCCACCAGGTGCTGCAAGTCGAGGCAGACATTCACGGGCCGCCCTCTGTATC
TAGCTCATCCTGGTCCATTCCTCATGCATCCGACTTTGATGTGGACAGTTTATCCATACTTGACACC
CTGGAGGGAGCTAGCGTGACCAGCGGGGCAACGTCAGCCGAGACTAACTCTTACTTCGCAAAGAG
TATGGAGTTTCTGGCGCGACCGGTGCCTGCGCCTCGAACAGTATTCAGGAACCCTCCACATCCCGC
TCCGCGCACAAGAACACCGTCACTTGCACCCAGCAGGGCCTGCTCGAGAACCAGCCTAGTTTCCAC
CCCGCCAGGCGTGAATAGGGTGATCACTAGAGAGGAGCTCGAGGCGCTTACCCCGTCACGCACTC
CTAGCAGGTCGGTCTCGAGAACCAGCCTGGTCTCCAACCCGCCAGGCGTAAATAGGGTGATTACA
AGAGAGGAGTTTGAGGCGTTCGTAGCACAACAACAATGACGGTTTGATGCGGGTGCATACATCTTT
TCCTCCGACACCGGTCAAGGGCATTTACAACAAAAATCAGTAAGGCAAACGGTGCTATCCGAAGT
GGTGTTGGAGAGGACCGAATTGGAGATTTCGTATGCCCCGCGCCTCGACCAAGAAAAAGAAGAAT
TACTACGCAAGAAATTACAGTTAAATCCCACACCTGCTAACAGAAGCAGATACCAGTCCAGGAAG
GTGGAGAACATGAAAGCCATAACAGCTAGACGTATTCTGCAAGGCCTAGGGCATTATTTGAAGGC
AGAAGGAAAAGTGGAGTGCTACCGAACCCTGCATCCTGTTCCTTTGTATTCATCTAGTGTGAACCG
TGCCTTTTCAAGCCCCAAGGTCGCAGTGGAAGCCTGTAACGCCATGTTGAAAGAGAACTTTCCGAC
TGTGGCTTCTTACTGTATTATTCCAGAGTACGATGCCTATTTGGACATGGTTGACGGAGCTTCATGC
TGCTTAGACACTGCCAGTTTTTGCCCTGCAAAGCTGCGCAGCTTTCCAAAGAAACACTCCTATTTGG
AACCCACAATACGATCGGCAGTGCCTTCAGCGATCCAGAACACGCTCCAGAACGTCCTGGCAGCT
GCCACAAAAAGAAATTGCAATGTCACGCAAATGAGAGAATTGCCCGTATTGGATTCGGCGGCCTTT
AATGTGGAATGCTTCAAGAAATATGCGTGTAATAATGAATATTGGGAAACGTTTAAAGAAAACCC
CATCAGGCTTACTGAAGAAAACGTGGTAAATTACATTACCAAATTAAAAGGACCAAAAGCTGCTG
CTCTTTTTGCGAAGACACATAATTTGAATATGTTGCAGGACATACCAATGGACAGGTTTGTAATGG
ACTTAAAGAGAGACGTGAAAGTGACTCCAGGAACAAAACATACTGAAGAACGGCCCAAGGTACA
GGTGATCCAGGCTGCCGATCCGCTAGCAACAGCGTATCTGTGCGGAATCCACCGAGAGCTGGTTAG
GAGATTAAATGCGGTCCTGCTTCCGAACATTCATACACTGTTTGATATGTCGGCTGAAGACTTTGA
CGCTATTATAGCCGAGCACTTCCAGCCTGGGGATTGTGTTCTGGAAACTGACATCGCGTCGTTTGA
TAAAAGTGAGGACGACGCCATGGCTCTGACCGCGTTAATGATTCTGGAAGACTTAGGTGTGGACG
CAGAGCTGTTGACGCTGATTGAGGCGGCTTTCGGCGAAATTTCATCAATACATTTGCCCACTAAAA
CTAAATTTAAATTCGGAGCCATGATGAAATCTGGAATGTTCCTCACACTGTTTGTGAACACAGTCA
TTAACATTGTAATCGCAAGCAGAGTGTTGAGAGAACGGCTAACCGGATCACCATGTGCAGCATTCA
TTGGAGATGACAATATCGTGAAAGGAGTCAAATCGGACAAATTAATGGCAGACAGGTGCGCCACC
TGGTTGAATATGGAAGTCAAGATTATAGATGCTGTGGTGGGCGAGAAAGCGCCTTATTTCTGTGGA
GGGTTTATTTTGTGTGACTCCGTGACCGGCACAGCGTGCCGTGTGGCAGACCCCCTAAAAAGGCTG
TTTAAGCTTGGCAAACCTCTGGCAGCAGACGATGAACATGATGATGACAGGAGAAGGGCATTGCA
TGAAGAGTCAACACGCTGGAACCGAGTGGGTATTCTTTCAGAGCTGTGCAAGGCAGTAGAATCAA
GGTATGAAACCGTAGGAACTTCCATCATAGTTATGGCCATGACTACTCTAGCTAGCAGTGTTAAAT
CATTCAGCTACCTGAGAGGGGCCCCTATAACTCTCTACGGCTAACCTGAATGGACTACGACATAGT
CTAGTCCGCCAAGATGTTCCCGTTCCAGCCAATGTATCCGATGCAGCCAATGCCCTATCGCAACCC
GTTCGCGGCCCCGCGCAGGCCCTGGTTCCCCAGAACCGACCCTTTTCTGGCGATGCAGGTGCAGGA
ATTAACCCGCTCGATGGCTAACCTGACGTTCAAGCAACGCCGGGACGCGCCACCTGAGGGGCCAT
CCGCTAAGAAACCGAAGAAGGAGGCCTCGCAAAAACAGAAAGGGGGAGGCCAAGGGAAGAAGA
AGAAGAACCAAGGGAAGAAGAAGGCTAAGACAGGGCCGCCTAATCCGAAGGCACAGAATGGAAA
CAAGAAGAAGACCAACAAGAAACCAGGCAAGAGACAGCGCATGGTCATGAAATTGGAATCTGAC
AAGACGTTCCCAATCATGTTGGAAGGGAAGATAAACGGCTACGCTTGTGTGGTCGGAGGGAAGTT
ATTCAGGCCGATGCATGTGGAAGGCAAGATCGACAACGACGTTCTGGCCGCGCTTAAGACGAAGA
AAGCATCCAAATACGATCTTGAGTATGCAGATGTGCCACAGAACATGCGGGCCGATACATTCAAA
TACACCCATGAGAAACCCCAAGGCTATTACAGCTGGCATCATGGAGCAGTCCAATATGAAAATGG
GCGTTTCACGGTGCCGAAAGGAGTTGGGGCCAAGGGAGACAGCGGACGACCCATTCTGGATAACC
AGGGACGGGTGGTCGCTATTGTGCTGGGAGGTGTGAATGAAGGATCTAGGACAGCCCTTTCAGTC
GTCATGTGGAACGAGAAGGGAGTTACCGTGAAGTATACTCCGGAGAACTGCGAGCAATGGTCACT
AGTGACCACCATGTGTCTGCTCGCCAATGTGACGTTCCCATGTGCTCAACCACCAATTTGCTACGA
CAGAAAACCAGCAGAGACTTTGGCCATGCTCAGCGTTAACGTTGACAACCCGGGCTACGATGAGC
TGCTGGAAGCAGCTGTTAAGTGCCCCGGAAGGAAAAGGAGATCCACCGAGGAGCTGTTTAATGAG
TATAAGCTAACGCGCCCTTACATGGCCAGATGCATCAGATGTGCAGTTGGGAGCTGCCATAGTCCA
ATAGCAATCGAGGCAGTAAAGAGCGACGGGCACGACGGTTATGTTAGACTTCAGACTTCCTCGCA
GTATGGCCTGGATTCCTCCGGCAACTTAAAGGGCAGGACCATGCGGTATGACATGCACGGGACCA
TTAAAGAGATACCACTACATCAAGTGTCACTCTATACATCTCGCCCGTGTCACATTGTGGATGGGC
ACGGTTATTTCCTGCTTGCCAGGTGCCCGGCAGGGGACTCCATCACCATGGAATTTAAGAAAGATT
CCGTCAGACACTCCTGCTCGGTGCCGTATGAAGTGAAATTTAATCCTGTAGGCAGAGAACTCTATA
CTCATCCCCCAGAACACGGAGTAGAGCAAGCGTGCCAAGTCTACGCACATGATGCACAGAACAGA
GGAGCTTATGTCGAGATGCACCTCCCGGGCTCAGAAGTGGACAGCAGTTTGGTTTCCTTGAGCGGC
AGTTCAGTCACCGTGACACCTCCTGATGGGACTAGCGCCCTGGTGGAATGCGAGTGTGGCGGCACA
AAGATCTCCGAGACCATCAACAAGACAAAACAGTTCAGCCAGTGCACAAAGAAGGAGCAGTGCA
GAGCATATCGGCTGCAGAACGATAAGTGGGTGTATAATTCTGACAAACTGCCCAAAGCAGCGGGA
GCCACCTTAAAAGGAAAACTGCATGTCCCATTCTTGCTGGCAGACGGCAAATGCACCGTGCCTCTA
GCACCAGAACCTATGATAACCTTCGGTTTCAGATCAGTGTCACTGAAACTGCACCCTAAGAATCCC
ACATATCTAATCACCCGCCAACTTGCTGATGAGCCTCACTACACGCACGAGCTCATATCTGAACCA
GCTGTTAGGAATTTTACCGTCACCGAAAAAGGGTGGGAGTTTGTATGGGGAAACCACCCGCCGAA
AAGGTTTTGGGCACAGGAAACAGCACCCGGAAATCCACATGGGCTACCGCACGAGGTGATAACTC
ATTATTACCACAGATACCCTATGTCCACCATCCTGGGTTTGTCAATTTGTGCCGCCATTGCAACCGT
TTCCGTTGCAGCGTCTACCTGGCTGTTTTGCAGATCTAGAGTTGCGTGCCTAACTCCTTACCGGCTA
ACACCTAACGCTAGGATACCATTTTGTCTGGCTGTGCTTTGCTGCGCCCGCACTGCCCGGGCCGAG
ACCACCTGGGAGTCCTTGGATCACCTATGGAACAATAACCAACAGATGTTCTGGATTCAATTGCTG
ATCCCTCTGGCCGCCTTGATCGTAGTGACTCGCCTGCTCAGGTGCGTGTGCTGTGTCGTGCCTTTTT
TAGTCATGGCCGGCGCCGCAGGCGCCGGCGCCTACGAGCACGCGACCACGATGCCGAGCCAAGCG
GGAATCTCGTATAACACTATAGTCAACAGAGCAGGCTACGCACCACTCCCTATCAGCATAACACCA
ACAAAGATCAAGCTGATACCTACAGTGAACTTGGAGTACGTCACCTGCCACTACAAAACAGGAAT
GGATTCACCAGCCATCAAATGCTGCGGATCTCAGGAATGCACTCCAACTTACAGGCCTGATGAACA
GTGCAAAGTCTTCACAGGGGTTTACCCGTTCATGTGGGGTGGTGCATATTGCTTTTGCGACACTGA
GAACACCCAAGTCAGCAAGGCCTACGTAATGAAATCTGACGACTGCCTTGCGGATCATGCTGAAG
CATATAAAGCGCACACAGCCTCAGTGCAGGCGTTCCTCAACATCACAGTGGGAGAACACTCTATTG
TGACTACCGTGTATGTGAATGGAGAAACTCCTGTGAATTTCAATGGGGTCAAAATAACTGCAGGTC
CGCTTTCCACAGCTTGGACACCCTTTGATCGCAAAATCGTGCAGTATGCCGGGGAGATCTATAATT
ATGATTTTCCTGAGTATGGGGCAGGACAACCAGGAGCATTTGGAGATATACAATCCAGAACAGTCT
CAAGCTCTGATCTGTATGCCAATACCAACCTAGTGCTGCAGAGACCCAAAGCAGGAGCGATCCAC
GTGCCATACACTCAGGCACCTTCGGGTTTTGAGCAATGGAAGAAAGATAAAGCTCCATCATTGAAA
TTTACCGCCCCTTTCGGATGCGAAATATATACAAACCCCATTCGCGCCGAAAACTGTGCTGTAGGG
TCAATTCCATTAGCCTTTGACATTCCCGACGCCTTGTTCACCAGGGTGTCAGAAACACCGACACTTT
CAGCGGCCGAATGCACTCTTAACGAGTGCGTGTATTCTTCCGACTTTGGTGGGATCGCCACGGTCA
AGTACTCGGCCAGCAAGTCAGGCAAGTGCGCAGTCCATGTGCCATCAGGGACTGCTACCCTAAAA
GAAGCAGCAGTCGAGCTAACCGAGCAAGGGTCGGCGACTATCCATTTCTCGACCGCAAATATCCA
CCCGGAGTTCAGGCTCCAAATATGCACATCATATGTTACGTGCAAAGGTGATTGTCACCCCCCGAA
AGACCATATTGTGACACACCCTCAGTATCACGCCCAAACATTTACAGCCGCGGTGTCAAAAACCGC
GTGGACGTGGTTAACATCCCTGCTGGGAGGATCAGCCGTAATTATTATAATTGGCTTGGTGCTGGC
TACTATTGTGGCCATGTACGTGCTGACCAACCAGAAACATAATTGAATACAGCAGCAATTGGCAAG
CTGCTTACATAGAACTCGCGGCGATTGGCATGCCGCCTTAAAATTTTTATTTTATTTTTCTTTTCTTT
TCCGAATCGGATTTTGTTTTTAATATTTC
VEE Delivery Vector (SEQ ID NO: 6); VEE genome with nucleotides 7544-11175 deleted [alphavirus
structural proteins removed
ATGggcggcgcatgagagaagcccagaccaattacctacccaaaATGGagaaagttcacgttgacatcgaggaagacagccc
attcctcagagctttgcagcggagcttcccgcagtttgaggtagaagccaagcaggtcactgataatgaccatgctaatgccagagcgt
tttcgcatctggcttcaaaactgatcgaaacggaggtggacccatccgacacgatccttgacattggaagtgcgcccgcccgcagaat
gtattctaagcacaagtatcattgtatctgtccgatgagatgtgcggaagatccggacagattgtataagtatgcaactaagctgaagaaa
aactgtaaggaaataactgataaggaattggacaagaaaatgaaggagctcgccgccgtcatgagcgaccctgacctggaaactgag
actatgtgcctccacgacgacgagtcgtgtcgctacgaagggcaagtcgctgtttaccaggatgtatacgcggttgacggaccgacaa
gtctctatcaccaagccaataagggagttagagtcgcctactggataggctttgacaccaccccttttatgtttaagaacttggctggagc
atatccatcatactctaccaactgggccgacgaaaccgtgttaacggctcgtaacataggcctatgcagctctgacgttatggagcggtc
acgtagagggatgtccattcttagaaagaagtatttgaaaccatccaacaatgttctattctctgttggctcgaccatctaccacgagaaga
gggacttactgaggagctggcacctgccgtctgtatttcacttacgtggcaagcaaaattacacatgtcggtgtgagactatagttagttg
cgacgggtacgtcgttaaaagaatagctatcagtccaggcctgtatgggaagccttcaggctatgctgctacgatgcaccgcgaggga
ttcttgtgctgcaaagtgacagacacattgaacggggagagggtctcttttcccgtgtgcacgtatgtgccagctacattgtgtgaccaaa
tgactggcatactggcaacagatgtcagtgcggacgacgcgcaaaaactgctggttgggctcaaccagcgtatagtcgtcaacggtc
gcacccagagaaacaccaataccatgaaaaattaccttttgcccgtagtggcccaggcatttgctaggtgggcaaaggaatataagga
agatcaagaagatgaaaggccactaggactacgagatagacagttagtcatggggtgttgttgggcttttagaaggcacaagataacat
ctatttataagcgcccggatacccaaaccatcatcaaagtgaacagcgatttccactcattcgtgctgcccaggataggcagtaacacat
tggagatcgggctgagaacaagaatcaggaaaatgttagaggagcacaaggagccgtcacctctcattaccgccgaggacgtacaa
gaagctaagtgcgcagccgatgaggctaaggaggtgcgtgaagccgaggagttgcgcgcagctctaccacctttggcagctgatgtt
gaggagcccactctggaagccgatgtcgacttgatgttacaagaggctggggccggctcagtggagacacctcgtggcttgataaag
gttaccagctacgctggcgaggacaagatcggctcttacgctgtgctttctccgcaggctgtactcaagagtgaaaaattatcttgcatcc
accctctcgctgaacaagtcatagtgataacacactctggccgaaaagggcgttatgccgtggaaccataccatggtaaagtagtggtg
ccagagggacatgcaatacccgtccaggactttcaagctctgagtgaaagtgccaccattgtgtacaacgaacgtgagttcgtaaaca
ggtacctgcaccatattgccacacatggaggagcgctgaacactgatgaagaatattacaaaactgtcaagcccagcgagcacgacg
gcgaatacctgtacgacatcgacaggaaacagtgcgtcaagaaagaactagtcactgggctagggctcacaggcgagctggtggat
cctcccttccatgaattcgcctacgagagtctgagaacacgaccagccgctccttaccaagtaccaaccataggggtgtatggcgtgcc
aggatcaggcaagtctggcatcattaaaagcgcagtcaccaaaaaagatctagtggtgagcgccaagaaagaaaactgtgcagaaat
tataagggacgtcaagaaaatgaaagggctggacgtcaatgccagaactgtggactcagtgctcttgaatggatgcaaacaccccgta
gagaccctgtatattgacgaagcttttgcttgtcatgcaggtactctcagagcgctcatagccattataagacctaaaaaggcagtgctct
gcggggatcccaaacagtgcggtttttttaacatgatgtgcctgaaagtgcattttaaccacgagatttgcacacaagtcttccacaaaag
catctctcgccgttgcactaaatctgtgacttcggtcgtctcaaccttgttttacgacaaaaaaatgagaacgacgaatccgaaagagact
aagattgtgattgacactaccggcagtaccaaacctaagcaggacgatctcattctcacttgtttcagaggggggtgaagcagttgcaa
atagattacaaaggcaacgaaataatgacggcagctgcctctcaagggctgacccgtaaaggtgtgtatgccgttcggtacaaggtga
atgaaaatcctctgtacgcacccacctcagaacatgtgaacgtcctactgacccgcacggaggaccgcatcgtgtggaaaacactagc
cggcgacccatggataaaaacactgactgccaagtaccctgggaatttcactgccacgatagaggagtggcaagcagagcatgatgc
catcatgaggcacatcttggagagaccggaccctaccgacgtcttccagaataaggcaaacgtgtgttgggccaaggctttagtgccg
gtgctgaagaccgctggcatagacatgaccactgaacaatggaacactgtggattattttgaaacggacaaagctcactcagcagaga
tagtattgaaccaactatgcgtgaggttctttggactcgatctggactccggtctattttctgcacccactgttccgttatccattaggaataa
tcactgggataactccccgtcgcctaacatgtacgggctgaataaagaagtggtccgtcagctctctcgcaggtacccacaactgcctc
gggcagttgccactggaagagtctatgacatgaacactggtacactgcgcaattatgatccgcgcataaacctagtacctgtaaacaga
agactgcctcatgctttagtcctccaccataatgaacacccacagagtgacttttcttcattcgtcagcaaattgaagggcagaactgtcct
ggtggtcggggaaaagttgtccgtcccaggcaaaatggttgactggttgtcagaccggcctgaggctaccttcagagctcggctggat
ttaggcatcccaggtgatgtgcccaaatatgacataatatttgttaatgtgaggaccccatataaataccatcactatcagcagtgtgaaga
ccatgccattaagcttagcatgttgaccaagaaagcttgtctgcatctgaatcccggcggaacctgtgtcagcataggttatggttacgct
gacagggccagcgaaagcatcattggtgctatagcgcggcagttcaagttttcccgggtatgcaaaccgaaatcctcacttgaagaga
cggaagttctgtttgtattcattgggtacgatcgcaaggcccgtacgcacaatccttacaagctttcatcaaccttgaccaacatttataca
ggttccagactccacgaagccggatgtgcaccctcatatcatgtggtgcgaggggatattgccacggccaccgaaggagtgattataa
atgctgctaacagcaaaggacaacctggcggaggggtgtgcggagcgctgtataagaaattcccggaaagcttcgatttacagccga
tcgaagtaggaaaagcgcgactggtcaaaggtgcagctaaacatatcattcatgccgtaggaccaaacttcaacaaagtttcggaggtt
gaaggtgacaaacagttggcagaggcttatgagtccatcgctaagattgtcaacgataacaattacaagtcagtagcgattccactgttg
tccaccggcatcttttccgggaacaaagatcgactaacccaatcattgaaccatttgctgacagctttagacaccactgatgcagatgtag
ccatatactgcagggacaagaaatgggaaatgactctcaaggaagcagtggctaggagagaagcagtggaggagatatgcatatcc
gacgactcttcagtgacagaacctgatgcagagctggtgagggtgcatccgaagagttctttggctggaaggaagggctacagcaca
agcgatggcaaaactttctcatatttggaagggaccaagtttcaccaggcggccaaggatatagcagaaattaatgccatgtggcccgt
tgcaacggaggccaatgagcaggtatgcatgtatatcctcggagaaagcatgagcagtattaggtcgaaatgccccgtcgaagagtc
ggaagcctccacaccacctagcacgctgccttgcttgtgcatccatgccatgactccagaaagagtacagcgcctaaaagcctcacgt
ccagaacaaattactgtgtgctcatcctttccattgccgaagtatagaatcactggtgtgcagaagatccaatgctcccagcctatattgtt
ctcaccgaaagtgcctgcgtatattcatccaaggaagtatctcgtggaaacaccaccggtagacgagactccggagccatcggcaga
gaaccaatccacagaggggacacctgaacaaccaccacttataaccgaggatgagaccaggactagaacgcctgagccgatcatca
tcgaagaggaagaagaggatagcataagtttgctgtcagatggcccgacccaccaggtgctgcaagtcgaggcagacattcacggg
ccgccctctgtatctagctcatcctggtccattcctcatgcatccgactttgatgtggacagtttatccatacttgacaccctggagggagct
agcgtgaccagcggggcaacgtcagccgagactaactcttacttcgcaaagagtatggagtttctggcgcgaccggtgcctgcgcct
cgaacagtattcaggaaccctccacatcccgctccgcgcacaagaacaccgtcacttgcacccagcagggcctgctcgagaaccag
cctagtttccaccccgccaggcgtgaatagggtgatcactagagaggagctcgaggcgcttaccccgtcacgcactcctagcaggtc
ggtctcgagaaccagcctggtctccaacccgccaggcgtaaatagggtgattacaagagaggagtttgaggcgttcgtagcacaaca
acaatgacggtttgatgcgggtgcatacatcttttcctccgacaccggtcaagggcatttacaacaaaaatcagtaaggcaaacggtgct
atccgaagtggtgttggagaggaccgaattggagatttcgtatgccccgcgcctcgaccaagaaaaagaagaattactacgcaagaa
attacagttaaatcccacacctgctaacagaagcagataccagtccaggaaggtggagaacatgaaagccataacagctagacgtatt
ctgcaaggcctagggcattatttgaaggcagaaggaaaagtggagtgctaccgaaccctgcatcctgttcctttgtattcatctagtgtga
accgtgccttttcaagccccaaggtcgcagtggaagcctgtaacgccatgttgaaagagaactttccgactgtggcttcttactgtattatt
ccagagtacgatgcctatttggacatggttgacggagcttcatgctgcttagacactgccagtttttgccctgcaaagctgcgcagctttc
caaagaaacactcctatttggaacccacaatacgatcggcagtgccttcagcgatccagaacacgctccagaacgtcctggcagctgc
cacaaaaagaaattgcaatgtcacgcaaatgagagaattgcccgtattggattcggcggcctttaatgtggaatgcttcaagaaatatgc
gtgtaataatgaatattgggaaacgtttaaagaaaaccccatcaggcttactgaagaaaacgtggtaaattacattaccaaattaaaagga
ccaaaagctgctgctctttttgcgaagacacataatttgaatatgttgcaggacataccaatggacaggtttgtaatggacttaaagagag
acgtgaaagtgactccaggaacaaaacatactgaagaacggcccaaggtacaggtgatccaggctgccgatccgctagcaacagcg
tatctgtgcggaatccaccgagagctggttaggagattaaatgcggtcctgcttccgaacattcatacactgtttgatatgtcggctgaag
actttgacgctattatagccgagcacttccagcctggggattgtgttctggaaactgacatcgcgtcgtttgataaaagtgaggacgacg
ccatggctctgaccgcgttaatgattctggaagacttaggtgtggacgcagagctgttgacgctgattgaggcggctttcggcgaaattt
catcaatacatttgcccactaaaactaaatttaaattcggagccatgatgaaatctggaatgttcctcacactgtttgtgaacacagtcatta
acattgtaatcgcaagcagagtgttgagagaacggctaaccggatcaccatgtgcagcattcattggagatgacaatatcgtgaaagga
gtcaaatcggacaaattaatggcagacaggtgcgccacctggttgaatatggaagtcaagattatagatgctgtggtgggcgagaaag
cgccttatttctgtggagggtttattttgtgtgactccgtgaccggcacagcgtgccgtgtggcagaccccctaaaaaggctgtttaagctt
ggcaaacctctggcagcagacgatgaacatgatgatgacaggagaagggcattgcatgaagagtcaacacgctggaaccgagtgg
gtattctttcagagctgtgcaaggcagtagaatcaaggtatgaaaccgtaggaacttccatcatagttatggccatgactactctagctag
cagtgttaaatcattcagctacctgagaggggcccctataactctctacggcTAAcctgaatggactacgacgtatcacgcccaaac
atttacagccgcggtgtcaaaaaccgcgtggacgtggttaacatccctgctgggaggatcagccgtaattattataattggcttggtgctg
gctactattgtggccatgtacgtgctgaccaaccagaaacataattgaatacagcagcaattggcaagctgcttacatagaactcgcgg
cgattggcatgccgccttaaaatttttattttattttttcttttcttttccgaatcggattttgtttttaatatttcAAAAAAAAAAAAA
AAAAAAAAAAAAAAAAAAAAAAAAAAAAAAAAAAAAAAAAAAAAAAAAAAAA
AAAAAAAAAAAAAAA
TC-83 Delivery Vector (SEQ ID NO: 7); TC-83 genome with nucleotides 7544-11175 deleted [alphavirus
structural proteins removed
ATAGGCGGCGCATGAGAGAAGCCCAGACCAATTACCTACCCAAAATGGAGAAAGTTCACGTTGAC
ATCGAGGAAGACAGCCCATTCCTCAGAGCTTTGCAGCGGAGCTTCCCGCAGTTTGAGGTAGAAGCC
AAGCAGGTCACTGATAATGACCATGCTAATGCCAGAGCGTTTTCGCATCTGGCTTCAAAACTGATC
GAAACGGAGGTGGACCCATCCGACACGATCCTTGACATTGGAAGTGCGCCCGCCCGCAGAATGTA
TTCTAAGCACAAGTATCATTGTATCTGTCCGATGAGATGTGCGGAAGATCCGGACAGATTGTATAA
GTATGCAACTAAGCTGAAGAAAAACTGTAAGGAAATAACTGATAAGGAATTGGACAAGAAAATG
AAGGAGCTCGCCGCCGTCATGAGCGACCCTGACCTGGAAACTGAGACTATGTGCCTCCACGACGA
CGAGTCGTGTCGCTACGAAGGGCAAGTCGCTGTTTACCAGGATGTATACGCGGTTGACGGACCGAC
AAGTCTCTATCACCAAGCCAATAAGGGAGTTAGAGTCGCCTACTGGATAGGCTTTGACACCACCCC
TTTTATGTTTAAGAACTTGGCTGGAGCATATCCATCATACTCTACCAACTGGGCCGACGAAACCGT
GTTAACGGCTCGTAACATAGGCCTATGCAGCTCTGACGTTATGGAGCGGTCACGTAGAGGGATGTC
CATTCTTAGAAAGAAGTATTTGAAACCATCCAACAATGTTCTATTCTCTGTTGGCTCGACCATCTAC
CACGAGAAGAGGGACTTACTGAGGAGCTGGCACCTGCCGTCTGTATTTCACTTACGTGGCAAGCAA
AATTACACATGTCGGTGTGAGACTATAGTTAGTTGCGACGGGTACGTCGTTAAAAGAATAGCTATC
AGTCCAGGCCTGTATGGGAAGCCTTCAGGCTATGCTGCTACGATGCACCGCGAGGGATTCTTGTGC
TGCAAAGTGACAGACACATTGAACGGGGAGAGGGTCTCTTTTCCCGTGTGCACGTATGTGCCAGCT
ACATTGTGTGACCAAATGACTGGCATACTGGCAACAGATGTCAGTGCGGACGACGCGCAAAAACT
GCTGGTTGGGCTCAACCAGCGTATAGTCGTCAACGGTCGCACCCAGAGAAACACCAATACCATGA
AAAATTACCTTTTGCCCGTAGTGGCCCAGGCATTTGCTAGGTGGGCAAAGGAATATAAGGAAGATC
AAGAAGATGAAAGGCCACTAGGACTACGAGATAGACAGTTAGTCATGGGGTGTTGTTGGGCTTTT
AGAAGGCACAAGATAACATCTATTTATAAGCGCCCGGATACCCAAACCATCATCAAAGTGAACAG
CGATTTCCACTCATTCGTGCTGCCCAGGATAGGCAGTAACACATTGGAGATCGGGCTGAGAACAAG
AATCAGGAAAATGTTAGAGGAGCACAAGGAGCCGTCACCTCTCATTACCGCCGAGGACGTACAAG
AAGCTAAGTGCGCAGCCGATGAGGCTAAGGAGGTGCGTGAAGCCGAGGAGTTGCGCGCAGCTCTA
CCACCTTTGGCAGCTGATGTTGAGGAGCCCACTCTGGAAGCCGATGTCGACTTGATGTTACAAGAG
GCTGGGGCCGGCTCAGTGGAGACACCTCGTGGCTTGATAAAGGTTACCAGCTACGATGGCGAGGA
CAAGATCGGCTCTTACGCTGTGCTTTCTCCGCAGGCTGTACTCAAGAGTGAAAAATTATCTTGCATC
CACCCTCTCGCTGAACAAGTCATAGTGATAACACACTCTGGCCGAAAAGGGCGTTATGCCGTGGAA
CCATACCATGGTAAAGTAGTGGTGCCAGAGGGACATGCAATACCCGTCCAGGACTTTCAAGCTCTG
AGTGAAAGTGCCACCATTGTGTACAACGAACGTGAGTTCGTAAACAGGTACCTGCACCATATTGCC
ACACATGGAGGAGCGCTGAACACTGATGAAGAATATTACAAAACTGTCAAGCCCAGCGAGCACGA
CGGCGAATACCTGTACGACATCGACAGGAAACAGTGCGTCAAGAAAGAACTAGTCACTGGGCTAG
GGCTCACAGGCGAGCTGGTGGATCCTCCCTTCCATGAATTCGCCTACGAGAGTCTGAGAACACGAC
CAGCCGCTCCTTACCAAGTACCAACCATAGGGGTGTATGGCGTGCCAGGATCAGGCAAGTCTGGC
ATCATTAAAAGCGCAGTCACCAAAAAAGATCTAGTGGTGAGCGCCAAGAAAGAAAACTGTGCAGA
AATTATAAGGGACGTCAAGAAAATGAAAGGGCTGGACGTCAATGCCAGAACTGTGGACTCAGTGC
TCTTGAATGGATGCAAACACCCCGTAGAGACCCTGTATATTGACGAAGCTTTTGCTTGTCATGCAG
GTACTCTCAGAGCGCTCATAGCCATTATAAGACCTAAAAAGGCAGTGCTCTGCGGGGATCCCAAAC
AGTGCGGTTTTTTTAACATGATGTGCCTGAAAGTGCATTTTAACCACGAGATTTGCACACAAGTCTT
CCACAAAAGCATCTCTCGCCGTTGCACTAAATCTGTGACTTCGGTCGTCTCAACCTTGTTTTACGAC
AAAAAAATGAGAACGACGAATCCGAAAGAGACTAAGATTGTGATTGACACTACCGGCAGTACCAA
ACCTAAGCAGGACGATCTCATTCTCACTTGTTTCAGAGGGTGGGTGAAGCAGTTGCAAATAGATTA
CAAAGGCAACGAAATAATGACGGCAGCTGCCTCTCAAGGGCTGACCCGTAAAGGTGTGTATGCCG
TTCGGTACAAGGTGAATGAAAATCCTCTGTACGCACCCACCTCAGAACATGTGAACGTCCTACTGA
CCCGCACGGAGGACCGCATCGTGTGGAAAACACTAGCCGGCGACCCATGGATAAAAACACTGACT
GCCAAGTACCCTGGGAATTTCACTGCCACGATAGAGGAGTGGCAAGCAGAGCATGATGCCATCAT
GAGGCACATCTTGGAGAGACCGGACCCTACCGACGTCTTCCAGAATAAGGCAAACGTGTGTTGGG
CCAAGGCTTTAGTGCCGGTGCTGAAGACCGCTGGCATAGACATGACCACTGAACAATGGAACACT
GTGGATTATTTTGAAACGGACAAAGCTCACTCAGCAGAGATAGTATTGAACCAACTATGCGTGAG
GTTCTTTGGACTCGATCTGGACTCCGGTCTATTTTCTGCACCCACTGTTCCGTTATCCATTAGGAAT
AATCACTGGGATAACTCCCCGTCGCCTAACATGTACGGGCTGAATAAAGAAGTGGTCCGTCAGCTC
TCTCGCAGGTACCCACAACTGCCTCGGGCAGTTGCCACTGGAAGAGTCTATGACATGAACACTGGT
ACACTGCGCAATTATGATCCGCGCATAAACCTAGTACCTGTAAACAGAAGACTGCCTCATGCTTTA
GTCCTCCACCATAATGAACACCCACAGAGTGACTTTTCTTCATTCGTCAGCAAATTGAAGGGCAGA
ACTGTCCTGGTGGTCGGGGAAAAGTTGTCCGTCCCAGGCAAAATGGTTGACTGGTTGTCAGACCGG
CCTGAGGCTACCTTCAGAGCTCGGCTGGATTTAGGCATCCCAGGTGATGTGCCCAAATATGACATA
ATATTTGTTAATGTGAGGACCCCATATAAATACCATCACTATCAGCAGTGTGAAGACCATGCCATT
AAGCTTAGCATGTTGACCAAGAAAGCTTGTCTGCATCTGAATCCCGGCGGAACCTGTGTCAGCATA
GGTTATGGTTACGCTGACAGGGCCAGCGAAAGCATCATTGGTGCTATAGCGCGGCAGTTCAAGTTT
TCCCGGGTATGCAAACCGAAATCCTCACTTGAAGAGACGGAAGTTCTGTTTGTATTCATTGGGTAC
GATCGCAAGGCCCGTACGCACAATCCTTACAAGCTTTCATCAACCTTGACCAACATTTATACAGGT
TCCAGACTCCACGAAGCCGGATGTGCACCCTCATATCATGTGGTGCGAGGGGATATTGCCACGGCC
ACCGAAGGAGTGATTATAAATGCTGCTAACAGCAAAGGACAACCTGGCGGAGGGGTGTGCGGAGC
GCTGTATAAGAAATTCCCGGAAAGCTTCGATTTACAGCCGATCGAAGTAGGAAAAGCGCGACTGG
TCAAAGGTGCAGCTAAACATATCATTCATGCCGTAGGACCAAACTTCAACAAAGTTTCGGAGGTTG
AAGGTGACAAACAGTTGGCAGAGGCTTATGAGTCCATCGCTAAGATTGTCAACGATAACAATTAC
AAGTCAGTAGCGATTCCACTGTTGTCCACCGGCATCTTTTCCGGGAACAAAGATCGACTAACCCAA
TCATTGAACCATTTGCTGACAGCTTTAGACACCACTGATGCAGATGTAGCCATATACTGCAGGGAC
AAGAAATGGGAAATGACTCTCAAGGAAGCAGTGGCTAGGAGAGAAGCAGTGGAGGAGATATGCA
TATCCGACGACTCTTCAGTGACAGAACCTGATGCAGAGCTGGTGAGGGTGCATCCGAAGAGTTCTT
TGGCTGGAAGGAAGGGCTACAGCACAAGCGATGGCAAAACTTTCTCATATTTGGAAGGGACCAAG
TTTCACCAGGCGGCCAAGGATATAGCAGAAATTAATGCCATGTGGCCCGTTGCAACGGAGGCCAA
TGAGCAGGTATGCATGTATATCCTCGGAGAAAGCATGAGCAGTATTAGGTCGAAATGCCCCGTCG
AAGAGTCGGAAGCCTCCACACCACCTAGCACGCTGCCTTGCTTGTGCATCCATGCCATGACTCCAG
AAAGAGTACAGCGCCTAAAAGCCTCACGTCCAGAACAAATTACTGTGTGCTCATCCTTTCCATTGC
CGAAGTATAGAATCACTGGTGTGCAGAAGATCCAATGCTCCCAGCCTATATTGTTCTCACCGAAAG
TGCCTGCGTATATTCATCCAAGGAAGTATCTCGTGGAAACACCACCGGTAGACGAGACTCCGGAGC
CATCGGCAGAGAACCAATCCACAGAGGGGACACCTGAACAACCACCACTTATAACCGAGGATGAG
ACCAGGACTAGAACGCCTGAGCCGATCATCATCGAAGAGGAAGAAGAGGATAGCATAAGTTTGCT
GTCAGATGGCCCGACCCACCAGGTGCTGCAAGTCGAGGCAGACATTCACGGGCCGCCCTCTGTATC
TAGCTCATCCTGGTCCATTCCTCATGCATCCGACTTTGATGTGGACAGTTTATCCATACTTGACACC
CTGGAGGGAGCTAGCGTGACCAGCGGGGCAACGTCAGCCGAGACTAACTCTTACTTCGCAAAGAG
TATGGAGTTTCTGGCGCGACCGGTGCCTGCGCCTCGAACAGTATTCAGGAACCCTCCACATCCCGC
TCCGCGCACAAGAACACCGTCACTTGCACCCAGCAGGGCCTGCTCGAGAACCAGCCTAGTTTCCAC
CCCGCCAGGCGTGAATAGGGTGATCACTAGAGAGGAGCTCGAGGCGCTTACCCCGTCACGCACTC
CTAGCAGGTCGGTCTCGAGAACCAGCCTGGTCTCCAACCCGCCAGGCGTAAATAGGGTGATTACA
AGAGAGGAGTTTGAGGCGTTCGTAGCACAACAACAATGACGGTTTGATGCGGGTGCATACATCTTT
TCCTCCGACACCGGTCAAGGGCATTTACAACAAAAATCAGTAAGGCAAACGGTGCTATCCGAAGT
GGTGTTGGAGAGGACCGAATTGGAGATTTCGTATGCCCCGCGCCTCGACCAAGAAAAAGAAGAAT
TACTACGCAAGAAATTACAGTTAAATCCCACACCTGCTAACAGAAGCAGATACCAGTCCAGGAAG
GTGGAGAACATGAAAGCCATAACAGCTAGACGTATTCTGCAAGGCCTAGGGCATTATTTGAAGGC
AGAAGGAAAAGTGGAGTGCTACCGAACCCTGCATCCTGTTCCTTTGTATTCATCTAGTGTGAACCG
TGCCTTTTCAAGCCCCAAGGTCGCAGTGGAAGCCTGTAACGCCATGTTGAAAGAGAACTTTCCGAC
TGTGGCTTCTTACTGTATTATTCCAGAGTACGATGCCTATTTGGACATGGTTGACGGAGCTTCATGC
TGCTTAGACACTGCCAGTTTTTGCCCTGCAAAGCTGCGCAGCTTTCCAAAGAAACACTCCTATTTGG
AACCCACAATACGATCGGCAGTGCCTTCAGCGATCCAGAACACGCTCCAGAACGTCCTGGCAGCT
GCCACAAAAAGAAATTGCAATGTCACGCAAATGAGAGAATTGCCCGTATTGGATTCGGCGGCCTTT
AATGTGGAATGCTTCAAGAAATATGCGTGTAATAATGAATATTGGGAAACGTTTAAAGAAAACCC
CATCAGGCTTACTGAAGAAAACGTGGTAAATTACATTACCAAATTAAAAGGACCAAAAGCTGCTG
CTCTTTTTGCGAAGACACATAATTTGAATATGTTGCAGGACATACCAATGGACAGGTTTGTAATGG
ACTTAAAGAGAGACGTGAAAGTGACTCCAGGAACAAAACATACTGAAGAACGGCCCAAGGTACA
GGTGATCCAGGCTGCCGATCCGCTAGCAACAGCGTATCTGTGCGGAATCCACCGAGAGCTGGTTAG
GAGATTAAATGCGGTCCTGCTTCCGAACATTCATACACTGTTTGATATGTCGGCTGAAGACTTTGA
CGCTATTATAGCCGAGCACTTCCAGCCTGGGGATTGTGTTCTGGAAACTGACATCGCGTCGTTTGA
TAAAAGTGAGGACGACGCCATGGCTCTGACCGCGTTAATGATTCTGGAAGACTTAGGTGTGGACG
CAGAGCTGTTGACGCTGATTGAGGCGGCTTTCGGCGAAATTTCATCAATACATTTGCCCACTAAAA
CTAAATTTAAATTCGGAGCCATGATGAAATCTGGAATGTTCCTCACACTGTTTGTGAACACAGTCA
TTAACATTGTAATCGCAAGCAGAGTGTTGAGAGAACGGCTAACCGGATCACCATGTGCAGCATTCA
TTGGAGATGACAATATCGTGAAAGGAGTCAAATCGGACAAATTAATGGCAGACAGGTGCGCCACC
TGGTTGAATATGGAAGTCAAGATTATAGATGCTGTGGTGGGCGAGAAAGCGCCTTATTTCTGTGGA
GGGTTTATTTTGTGTGACTCCGTGACCGGCACAGCGTGCCGTGTGGCAGACCCCCTAAAAAGGCTG
TTTAAGCTTGGCAAACCTCTGGCAGCAGACGATGAACATGATGATGACAGGAGAAGGGCATTGCA
TGAAGAGTCAACACGCTGGAACCGAGTGGGTATTCTTTCAGAGCTGTGCAAGGCAGTAGAATCAA
GGTATGAAACCGTAGGAACTTCCATCATAGTTATGGCCATGACTACTCTAGCTAGCAGTGTTAAAT
CATTCAGCTACCTGAGAGGGGCCCCTATAACTCTCTACGGCTAACCTGAATGGACTACGACATAGT
CTAGTCCGCCAAGATGTTCCCGTTCCAGCCAATGTATCCGATGCAGCCAATGCCCTATCGCAACCC
GTTCGCGGCCCCGCGCAGGCCCTGGTTCCCCAGAACCGACCCTTTTCTGGCGATGCAGGTGCAGGA
ATTAACCCGCTCGATGGCTAACCTGACGTTCAAGCAACGCCGGGACGCGCCACCTGAGGGGCCAT
CCGCTAAGAAACCGAAGAAGGAGGCCTCGCAAAAACAGAAAGGGGGAGGCCAAGGGAAGAAGA
AGAAGAACCAAGGGAAGAAGAAGGCTAAGACAGGGCCGCCTAATCCGAAGGCACAGAATGGAAA
CAAGAAGAAGACCAACAAGAAACCAGGCAAGAGACAGCGCATGGTCATGAAATTGGAATCTGAC
AAGACGTTCCCAATCATGTTGGAAGGGAAGATAAACGGCTACGCTTGTGTGGTCGGAGGGAAGTT
ATTCAGGCCGATGCATGTGGAAGGCAAGATCGACAACGACGTTCTGGCCGCGCTTAAGACGAAGA
AAGCATCCAAATACGATCTTGAGTATGCAGATGTGCCACAGAACATGCGGGCCGATACATTCAAA
TACACCCATGAGAAACCCCAAGGCTATTACAGCTGGCATCATGGAGCAGTCCAATATGAAAATGG
GCGTTTCACGGTGCCGAAAGGAGTTGGGGCCAAGGGAGACAGCGGACGACCCATTCTGGATAACC
AGGGACGGGTGGTCGCTATTGTGCTGGGAGGTGTGAATGAAGGATCTAGGACAGCCCTTTCAGTC
GTCATGTGGAACGAGAAGGGAGTTACCGTGAAGTATACTCCGGAGAACTGCGAGCAATGGTCACT
AGTGACCACCATGTGTCTGCTCGCCAATGTGACGTTCCCATGTGCTCAACCACCAATTTGCTACGA
CAGAAAACCAGCAGAGACTTTGGCCATGCTCAGCGTTAACGTTGACAACCCGGGCTACGATGAGC
TGCTGGAAGCAGCTGTTAAGTGCCCCGGAAGGAAAAGGAGATCCACCGAGGAGCTGTTTAATGAG
TATAAGCTAACGCGCCCTTACATGGCCAGATGCATCAGATGTGCAGTTGGGAGCTGCCATAGTCCA
ATAGCAATCGAGGCAGTAAAGAGCGACGGGCACGACGGTTATGTTAGACTTCAGACTTCCTCGCA
GTATGGCCTGGATTCCTCCGGCAACTTAAAGGGCAGGACCATGCGGTATGACATGCACGGGACCA
TTAAAGAGATACCACTACATCAAGTGTCACTCTATACATCTCGCCCGTGTCACATTGTGGATGGGC
ACGGTTATTTCCTGCTTGCCAGGTGCCCGGCAGGGGACTCCATCACCATGGAATTTAAGAAAGATT
CCGTCAGACACTCCTGCTCGGTGCCGTATGAAGTGAAATTTAATCCTGTAGGCAGAGAACTCTATA
CTCATCCCCCAGAACACGGAGTAGAGCAAGCGTGCCAAGTCTACGCACATGATGCACAGAACAGA
GGAGCTTATGTCGAGATGCACCTCCCGGGCTCAGAAGTGGACAGCAGTTTGGTTTCCTTGAGCGGC
AGTTCAGTCACCGTGACACCTCCTGATGGGACTAGCGCCCTGGTGGAATGCGAGTGTGGCGGCACA
AAGATCTCCGAGACCATCAACAAGACAAAACAGTTCAGCCAGTGCACAAAGAAGGAGCAGTGCA
GAGCATATCGGCTGCAGAACGATAAGTGGGTGTATAATTCTGACAAACTGCCCAAAGCAGCGGGA
GCCACCTTAAAAGGAAAACTGCATGTCCCATTCTTGCTGGCAGACGGCAAATGCACCGTGCCTCTA
GCACCAGAACCTATGATAACCTTCGGTTTCAGATCAGTGTCACTGAAACTGCACCCTAAGAATCCC
ACATATCTAATCACCCGCCAACTTGCTGATGAGCCTCACTACACGCACGAGCTCATATCTGAACCA
GCTGTTAGGAATTTTACCGTCACCGAAAAAGGGTGGGAGTTTGTATGGGGAAACCACCCGCCGAA
AAGGTTTTGGGCACAGGAAACAGCACCCGGAAATCCACATGGGCTACCGCACGAGGTGATAACTC
ATTATTACCACAGATACCCTATGTCCACCATCCTGGGTTTGTCAATTTGTGCCGCCATTGCAACCGT
TTCCGTTGCAGCGTCTACCTGGCTGTTTTGCAGATCTAGAGTTGCGTGCCTAACTCCTTACCGGCTA
ACACCTAACGCTAGGATACCATTTTGTCTGGCTGTGCTTTGCTGCGCCCGCACTGCCCGGGCCGAG
ACCACCTGGGAGTCCTTGGATCACCTATGGAACAATAACCAACAGATGTTCTGGATTCAATTGCTG
ATCCCTCTGGCCGCCTTGATCGTAGTGACTCGCCTGCTCAGGTGCGTGTGCTGTGTCGTGCCTTTTT
TAGTCATGGCCGGCGCCGCAGGCGCCGGCGCCTACGAGCACGCGACCACGATGCCGAGCCAAGCG
GGAATCTCGTATAACACTATAGTCAACAGAGCAGGCTACGCACCACTCCCTATCAGCATAACACCA
ACAAAGATCAAGCTGATACCTACAGTGAACTTGGAGTACGTCACCTGCCACTACAAAACAGGAAT
GGATTCACCAGCCATCAAATGCTGCGGATCTCAGGAATGCACTCCAACTTACAGGCCTGATGAACA
GTGCAAAGTCTTCACAGGGGTTTACCCGTTCATGTGGGGTGGTGCATATTGCTTTTGCGACACTGA
GAACACCCAAGTCAGCAAGGCCTACGTAATGAAATCTGACGACTGCCTTGCGGATCATGCTGAAG
CATATAAAGCGCACACAGCCTCAGTGCAGGCGTTCCTCAACATCACAGTGGGAGAACACTCTATTG
TGACTACCGTGTATGTGAATGGAGAAACTCCTGTGAATTTCAATGGGGTCAAAATAACTGCAGGTC
CGCTTTCCACAGCTTGGACACCCTTTGATCGCAAAATCGTGCAGTATGCCGGGGAGATCTATAATT
ATGATTTTCCTGAGTATGGGGCAGGACAACCAGGAGCATTTGGAGATATACAATCCAGAACAGTCT
CAAGCTCTGATCTGTATGCCAATACCAACCTAGTGCTGCAGAGACCCAAAGCAGGAGCGATCCAC
GTGCCATACACTCAGGCACCTTCGGGTTTTGAGCAATGGAAGAAAGATAAAGCTCCATCATTGAAA
TTTACCGCCCCTTTCGGATGCGAAATATATACAAACCCCATTCGCGCCGAAAACTGTGCTGTAGGG
TCAATTCCATTAGCCTTTGACATTCCCGACGCCTTGTTCACCAGGGTGTCAGAAACACCGACACTTT
CAGCGGCCGAATGCACTCTTAACGAGTGCGTGTATTCTTCCGACTTTGGTGGGATCGCCACGGTCA
AGTACTCGGCCAGCAAGTCAGGCAAGTGCGCAGTCCATGTGCCATCAGGGACTGCTACCCTAAAA
GAAGCAGCAGTCGAGCTAACCGAGCAAGGGTCGGCGACTATCCATTTCTCGACCGCAAATATCCA
CCCGGAGTTCAGGCTCCAAATATGCACATCATATGTTACGTGCAAAGGTGATTGTCACCCCCCGAA
AGACCATATTGTGACACACCCTCAGTATCACGCCCAAACATTTACAGCCGCGGTGTCAAAAACCGC
GTGGACGTGGTTAACATCCCTGCTGGGAGGATCAGCCGTAATTATTATAATTGGCTTGGTGCTGGC
TACTATTGTGGCCATGTACGTGCTGACCAACCAGAAACATAATTGAATACAGCAGCAATTGGCAAG
CTGCTTACATAGAACTCGCGGCGATTGGCATGCCGCCTTAAAATTTTTATTTTATTTTTCTTTTCTTT
TCCGAATCGGATTTTGTTTTTAATATTTC
VEE Production Vector (SEQ ID NO: 8); VEE genome with nucleotides 7544-11175 deleted, plus 5′ T7-
promoter, plus 3′ restriction sites
TAATACGACTCACTATAGGATGggcggcgcatgagagaagcccagaccaattacctacccaaaATGGagaaagttcacgttgacatcga
ggaagacagcccattcctcagagctttgcagcggagcttcccgcagtttgaggtagaagccaagcaggtcactgataatgaccatgctaatgccagagcgttttcg
catctggcttcaaaactgatcgaaacggaggtggacccatccgacacgatccttgacattggaagtgcgcccgcccgcagaatgtattctaagcacaagtatcattg
tatctgtccgatgagatgtgcggaagatccggacagattgtataagtatgcaactaagctgaagaaaaactgtaaggaaataactgataaggaattggacaagaaaa
tgaaggagctcgccgccgtcatgagcgaccctgacctggaaactgagactatgtgcctccacgacgacgagtcgtgtcgctacgaagggcaagtcgctgtttacc
aggatgtatacgcggttgacggaccgacaagtctctatcaccaagccaataagggagttagagtcgcctactggataggctttgacaccaccccttttatgtttaaga
acttggctggagcatatccatcatactctaccaactgggccgacgaaaccgtgttaacggctcgtaacataggcctatgcagctctgacgttatggagcggtcacgt
agagggatgtccattcttagaaagaagtatttgaaaccatccaacaatgttctattctctgttggctcgaccatctaccacgagaagagggacttactgaggagctggc
acctgccgtctgtatttcacttacgtggcaagcaaaattacacatgtcggtgtgagactatagttagttgcgacgggtacgtcgttaaaagaatagctatcagtccagg
cctgtatgggaagccttcaggctatgctgctacgatgcaccgcgagggattcttgtgctgcaaagtgacagacacattgaacggggagagggtctcttttcccgtgt
gcacgtatgtgccagctacattgtgtgaccaaatgactggcatactggcaacagatgtcagtgcggacgacgcgcaaaaactgctggttgggctcaaccagcgtat
agtcgtcaacggtcgcacccagagaaacaccaataccatgaaaaattaccttttgcccgtagtggcccaggcatttgctaggtgggcaaaggaatataaggaagat
caagaagatgaaaggccactaggactacgagatagacagttagtcatggggtgttgttgggcttttagaaggcacaagataacatctatttataagcgcccggatac
ccaaaccatcatcaaagtgaacagcgatttccactcattcgtgctgcccaggataggcagtaacacattggagatcgggctgagaacaagaatcaggaaaatgtta
gaggagcacaaggagccgtcacctctcattaccgccgaggacgtacaagaagctaagtgcgcagccgatgaggctaaggaggtgcgtgaagccgaggagttg
cgcgcagctctaccacctttggcagctgatgttgaggagcccactctggaagccgatgtcgacttgatgttacaagaggctggggccggctcagtggagacacctc
gtggcttgataaaggttaccagctacgctggcgaggacaagatcggctcttacgctgtgctttctccgcaggctgtactcaagagtgaaaaattatcttgcatccaccc
tctcgctgaacaagtcatagtgataacacactctggccgaaaagggcgttatgccgtggaaccataccatggtaaagtagtggtgccagagggacatgcaataccc
gtccaggactttcaagctctgagtgaaagtgccaccattgtgtacaacgaacgtgagttcgtaaacaggtacctgcaccatattgccacacatggaggagcgctgaa
cactgatgaagaatattacaaaactgtcaagcccagcgagcacgacggcgaatacctgtacgacatcgacaggaaacagtgcgtcaagaaagaactagtcactg
ggctagggctcacaggcgagctggtggatcctcccttccatgaattcgcctacgagagtctgagaacacgaccagccgctccttaccaagtaccaaccatagggg
tgtatggcgtgccaggatcaggcaagtctggcatcattaaaagcgcagtcaccaaaaaagatctagtggtgagcgccaagaaagaaaactgtgcagaaattataa
gggacgtcaagaaaatgaaagggctggacgtcaatgccagaactgtggactcagtgctcttgaatggatgcaaacaccccgtagagaccctgtatattgacgaag
cttttgcttgtcatgcaggtactctcagagcgctcatagccattataagacctaaaaaggcagtgctctgcggggatcccaaacagtgcggtttttttaacatgatgtgc
ctgaaagtgcattttaaccacgagatttgcacacaagtcttccacaaaagcatctctcgccgttgcactaaatctgtgacttcggtcgtctcaaccttgttttacgacaaa
aaaatgagaacgacgaatccgaaagagactaagattgtgattgacactaccggcagtaccaaacctaagcaggacgatctcattctcacttgtttcagaggggggt
gaagcagttgcaaatagattacaaaggcaacgaaataatgacggcagctgcctctcaagggctgacccgtaaaggtgtgtatgccgttcggtacaaggtgaatgaa
aatcctctgtacgcacccacctcagaacatgtgaacgtcctactgacccgcacggaggaccgcatcgtgtggaaaacactagccggcgacccatggataaaaaca
ctgactgccaagtaccctgggaatttcactgccacgatagaggagtggcaagcagagcatgatgccatcatgaggcacatcttggagagaccggaccctaccgac
gtcttccagaataaggcaaacgtgtgttgggccaaggctttagtgccggtgctgaagaccgctggcatagacatgaccactgaacaatggaacactgtggattatttt
gaaacggacaaagctcactcagcagagatagtattgaaccaactatgcgtgaggttctttggactcgatctggactccggtctattttctgcacccactgttccgttatc
cattaggaataatcactgggataactccccgtcgcctaacatgtacgggctgaataaagaagtggtccgtcagctctctcgcaggtacccacaactgcctcgggca
gttgccactggaagagtctatgacatgaacactggtacactgcgcaattatgatccgcgcataaacctagtacctgtaaacagaagactgcctcatgctttagtcctcc
accataatgaacacccacagagtgacttttcttcattcgtcagcaaattgaagggcagaactgtcctggtggtcggggaaaagttgtccgtcccaggcaaaatggttg
actggttgtcagaccggcctgaggctaccttcagagctcggctggatttaggcatcccaggtgatgtgcccaaatatgacataatatttgttaatgtgaggaccccata
taaataccatcactatcagcagtgtgaagaccatgccattaagcttagcatgttgaccaagaaagcttgtctgcatctgaatcccggcggaacctgtgtcagcataggt
tatggttacgctgacagggccagcgaaagcatcattggtgctatagcgcggcagttcaagttttcccgggtatgcaaaccgaaatcctcacttgaagagacggaagt
tctgtttgtattcattgggtacgatcgcaaggcccgtacgcacaatccttacaagctttcatcaaccttgaccaacatttatacaggttccagactccacgaagccggat
gtgcaccctcatatcatgtggtgcgaggggatattgccacggccaccgaaggagtgattataaatgctgctaacagcaaaggacaacctggcggaggggtgtgcg
gagcgctgtataagaaattcccggaaagcttcgatttacagccgatcgaagtaggaaaagcgcgactggtcaaaggtgcagctaaacatatcattcatgccgtagg
accaaacttcaacaaagtttcggaggttgaaggtgacaaacagttggcagaggcttatgagtccatcgctaagattgtcaacgataacaattacaagtcagtagcgat
tccactgttgtccaccggcatcttttccgggaacaaagatcgactaacccaatcattgaaccatttgctgacagctttagacaccactgatgcagatgtagccatatact
gcagggacaagaaatgggaaatgactctcaaggaagcagtggctaggagagaagcagtggaggagatatgcatatccgacgactcttcagtgacagaacctgat
gcagagctggtgagggtgcatccgaagagttctttggctggaaggaagggctacagcacaagcgatggcaaaactttctcatatttggaagggaccaagtttcacc
aggcggccaaggatatagcagaaattaatgccatgtggcccgttgcaacggaggccaatgagcaggtatgcatgtatatcctcggagaaagcatgagcagtatta
ggtcgaaatgccccgtcgaagagtcggaagcctccacaccacctagcacgctgccttgcttgtgcatccatgccatgactccagaaagagtacagcgcctaaaag
cctcacgtccagaacaaattactgtgtgctcatcctttccattgccgaagtatagaatcactggtgtgcagaagatccaatgctcccagcctatattgttctcaccgaaa
gtgcctgcgtatattcatccaaggaagtatctcgtggaaacaccaccggtagacgagactccggagccatcggcagagaaccaatccacagaggggacacctga
acaaccaccacttataaccgaggatgagaccaggactagaacgcctgagccgatcatcatcgaagaggaagaagaggatagcataagtttgctgtcagatggcc
cgacccaccaggtgctgcaagtcgaggcagacattcacgggccgccctctgtatctagctcatcctggtccattcctcatgcatccgactttgatgtggacagtttatc
catacttgacaccctggagggagctagcgtgaccagcggggcaacgtcagccgagactaactcttacttcgcaaagagtatggagtttctggcgcgaccggtgcc
tgcgcctcgaacagtattcaggaaccctccacatcccgctccgcgcacaagaacaccgtcacttgcacccagcagggcctgctcgagaaccagcctagtttccac
cccgccaggcgtgaatagggtgatcactagagaggagctcgaggcgcttaccccgtcacgcactcctagcaggtcggtctcgagaaccagcctggtctccaacc
cgccaggcgtaaatagggtgattacaagagaggagtttgaggcgttcgtagcacaacaacaatgacggtttgatgcgggtgcatacatcttttcctccgacaccggt
caagggcatttacaacaaaaatcagtaaggcaaacggtgctatccgaagtggtgttggagaggaccgaattggagatttcgtatgccccgcgcctcgaccaagaa
aaagaagaattactacgcaagaaattacagttaaatcccacacctgctaacagaagcagataccagtccaggaaggtggagaacatgaaagccataacagctaga
cgtattctgcaaggcctagggcattatttgaaggcagaaggaaaagtggagtgctaccgaaccctgcatcctgttcctttgtattcatctagtgtgaaccgtgccttttc
aagccccaaggtcgcagtggaagcctgtaacgccatgttgaaagagaactttccgactgtggcttcttactgtattattccagagtacgatgcctatttggacatggttg
acggagcttcatgctgcttagacactgccagtttttgccctgcaaagctgcgcagctttccaaagaaacactcctatttggaacccacaatacgatcggcagtgccttc
agcgatccagaacacgctccagaacgtcctggcagctgccacaaaaagaaattgcaatgtcacgcaaatgagagaattgcccgtattggattcggggcctttaat
gtggaatgcttcaagaaatatgcgtgtaataatgaatattgggaaacgtttaaagaaaaccccatcaggcttactgaagaaaacgtggtaaattacattaccaaattaa
aaggaccaaaagctgctgctctttttgcgaagacacataatttgaatatgttgcaggacataccaatggacaggtttgtaatggacttaaagagagacgtgaaagtga
ctccaggaacaaaacatactgaagaacggcccaaggtacaggtgatccaggctgccgatccgctagcaacagcgtatctgtgcggaatccaccgagagctggtt
aggagattaaatgcggtcctgcttccgaacattcatacactgtttgatatgtcggctgaagactttgacgctattatagccgagcacttccagcctggggattgtgttctg
gaaactgacatcgcgtcgtttgataaaagtgaggacgacgccatggctctgaccgcgttaatgattctggaagacttaggtgtggacgcagagctgttgacgctgat
tgaggcggctttcggcgaaatttcatcaatacatttgcccactaaaactaaatttaaattcggagccatgatgaaatctggaatgttcctcacactgtttgtgaacacagt
cattaacattgtaatcgcaagcagagtgttgagagaacggctaaccggatcaccatgtgcagcattcattggagatgacaatatcgtgaaaggagtcaaatcggaca
aattaatggcagacaggtgcgccacctggttgaatatggaagtcaagattatagatgctgtggtgggcgagaaagcgccttatttctgtggagggtttattttgtgtga
ctccgtgaccggcacagcgtgccgtgtggcagaccccctaaaaaggctgtttaagcttggcaaacctctggcagcagacgatgaacatgatgatgacaggagaa
gggcattgcatgaagagtcaacacgctggaaccgagtgggtattctttcagagctgtgcaaggcagtagaatcaaggtatgaaaccgtaggaacttccatcatagtt
atggccatgactactctagctagcagtgttaaatcattcagctacctgagaggggcccctataactctctacggcTAAcctgaatggactacgacgtatcacgccc
aaacatttacagccgcggtgtcaaaaaccgcgtggacgtggttaacatccctgctgggaggatcagccgtaattattataattggcttggtgctggctactattgtggc
catgtacgtgctgaccaaccagaaacataattgaatacagcagcaattggcaagctgcttacatagaactcgcggcgattggcatgccgccttaaaatttttattttattt
tttcttttcttttccgaatcggattttgtttttaatatttcAAAAAAAAAAAAAAAAAAAAAAAAAAAAAAAAAAAAAAAAAA
AAAAAAAAAAAAAAAAAAAAAAAAAAAAAAAAAAAAAA
TC-83 Production Vector (SEQ ID NO: 9); TC-83 genome with nucleotides 7544-11175 deleted, plus 5′ T7-
promoter, plus 3′ restriction sites
ATAGGCGGCGCATGAGAGAAGCCCAGACCAATTACCTACCCAAAATGGAGAAAGTTCACGTTGAC
ATCGAGGAAGACAGCCCATTCCTCAGAGCTTTGCAGCGGAGCTTCCCGCAGTTTGAGGTAGAAGCC
AAGCAGGTCACTGATAATGACCATGCTAATGCCAGAGCGTTTTCGCATCTGGCTTCAAAACTGATC
GAAACGGAGGTGGACCCATCCGACACGATCCTTGACATTGGAAGTGCGCCCGCCCGCAGAATGTA
TTCTAAGCACAAGTATCATTGTATCTGTCCGATGAGATGTGCGGAAGATCCGGACAGATTGTATAA
GTATGCAACTAAGCTGAAGAAAAACTGTAAGGAAATAACTGATAAGGAATTGGACAAGAAAATG
AAGGAGCTCGCCGCCGTCATGAGCGACCCTGACCTGGAAACTGAGACTATGTGCCTCCACGACGA
CGAGTCGTGTCGCTACGAAGGGCAAGTCGCTGTTTACCAGGATGTATACGCGGTTGACGGACCGAC
AAGTCTCTATCACCAAGCCAATAAGGGAGTTAGAGTCGCCTACTGGATAGGCTTTGACACCACCCC
TTTTATGTTTAAGAACTTGGCTGGAGCATATCCATCATACTCTACCAACTGGGCCGACGAAACCGT
GTTAACGGCTCGTAACATAGGCCTATGCAGCTCTGACGTTATGGAGCGGTCACGTAGAGGGATGTC
CATTCTTAGAAAGAAGTATTTGAAACCATCCAACAATGTTCTATTCTCTGTTGGCTCGACCATCTAC
CACGAGAAGAGGGACTTACTGAGGAGCTGGCACCTGCCGTCTGTATTTCACTTACGTGGCAAGCAA
AATTACACATGTCGGTGTGAGACTATAGTTAGTTGCGACGGGTACGTCGTTAAAAGAATAGCTATC
AGTCCAGGCCTGTATGGGAAGCCTTCAGGCTATGCTGCTACGATGCACCGCGAGGGATTCTTGTGC
TGCAAAGTGACAGACACATTGAACGGGGAGAGGGTCTCTTTTCCCGTGTGCACGTATGTGCCAGCT
ACATTGTGTGACCAAATGACTGGCATACTGGCAACAGATGTCAGTGCGGACGACGCGCAAAAACT
GCTGGTTGGGCTCAACCAGCGTATAGTCGTCAACGGTCGCACCCAGAGAAACACCAATACCATGA
AAAATTACCTTTTGCCCGTAGTGGCCCAGGCATTTGCTAGGTGGGCAAAGGAATATAAGGAAGATC
AAGAAGATGAAAGGCCACTAGGACTACGAGATAGACAGTTAGTCATGGGGTGTTGTTGGGCTTTT
AGAAGGCACAAGATAACATCTATTTATAAGCGCCCGGATACCCAAACCATCATCAAAGTGAACAG
CGATTTCCACTCATTCGTGCTGCCCAGGATAGGCAGTAACACATTGGAGATCGGGCTGAGAACAAG
AATCAGGAAAATGTTAGAGGAGCACAAGGAGCCGTCACCTCTCATTACCGCCGAGGACGTACAAG
AAGCTAAGTGCGCAGCCGATGAGGCTAAGGAGGTGCGTGAAGCCGAGGAGTTGCGCGCAGCTCTA
CCACCTTTGGCAGCTGATGTTGAGGAGCCCACTCTGGAAGCCGATGTCGACTTGATGTTACAAGAG
GCTGGGGCCGGCTCAGTGGAGACACCTCGTGGCTTGATAAAGGTTACCAGCTACGATGGCGAGGA
CAAGATCGGCTCTTACGCTGTGCTTTCTCCGCAGGCTGTACTCAAGAGTGAAAAATTATCTTGCATC
CACCCTCTCGCTGAACAAGTCATAGTGATAACACACTCTGGCCGAAAAGGGCGTTATGCCGTGGAA
CCATACCATGGTAAAGTAGTGGTGCCAGAGGGACATGCAATACCCGTCCAGGACTTTCAAGCTCTG
AGTGAAAGTGCCACCATTGTGTACAACGAACGTGAGTTCGTAAACAGGTACCTGCACCATATTGCC
ACACATGGAGGAGCGCTGAACACTGATGAAGAATATTACAAAACTGTCAAGCCCAGCGAGCACGA
CGGCGAATACCTGTACGACATCGACAGGAAACAGTGCGTCAAGAAAGAACTAGTCACTGGGCTAG
GGCTCACAGGCGAGCTGGTGGATCCTCCCTTCCATGAATTCGCCTACGAGAGTCTGAGAACACGAC
CAGCCGCTCCTTACCAAGTACCAACCATAGGGGTGTATGGCGTGCCAGGATCAGGCAAGTCTGGC
ATCATTAAAAGCGCAGTCACCAAAAAAGATCTAGTGGTGAGCGCCAAGAAAGAAAACTGTGCAGA
AATTATAAGGGACGTCAAGAAAATGAAAGGGCTGGACGTCAATGCCAGAACTGTGGACTCAGTGC
TCTTGAATGGATGCAAACACCCCGTAGAGACCCTGTATATTGACGAAGCTTTTGCTTGTCATGCAG
GTACTCTCAGAGCGCTCATAGCCATTATAAGACCTAAAAAGGCAGTGCTCTGCGGGGATCCCAAAC
AGTGCGGTTTTTTTAACATGATGTGCCTGAAAGTGCATTTTAACCACGAGATTTGCACACAAGTCTT
CCACAAAAGCATCTCTCGCCGTTGCACTAAATCTGTGACTTCGGTCGTCTCAACCTTGTTTTACGAC
AAAAAAATGAGAACGACGAATCCGAAAGAGACTAAGATTGTGATTGACACTACCGGCAGTACCAA
ACCTAAGCAGGACGATCTCATTCTCACTTGTTTCAGAGGGTGGGTGAAGCAGTTGCAAATAGATTA
CAAAGGCAACGAAATAATGACGGCAGCTGCCTCTCAAGGGCTGACCCGTAAAGGTGTGTATGCCG
TTCGGTACAAGGTGAATGAAAATCCTCTGTACGCACCCACCTCAGAACATGTGAACGTCCTACTGA
CCCGCACGGAGGACCGCATCGTGTGGAAAACACTAGCCGGCGACCCATGGATAAAAACACTGACT
GCCAAGTACCCTGGGAATTTCACTGCCACGATAGAGGAGTGGCAAGCAGAGCATGATGCCATCAT
GAGGCACATCTTGGAGAGACCGGACCCTACCGACGTCTTCCAGAATAAGGCAAACGTGTGTTGGG
CCAAGGCTTTAGTGCCGGTGCTGAAGACCGCTGGCATAGACATGACCACTGAACAATGGAACACT
GTGGATTATTTTGAAACGGACAAAGCTCACTCAGCAGAGATAGTATTGAACCAACTATGCGTGAG
GTTCTTTGGACTCGATCTGGACTCCGGTCTATTTTCTGCACCCACTGTTCCGTTATCCATTAGGAAT
AATCACTGGGATAACTCCCCGTCGCCTAACATGTACGGGCTGAATAAAGAAGTGGTCCGTCAGCTC
TCTCGCAGGTACCCACAACTGCCTCGGGCAGTTGCCACTGGAAGAGTCTATGACATGAACACTGGT
ACACTGCGCAATTATGATCCGCGCATAAACCTAGTACCTGTAAACAGAAGACTGCCTCATGCTTTA
GTCCTCCACCATAATGAACACCCACAGAGTGACTTTTCTTCATTCGTCAGCAAATTGAAGGGCAGA
ACTGTCCTGGTGGTCGGGGAAAAGTTGTCCGTCCCAGGCAAAATGGTTGACTGGTTGTCAGACCGG
CCTGAGGCTACCTTCAGAGCTCGGCTGGATTTAGGCATCCCAGGTGATGTGCCCAAATATGACATA
ATATTTGTTAATGTGAGGACCCCATATAAATACCATCACTATCAGCAGTGTGAAGACCATGCCATT
AAGCTTAGCATGTTGACCAAGAAAGCTTGTCTGCATCTGAATCCCGGCGGAACCTGTGTCAGCATA
GGTTATGGTTACGCTGACAGGGCCAGCGAAAGCATCATTGGTGCTATAGCGCGGCAGTTCAAGTTT
TCCCGGGTATGCAAACCGAAATCCTCACTTGAAGAGACGGAAGTTCTGTTTGTATTCATTGGGTAC
GATCGCAAGGCCCGTACGCACAATCCTTACAAGCTTTCATCAACCTTGACCAACATTTATACAGGT
TCCAGACTCCACGAAGCCGGATGTGCACCCTCATATCATGTGGTGCGAGGGGATATTGCCACGGCC
ACCGAAGGAGTGATTATAAATGCTGCTAACAGCAAAGGACAACCTGGCGGAGGGGTGTGCGGAGC
GCTGTATAAGAAATTCCCGGAAAGCTTCGATTTACAGCCGATCGAAGTAGGAAAAGCGCGACTGG
TCAAAGGTGCAGCTAAACATATCATTCATGCCGTAGGACCAAACTTCAACAAAGTTTCGGAGGTTG
AAGGTGACAAACAGTTGGCAGAGGCTTATGAGTCCATCGCTAAGATTGTCAACGATAACAATTAC
AAGTCAGTAGCGATTCCACTGTTGTCCACCGGCATCTTTTCCGGGAACAAAGATCGACTAACCCAA
TCATTGAACCATTTGCTGACAGCTTTAGACACCACTGATGCAGATGTAGCCATATACTGCAGGGAC
AAGAAATGGGAAATGACTCTCAAGGAAGCAGTGGCTAGGAGAGAAGCAGTGGAGGAGATATGCA
TATCCGACGACTCTTCAGTGACAGAACCTGATGCAGAGCTGGTGAGGGTGCATCCGAAGAGTTCTT
TGGCTGGAAGGAAGGGCTACAGCACAAGCGATGGCAAAACTTTCTCATATTTGGAAGGGACCAAG
TTTCACCAGGCGGCCAAGGATATAGCAGAAATTAATGCCATGTGGCCCGTTGCAACGGAGGCCAA
TGAGCAGGTATGCATGTATATCCTCGGAGAAAGCATGAGCAGTATTAGGTCGAAATGCCCCGTCG
AAGAGTCGGAAGCCTCCACACCACCTAGCACGCTGCCTTGCTTGTGCATCCATGCCATGACTCCAG
AAAGAGTACAGCGCCTAAAAGCCTCACGTCCAGAACAAATTACTGTGTGCTCATCCTTTCCATTGC
CGAAGTATAGAATCACTGGTGTGCAGAAGATCCAATGCTCCCAGCCTATATTGTTCTCACCGAAAG
TGCCTGCGTATATTCATCCAAGGAAGTATCTCGTGGAAACACCACCGGTAGACGAGACTCCGGAGC
CATCGGCAGAGAACCAATCCACAGAGGGGACACCTGAACAACCACCACTTATAACCGAGGATGAG
ACCAGGACTAGAACGCCTGAGCCGATCATCATCGAAGAGGAAGAAGAGGATAGCATAAGTTTGCT
GTCAGATGGCCCGACCCACCAGGTGCTGCAAGTCGAGGCAGACATTCACGGGCCGCCCTCTGTATC
TAGCTCATCCTGGTCCATTCCTCATGCATCCGACTTTGATGTGGACAGTTTATCCATACTTGACACC
CTGGAGGGAGCTAGCGTGACCAGCGGGGCAACGTCAGCCGAGACTAACTCTTACTTCGCAAAGAG
TATGGAGTTTCTGGCGCGACCGGTGCCTGCGCCTCGAACAGTATTCAGGAACCCTCCACATCCCGC
TCCGCGCACAAGAACACCGTCACTTGCACCCAGCAGGGCCTGCTCGAGAACCAGCCTAGTTTCCAC
CCCGCCAGGCGTGAATAGGGTGATCACTAGAGAGGAGCTCGAGGCGCTTACCCCGTCACGCACTC
CTAGCAGGTCGGTCTCGAGAACCAGCCTGGTCTCCAACCCGCCAGGCGTAAATAGGGTGATTACA
AGAGAGGAGTTTGAGGCGTTCGTAGCACAACAACAATGACGGTTTGATGCGGGTGCATACATCTTT
TCCTCCGACACCGGTCAAGGGCATTTACAACAAAAATCAGTAAGGCAAACGGTGCTATCCGAAGT
GGTGTTGGAGAGGACCGAATTGGAGATTTCGTATGCCCCGCGCCTCGACCAAGAAAAAGAAGAAT
TACTACGCAAGAAATTACAGTTAAATCCCACACCTGCTAACAGAAGCAGATACCAGTCCAGGAAG
GTGGAGAACATGAAAGCCATAACAGCTAGACGTATTCTGCAAGGCCTAGGGCATTATTTGAAGGC
AGAAGGAAAAGTGGAGTGCTACCGAACCCTGCATCCTGTTCCTTTGTATTCATCTAGTGTGAACCG
TGCCTTTTCAAGCCCCAAGGTCGCAGTGGAAGCCTGTAACGCCATGTTGAAAGAGAACTTTCCGAC
TGTGGCTTCTTACTGTATTATTCCAGAGTACGATGCCTATTTGGACATGGTTGACGGAGCTTCATGC
TGCTTAGACACTGCCAGTTTTTGCCCTGCAAAGCTGCGCAGCTTTCCAAAGAAACACTCCTATTTGG
AACCCACAATACGATCGGCAGTGCCTTCAGCGATCCAGAACACGCTCCAGAACGTCCTGGCAGCT
GCCACAAAAAGAAATTGCAATGTCACGCAAATGAGAGAATTGCCCGTATTGGATTCGGCGGCCTTT
AATGTGGAATGCTTCAAGAAATATGCGTGTAATAATGAATATTGGGAAACGTTTAAAGAAAACCC
CATCAGGCTTACTGAAGAAAACGTGGTAAATTACATTACCAAATTAAAAGGACCAAAAGCTGCTG
CTCTTTTTGCGAAGACACATAATTTGAATATGTTGCAGGACATACCAATGGACAGGTTTGTAATGG
ACTTAAAGAGAGACGTGAAAGTGACTCCAGGAACAAAACATACTGAAGAACGGCCCAAGGTACA
GGTGATCCAGGCTGCCGATCCGCTAGCAACAGCGTATCTGTGCGGAATCCACCGAGAGCTGGTTAG
GAGATTAAATGCGGTCCTGCTTCCGAACATTCATACACTGTTTGATATGTCGGCTGAAGACTTTGA
CGCTATTATAGCCGAGCACTTCCAGCCTGGGGATTGTGTTCTGGAAACTGACATCGCGTCGTTTGA
TAAAAGTGAGGACGACGCCATGGCTCTGACCGCGTTAATGATTCTGGAAGACTTAGGTGTGGACG
CAGAGCTGTTGACGCTGATTGAGGCGGCTTTCGGCGAAATTTCATCAATACATTTGCCCACTAAAA
CTAAATTTAAATTCGGAGCCATGATGAAATCTGGAATGTTCCTCACACTGTTTGTGAACACAGTCA
TTAACATTGTAATCGCAAGCAGAGTGTTGAGAGAACGGCTAACCGGATCACCATGTGCAGCATTCA
TTGGAGATGACAATATCGTGAAAGGAGTCAAATCGGACAAATTAATGGCAGACAGGTGCGCCACC
TGGTTGAATATGGAAGTCAAGATTATAGATGCTGTGGTGGGCGAGAAAGCGCCTTATTTCTGTGGA
GGGTTTATTTTGTGTGACTCCGTGACCGGCACAGCGTGCCGTGTGGCAGACCCCCTAAAAAGGCTG
TTTAAGCTTGGCAAACCTCTGGCAGCAGACGATGAACATGATGATGACAGGAGAAGGGCATTGCA
TGAAGAGTCAACACGCTGGAACCGAGTGGGTATTCTTTCAGAGCTGTGCAAGGCAGTAGAATCAA
GGTATGAAACCGTAGGAACTTCCATCATAGTTATGGCCATGACTACTCTAGCTAGCAGTGTTAAAT
CATTCAGCTACCTGAGAGGGGCCCCTATAACTCTCTACGGCTAACCTGAATGGACTACGACGTATC
ACGCCCAAACATTTACAGCCGCGGTGTCAAAAACCGCGTGGACGTGGTTAACATCCCTGCTGGGA
GGATCAGCCGTAATTATTATAATTGGCTTGGTGCTGGCTACTATTGTGGCCATGTACGTGCTGACCA
ACCAGAAACATAATTGAATACAGCAGCAATTGGCAAGCTGCTTACATAGAACTCGCGGCGATTGG
CATGCCGCCTTAAAATTTTTATTTTATTTTTCTTTTCTTTTCCGAATCGGATTTTGTTTTTAATATTTC
AAAAAAAAAAAAAAAAAAAAAAAAAAAAAAAAAAAAAAAAAAAAAAAAAAAAAAAAAAAAAA
AAAAAAAAAAAAAAAAAA
VEE-UbAAY (SEQ ID NO: 14); VEE delivery vector with MHC class I mouse tumor epitopes SIINFEKL (SEQ
ID NO: 129) and AH1-A5 inserted
VEE-Luciferase (SEQ ID NO: 15); VEE delivery vector with luciferase gene inserted at 7545
ubiquitin (SEQ ID NO: 38) >UbG76 0-228
Ubiquitin A76 (SEQ ID NO: 39) >UbA76 0-228
HLA-A2 (MHC class I) signal peptide (SEQ ID NO: 40) >MHC SignalPep 0-78
HLA-A2 (MHC class I) Trans Membrane domain (SEQ ID NO: 41) >HLA A2 TM Domain 0-201
IgK Leader Seq (SEQ ID NO: 42) >IgK Leader Seq 0-60
Human DC-Lamp (SEQ ID NO: 43) >HumanDCLAMP 0-3178
Mouse LAMP1 (SEQ ID NO: 44) >MouseLamp1 0-1858
Human Lamp1 cDNA (SEQ ID NO: 45) >Human Lamp1 0-2339
Tetanus toxoid nulceic acid sequence (SEQ ID NO: 46)
Tetanus toxoid amino acid sequence (SEQ ID NO: 47)
PADRE nulceotide sequence (SEQ ID NO: 48)
PADRE amino acid sequence (SEQ ID NO: 49)
WPRE (SEQ ID NO: 50) >WPRE 0-593
IRES (SEQ ID NO: 51) >eGFP_IRES_SEAP_Insert 1746-2335
GFP (SEQ ID NO: 52)
SEAP (SEQ ID NO: 53)
Firefly Luciferase (SEQ ID NO: 54)
FMDV 2A (SEQ ID NO: 55)
GPGPG linker (SEQ ID NO: 56)
chAd68-Empty-E4deleted (SEQ ID NO: 75): AC_000011.1 with E1 (nt 577 to 3403), E3 (nt 27,125- 31,825),
and E4 region (nt 34,916 to 35,642) sequences deleted and the corresponding ATCC VR-594 (Independently
sequenced Full-Length VR-594 C68 SEQ ID NO: 10) nucleotides substituted at five positions
NC_045512.2 Severe acute respiratory syndrome coronavirus 2 isolate Wuhan-Hu-1, complete genome (SEQ ID
NO: 76)
SAM in vitro transcription template DNA (SEQ ID NO: 77); VEE genome with nucleotides 7544-11175 deleted,
plus minimal 5′ T7-promoter
TAATACGACTCACTATAATGggcggcgcatgagagaagcccagaccaattacctacccaaaATGGagaaagttcacgttgacatcgaggaa
gacagcccattcctcagagctttgcagcggagcttcccgcagtttgaggtagaagccaagcaggtcactgataatgaccatgctaatgccagagcgttttcgcatct
ggcttcaaaactgatcgaaacggaggtggacccatccgacacgatccttgacattggaagtgcgcccgcccgcagaatgtattctaagcacaagtatcattgtatct
gtccgatgagatgtgcggaagatccggacagattgtataagtatgcaactaagctgaagaaaaactgtaaggaaataactgataaggaattggacaagaaaatgaa
ggagctcgccgccgtcatgagcgaccctgacctggaaactgagactatgtgcctccacgacgacgagtcgtgtcgctacgaagggcaagtcgctgtttaccagga
tgtatacgcggttgacggaccgacaagtctctatcaccaagccaataagggagttagagtcgcctactggataggctttgacaccaccccttttatgtttaagaacttg
gctggagcatatccatcatactctaccaactgggccgacgaaaccgtgttaacggctcgtaacataggcctatgcagctctgacgttatggagcggtcacgtagag
ggatgtccattcttagaaagaagtatttgaaaccatccaacaatgttctattctctgttggctcgaccatctaccacgagaagagggacttactgaggagctggcacct
gccgtctgtatttcacttacgtggcaagcaaaattacacatgtcggtgtgagactatagttagttgcgacgggtacgtcgttaaaagaatagctatcagtccaggcctgt
atgggaagccttcaggctatgctgctacgatgcaccgcgagggattcttgtgctgcaaagtgacagacacattgaacggggagagggtctcttttcccgtgtgcacg
tatgtgccagctacattgtgtgaccaaatgactggcatactggcaacagatgtcagtgcggacgacgcgcaaaaactgctggttgggctcaaccagcgtatagtcgt
caacggtcgcacccagagaaacaccaataccatgaaaaattaccttttgcccgtagtggcccaggcatttgctaggtgggcaaaggaatataaggaagatcaaga
agatgaaaggccactaggactacgagatagacagttagtcatggggtgttgttgggcttttagaaggcacaagataacatctatttataagcgcccggatacccaaa
ccatcatcaaagtgaacagcgatttccactcattcgtgctgcccaggataggcagtaacacattggagatcgggctgagaacaagaatcaggaaaatgttagagga
gcacaaggagccgtcacctctcattaccgccgaggacgtacaagaagctaagtgcgcagccgatgaggctaaggaggtgcgtgaagccgaggagttgcgcgc
agctctaccacctttggcagctgatgttgaggagcccactctggaagccgatgtcgacttgatgttacaagaggctggggccggctcagtggagacacctcgtggc
ttgataaaggttaccagctacgctggcgaggacaagatcggctcttacgctgtgctttctccgcaggctgtactcaagagtgaaaaattatcttgcatccaccctctcg
ctgaacaagtcatagtgataacacactctggccgaaaagggcgttatgccgtggaaccataccatggtaaagtagtggtgccagagggacatgcaatacccgtcc
aggactttcaagctctgagtgaaagtgccaccattgtgtacaacgaacgtgagttcgtaaacaggtacctgcaccatattgccacacatggaggagcgctgaacact
gatgaagaatattacaaaactgtcaagcccagcgagcacgacggcgaatacctgtacgacatcgacaggaaacagtgcgtcaagaaagaactagtcactgggct
agggctcacaggcgagctggtggatcctcccttccatgaattcgcctacgagagtctgagaacacgaccagccgctccttaccaagtaccaaccataggggtgtat
ggcgtgccaggatcaggcaagtctggcatcattaaaagcgcagtcaccaaaaaagatctagtggtgagcgccaagaaagaaaactgtgcagaaattataaggga
cgtcaagaaaatgaaagggctggacgtcaatgccagaactgtggactcagtgctcttgaatggatgcaaacaccccgtagagaccctgtatattgacgaagcttttg
cttgtcatgcaggtactctcagagcgctcatagccattataagacctaaaaaggcagtgctctgcggggatcccaaacagtgcggtttttttaacatgatgtgcctgaa
agtgcattttaaccacgagatttgcacacaagtcttccacaaaagcatctctcgccgttgcactaaatctgtgacttcggtcgtctcaaccttgttttacgacaaaaaaat
gagaacgacgaatccgaaagagactaagattgtgattgacactaccggcagtaccaaacctaagcaggacgatctcattctcacttgtttcagagggtgggtgaag
cagttgcaaatagattacaaaggcaacgaaataatgacggcagctgcctctcaagggctgacccgtaaaggtgtgtatgccgttcggtacaaggtgaatgaaaatc
ctctgtacgcacccacctcagaacatgtgaacgtcctactgacccgcacggaggaccgcatcgtgtggaaaacactagccggcgacccatggataaaaacactg
actgccaagtaccctgggaatttcactgccacgatagaggagtggcaagcagagcatgatgccatcatgaggcacatcttggagagaccggaccctaccgacgtc
ttccagaataaggcaaacgtgtgttgggccaaggctttagtgccggtgctgaagaccgctggcatagacatgaccactgaacaatggaacactgtggattattttga
aacggacaaagctcactcagcagagatagtattgaaccaactatgcgtgaggttctttggactcgatctggactccggtctattttctgcacccactgttccgttatccat
taggaataatcactgggataactccccgtcgcctaacatgtacgggctgaataaagaagtggtccgtcagctctctcgcaggtacccacaactgcctcgggcagttg
ccactggaagagtctatgacatgaacactggtacactgcgcaattatgatccgcgcataaacctagtacctgtaaacagaagactgcctcatgctttagtcctccacc
ataatgaacacccacagagtgacttttcttcattcgtcagcaaattgaagggcagaactgtcctggtggtcggggaaaagttgtccgtcccaggcaaaatggttgact
ggttgtcagaccggcctgaggctaccttcagagctcggctggatttaggcatcccaggtgatgtgcccaaatatgacataatatttgttaatgtgaggaccccatataa
ataccatcactatcagcagtgtgaagaccatgccattaagcttagcatgttgaccaagaaagcttgtctgcatctgaatcccggcggaacctgtgtcagcataggttat
ggttacgctgacagggccagcgaaagcatcattggtgctatagcgcggcagttcaagttttcccgggtatgcaaaccgaaatcctcacttgaagagacggaagttct
gtttgtattcattgggtacgatcgcaaggcccgtacgcacaatccttacaagctttcatcaaccttgaccaacatttatacaggttccagactccacgaagccggatgtg
caccctcatatcatgtggtgcgaggggatattgccacggccaccgaaggagtgattataaatgctgctaacagcaaaggacaacctggcggaggggtgtgcgga
gcgctgtataagaaattcccggaaagcttcgatttacagccgatcgaagtaggaaaagcgcgactggtcaaaggtgcagctaaacatatcattcatgccgtaggac
caaacttcaacaaagtttcggaggttgaaggtgacaaacagttggcagaggcttatgagtccatcgctaagattgtcaacgataacaattacaagtcagtagcgattc
cactgttgtccaccggcatcttttccgggaacaaagatcgactaacccaatcattgaaccatttgctgacagctttagacaccactgatgcagatgtagccatatactg
cagggacaagaaatgggaaatgactctcaaggaagcagtggctaggagagaagcagtggaggagatatgcatatccgacgactcttcagtgacagaacctgatg
cagagctggtgagggtgcatccgaagagttctttggctggaaggaagggctacagcacaagcgatggcaaaactttctcatatttggaagggaccaagtttcacca
ggcggccaaggatatagcagaaattaatgccatgtggcccgttgcaacggaggccaatgagcaggtatgcatgtatatcctcggagaaagcatgagcagtattag
gtcgaaatgccccgtcgaagagtcggaagcctccacaccacctagcacgctgccttgcttgtgcatccatgccatgactccagaaagagtacagcgcctaaaagc
ctcacgtccagaacaaattactgtgtgctcatcctttccattgccgaagtatagaatcactggtgtgcagaagatccaatgctcccagcctatattgttctcaccgaaagt
gcctgcgtatattcatccaaggaagtatctcgtggaaacaccaccggtagacgagactccggagccatcggcagagaaccaatccacagaggggacacctgaac
aaccaccacttataaccgaggatgagaccaggactagaacgcctgagccgatcatcatcgaagaggaagaagaggatagcataagtttgctgtcagatggcccg
acccaccaggtgctgcaagtcgaggcagacattcacgggccgccctctgtatctagctcatcctggtccattcctcatgcatccgactttgatgtggacagtttatcca
tacttgacaccctggagggagctagcgtgaccagcggggcaacgtcagccgagactaactcttacttcgcaaagagtatggagtttctggcgcgaccggtgcctg
cgcctcgaacagtattcaggaaccctccacatcccgctccgcgcacaagaacaccgtcacttgcacccagcagggcctgctcgagaaccagcctagtttccaccc
cgccaggcgtgaatagggtgatcactagagaggagctcgaggcgcttaccccgtcacgcactcctagcaggtcggtctcgagaaccagcctggtctccaacccg
ccaggcgtaaatagggtgattacaagagaggagtttgaggcgttcgtagcacaacaacaatgacggtttgatgcgggtgcatacatcttttcctccgacaccggtca
agggcatttacaacaaaaatcagtaaggcaaacggtgctatccgaagtggtgttggagaggaccgaattggagatttcgtatgccccgcgcctcgaccaagaaaa
agaagaattactacgcaagaaattacagttaaatcccacacctgctaacagaagcagataccagtccaggaaggtggagaacatgaaagccataacagctagacg
tattctgcaaggcctagggcattatttgaaggcagaaggaaaagtggagtgctaccgaaccctgcatcctgttcctttgtattcatctagtgtgaaccgtgccttttcaa
gccccaaggtcgcagtggaagcctgtaacgccatgttgaaagagaactttccgactgtggcttcttactgtattattccagagtacgatgcctatttggacatggttgac
ggagcttcatgctgcttagacactgccagtttttgccctgcaaagctgcgcagctttccaaagaaacactcctatttggaacccacaatacgatcggcagtgccttcag
cgatccagaacacgctccagaacgtcctggcagctgccacaaaaagaaattgcaatgtcacgcaaatgagagaattgcccgtattggattcggcggcctttaatgt
ggaatgcttcaagaaatatgcgtgtaataatgaatattgggaaacgtttaaagaaaaccccatcaggcttactgaagaaaacgtggtaaattacattaccaaattaaaa
ggaccaaaagctgctgctctttttgcgaagacacataatttgaatatgttgcaggacataccaatggacaggtttgtaatggacttaaagagagacgtgaaagtgactc
caggaacaaaacatactgaagaacggcccaaggtacaggtgatccaggctgccgatccgctagcaacagcgtatctgtgcggaatccaccgagagctggttagg
agattaaatgcggtcctgcttccgaacattcatacactgtttgatatgtcggctgaagactttgacgctattatagccgagcacttccagcctggggattgtgttctggaa
actgacatcgcgtcgtttgataaaagtgaggacgacgccatggctctgaccgcgttaatgattctggaagacttaggtgtggacgcagagctgttgacgctgattga
ggcggctttcggcgaaatttcatcaatacatttgcccactaaaactaaatttaaattcggagccatgatgaaatctggaatgttcctcacactgtttgtgaacacagtcatt
aacattgtaatcgcaagcagagtgttgagagaacggctaaccggatcaccatgtgcagcattcattggagatgacaatatcgtgaaaggagtcaaatcggacaaatt
aatggcagacaggtgcgccacctggttgaatatggaagtcaagattatagatgctgtggtgggcgagaaagcgccttatttctgtggagggtttattttgtgtgactcc
gtgaccggcacagcgtgccgtgtggcagaccccctaaaaaggctgtttaagcttggcaaacctctggcagcagacgatgaacatgatgatgacaggagaagggc
attgcatgaagagtcaacacgctggaaccgagtgggtattctttcagagctgtgcaaggcagtagaatcaaggtatgaaaccgtaggaacttccatcatagttatggc
catgactactctagctagcagtgttaaatcattcagctacctgagaggggcccctataactctctacggcTAAcctgaatggactacgacgTatcacgcccaaac
atttacagccgcggtgtcaaaaaccgcgtggacgtggttaacatccctgctgggaggatcagccgtaattattataattggcttggtgctggctactattgtggccatgt
acgtgctgaccaaccagaaacataattgaatacagcagcaattggcaagctgcttacatagaactcgcggcgattggcatgccgccttaaaatttttattttattttttctt
ttcttttccgaatcggattttgtttttaatatttcAAAAAAAAAAAAAAAAAAAAAAAAAAAAAAAAAAAAAAAAAAAAA
AAAAAAAAAAAAAAAAAAAAAAAAAAAAAAAAAAA
SV40 mini-intron (SEQ ID NO: 88)
CMV-CTSpike-FurinMT2P-T2A-E-T2A-M-SV40-CMV-EPE-BGH Nucleotide sequence
(SEQ ID NO: 109)
VOC 202012/01 B.1.1.7 UK Spike variant Amino Acid Sequence (SEQ ID NO: 110)
MFVFLVLLPLVSSQCVNLTTRTQLPPAYTNSFTRGVYYPDKVFRSSVLHSTQDLFLPF
FSNVTWFHAISGTNGTKRFDNPVLPFNDGVYFASTEKSNIIRGWIFGTTLDSKTQSLLI
VNNATNVVIKVCEFQFCNDPFLGVYHKNNKSWMESEFRVYSSANNCTFEYVSQPFL
MDLEGKQGNFKNLREFVFKNIDGYFKIYSKHTPINLVRDLPQGFSALEPLVDLPIGINI
TRFQTLLALHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSFVIRGDEV
RQIAPGQTGKIADYNYKLPDDFTGCVIAWNSNNLDSKVGGNYNYLYRLFRKSNLKPF
ERDISTEIYQAGSTPCNGVEGENCYFPLQSYGFQPTYGVGYQPYRVVVLSFELLHAPA
TVCGPKKSTNLVKNKCVNFNFNGLTGTGVLTESNKKFLPFQQFGRDIDDTTDAVRDP
QTLEILDITPCSFGGVSVITPGTNTSNQVAVLYQGVNCTEVPVAIHADQLTPTWRVYS
TGSNVFQTRAGCLIGAEHVNNSYECDIPIGAGICASYQTQTNSHGSASSVASQSIIAYT
MSLGAENSVAYSNNSIAIPINFTISVTTEILPVSMTKTSVDCTMYICGDSTECSNLLLQY
GSFCTQLNRALTGIA VEQDKNTQEVFAQVKQIYKTPPIKDFGGFNFSQILPDPSKPSKR
SFIEDLLFNKVTLADAGFIKQYGDCLGDIAARDLICAQKFNGLTVLPPLLTDEMIAQY
TSALLAGTITSGWTFGAGAALQIPFAMQMAYRENGIGVTQNVLYENQKLIANQFNSA
IGKIQDSLSSTASALGKLQDVVNQNAQALNTLVKQLSSNFGAISSVLNDILARLDPPE
AEVQIDRLITGRLQSLQTYVTQQLIRAAEIRASANLAATKMSECVLGQSKRVDFCGK
GYHLMSFPQSAPHGVVFLHVTYVPAQEKNFTTAPAICHDGKAHFPREGVFVSNGTH
WFVTQRNFYEPQIITTHNTFVSGNCDVVIGIVNNTVYDPLQPELDSFKEELDKYFKNH
TSPDVDLGDISGINASVVNIQKEIDRLNEVAKNLNESLIDLQELGKYEQYIKWPWYIW
LGFIAGLIAIVMVTIMLCCMTSCCSCLKGCCSCGSCCKFDEDDSEPVLKGVKLHYT*
CMV-B117Spike-FurinMT2P-SV40-CMV-EPE-BGH (SEQ ID NO: 111)
B.1.351Spike-FurinMt Amino Acid sequence (South African Spike Variant) (SEQ ID
NO: 112)
MFVFLVLLPLVSSQCVNFTTRTQLPPAYTNSFTRGVYYPDKVFRSSVLHSTQDLFLPFFSNVTWFHAIHV
SGTNGTKRFANPVLPFNDGVYFASTEKSNIIRGWIFGTTLDSKTQSLLIVNNATNVVIKVCEFQFCNDPF
LGVYYHKNNKSWMESEFRVYSSANNCTFEYVSQPFLMDLEGKQGNFKNLREFVFKNIDGYFKIYSKHT
PINLVRGLPQGFSALEPLVDLPIGINITRFQTLHISYLTPGDSSSGWTAGAAAYYVGYLQPRTFLLKYNEN
GTITDAVDCALDPLSETKCTLKSFTVEKGIYQTSNFRVQPTESIVRFPNITNLCPFGEVENATRFASVYAW
NRKRISNCVADYSVLYNSASFSTFKCYGVSPTKLNDLCFTNVYADSFVIRGDEVRQIAPGQTGNIADYN
YKLPDDFTGCVIAWNSNNLDSKVGGNYNYLYRLFRKSNLKPFERDISTEIYQAGSTPCNGVKGENCYFP
LQSYGFQPTYGVGYQPYRVVVLSFELLHAPATVCGPKKSTNLVKNKCVNFNFNGLTGTGVLTESNKKF
LPFQQFGRDIADTTDAVRDPQTLEILDITPCSFGGVSVITPGTNTSNQVAVLYQGVNCTEVPVAIHADQL
TPTWRVYSTGSNVFQTRAGCLIGAEHVNNSYECDIPIGAGICASYQTQTNSPGSASSVASQSIIAYTMSL
GVENSVAYSNNSIAIPTNFTISVTTEILPVSMTKTSVDCTMYICGDSTECSNLLLQYGSFCTQLNRALTGI
AVEQDKNTQEVFAQVKQIYKTPPIKDFGGFNFSQILPDPSKPSKRSFIEDLLFNKVTLADAGFIKQYGDC
LGDIAARDLICAQKFNGLTVLPPLLTDEMIAQYTSALLAGTITSGWTFGAGAALQIPFAMQMAYRENGI
GVTQNVLYENQKLIANQFNSAIGKIQDSLSSTASALGKLQDVVNQNAQALNTLVKQLSSNFGAISSVLN
DILSRLDPPEAEVQIDRLITGRLQSLQTYVTQQLIRAAEIRASANLAATKMSECVLGQSKRVDFCGKGYH
LMSFPQSAPHGVVFLHVTYVPAQEKNFTTAPAICHDGKAHFPREGVFVSNGTHWFVTQRNFYEPQIITT
DNTFVSGNCDVVIGIVNNTVYDPLQPELDSFKEELDKYFKNHTSPDVDLGDISGINASVVNIQKEIDRLN
EVAKNLNESLIDLQELGKYEQYIKWPWYIWLGFIAGLIAIVMVTIMLCCMTSCCSCLKGCCSCGSCCKF
DEDDSEPVLKGVKLHYT
CMV-B.1351Spike-FurinMt-2P-CMV-TCE5-BGH (SEQ ID NO: 113); B.1.351 Spike
sequence based on CT1 sequence optimization with relevant variant mutations replaced with
COOL codons

### REFERENCES

1. Desrichard, A., Snyder, A. & Chan, T. A. Cancer Neoantigens and Applications for Immunotherapy. Clin. Cancer Res. Off. J. Am. Assoc. Cancer Res. (2015). doi: 10.1158/1078-0432.CCR-14-3175
2. Schumacher, T. N. & Schreiber, R. D. Neoantigens in cancer immunotherapy. Science 348, 69-74 (2015).
3. Gubin, M. M., Artyomov, M. N., Mardis, E. R. & Schreiber, R. D. Tumor neoantigens: building a framework for personalized cancer immunotherapy. J. Clin. Invest. 125, 3413-3421 (2015).
4. Rizvi, N. A. et al. Cancer immunology. Mutational landscape determines sensitivity to PD-1 blockade in non-small cell lung cancer. Science 348, 124-128 (2015).
5. Snyder, A. et al. Genetic basis for clinical response to CTLA-4 blockade in melanoma. N. Engl. J. Med. 371, 2189-2199 (2014).
6. Carreno, B. M. et al. Cancer immunotherapy. A dendritic cell vaccine increases the breadth and diversity of melanoma neoantigen-specific T cells. Science 348, 803-808 (2015).
7. Tran, E. et al. Cancer immunotherapy based on mutation-specific CD4+ T cells in a patient with epithelial cancer. Science 344, 641-645 (2014).
8. Hacohen, N. & Wu, C. J.-Y. United States Patent Application: 20110293637-COMPOSITIONS AND METHODS OF IDENTIFYING TUMOR SPECIFIC NEOANTIGENS. (A1). at <appft1.uspto.gov/netacgi/nph-Parser?Sect1=PTO1&Sect2=HITOFF&d=PG01&p=1&u=/netahtml/PTO/srchnum.html&r=1 &f=G&1=50&s1=20110293637.PGNR.>
9. Lundegaard, C., Hoof, I., Lund, O. & Nielsen, M. State of the art and challenges in sequence based T-cell epitope prediction. Immunome Res. 6 Suppl 2, S3 (2010).
10. Yadav, M. et al. Predicting immunogenic tumour mutations by combining mass spectrometry and exome sequencing. Nature 515, 572-576 (2014).
11. Bassani-Sternberg, M., Pletscher-Frankild, S., Jensen, L. J. & Mann, M. Mass spectrometry of human leukocyte antigen class I peptidomes reveals strong effects of protein abundance and turnover on antigen presentation. Mol. Cell. Proteomics MCP 14, 658-673 (2015).
12. Van Allen, E. M. et al. Genomic correlates of response to CTLA-4 blockade in metastatic melanoma. Science 350, 207-211 (2015).
13. Yoshida, K. & Ogawa, S. Splicing factor mutations and cancer. Wiley Interdiscip. Rev. RNA 5, 445-459 (2014).
14. Cancer Genome Atlas Research Network. Comprehensive molecular profiling of lung adenocarcinoma. Nature 511, 543-550 (2014).
15. Rajasagi, M. et al. Systematic identification of personal tumor-specific neoantigens in chronic lymphocytic leukemia. Blood 124, 453-462 (2014).
16. Downing, S. R. et al. U.S. patent application Ser. No. 01/202,08706-OPTIMIZATION OF MULTIGENE ANALYSIS OF TUMOR SAMPLES. (A1). at <appft 1.uspto.gov/nctacgi/nph-Parser?Sect1=PTO1&Sect2=HITOFF&d=PG01&p=1&u=/nctahtml/PTO/srchnum.html&r=1&f=G&1=50&s1=20120208706.PGNR.>
17. Target Capture for NextGen Sequencing—IDT. at <www.idtdna.com/pages/products/nextgen/target-capture>
18. Shukla, S. A. et al. Comprehensive analysis of cancer-associated somatic mutations in class I HLA genes. Nat. Biotechnol. 33, 1152-1158 (2015).
19. Cieslik, M. et al. The use of exome capture RNA-seq for highly degraded RNA with application to clinical cancer sequencing. Genome Res. 25, 1372-1381 (2015).
20. Bodini, M. et al. The hidden genomic landscape of acute myeloid leukemia: subclonal structure revealed by undetected mutations. Blood 125, 600-605 (2015).
21. Saunders, C. T. et al. Strelka: accurate somatic small-variant calling from sequenced tumor-normal sample pairs. Bioinforma. Oxf. Engl. 28, 1811-1817 (2012).
22. Cibulskis, K. et al. Sensitive detection of somatic point mutations in impure and heterogeneous cancer samples. Nat. Biotechnol. 31, 213-219 (2013).
23. Wilkerson, M. D. et al. Integrated RNA and DNA sequencing improves mutation detection in low purity tumors. Nucleic Acids Res. 42, c107 (2014).
24. Mose, L. E., Wilkerson, M. D., Hayes, D. N., Perou, C. M. & Parker, J. S. ABRA: improved coding indel detection via assembly-based realignment. Bioinforma. Oxf. Engl. 30, 2813-2815 (2014).
25. Ye, K., Schulz, M. H., Long, Q., Apweiler, R. & Ning, Z. Pindel: a pattern growth approach to detect break points of large deletions and medium sized insertions from paired-end short reads. Bioinforma. Oxf. Engl. 25, 2865-2871 (2009).
26. Lam, H. Y. K. et al. Nucleotide-resolution analysis of structural variants using BreakSeq and a breakpoint library. Nat. Biotechnol. 28, 47-55 (2010).
27. Frampton, G. M. et al. Development and validation of a clinical cancer genomic profiling test based on massively parallel DNA sequencing. Nat. Biotechnol. 31, 1023-1031 (2013).
28. Boegel, S. et al. HLA typing from RNA-Seq sequence reads. Genome Med. 4, 102 (2012).
29. Liu, C. et al. ATHLATES: accurate typing of human leukocyte antigen through exome sequencing. Nucleic Acids Res. 41, c142 (2013).
30. Mayor, N. P. et al. HLA Typing for the Next Generation. PloS One 10, c0127153 (2015).
31. Roy, C. K., Olson, S., Graveley, B. R., Zamore, P. D. & Moore, M. J. Assessing long-distance RNA sequence connectivity via RNA-templated DNA-DNA ligation. eLife 4, (2015).
32. Song, L. & Florea, L. CLASS: constrained transcript assembly of RNA-seq reads. BMC Bioinformatics 14 Suppl 5, S14 (2013).
33. Maretty, L., Sibbesen, J. A. & Krogh, A. Bayesian transcriptome assembly. Genome Biol. 15, 501 (2014).
34. Pertea, M. et al. StringTie enables improved reconstruction of a transcriptome from RNA-seq reads. Nat. Biotechnol. 33, 290-295 (2015).
35. Roberts, A., Pimentel, H., Trapnell, C. & Pachter, L. Identification of novel transcripts in annotated genomes using RNA-Seq. Bioinforma. Oxf. Engl. (2011). doi: 10.1093/bioinformatics/btr355
36. Vitting-Seerup, K., Porse, B. T., Sandelin, A. & Waage, J. spliceR: an R package for classification of alternative splicing and prediction of coding potential from RNA-seq data. BMC Bioinformatics 15, 81 (2014).
37. Rivas, M. A. et al. Human genomics. Effect of predicted protein-truncating genetic variants on the human transcriptome. Science 348, 666-669 (2015).
38. Skelly, D. A., Johansson, M., Madeoy, J., Wakefield, J. & Akey, J. M. A powerful and flexible statistical framework for testing hypotheses of allele-specific gene expression from RNA-seq data. Genome Res. 21, 1728-1737 (2011).
39. Anders, S., Pyl, P. T. & Huber, W. HTSeq—a Python framework to work with high-throughput sequencing data. Bioinforma. Oxf. Engl. 31, 166-169 (2015).
40. Furney, S. J. et al. SF3B1 mutations are associated with alternative splicing in uveal melanoma. Cancer Discov. (2013). doi: 10.1158/2159-8290.CD-13-0330
41. Zhou, Q. et al. A chemical genetics approach for the functional assessment of novel cancer genes. Cancer Res. (2015). doi: 10.1158/0008-5472.CAN-14-2930
42. Maguire, S. L. et al. SF3B1 mutations constitute a novel therapeutic target in breast cancer. J. Pathol. 235, 571-580 (2015).
43. Carithers, L. J. et al. A Novel Approach to High-Quality Postmortem Tissue Procurement: The GTEx Project. Biopreservation Biobanking 13, 311-319 (2015).
44. Xu, G. et al. RNA COMPASS: a dual approach for pathogen and host transcriptome analysis of RNA-seq datasets. PloS One 9, c89445 (2014).
45. Andreatta, M. & Nielsen, M. Gapped sequence alignment using artificial neural networks: application to the MHC class I system. Bioinforma. Oxf. Engl. (2015). doi: 10.1093/bioinformatics/btv639
46. Jørgensen, K. W., Rasmussen, M., Buus, S. & Nielsen, M. NetMHCstab-predicting stability of peptide-MHC-I complexes; impacts for cytotoxic T lymphocyte epitope discovery. Immunology 141, 18-26 (2014).
47. Larsen, M. V. et al. An integrative approach to CTL epitope prediction: a combined algorithm integrating MHC class I binding, TAP transport efficiency, and proteasomal cleavage predictions. Eur. J. Immunol. 35, 2295-2303 (2005).
48. Nielsen, M., Lundegaard, C., Lund, O. & Kesmir, C. The role of the proteasome in generating cytotoxic T-cell epitopes: insights obtained from improved predictions of protcasomal cleavage. Immunogenetics 57, 33-41 (2005).
49. Boisvert, F.-M. et al. A Quantitative Spatial Proteomics Analysis of Proteome Turnover in Human Cells. Mol. Cell. Proteomics 11, M111.011429-M111.011429 (2012).
50. Duan, F. et al. Genomic and bioinformatic profiling of mutational neoepitopes reveals new rules to predict anticancer immunogenicity. J. Exp. Med. 211, 2231-2248 (2014).
51. Janeway's Immunobiology: 9780815345312: Medicine & Health Science Books @ Amazon.com. at <www.amazon.com/Janeways-Immunobiology-Kenneth-Murphy/dp/0815345313>
52. Calis, J. J. A. et al. Properties of MHC Class I Presented Peptides That Enhance Immunogenicity. PLOS Comput. Biol. 9, e1003266 (2013).
53. Zhang, J. et al. Intratumor heterogeneity in localized lung adenocarcinomas delineated by multiregion sequencing. Science 346, 256-259 (2014)
54. Walter, M. J. et al. Clonal architecture of secondary acute myeloid leukemia. N. Engl. J. Med. 366, 1090-1098 (2012).
55. Hunt D F, Henderson R A, Shabanowitz J, Sakaguchi K, Michel H, Sevilir N, Cox A L, Appella E, Engelhard V H. Characterization of peptides bound to the class I MHC molecule HLA-A2.1 by mass spectrometry. Science 1992. 255: 1261-1263.
56. Zarling A L, Polefrone J M, Evans A M, Mikesh L M, Shabanowitz J, Lewis S T, Engelhard V H, Hunt D F. Identification of class I MHC-associated phosphopeptides as targets for cancer immunotherapy. Proc Natl Acad Sci USA. 2006 Oct. 3; 103(40): 14889-94.
57. Bassani-Sternberg M, Pletscher-Frankild S, Jensen L J, Mann M. Mass spectrometry of human leukocyte antigen class I peptidomes reveals strong effects of protein abundance and turnover on antigen presentation. Mol Cell Proteomics. 2015 March; 14(3):658-73. doi: 10.1074/mcp.M114.042812.
58. Abelin J G, Trantham P D, Penny S A, Patterson A M, Ward S T, Hildebrand W H, Cobbold M, Bai D L, Shabanowitz J, Hunt D F. Complementary IMAC enrichment methods for HLA-associated phosphopeptide identification by mass spectrometry. Nat Protoc. 2015 September; 10(9):1308-18. doi: 10.1038/nprot.2015.086. Epub 2015 Aug. 6
59. Barnstable C J, Bodmer W F, Brown G, Galfre G, Milstein C, Williams A F, Ziegler A. Production of monoclonal antibodies to group A erythrocytes, HLA and other human cell surface antigens-new tools for genetic analysis. Cell. 1978 May; 14(1):9-20.
60. Goldman J M, Hibbin J, Kearney L, Orchard K, Th'ng KH. HLA-DR monoclonal antibodies inhibit the proliferation of normal and chronic granulocytic leukaemia myeloid progenitor cells. Br J Haematol. 1982 November; 52(3):411-20.
61. Eng J K, Jahan T A, Hoopmann M R. Comet: an open-source M S/M S sequence database search tool. Proteomics. 2013 January; 13(1):22-4. doi: 10.1002/pmic.201200439. Epub 2012 Dec. 4.
62. Eng J K, Hoopmann M R, Jahan T A, Egertson J D, Noble W S, MacCoss M J. A deeper look into Comet—implementation and features. J Am Soc Mass Spectrom. 2015 November; 26(11): 1865-74. doi: 10.1007/s13361-015-1179-x. Epub 2015 Jun. 27.
63. Lukas Kall, Jesse Canterbury, Jason Weston, William Stafford Noble and Michael J. MacCoss. Semi-supervised learning for peptide identification from shotgun proteomics datasets. Nature Methods 4:923-925, November 2007
64. Lukas Käll, John D. Storey, Michael J. MacCoss and William Stafford Noble. Assigning confidence measures to peptides identified by tandem mass spectrometry. Journal of Proteome Research, 7(1):29-34, January 2008
65. Lukas Käll, John D. Storey and William Stafford Noble. Nonparametric estimation of posterior error probabilities associated with peptides identified by tandem mass spectrometry. Bioinformatics, 24(16):142-148, August 2008
66. Kinney R M, B J Johnson, V L Brown, D W Trent. Nucleotide Sequence of the 26 S mRNA of the Virulent Trinidad Donkey Strain of Venezuelan Equine Encephalitis Virus and Deduced Sequence of the Encoded Structural Proteins. Virology 152 (2), 400-413. 1986 Jul. 30.
67. Jill E Slansky, Frederique M Rattis, Lisa F Boyd, Tarek Fahmy, Elizabeth M Jaffee, Jonathan P Schneck, David H Margulies, Drew M Pardoll. Enhanced Antigen-Specific Antitumor Immunity with Altered Peptide Ligands that Stabilize the MHC-Peptide-TCR Complex. Immunity, Volume 13, Issue 4, 1 Oct. 2000, Pages 529-538.
68. A Y Huang, P H Gulden, A S Woods, M C Thomas, C D Tong, W Wang, V H Engelhard, G Pasternack, R Cotter, D Hunt, D M Pardoll, and E M Jaffee. The immunodominant major histocompatibility complex class I-restricted antigen of a murine colon tumor derives from an endogenous retroviral gene product. Proc Natl Acad Sci USA.; 93(18): 9730-9735, 1996 Sep. 3.
69. JOHNSON, BARBARA J. B., RICHARD M. KINNEY, CRYSTLE L. KOST AND DENNIS W. TRENT. Molecular Determinants of Alphavirus Neurovirulence: Nucleotide and Deduced Protein Sequence Changes during Attenuation of Venezuelan Equine Encephalitis Virus. J Gen Virol 67:1951-1960, 1986.
70. Aarnoudse, C. A., Krüse, M., Konopitzky, R., Brouwenstijn, N., and Schrier, P. I. (2002). TCR reconstitution in Jurkat reporter cells facilitates the identification of novel tumor antigens by cDNA expression cloning. Int J Cancer 99, 7-13.
71. Alexander, J., Sidney, J., Southwood, S., Ruppert, J., Oseroff, C., Macwal, A., Snoke, K., Serra, H. M., Kubo, R. T., and Sette, A. (1994). Development of high potency universal DR-restricted helper epitopes by modification of high affinity DR-blocking peptides. Immunity 1, 751-761.
72. Banu, N., Chia, A., Ho, Z. Z., Garcia, A. T., Paravasivam, K., Grotenbreg, G. M., Bertoletti, A., and Gehring, A. J. (2014). Building and optimizing a virus-specific T cell receptor library for targeted immunotherapy in viral infections. Scientific Reports 4, 4166.
73. Cornet, S., Miconnet, I., Menez, J., Lemonnier, F., and Kosmatopoulos, K. (2006). Optimal organization of a polypeptide-based candidate cancer vaccine composed of cryptic tumor peptides with enhanced immunogenicity. Vaccine 24, 2102-2109.
74. Depla, E., van der Aa, A., Livingston, B. D., Crimi, C., Allosery, K., de Brabandere, V., Krakover, J., Murthy, S., Huang, M., Power, S., et al. (2008). Rational design of a multiepitope vaccine encoding T-lymphocyte epitopes for treatment of chronic hepatitis B virus infections. Journal of Virology 82, 435-450.
75. Ishioka, G. Y., Fikes, J., Hermanson, G., Livingston, B., Crimi, C., Qin, M., del Guercio, M. F., Oscroff, C., Dahlberg, C., Alexander, J., et al. (1999). Utilization of MHC class I transgenic mice for development of minigene DNA vaccines encoding multiple HLA-restricted CTL epitopes. J Immunol 162, 3915-3925.
76. Janetzki, S., Price, L., Schroeder, H., Britten, C. M., Welters, M. J. P., and Hoos, A. (2015). Guidelines for the automated evaluation of Elispot assays. Nat Protoc 10, 1098-1115.
77. Lyons, G. E., Moore, T., Brasic, N., Li, M., Roszkowski, J. J., and Nishimura, M. I. (2006). Influence of human CD8 on antigen recognition by T-cell receptor-transduced cells. Cancer Res 66, 11455-11461.
78. Nagai, K., Ochi, T., Fujiwara, H., An, J., Shirakata, T., Mineno, J., Kuzushima, K., Shiku, H., Melenhorst, J. J., Gostick, E., et al. (2012). Aurora kinase A-specific T-cell receptor gene transfer redirects T lymphocytes to display effective antileukemia reactivity. Blood 119, 368-376.
79. Panina-Bordignon, P., Tan, A., Termijtelen, A., Demotz, S., Corradin, G., and Lanzavecchia, A. (1989). Universally immunogenic T cell epitopes: promiscuous binding to human MHC class II and promiscuous recognition by T cells. Eur J Immunol 19, 2237-2242.
80. Vitiello, A., Marchesini, D., Furze, J., Sherman, L. A., and Chesnut, R. W. (1991). Analysis of the HLA-restricted influenza-specific cytotoxic T lymphocyte response in transgenic mice carrying a chimeric human-mouse class I major histocompatibility complex. J Exp Med 173, 1007-1015.
81. Yachi, P. P., Ampudia, J., Zal, T., and Gascoigne, N. R. J. (2006). Altered peptide ligands induce delayed CD8-T cell receptor interaction—a role for CD8 in distinguishing antigen quality. Immunity 25, 203-211.
82. Pushko P, Parker M, Ludwig G V, Davis N L, Johnston R E, Smith J F. Replicon-helper systems from attenuated Venezuelan equine encephalitis virus: expression of heterologous genes in vitro and immunization against heterologous pathogens in vivo. Virology. 1997 Dec. 22; 239(2):389-401.
83. Strauss, J H and E G Strauss. The alphaviruses: gene expression, replication, and evolution. Microbiol Rev. 1994 September; 58(3): 491-562.
84. Rhême C, Ehrengruber M U, Grandgirard D. Alphaviral cytotoxicity and its implication in vector development. Exp Physiol. 2005 January; 90(1):45-52. Epub 2004 Nov. 12.
85. Riley, Michael K. I I, and Wilfred Vermerris. Recent Advances in Nanomaterials for Gene Delivery-A Review. Nanomaterials 2017, 7(5), 94.
86. Frolov I, Hardy R, Rice C M. Cis-acting RNA elements at the 5′ end of Sindbis virus genome RNA regulate minus- and plus-strand RNA synthesis. RNA. 2001 November; 7(11): 1638-51.
87. Jose J, Snyder J E, Kuhn R J. A structural and functional perspective of alphavirus replication and assembly. Future Microbiol. 2009 September; 4(7):837-56.
88. Bo Li and C. olin N. Dewey. RSEM: accurate transcript quantification from RNA-Seq data with or without a reference genome. BMC Bioinformatics, 12:323, August 2011
89. Hillary Pearson, Tariq Daouda, Diana Paola Granados, Chantal Durette, Eric Bonneil, Mathieu Courcelles, Anja Rodenbrock, Jean-Philippe Laverdure, Caroline Côté, Sylvie Mader, Sebastien Lemieux, Pierre Thibault, and Claude Perreault. MHC class I-associated peptides derive from selective regions of the human genome. The Journal of Clinical Investigation, 2016,
90. Juliane Liepe, Fabio Marino, John Sidney, Anita Jeko, Daniel E. Bunting, Alessandro Sette, Peter M. Kloetzel, Michael P. H. Stumpf, Albert J. R. Heck, Michele Mishto. A large fraction of HLA class I ligands are proteasome-generated spliced peptides. Science, 21, October 2016.
91. Mommen G P., Marino, F., Meiring H D., Poelen, M C., van Gaans-van den Brink, J A., Mohammed S., Heck A J., and van Els C A. Sampling From the Proteome to the Human Leukocyte Antigen-DR (HLA-DR) Ligandome Proceeds Via High Specificity. Mol Cell Proteomics 15(4): 1412-1423, April 2016.
92. Sebastian Kreiter, Mathias Vormehr, Niels van de Roemer, Mustafa Diken, Martin Lower, Jan Dickmann, Sebastian Boegel, Barbara Schrörs, Fulvia Vascotto, John C. Castle, Arbel D. Tadmor, Stephen P. Schoenberger, Christoph Huber, Özlem Türeci, and Ugur Sahin. Mutant MHC class II epitopes drive therapeutic immune responses to caner. Nature 520, 692-696, April 2015.
93. Tran E., Turcotte S., Gros A., Robbins P. F., Lu Y. C., Dudley M. E., Wunderlich J. R., Somerville R. P., Hogan K., Hinrichs C. S., Parkhurst M. R., Yang J. C., Rosenberg S. A. Cancer immunotherapy based on mutation-specific CD4+ T cells in a patient with epithelial cancer. Science 344(6184) 641-645, May 2014.
94. Andreatta M., Karosiene E., Rasmussen M., Stryhn A., Buus S., Nielsen M. Accurate pan-specific prediction of peptide-MHC class II binding affinity with improved binding core identification. Immunogenetics 67(11-12) 641-650, November 2015.
95. Nielsen, M., Lund, O. NN-align. An artificial neural network-based alignment algorithm for MHC class II peptide binding prediction. BMC Bioinformatics 10:296, September 2009.
96. Nielsen, M., Lundegaard, C., Lund, O. Prediction of MHC class II binding affinity using SMM-align, a novel stabilization matrix alignment method. BMC Bioinformatics 8:238, July 2007.
97. Zhang, J., et al. PEAKS D B: de novo sequencing assisted database search for sensitive and accurate peptide identification. Molecular & Cellular Proteomics. 11(4):1-8. Jan. 2, 2012.
98. Jensen, Kamilla Kjaergaard, et al. “Improved Methods for Predicting Peptide Binding Affinity to MHC Class II Molecules.” Immunology, 2018, doi: 10.1111/imm.12889.
99. Carter, S. L., Cibulskis, K., Helman, E., McKenna, A., Shen, H., Zack, T., Laird, P. W., Onofrio, R. C., Winckler, W., Weir, B. A., et al. (2012). Absolute quantification of somatic DNA alterations in human cancer. Nat. Biotechnol. 30, 413-421
100. McGranahan, N., Rosenthal, R., Hiley, C. T., Rowan, A. J., Watkins, T. B. K., Wilson, G. A., Birkbak, N. J., Vecriah, S., Van Loo, P., Herrero, J., et al. (2017). Allele-Specific HLA Loss and Immune Escape in Lung Cancer Evolution. Cell 171, 1259-1271.e11.
101. Shukla, S. A., Rooney, M. S., Rajasagi, M., Tiao, G., Dixon, P. M., Lawrence, M. S., Stevens, J., Lane, W. J., Dellagatta, J. L., Steelman, S., et al. (2015). Comprehensive analysis of cancer-associated somatic mutations in class I HLA genes. Nat. Biotechnol. 33, 1152-1158.
102. Van Loo, P., Nordgard, S. H., Lingjærde, O. C., Russnes, H. G., Rye, I. H., Sun, W., Weigman, V. J., Marynen, P., Zetterberg, A., Naume, B., et al. (2010). Allele-specific copy number analysis of tumors. Proc. Natl. Acad. Sci. U.S.A 107, 16910-16915.
103. Van Loo, P., Nordgard, S. H., Lingjærde, O. C., Russnes, H. G., Rye, I. H., Sun, W., Weigman, V. J., Marynen, P., Zetterberg, A., Naume, B., et al. (2010). Allele-specific copy number analysis of tumors. Proc. Natl. Acad. Sci. U.S.A 107, 16910-16915.

What is claimed is:

1. A composition for delivery of an antigen expression system, comprising:
the antigen expression system, wherein the antigen expression system comprises at least two distinct coronavirus receptor binding domain (RBD) derived nucleic acid sequences encoding at least two distinct RBD domains, respectively, and wherein the at least two distinct RBD domains are collectively at least 70% identical by amino acid composition to RBD domains from at least two of a clade 1 Sarbecovirus, a clade 2 Sarbecovirus, or a clade 3 Sarbecovirus.

2. A composition for delivery of an antigen expression system, comprising:
the antigen expression system, wherein the antigen expression system comprises:
(a) a vector comprising:
a vector backbone, wherein the vector backbone comprises:
(i) at least one promoter nucleotide sequence, and
(ii) at least one polyadenylation (poly(A)) sequence; and
(b) an antigen cassette, wherein the antigen cassette is inserted into the vector backbone such that the antigen cassette is operably linked to the at least one promoter nucleotide sequence, and wherein the antigen cassette comprises at least two distinct coronavirus receptor binding domain (RBD) derived nucleic acid sequences encoding at least two distinct RBD domains, respectively, and wherein the at least two distinct RBD domains are collectively at least 70% identical by amino acid composition to RBD domains from at least two of a clade 1 Sarbecovirus, a clade 2 Sarbecovirus, or a clade 3 Sarbecovirus.

3. Any one of the above compositions, wherein the at least two distinct RBD domains are collectively at least 70% identical by amino acid composition to RBD domains from each of: (A) the clade 3 Sarbecovirus; and (B) the clade 1 Sarbecovirus and/or the clade 2 Sarbecovirus.

4. Any one of the above compositions, wherein the at least two distinct RBD domains are collectively at least 80% identical by amino acid composition to RBD domains from each of the clade 1 Sarbecovirus and the clade 2 Sarbecovirus.

5. Any one of the above compositions, wherein the at least two distinct RBD domains are collectively at least 85% identical by amino acid composition to RBD domains from each of the clade 1 Sarbecovirus and the clade 2 Sarbecovirus.

6. Any one of the above compositions, wherein the at least two distinct RBD domains are collectively at least 90% identical by amino acid composition to RBD domains from each of the clade 1 Sarbecovirus and the clade 2 Sarbecovirus.

7. Any one of the above compositions, wherein the at least two distinct RBD domains are collectively at least 70% identical by amino acid composition to RBD domains from each of NC_045512.2, NC_004718.3, MT121216.1, DQ648857.1, GQ153542.1, DQ648856.1, AY278489.2, GQ153540.1, MN996532.2, KJ473811.1, NC_014470.1, KC881005.1, MK211377.1, KJ473816.1, MK211376.1, AY572034.1, KP886809.1, MT072864.1, KF569996.1, JX993987.1, MK211378.1, MK211374.1, KJ473815.1, JX993988.1, DQ071615.1, KT444582.1, MZ206298.1, and KJ473814.1.

8. Any one of the above compositions, wherein the antigen expression system comprises at least three distinct coronavirus RBD derived nucleic acid sequences encoding at least three distinct RBD domain.

9. The composition of claim 8, wherein the at least three distinct RBD domains are collectively at least 70% identical by amino acid composition to RBD domains from each of the clade 1 Sarbecovirus, the clade 2 Sarbecovirus, and the clade 3 Sarbecovirus.

10. The composition of claim 8, wherein the at least three distinct RBD domains are collectively at least 70% identical by amino acid composition to RBD domains from each of NC_045512.2, NC_004718.3, MT121216.1, DQ648857.1, GQ153542.1, DQ648856.1, AY278489.2, GQ153540.1, MN996532.2, KJ473811.1, NC_014470.1, KC881005.1, MK211377.1, KJ473816.1, MK211376.1, AY572034.1, KP886809.1, MT072864.1, KF569996.1, JX993987.1, MK211378.1, MK211374.1, KJ473815.1, JX993988.1, DQ071615.1, KT444582.1, MZ206298.1, KJ473814.1, and SARS-COV-2.

11. Any one of the above compositions, wherein the antigen expression system comprises at least four distinct coronavirus RBD derived nucleic acid sequences encoding at least four distinct RBD domain.

12. The composition of claim 11, wherein the at least four distinct RBD domains are collectively at least 70% identical by amino acid composition to RBD domains from each of the clade 1 Sarbecovirus, the clade 2 Sarbecovirus, and the clade 3 Sarbecovirus.

13. The composition of claim 11, wherein the at least four distinct RBD domains are collectively at least 70% identical by amino acid composition to RBD domains from each of NC_045512.2, NC_004718.3, MT121216.1, DQ648857.1, GQ153542.1, DQ648856.1, AY278489.2, GQ153540.1, MN996532.2, KJ473811.1, NC_014470.1, KC881005.1, MK211377.1, KJ473816.1, MK211376.1, AY572034.1, KP886809.1, MT072864.1, KF569996.1, JX993987.1, MK211378.1, MK211374.1, KJ473815.1, JX993988.1, DQ071615.1, KT444582.1, MZ206298.1, KJ473814.1, and SARS-COV2.

14. Any one of the above compositions, wherein each of the sarbecovirus RBD derived nucleic acid sequences are independently derived from RBD nucleic acid sequences of sarbecovirus sequences selected from the group consisting of: KP886809, KJ473815, MK211376, DQ648856, GQ153542, NC_004718, JX993988, and SARS-COV2.

15. The composition of claim 14, wherein each of the sarbecovirus RBD derived nucleic acid sequences are independently derived from RBD nucleic acid sequences of sarbecovirus sequences from each of KP886809, KJ473815, MK211376, and SARS-COV2.

16. The composition of claim 14, wherein each of the sarbecovirus RBD derived nucleic acid sequences are independently derived from RBD nucleic acid sequences of sarbecovirus sequences from each of KJ473815, MK211376, DQ648856, and SARS-COV2.

17. The composition of claim 14, wherein each of the sarbecovirus RBD derived nucleic acid sequences are independently derived from RBD nucleic acid sequences of sarbecovirus sequences from each of GQ153542, NC_004718, JX993988, and SARS-CoV2.

18. Any one of the above compositions, wherein the at least two distinct RBD domains encode full-length RBD domains that are collectively at least 70% identical by amino acid composition to full-length RBD domains from at least two of the clade 1 Sarbecovirus, the clade 2 Sarbecovirus, or the clade 3 Sarbecovirus.

19. Any one of the above compositions, wherein the at least two distinct RBD domains encode full-length RBD domains that are collectively at least 70% identical by amino acid composition to full-length RBD domains from at least two of the clade 1 Sarbecovirus, the clade 2 Sarbecovirus, or the clade 3 Sarbecovirus.

20. Any one of the above compositions, wherein the at least two distinct coronavirus RBD derived nucleic acid sequences comprise at least a betacoronavirus RBD derived nucleic acid sequence.

21. Any one of the above compositions, wherein the at least two distinct coronavirus RBD derived nucleic acid sequences each comprise a betacoronavirus RBD derived nucleic acid sequences.

22. Any one of the above compositions, wherein the at least two distinct coronavirus RBD derived nucleic acid sequences are selected from the group consisting of: a betacoronavirus RBD derived nucleic acid sequence, an alphacoronavirus RBD derived nucleic acid sequence, and combinations thereof.

23. Any one of the above compositions, wherein each of the distinct RBD domains comprises a distinct receptor-binding motif (RBM) domain, respectively, and wherein the distinct RBM domains are collectively at least 30% identical by amino acid composition to RBM domains from at least two of a clade 1 Sarbecovirus, a clade 2 Sarbecovirus, or a clade 3 Sarbecovirus.

24. Any one of the above compositions, wherein the amino acid sequences of the at least two distinct RBD domains other than the amino acid sequences of their respective RBM domains are collectively at least 70% identical by amino acid composition to the amino acid sequences of RBD domains sequences other than the amino acid sequences of an RBM from at least two of a clade 1 Sarbecovirus, a clade 2 Sarbecovirus, or a clade 3 Sarbecovirus.

25. A composition for delivery of an antigen expression system, comprising:
the antigen expression system, wherein the antigen expression system comprises at least two distinct coronavirus receptor binding domain (RBD) derived nucleic acid sequences encoding at least two distinct RBD domains, respectively, and wherein the at least two distinct RBD domains are at least 70% identical by amino acid composition to RBD domains from at least two of a sarbecovirus RBD derived nucleic acid sequence, a merbecoviorus RBD derived nucleic acid sequence, a embecovirus RBD derived nucleic acid sequence, and combinations thereof.

26. Any one of the above compositions, wherein the at least two distinct RBD domains comprises between 2-4, between 2-5, between 2-6, between 2-7, between 2-8, between 2-9, between 2-10, between 2-11, between 2-12, between 2-13, between 2-14, between 2-15, between 2-16, between 2-17, between 2-18, between 2-19, or between 2-20 distinct RBD domains.

27. Any one of the above compositions, wherein the at least two distinct RBD domains comprises between 3-8, between 4-8, between 3-8, between 4-8, between 3-9, between 4-9, between 3-10, or between 4-10 distinct RBD domains.

28. Any one of the above compositions, wherein the at least two distinct coronavirus RBD derived nucleic acid sequences are encoded by a single polynucleotide sequence.

29. The composition of claim 28, wherein the at least two distinct coronavirus RBD derived nucleic acid sequences are encoded by a single antigen cassette.

30. Any one of the above compositions, wherein the at least two distinct coronavirus RBD derived nucleic acid sequences are each encoded by separate polynucleotide sequences.

31. The composition of claim 30, wherein the at least two distinct coronavirus RBD derived nucleic acid sequences are each encoded by separate antigen cassettes.

32. The composition of claim 31, wherein the separate antigen cassettes are each encoded by separate vectors.

33. Any one of the above compositions, wherein each of the at least two distinct coronavirus RBD derived nucleic acid sequences further comprises a distinct trimerization domain derived nucleic acid sequence.

34. Any one of the above compositions, wherein each of the at least two distinct coronavirus RBD derived nucleic acid sequences comprises the same RBD trimerization domain derived nucleic acid sequence.

35. The composition of claim 33 or 34, wherein the RBD trimerization domain is selected from the group consisting of: a T4 trimerization domain, a MTQ trimerization domain; a GCN4 trimerization domain, and combinations thereof.

36. The composition of claim 34, wherein each of the at least two distinct coronavirus RBD derived nucleic acid sequences comprises a T4 trimerization domain derived nucleic acid sequence.

37. The composition of claim 33, wherein the at least two distinct coronavirus RBD derived nucleic acid sequences comprises each independently comprise a T4 trimerization domain, a MTQ trimerization domain; a GCN4 trimerization domain.

38. Any one of the above compositions, wherein the coronavirus RBD derived nucleic acid sequence encodes a full-length RBD domain.

39. Any one of the above compositions, wherein the coronavirus RBD derived nucleic acid sequence encodes a RBD domain (a) lacking a receptor-binding motif (RBM) domain, (b) comprising an RBM domain that is not derived from the coronavirus the remainder of the RBD is derived from, or (c) comprising RBD sequences that are not derived from the coronavirus the RBM domain is derived from.

40. Any one of the above compositions, wherein the coronavirus RBD derived nucleic acid sequence encodes only an RBM domain of the corresponding RBD domain.

41. Any one of the above compositions, wherein the at least two distinct coronavirus RBD derived nucleic acid sequences are linked directly to one another.

42. The composition of claim 41, wherein the at least two distinct coronavirus RBD derived nucleic acid sequences are concatenated such that the sequences are capable of being expressed as a single mRNA.

43. The composition of claim 41 or 42, wherein the at least two distinct coronavirus RBD derived nucleic acid sequences are linked together with a peptide-linker encoding nucleic acid sequence.

44. The composition of claim 43, wherein the peptide-linker encoding nucleic acid sequence encodes a 2A ribosome skipping sequence element, optionally selected from the group consisting of: a E2A ribosome skipping sequence element, a P2A ribosome skipping sequence element, a F2A ribosome skipping sequence element, a T2A sequence ribosome skipping sequence element, and combinations thereof.

45. The composition of claim 43 or 44, wherein the peptide-linker encoding nucleic acid sequence encodes a cleavable peptide linker, optionally selected from: a TEV cleavage site, a furin cleavage site, and combinations thereof.

46. The composition of claim 43, wherein the peptide-linker encoding nucleic acid sequence encodes a T2A sequence ribosome skipping sequence element and a furin cleavage site.

47. Any one of the above compositions, wherein each of the at least two distinct coronavirus RBD derived nucleic acid sequences further comprises a signal-peptide encoding nucleic acid sequence.

48. The composition of claim 47, wherein the signal-peptide comprises a coronavirus derived signal-peptide.

49. The composition of claim 48, wherein the signal-peptide comprises a SARS-COV-2 derived signal-peptide.

50. Any one of the above compositions, wherein one or more of the at least two distinct coronavirus RBD derived nucleic acid sequences are sequence optimized.

51. Any one of the above compositions, wherein the at least two distinct coronavirus RBD derived nucleic acid sequences are operably linked to a promoter.

52. The composition of claim 47, wherein the at least two distinct coronavirus RBD derived nucleic acid sequences are operably linked to a single promoter.

53. The composition of claim 47, wherein each of the at least two distinct coronavirus RBD derived nucleic acid sequences are operably linked to a separate promoter.

54. The composition of any one of claims 47-53, wherein the promoter comprises a subgenomic promoter sequence, optionally wherein the subgenomic promoter sequence comprises an alphavirus derived subgenomic promoter sequence.

55. Any one of the above compositions, wherein each of the at least two distinct coronavirus RBD derived nucleic acid sequences each separately encodes a peptide of the following format: signal peptide-RBD-trimerization domain.

56. Any one of the above compositions, wherein the at least two distinct coronavirus RBD derived nucleic acid sequences encodes a concatenated peptide of the following format: signal peptide-first RBD-first trimerization domain-signal peptide-second RBD-second trimerization domain-signal peptide-third RBD-third trimerization domain.

57. Any one of the above compositions, wherein the at least two distinct coronavirus RBD derived nucleic acid sequences encodes a concatenated peptide of the following format: signal peptide-first RBD-first trimerization domain-T2A-Furin-signal peptide-second RBD-second trimerization domain-T2A-Furin-signal peptide-third RBD-third trimerization domain.

58. Any one of the above compositions, wherein at least one of the at least two distinct RBD domains comprises a SARS-COV-2 SPIKE protein.

59. The composition of claim 58, wherein the SARS-COV-2 SPIKE protein is encoded by a SARS-COV-2 SPIKE derived nucleic acid sequence comprising:
a) a SARS-COV-2 Spike protein comprising a Spike polypeptide sequence as set forth in SEQ ID NO:59 or an epitope-containing fragment thereof, optionally wherein the Spike polypeptide comprises a D614G mutation with reference to SEQ ID NO:59, and optionally wherein the Spike polypeptide is encoded by the nucleotide sequence shown in SEQ ID NO:79, SEQ ID NO:83, SEQ ID NO:85, or SEQ ID NO:87; or
b) a SARS-COV-2 modified Spike protein comprising a mutation selected from the group consisting of: a Spike R682 mutation, a Spike R815 mutation, a Spike K986P mutation, a Spike V987P mutation, and combinations thereof with reference to the Spike polypeptide sequence as set forth in SEQ ID NO:59, and optionally wherein the modified Spike protein comprises a polypeptide sequence as set forth in SEQ ID NO:60 or SEQ ID NO:90 or an epitope-containing fragment thereof.

60. Any one of the above compositions, wherein the antigen expression system comprises at least one coronavirus derived nucleic acid sequence encoding an immunogenic polypeptide distinct from the at least two distinct RBD domains.

61. The composition of claim 58, wherein the at least one coronavirus derived nucleic acid sequence comprises a beta coronavirus derived nucleic acid sequence.

62. Any one of the above compositions, wherein the antigen expression system comprises at least one SARS-COV-2 derived nucleic acid sequence encoding an immunogenic polypeptide.

63. Any one of the above compositions, wherein the antigen cassette comprises at least one SARS-COV-2 derived nucleic acid sequence encoding an immunogenic polypeptide.

64. The composition of claim 62 or 63, wherein the at least one SARS-COV-2 derived nucleic acid sequence comprises a SARS-COV-2 SPIKE derived nucleic acid sequence.

65. The composition of claim 64, wherein the SARS-COV-2 SPIKE derived nucleic acid sequence comprises:
a) a SARS-COV-2 Spike protein comprising a Spike polypeptide sequence as set forth in SEQ ID NO:59 or an epitope-containing fragment thereof, optionally wherein the Spike polypeptide comprises a D614G mutation with reference to SEQ ID NO:59, and optionally wherein the Spike polypeptide is encoded by the nucleotide sequence shown in SEQ ID NO:79, SEQ ID NO:83, SEQ ID NO:85, or SEQ ID NO:87; or
b) a SARS-COV-2 modified Spike protein comprising a mutation selected from the group consisting of: a Spike R682 mutation, a Spike R815 mutation, a Spike K986P mutation, a Spike V987P mutation, and combinations thereof with reference to the Spike polypeptide sequence as set forth in SEQ ID NO:59, and optionally wherein the modified Spike protein comprises a polypeptide sequence as set forth in SEQ ID NO:60 or SEQ ID NO:90 or an epitope-containing fragment thereof.

66. The composition of any one of claims 58-65, wherein the at least one coronavirus derived nucleic acid sequence and/or SARS-COV-2 derived nucleic acid sequence comprises an MHC class I epitope encoding sequence.

67. The composition of any one of claims 58-66, wherein the at least one coronavirus derived nucleic acid sequence, SARS-COV-2 derived nucleic acid sequence, and/or MHC class I epitope encoding sequence comprises:
at least one MHC class I epitope comprising a polypeptide sequence as set forth in Table A,
at least one MHC class II epitope comprising a polypeptide sequence as set forth in Table B,
at least one MHC class I epitope comprising a polypeptide sequence as set forth in Table C, optionally wherein the at least one MHC I epitope is present in a concatenated polypeptide sequence as set forth in SEQ ID NO:57 or SEQ ID NO:58,
at least one polypeptide sequence as set forth in Table 7, or an epitope-containing fragment thereof, optionally wherein the at least one polypeptide sequence is present in a concatenated polypeptide sequence as set forth in SEQ ID NO:92,
at least one polypeptide sequence as set forth in Table 9A, Table 9B, or Table 9C, or an epitope-containing fragment thereof, optionally wherein the at least one polypeptide sequence is present in a concatenated polypeptide comprising each of the sequences set forth in Table 9A, Table 9B, or Table 9C, optionally wherein the concatenated polypeptide comprises the order of sequences set forth in Table 9A, Table 9B, or Table 9C,
at least one MHC class I epitope comprising a polypeptide sequence as set forth in Table A and/or Table C or MHC class II epitope comprising a polypeptide sequence as set forth in Table B, wherein the encoded SARS-COV-2 immunogenic polypeptide is conserved between SARS-COV-2 and a Coronavirus species and/or sub-species other than SARS-COV-2, optionally wherein the Coronavirus species and/or sub-species other than SARS-COV-2 is Severe acute respiratory syndrome (SARS) and/or Middle East respiratory syndrome (MERS),
one or more validated epitopes and/or at least 4, 5, 6, or 7 predicted epitopes, wherein at least 85%, 90%, or 95% of a population carries at least one HLA validated to present at least one of the one or more validated epitopes and/or at least one HLA predicted to present each of the at least 4, 5, 6, or 7 predicted epitopes,
a SARS-COV-2 Spike protein comprising a Spike polypeptide sequence as set forth in SEQ ID NO:59 or an epitope-containing fragment thereof, optionally wherein the Spike polypeptide comprises a D614G mutation with reference to SEQ ID NO:59, and optionally wherein the Spike polypeptide is encoded by the nucleotide sequence shown in SEQ ID NO:79, SEQ ID NO:83, SEQ ID NO:85, or SEQ ID NO:87,
a SARS-COV-2 modified Spike protein comprising a mutation selected from the group consisting of: a Spike R682 mutation, a Spike R815 mutation, a Spike K986P mutation, a Spike V987P mutation, and combinations thereof with reference to the Spike polypeptide sequence as set forth in SEQ ID NO:59, and optionally wherein the modified Spike protein comprises a polypeptide sequence as set forth in SEQ ID NO:60 or SEQ ID NO:90 or an epitope-containing fragment thereof,
a SARS-COV-2 Membrane protein comprising a Membrane polypeptide sequence as set forth in SEQ ID NO:61 or an epitope-containing fragment thereof,
a SARS-COV-2 Nucleocapsid protein comprising a Nucleocapsid polypeptide sequence as set forth in SEQ ID NO:62 or an epitope-containing fragment thereof,
a SARS-COV-2 Envelope protein comprising an Envelope polypeptide sequence as set forth in SEQ ID NO:63 or an epitope-containing fragment thereof,
a variant of any of the above comprising a mutation found in 1% or greater of SARS-CoV-2 subtypes, optionally wherein the variant comprises a SARS-COV-2 variant shown in Table 1, and/or optionally wherein the variant comprises a SARS-COV-2 variant Spike protein comprising a Spike D614G mutation with reference to the Spike polypeptide sequence as set forth in SEQ ID NO:59, a SARS-COV-2 variant Spike protein corresponding to a B.1.351 SARS-COV-2 isolate optionally comprising the Spike polypeptide sequence as set forth in SEQ ID NO:112, or a SARS-COV-2 variant Spike protein corresponding to a B.1.1.7 SARS-COV-2 isolate optionally comprising the Spike polypeptide sequence as set forth in SEQ ID NO:110,
at least one polypeptide sequence as set forth in Table 16A, Table 16B, Table 16C, or Table 16D, or an epitope-containing fragment thereof, optionally wherein the at least one polypeptide sequence is present in a concatenated polypeptide comprising each of the sequences set forth in Table 16A, Table 16B, Table 16C, or Table 16D, optionally wherein the concatenated polypeptide comprises the order of sequences set forth in Table 16A, Table 16B, Table 16C, or Table 16D,
or combinations thereof.

68. The composition of claim 67, wherein the MHC class I epitope encoding sequence comprises at least one polypeptide sequence as set forth in Table 16A, Table 16B, Table 16C, or Table 16D, or an epitope-containing fragment thereof, optionally wherein the at least one polypeptide sequence is present in a concatenated polypeptide comprising each of the sequences set forth in Table 16A, Table 16B, Table 16C, or Table 16D, optionally wherein the concatenated polypeptide comprises the order of sequences set forth in Table 16A, Table 16B, Table 16C, or Table 16D.

69. The composition of any one of claims 58-68, wherein the at least one coronavirus derived nucleic acid sequence, SARS-COV-2 derived nucleic acid sequence, and/or MHC class I epitope encoding sequence is selected from the group consisting of: a Spike protein, a Membrane protein, a Nucleocapsid protein, an Envelope protein, a replicase orf1a and orf1b protein, and combinations thereof.

70. The composition of any one of claims 58-69, wherein the at least one coronavirus derived nucleic acid sequence is encoded on a separate vector distinct from the vector or vectors encoding the at least two distinct coronavirus RBD derived nucleic acid sequences, optionally wherein the encoded immunogenic polypeptide distinct from the at least two distinct coronavirus RBD comprises a MHC class I epitope encoding sequence comprising at least one polypeptide sequence as set forth in Table 16A, Table 16B, Table 16C, or Table 16D, or an epitope-containing fragment thereof, optionally wherein the at least one polypeptide sequence is present in a concatenated polypeptide comprising each of the sequences set forth in Table 16A, Table 16B, Table 16C, or Table 16D, optionally wherein the concatenated polypeptide comprises the order of sequences set forth in Table 16A, Table 16B, Table 16C, or Table 16D.

71. Any one of the above compositions, wherein following administration to a subject, the composition is capable of stimulating an immune response against multiple coronaviruses.

72. Any one of the above compositions, wherein following administration to a subject, the composition is capable of stimulating an immune response against at least each of the coronaviruses from which the at least two distinct coronavirus RBD derived nucleic acid sequences were derived.

73. Any one of the above compositions, wherein following administration to a subject, the composition is capable of stimulating an immune response against a coronavirus distinct from the coronaviruses from which the at least two distinct coronavirus RBD derived nucleic acid sequences were derived.

74. Any one of the above compositions, wherein the composition is capable of stimulating neutralizing antibody production.

75. The composition of claim 74, wherein the composition wherein stimulating neutralizing antibody production comprises a neutralizing antibody titer having an NT50 value calculated as a minimum dilution of sera from the immunized subject that neutralizes a coronavirus by 50%.

76. The composition of claim 74 or 75, wherein stimulating neutralizing antibody production comprises a minimum neutralizing antibody titer for complete neutralization of the coronavirus.

77. Any one of the above compositions, wherein the composition is capable of stimulating antibody production, wherein the antibodies produced are capable of antibody-mediated viral clearance, optionally wherein the antibody-mediated viral clearance comprises Fc-mediated viral clearance.

78. Any one of the above compositions, wherein the antigen expression system comprises one or more vectors, the one or more vectors comprising:
(a) a vector backbone, wherein the vector backbone comprises a chimpanzee adenovirus vector, optionally wherein the chimpanzee adenovirus vector is a ChAdV68 vector, or an alphavirus vector, optionally wherein the alphavirus vector is a Venezuelan equine encephalitis virus vector; and
(b) a cassette encoding the at least two distinct RBD domains, optionally wherein the cassette is integrated between a native promoter nucleotide sequence native to the vector backbone and a poly(A) sequence, optionally wherein the poly(A) sequence is native to the vector backbone.

79. Any one of the above compositions, wherein the antigen expression system comprises one or more vectors, the one or more vectors comprising a vector backbone derived from a Venezuelan equine encephalitis virus.

80. A composition for delivery of an antigen expression system, comprising:
the antigen expression system, wherein the antigen expression system comprises:
(a) optionally, one or more vectors, the one or more vectors comprising:
a vector backbone, wherein the backbone comprises:
(i) at least one promoter nucleotide sequence, and
(ii) at least one polyadenylation (poly(A)) sequence; and
(b) an antigen cassette, optionally wherein the antigen cassette is inserted into the vector backbone when present, and wherein the antigen cassette comprises at least one SARS-CoV-2 derived nucleic acid sequence encoding an immunogenic polypeptide, wherein the immunogenic polypeptide comprises at least one polypeptide sequence as set forth in Table 16A, Table 16B, Table 16C, or Table 16D, or an epitope-containing fragment thereof, optionally wherein the at least one polypeptide sequence is present in a concatenated polypeptide comprising each of the sequences set forth in Table 16A, Table 16B, Table 16C, or Table 16D, optionally wherein the concatenated polypeptide comprises the order of sequences set forth in Table 16A, Table 16B, Table 16C, or Table 16D.

81. The composition of claim 80, wherein the MHC class I epitope encoding sequence comprises at least one polypeptide sequence as set forth in Table 16A, Table 16B, Table 16C, or Table 16D, or an epitope-containing fragment thereof, optionally wherein the at least one polypeptide sequence is present in a concatenated polypeptide comprising each of the sequences set forth in Table 16A, Table 16B, Table 16C, or Table 16D, optionally wherein the concatenated polypeptide comprises the order of sequences set forth in Table 16A, Table 16B, Table 16C, or Table 16D.

82. A pharmaceutical composition comprising the composition of any one of the above claims and a pharmaceutically acceptable carrier.

83. An isolated nucleotide sequence or set of isolated nucleotide sequences comprising the antigen expression system and/or the antigen cassette of any one of the above claims.

84. A vector or set of vectors comprising the antigen expression system, the antigen cassette nucleotide sequence, and/or the isolated nucleotide sequence of any one of the above claims.

85. A method for treating a coronavirus infection or preventing a coronavirus infection in a subject, the method comprising administering to the subject the composition of any of the above composition claims.

86. A method for inducing an immune response in a subject, the method comprising administering to the subject the composition of any of the above composition claims.

87. The method of claim 85 or 86, wherein the method comprises a homologous prime/boost strategy.

88. The method of claim 85 or 86, wherein the method comprises a heterologous prime/boost strategy, optionally wherein the heterologous prime/boost strategy comprises (a) an identical antigen cassette encoded by different vaccine platforms, (b) different antigen cassettes encoded by the same vaccine platform, and/or (c) different antigen cassettes encoded by different vaccine platforms.

89. The method of any one of claims 85-88, wherein the method comprises administering a vector or vectors encoding the at least two distinct coronavirus RBD derived nucleic acid sequences and administering a vector or vectors encoding at least one coronavirus derived nucleic acid sequence encoding an immunogenic polypeptide distinct from the at least two distinct RBD domains.

90. The method of claim 89, wherein the vector or vectors encoding the at least two distinct coronavirus RBD derived nucleic acid sequences and the vector or vectors encoding at least one coronavirus derived nucleic acid sequence are co-formulated.

91. The method of claim 89, wherein the vector or vectors encoding the at least two distinct coronavirus RBD derived nucleic acid sequences and the vector or vectors encoding at least one coronavirus derived nucleic acid sequence are administered separately.

92. The method of claim 91, wherein the vector or vectors encoding the at least two distinct coronavirus RBD derived nucleic acid sequences and the vector or vectors encoding at least one coronavirus derived nucleic acid sequence are administered concurrently.

93. The method of any one of claims 89-92, wherein the at least one coronavirus derived nucleic acid sequence comprises:
at least one MHC class I epitope comprising a polypeptide sequence as set forth in Table A,
at least one MHC class II epitope comprising a polypeptide sequence as set forth in Table B,
at least one MHC class I epitope comprising a polypeptide sequence as set forth in Table C, optionally wherein the at least one MHC I epitope is present in a concatenated polypeptide sequence as set forth in SEQ ID NO:57 or SEQ ID NO:58,
at least one polypeptide sequence as set forth in Table 7, or an epitope-containing fragment thereof, optionally wherein the at least one polypeptide sequence is present in a concatenated polypeptide sequence as set forth in SEQ ID NO:92,
at least one polypeptide sequence as set forth in Table 9A, Table 9B, or Table 9C, or an epitope-containing fragment thereof, optionally wherein the at least one polypeptide sequence is present in a concatenated polypeptide comprising each of the sequences set forth in Table 9A, Table 9B, or Table 9C, optionally wherein the concatenated polypeptide comprises the order of sequences set forth in Table 9A, Table 9B, or Table 9C,
at least one MHC class I epitope comprising a polypeptide sequence as set forth in Table A and/or Table C or MHC class II epitope comprising a polypeptide sequence as set forth in Table B, wherein the encoded SARS-COV-2 immunogenic polypeptide is conserved between SARS-COV-2 and a Coronavirus species and/or sub-species other than SARS-COV-2, optionally wherein the Coronavirus species and/or sub-species other than SARS-COV-2 is Severe acute respiratory syndrome (SARS) and/or Middle East respiratory syndrome (MERS),
one or more validated epitopes and/or at least 4, 5, 6, or 7 predicted epitopes, wherein at least 85%, 90%, or 95% of a population carries at least one HLA validated to present at least one of the one or more validated epitopes and/or at least one HLA predicted to present each of the at least 4, 5, 6, or 7 predicted epitopes,
a SARS-COV-2 Spike protein comprising a Spike polypeptide sequence as set forth in SEQ ID NO:59 or an epitope-containing fragment thereof, optionally wherein the Spike polypeptide comprises a D614G mutation with reference to SEQ ID NO:59, and optionally wherein the Spike polypeptide is encoded by the nucleotide sequence shown in SEQ ID NO:79, SEQ ID NO:83, SEQ ID NO:85, or SEQ ID NO:87,
a SARS-COV-2 modified Spike protein comprising a mutation selected from the group consisting of: a Spike R682 mutation, a Spike R815 mutation, a Spike K986P mutation, a Spike V987P mutation, and combinations thereof with reference to the Spike polypeptide sequence as set forth in SEQ ID NO:59, and optionally wherein the modified Spike protein comprises a polypeptide sequence as set forth in SEQ ID NO:60 or SEQ ID NO:90 or an epitope-containing fragment thereof,
a SARS-COV-2 Membrane protein comprising a Membrane polypeptide sequence as set forth in SEQ ID NO:61 or an epitope-containing fragment thereof,
a SARS-COV-2 Nucleocapsid protein comprising a Nucleocapsid polypeptide sequence as set forth in SEQ ID NO:62 or an epitope-containing fragment thereof,
a SARS-COV-2 Envelope protein comprising an Envelope polypeptide sequence as set forth in SEQ ID NO:63 or an epitope-containing fragment thereof,
a variant of any of the above comprising a mutation found in 1% or greater of SARS-CoV-2 subtypes, optionally wherein the variant comprises a SARS-COV-2 variant shown in Table 1, and/or optionally wherein the variant comprises a SARS-COV-2 variant Spike protein comprising a Spike D614G mutation with reference to the Spike polypeptide sequence as set forth in SEQ ID NO:59, a SARS-COV-2 variant Spike protein corresponding to a B.1.351 SARS-COV-2 isolate optionally comprising the Spike polypeptide sequence as set forth in SEQ ID NO:112, or a SARS-COV-2 variant Spike protein corresponding to a B.1.1.7 SARS-COV-2 isolate optionally comprising the Spike polypeptide sequence as set forth in SEQ ID NO:110,
at least one polypeptide sequence as set forth in Table 16A, Table 16B, Table 16C, or Table 16D, or an epitope-containing fragment thereof, optionally wherein the at least one polypeptide sequence is present in a concatenated polypeptide comprising each of the sequences set forth in Table 16A, Table 16B, Table 16C, or Table 16D, optionally wherein the concatenated polypeptide comprises the order of sequences set forth in Table 16A, Table 16B, Table 16C, or Table 16D,
or combinations thereof.

94. The method of any one of claims 89-92, wherein the at least one coronavirus derived nucleic acid sequence comprises a MHC class I epitope encoding sequence comprising at least one polypeptide sequence as set forth in Table 16A, Table 16B, Table 16C, or Table 16D, or an epitope-containing fragment thereof, optionally wherein the at least one polypeptide sequence is present in a concatenated polypeptide comprising each of the sequences set forth in Table 16A, Table 16B, Table 16C, or Table 16D, optionally wherein the concatenated polypeptide comprises the order of sequences set forth in Table 16A, Table 16B, Table 16C, or Table 16D.

95. A method of manufacturing the vector or set of vectors, the antigen expression system. the antigen cassette nucleotide sequence, and/or the isolated nucleotide sequence of any one of the above composition claims.
